(19) United States
(12) Patent Application Publication
(10) Pub. No.: US 2025/0302979 A1
(43) Pub. Date: Oct. 2, 2025

(54) ANTIBODY-OLIGONUCLEOTIDE CONJUGATE AND ANTIBODY-PEPTIDE-OLIGONUCLEOTIDE CONJUGATE COMPOSITIONS AND METHODS OF INDUCING EXON SKIPPING

(71) Applicant: Avidity Biosciences, Inc., San Diego, CA (US)

(72) Inventors: Isaac MARKS, San Diego, CA (US); Tyler ALBIN, La Jolla, CA (US); Michael C. COCHRAN, La Jolla, CA (US); Venkata Ramana DOPPALAPUDI, San Diego, CA (US)

(21) Appl. No.: 18/553,134

(22) PCT Filed: Apr. 1, 2022
(86) PCT No.: PCT/US2022/023123

§ 371 Date: Sep. 28, 2023

Related U.S. Application Data
(60) Provisional application No. 63/170,388, filed on Apr. 2, 2021.

(51) Int. Cl. A61K 47/68; A61P 21/00; C07K 16/28

(52) U.S. Cl. CPC A61K 47/6807; A61K 47/6849; A61K 47/6883; A61P 21/00; C07K 16/2881

(57) ABSTRACT

Disclosed herein are molecules and pharmaceutical compositions that induce an insertion, deletion, duplication, or alteration in an incorrectly spliced mRNA transcript to induce exon skipping or exon inclusion. Also described herein include methods for treating a disease or disorder that comprises a molecule or a pharmaceutical composition that induces an insertion, deletion, duplication, or alteration in an incorrectly spliced mRNA transcript to induce exon skipping or exon inclusion.

### CROSS-REFERENCE

This application is a U.S. National Phase entry of International Application No. PCT/US2022/023123, filed on Apr. 1, 2022, which claims the benefit of U.S. Provisional Application Ser. No. 63/170,388 filed on Apr. 2, 2021, each of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Apr. 25, 2025, is named 45532-752_831_SL.txt and is 233,624 bytes bytes in size.

### BACKGROUND OF THE DISCLOSURE

Modulation of RNA function is a developing area of therapeutic interest. Drugs that affect mRNA stability like antisense oligonucleotides and short interfering RNAs are one way to modulate RNA function. Another group of oligonucleotides can modulate RNA function by altering the processing of pre-mRNA to include or exclude specific regions of pre-mRNAs from the ultimate gene product: the encoded protein. As such, oligonucleotide therapeutics represent a means of modulating protein expression in disease states and as such have utility as therapeutics.

### SUMMARY OF THE DISCLOSURE

Disclosed herein, in certain aspects, are molecules and pharmaceutical compositions for modulating RNA processing.

Disclosed herein, in certain aspects, are methods of treating a disease or disorder that can be modulated by the processing of the pre-mRNA transcript having an incorrectly spliced mRNA transcript in a subject in need thereof, the method comprising: administering to the subject a polynucleic acid molecule conjugate; wherein the polynucleic acid molecule conjugate is conjugated to a cell targeting binding moiety; wherein the polynucleotide optionally comprises at least one 2′ modified nucleotide, at least one modified internucleotide linkage, or at least one inverted abasic moiety; wherein the polynucleic acid molecule conjugate induces insertion, deletion, duplication, or alteration in the incorrectly spliced mRNA transcript to induce exon skipping or exon inclusion in the incorrectly spliced mRNA transcript to generate a fully processed mRNA transcript; and wherein the fully processed mRNA transcript encodes a functional protein, thereby treating the disease or disorder in the subject. In some aspects, the disease or disorder is further characterized by one or more mutations in the mRNA. In some aspects, the disease or disorder comprises a neuromuscular disease, a genetic disease, cancer, a hereditary disease, or a cardiovascular disease. In some aspects, the disease or disorder is muscular dystrophy. In some aspects, the disease or disorder is Duchenne muscular dystrophy. In some aspects, the exon skipping is of exon 8, 23, 35, 43, 44, 45, 50, 51, 52, 53, or 55 of the DMD gene. In some aspects, the exon skipping is of exon 23 of the DMD gene. In some aspects, the polynucleic acid molecule conjugate comprises a structure of Formula (I):

A-X-B Formula (I)
wherein,
A comprises a binding moiety;
B consists of a polynucleotide; and
X consists of a bond or first linker.

In some aspects, the polynucleic acid molecule conjugate comprises a structure of Formula (II):

A-X-B-Y-C Formula (II)
wherein,
A comprises a binding moiety;
B consists of a polynucleotide;
C consists of a polymer;
X consists of a bond or first linker; and
Y consists of a bond or second linker.

In some aspects, the polynucleic acid molecule conjugate comprises a structure of Formula (III):

A-X-C-Y-B Formula (III)
wherein,
A comprises a binding moiety;
B consists of a polynucleotide;
C consists of a polymer;
X consists of a bond or first linker; and
Y consists of a bond or second linker.

In some aspects, the at least one 2′ modified nucleotide comprises a morpholino, 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleotide. In some aspects, the at least one 2′ modified nucleotide comprises locked nucleic acid (LNA), ethylene nucleic acid (ENA), or a peptide nucleic acid (PNA). In some aspects, the at least one 2′ modified nucleotide comprises a morpholino. In some aspects, the at least one inverted basic moiety is at least one terminus. In some aspects, the at least one modified internucleotide linkage comprises a phosphorothioate linkage or a phosphorodithioate linkage. In some aspects, the polynucleic acid molecule is at least from about 10 to about 30 nucleotides in length. In some aspects, the polynucleic acid molecule is at least one of: from about 15 to about 30, from about 18 to about 25, from about 18 to about 24, from about 19 to about 23, or from about 20 to about 22 nucleotides in length. In some aspects, the polynucleic acid molecule is at least about 16, 17, 18, 19, 20, 21, 22, 23, 24, or 25 nucleotides in length. In some aspects, the polynucleic acid molecule comprises at least one of: from about 5% to about 100% modification, from about 10% to about 100% modification, from about 20% to about 100% modification, from about 30% to about 100% modification, from about 40% to about 100% modification, from about 50% to about 100% modification, from about 60% to about 100% modification, from about 70% to about 100% modification, from about 80% to about 100% modification, and from about 90% to about 100% modification. In some aspects, the polynucleic acid molecule comprises at least one of: from about 10% to about 90% modification, from about 20% to about 90% modification, from about 30% to about 90% modification, from about 40% to about 90% modification, from about 50% to about 90% modification, from about 60% to about 90% modification, from about 70% to about 90% modification, and from about 80% to about 100% modification. In some aspects, the polynucleic acid molecule comprises at least one of: from about 10% to about 80% modification, from about 20% to about 80% modification, from about 30% to about 80% modification, from about 40% to about 80% modification, from about 50% to about 80% modification, from about 60% to about 80% modification, and from about 70% to about 80% modification. In some aspects, the polynucleic acid molecule comprises at least one of: from about 10% to about 70% modification, from about 20% to about 70% modification, from about 30% to about 70% modification, from about 40% to about 70% modification, from about 50% to about 70% modification, and from about 60% to about 70% modification. In some aspects, the polynucleic acid molecule comprises at least one of: from about 10% to about 60% modification, from about 20% to about 60% modification, from about 30% to about 60% modification, from about 40% to about 60% modification, and from about 50% to about 60% modification. In some aspects, the polynucleic acid molecule comprises at least one of: from about 10% to about 50% modification, from about 20% to about 50% modification, from about 30% to about 50% modification, and from about 40% to about 50% modification. In some aspects, the polynucleic acid molecule comprises at least one of: from about 10% to about 40% modification, from about 20% to about 40% modification, and from about 30% to about 40% modification. In some aspects, the polynucleic acid molecule comprises at least one of: from about 10% to about 30% modification, and from about 20% to about 30% modification. In some aspects, the polynucleic acid molecule comprises from about 10% to about 20% modification. In some aspects, the polynucleic acid molecule comprises from about 15% to about 90%, from about 20% to about 80%, from about 30% to about 70%, or from about 40% to about 60% modifications. In some aspects, the polynucleic acid molecule comprises at least about 15%, 20%, 30%, 40%, 50%, 60%, 70%, 80%, 90%, 95%, or 99% modification. In some aspects, the polynucleic acid molecule comprises at least about 3, about 4, about 5, about 6, about 7, about 8, about 9, about 10, about 11, about 12, about 13, about 14, about 15, about 16, about 17, about 18, about 19, about 20, about 21, about 22 or more modifications. In some aspects, the polynucleic acid molecule comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22 or more modified nucleotides. In some aspects, the polynucleic acid molecule comprises a single strand. In some aspects, the polynucleic acid molecule comprises two or more strands. In some aspects, the polynucleic acid molecule comprises a first polynucleotide and a second polynucleotide hybridized to the first polynucleotide to form a double-stranded polynucleic acid molecule. In some aspects, the second polynucleotide comprises at least one modification. In some aspects, the first polynucleotide and the second polynucleotide are RNA molecules. In some aspects, the first polynucleotide and the second polynucleotide are siRNA molecules. In some aspects, X and Y are independently a bond, a degradable linker, a non-degradable linker, a cleavable linker, or a non-polymeric linker group. In some aspects, X is a bond. In some aspects, X is a C₁-C₆ alkyl group. In some aspects, Y is a C₁-C₆ alkyl group. In some aspects, X is a homobifunctional linker or a heterobifunctional linker, optionally conjugated to a C₁-C₆ alkyl group. In some aspects, Y is a homobifunctional linker or a heterobifunctional linker. In some aspects, the binding moiety is an antibody or binding fragment thereof. In some aspects, the antibody or binding fragment thereof comprises a humanized antibody or binding fragment thereof, chimeric antibody or binding fragment thereof, monoclonal antibody or binding fragment thereof, monovalent Fab′, divalent Fab2, single-chain variable fragment (scFv), diabody, minibody, nanobody, single-domain antibody (sdAb), or camelid antibody or binding fragment thereof. In some aspects, C is polyethylene glycol. In some aspects, C has a molecular weight of about 5000 Da. In some aspects, A-X is conjugated to the 5′ end of B and Y—C is conjugated to the 3′ end of B. In some aspects, Y—C is conjugated to the 5′ end of B and A-X is conjugated to the 3′ end of B. In some aspects, A-X, Y—C or a combination thereof is conjugated to an intemucleotide linkage group. In some aspects, methods further comprise D. In some aspects, D is conjugated to C or to A. In some aspects, D is conjugated to the molecule conjugate of Formula (II) according to Formula (IV):

(A-X-B-Y-Ce)-L-D Formula (IV)
wherein,
A comprises a binding moiety;
B consists of a polynucleotide;
C consists of a polymer;
X consists of a bond or first linker;
Y consists of a bond or second linker;
L consists of a bond or third linker;
D consists of an endosomolytic moiety; and
c is an integer between 0 and 1; and
wherein the polynucleotide comprises at least one 2′ modified nucleotide, at least one modified intemucleotide linkage, or an inverted abasic moiety; and D is conjugated anywhere on A, B, or C.

In some aspects, D is INF7 or melittin. In some aspects, L is a C₁-C₆ alkyl group. In some aspects, L is a homobifunctional linker or a heterobifunctional linker. In some aspects, methods further comprise at least a second binding moiety A. In some aspects, the at least second binding moiety A is conjugated to A, to B, or to C.

Disclosed herein, in certain aspects, are methods of treating a disease or disorder that can be modulated by the processing of the pre-mRNA transcript having an incorrectly spliced mRNA transcript in a subject in need thereof, the method comprising: administering to the subject an antibody-peptide-oligonucleotide conjugate (APOC) or an antibody-peptide-polynucleic acid molecule conjugate; wherein the antibody is conjugated to a peptide or the oligonucleotide/polynucleic acid; wherein the peptide is conjugated to an oligonucleotide or polynucleic acid molecule; wherein the polynucleotide optionally comprises at least one 2′ modified nucleotide, at least one modified internucleotide linkage, or at least one inverted abasic moiety; wherein the antibody-peptide-oligonucleotide conjugate (APOC) or antibody-peptide-polynucleic acid molecule conjugate induces insertion, deletion, duplication, or alteration in the incorrectly spliced mRNA transcript to induce exon skipping or exon inclusion in the incorrectly spliced mRNA transcript to generate a fully processed mRNA transcript; and wherein the fully processed mRNA transcript encodes a functional protein, thereby treating the disease or disorder in the subject. In some aspects, the disease or disorder is further characterized by one or more mutations in the pre-mRNA. In some aspects, the disease or disorder comprises a neuromuscular disease, a genetic disease, cancer, a hereditary disease, or a cardiovascular disease. In some aspects, the disease or disorder is muscular dystrophy. In some aspects, the disease or disorder is Duchenne muscular dystrophy. In some aspects, the exon skipping is of exon 8, 23, 35, 43, 44, 45, 50, 51, 52, 53, or 55 of the DMD gene. In some aspects, the exon skipping is of exon 23 of the DMD gene.

In some aspects, the antibody-peptide-oligonucleotide conjugate (APOC) or antibody-peptide-polynucleic acid molecule conjugate comprises a structure of Formula (V):

A-(X₁-B-X₂-D)n Formula (V)
wherein,
   A is an antibody or antigen binding fragment thereof;
   B is a polynucleotide;
   D is an endosomolytic peptide or a membrane penetrating peptide;
   X₁ is a bond or first non-polymeric linker; and
   X₂ is an optional bond or optional second linker;
   n is an integer ≥1.

In some aspects, the antibody-peptide-polynucleic acid molecule conjugate or antibody-peptide-oligonucleotide conjugate comprises a structure of Formula (VI):

A-(X₁-D-X₂-B)n Formula (VI)
wherein,
   A is an antibody or antigen binding fragment thereof;
   B is a polynucleotide;
   D is an endosomolytic peptide or a membrane penetrating peptide;
   X₁ is a bond or first non-polymeric linker; and
   X₂ is an optional bond or optional second linker;
   n is an integer ≥1.

In some aspects, the antibody-peptide-polynucleic acid molecule conjugate or antibody-peptide-oligonucleotide conjugate comprises a structure of Formula (VII):

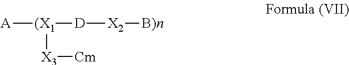

wherein,
   A is an antibody or antigen binding fragment thereof;
   B is a polynucleotide;
   D is an endosomolytic peptide or a membrane penetrating peptide
   C is a polymer;
   X₁ is a bond or first non-polymeric linker;
   X₂ is an optional bond or optional second linker;
   X₃ is an optional bond or optional third linker;
   n is an integer ≥1;
   m is an integer ≥1.

Disclosed herein, in some aspects, are methods of inducing an insertion, deletion, duplication, or alteration in the incorrectly spliced mRNA transcript to induce exon skipping or exon inclusion in the incorrectly spliced mRNA transcript, the method comprising: contacting a target cell with a polynucleic acid molecule conjugate (e.g., antibody-peptide-polynucleic acid molecule conjugate), wherein the polynucleotide comprises at least one 2′ modified nucleotide, at least one modified intemucleotide linkage, or at least one inverted abasic moiety; hybridizing the polynucleic acid molecule conjugate to the incorrectly spliced mRNA transcript within the target cell to induce an insertion, deletion, duplication, or alteration in the incorrectly spliced mRNA transcript to induce exon skipping or exon inclusion, wherein the incorrectly spliced mRNA transcript is capable of encoding a functional form of a protein; and translating the functional form of a protein from a fully processed mRNA transcript of the previous step. In some aspects, the target cell is a target cell of a subject. In some aspects, the incorrectly spliced mRNA transcript further induces a disease or disorder. In some aspects, the disease or disorder is further characterized by one or more mutations in the mRNA. In some aspects, the disease or disorder comprises a neuromuscular disease, a genetic disease, cancer, a hereditary disease, or a cardiovascular disease. In some aspects, the disease or disorder is muscular dystrophy. In some aspects, the disease or disorder is Duchenne muscular dystrophy. In some aspects, the exon skipping is of exon 8, 23, 35, 43, 44, 45, 50, 51, 52, 53, or 55 of the DMD gene. In some aspects, the exon skipping is of exon 23 of the DMD gene. In some aspects, the polynucleic acid molecule conjugate comprises a structure of Formula (I):

A-X-B Formula (I)
wherein,
A comprises a binding moiety;
B consists of a polynucleotide; and
X consists of a bond or first linker.

In some aspects, the polynucleic acid molecule conjugate comprises a structure of Formula (II):

A-X-B-Y-C Formula (II)
wherein,
A comprises a binding moiety;
B consists of a polynucleotide;
C consists of a polymer;
X consists of a bond or first linker; and
Y consists of a bond or second linker.

In some aspects, the polynucleic acid molecule conjugate comprises a structure of Formula (III):

A-X-C-Y-B Formula (III)
wherein,
A comprises a binding moiety;
B consists of a polynucleotide;
C consists of a polymer;
X consists of a bond or first linker; and
Y consists of a bond or second linker.

In some aspects, the at least one 2′ modified nucleotide comprises a morpholino, 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleotide. In some aspects, the at least one 2′ modified nucleotide comprises locked nucleic acid (LNA), ethylene nucleic acid (ENA), peptide nucleic acid (PNA). In some aspects, the at least one 2′ modified nucleotide comprises a morpholino. In some aspects, the at least one inverted basic moiety is at least one terminus. In some aspects, the at least one modified internucleotide linkage comprises a phosphorothioate linkage or a phosphorodithioate linkage. In some aspects, the polynucleic acid molecule is at least from about 10 to about 30 nucleotides in length. In some aspects, the polynucleic acid molecule is at least one of: from about 15 to about 30, from about 18 to about 25, from about 18 to about 24, from about 19 to about 23, or from about 20 to about 22 nucleotides in length. In some aspects, the polynucleic acid molecule is at least about 16, 17, 18, 19, 20, 21, 22, 23, 24, or 25 nucleotides in length. In some aspects, the polynucleic acid molecule comprises at least one of: from about 5% to about 100% modification, from about 10% to about 100% modification, from about 20% to about 100% modification, from about 30% to about 100% modification, from about 40% to about 100% modification, from about 50% to about 100% modification, from about 60% to about 100% modification, from about 70% to about 100% modification, from about 80% to about 100% modification, and from about 90% to about 100% modification. In some aspects, the polynucleic acid molecule comprises at least one of: from about 10% to about 90% modification, from about 20% to about 90% modification, from about 30% to about 90% modification, from about 40% to about 90% modification, from about 50% to about 90% modification, from about 60% to about 90% modification, from about 70% to about 90% modification, and from about 80% to about 100% modification. In some aspects, the polynucleic acid molecule comprises at least one of: from about 10% to about 80% modification, from about 20% to about 80% modification, from about 30% to about 80% modification, from about 40% to about 80% modification, from about 50% to about 80% modification, from about 60% to about 80% modification, and from about 70% to about 80% modification. In some aspects, the polynucleic acid molecule comprises at least one of: from about 10% to about 70% modification, from about 20% to about 70% modification, from about 30% to about 70% modification, from about 40% to about 70% modification, from about 50% to about 70% modification, and from about 60% to about 70% modification. In some aspects, the polynucleic acid molecule comprises at least one of: from about 10% to about 60% modification, from about 20% to about 60% modification, from about 30% to about 60% modification, from about 40% to about 60% modification, and from about 50% to about 60% modification. In some aspects, the polynucleic acid molecule comprises at least one of: from about 10% to about 50% modification, from about 20% to about 50% modification, from about 30% to about 50% modification, and from about 40% to about 50% modification. In some aspects, the polynucleic acid molecule comprises at least one of: from about 10% to about 40% modification, from about 20% to about 40% modification, and from about 30% to about 40% modification. In some aspects, the polynucleic acid molecule comprises at least one of: from about 10% to about 30% modification, and from about 20% to about 30% modification. In some aspects, the polynucleic acid molecule comprises from about 10% to about 20% modification. In some aspects, the polynucleic acid molecule comprises from about 15% to about 90%, from about 20% to about 80%, from about 30% to about 70%, or from about 40% to about 60% modifications. In some aspects, the polynucleic acid molecule comprises at least about 15%, 20%, 30%, 40%, 50%, 60%, 70%, 80%, 90%, 95%, or 99% modification. In some aspects, the polynucleic acid molecule comprises at least about 3, about 4, about 5, about 6, about 7, about 8, about 9, about 10, about 11, about 12, about 13, about 14, about 15, about 16, about 17, about 18, about 19, about 20, about 21, about 22 or more modifications. In some aspects, the polynucleic acid molecule comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22 or more modified nucleotides. In some aspects, the polynucleic acid molecule comprises a single strand. In some aspects, the polynucleic acid molecule comprises two or more strands. In some aspects, the polynucleic acid molecule comprises a first polynucleotide and a second polynucleotide hybridized to the first polynucleotide to form a double-stranded polynucleic acid molecule. In some aspects, the second polynucleotide comprises at least one modification. In some aspects, the first polynucleotide and the second polynucleotide are RNA molecules. In some aspects, the first polynucleotide and the second polynucleotide are siRNA molecules. In some aspects, X and Y are independently a bond, a degradable linker, a non-degradable linker, a cleavable linker, or a non-polymeric linker group. In some aspects, X is a bond. In some aspects, X is a C₁-C₆ alkyl group. In some aspects, Y is a C₁-C₆ alkyl group. In some aspects, X is a homobifunctional linker or a heterobifunctional linker, optionally conjugated to a C₁-C₆ alkyl group. In some aspects, Y is a homobifunctional linker or a heterobifunctional linker. In some aspects, the binding moiety is an antibody or antigen binding fragment thereof. In some aspects, the antibody or antigen binding fragment thereof comprises a humanized antibody or antigen binding fragment thereof, chimeric antibody or antigen binding fragment thereof, monoclonal antibody or binding fragment thereof, monovalent Fab′, divalent Fab2, single-chain variable fragment (scFv), diabody, minibody, nanobody, single-domain antibody (sdAb), or camelid antibody or binding fragment thereof. In some aspects, C is polyethylene glycol. In some aspects, C has a molecular weight of about 5000 Da. In some aspects, A-X is conjugated to the 5′ end of B and Y—C is conjugated to the 3′ end of B. In some aspects, Y—C is conjugated to the 5′ end of B and A-X is conjugated to the 3′ end of B. In some aspects, A-X, Y—C or a combination thereof is conjugated to an intemucleotide linkage group. In some aspects, methods further comprise D. In some aspects, D is conjugated to C or to A. In some aspects, D is conjugated to the molecule conjugate of Formula (II) according to Formula (IV):

(A-X-B-Y-Ce)-L-D Formula (IV)
wherein,
A comprises a binding moiety;
B consists of a polynucleotide;
C consists of a polymer;
X consists of a bond or first linker;
Y is a bond or second linker;
L consists of a bond or third linker;
D consists of an endosomolytic moiety; and
c is an integer between 0 and 1; and
wherein the polynucleotide comprises at least one 2′ modified nucleotide, at least one modified intemucleotide linkage, or an inverted abasic moiety; and D is conjugated anywhere on A, B, or C.

In some aspects, D is INF7 or melittin. In some aspects, L is a C₁-C₆ alkyl group. In some aspects, L is a homobifunctional linker or a heterobifunctional linker. In some aspects, methods further comprise at least a second binding moiety A. In some aspects, the at least second binding moiety A is conjugated to A, to B, or to C. In some aspects, the method is an in vivo method. In some aspects, the method is an in vitro method. In some aspects, the subject is a human.

Disclosed herein, in some aspects, are methods of inducing an insertion, deletion, duplication, or alteration in the incorrectly spliced mRNA transcript to induce exon skipping or exon inclusion in the incorrectly spliced mRNA transcript, the method comprising: contacting a target cell with an antibody-peptide-oligonucleotide conjugate (APOC) or antibody-peptide-polynucleic acid molecule conjugate, wherein the oligonucleotide comprises at least one 2′ modified nucleotide, at least one modified intemucleotide linkage, or at least one inverted abasic moiety; hybridizing the polynucleic acid molecule conjugate to the incorrectly spliced mRNA transcript within the target cell to induce an insertion, deletion, duplication, or alteration in the incorrectly spliced mRNA transcript to induce exon skipping or exon inclusion, wherein the incorrectly spliced mRNA transcript is capable of encoding a functional form of a protein; and translating the functional form of a protein from a fully processed mRNA transcript of the previous step. In some aspects, the target cell is a target cell of a subject. In some aspects, the incorrectly spliced mRNA transcript further induces a disease or disorder. In some aspects, the disease or disorder is further characterized by one or more mutations in the mRNA. In some aspects, the disease or disorder comprises a neuromuscular disease, a genetic disease, cancer, a hereditary disease, or a cardiovascular disease. In some aspects, the disease or disorder is muscular dystrophy. In some aspects, the disease or disorder is Duchenne muscular dystrophy. In some aspects, the exon skipping is of exon 8, 23, 35, 43, 44, 45, 50, 51, 52, 53, or 55 of the DMD gene. In some aspects, the exon skipping is of exon 23 of the DMD gene.

In some aspects, the antibody-peptide-oligonucleotide conjugate (APOC) or antibody-peptide-polynucleic acid molecule conjugate comprises a structure of Formula (V):

A-(X₁-B-X₂-D)n Formula (V)
wherein,
   A is an antibody or antigen binding fragment thereof;
   B is a polynucleotide;
   D is an endosomolytic peptide or a membrane penetrating peptide;
   X₁ is a bond or first non-polymeric linker; and
   X₂ is an optional bond or optional second linker;
   n is an integer ≥1.

In some aspects, the antibody-peptide-polynucleic acid molecule conjugate or antibody-peptide-oligonucleotide conjugate comprises a structure of Formula (VI):

A-(X₁-D-X₂-B)n Formula (VI)
wherein,
   A is an antibody or antigen binding fragment thereof;
   B is a polynucleotide;
   D is an endosomolytic peptide or a membrane penetrating peptide;
   X₁ is a bond or first non-polymeric linker; and
   X₂ is an optional bond or optional second linker;
   n is an integer ≥1.

In some aspects, the antibody-peptide-polynucleic acid molecule conjugate or antibody-peptide-oligonucleotide conjugate comprises a structure of Formula (VII):

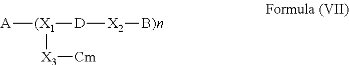

wherein,
   A is an antibody or antigen binding fragment thereof;
   B is a polynucleotide;
   D is an endosomolytic peptide or a membrane penetrating peptide;
   C is a polymer;
   X₁ is a bond or first non-polymeric linker;
   X₂ is an optional bond or optional second linker;
   X₃ is an optional bond or optional third linker;
   n is an integer ≥1;
   m is an integer ≥1.

Disclosed herein, in certain aspects, are pharmaceutical compositions comprising: a molecule obtained by any one of the methods disclosed herein and a pharmaceutically acceptable excipient. In some aspects, the pharmaceutical composition is formulated as a nanoparticle formulation. In some aspects, the pharmaceutical composition is formulated for parenteral, oral, intranasal, buccal, rectal, or transdermal administration.

Disclosed herein, in certain aspects, are kits comprising a molecule obtained by any one of the methods disclosed herein.

Disclosed herein, in certain aspects, are compositions comprising a polynucleic acid molecule conjugate, wherein the polynucleic acid molecule conjugate comprises a polynucleotide comprising a sequence having at least 60%, 65%, 70%, 75%, 80%, 85%, 90%, 95%, 96%, 97%, 98%, 99%, or 100% sequence identity to SEQ ID NOs: 54-972. Disclosed herein, in certain aspects, are compositions comprising a polynucleic acid molecule conjugate, wherein the polynucleic acid molecule conjugate comprises a polynucleotide comprising a sequence having at least 80%, 85%, 90%, 95%, 96%, 97%, 98%, 99%, or 100% sequence identity to SEQ ID NOs: 54-972. In certain aspects, the polynucleic acid molecule conjugate comprises a structure of Formula (I):

A-X-B Formula (I)
wherein,
A comprises a binding moiety;
B consists of the polynucleotide; and
X consists of a bond or first linker.

In certain aspects, the polynucleic acid molecule conjugate comprises a structure of Formula (II):

A-X-B-Y-C Formula (II)
wherein,
A comprises a binding moiety;
B consists of the polynucleotide;
C consists of a polymer;
X consists of a bond or first linker; and
Y consists of a bond or second linker.

In certain aspects, the polynucleic acid molecule conjugate comprises a structure of Formula (III):

A-X-C-Y-B Formula (III)
wherein,
A comprises a binding moiety;
B consists of the polynucleotide;
C consists of a polymer;
X consists of a bond or first linker; and
Y consists of a bond or second linker.

In certain aspects, the at least one 2′ modified nucleotide comprises a morpholino, 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleotide. In certain aspects, the at least one 2′ modified nucleotide comprises a morpholino.

Disclosed herein, in certain aspects, are compositions comprising antibody-peptide-oligonucleotide conjugate (APOC) or an antibody-peptide-polynucleic acid molecule conjugate, wherein the polynucleic acid molecule conjugate comprises a polynucleotide comprising a sequence having at least 60%, 65%, 70%, 75%, 80%, 85%, 90%, 95%, 96%, 97%, 98%, 99%, or 100% sequence identity to SEQ ID NOs: 54-972. Disclosed herein, in certain aspects, are compositions comprising or antibody-peptide-oligonucleotide conjugate (APOC) or an antibody-peptide-polynucleic acid molecule conjugate, wherein the antibody-peptide polynucleic acid molecule conjugate comprises a polynucleotide comprising a sequence having at least 80%, 85%, 90%, 95%, 96%, 97%, 98%, 99%, or 100% sequence identity to SEQ ID NOs: 54-972.

In some aspects, the antibody-peptide-oligonucleotide conjugate (APOC) or antibody-peptide-polynucleic acid molecule conjugate comprises a structure of Formula (V):

A-(X₁-B-X₂-D)n Formula (V)
wherein,
A is an antibody or antigen binding fragment thereof;
B is a polynucleotide;
D is an endosomolytic peptide or a membrane penetrating peptide;
X₁ is a bond or a first non-polymeric linker; and
X₂ is an optional bond or optional second linker;
n is an integer ≥1.

In some aspects, the antibody-peptide-oligonucleotide conjugate (APOC) or antibody-peptide-polynucleic acid molecule conjugate comprises a structure of Formula (VI):

A-(X₁-D-X₂-B)n Formula (VI)
wherein,
   A is an antibody or antigen binding fragment thereof;
   B is a polynucleotide;
   D is an endosomolytic peptide or a membrane penetrating peptide;
   X₁ is a bond or a first non-polymeric linker; and
   X₂ is an optional bond or optional second linker;
   n is an integer ≥1.

In some aspects, the antibody-peptide-polynucleic acid molecule conjugate or antibody-peptide-oligonucleotide conjugate comprises a structure of Formula (VII):

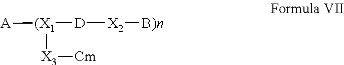

wherein,
   A is an antibody or antigen binding fragment thereof;
   B is a polynucleotide;
   D is an endosomolytic peptide or a membrane penetrating peptide
   C is a polymer;
   X₁ is a bond or a first non-polymeric linker;
   X₂ is an optional bond or optional second linker;
   X₃ is an optional bond or optional third linker;
   n is an integer ≥1;
   m is an integer ≥1.

Disclosed herein, in certain aspects, are methods of treating a disease or disorder comprising: administering to a subject a polynucleic acid molecule conjugate; wherein the polynucleic acid molecule conjugate comprises a target cell binding moiety and a targeted pre-mRNA specific splice modulating polynucleic acid moiety; wherein the target cell binding moiety specifically binds to a targeted cell, and the targeted pre-mRNA specific splice modulating polynucleic acid moiety induces insertion, deletion, duplication, or alteration of a targeted pre-mRNA transcript in the targeted cell to induce a splicing event in the targeted pre-mRNA transcript to generate a mRNA transcript; and wherein the mRNA transcript encodes a protein that is modified when compared to the same protein in untreated target cells, thereby treating the disease or disorder in the subject. In certain aspects, the splicing event is exon skipping. In certain aspects, the splicing event is exon inclusion. In certain aspects, the disease or disorder is further characterized by one or more mutations in the pre-mRNA. In certain aspects, the disease or disorder comprises a neuromuscular disease, a genetic disease, cancer, a hereditary disease, or a cardiovascular disease. In certain aspects, the disease or disorder is muscular dystrophy. In certain aspects, the disease or disorder is Duchenne muscular dystrophy. In certain aspects, the splicing event is of exon 8, 23, 35, 43, 44, 45, 50, 51, 52, 53, or 55 of DMD gene. In certain aspects, the splicing event is of exon 23 of DMD gene. In certain aspects, the splicing event is of an exon of PAH, MSTN, or K-Ras gene. In certain aspects, the polynucleic acid molecule conjugate comprises a structure of Formula (I):

A-X-B Formula (I)
wherein,
A comprises a binding moiety;
B consists of a polynucleotide; and
X consists of a bond or first linker.

In certain aspects, the polynucleic acid molecule conjugate comprises a structure of Formula (II):

A-X-B-Y-C Formula (II)
wherein,
A comprises a binding moiety;
B consists of a polynucleotide;
C consists of a polymer;
X consists a bond or first linker; and
Y consists of a bond or second linker.

In certain aspects, the polynucleic acid molecule conjugate comprises a structure of Formula (III):

A-X-C-Y-B Formula (III)
wherein,
A comprises a binding moiety;
B consists of a polynucleotide;
C consists of a polymer;
X consists of a bond or first linker; and
Y consists of a bond or second linker.

In certain aspects, the polynucleic acid molecule conjugate optionally comprises at least one 2′ modified nucleotide, at least one modified internucleotide linkage, or at least one inverted abasic moiety. In certain aspects, the at least one 2′ modified nucleotide comprises a morpholino, 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleotide. In certain aspects, the at least one 2′ modified nucleotide comprises locked nucleic acid (LNA), ethylene nucleic acid (ENA), or a peptide nucleic acid (PNA). In certain aspects, the at least one 2′ modified nucleotide comprises a morpholino. In certain aspects, the at least one inverted basic moiety is at least one terminus. In certain aspects, the at least one modified intemucleotide linkage comprises a phosphorothioate linkage or a phosphorodithioate linkage. In certain aspects, the polynucleic acid molecule comprises at least from about 10 to about 30 nucleotides in length. In certain aspects, the polynucleic acid molecule comprises at least about 15%, 20%, 30%, 40%, 50%, 60%, 70%, 80%, 90%, 95%, or 99% modification. In certain aspects, the polynucleic acid molecule comprises a single strand. In certain aspects, the polynucleic acid molecule comprises two or more strands. In certain aspects, the polynucleic acid molecule comprises a first polynucleotide and a second polynucleotide hybridized to the first polynucleotide to form a double-stranded polynucleic acid molecule. In certain aspects, the second polynucleotide comprises at least one modification. In certain aspects, the first polynucleotide and the second polynucleotide comprise RNA molecules. In certain aspects, the first polynucleotide and the second polynucleotide comprise siRNA molecules. In certain aspects, X is a bond. In certain aspects, X and Y are independently a bond, a degradable linker, a non-degradable linker, a cleavable linker, or a non-polymeric linker group. In certain aspects, X and Y are independently a bond, a degradable linker, a non-degradable linker, a cleavable linker, or a non-polymeric linker group. In certain aspects, X is a C₁-C₆ alkyl group. In certain aspects, X or Y is a C₁-C₆ alkyl group. In certain aspects, X or Y is a C₁-C₆ alkyl group. In certain aspects, the binding moiety is an antibody or binding fragment thereof. In certain aspects, the binding moiety is an antibody or binding fragment thereof. In certain aspects, the binding moiety is an antibody or binding fragment thereof. In certain aspects, C is polyethylene glycol. In certain aspects, C is polyethylene glycol. In certain aspects, A-X is conjugated to the 5′ end of B and Y—C is conjugated to the 3′ end of B. In certain aspects, Y—C is conjugated to the 5′ end of B and A-X is conjugated to the 3′ end of B. In certain aspects, methods further comprise D. In certain aspects, D is conjugated to C or to A. In certain aspects, methods further comprise at least a second binding moiety A. In certain aspects, methods further comprise at least a second binding moiety A. In certain aspects, methods further comprise at least a second binding moiety A.

Disclosed herein, in certain aspects, are methods of inducing a splicing event in a targeted pre-mRNA transcript, comprising: (a) contacting a target cell with a polynucleic acid molecule conjugate, wherein the polynucleic acid molecule conjugate comprises a target cell binding moiety and a targeted pre-mRNA splice modulating polynucleic acid moiety; (b) hybridizing the targeted pre-mRNA splice modulating polynucleic acid moiety to the targeted pre-mRNA transcript within the target cell to induce the splicing event in the targeted pre-mRNA transcript to produce a mRNA transcript; and (c) optionally, translating the mRNA transcript of step (b) in the target cell to produce a protein. In certain aspects, the splicing event is exon skipping. In certain aspects, the splicing event is exon inclusion. In certain aspects, the targeted pre-mRNA transcript induces a disease or disorder. In certain aspects, the disease or disorder comprises a neuromuscular disease, a genetic disease, cancer, a hereditary disease, or a cardiovascular disease. In certain aspects, the polynucleic acid molecule conjugate:
a) comprises a structure of Formula (I):

A-X-B Formula (I)
wherein,
A comprises a binding moiety;
B consists of the polynucleotide; and
X consists of a bond or first linker;
b) comprises a structure of Formula (II):

A-X-B-Y-C Formula (II)
wherein,
A comprises a binding moiety;
B consists of the polynucleotide;
C consists of a polymer;
X consists of a bond or first linker; and
Y consists of a bond or second linker; or
c) comprises a structure of Formula (III):

A-X-C-Y-B Formula (III)
wherein,
A comprises a binding moiety;
B consists of the polynucleotide;
C consists of a polymer;
X consists of a bond or first linker; and
Y consists of a bond or second linker.

In certain aspects, the polynucleic acid molecule conjugate optionally comprises at least one 2′ modified nucleotide, at least one modified internucleotide linkage, or at least one inverted abasic moiety. In certain aspects, the at least one 2′ modified nucleotide comprises a morpholino, 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleotide. In certain aspects, the at least one 2′ modified nucleotide comprises locked nucleic acid (LNA), ethylene nucleic acid (ENA), peptide nucleic acid (PNA). In certain aspects, the at least one 2′ modified nucleotide comprises a morpholino. In certain aspects, the at least one inverted basic moiety is at least one terminus. In certain aspects, the at least one modified intemucleotide linkage comprises a phosphorothioate linkage or a phosphorodithioate linkage. In certain aspects, the polynucleic acid molecule comprises at least from about 10 to about 30 nucleotides in length. In certain aspects, the polynucleic acid molecule comprises at least about 15%, 20%, 30%, 40%, 50%, 60%, 70%, 80%, 90%, 95%, or 99% modification. In certain aspects, the polynucleic acid molecule comprises at least about 3, about 4, about 5, about 6, about 7, about 8, about 9, about 10, about 11, about 12, about 13, about 14, about 15, about 16, about 17, about 18, about 19, about 20, about 21, about 22 or more modifications. In certain aspects, X and Y are independently a bond, a degradable linker, a non-degradable linker, a cleavable linker, or a non-polymeric linker group. In certain aspects, X is a bond. In certain aspects, X is a C1-C6 alkyl group. In certain aspects, Y is a C1-C6 alkyl group. In certain aspects, X is a homobifunctional linker or a heterobifunctional linker, optionally conjugated to a C1-C6 alkyl group. In certain aspects, Y is a homobifunctional linker or a heterobifunctional linker. In certain aspects, the binding moiety is an antibody or binding fragment thereof. In certain aspects, C is polyethylene glycol. In certain aspects, A-X is conjugated to the 5′ end of B and Y—C is conjugated to the 3′ end of B. In certain aspects, Y—C is conjugated to the 5′ end of B and A-X is conjugated to the 3′ end of B. In certain aspects, A-X, Y—C or a combination thereof is conjugated to an intemucleotide linkage group. In certain aspects, methods further comprise D. In certain aspects, D is conjugated to C or to A. In certain aspects, methods further comprise at least a second binding moiety A.

Disclosed herein, in certain aspects, are polynucleic acid molecule conjugate compositions comprising a target cell binding moiety and a targeted pre-mRNA specific splice modulating polynucleic acid moiety wherein the targeted pre-mRNA specific splice modulating polynucleic acid moiety comprises a sequence having at least 60%, 65%, 70%, 75%, 80%, 85%, 90%, 95%, 96%, 97%, 98%, 99%, or 100% sequence identity to SEQ ID NOs: 54-972. In certain aspects, the polynucleic acid molecule conjugate:
a) comprises a structure of Formula (I):

A-X-B Formula (I)
wherein,
A comprises a binding moiety;
B consists of the polynucleotide; and
X consists of a bond or first linker;
b) comprises a structure of Formula (II):

A-X-B-Y-C Formula (II)
wherein,
A comprises a binding moiety;
B consists of the polynucleotide;
C consists of a polymer;
X consists of a bond or first linker; and
Y consists of a bond or second linker; or
c) comprises a structure of Formula (III):

A-X-C-Y-B Formula (III)
wherein,
A comprises a binding moiety;
B consists of the polynucleotide;
C consists of a polymer;
X consists of a bond or first linker; and
Y consists of a bond or second linker.

In certain aspects, the pharmaceutical composition is formulated as a nanoparticle formulation.

Disclosed herein, in certain aspects, are the antibody-peptide-oligonucleotide conjugate (APOC) or antibody-peptide-polynucleic acid molecule conjugate compositions comprising a targeted pre-mRNA specific splice modulating polynucleic acid moiety wherein the targeted pre-mRNA specific splice modulating polynucleic acid moiety comprises a sequence having at least 60%, 65%, 70%, 75%, 80%, 85%, 90%, 95%, 96%, 97%, 98%, 99%, or 100% sequence identity to SEQ ID NOs: 54-972.

In some aspects, the antibody-peptide-oligonucleotide conjugate (APOC) or antibody-peptide-polynucleic acid molecule conjugate comprises a structure of Formula (V):

A-(X₁-B-X₂-D)n Formula (V)
wherein,
   A is an antibody or antigen binding fragment thereof;
   B is a polynucleotide;
   D is an endosomolytic peptide or a membrane penetrating peptide;
   X₁ is a bond or first non-polymeric linker; and
   X₂ is an optional bond or optional second linker;
   n is an integer ≥1.

In some aspects, the antibody-peptide-oligonucleotide conjugate (APOC) or antibody-peptide-polynucleic acid molecule conjugate comprises a structure of Formula (VI):

A-(X₁-D-X₂-B)n Formula (VI)
wherein,
   A is an antibody or antigen binding fragment thereof;
   B is a polynucleotide;
   D is an endosomolytic peptide or a membrane penetrating peptide;
   X₁ is a bond or first non-polymeric linker; and
   X₂ is an optional bond or optional second linker;
   n is an integer ≥1.

In some aspects, the antibody-peptide-polynucleic acid molecule conjugate or antibody-peptide-oligonucleotide conjugate comprises a structure of Formula (VII):

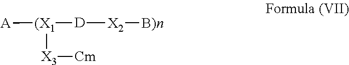

wherein,
   A is an antibody or antigen binding fragment thereof;
   B is a polynucleotide;
   D is an endosomolytic peptide or a membrane penetrating peptide
   C is a polymer;
   X₁ is a bond or first non-polymeric linker;
   X₂ is an optional bond or optional second linker;
   X₃ is an optional bond or optional third linker;
   n is an integer ≥1;
   m is an integer ≥1.

Disclosed herein, in certain aspects, is an antibody-peptide-oligonucleotide conjugate (APOC) comprising:

A-(X₁-B-X₂-D)n Formula (V)

or A-(X₁-D-X₂-B)n Formula (VI)
wherein,
   A is an antibody or antigen binding fragment thereof;
   B is a polynucleotide;
   D is an endosomolytic peptide or a membrane penetrating peptide;
   X₁ is a bond or a first non-polymeric linker;
   X₂ is an optional bond or an optional second linker; and
   n is an integer ≥1;
      wherein the polynucleotide comprises at least one 2′ modified nucleotide, at least one modified intemucleotide linkage, or at least one inverted abasic moiety. In some aspects, the antibody or antigen binding fragment thereof comprises a humanized antibody or antigen binding fragment thereof, chimeric antibody or antigen binding fragment thereof, monoclonal antibody or antigen binding fragment thereof, monovalent Fab′, divalent Fab2, single-chain variable fragment (scFv), diabody, minibody, nanobody, single-domain antibody (sdAb), or camelid antibody or antigen binding fragment thereof. In some aspects, the antibody or antigen binding fragment thereof binds to the transferrin receptor. In some aspects, D is an endosomolytic peptide. In some aspects, the endosomolytic peptide is selected from INF7 and melittin. In some aspects, D is a membrane penetrating peptide. In some aspects, the membrane penetrating peptide is selected from RRRRRRRRRRRR (SEQ ID NO:1000), GLAFLGFLGAAGSTMGAWSQPKKKRKV (SEQ ID NO:1001), RRIRPRPPRLPRPRPRPLPFPRPG (SEQ ID NO:1002), RKKRRQRRR (SEQ ID NO:1003), RRRRRRRRRR (SEQ ID NO:1004), GRPRESGKKRKRKRLKP (SEQ ID NO:1005), ALWKTLLKKVLKAPKKKRKV (SEQ ID NO:1006), RRIPNRRPRR (SEQ ID NO:1007), TRRQRTRRARRNR (SEQ ID NO:1008), HARIKPTFRRLKWKYKGKFW (SEQ ID NO:1009), GIGAVLKVLTTGLPALISWIKRKRQQ (SEQ ID NO:1010), LRRERQSRLRRERQSR (SEQ ID NO:1011), RRRRRRRRR (SEQ ID NO:1012), RQIKIWFQNRRMKWKK (SEQ ID NO:1013), KRARNTEAARRSRARKLQRMKQ (SEQ ID NO:1014), RHIKIWFQNRRMKWKK (SEQ ID NO:1015), RRRRRRRR (SEQ ID NO:1016), KMTRAQRRAAARRNRWTAR (SEQ ID NO:1017), RGGRLSYSRRRFSTSTGR (SEQ ID NO:1018), KQINNWFINQRKRHWK (SEQ ID NO:1019), KLWMRWYSPTTRRYG (SEQ ID NO:1020), RRWWRRWRR (SEQ ID NO:1021), SQIKIWFQNKRAKIKK (SEQ ID NO:1022), GAYDLRRRERQSRLRRRERQSR (SEQ ID NO:1023), TRRNKRNRIQEQLNRK (SEQ ID NO:1024), GKRKKKGKLGKKRDP (SEQ ID NO:1025), RQVTIWFQNRRVKEKK (SEQ ID NO:1026), RLRWR (SEQ ID NO:1027), PPRPPRPPRPPRPPR (SEQ ID NO:1028), CAYHRLRRC (SEQ ID NO:1029), SRRARRSPRHLGSG (SEQ ID NO:1030), PPRPPRPPRPPR (SEQ ID NO:1031), NAKTRRHERRRKLAIER (SEQ ID NO:1032), VKRGLKLRHVRPRVTRMDV (SEQ ID NO: 1033), LYKKGPAKKGRPPLRGWFH (SEQ ID NO:1034), TAKTRYKARRAELIAERR (SEQ ID NO:1035), KGTYKKKLMRIPLKGT (SEQ ID NO:1036), PPRPPRPPR (SEQ ID NO:1037), RASKRDGSWVKKLHRILE (SEQ ID NO:1038), TRSSRAGLQWPVGRVHRLLRK (SEQ ID NO:1039), FKIYDKKVRTRVVKH (SEQ ID NO:1040), VRLPPPVRLPPPVRLPPP (SEQ ID NO:1041), GPFHFYQFLFPPV (SEQ ID NO:1042), PLILLRLLRGQF (SEQ ID NO:1043), YTAIAWVKAFIRKLRK (SEQ ID NO:1044), KETWWETWWTEWSQPKKRKV (SEQ ID NO:1045), LIRLWSHLIHIWFQNRRLKWKKK (SEQ ID NO:1046), VDKGSYLPRPTPPRPIYNRN (SEQ ID NO:1047), MDAQTRRRERRAEKQAQWKAAN (SEQ ID NO:1048), GSPWGLQHHPPRT (SEQ ID NO:1049), KLALKALKALKAALKLA (SEQ ID NO:1050), IPALK (SEQ ID NO:1051), VPALR (SEQ ID NO:1052), LLIILRRRIRKQAHAHSK (SEQ ID NO:1053), IAWVKAFIRKLRKGPLG (SEQ ID NO:1054), AAVLLPVLLAAPVQRKRQKLP (SEQ ID NO:1055), TSPLNIHNGQKL (SEQ ID NO:1056), VPTLK (SEQ ID NO:1057), and VSALK (SEQ ID NO:1058), and (RXR)4XB (SEQ ID NO:1065), RXRRXRRXRRXRXB (SEQ ID NO:1066). In some aspects, the membrane penetrating peptide is RRRRRRRR (SEQ ID NO:1016), (RXR)4XB (SEQ ID NO:1065), or RXRRXRRXRRXRXB (SEQ ID NO: 1066). In some aspects, the membrane penetrating peptide is (RXR)4XB (SEQ ID NO:1065). In some aspects, D-X2 is conjugated to the 5′ end of B. In some aspects, D-X2 is conjugated to the 3′ end of B. In some aspects, the at least one 2′ modified nucleotide comprises 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2-O-dimethylaminopropyl (2′-O-DMAP), 2′-O-dimethylaminoethyloxyethyl (2′-O-DMAEOE), or 2′-O—N-methylacetamido (2′-O-NMA) modified nucleotide. In some aspects, the at least one 2′ modified nucleotide comprises locked nucleic acid (LNA) or ethylene nucleic acid (ENA). In some aspects, the at least one modified intemucleotide linkage comprises a phosphorothioate linkage or a phosphorodithioate linkage. In some aspects, the at least one inverted abasic moiety is at least one terminus. In some aspects, the polynucleotide comprises a single-stranded nucleotide. In some aspects, the single-stranded nucleotide comprises an antisense oligonucleotide (ASO) or phosphorodiamidate morpholino oligonucleotide (PMO). In some aspects, the polynucleotide comprises a first polynucleotide and a second polynucleotide hybridized to the first polynucleotide to form a double-stranded polynucleic acid molecule. In some aspects, the second polynucleotide comprises at least one modification. In some aspects, the first polynucleotide and the second polynucleotide are RNA molecules. In some aspects, the double-stranded polynucleic acid is a small interfering RNA (siRNA). In some aspects, the polynucleotide comprises a sequence having at least 90%, 95%, 99%, or 100% sequence identity to a sequence selected from SEQ ID NOs:225-227, 252-263, 268-272, 352-427, 768-827, 939-972. In some aspects, the polynucleotide comprises a sequence having least 90%, 95%, 99%, or 100% sequence identity to a sequence selected from SEQ ID NOs: 352-427 and 768-827. In some aspects, X₁ is a non-polymeric linker group. In some aspects, X₂ is a bond. In some aspects, X₂ is a C₁-C₆ alkyl group. In some aspects, X₂ is a homobifunctional linker or a heterobifunctional linker, optionally conjugated to a C₁-C₆ alkyl group. In some aspects, X₂ is a homobifunctional linker or a heterobifunctional linker. In some aspects, X₁ is a cleavable linker. In some aspects, the cleavable linker is a maleimide group with a-valine-citrulline linker. In some aspects, X₁ is a non-cleavable linker. In some aspects, non-cleavable linker is a maleimide group. In some aspects, the conjugate further comprises C, where C is a polymer. In some aspects, C is polyethylene glycol. In some aspects, C has a molecular weight of about 1000 Da, 2000 Da, or 5000 Da. In some aspects, C is conjugated to the molecule of Formula (VI) according to Formula (VII):

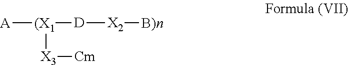

wherein,
   A is an antibody or antigen binding fragment thereof;
   B is a polynucleotide;
   D is an endosomolytic peptide or a membrane penetrating peptide
   C is a polymer;
   X₁ is a bond or first non-polymeric linker;
   X₂ is an optional bond or optional second linker;
   X₃ is a bond or third linker;
   n is an integer ≥1;
   m is an integer ≥1; and
      wherein the polynucleotide comprises at least one 2′ modified nucleotide, at least one modified intemucleotide linkage, or at least one inverted abasic moiety; wherein A and C are not attached to B at the same terminus; and wherein D is conjugated anywhere on A or C or to a terminus of B. In some aspects, X₃ is a C₁-C₆ alkyl group. In some aspects, X₃ is a homobifunctional linker or a heterobifunctional linker. In some aspects, described herein is a pharmaceutical composition comprising: an antibody-peptide-oligonucleotide conjugate described herein; and a pharmaceutically acceptable excipient. In some aspects, the pharmaceutical composition is formulated as a nanoparticle formulation. In some aspects, the pharmaceutical composition is formulated for parenteral, oral, intranasal, buccal, rectal, or transdermal administration. In some aspects, described herein is a method of treating a muscular dystrophy in a subject in need thereof, comprising: administering to the subject an antibody-peptide-oligonucleotide conjugate described herein; wherein the antibody-peptide-oligonucleotide conjugate induces splicing out of an exon to generate a mRNA transcript, and wherein the mRNA transcript encodes a truncated protein, thereby treating the muscular dystrophy in the subject. In some aspects, the muscular dystrophy is Duchenne muscular dystrophy. In some aspects, the splicing event is of exon 8, 23, 35, 43, 44, 45, 50, 51, 52, 53, or 55 of DMD gene. In some aspects, the splicing event is of exon 44 of DMD gene. In some aspects, the splicing event is of exon 45 of DMD gene. In some aspects, the splicing event is of exon 53 of DMD gene. In some aspects, the antibody or antigen binding fragments thereof is an anti-transferrin receptor antibody. In some aspects, the antibody or antigen binding fragments thereof is an anti-human transferrin receptor antibody. In some aspects, the antibody or antigen binding fragment thereof comprises a humanized antibody or antigen binding fragment thereof, chimeric antibody or antigen binding fragment thereof, monoclonal antibody or antigen binding fragment thereof, monovalent Fab′, divalent Fab2, single-chain variable fragment (scFv), diabody, minibody, nanobody, single-domain antibody (sdAb), or camelid antibody or antigen binding fragment thereof. In some aspects, the polynucleotide is an antisense oligonucleotide. In some aspects, the polynucleotide comprises at least from about 10 to about 30 nucleotides in length. In some aspects, the polynucleotide comprises one or more morpholino modifications. In some aspects, the polynucleotide is a morpholino antisense oligonucleotide. In some aspects, the polynucleotide comprises at least 90%, 95%, 99%, or 100% sequence identity to a sequence selected from SEQ ID NOs: 225-227, 252-263, 268-272, 352-427, 768-827, 939-972. In some aspects, the polynucleotide comprises at least 90%, 95%, 99%, or 100% sequence identity to a sequence selected from SEQ ID NOs: 352-427 and 768-827. In some aspects, the polynucleotide is conjugated to the antibody or antigen binding fragment thereof via a linker. In some aspects, the linker is a cleavable linker. In some aspects, the linker is a non-cleavable linker. In some aspects, the linker is selected from the group consisting of a heterobifunctional linker, a homobifunctional linker, a maleimide group, a dipeptide moiety, a benzoic acid group or derivatives thereof, a C₁-C₆ alkyl group, or a combination thereof. In some aspects, the antibody-peptide-oligonucleotide conjugate has a polynucleotide to antibody ratio of about 1:1, 2:1, 3:1, or 4:1. In some aspects, the subject is a human. In some aspects, described herein is method of inducing exon skipping in a subject in need thereof, comprising: administering to the subject an antibody-peptide-oligonucleotide conjugate described herein; wherein the antibody-peptide-oligonucleotide conjugate induces exon skipping in the pre-mRNA transcript to generate a mRNA transcript, and wherein the mRNA transcript encodes a truncated protein. In some aspects, described herein is a method of treating a muscular dystrophy in a subject in need thereof, comprising: administering to the subject an antibody-peptide-oligonucleotide conjugate described herein; wherein the antibody-peptide-oligonucleotide conjugate induces exon skipping in the pre-mRNA transcript to generate a mRNA transcript, and wherein the mRNA transcript encodes a truncated dystrophin protein, thereby treating the muscular dystrophy in the subject.

### DESCRIPTION OF THE DRAWINGS

FIG. 1 depicts a phosphorodiamidate morpholino oligomer (PMO) sequence with end nucleotides expanded (SEQ ID NO: 28).

FIG. 2A depicts a phosphorothioate antisense oligonucleotide (PS ASO) sequence with end nucleotides expanded (SEQ ID NO: 29).

FIG. 2B depicts a fully expanded phosphorothioate antisense oligonucleotide (PS ASO) sequence (SEQ ID NO: 29).

FIG. 3 depicts methods used to quantify skipped DMD mRNA in total RNA using Taqman qPCR.

FIG. 4 depicts a chromatogram of anti-CD71 mAb-PMO reaction mixture produced with hydrophobic interaction chromatography (HIC) method 2.

FIG. 5A depicts a chromatogram of anti-CD71 mAb produced using size exclusion chromatography (SEC) method 1.

FIG. 5B depicts a chromatogram of anti-CD71 mAb-PMO DAR 1,2 produced using size exclusion chromatography (SEC) method 1.

FIG. 5C depicts a chromatogram of anti-CD71 mAb-PMO DAR >2 produced using size exclusion chromatography (SEC) method 1.

FIG. 6A depicts a chromatogram of anti-CD71 mAb produced using hydrophobic interaction chromatography (HIC) method 2.

FIG. 6B depicts a chromatogram of purified anti-CD71 mAb-PMO DAR 1,2 conjugate produced using hydrophobic interaction chromatography (HIC) method 2.

FIG. 6C depicts a chromatogram of purified anti-CD71 mAb-PMO DAR >2 conjugate produced using hydrophobic interaction chromatography (HIC) method 2.

FIG. 7A depicts a chromatogram of fast protein liquid chromatography (FPLC) purification of anti-CD71 Fab-PMO using hydrophobic interaction chromatography (HIC) method 3.

FIG. 7B depicts a chromatogram of anti-CD71 Fab produced using SEC method 1.

FIG. 7C depicts a chromatogram of anti-CD71 Fab-PMO DAR 1 conjugate produced using SEC method 1.

FIG. 7D depicts a chromatogram of anti-CD71 Fab-PMO DAR 2 conjugate produced using SEC method 1.

FIG. 7E depicts a chromatogram of anti-CD71 Fab-PMO DAR 3 conjugate produced using SEC method 1.

FIG. 7F depicts a chromatogram of anti-CD71 Fab produced using HIC method 4.

FIG. 7G depicts a chromatogram of anti-CD71 Fab-PMO DAR 1 conjugate produced using HIC method 4.

FIG. 7H depicts a chromatogram of anti-CD71 Fab-PMO DAR 2 conjugate produced using HIC method 4.

FIG. 7I depicts a chromatogram of anti-CD71 Fab-PMO DAR 3 conjugate produced using HIC method 4.

FIG. 8A depicts a chromatogram of anti-CD71 mAb-PS ASO reaction mixture produced with SAX method 2.

FIG. 8B depicts a chromatogram of anti-CD71 mAb produced using SEC method 1.

FIG. 8C depicts a chromatogram of anti-CD71 mAb-PS ASO DAR 1 conjugate produced using SEC method 1.

FIG. 8D depicts a chromatogram of anti-CD71 mAb-PS ASO DAR 2 conjugate produced using SEC method 1.

FIG. 8E depicts a chromatogram of anti-CD71 mAb-PS ASO DAR 3 conjugate produced using SEC method 1.

FIG. 8F depicts a chromatogram of anti-CD71 mAb-PS ASO DAR 1 conjugate produced using SAX method 2.

FIG. 8G depicts a chromatogram of anti-CD71 mAb-PS ASO DAR 2 conjugate produced using SAX method 2.

FIG. 8H depicts a chromatogram of anti-CD71 mAb-PS ASO DAR 3 conjugate produced using SAX method 2.

FIG. 9 depicts an agarose gel from nested PCR detecting exon 23 skipping in differentiated C2C12 cells using PMO and anti-CD71 mAb-PMO conjugate.

FIG. 10 depicts an agarose gel from nested PCR detecting exon 23 skipping in differentiated C2C12 cells using PMO, anti-CD71 mAb-PMO, and anti-CD71 Fab-PMO conjugates.

FIG. 11 depicts an agarose gel from nested PCR detecting exon 23 skipping in differentiated C2C12 cells PMO, ASO, conjugated anti-CD71 mAb-ASO of DAR1 (“ASC-DAR1”), conjugated anti-CD71 mAb-ASO of DAR2 (“ASC-DAR2”), and conjugated anti-CD71 mAb-ASO of DAR3 (“ASC-DAR3”).

FIG. 12A depicts an agarose gel from nested PCR detecting exon 23 skipping in gastrocnemius muscle of wild-type mice administered a single intravenous injection of anti-CD71 mAb-PMO conjugate.

FIG. 12B is a graph of quantification of PCR products from gastrocnemius muscle.

FIG. 12C is a graph of quantification of in vivo exon skipping using Taqman qPCR from gastrocnemius muscle from wild-type mice.

FIG. 13A depicts an agarose gel from nested PCR detecting exon 23 skipping in heart muscle from wild-type mice after a single intravenous injection.

FIG. 13B is a graph of quantification of PCR products from heart muscle.

FIG. 14 depicts sequencing data of DNA fragments from skipped and wild-type PCR products (SEQ ID NOs: 976-977, respectively).

FIG. 15A is a graph of quantification of in vivo exon skipping in wild type mice in gastrocnemius muscle using Taqman qPCR.

FIG. 15B is a graph of quantification of in vivo exon skipping in wild type mice in gastrocnemius muscle using nested PCR.

FIG. 15C is a graph of quantification of in vivo exon skipping in wild type mice in diaphragm muscle using Taqman qPCR.

FIG. 15D is a graph of quantification of in vivo exon skipping in wild type mice in diaphragm muscle using nested PCR.

FIG. 15E is a graph of quantification of in vivo exon skipping in wild type mice in heart muscle using Tagman qPCR.

FIG. 15F is a graph of quantification of in vivo exon skipping in wild type mice in heart muscle using nested PCR.

FIG. 16A depicts an agarose gel from PCR detecting CD71 mAb-PMO conjugate induction of MSTN exon 2 skipping in diaphragm muscle tissues in wild type mice after a single intravenous (i.v.) injection.

FIG. 16B depicts an agarose gel from PCR detecting CD71 mAb-PMO conjugate induction of MSTN exon 2 skipping in heart muscle tissues in wild type mice after a single intravenous (i.v.) injection.

FIG. 16C depicts an agarose gel from PCR detecting CD71 mAb-PMO conjugate induction of MSTN exon 2 skipping in gastrocnemius muscle tissues in wild type mice after a single intravenous (i.v.) injection.

FIG. 17 depicts an agarose gel from PCR detecting ASGPR mAb-PMO conjugate induction of PAH exon 11 skipping in primary mouse hepatocytes.

FIG. 18 depicts an agarose gel from PCR detecting ASGPR mAb-PMO conjugate induction of PAH exon 11 skipping in livers from wild type mice after a single intravenous (i.v.) injection.

FIG. 19 depicts a SCX chromatogram of the PPMO product using SCX method 1.

FIG. 20 depicts a RP-PHLC chromatogram of PMO starting material (1) and PPMO product (2) using HPLC method 1.

FIG. 21 depicts a RP-HPLC of PPMO-DBCO-maleimide reaction showing the PPMO starting material (1) and the PPMO-sulfoDBCO-maleimide (2). Data was acquired using reversed-phase HPLC method 1.

FIG. 22 depicts a SCX chromatogram of the CD71 mAb-PPMO purification using SCX method 3.

FIG. 23 depicts an analysis of purified DAR 1.7 CD71 mAb-PPMO using SCX method 3.

FIG. 24 depicts a SCX chromatogram of the CD71 mAb-PPMO purification using SCX method 3.

FIG. 25 depicts an analysis of purified DAR 3.5 CD71 mAb-PPMO using SCX method 2.

FIG. 26 depicts a chromatogram of PPMO produced using SCX method 4.

FIG. 27 depicts a chromatogram of mAb-PPMO DAR1 produced using SCX method 4.

FIG. 28 depicts a HIC chromatogram of the CD71 mAb-PMO purification using HIC method 2.

FIG. 29 depicts an analysis of purified low DAR CD71 mAb-PMO using HIC method 1.

FIG. 30 depicts a SCX chromatogram of the CD71 mAb-PMO purification using SCX method 2.

FIG. 31 depicts an analysis of purified low DAR CD71 mAb-PMO using HIC method 1.

FIG. 32 depicts chromatogram of CD71 mAb-PMO reaction mixture produced with HIC method 3 showing free antibody peak (1), free PMO (2), DAR 1 (3), DAR 2 (4), DAR 3 (5), DAR>3 (6).

FIG. 33 depicts HIC chromatogram of the CD71 mAb-PMO purification using HIC method 4.

FIG. 34 depicts chromatogram of CD71 mAb and CD71-mAb-PMO DAR≥2 produced using SEC method 2.

FIG. 35 depicts chromatogram of purified CD71 mAb-PMO DAR≥2 conjugate produced using HIC method 4.

FIG. 36 depicts graphs of exon skipping (% of total dystrophin RNA) of exon 23 in mouse dystrophin vs treatment PMO concentration for C12C12 cells treated with PMO, PPMO, PMO-AOC or PPMO-AOC.

FIG. 37 depicts graphs of exon skipping (% of total dystrophin RNA) of exon 23 in mouse dystrophin vs treatment PMO concentration for C12C12 cells treated with PMO, PPMO, PMO-AOC or PPMO-AOC.

FIG. 38 depicts exon skipping in gastroc, TA, diaphragm, and heart at 14 days post dose. Note: CD-71 PPMO DAR 3.5, 50 mg/kg AB dose group was mistakenly taken down at 120 hours post dose.

FIG. 39 depicts exon skipping in gastroc 14 Days post dose.

FIG. 40 depicts PMO/PPMO tissue concentrations in gastroc, TA, heart, diaphragm, and liver 14 days post dose.

FIG. 41 depicts exon skipping efficiency. Group average exon 23 skipping (%) is plotted on the y-axis, while group average tissue concentration (nM) is plotted on the x-axis.

FIG. 42 depicts mouse exon 23 PMO/PPMO standard curves in various tissue homogenates, reflecting the same percentage of tissue homogenate in diluted samples (also shown in Tables 31-35).

FIG. 43A-FIG. 43L illustrate cartoon representations of molecules described herein.

FIG. 44 illustrates cartoon representation of antigen-peptide-oligonucleotide conjugate molecules described herein.

FIG. 45 illustrates general synthetic strategy used to synthesize future AOC-PPMOs (ADB).

FIG. 46 illustrates an example of the synthetic strategy to produce PPMO-antibody oligonucleotide conjugates using a sulfo-DBCO-maleimide linker.

FIG. 47 illustrates an example of the synthetic strategy to produce PMO-antibody oligonucleotide conjugates (PMO-AOCs) using a sulfo-DBCO-maleimide linker.

FIG. 48 depicts a SCX chromatogram of the PPMO product using SCX method 6 comparing the PMO starting material to the purified Fmoc-PPMO.

FIG. 49 depicts a RP-HPLC chromatogram of the Fmoc deprotection of Fmoc-(RXR)4XB-PM023 PPMO using reversed-phase HPLC (RP-HPLC) method 1 comparing the Fmoc-(RXR)4XB-PM023 PPMO starting material to the purified, deprotection NH2-(RXR)4XB-PM023 PPMO.

FIG. 50 depicts a RP-HPLC chromatogram comparing the starting material NH2-(RXR)4XB-PM023 PPMO and the unmodified PM023 to the reaction mixture containing the product MC-(RXR)4XB-PM023.

FIG. 51 depicts an analysis of the anti-mCD71 Ab-MC-(RXR)4XB-PM023 by strong cation exchange (SCX) chromatography method 7.

FIG. 52 depicts an analysis of the anti-mCD71 Ab-MC-(RXR)4XB-PM023 using size exclusion chromatography method 1.

FIGS. 53 A-H illustrate orientation 1 and orientation 2 of the PPMO-AOCs and the in vivo exon 23 skipping efficacies of PPMO-AOCs with orientations 1 and 2 in muscles of mdx mice administered with a PPMO dose of 3.3 and 10 mg/kg for the PPMO-AOC with orientation 1 and a PPMO dose of 5 mg/kg for the PPMO-AOC with orientation 2 at Day 14. FIGS. 53 A-B are schematic representations depicting the orientation 1 and orientation 2 of the PPMO-AOCs. FIGS. 53 C-D are bar graphs quantifying the percentage of exon 23 skipping in the gastrocnemius muscle of mdx mice administered with the PPMO-AOCs with orientations 1 and 2. FIGS. 53 E-F are bar graphs quantifying the percentage of exon 23 skipping in the diaphragm muscle of mdx mice administered with the PPMO-AOCs with orientations 1 and 2. FIGS. 53 G-H are bar graphs quantifying the percentage of exon 23 skipping in the heart muscle of mdx mice administered with the PPMO-AOCs with orientations 1 and 2.

FIGS. 54 A-F illustrate orientation 1 and orientation 2 of the PPMO-AOCs and exon 23 PMO concentrations in muscles of mdx mice administered with a PPMO dose of 3.3 and 10 mg/kg for the PPMO-AOC with orientation 1 and a PPMO dose of 5 mg/kg for the PPMO-AOC with orientation 2 at Day 14. FIGS. 54 A-B are schematic representations depicting the orientation 1 and orientation 2 of the PPMO-AOCs. FIGS. 54 C-D are bar graphs quantifying the exon 23 PMO concentrations in the gastrocnemius muscle of mdx mice administered with the PPMO-AOCs with orientations 1 and 2. FIGS. 54 E-F are bar graphs quantifying exon 23 PMO concentrations in the heart muscle of mdx mice administered with the PPMO-AOCs with orientations 1 and 2.

### DETAILED DESCRIPTION OF THE DISCLOSURE

Nucleic acid (e.g., RNAi) therapy is a targeted therapy with high selectivity and specificity. However, in some instances, nucleic acid therapy is also hindered by poor intracellular uptake, insufficient intracellular concentrations in target cells, and low efficacy. To address these issues, various modifications of the nucleic acid composition are explored, such as for example, novel linkers for better stabilizing and/or lower toxicity, optimization of binding moiety for increased target specificity and/or target delivery, and nucleic acid polymer modifications for increased stability and/or reduced off-target effect.

In some instances, one such area where oligonucleotide is used is for treating muscular dystrophy. Muscular dystrophy encompasses several diseases that affect the muscle. Duchenne muscular dystrophy is a severe form of muscular dystrophy and caused by mutations in the DMD gene. In some instances, mutations in the DMD gene disrupt the translational reading frame and results in non-functional dystrophin protein.

Described herein, in certain aspects, are methods and compositions relating nucleic acid therapy to induce an insertion, deletion, duplication, or alteration in an incorrectly spliced mRNA transcript to induce exon skipping or exon inclusion, which is used to restore the translational reading frame. In some aspects, also described herein include methods and compositions for treating a disease or disorder characterized by an incorrectly processed pre-mRNA transcript, in which after removal of an exon, the mRNA is capable of encoding a functional protein, thereby treating the disease or disorder. In additional aspects, described herein include pharmaceutical compositions and kits for treating the same.

### RNA Processing

RNA has a central role in regulation of gene expression and cell physiology. Proper processing of RNA is important for translational of functional protein. Alterations in RNA processing such as a result of incorrect splicing of RNA can result in disease. For example, mutations in a splice site causes exposure of a premature stop codon, a loss of an exon, or inclusion of an intron. In some instances, alterations in RNA processing results in an insertion, deletion, or duplication. In some instances, alterations in RNA processing results in an insertion, deletion, or duplication of an exon. Alterations in RNA processing, in some cases, results in an insertion, deletion, or duplication of an intron.

Alternative transcriptional or splicing events include, but are not limited to, exon skipping, alternative 3′ splice site selection, alternative 5′ splice site selection, intron retention, mutually exclusive exons, alternative promoter usage, and alternative polyadenylation. Splicing events, in some aspects, results in an insertion, deletion, or duplication of an exon, for example, by exon skipping or exon inclusion.

### Exon Skipping

Exon skipping is a form of RNA splicing. In some cases, exon skipping occurs when an exon is skipped over or is spliced out of the processed pre-mRNA. As a result of exon skipping, the processed pre-mRNA does not contain the skipped exon. In some instances, exon skipping results in expression of an altered product.

In some instances, antisense oligonucleotides (AONs) are used to induce exon skipping.

In some instances, AONs are short nucleic acid sequences that bind to specific mRNA or pre-mRNA sequences. For example, AONs bind splice sites or exonic enhancers. In some instances, binding of AONs to specific mRNA or pre-mRNA sequences generates double-stranded regions.

In some instances, formation of double-stranded regions occurs at sites where the spliceosome or proteins associated with the spliceosome would normally bind and causes exons to be skipped. In some instances, skipping of exons results in restoration of the transcript reading frame and allows for production of a partially functional protein.

### Exon Inclusion

In some instances, a mutation in RNA results in exon skipping. In some cases, a mutation is at least one of at the splice site, near the splice site, and at a distance from the splice site. In some instances, the mutations result in at least one of inactivating or weakening the splice site, disrupting exon splice enhancer or intron splice enhancer, and creating an exon splice silencer or intron splice enhancer. Mutations in some instances alter RNA secondary structure. In some cases, a mutation alters a RNA secondary structure result in disrupting the accessibility of signals important for exon recognition.

In some instances, use of AONs results in inclusion of the skipped exon. In some instances, the AONs bind to at least one of a splice site, a site near a splice site, and a site distant to a splice site. In some cases, AONs bind at site in the RNA to prevent disruption of an exon splice enhancer or intron splice enhancer. In some instances, AONs bind at site in the RNA to prevent creation of an exon splice silencer or intron splice silencer.

### Intron Retention

In some instances, a mutation in RNA results in intron retention. Intron retention results in an intron remaining in the mature mRNA transcript. In some instances, presence of a retained intron prevents or reduces translation of a functional protein. In some instances, intron retention occurs in a coding region, a non-coding region, at the 5′ UTR, or at the 3′ UTR. Where intron retention occurs in a coding region, in some instances, the retained intron encodes amino acids in frame, or is in misalignment which generates truncated proteins or non-functional proteins due to stop codon or frame shifts. In some instances, the intron is retained between two exons, located at the 5′ UTR, or located at the 3′ UTR.

In some instances, AONs are used to hybridize to a partially processed pre-mRNA to initiate removal of a retained intron. In some instances, the AONs hybridize to an intronic splicing enhancer or an intronic splicing silencer. In some instances, the AONs hybridize at or a distance from a 5′ splice site, 3′ splice site, branchpoint, polypyrimidine tract, an intron silencer site, a cryptic intron splice site, a pseudo splice site, or an intron enhancer of the intron. In some instances, the AONs hybridize to an internal region of the intron.

### Indications

In some aspects, a polynucleic acid molecule or a pharmaceutical composition described herein is used for the treatment of a disease or disorder characterized with a defective mRNA. In some aspects, a polynucleic acid molecule or a pharmaceutical composition described herein is used for the treatment of disease or disorder by inducing an insertion, deletion, duplication, or alteration in an incorrectly spliced mRNA transcript to induce a splicing event. In some aspects, the splicing event is exon skipping or exon inclusion. In some aspects, the splicing event is intron retention.

In some aspects, a polynucleic acid molecule or a pharmaceutical composition described herein is used for the treatment of disease or disorder by inducing an insertion, deletion, duplication, or alteration in an incorrectly spliced mRNA transcript to induce exon skipping or exon inclusion.

A large percentage of human protein-coding genes are alternatively spliced. In some instances, a mutation results in improperly spliced or partially spliced mRNA. For example, a mutation is in at least one of a splice site in a protein coding gene, a silencer or enhancer sequence, exonic sequences, or intronic sequences. In some instances, a mutation results in gene dysfunction. In some instances, a mutation results in a disease or disorder.

In some instances, a disease or disorder resulting from improperly spliced or partially spliced mRNA includes, but not limited to, a neuromuscular disease, a genetic disease, cancer, a hereditary disease, or a cardiovascular disease.

In some instances, genetic diseases or disorders include an autosomal dominant disorder, an autosomal recessive disorder, X-linked dominant disorder, X-linked recessive disorder, Y-linked disorder, mitochondrial disease, or multifactorial or polygenic disorder.

In some instances, cardiovascular disease such as hypercholesterolemia results from improperly spliced or partially spliced mRNA. In hypercholesterolemia, it has been shown that a single nucleotide polymorphism in exon 12 of the low density lipoprotein receptor (LDLR) promotes exon skipping.

In some instances, improperly spliced or partially spliced mRNA results in cancer. For example, improperly spliced or partially spliced mRNA affects cellular processes involved in cancer including, but not limited to, proliferation, motility, and drug response. In some instances is a solid cancer or a hematologic cancer. In some instances, the cancer is bladder cancer, lung cancer, brain cancer, melanoma, breast cancer, Non-Hodgkin lymphoma, cervical cancer, ovarian cancer, colorectal cancer, pancreatic cancer, esophageal cancer, prostate cancer, kidney cancer, skin cancer, leukemia, thyroid cancer, liver cancer, or uterine cancer.

Improperly spliced or partially spliced mRNA in some instances causes a neuromuscular disease or disorder. Exemplary neuromuscular diseases include muscular dystrophy such as Duchenne muscular dystrophy, Becker muscular dystrophy, facioscapulohumeral muscular dystrophy, congenital muscular dystrophy, or myotonic dystrophy. In some instances, muscular dystrophy is genetic. In some instances, muscular dystrophy is caused by a spontaneous mutation. Becker muscular dystrophy and Duchenne muscular dystrophy have been shown to involve mutations in the DMD gene, which encodes the protein dystrophin. Facioscapulohumeral muscular dystrophy has been shown to involve mutations in double homeobox, 4 (DUX4) gene.

In some instances, improperly spliced or partially spliced mRNA causes Duchenne muscular dystrophy. Duchenne muscular dystrophy results in severe muscle weakness and is caused by mutations in the DMD gene that abolishes the production of functional dystrophin. In some instances, Duchenne muscular dystrophy is a result of a mutation in an exon in the DMD gene. In some instances, Duchenne muscular dystrophy is a result of a mutation in at least one of exon 1, 2, 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78 and 79 in the DMD gene. In some instances, Duchenne muscular dystrophy is a result of a mutation in at least one of exon 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, and 63 in the DMD gene. In some instances, Duchenne muscular dystrophy is a result of a mutation in at least one of exon 8, 23, 35, 43, 44, 45, 50, 51, 52, 53, and 55 in the DMD gene. In some instances, multiple exons are mutated. For example, mutation of exons 48-50 is common in Duchenne muscular dystrophy patients. In some instances, Duchenne muscular dystrophy is a result of mutation of exon 51. In some instances, Duchenne muscular dystrophy is a result of mutation of exon 23. In some instances, a mutation involves a deletion of an exon. In some instances, a mutation involves a duplication of an exon. In some instances, a mutation involves a point mutation in an exon. For example, it has been shown that some patients have a nonsense point mutation in exon 51 of the DMD gene.

In some instances, a polynucleic acid molecule or a pharmaceutical composition described herein is used for the treatment of muscular dystrophy. In some instances, a polynucleic acid molecule or a pharmaceutical composition described herein is used for the treatment of Duchenne muscular dystrophy, Becker muscular dystrophy, facioscapulohumeral muscular dystrophy, congenital muscular dystrophy, or myotonic dystrophy. In some instances, a polynucleic acid molecule or a pharmaceutical composition described herein is used for the treatment of Duchenne muscular dystrophy.

### Polynucleic Acid Molecule

In some aspects, a polynucleic acid molecule described herein that induces an insertion, deletion, duplication, or alteration in an incorrectly spliced mRNA transcript to induce exon skipping or exon inclusion. In some instances, the polynucleic acid molecule restores the translational reading frame. In some instances, the polynucleic acid molecule results in a functional and truncated protein.

In some instances, a polynucleic acid molecule targets a mRNA sequence. In some instances, the polynucleic acid molecule targets a splice site. In some instances, the polynucleic acid molecule targets a cis-regulatory element. In some instances, the polynucleic molecule targets a trans-regulatory element. In some instances, the polynucleic acid molecule targets exonic splice enhancers or intronic splice enhancers. In some instances, the polynucleic acid molecule targets exonic splice silencers or intronic splice silencers.

In some instances, a polynucleic acid molecule targets a sequence found in introns or exons. For example, the polynucleic acid molecule targets a sequence found in an exon that mediates splicing of said exon. In some instances, the polynucleic acid molecule targets an exon recognition sequence. In some instances, the polynucleic acid molecule targets a sequence upstream of an exon. In some instances, the polynucleic acid molecule targets a sequence downstream of an exon.

As described above, a polynucleic acid molecule targets an incorrectly processed pre-mRNA transcript which results in a disease or disorder not limited to a neuromuscular disease, a genetic disease, cancer, a hereditary disease, or a cardiovascular disease.

In some instances, a polynucleic acid molecule targets an exon that is mutated in a gene that causes a disease or disorder. Exemplary diseases or disorders include, but are not limited to, familial dysautonomia (FD), spinal muscular atrophy (SMA), medium-chain acyl-CoA dehydrogenase (MCAD) deficiency, Hutchinson-Gilford progeria syndrome (HGPS), myotonic dystrophy type I (DM1), myotonic dystrophy type II (DM2), autosomal dominant retinitis pigmentosa (RP), Duchenne muscular dystrophy (DMD), microcephalic osteodysplastic primordial dwarfism type 1 (MOPD1) (Taybi-Linder syndrome (TALS)), frontotemporal dementia with parkinsonism-17 (FTDP-17), Fukuyama congenital muscular dystrophy (FCMD), amyotrophic lateral sclerosis (ALS), hypercholesterolemia, and cystic fibrosis (CF). Exemplary genes that are involved in the disease or disorder include, but are not limited to, IKBKAP, SMN2, MCAD, LMNA, DMPK, ZNF9, MAPT, FKTN, TDP-43, LDLR, CFTR, DMD, PAH, MSTN, and K-Ras. In some aspects, the gene is DMD, PAH, MSTN, or K-Ras.

In some instances, a polynucleic acid molecule described herein targets a region that is at the exon-intron junction of an exon of a gene that causes a disease or disorder. In some aspects, the gene is IKBKAP, SMN2, MCAD, LMNA, DMPK, ZNF9, MAPT, FKTN, TDP-43, LDLR, CFTR, DMD, PAH, MSTN, or K-Ras. In some aspects, a polynucleic acid molecule described herein targets a region that is at the exon-intron junction of exon 1, 2, or 3 of MSTN. In some aspects, a polynucleic acid molecule described herein targets a region that is at the exon-intron junction of exon 2 of MSTN. In some aspects, a polynucleic acid molecule described herein targets a region that is at the exon-intron junction of exon 1, 2, 3, 4, 5, 6, 7, 8, 9, 10, 11, 12, 13, 14, 15, 16, 17, 18, 19, 20, or 21 of PAH. In some aspects, a polynucleic acid molecule described herein targets a region that is at the exon-intron junction of exon 11 of PAH.

In some instances, the polynucleic acid molecule hybridizes to a target region that is at either the 5′ intron-exon junction or the 3′ exon-intron junction of at least one of an exon of a gene that causes a disease or disorder. In some aspects, the gene is IKBKAP, SMN2, MCAD, LMNA, DMPK, ZNF9, MAPT, FKTN, TDP-43, LDLR, CFTR, DMD, PAH, MSTN, or K-Ras.

In some aspects, a polynucleic acid molecule described herein targets either the 5′ intron-exon junction or the 3′ exon-intron junction of exon 1, 2, or 3 of MSTN. In some aspects, a polynucleic acid molecule described herein targets a region that is either the 5′ intron-exon junction or the 3′ exon-intron junction of exon 2 of MSTN. In some aspects, a polynucleic acid molecule described herein targets a region that is either the 5′ intron-exon junction or the 3′ exon-intron junction of exon 1, 2, 3, 4, 5, 6, 7, 8, 9, 10, 11, 12, 13, 14, 15, 16, 17, 18, 19, 20, or 21 of PAH. In some aspects, a polynucleic acid molecule described herein targets a region that is either the 5′ intron-exon junction or the 3′ exon-intron junction of exon 11 of PAH.

In some cases, the polynucleic acid molecule hybridizes to a target region that is at the 5′ intron-exon junction of at least one of exon of a gene that causes a disease or disorder. In some aspects, the gene is IKBKAP, SMN2, MCAD, LMNA, DMPK, ZNF9, MAPT, FKTN, TDP-43, LDLR, CFTR, DMD, PAH, MSTN, or K-Ras. In some aspects, a polynucleic acid molecule described herein targets a region that is at the 5′ intron-exon junction of exon 1, 2, or 3 of MSTN.

In some aspects, a polynucleic acid molecule described herein targets a region that is at the 5′ intron-exon junction of exon 2 of MSTN. In some aspects, a polynucleic acid molecule described herein targets a region that is at the 5′ intron-exon junction of exon 1, 2, 3, 4, 5, 6, 7, 8, 9, 10, 11, 12, 13, 14, 15, 16, 17, 18, 19, 20, or 21 of PAH. In some aspects, a polynucleic acid molecule described herein targets a region that is at the 5′ intron-exon junction of exon 11 of PAH.

In some cases, the polynucleic acid molecule hybridizes to a target region that is at the 3′ exon-intron junction of at least one of exon of a gene that causes a disease or disorder. In some aspects, the gene is IKBKAP, SMN2, MCAD, LMNA, DMPK, ZNF9, MAPT, FKTN, TDP-43, LDLR, CFTR, DMD, PAH, MSTN, or K-Ras. In some aspects, a polynucleic acid molecule described herein targets a region that is at the 3′ exon-intron junction of exon 1, 2, or 3 of MSTN.

In some aspects, a polynucleic acid molecule described herein targets a region that is at the 3′ exon-intron junction of exon 2 of MSTN. In some aspects, a polynucleic acid molecule described herein targets a region that is at the 3′ exon-intron junction of exon 1, 2, 3, 4, 5, 6, 7, 8, 9, 10, 11, 12, 13, 14, 15, 16, 17, 18, 19, 20, or 21 of PAH. In some aspects, a polynucleic acid molecule described herein targets a region that is at the 3′ exon-intron junction of exon 11 of PAH.

In some cases, the polynucleic acid molecule described herein targets a splice site of an exon of a gene that causes a disease or disorder. In some aspects, the gene is IKBKAP, SMN2, MCAD, LMNA, DMPK, ZNF9, MAPT, FKTN, TDP-43, LDLR, CFTR, DMD, PAH, MSTN, or K-Ras. In some aspects, a polynucleic acid molecule described herein targets a splice site of exon 1, 2, or 3 of MSTN. In some aspects, a polynucleic acid molecule described herein targets a splice site of exon 2 of MSTN. In some aspects, a polynucleic acid molecule described herein targets a splice site of exon 1, 2, 3, 4, 5, 6, 7, 8, 9, 10, 11, 12, 13, 14, 15, 16, 17, 18, 19, 20, or 21 of PAH. In some aspects, a polynucleic acid molecule described herein targets a splice site of exon 11 of PAH. As used herein, a splice site includes a canonical splice site, a cryptic splice site or an alternative splice site that is capable of inducing an insertion, deletion, duplication, or alteration in an incorrectly spliced mRNA transcript to induce exon skipping or exon inclusion.

In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt upstream (or from the 5′) of an exon of a gene that causes a disease or disorder. In some aspects, the gene is IKBKAP, SMN2, MCAD, LMNA, DMPK, ZNF9, MAPT, FKTN, TDP-43, LDLR, CFTR, DMD, PAH, MSTN, or K-Ras. In some instances, a polynucleic acid molecule described herein targets a region at least 1000 nt, 500 nt, 400 nt, 300 nt, 200 nt, 100 nt, 80 nt, 60 nt, 50 nt, 40 nt, 30 nt, 20 nt, 10 nt, or 5 nt upstream (or from the 5′) of exon 1, 2, or 3 of the MSTN gene. In some instances, a polynucleic acid molecule described herein targets a region at least 1000 nt, 500 nt, 400 nt, 300 nt, 200 nt, 100 nt, 80 nt, 60 nt, 50 nt, 40 nt, 30 nt, 20 nt, 10 nt, or 5 nt upstream (or from the 5′) of exon 2 of the MSTN gene. In some instances, a polynucleic acid molecule described herein targets a region at least 1000 nt, 500 nt, 400 nt, 300 nt, 200 nt, 100 nt, 80 nt, 60 nt, 50 nt, 40 nt, 30 nt, 20 nt, 10 nt, or 5 nt upstream (or from the 5′) of exon 1, 2, 3, 4, 5, 6, 7, 8, 9, 10, 11, 12, 13, 14, 15, 16, 17, 18, 19, 20, or 21 of PAH gene. In some instances, a polynucleic acid molecule described herein targets a region at least 1000 nt, 500 nt, 400 nt, 300 nt, 200 nt, 100 nt, 80 nt, 60 nt, 50 nt, 40 nt, 30 nt, 20 nt, 10 nt, or 5 nt upstream (or from the 5′) of exon 11 of the PAH gene.

In some instances, the polynucleic acid molecule hybridizes to a target region that is upstream (or 5′) to at least one of an exon of a gene that causes a disease or disorder. In some aspects, the gene is IKBKAP, SMN2, MCAD, LMNA, DMPK, ZNF9, MAPT, FKTN, TDP-43, LDLR, CFTR, DMD, PAH, MSTN, or K-Ras. In some instances, the polynucleic acid molecule hybridizes to a target region that is upstream (or 5′) to at least one of exon 1, 2, or 3 of the MSTN gene. In some instances, the polynucleic acid molecule hybridizes to a target region that is about 5, 10, 15, 20, 50, 100, 200, 300, 400 or 500 bp upstream (or 5′) to at least one of exon 2 of the MSTN gene. In some instances, the polynucleic acid molecule hybridizes to a target region that is upstream (or 5′) to at least one of exon 1, 2, 3, 4, 5, 6, 7, 8, 9, 10, 11, 12, 13, 14, 15, 16, 17, 18, 19, 20, or 21 of the PAH gene. In some instances, the polynucleic acid molecule hybridizes to a target region that is about 5, 10, 15, 20, 50, 100, 200, 300, 400 or 500 bp upstream (or 5′) to at least one of exon 11 of the PAH gene.

In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt downstream (or from the 3′) of an exon of a gene that causes a disease or disorder.

In some aspects, the gene is IKBKAP, SMN2, MCAD, LMNA, DMPK, ZNF9, MAPT, FKTN, TDP-43, LDLR, CFTR, DMD, PAH, MSTN, or K-Ras. In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt downstream (or from the 3′) of exon 1, 2, or 3 of the MSTN gene. In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt downstream (or from the 3′) of exon 2 of the MSTN gene. In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt downstream (or from the 3′) of exon 1, 2, 3, 4, 5, 6, 7, 8, 9, 10, 11, 12, 13, 14, 15, 16, 17, 18, 19, 20, or 21 of the PAH gene. In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt downstream (or from the 3′) of exon 11 of the PAH gene.

In some instances, the polynucleic acid molecule hybridizes to a target region that is downstream (or 3′) to at least one of an exon of a gene that causes a disease or disorder. In some aspects, the gene is IKBKAP, SMN2, MCAD, LMNA, DMPK, ZNF9, MAPT, FKTN, TDP-43, LDLR, CFTR, DMD, PAH, MSTN, or K-Ras. In some instances, the polynucleic acid molecule hybridizes to a target region that is about 5, 10, 15, 20, 50, 100, 200, 300, 400 or 500 bp downstream (or 3′) to at least one of exon 1, 2, or 3 of the MSTN gene. In some instances, the polynucleic acid molecule hybridizes to a target region that is about 5, 10, 15, 20, 50, 100, 200, 300, 400 or 500 bp downstream (or 3′) to at least one of exon 2 of the MSTN gene. In some instances, the polynucleic acid molecule hybridizes to a target region that is about 5, 10, 15, 20, 50, 100, 200, 300, 400 or 500 bp downstream (or 3′) to at least one of exon 1, 2, 3, 4, 5, 6, 7, 8, 9, 10, 11, 12, 13, 14, 15, 16, 17, 18, 19, 20, or 21 of the PAH gene. In some instances, the polynucleic acid molecule hybridizes to a target region that is about 5, 10, 15, 20, 50, 100, 200, 300, 400 or 500 bp downstream (or 3′) to at least one of exon 11 of the PAH gene.

In some instances, a polynucleic acid molecule described herein targets an internal region within an exon of a gene that causes a disease or disorder. In some aspects, the gene is IKBKAP, SMN2, MCAD, LMNA, DMPK, ZNF9, MAPT, FKTN, TDP-43, LDLR, CFTR, DMD, PAH, MSTN, or K-Ras. In some instances, a polynucleic acid molecule described herein targets an internal region within exon 1, 2, or 3 of the MSTN gene. In some instances, a polynucleic acid molecule described herein targets an internal region within exon 2 of the MSTN gene. In some instances, a polynucleic acid molecule described herein targets an internal region within 1, 2, 3, 4, 5, 6, 7, 8, 9, 10, 11, 12, 13, 14, 15, 16, 17, 18, 19, 20, or 21 of the PAH gene. In some instances, a polynucleic acid molecule described herein targets an internal region within exon 11 of the PAH gene.

In some cases, a polynucleic acid molecule targets an incorrectly processed pre-mRNA transcript which results in a neuromuscular disease or disorder. In some cases, a neuromuscular disease or disorder is Duchenne muscular dystrophy, Becker muscular dystrophy, facioscapulohumeral muscular dystrophy, congenital muscular dystrophy, or myotonic dystrophy. In some cases, a polynucleic acid molecule targets an incorrectly processed pre-mRNA transcript which results in Duchenne muscular dystrophy, Becker muscular dystrophy, facioscapulohumeral muscular dystrophy, congenital muscular dystrophy, or myotonic dystrophy. In some cases, a polynucleic acid molecule targets an incorrectly processed pre-mRNA transcript which results in Duchenne muscular dystrophy.

In some instances, a polynucleic acid molecule targets an exon that is mutated in the DMD gene that causes Duchenne muscular dystrophy. Exemplary exons that are mutated in the DMD gene that causes Duchenne muscular dystrophy include, but not limited to, exon 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, and 63. In some instances, the polynucleic acid molecule targets a sequence adjacent to a mutated exon. For example, if there is a deletion of exon 50, the polynucleic acid molecule targets a sequence in exon 51 so that exon 51 is skipped. In another instance, if there is a mutation in exon 23, the polynucleic acid molecule targets a sequence in exon 22 so that exon 23 is skipped.

In some instances, a polynucleic acid molecule described herein targets a region that is at the exon-intron junction of exon 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 63 of the DMD gene. In some instances, a polynucleic acid molecule described herein targets a region that is at the exon-intron junction of exon 8, 23, 35, 43, 44, 45, 50, 51, 52, 53, or 55 of the DMD gene. In some cases, a polynucleic acid molecule described herein targets a region that is at the exon-intron junction of exon 8 of the DMD gene. In some cases, a polynucleic acid molecule described herein targets a region that is at the exon-intron junction of exon 23 of the DMD gene. In some cases, a polynucleic acid molecule described herein targets a region that is at the exon-intron junction of exon 35 of the DMD gene. In some cases, a polynucleic acid molecule described herein targets a region that is at the exon-intron junction of exon 43 of the DMD gene. In some cases, a polynucleic acid molecule described herein targets a region that is at the exon-intron junction of exon 44 of the DMD gene. In some cases, a polynucleic acid molecule described herein targets a region that is at the exon-intron junction of exon 45 of the DMD gene. In some cases, a polynucleic acid molecule described herein targets a region that is at the exon-intron junction of exon 48 of the DMD gene. In some cases, a polynucleic acid molecule described herein targets a region that is at the exon-intron junction of exon 49 of the DMD gene. In some cases, a polynucleic acid molecule described herein targets a region that is at the exon-intron junction of exon 50 of the DMD gene. In some cases, a polynucleic acid molecule described herein targets a region that is at the exon-intron junction of exon 51 of the DMD gene. In some cases, a polynucleic acid molecule described herein targets a region that is at the exon-intron junction of exon 52 of the DMD gene. In some cases, a polynucleic acid molecule described herein targets a region that is at the exon-intron junction of exon 53 of the DMD gene. In some cases, a polynucleic acid molecule described herein targets a region that is at the exon-intron junction of exon 55 of the DMD gene.

In some instances, the polynucleic acid molecule hybridizes to a target region that is at either the 5′ intron-exon junction or the 3′ exon-intron junction of at least one of exon 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 52, 53, 54, 55, 56, 57, 58, 59, 60, 61, 62, and 63 of the DMD gene.

In some instances, the polynucleic acid molecule hybridizes to a target region that is at either the 5′ intron-exon junction or the 3′ exon-intron junction of exon 8, 23, 35, 43, 44, 45, 50, 51, 52, 53, or 55 of the DMD gene.

In some cases, the polynucleic acid molecule hybridizes to a target region that is at the 5′ intron-exon junction of at least one of exon 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 52, 53, 54, 55, 56, 57, 58, 59, 60, 61, 62, and 63 of the DMD gene. In some cases, the polynucleic acid molecule hybridizes to a target region that is at the 5′ intron-exon junction of exon 8, 23, 35, 43, 44, 45, 50, 51, 52, 53, or 55 of the DMD gene. In some cases, the polynucleic acid molecule hybridizes to a target region that is at the 5′ intron-exon junction of exon 8 of the DMD gene. In some cases, the polynucleic acid molecule hybridizes to a target region that is at the 5′ intron-exon junction of exon 23 of the DMD gene. In some cases, the polynucleic acid molecule hybridizes to a target region that is at the 5′ intron-exon junction of exon 35 of the DMD gene. In some cases, the polynucleic acid molecule hybridizes to a target region that is at the 5′ intron-exon junction of exon 43 of the DMD gene. In some cases, the polynucleic acid molecule hybridizes to a target region that is at the 5′ intron-exon junction of exon 44 of the DMD gene. In some cases, the polynucleic acid molecule hybridizes to a target region that is at the 5′ intron-exon junction of exon 45 of the DMD gene. In some cases, the polynucleic acid molecule hybridizes to a target region that is at the 5′ intron-exon junction of exon 50 of the DMD gene. In some cases, the polynucleic acid molecule hybridizes to a target region that is at the 5′ intron-exon junction of exon 51 of the DMD gene. In some cases, the polynucleic acid molecule hybridizes to a target region that is at the 5′ intron-exon junction of exon 52 of the DMD gene. In some cases, the polynucleic acid molecule hybridizes to a target region that is at the 5′ intron-exon junction of exon 53 of the DMD gene. In some cases, the polynucleic acid molecule hybridizes to a target region that is at the 5′ intron-exon junction of exon 55 of the DMD gene.

In some cases, the polynucleic acid molecule hybridizes to a target region that is at the 3′ exon-intron junction of at least one of exon 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 52, 53, 54, 55, 56, 57, 58, 59, 60, 61, 62, and 63 of the DMD gene. In some cases, the polynucleic acid molecule hybridizes to a target region that is at the 3′ exon-intron junction of exon 8, 23, 35, 43, 44, 45, 50, 51, 52, 53, or 55 of the DMD gene. In some cases, the polynucleic acid molecule hybridizes to a target region that is at the 3′ exon-intron junction of exon 8 of the DMD gene. In some cases, the polynucleic acid molecule hybridizes to a target region that is at the 3′ exon-intron junction of exon 23 of the DMD gene. In some cases, the polynucleic acid molecule hybridizes to a target region that is at the 3′ exon-intron junction of exon 35 of the DMD gene. In some cases, the polynucleic acid molecule hybridizes to a target region that is at the 3′ exon-intron junction of exon 43 of the DMD gene. In some cases, the polynucleic acid molecule hybridizes to a target region that is at the 3′ exon-intron junction of exon 44 of the DMD gene. In some cases, the polynucleic acid molecule hybridizes to a target region that is at the 3′ exon-intron junction of exon 45 of the DMD gene. In some cases, the polynucleic acid molecule hybridizes to a target region that is at the 3′ exon-intron junction of exon 50 of the DMD gene. In some cases, the polynucleic acid molecule hybridizes to a target region that is at the 3′ exon-intron junction of exon 51 of the DMD gene. In some cases, the polynucleic acid molecule hybridizes to a target region that is at the 3′ exon-intron junction of exon 52 of the DMD gene. In some cases, the polynucleic acid molecule hybridizes to a target region that is at the 3′ exon-intron junction of exon 53 of the DMD gene. In some cases, the polynucleic acid molecule hybridizes to a target region that is at the 3′ exon-intron junction of exon 55 of the DMD gene.

In some instances, a polynucleic acid molecule described herein targets a splice site of exon 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 63 of the DMD gene. In some instances, a polynucleic acid molecule described herein targets a splice site of exon 8, 23, 35, 43, 44, 45, 50, 51, 52, 53, or 55 of the DMD gene. In some cases, a polynucleic acid molecule described herein targets a splice site of exon 8 of the DMD gene. In some instances, a polynucleic acid molecule described herein targets a splice site of exon 23 of the DMD gene. In some cases, a polynucleic acid molecule described herein targets a splice site of exon 35 of the DMD gene. In some cases, a polynucleic acid molecule described herein targets a splice site of exon 43 of the DMD gene. In some cases, a polynucleic acid molecule described herein targets a splice site of exon 44 of the DMD gene. In some cases, a polynucleic acid molecule described herein targets a splice site of exon 45 of the DMD gene.

In some instances, a polynucleic acid molecule described herein targets a splice site of exon 48 of the DMD gene. In some instances, a polynucleic acid molecule described herein targets a splice site of exon 49 of the DMD gene. In some instances, a polynucleic acid molecule described herein targets a splice site of exon 50 of the DMD gene. In some instances, a polynucleic acid molecule described herein targets a splice site of exon 51 of the DMD gene. In some cases, a polynucleic acid molecule described herein targets a splice site of exon 52 of the DMD gene. In some cases, a polynucleic acid molecule described herein targets a splice site of exon 53 of the DMD gene. In some cases, a polynucleic acid molecule described herein targets a splice site of exon 55 of the DMD gene. As used herein, a splice site includes a canonical splice site, a cryptic splice site or an alternative splice site that is capable of inducing an insertion, deletion, duplication, or alteration in an incorrectly spliced mRNA transcript to induce exon skipping or exon inclusion.

In some aspects, a polynucleic acid molecule described herein target a partially spliced mRNA sequence comprising additional exons involved in Duchenne muscular dystrophy such as exon 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 52, 53, 54, 55, 56, 57, 58, 59, 60, 61, 62, or 63.

In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt upstream (or from the 5′) of exon 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 63 of the DMD gene. In some instances, a polynucleic acid molecule described herein targets a region at least 1000 nt, 500 nt, 400 nt, 300 nt, 200 nt, 100 nt, 80 nt, 60 nt, 50 nt, 40 nt, 30 nt, 20 nt, 10 nt, or 5 nt upstream (or from the 5′) of exon 8, 23, 35, 43, 44, 45, 50, 51, 52, 53, or 55 of the DMD gene. In some instances, a polynucleic acid molecule described herein targets a region at least 1000 nt, 500 nt, 400 nt, 300 nt, 200 nt, 100 nt, 80 nt, 60 nt, 50 nt, 40 nt, 30 nt, 20 nt, 10 nt, or 5 nt upstream (or from the 5′) of exon 8 of the DMD gene. In some instances, a polynucleic acid molecule described herein targets a region at least 1000 nt, 500 nt, 400 nt, 300 nt, 200 nt, 100 nt, 80 nt, 60 nt, 50 nt, 40 nt, 30 nt, 20 nt, 10 nt, or 5 nt upstream (or from the 5′) of exon 23 of the DMD gene. In some instances, a polynucleic acid molecule described herein targets a region at least 1000 nt, 500 nt, 400 nt, 300 nt, 200 nt, 100 nt, 80 nt, 60 nt, 50 nt, 40 nt, 30 nt, 20 nt, 10 nt, or 5 nt upstream (or from the 5′) of exon 35 of the DMD gene. In some instances, a polynucleic acid molecule described herein targets a region at least 1000 nt, 500 nt, 400 nt, 300 nt, 200 nt, 100 nt, 80 nt, 60 nt, 50 nt, 40 nt, 30 nt, 20 nt, 10 nt, or 5 nt upstream (or from the 5′) of exon 43 of the DMD gene. In some instances, a polynucleic acid molecule described herein targets a region at least 1000 nt, 500 nt, 400 nt, 300 nt, 200 nt, 100 nt, 80 nt, 60 nt, 50 nt, 40 nt, 30 nt, 20 nt, 10 nt, or 5 nt upstream (or from the 5′) of exon 44 of the DMD gene. In some instances, a polynucleic acid molecule described herein targets a region at least 1000 nt, 500 nt, 400 nt, 300 nt, 200 nt, 100 nt, 80 nt, 60 nt, 50 nt, 40 nt, 30 nt, 20 nt, 10 nt, or 5 nt upstream (or from the 5′) of exon 45 of the DMD gene. In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt upstream (or from the 5′) of exon 48 of the DMD gene. In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt upstream (or from the 5′) of exon 49 of the DMD gene. In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt upstream (or from the 5′) of exon 50 of the DMD gene. In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt upstream (or from the 5′) of exon 51 of the DMD gene. In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt upstream (or from the 5′) of exon 52 of the DMD gene. In some instances, a polynucleic acid molecule described herein targets a region at least 1000 nt, 500 nt, 400 nt, 300 nt, 200 nt, 100 nt, 80 nt, 60 nt, 50 nt, 40 nt, 30 nt, 20 nt, 10 nt, or 5 nt upstream (or from the 5′) of exon 53 of the DMD gene.

In some instances, a polynucleic acid molecule described herein targets a region at least 1000 nt, 500 nt, 400 nt, 300 nt, 200 nt, 100 nt, 80 nt, 60 nt, 50 nt, 40 nt, 30 nt, 20 nt, 10 nt, or 5 nt upstream (or from the 5′) of exon 55 of the DMD gene.

In some instances, the polynucleic acid molecule hybridizes to a target region that is upstream (or 5′) to at least one of exon 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 52, 53, 54, 55, 56, 57, 58, 59, 60, 61, 62, and 63 of the DMD gene. In some instances, the polynucleic acid molecule hybridizes to a target region that is upstream (or 5′) to at least one of exon 8, 23, 35, 43, 44, 45, 50, 51, 52, 53, or 55 of the DMD gene. In some instances, the polynucleic acid molecule hybridizes to a target region that is about 5, 10, 15, 20, 50, 100, 200, 300, 400 or 500 bp upstream (or 5′) to at least one of exon 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 52, 53, 54, 55, 56, 57, 58, 59, 60, 61, 62, and 63 of the DMD gene.

In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt downstream (or from the 3′) of exon 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 63 of the DMD gene. In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt downstream (or from the 3′) of exon 8, 23, 35, 43, 44, 45, 50, 51, 52, 53, or 55 of the DMD gene. In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt downstream (or from the 3′) of exon 8 of the DMD gene. In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt downstream (or from the 3′) of exon 23 of the DMD gene. In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt downstream (or from the 3′) of exon 35 of the DMD gene. In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt downstream (or from the 3′) of exon 43 of the DMD gene. In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt downstream (or from the 3′) of exon 44 of the DMD gene. In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt downstream (or from the 3′) of exon 45 of the DMD gene.

In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt downstream (or from the 3′) of exon 48 of the DMD gene. In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt downstream (or from the 3′) of exon 49 of the DMD gene. In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt downstream (or from the 3′) of exon 50 of the DMD gene. In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt downstream (or from the 3′) of exon 51 of the DMD gene. In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt downstream (or from the 3′) of exon 52 of the DMD gene. In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt downstream (or from the 3′) of exon 53 of the DMD gene. In some instances, a polynucleic acid molecule described herein targets a region at least 1000 nucleotides (nt), 500 nt, 400 nt, 300 nt, 200 nt, 100 nt, 80 nt, 60 nt, 50 nt, 40 nt, 30 nt, 20 nt, 10 nt, or 5 nt downstream (or from the 3′) of exon 55 of the DMD gene.

In some instances, the polynucleic acid molecule hybridizes to a target region that is downstream (or 3′) to at least one of exon 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 52, 53, 54, 55, 56, 57, 58, 59, 60, 61, 62, and 63 of the DMD gene. In some instances, the polynucleic acid molecule hybridizes to a target region that is about 5, 10, 15, 20, 50, 100, 200, 300, 400 or 500 bp downstream (or 3′) to at least one of exon 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 52, 53, 54, 55, 56, 57, 58, 59, 60, 61, 62, and 63 of the DMD gene. In some instances, the polynucleic acid molecule hybridizes to a target region that is about 5, 10, 15, 20, 50, 100, 200, 300, 400 or 500 bp downstream (or 3′) to at least one of exon 8, 23, 35, 43, 44, 45, 50, 51, 52, 53, or 55 of the DMD gene.

In some instances, a polynucleic acid molecule described herein targets an internal region within exon 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, or 63 of the DMD gene. In some instances, a polynucleic acid molecule described herein targets an internal region within exon 8, 23, 35, 43, 44, 45, 50, 51, 52, 53, or 55 of the DMD gene. In some instances, a polynucleic acid molecule described herein targets an internal region within exon 8 of the DMD gene. In some instances, a polynucleic acid molecule described herein targets an internal region within exon 23 of the DMD gene. In some instances, a polynucleic acid molecule described herein targets an internal region within exon 35 of the DMD gene. In some instances, a polynucleic acid molecule described herein targets an internal region within exon 43 of the DMD gene. In some instances, a polynucleic acid molecule described herein targets an internal region within exon 44 of the DMD gene. In some instances, a polynucleic acid molecule described herein targets an internal region within exon 45 of the DMD gene. In some instances, a polynucleic acid molecule described herein targets an internal region within exon 48 of the DMD gene. In some instances, a polynucleic acid molecule described herein targets an internal region within exon 49 of the DMD gene. In some instances, a polynucleic acid molecule described herein targets an internal region within exon 50 of the DMD gene. In some instances, a polynucleic acid molecule described herein targets an internal region within exon 51 of the DMD gene. In some instances, a polynucleic acid molecule described herein targets an internal region within exon 52 of the DMD gene. In some instances, a polynucleic acid molecule described herein targets an internal region within exon 53 of the DMD gene. In some instances, a polynucleic acid molecule described herein targets an internal region within exon 55 of the DMD gene.

In some instances, the polynucleic acid molecule hybridizes to a target region that is within at least one of exon 3, 4, 5, 6, 7, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 52, 53, 54, 55, 56, 57, 58, 59, 60, 61, 62, and 63 of the DMD gene. In some instances, the polynucleic acid molecule hybridizes to a target region that is within at least one of exon 8, 23, 35, 43, 44, 45, 50, 51, 52, 53, or 55 of the DMD gene.

In some aspects, a polynucleic acid molecule described herein targets a partially spliced mRNA sequence comprising exon 51. In some instances, the polynucleic acid molecule hybridizes to a target region that is upstream (or 5′) to exon 51. In some instances, the polynucleic acid molecule hybridizes to a target region that is about 5, 10, 15, 20, 50, 100, 200, 300, 400 or 500 bp upstream (or 5′) to exon 51. In some instances, the polynucleic acid molecule hybridizes to a target region that is downstream (or 3′) to exon 51. In some instances, the polynucleic acid molecule hybridizes to a target region that is about 5, 10, 15, 20, 50, 100, 200, 300, 400 or 500 bp downstream (or 3′) to exon 51.

In some instances, the polynucleic acid molecule hybridizes to a target region that is within exon 51. In some instances, the polynucleic acid molecule hybridizes to a target region that is at either the 5′ intron-exon 51 junction or the 3′ exon 51-intron junction.

In some aspects, the polynucleic acid molecule comprises a sequence having at least 50%, 55%, 60%, 65%, 70%, 75%, 80%, 85%, 90%, 95%, 96%, 97%, 98%, 99%, or 100% sequence identity to a target sequence of interest. In some aspects, the polynucleic acid molecule comprises a sequence having at least 50% sequence identity to a target sequence of interest. In some aspects, the polynucleic acid molecule comprises a sequence having at least 60% sequence identity to a target sequence of interest. In some aspects, the polynucleic acid molecule comprises a sequence having at least 70% sequence identity to a target sequence of interest. In some aspects, the polynucleic acid molecule comprises a sequence having at least 75% sequence identity to a target sequence of interest. In some aspects, the polynucleic acid molecule comprises a sequence having at least 80% sequence identity to a target sequence of interest. In some aspects, the polynucleic acid molecule comprises a sequence having at least 85% sequence identity to a target sequence of interest. In some aspects, the polynucleic acid molecule comprises a sequence having at least 90% sequence identity to a target sequence of interest. In some aspects, the polynucleic acid molecule comprises a sequence having at least 95% sequence identity to a target sequence of interest. In some aspects, the polynucleic acid molecule comprises a sequence having at least 96% sequence identity to a target sequence of interest. In some aspects, the polynucleic acid molecule comprises a sequence having at least 97% sequence identity to a target sequence of interest. In some aspects, the polynucleic acid molecule comprises a sequence having at least 98% sequence identity to a target sequence of interest. In some aspects, the polynucleic acid molecule comprises a sequence having at least 99% sequence identity to a target sequence of interest. In some aspects, the polynucleic acid molecule consists of a target sequence of interest.

In some aspects, the polynucleic acid molecule comprises a first polynucleotide and a second polynucleotide. In some instances, the first polynucleotide comprises a sequence having at least 50%, 55%, 60%, 65%, 70%, 75%, 80%, 85%, 90%, 95%, 96%, 97%, 98%, 99%, or 100% sequence identity to a target sequence of interest. In some cases, the second polynucleotide comprises a sequence having at least 50%, 55%, 60%, 65%, 70%, 75%, 80%, 85%, 90%, 95%, 96%, 97%, 98%, 99%, or 100% sequence identity to a target sequence of interest. In some cases, the polynucleic acid molecule comprises a first polynucleotide having at least 50%, 55%, 60%, 65%, 70%, 75%, 80%, 85%, 90%, 95%, 96%, 97%, 98%, 99%, or 100% sequence identity to a target sequence of interest and a second polynucleotide having at least 50%, 55%, 60%, 65%, 70%, 75%, 80%, 85%, 90%, 95%, 96%, 97%, 98%, 99%, or 100% sequence identity to a target sequence of interest.

In some aspects, the polynucleic acid molecule described herein comprises RNA or DNA.

In some cases, the polynucleic acid molecule comprises RNA. In some instances, RNA comprises short interfering RNA (siRNA), short hairpin RNA (shRNA), microRNA (miRNA), double-stranded RNA (dsRNA), transfer RNA (tRNA), ribosomal RNA (rRNA), or heterogeneous nuclear RNA (hnRNA). In some instances, RNA comprises shRNA. In some instances, RNA comprises miRNA. In some instances, RNA comprises dsRNA. In some instances, RNA comprises tRNA. In some instances, RNA comprises rRNA. In some instances, RNA comprises hnRNA. In some instances, the RNA comprises siRNA. In some instances, the polynucleic acid molecule comprises siRNA.

In some aspects, the polynucleic acid molecule is from about 10 to about 50 nucleotides in length. In some instances, the polynucleic acid molecule is from about 10 to about 30, from about 15 to about 30, from about 18 to about 25, form about 18 to about 24, from about 19 to about 23, or from about 20 to about 22 nucleotides in length.

In some aspects, the polynucleic acid molecule is about 50 nucleotides in length. In some instances, the polynucleic acid molecule is about 45 nucleotides in length. In some instances, the polynucleic acid molecule is about 40 nucleotides in length. In some instances, the polynucleic acid molecule is about 35 nucleotides in length. In some instances, the polynucleic acid molecule is about 30 nucleotides in length. In some instances, the polynucleic acid molecule is about 25 nucleotides in length. In some instances, the polynucleic acid molecule is about 20 nucleotides in length. In some instances, the polynucleic acid molecule is about 19 nucleotides in length. In some instances, the polynucleic acid molecule is about 18 nucleotides in length. In some instances, the polynucleic acid molecule is about 17 nucleotides in length. In some instances, the polynucleic acid molecule is about 16 nucleotides in length. In some instances, the polynucleic acid molecule is about 15 nucleotides in length. In some instances, the polynucleic acid molecule is about 14 nucleotides in length. In some instances, the polynucleic acid molecule is about 13 nucleotides in length. In some instances, the polynucleic acid molecule is about 12 nucleotides in length. In some instances, the polynucleic acid molecule is about 11 nucleotides in length. In some instances, the polynucleic acid molecule is about 10 nucleotides in length. In some instances, the polynucleic acid molecule is between about 10 and about 50 nucleotides in length.

In some instances, the polynucleic acid molecule is between about 10 and about 45 nucleotides in length. In some instances, the polynucleic acid molecule is between about 10 and about 40 nucleotides in length. In some instances, the polynucleic acid molecule is between about 10 and about 35 nucleotides in length. In some instances, the polynucleic acid molecule is between about 10 and about 30 nucleotides in length. In some instances, the polynucleic acid molecule is between about 10 and about 25 nucleotides in length. In some instances, the polynucleic acid molecule is between about 10 and about 20 nucleotides in length. In some instances, the polynucleic acid molecule is between about 15 and about 25 nucleotides in length. In some instances, the polynucleic acid molecule is between about 15 and about 30 nucleotides in length.

In some instances, the polynucleic acid molecule is between about 12 and about 30 nucleotides in length.

In some aspects, the polynucleic acid molecule comprises a first polynucleotide. In some instances, the polynucleic acid molecule comprises a second polynucleotide. In some instances, the polynucleic acid molecule comprises a first polynucleotide and a second polynucleotide. In some instances, the first polynucleotide is a sense strand or passenger strand. In some instances, the second polynucleotide is an antisense strand or guide strand.

In some aspects, the polynucleic acid molecule is a first polynucleotide. In some aspects, the first polynucleotide is from about 10 to about 50 nucleotides in length. In some instances, the first polynucleotide is from about 10 to about 30, from about 15 to about 30, from about 18 to about 25, form about 18 to about 24, from about 19 to about 23, or from about 20 to about 22 nucleotides in length.

In some instances, the first polynucleotide is about 50 nucleotides in length. In some instances, the first polynucleotide is about 45 nucleotides in length. In some instances, the first polynucleotide is about 40 nucleotides in length. In some instances, the first polynucleotide is about 35 nucleotides in length. In some instances, the first polynucleotide is about 30 nucleotides in length. In some instances, the first polynucleotide is about 25 nucleotides in length. In some instances, the first polynucleotide is about 20 nucleotides in length. In some instances, the first polynucleotide is about 19 nucleotides in length. In some instances, the first polynucleotide is about 18 nucleotides in length. In some instances, the first polynucleotide is about 17 nucleotides in length. In some instances, the first polynucleotide is about 16 nucleotides in length. In some instances, the first polynucleotide is about 15 nucleotides in length. In some instances, the first polynucleotide is about 14 nucleotides in length. In some instances, the first polynucleotide is about 13 nucleotides in length. In some instances, the first polynucleotide is about 12 nucleotides in length. In some instances, the first polynucleotide is about 11 nucleotides in length. In some instances, the first polynucleotide is about 10 nucleotides in length. In some instances, the first polynucleotide is between about 10 and about 50 nucleotides in length. In some instances, the first polynucleotide is between about 10 and about 45 nucleotides in length. In some instances, the first polynucleotide is between about 10 and about 40 nucleotides in length. In some instances, the first polynucleotide is between about 10 and about 35 nucleotides in length. In some instances, the first polynucleotide is between about 10 and about 30 nucleotides in length.

In some instances, the first polynucleotide is between about 10 and about 25 nucleotides in length. In some instances, the first polynucleotide is between about 10 and about 20 nucleotides in length. In some instances, the first polynucleotide is between about 15 and about 25 nucleotides in length. In some instances, the first polynucleotide is between about 15 and about 30 nucleotides in length. In some instances, the first polynucleotide is between about 12 and about 30 nucleotides in length.

In some aspects, the polynucleic acid molecule is a second polynucleotide. In some aspects, the second polynucleotide is from about 10 to about 50 nucleotides in length. In some instances, the second polynucleotide is from about 10 to about 30, from about 15 to about 30, from about 18 to about 25, form about 18 to about 24, from about 19 to about 23, or from about 20 to about 22 nucleotides in length.

In some instances, the second polynucleotide is about 50 nucleotides in length. In some instances, the second polynucleotide is about 45 nucleotides in length. In some instances, the second polynucleotide is about 40 nucleotides in length. In some instances, the second polynucleotide is about 35 nucleotides in length. In some instances, the second polynucleotide is about 30 nucleotides in length. In some instances, the second polynucleotide is about 25 nucleotides in length. In some instances, the second polynucleotide is about 20 nucleotides in length. In some instances, the second polynucleotide is about 19 nucleotides in length. In some instances, the second polynucleotide is about 18 nucleotides in length. In some instances, the second polynucleotide is about 17 nucleotides in length. In some instances, the second polynucleotide is about 16 nucleotides in length. In some instances, the second polynucleotide is about 15 nucleotides in length. In some instances, the second polynucleotide is about 14 nucleotides in length. In some instances, the second polynucleotide is about 13 nucleotides in length. In some instances, the second polynucleotide is about 12 nucleotides in length. In some instances, the second polynucleotide is about 11 nucleotides in length. In some instances, the second polynucleotide is about 10 nucleotides in length. In some instances, the second polynucleotide is between about 10 and about 50 nucleotides in length. In some instances, the second polynucleotide is between about 10 and about 45 nucleotides in length. In some instances, the second polynucleotide is between about 10 and about 40 nucleotides in length. In some instances, the second polynucleotide is between about 10 and about 35 nucleotides in length. In some instances, the second polynucleotide is between about 10 and about 30 nucleotides in length. In some instances, the second polynucleotide is between about 10 and about 25 nucleotides in length. In some instances, the second polynucleotide is between about 10 and about 20 nucleotides in length. In some instances, the second polynucleotide is between about 15 and about 25 nucleotides in length. In some instances, the second polynucleotide is between about 15 and about 30 nucleotides in length. In some instances, the second polynucleotide is between about 12 and about 30 nucleotides in length.

In some aspects, the polynucleic acid molecule comprises a first polynucleotide and a second polynucleotide. In some instances, the polynucleic acid molecule further comprises a blunt terminus, an overhang, or a combination thereof. In some instances, the blunt terminus is a 5′ blunt terminus, a 3′ blunt terminus, or both. In some cases, the overhang is a 5′ overhang, 3′ overhang, or both. In some cases, the overhang comprises 1, 2, 3, 4, 5, 6, 7, 8, 9, or 10 non-base pairing nucleotides. In some cases, the overhang comprises 1, 2, 3, 4, 5, or 6 non-base pairing nucleotides. In some cases, the overhang comprises 1, 2, 3, or 4 non-base pairing nucleotides. In some cases, the overhang comprises 1 non-base pairing nucleotide. In some cases, the overhang comprises 2 non-base pairing nucleotides. In some cases, the overhang comprises 3 non-base pairing nucleotides. In some cases, the overhang comprises 4 non-base pairing nucleotides.

In some aspects, the sequence of the polynucleic acid molecule is at least 40%, 50%, 55%, 60%, 65%, 70%, 75%, 80%, 85%, 90%, 95%, 98%, 99%, or 99.5% complementary to a target sequence described herein. In some aspects, the sequence of the polynucleic acid molecule is at least 50% complementary to a target sequence described herein. In some aspects, the sequence of the polynucleic acid molecule is at least 60% complementary to a target sequence described herein. In some aspects, the sequence of the polynucleic acid molecule is at least 70% complementary to a target sequence described herein. In some aspects, the sequence of the polynucleic acid molecule is at least 80% complementary to a target sequence described herein.

In some aspects, the sequence of the polynucleic acid molecule is at least 90% complementary to a target sequence described herein. In some aspects, the sequence of the polynucleic acid molecule is at least 95% complementary to a target sequence described herein. In some aspects, the sequence of the polynucleic acid molecule is at least 99% complementary to a target sequence described herein. In some instances, the sequence of the polynucleic acid molecule is 100% complementary to a target sequence described herein.

In some aspects, the sequence of the polynucleic acid molecule has 5 or less mismatches to a target sequence described herein. In some aspects, the sequence of the polynucleic acid molecule has 4 or less mismatches to a target sequence described herein. In some instances, the sequence of the polynucleic acid molecule has 3 or less mismatches to a target sequence described herein. In some cases, the sequence of the polynucleic acid molecule has 2 or less mismatches to a target sequence described herein. In some cases, the sequence of the polynucleic acid molecule has 1 or less mismatches to a target sequence described herein.

In some aspects, the specificity of the polynucleic acid molecule that hybridizes to a target sequence described herein is a 95%, 98%, 99%, 99.5% or 100% sequence complementarity of the polynucleic acid molecule to a target sequence. In some instances, the hybridization is a high stringent hybridization condition.

In some aspects, the polynucleic acid molecule has reduced off-target effect. In some instances, “off-target” or “off-target effects” refer to any instance in which a polynucleic acid polymer directed against a given target causes an unintended effect by interacting either directly or indirectly with another mRNA sequence, a DNA sequence or a cellular protein or other moiety. In some instances, an “off-target effect” occurs when there is a simultaneous degradation of other transcripts due to partial homology or complementarity between that other transcript and the sense and/or antisense strand of the polynucleic acid molecule.

In some aspects, the polynucleic acid molecule comprises natural or synthetic or artificial nucleotide analogues or bases. In some cases, the polynucleic acid molecule comprises combinations of DNA, RNA and/or nucleotide analogues. In some instances, the synthetic or artificial nucleotide analogues or bases comprise modifications at one or more of ribose moiety, phosphate moiety, nucleoside moiety, or a combination thereof.

In some aspects, nucleotide analogues or artificial nucleotide base comprise a nucleic acid with a modification at a 2′ hydroxyl group of the ribose moiety. In some instances, the modification includes an H, OR, R, halo, SH, SR, NH2, NHR, NR2, or CN, wherein R is an alkyl moiety. Exemplary alkyl moiety includes, but is not limited to, halogens, sulfurs, thiols, thioethers, thioesters, amines (primary, secondary, or tertiary), amides, ethers, esters, alcohols and oxygen. In some instances, the alkyl moiety further comprises a modification. In some instances, the modification comprises an azo group, a keto group, an aldehyde group, a carboxyl group, a nitro group, a nitroso, group, a nitrile group, a heterocycle (e.g., imidazole, hydrazino or hydroxylamino) group, an isocyanate or cyanate group, or a sulfur containing group (e.g., sulfoxide, sulfone, sulfide, and disulfide). In some instances, the alkyl moiety further comprises a hetero substitution. In some instances, the carbon of the heterocyclic group is substituted by a nitrogen, oxygen or sulfur. In some instances, the heterocyclic substitution includes but is not limited to, morpholino, imidazole, and pyrrolidino.

In some instances, the modification at the 2′ hydroxyl group is a 2′-O-methyl modification or a 2′-O-methoxyethyl (2′-O-MOE) modification. In some cases, the 2′-O-methyl modification adds a methyl group to the 2′ hydroxyl group of the ribose moiety whereas the 2′O-methoxyethyl modification adds a methoxyethyl group to the 2′ hydroxyl group of the ribose moiety. Exemplary chemical structures of a 2′-O-methyl modification of an adenosine molecule and 2′O-methoxyethyl modification of an uridine are illustrated below.

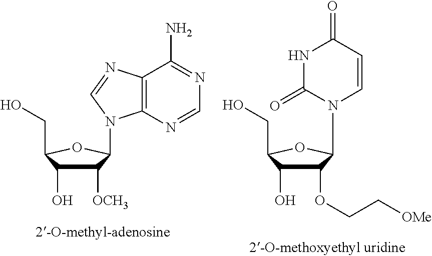

In some instances, the modification at the 2′ hydroxyl group is a 2′-O-aminopropyl modification in which an extended amine group comprising a propyl linker binds the amine group to the 2′ oxygen. In some instances, this modification neutralizes the phosphate derived overall negative charge of the oligonucleotide molecule by introducing one positive charge from the amine group per sugar and thereby improves cellular uptake properties due to its zwitterionic properties. An exemplary chemical structure of a 2′-O-aminopropyl nucleoside phosphoramidite is illustrated below.

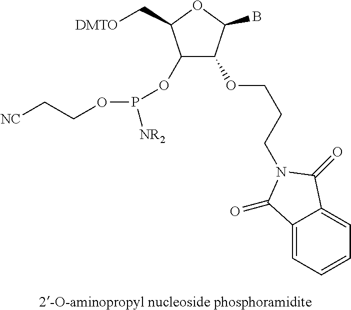

In some instances, the modification at the 2′ hydroxyl group is a locked or bridged ribose modification (e.g., locked nucleic acid or LNA) in which the oxygen molecule bound at the 2′ carbon is linked to the 4′ carbon by a methylene group, thus forming a 2′-C,4′-C-oxy-methylene-linked bicyclic ribonucleotide monomer. Exemplary representations of the chemical structure of LNA are illustrated below. The representation shown to the left highlights the chemical connectivities of an LNA monomer. The representation shown to the right highlights the locked 3′-endo (3E) conformation of the furanose ring of an LNA monomer.

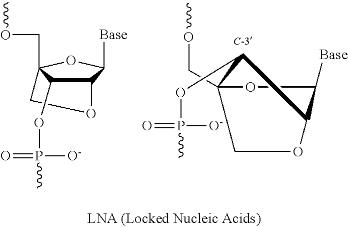

In some instances, the modification at the 2′ hydroxyl group comprises ethylene nucleic acids (ENA) such as for example 2′-4′-ethylene-bridged nucleic acid, which locks the sugar conformation into a C3′-endo sugar puckering conformation. ENA are part of the bridged nucleic acids class of modified nucleic acids that also comprises LNA. Exemplary chemical structures of the ENA and bridged nucleic acids are illustrated below.

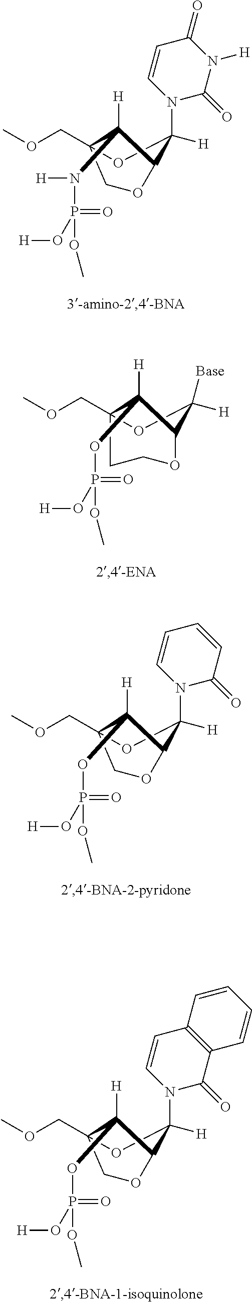

In some aspects, additional modifications at the 2′ hydroxyl group include 2′-deoxy, 2′-deoxy-2′-fluoro, 2′-O-aminopropyl (2′-O-AP), 2-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA).

In some aspects, nucleotide analogues comprise modified bases such as, but not limited to, 5-propynyluridine, 5-propynylcytidine, 6-methyladenine, 6-methylguanine, N, N, -dimethyladenine, 2-propyladenine, 2propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2, 2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4, 6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyi nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, in some cases are or be based on, mannoses, arabinoses, glucopyranoses, galactopyranoses, 4′-thioribose, and other sugars, heterocycles, or carbocycles. The term nucleotide also includes what are known in the art as universal bases. By way of example, universal bases include but are not limited to 3-nitropyrrole, 5-nitroindole, or nebularine.

In some aspects, nucleotide analogues further comprise morpholinos, peptide nucleic acids (PNAs), methylphosphonate nucleotides, thiolphosphonate nucleotides, 2′-fluoro N3-P5′-phosphoramidites, 1′, 5′-anhydrohexitol nucleic acids (HNAs), or a combination thereof. Morpholino or phosphorodiamidate morpholino oligo (PMO) comprises synthetic molecules whose structure mimics natural nucleic acid structure by deviates from the normal sugar and phosphate structures. In some instances, the five member ribose ring is substituted with a six member morpholino ring containing four carbons, one nitrogen and one oxygen. In some cases, the ribose monomers are linked by a phosphordiamidate group instead of a phosphate group. In such cases, the backbone alterations remove all positive and negative charges making morpholinos neutral molecules capable of crossing cellular membranes without the aid of cellular delivery agents such as those used by charged oligonucleotides.

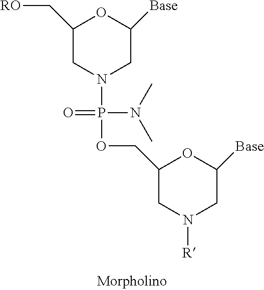

In some aspects, peptide nucleic acid (PNA) does not contain sugar ring or phosphate linkage and the bases are attached and appropriately spaced by oligoglycine-like molecules, therefore, eliminating a backbone charge.

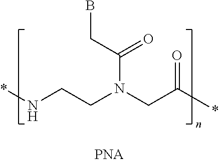

In some aspects, one or more modifications optionally occur at the intemucleotide linkage. In some instances, modified intemucleotide linkage include, but is not limited to, phosphorothioates, phosphorodithioates, methylphosphonates, 5′-alkylenephosphonates, 5′-methylphosphonate, 3′-alkylene phosphonates, borontrifluoridates, borano phosphate esters and selenophosphates of 3′-5′ linkage or 2′-5′ linkage, phosphotriesters, thionoalkylphosphotriesters, hydrogen phosphonate linkages, alkyl phosphonates, alkylphosphonothioates, arylphosphonothioates, phosphoroselenoates, phosphorodiselenoates, phosphinates, phosphoramidates, 3′-alkylphosphoramidates, aminoalkylphosphoramidates, thionophosphoramidates, phosphoropiperazidates, phosphoroanilothioates, phosphoroanilidates, ketones, sulfones, sulfonamides, carbonates, carbamates, methylenehydrazos, methylenedimethylhydrazos, formacetals, thioformacetals, oximes, methyleneiminos, methylenemethyliminos, thioamidates, linkages with riboacetyl groups, aminoethyl glycine, silyl or siloxane linkages, alkyl or cycloalkyl linkages with or without heteroatoms of, for example, 1 to 10 carbons that are saturated or unsaturated and/or substituted and/or contain heteroatoms, linkages with morpholino structures, amides, polyamides wherein the bases are attached to the aza nitrogens of the backbone directly or indirectly, and combinations thereof. Phosphorothioate antisene oligonucleotides (PS ASO) are antisense oligonucleotides comprising a phosphorothioate linkage. An exemplary PS ASO is illustrated below.

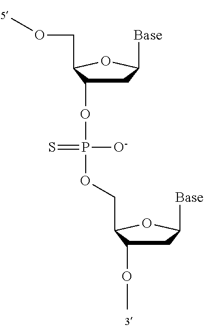

In some instances, the modification is a methyl or thiol modification such as methylphosphonate or thiolphosphonate modification. Exemplary thiolphosphonate nucleotide (left) and methylphosphonate nucleotide (right) are illustrated below.

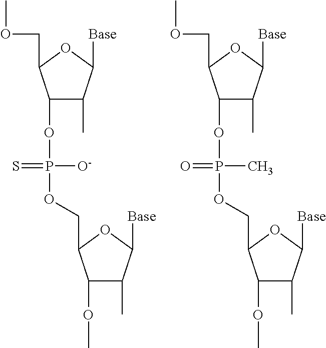

In some instances, a modified nucleotide includes, but is not limited to, 2′-fluoro N3-P5′-phosphoramidites illustrated as:

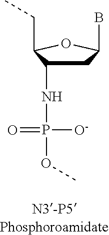

In some instances, a modified nucleotide includes, but is not limited to, hexitol nucleic acid (or 1′, 5′-anhydrohexitol nucleic acids (HNA)) illustrated as:

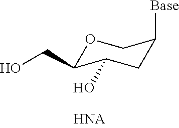

In some aspects, one or more modifications further optionally include modifications of the ribose moiety, phosphate backbone and the nucleoside, or modifications of the nucleotide analogues at the 3′ or the 5′ terminus. For example, the 3′ terminus optionally include a 3′ cationic group, or by inverting the nucleoside at the 3′-terminus with a 3′-3′ linkage. In another alternative, the 3′-terminus is optionally conjugated with an aminoalkyl group, e.g., a 3′ C5-aminoalkyl dT. In an additional alternative, the 3′-terminus is optionally conjugated with an abasic site, e.g., with an apurinic or apyrimidinic site. In some instances, the 5′-terminus is conjugated with an aminoalkyl group, e.g., a 5′-O-alkylamino substituent. In some cases, the 5′-terminus is conjugated with an abasic site, e.g., with an apurinic or apyrimidinic site.

In some aspects, the polynucleic acid molecule comprises one or more of the artificial nucleotide analogues described herein. In some instances, the polynucleic acid molecule comprises 1, 2, 3, 4, 5, 6, 7, 8, 9, 10, 11, 12, 13, 14, 15, 16, 17, 18, 20, 25, or more of the artificial nucleotide analogues described herein. In some aspects, the artificial nucleotide analogues include 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-0-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the polynucleic acid molecule comprises 1, 2, 3, 4, 5, 6, 7, 8, 9, 10, 11, 12, 13, 14, 15, 16, 17, 18, 20, 25, or more of the artificial nucleotide analogues selected from 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the polynucleic acid molecule comprises 1, 2, 3, 4, 5, 6, 7, 8, 9, 10, 11, 12, 13, 14, 15, 16, 17, 18, 20, 25, or more of 2′-O-methyl modified nucleotides. In some instances, the polynucleic acid molecule comprises 1, 2, 3, 4, 5, 6, 7, 8, 9, 10, 11, 12, 13, 14, 15, 16, 17, 18, 20, 25, or more of 2′-O-methoxyethyl (2′-O-MOE) modified nucleotides. In some instances, the polynucleic acid molecule comprises 1, 2, 3, 4, 5, 6, 7, 8, 9, 10, 11, 12, 13, 14, 15, 16, 17, 18, 20, 25, or more of thiolphosphonate nucleotides.

In some instances, the polynucleic acid molecule comprises at least one of: from about 5% to about 100% modification, from about 10% to about 100% modification, from about 20% to about 100% modification, from about 30% to about 100% modification, from about 40% to about 100% modification, from about 50% to about 100% modification, from about 60% to about 100% modification, from about 70% to about 100% modification, from about 80% to about 100% modification, and from about 90% to about 100% modification.

In some cases, the polynucleic acid molecule comprises at least one of: from about 10% to about 90% modification, from about 20% to about 90% modification, from about 30% to about 90% modification, from about 40% to about 90% modification, from about 50% to about 90% modification, from about 60% to about 90% modification, from about 70% to about 90% modification, and from about 80% to about 100% modification.

In some cases, the polynucleic acid molecule comprises at least one of: from about 10% to about 80% modification, from about 20% to about 80% modification, from about 30% to about 80% modification, from about 40% to about 80% modification, from about 50% to about 80% modification, from about 60% to about 80% modification, and from about 70% to about 80% modification.

In some instances, the polynucleic acid molecule comprises at least one of: from about 10% to about 70% modification, from about 20% to about 70% modification, from about 30% to about 70% modification, from about 40% to about 70% modification, from about 50% to about 70% modification, and from about 60% to about 70% modification.

In some instances, the polynucleic acid molecule comprises at least one of: from about 10% to about 60% modification, from about 20% to about 60% modification, from about 30% to about 60% modification, from about 40% to about 60% modification, and from about 50% to about 60% modification.

In some cases, the polynucleic acid molecule comprises at least one of: from about 10% to about 50% modification, from about 20% to about 50% modification, from about 30% to about 50% modification, and from about 40% to about 50% modification.

In some cases, the polynucleic acid molecule comprises at least one of: from about 10% to about 40% modification, from about 20% to about 40% modification, and from about 30% to about 40% modification.

In some cases, the polynucleic acid molecule comprises at least one of: from about 10% to about 30% modification, and from about 20% to about 30% modification.

In some cases, the polynucleic acid molecule comprises from about 10% to about 20% modification.

In some cases, the polynucleic acid molecule comprises from about 15% to about 90%, from about 20% to about 80%, from about 30% to about 70%, or from about 40% to about 60% modifications.

In additional cases, the polynucleic acid molecule comprises at least about 15%, 20%, 30%, 40%, 50%, 60%, 70%, 80%, 90%, 95%, or 99% modification.

In some aspects, the polynucleic acid molecule comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22 or more modifications.

In some instances, the polynucleic acid molecule comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22 or more modified nucleotides.

In some instances, from about 5% to about 100% of the polynucleic acid molecule comprise the artificial nucleotide analogues described herein. In some instances, about 5%, 10%, 15%, 20%, 25%, 30%, 35%, 40%, 45%, 50%, 55%, 60%, 65%, 70%, 75%, 80%, 85%, 90%, 95% or 100% of the polynucleic acid molecule comprise the artificial nucleotide analogues described herein. In some instances, about 5% of a polynucleic acid molecule comprise the artificial nucleotide analogues described herein. In some instances, about 10% of a polynucleic acid molecule comprise the artificial nucleotide analogues described herein. In some instances, about 15% of a polynucleic acid molecule comprise the artificial nucleotide analogues described herein. In some instances, about 20% of a polynucleic acid molecule comprise the artificial nucleotide analogues described herein. In some instances, about 25% of a polynucleic acid molecule comprise the artificial nucleotide analogues described herein. In some instances, about 30% of a polynucleic acid molecule comprise the artificial nucleotide analogues described herein. In some instances, about 35% of a polynucleic acid molecule comprise the artificial nucleotide analogues described herein. In some instances, about 40% of a polynucleic acid molecule comprise the artificial nucleotide analogues described herein. In some instances, about 45% of a polynucleic acid molecule comprise the artificial nucleotide analogues described herein. In some instances, about 50% of a polynucleic acid molecule comprise the artificial nucleotide analogues described herein. In some instances, about 55% of a polynucleic acid molecule comprise the artificial nucleotide analogues described herein. In some instances, about 60% of a polynucleic acid molecule comprise the artificial nucleotide analogues described herein. In some instances, about 65% of a polynucleic acid molecule comprise the artificial nucleotide analogues described herein. In some instances, about 70% of a polynucleic acid molecule comprise the artificial nucleotide analogues described herein. In some instances, about 75% of a polynucleic acid molecule comprise the artificial nucleotide analogues described herein. In some instances, about 80% of a polynucleic acid molecule comprise the artificial nucleotide analogues described herein. In some instances, about 85% of a polynucleic acid molecule comprise the artificial nucleotide analogues described herein. In some instances, about 90% of a polynucleic acid molecule comprise the artificial nucleotide analogues described herein. In some instances, about 95% of a polynucleic acid molecule comprise the artificial nucleotide analogues described herein. In some instances, about 96% of a polynucleic acid molecule comprise the artificial nucleotide analogues described herein. In some instances, about 97% of a polynucleic acid molecule comprise the artificial nucleotide analogues described herein. In some instances, about 98% of a polynucleic acid molecule comprise the artificial nucleotide analogues described herein. In some instances, about 99% of a polynucleic acid molecule comprise the artificial nucleotide analogues described herein. In some instances, about 100% of a polynucleic acid molecule comprise the artificial nucleotide analogues described herein. In some aspects, the artificial nucleotide analogues include 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof.

In some aspects, the polynucleic acid molecule comprises from about 1 to about 25 modifications in which the modification comprises an artificial nucleotide analogues described herein. In some aspects, a polynucleic acid molecule comprises about 1 modification in which the modification comprises an artificial nucleotide analogue described herein. In some aspects, a polynucleic acid molecule comprises about 2 modifications in which the modifications comprise an artificial nucleotide analogue described herein. In some aspects, a polynucleic acid molecule comprises about 3 modifications in which the modifications comprise an artificial nucleotide analogue described herein. In some aspects, a polynucleic acid molecule comprises about 4 modifications in which the modifications comprise an artificial nucleotide analogue described herein. In some aspects, a polynucleic acid molecule comprises about 5 modifications in which the modifications comprise an artificial nucleotide analogue described herein. In some aspects, a polynucleic acid molecule comprises about 6 modifications in which the modifications comprise an artificial nucleotide analogue described herein. In some aspects, a polynucleic acid molecule comprises about 7 modifications in which the modifications comprise an artificial nucleotide analogue described herein. In some aspects, a polynucleic acid molecule comprises about 8 modifications in which the modifications comprise an artificial nucleotide analogue described herein. In some aspects, a polynucleic acid molecule comprises about 9 modifications in which the modifications comprise an artificial nucleotide analogue described herein. In some aspects, a polynucleic acid molecule comprises about 10 modifications in which the modifications comprise an artificial nucleotide analogue described herein. In some aspects, a polynucleic acid molecule comprises about 11 modifications in which the modifications comprise an artificial nucleotide analogue described herein. In some aspects, a polynucleic acid molecule comprises about 12 modifications in which the modifications comprise an artificial nucleotide analogue described herein. In some aspects, a polynucleic acid molecule comprises about 13 modifications in which the modifications comprise an artificial nucleotide analogue described herein. In some aspects, a polynucleic acid molecule comprises about 14 modifications in which the modifications comprise an artificial nucleotide analogue described herein. In some aspects, a polynucleic acid molecule comprises about 15 modifications in which the modifications comprise an artificial nucleotide analogue described herein. In some aspects, a polynucleic acid molecule comprises about 16 modifications in which the modifications comprise an artificial nucleotide analogue described herein. In some aspects, a polynucleic acid molecule comprises about 17 modifications in which the modifications comprise an artificial nucleotide analogue described herein. In some aspects, a polynucleic acid molecule comprises about 18 modifications in which the modifications comprise an artificial nucleotide analogue described herein. In some aspects, a polynucleic acid molecule comprises about 19 modifications in which the modifications comprise an artificial nucleotide analogue described herein. In some aspects, a polynucleic acid molecule comprises about 20 modifications in which the modifications comprise an artificial nucleotide analogue described herein. In some aspects, a polynucleic acid molecule comprises about 21 modifications in which the modifications comprise an artificial nucleotide analogue described herein. In some aspects, a polynucleic acid molecule comprises about 22 modifications in which the modifications comprise an artificial nucleotide analogue described herein. In some aspects, a polynucleic acid molecule comprises about 23 modifications in which the modifications comprise an artificial nucleotide analogue described herein. In some aspects, a polynucleic acid molecule comprises about 24 modifications in which the modifications comprise an artificial nucleotide analogue described herein. In some aspects, a polynucleic acid molecule comprises about 25 modifications in which the modifications comprise an artificial nucleotide analogue described herein.

In some aspects, a polynucleic acid molecule is assembled from two separate polynucleotides wherein one polynucleotide comprises the sense strand and the second polynucleotide comprises the antisense strand of the polynucleic acid molecule. In other aspects, the sense strand is connected to the antisense strand via a linker molecule, which in some instances is a polynucleotide linker or a non-nucleotide linker.

In some aspects, a polynucleic acid molecule comprises a sense strand and antisense strand, wherein pyrimidine nucleotides in the sense strand comprises 2′-O-methylpyrimidine nucleotides and purine nucleotides in the sense strand comprise 2′-deoxy purine nucleotides. In some aspects, a polynucleic acid molecule comprises a sense strand and antisense strand, wherein pyrimidine nucleotides present in the sense strand comprise 2′-deoxy-2′-fluoro pyrimidine nucleotides and wherein purine nucleotides present in the sense strand comprise 2′-deoxy purine nucleotides.

In some aspects, a polynucleic acid molecule comprises a sense strand and antisense strand, wherein the pyrimidine nucleotides when present in said antisense strand are 2′-deoxy-2′-fluoro pyrimidine nucleotides and the purine nucleotides when present in said antisense strand are 2′-O-methyl purine nucleotides.

In some aspects, a polynucleic acid molecule comprises a sense strand and antisense strand, wherein the pyrimidine nucleotides when present in said antisense strand are 2′-deoxy-2′-fluoro pyrimidine nucleotides and wherein the purine nucleotides when present in said antisense strand comprise 2′-deoxy-purine nucleotides.

In some aspects, a polynucleic acid molecule comprises a sense strand and antisense strand, wherein the sense strand includes a terminal cap moiety at the 5′-end, the 3′-end, or both of the 5′ and 3′ ends of the sense strand. In other aspects, the terminal cap moiety is an inverted deoxy abasic moiety.

In some aspects, a polynucleic acid molecule comprises a sense strand and an antisense strand, wherein the antisense strand comprises a phosphate backbone modification at the 3′ end of the antisense strand. In some instances, the phosphate backbone modification is a phosphorothioate.

In some aspects, a polynucleic acid molecule comprises a sense strand and an antisense strand, wherein the antisense strand comprises a glyceryl modification at the 3′ end of the antisense strand.

In some aspects, a polynucleic acid molecule comprises a sense strand and an antisense strand, in which the sense strand comprises one or more, for example, about 1, 2, 3, 4, 5, 6, 7, 8, 9, 10, 11, 12, 13, 14, 15, 16, 17, 18, 19, 20, or more phosphorothioate intemucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or about one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the sense strand; and in which the antisense strand comprises about 1 to about 10 or more, specifically about 1, 2, 3, 4, 5, 6, 7, 8, 9, 10, 11, 12, 13, 14, 15, 16, 17, 18, 19, 20, or more phosphorothioate intemucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In other aspects, one or more, for example about 1, 2, 3, 4, 5, 6, 7, 8, 9, 10, or more, pyrimidine nucleotides of the sense and/or antisense strand are chemically-modified with 2′-deoxy, 2′-O-methyl and/or 2′-deoxy-2′-fluoro nucleotides, with or without one or more, for example about 1, 2, 3, 4, 5, 6, 7, 8, 9, 10, or more, phosphorothioate intemucleotide linkages and/or a terminal cap molecule at the 3′-end, the 5′-end, or both of the 3′- and 5′-ends, being present in the same or different strand.

In some aspects, a polynucleic acid molecule comprises a sense strand and an antisense strand, in which the sense strand comprises about 1 to about 25, for example, about 1, 2, 3, 4, 5, 6, 7, 8, 9, 10, 11, 12, 13, 14, 15, 16, 17, 18, 19, 20, or more phosphorothioate intemucleotide linkages, and/or one or more (e.g., about 1, 2, 3, 4, 5, 6, 7, 8, 9, 10, or more) 2′-deoxy, 2′-O-methyl, 2′-deoxy-2′-fluoro, and/or one or more (e.g., about 1, 2, 3, 4, 5, 6, 7, 8, 9, 10, or more) universal base modified nucleotides, and optionally a terminal cap molecule at the 3-end, the 5′-end, or both of the 3′- and 5′-ends of the sense strand; and in which the antisense strand comprises about 1 to about 25 or more, for example about 1, 2, 3, 4, 5, 6, 7, 8, 9, 10, 11, 12, 13, 14, 15, 16, 17, 18, 19, 20, or more phosphorothioate intemucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In other aspects, one or more, for example about 1, 2, 3, 4, 5, 6, 7, 8, 9, 10, or more, pyrimidine nucleotides of the sense and/or antisense strand are chemically-modified with 2′-deoxy, 2′-O-methyl and/or 2′-deoxy-2′-fluoro nucleotides, with or without about 1 to about 25 or more, for example about 1, 2, 3, 4, 5, 6, 7, 8, 9, 10, 11, 12, 13, 14, 15, 16, 17, 18, 19, 20, or more phosphorothioate intemucleotide linkages and/or a terminal cap molecule at the 3′-end, the 5′-end, or both of the 3′- and 5′-ends, being present in the same or different strand.

In some aspects, a polynucleic acid molecule comprises a sense strand and an antisense strand, in which the antisense strand comprises one or more, for example, about 1, 2, 3, 4, 5, 6, 7, 8, 9, 10, 11, 12, 13, 14, 15, 16, 17, 18, 19, 20, or more phosphorothioate intemucleotide linkages, and/or about one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the sense strand; and wherein the antisense strand comprises about 1 to about 10 or more, specifically about 1, 2, 3, 4, 5, 6, 7, 8, 9, 10 or more phosphorothioate intemucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In other aspects, one or more, for example about 1, 2, 3, 4, 5, 6, 7, 8, 9, 10, 11, 12, 13, 14, 15, 16, 17, 18, 19, 20, or more pyrimidine nucleotides of the sense and/or antisense strand are chemically-modified with 2′-deoxy, 2′-O-methyl and/or 2′-deoxy-2′-fluoro nucleotides, with or without one or more, for example, about 1, 2, 3, 4, 5, 6, 7, 8, 9, 10 or more phosphorothioate intemucleotide linkages and/or a terminal cap molecule at the 3′-end, the 5′-end, or both of the 3′ and 5′-ends, being present in the same or different strand.

In some aspects, a polynucleic acid molecule comprises a sense strand and an antisense strand, in which the antisense strand comprises about 1 to about 25 or more, for example, about 1, 2, 3, 4, 5, 6, 7, 8, 9, 10, 11, 12, 13, 14, 15, 16, 17, 18, 19, 20, or more phosphorothioate intemucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the sense strand; and wherein the antisense strand comprises about 1 to about 25 or more, for example about 1, 2, 3, 4, 5, 6, 7, 8, 9, 10, 11, 12, 13, 14, 15, 16, 17, 18, 19, 20, or more phosphorothioate intemucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In other aspects, one or more, for example about 1, 2, 3, 4, 5, 6, 7, 8, 9, 10 or more pyrimidine nucleotides of the sense and/or antisense strand are chemically-modified with 2′-deoxy, 2′-O-methyl and/or 2′-deoxy-2′-fluoro nucleotides, with or without about 1 to about 5, for example about 1, 2, 3, 4, 5 or more phosphorothioate intemucleotide linkages and/or a terminal cap molecule at the 3′-end, the 5′-end, or both of the 3′- and 5′-ends, being present in the same or different strand.

In some aspects, a polynucleic acid molecule described herein is a chemically-modified short interfering nucleic acid molecule having about 1 to about 25, for example, about 1, 2, 3, 4, 5, 6, 7, 8, 9, 10, 11, 12, 13, 14, 15, 16, 17, 18, 19, 20 or more phosphorothioate internucleotide linkages in each strand of the polynucleic acid molecule.

In another embodiment, a polynucleic acid molecule described herein comprises 2′-5′ intemucleotide linkages. In some instances, the 2′-5′ internucleotide linkage(s) is at the 3′-end, the 5′-end, or both of the 3′- and 5′-ends of one or both sequence strands. In addition instances, the 2′-5′ intemucleotide linkage(s) is present at various other positions within one or both sequence strands, for example, about 1, 2, 3, 4, 5, 6, 7, 8, 9, 10, or more including every intemucleotide linkage of a pyrimidine nucleotide in one or both strands of the polynucleic acid molecule comprise a 2′-5′ internucleotide linkage, or about 1, 2, 3, 4, 5, 6, 7, 8, 9, 10, or more including every intemucleotide linkage of a purine nucleotide in one or both strands of the polynucleic acid molecule comprise a 2′-5′ internucleotide linkage.

In some aspects, a polynucleic acid molecule is a single stranded polynucleic acid molecule that mediates RNAi activity in a cell or reconstituted in vitro system, wherein the polynucleic acid molecule comprises a single stranded polynucleotide having complementarity to a target nucleic acid sequence, and wherein one or more pyrimidine nucleotides present in the polynucleic acid are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any purine nucleotides present in the polynucleic acid are 2′-deoxy purine nucleotides (e.g., wherein all purine nucleotides are 2′-deoxy purine nucleotides or alternately a plurality of purine nucleotides are 2′-deoxy purine nucleotides), and a terminal cap modification, that is optionally present at the 3′-end, the 5′-end, or both of the 3′ and 5′-ends of the antisense sequence, the polynucleic acid molecule optionally further comprising about 1 to about 4 (e.g., about 1, 2, 3, or 4) terminal 2′-deoxynucleotides at the 3′-end of the polynucleic acid molecule, wherein the terminal nucleotides further comprise one or more (e.g., 1, 2, 3, or 4) phosphorothioate intemucleotide linkages, and wherein the polynucleic acid molecule optionally further comprises a terminal phosphate group, such as a 5′-terminal phosphate group.

In some cases, one or more of the artificial nucleotide analogues described herein are resistant toward nucleases such as for example ribonuclease such as RNase H, deoxyribonuclease such as DNase, or exonuclease such as 5′-3′ exonuclease and 3′-5′ exonuclease when compared to natural polynucleic acid molecules. In some instances, artificial nucleotide analogues comprising 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or combinations thereof are resistant toward nucleases such as for example ribonuclease such as RNase H, deoxyribonuclease such as DNase, or exonuclease such as 5′-3′ exonuclease and 3′-5′ exonuclease. In some instances, 2′-O-methyl modified polynucleic acid molecule is nuclease resistance (e.g., RNase H, DNase, 5′-3′ exonuclease or 3′-5′ exonuclease resistance). In some instances, 2′O-methoxyethyl (2′-O-MOE) modified polynucleic acid molecule is nuclease resistance (e.g., RNase H, DNase, 5′-3′ exonuclease or 3′-5′ exonuclease resistance). In some instances, 2′-O-aminopropyl modified polynucleic acid molecule is nuclease resistance (e.g., RNase H, DNase, 5′-3′ exonuclease or 3′-5′ exonuclease resistance). In some instances, 2′-deoxy modified polynucleic acid molecule is nuclease resistance (e.g., RNase H, DNase, 5′-3′ exonuclease or 3′-5′ exonuclease resistance). In some instances, 2′-deoxy-2′-fluoro modified polynucleic acid molecule is nuclease resistance (e.g., RNase H, DNase, 5′-3′ exonuclease or 3′-5′ exonuclease resistance). In some instances, 2′-O-aminopropyl (2′-O-AP) modified polynucleic acid molecule is nuclease resistance (e.g., RNase H, DNase, 5′-3′ exonuclease or 3′-5′ exonuclease resistance). In some instances, 2′-O-dimethylaminoethyl (2′-O-DMAOE) modified polynucleic acid molecule is nuclease resistance (e.g., RNase H, DNase, 5′-3′ exonuclease or 3′-5′ exonuclease resistance). In some instances, 2′-O-dimethylaminopropyl (2′-O-DMAP) modified polynucleic acid molecule is nuclease resistance (e.g., RNase H, DNase, 5′-3′ exonuclease or 3′-5′ exonuclease resistance). In some instances, 2′-O-dimethylaminoethyloxyethyl (2′-O-DMAEOE) modified polynucleic acid molecule is nuclease resistance (e.g., RNase H, DNase, 5′-3′ exonuclease or 3′-5′ exonuclease resistance). In some instances, 2′-O—N-methylacetamido (2′-O-NMA) modified polynucleic acid molecule is nuclease resistance (e.g., RNase H, DNase, 5′-3′ exonuclease or 3′-5′ exonuclease resistance). In some instances, LNA modified polynucleic acid molecule is nuclease resistance (e.g., RNase H, DNase, 5′-3′ exonuclease or 3′-5′ exonuclease resistance). In some instances, ENA modified polynucleic acid molecule is nuclease resistance (e.g., RNase H, DNase, 5′-3′ exonuclease or 3′-5′ exonuclease resistance). In some instances, HNA modified polynucleic acid molecule is nuclease resistance (e.g., RNase H, DNase, 5′-3′ exonuclease or 3′-5′ exonuclease resistance). In some instances, morpholinos is nuclease resistance (e.g., RNase H, DNase, 5′-3′ exonuclease or 3′-5′ exonuclease resistance). In some instances, PNA modified polynucleic acid molecule is resistant to nucleases (e.g., RNase H, DNase, 5′-3′ exonuclease or 3′-5′ exonuclease resistance).

In some instances, methylphosphonate nucleotides modified polynucleic acid molecule is nuclease resistance (e.g., RNase H, DNase, 5′-3′ exonuclease or 3′-5′ exonuclease resistance). In some instances, thiolphosphonate nucleotides modified polynucleic acid molecule is nuclease resistance (e.g., RNase H, DNase, 5′-3′ exonuclease or 3′-5′ exonuclease resistance). In some instances, polynucleic acid molecule comprising 2′-fluoro N3-P5′-phosphoramidites is nuclease resistance (e.g., RNase H, DNase, 5′-3′ exonuclease or 3′-5′ exonuclease resistance). In some instances, the 5′ conjugates described herein inhibit 5′-3′ exonucleolytic cleavage. In some instances, the 3′ conjugates described herein inhibit 3′-5′ exonucleolytic cleavage.

In some aspects, one or more of the artificial nucleotide analogues described herein have increased binding affinity toward their mRNA target relative to an equivalent natural polynucleic acid molecule. The one or more of the artificial nucleotide analogues comprising 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2-O-dimethylaminoethyl (2′-O-DMAOE), 2-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, or 2′-fluoro N3-P5′-phosphoramidites have increased binding affinity toward their mRNA target relative to an equivalent natural polynucleic acid molecule. In some instances, 2′-O-methyl modified polynucleic acid molecule has increased binding affinity toward their mRNA target relative to an equivalent natural polynucleic acid molecule. In some instances, 2′-O-methoxyethyl (2′-O-MOE) modified polynucleic acid molecule has increased binding affinity toward their mRNA target relative to an equivalent natural polynucleic acid molecule. In some instances, 2′-O-aminopropyl modified polynucleic acid molecule has increased binding affinity toward their mRNA target relative to an equivalent natural polynucleic acid molecule. In some instances, 2′-deoxy modified polynucleic acid molecule has increased binding affinity toward their mRNA target relative to an equivalent natural polynucleic acid molecule. In some instances, 2′-deoxy-2′-fluoro modified polynucleic acid molecule has increased binding affinity toward their mRNA target relative to an equivalent natural polynucleic acid molecule. In some instances, 2-O-aminopropyl (2′-O-AP) modified polynucleic acid molecule has increased binding affinity toward their mRNA target relative to an equivalent natural polynucleic acid molecule. In some instances, 2′-O-dimethylaminoethyl (2′-O-DMAOE) modified polynucleic acid molecule has increased binding affinity toward their mRNA target relative to an equivalent natural polynucleic acid molecule. In some instances, 2′-O-dimethylaminopropyl (2′-O-DMAP) modified polynucleic acid molecule has increased binding affinity toward their mRNA target relative to an equivalent natural polynucleic acid molecule. In some instances, 2′-O-dimethylaminoethyloxyethyl (2′-O-DMAEOE) modified polynucleic acid molecule has increased binding affinity toward their mRNA target relative to an equivalent natural polynucleic acid molecule. In some instances, 2′-O—N-methylacetamido (2′-O-NMA) modified polynucleic acid molecule has increased binding affinity toward their mRNA target relative to an equivalent natural polynucleic acid molecule. In some instances, LNA modified polynucleic acid molecule has increased binding affinity toward their mRNA target relative to an equivalent natural polynucleic acid molecule. In some instances, ENA modified polynucleic acid molecule has increased binding affinity toward their mRNA target relative to an equivalent natural polynucleic acid molecule. In some instances, PNA modified polynucleic acid molecule has increased binding affinity toward their mRNA target relative to an equivalent natural polynucleic acid molecule. In some instances, HNA modified polynucleic acid molecule has increased binding affinity toward their mRNA target relative to an equivalent natural polynucleic acid molecule. In some instances, morpholino modified polynucleic acid molecule has increased binding affinity toward their mRNA target relative to an equivalent natural polynucleic acid molecule. In some instances, methylphosphonate nucleotides modified polynucleic acid molecule has increased binding affinity toward their mRNA target relative to an equivalent natural polynucleic acid molecule. In some instances, thiolphosphonate nucleotides modified polynucleic acid molecule has increased binding affinity toward their mRNA target relative to an equivalent natural polynucleic acid molecule. In some instances, polynucleic acid molecule comprising 2′-fluoro N3-P5′-phosphoramidites has increased binding affinity toward their mRNA target relative to an equivalent natural polynucleic acid molecule. In some cases, the increased affinity is illustrated with a lower Kd, a higher melt temperature (Tm), or a combination thereof.

In some aspects, a polynucleic acid molecule described herein is a chirally pure (or stereo pure) polynucleic acid molecule, or a polynucleic acid molecule comprising a single enantiomer. In some instances, the polynucleic acid molecule comprises L-nucleotide. In some instances, the polynucleic acid molecule comprises D-nucleotides. In some instance, a polynucleic acid molecule composition comprises less than 30%, 25%, 20%, 15%, 10%, 5%, 4%, 3%, 2%, 1%, or less of its mirror enantiomer. In some cases, a polynucleic acid molecule composition comprises less than 30%, 25%, 20%, 15%, 10%, 5%, 4%, 3%, 2%, 1%, or less of a racemic mixture. In some instances, the polynucleic acid molecule is a polynucleic acid molecule described in: U.S. Patent Publication Nos: 2014/194610 and 2015/211006; and PCT Publication No.: WO2015107425.

In some aspects, a polynucleic acid molecule described herein is further modified to include an aptamer conjugating moiety. In some instances, the aptamer conjugating moiety is a DNA aptamer conjugating moiety. In some instances, the aptamer conjugating moiety is Alphamer (Centauri Therapeutics), which comprises an aptamer portion that recognizes a specific cell-surface target and a portion that presents a specific epitopes for attaching to circulating antibodies. In some instance, a polynucleic acid molecule described herein is further modified to include an aptamer conjugating moiety as described in: U.S. Pat. Nos. 8,604,184, 8,591,910, and 7,850,975.

In additional aspects, a polynucleic acid molecule described herein is modified to increase its stability. In some embodiment, the polynucleic acid molecule is RNA (e.g., siRNA). In some instances, the polynucleic acid molecule is modified by one or more of the modifications described above to increase its stability. In some cases, the polynucleic acid molecule is modified at the 2′ hydroxyl position, such as by 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2-O-dimethylaminopropyl (2′-O-DMAP), 2′-O-dimethylaminoethyloxyethyl (2′-O-DMAEOE), or 2′-O—N-methylacetamido (2′-O-NMA) modification or by a locked or bridged ribose conformation (e.g., LNA or ENA). In some cases, the polynucleic acid molecule is modified by 2′-O-methyl and/or 2′-O-methoxyethyl ribose. In some cases, the polynucleic acid molecule also includes morpholinos, PNAs, HNA, methylphosphonate nucleotides, thiolphosphonate nucleotides, and/or 2′-fluoro N3-P5′-phosphoramidites to increase its stability. In some instances, the polynucleic acid molecule is a chirally pure (or stereo pure) polynucleic acid molecule. In some instances, the chirally pure (or stereo pure) polynucleic acid molecule is modified to increase its stability. Suitable modifications to the RNA to increase stability for delivery will be apparent to the skilled person.

In some aspects, a polynucleic acid molecule describe herein has RNAi activity that modulates expression of RNA encoded by a gene involved in a disease or disorder such as, but not limited to, IKBKAP, SMN2, MCAD, LMNA, DMPK, ZNF9, MAPT, FKTN, TDP-43, LDLR, CFTR, DMD, PAH, MSTN, or K-Ras. In some instances, a polynucleic acid molecule describe herein is a double-stranded siRNA molecule that down-regulates expression of at least one of IKBKAP, SMN2, MCAD, LMNA, DMPK, ZNF9, MAPT, FKTN, TDP-43, LDLR, CFTR, DMD, PAH, MSTN, or K-Ras, wherein one of the strands of the double-stranded siRNA molecule comprises a nucleotide sequence that is complementary to a nucleotide sequence of at least one of IKBKAP, SMN2, MCAD, LMNA, DMPK, ZNF9, MAPT, FKTN, TDP-43, LDLR, CFTR, DMD, PAH, MSTN, or K-Ras or RNA encoded by at least one of IKBKAP, SMN2, MCAD, LMNA, DMPK, ZNF9, MAPT, FKTN, TDP-43, LDLR, CFTR, DMD, PAH, MSTN, or K-Ras or a portion thereof, and wherein the second strand of the double-stranded siRNA molecule comprises a nucleotide sequence substantially similar to the nucleotide sequence of at least one of IKBKAP, SMN2, MCAD, LMNA, DMPK, ZNF9, MAPT, FKTN, TDP-43, LDLR, CFTR, DMD, PAH, MSTN, or K-Ras or RNA encoded by at least one of IKBKAP, SMN2, MCAD, LMNA, DMPK, ZNF9, MAPT, FKTN, TDP-43, LDLR, CFTR, DMD, PAH, MSTN, or K-Ras or a portion thereof. In some cases, a polynucleic acid molecule describe herein is a double-stranded siRNA molecule that down-regulates expression of at least one of IKBKAP, SMN2, MCAD, LMNA, DMPK, ZNF9, MAPT, FKTN, TDP-43, LDLR, CFTR, DMD, PAH, MSTN, or K-Ras, wherein each strand of the siRNA molecule comprises about 15 to 25, 18 to 24, or 19 to about 23 nucleotides, and wherein each strand comprises at least about 14, 17, or 19 nucleotides that are complementary to the nucleotides of the other strand. In some cases, a polynucleic acid molecule describe herein is a double-stranded siRNA molecule that down-regulates expression of at least one of IKBKAP, SMN2, MCAD, LMNA, DMPK, ZNF9, MAPT, FKTN, TDP-43, LDLR, CFTR, DMD, PAH, MSTN, or K-Ras, wherein each strand of the siRNA molecule comprises about 19 to about 23 nucleotides, and wherein each strand comprises at least about 19 nucleotides that are complementary to the nucleotides of the other strand. In some instances, the RNAi activity occurs within a cell. In other instances, the RNAi activity occurs in a reconstituted in vitro system.

In some aspects, a polynucleic acid molecule describe herein has RNAi activity that modulates expression of RNA encoded by a gene involved in muscular dystrophy such as, but not limited to, DMD, DUX4, DYSF, EMD, or LMNA. In some instances, a polynucleic acid molecule describe herein is a double-stranded siRNA molecule that down-regulates expression of at least one of DMD, DUX4, DYSF, EMD, or LMNA, wherein one of the strands of the double-stranded siRNA molecule comprises a nucleotide sequence that is complementary to a nucleotide sequence of at least one of DMD, DUX4, DYSF, EMD, or LMNA or RNA encoded by at least one of DMD, DUX4, DYSF, EMD, or LMNA or a portion thereof, and wherein the second strand of the double-stranded siRNA molecule comprises a nucleotide sequence substantially similar to the nucleotide sequence of at least one of DMD, DUX4, DYSF, EMD, or LMNA or RNA encoded by at least one of DMD, DUX4, DYSF, EMD, or LMNA or a portion thereof. In some cases, a polynucleic acid molecule describe herein is a double-stranded siRNA molecule that down-regulates expression of at least one of DMD, DUX4, DYSF, EMD, or LMNA, wherein each strand of the siRNA molecule comprises about 15 to 25, 18 to 24, or 19 to about 23 nucleotides, and wherein each strand comprises at least about 14, 17, or 19 nucleotides that are complementary to the nucleotides of the other strand. In some cases, a polynucleic acid molecule describe herein is a double-stranded siRNA molecule that down-regulates expression of at least one of DMD, DUX4, DYSF, EMD, or LMNA, wherein each strand of the siRNA molecule comprises about 19 to about 23 nucleotides, and wherein each strand comprises at least about 19 nucleotides that are complementary to the nucleotides of the other strand. In some instances, the RNAi activity occurs within a cell. In other instances, the RNAi activity occurs in a reconstituted in vitro system.

In some aspects, a polynucleic acid molecule describe herein has RNAi activity that modulates expression of RNA encoded by the DMD gene. In some instances, a polynucleic acid molecule describe herein is a single-stranded siRNA molecule that down-regulates expression of DMD, wherein the single-stranded siRNA molecule comprises a nucleotide sequence that is complementary to a nucleotide sequence of DMD or RNA encoded by DMD or a portion thereof.

In some cases, a polynucleic acid molecule describe herein is a single-stranded siRNA molecule that down-regulates expression of DMD, wherein the siRNA molecule comprises about 15 to 25, 18 to 24, or 19 to about 23 nucleotides. In some cases, a polynucleic acid molecule describe herein is a single-stranded siRNA molecule that down-regulates expression of DMD, wherein the siRNA molecule comprises about 19 to about 23 nucleotides. In some instances, the RNAi activity occurs within a cell. In other instances, the RNAi activity occurs in a reconstituted in vitro system.

In some instances, the polynucleic acid molecule is a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. In some instances, the polynucleic acid molecule is assembled from two separate polynucleotides, where one strand is the sense strand and the other is the antisense strand, wherein the antisense and sense strands are self-complementary (e.g., each strand comprises nucleotide sequence that is complementary to nucleotide sequence in the other strand; such as where the antisense strand and sense strand form a duplex or double stranded structure, for example wherein the double stranded region is about 19, 20, 21, 22, 23, or more base pairs); the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof. Alternatively, the polynucleic acid molecule is assembled from a single oligonucleotide, where the self-complementary sense and antisense regions of the polynucleic acid molecule are linked by means of a nucleic acid based or non-nucleic acid-based linker(s).

In some cases, the polynucleic acid molecule is a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. In other cases, the polynucleic acid molecule is a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide is processed either in vivo or in vitro to generate an active polynucleic acid molecule capable of mediating RNAi. In additional cases, the polynucleic acid molecule also comprises a single stranded polynucleotide having nucleotide sequence complementary to nucleotide sequence in a target nucleic acid molecule or a portion thereof (for example, where such polynucleic acid molecule does not require the presence within the polynucleic acid molecule of nucleotide sequence corresponding to the target nucleic acid sequence or a portion thereof), wherein the single stranded polynucleotide further comprises a terminal phosphate group, such as a 5′-phosphate (see for example Martinez et al., 2002, Cell., 110, 563-574 and Schwarz et al., 2002, Molecular Cell, 10, 537-568), or 5′,3′-diphosphate.

In some instances, an asymmetric is a linear polynucleic acid molecule comprising an antisense region, a loop portion that comprises nucleotides or non-nucleotides, and a sense region that comprises fewer nucleotides than the antisense region to the extent that the sense region has enough complimentary nucleotides to base pair with the antisense region and form a duplex with loop. For example, an asymmetric hairpin polynucleic acid molecule comprises an antisense region having length sufficient to mediate RNAi in a cell or in vitro system (e.g. about 19 to about 22 nucleotides) and a loop region comprising about 4 to about 8 nucleotides, and a sense region having about 3 to about 18 nucleotides that are complementary to the antisense region. In some cases, the asymmetric hairpin polynucleic acid molecule also comprises a 5′-terminal phosphate group that is chemically modified. In additional cases, the loop portion of the asymmetric hairpin polynucleic acid molecule comprises nucleotides, non-nucleotides, linker molecules, or conjugate molecules.

In some aspects, an asymmetric duplex is a polynucleic acid molecule having two separate strands comprising a sense region and an antisense region, wherein the sense region comprises fewer nucleotides than the antisense region to the extent that the sense region has enough complimentary nucleotides to base pair with the antisense region and form a duplex. For example, an asymmetric duplex polynucleic acid molecule comprises an antisense region having length sufficient to mediate RNAi in a cell or in vitro system (e.g. about 19 to about 22 nucleotides) and a sense region having about 3 to about 18 nucleotides that are complementary to the antisense region.

In some cases, an universal base refers to nucleotide base analogs that form base pairs with each of the natural DNA/RNA bases with little discrimination between them. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art (see for example Loakes, 2001, Nucleic Acids Research, 29, 2437-2447).

### Polynucleic Acid Molecule Synthesis

In some aspects, a polynucleic acid molecule described herein is constructed using chemical synthesis and/or enzymatic ligation reactions using procedures known in the art. For example, a polynucleic acid molecule is chemically synthesized using naturally occurring nucleotides or variously modified nucleotides designed to increase the biological stability of the molecules or to increase the physical stability of the duplex formed between the polynucleic acid molecule and target nucleic acids. Exemplary methods include those described in: U.S. Pat. Nos. 5,142,047; 5,185,444; 5,889,136; 6,008,400; and 6,111,086; PCT Publication No. WO2009099942; or European Publication No. 1579015. Additional exemplary methods include those described in: Griffey et al., “2′-O-aminopropyl ribonucleotides: a zwitterionic modification that enhances the exonuclease resistance and biological activity of antisense oligonucleotides,” J. Med. Chem. 39(26):5100-5109 (1997)); Obika, et al. “Synthesis of 2′-0,4′-C-methyleneuridine and -cytidine. Novel bicyclic nucleosides having a fixed C3, -endo sugar puckering”. Tetrahedron Letters 38 (50): 8735 (1997); Koizumi, M. “ENA oligonucleotides as therapeutics”. Current opinion in molecular therapeutics 8 (2): 144-149 (2006); and Abramova et al., “Novel oligonucleotide analogues based on morpholino nucleoside subunits-antisense technologies: new chemical possibilities,” Indian Journal of Chemistry 48B:1721-1726 (2009). Alternatively, the polynucleic acid molecule is produced biologically using an expression vector into which a polynucleic acid molecule has been subcloned in an antisense orientation (i.e., RNA transcribed from the inserted polynucleic acid molecule will be of an antisense orientation to a target polynucleic acid molecule of interest).

In some aspects, a polynucleic acid molecule is synthesized via a tandem synthesis methodology, wherein both strands are synthesized as a single contiguous oligonucleotide fragment or strand separated by a cleavable linker which is subsequently cleaved to provide separate fragments or strands that hybridize and permit purification of the duplex.

In some instances, a polynucleic acid molecule is also assembled from two distinct nucleic acid strands or fragments wherein one fragment includes the sense region and the second fragment includes the antisense region of the molecule.

Additional modification methods for incorporating, for example, sugar, base and phosphate modifications include: Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic Acid Sciences), 48, 39-55; Verma and Eckstein, 1998, Annu. Rev. Biochem., 67, 99-134; and Burlina et al., 1997, Bioorg. Med. Chem., 5, 1999-2010. Such publications describe general methods and strategies to determine the location of incorporation of sugar, base and/or phosphate modifications and the like into nucleic acid molecules without modulating catalysis.

In some instances, while chemical modification of the polynucleic acid molecule intemucleotide linkages with phosphorothioate, phosphorodithioate, and/or 5′-methylphosphonate linkages improves stability, excessive modifications sometimes cause toxicity or decreased activity. Therefore, when designing nucleic acid molecules, the amount of these internucleotide linkages in some cases is minimized. In such cases, the reduction in the concentration of these linkages lowers toxicity, increases efficacy and higher specificity of these molecules.

### Nucleic Acid-Polypeptide Conjugate

In some aspects, a polynucleic acid molecule is further conjugated to a polypeptide A for delivery to a site of interest. In some cases, a polynucleic acid molecule is conjugated to a polypeptide A and optionally a polymeric moiety.

In some instances, at least one polypeptide A is conjugated to at least one B. In some instances, the at least one polypeptide A is conjugated to the at least one B to form an A-B conjugate. In some aspects, at least one A is conjugated to the 5′ terminus of B, the 3′ terminus of B, an internal site on B, or in any combinations thereof. In some instances, the at least one polypeptide A is conjugated to at least two B. In some instances, the at least one polypeptide A is conjugated to at least 2, 3, 4, 5, 6, 7, 8, or more B.

In some aspects, at least one polypeptide A is conjugated at one terminus of at least one B while at least one C is conjugated at the opposite terminus of the at least one B to form an A-B-C conjugate. In some instances, at least one polypeptide A is conjugated at one terminus of the at least one B while at least one of C is conjugated at an internal site on the at least one B. In some instances, at least one polypeptide A is conjugated directly to the at least one C. In some instances, the at least one B is conjugated indirectly to the at least one polypeptide A via the at least one C to form an A-C-B conjugate.

In some instances, at least one B and/or at least one C, and optionally at least one D are conjugated to at least one polypeptide A. In some instances, the at least one B is conjugated at a terminus (e.g., a 5′ terminus or a 3′ terminus) to the at least one polypeptide A or are conjugated via an internal site to the at least one polypeptide A. In some cases, the at least one C is conjugated either directly to the at least one polypeptide A or indirectly via the at least one B. If indirectly via the at least one B, the at least one C is conjugated either at the same terminus as the at least one polypeptide A on B, at opposing terminus from the at least one polypeptide A, or independently at an internal site. In some instances, at least one additional polypeptide A is further conjugated to the at least one polypeptide A, to B, or to C. In additional instances, the at least one D is optionally conjugated either directly or indirectly to the at least one polypeptide A, to the at least one B, or to the at least one C. If directly to the at least one polypeptide A, the at least one D is also optionally conjugated to the at least one B to form an A-D-B conjugate or is optionally conjugated to the at least one B and the at least one C to form an A-D-B-C conjugate. In some instances, the at least one D is directly conjugated to the at least one polypeptide A and indirectly to the at least one B and the at least one C to form a D-A-B-C conjugate. If indirectly to the at least one polypeptide A, the at least one D is also optionally conjugated to the at least one B to form an A-B-D conjugate or is optionally conjugated to the at least one B and the at least one C to form an A-B-D-C conjugate. In some instances, at least one additional D is further conjugated to the at least one polypeptide A, to B, or to C.

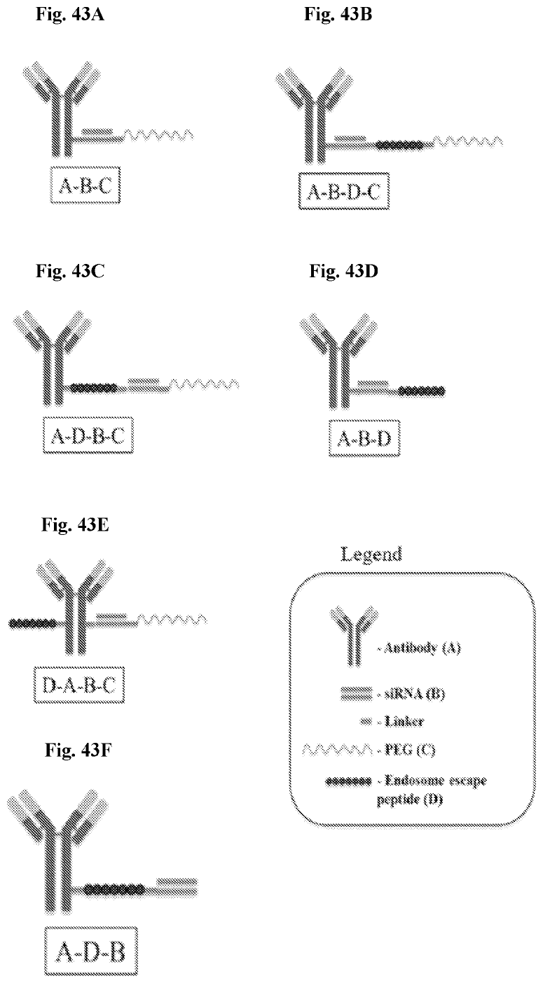

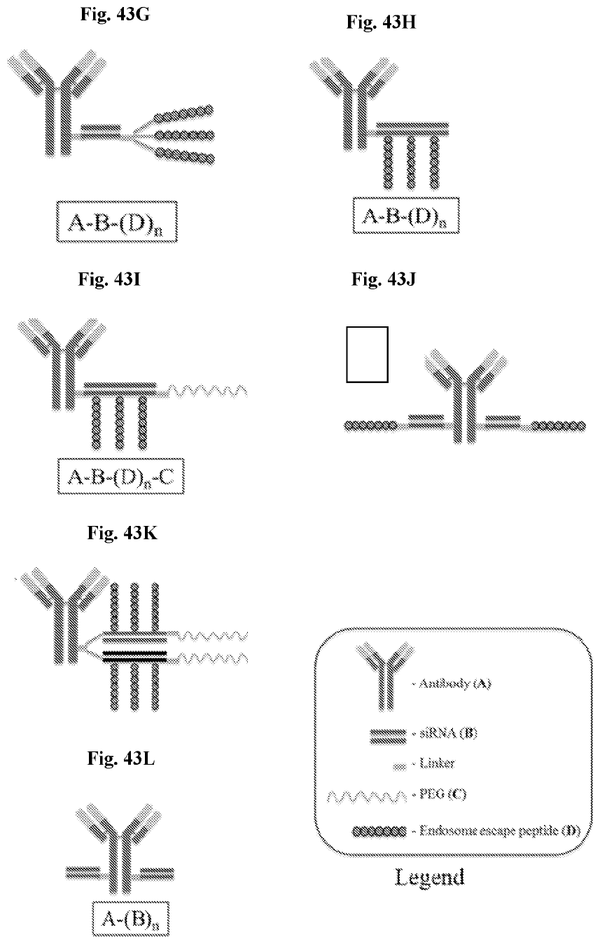

In some aspects, a polynucleic acid molecule conjugate comprises a construct as illustrated in FIG. 43A-FIG. 43L.

The antibody as illustrated in FIG. 43A-FIG. 43L is for representation purposes only and encompasses a humanized antibody or binding fragment thereof, chimeric antibody or binding fragment thereof, monoclonal antibody or binding fragment thereof, monovalent Fab′, divalent Fab2, single-chain variable fragment (scFv), diabody, minibody, nanobody, single-domain antibody (sdAb), or camelid antibody or binding fragment thereof.

### Antibody-Peptide-Oligonucleotide Conjugate (APOC)

In some aspects, the antibody-peptide-oligonucleotide conjugate is an antibody peptide-PMO conjugate. In some aspects the antibody-peptide-PMO (ADB or ADB-PEG) comprises:
i. Antibody=anti-mouse TfR1 (mTfR1) mAb;
ii. Peptide=R8 (SEQ ID NO: 1016), (RXR)4XB (SEQ ID NO: 1065), or RXRRBRRXRYQFLIRXRBRXRB (SEQ ID NO: 1067) (pip6a) where X=6-aminohexanoic acid and B=beta-alanine. Peptides may also contain a Valine-Citrulline (ValCit) dipeptide at the beginning of the sequence;
iii. PMO=3′NH2-mouse dystrophin exon 23 skipping PMO (sequence: GGCCAAACCTCGGCTTACCTGAAAT (SEQ ID NO:28)) (mEx23PMO);
iv. Linker=6-maleimidocaroic acid (MC).
v. The ADB configuration allows the peptide to be between the antibody and the oligo.

This can result in steric shielding of the charged peptide amino acids and prevent off target binding. However, once cleaved from the antibody, a peptide modified PMO (PPMO) will be generated which can escape the endosome and perform its function. Without the peptide, the PMO accumulated in endosomes and shows 20-fold less potency as a result.

In some aspects, the antibody-peptide-PMO (ADB or ADB-PEG) comprises the generic structure: Protein-Cys-Maleimide-linker-peptide-PMO

In some aspects, the antibody-peptide-PMO (ADB or ADB-PEG) comprises the specific molecule structure:
i. mTfR1-(Cys-MC-R8-mEx23PMO)n
ii. mTfR1-(Cys-MC-(RXR)4XB-mEx23PMO)n
iii. mTfR1-(Cys-MC-Pip6a-mEx23PMO)n
iv. mTfR1-(Cys-MC-ValCit-R8-mEx23PMO)n
v. mTfR1-(Cys-MC-ValCit-(RXR)4XB-mEx23PMO)n
vi. mTfR1-(Cys-MC-ValCit-Pip6a-mEx23PMO)n
wherein n is an integer ≥1.

In some aspects the antibody-peptide-PMO (ADB or ADB-PEG) comprises the following alternatives:
i. Other antibodies
   1. anti-human TfR1 antibodies will replace mTfR1 for therapeutics.
ii. Fab-PPMO conjugates
   2. Antibodies could also be the Fab with the same target. Fabs can have some benefits over mAbs, including reducing the protein burden, decreasing sample heterogeneity, and abolishing effector function.
iii. Different drug-antibody ratios (DAR)
   3. DAR could be a mix with a different average (example: average DAR2 vs average DAR4) or individual DAR species. DAR can influence the compound activity. For example, higher DARs will have more peptides, thus higher positive charge, and potentially leading to higher off target binding, and lower activity. However, if this challenge is overcome, higher DAR could result in higher payload delivery per antibody, reducing the protein burden.
iv. Different peptides
   4. Initial peptides tested are shown in the literature to be highly functional as PPMOs. However, they may not necessarily be the best peptides for antibody-PPMOs. Thus, the peptides listed above could be replaced by any peptide associated with cell penetration/endosomal escape.
v. Different PMOs
   5. Initial compounds have mEx23PMO to demonstrate proof of concept in animal models. However, other iterations will include human specific exon skipping oligonucleotides.
   6. PMOs can also contain other conjugation handles on either the 3′ or 5′ ends of the molecule. This allows for other chemical approaches to conjugation if needed for specific payloads.
vi. Different Linkers
   7. Other linkers will include cleavable or non-cleavable linkers which can have an effect on activity. Cleavable linkers may be advantageous as cleavage from the antibody will release a PPMO which are highly potent.
   8. Branched linkers can also be used to introduce a new chemical moiety to effect activity. PEG inclusion with the molecule could result in more steric protection of the charged peptide, decreasing off target binding (ACB-PEG).

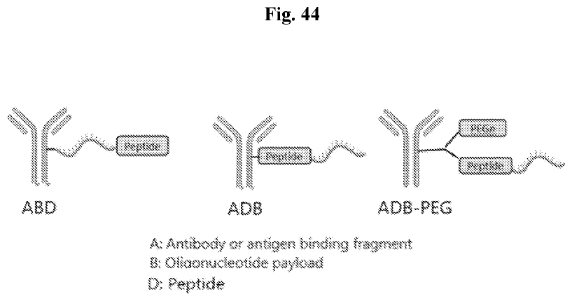

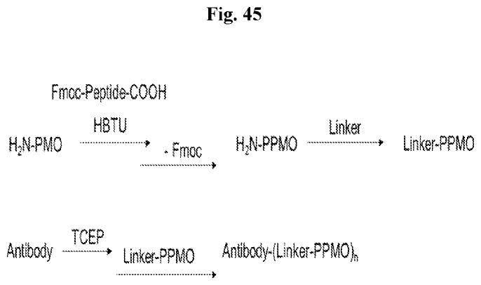

FIG. 44 illustrates possible configurations of the antibody/Fab (A), oligo payload (B)_and peptide (D). FIG. 45 illustrates general synthetic strategy used to synthesize future AOC-PPMOs (ADB).

### Binding Moiety

In some aspects, the binding moiety A is a polypeptide. In some instances, the polypeptide is an antibody or its fragment thereof. In some cases, the fragment is a binding fragment. In some instances, the antibody or binding fragment thereof comprises a humanized antibody or binding fragment thereof, murine antibody or binding fragment thereof, chimeric antibody or binding fragment thereof, monoclonal antibody or binding fragment thereof, monovalent Fab′, divalent Fab2, F(ab)′3 fragments, single-chain variable fragment (scFv), bis-scFv, (scFv)2, diabody, minibody, nanobody, triabody, tetrabody, disulfide stabilized Fv protein (dsFv), single-domain antibody (sdAb), Ig NAR, camelid antibody or binding fragment thereof, bispecific antibody or biding fragment thereof, or a chemically modified derivative thereof.

In some instances, A is an antibody or binding fragment thereof. In some instances, A is a humanized antibody or binding fragment thereof, murine antibody or binding fragment thereof, chimeric antibody or binding fragment thereof, monoclonal antibody or binding fragment thereof, monovalent Fab′, divalent Fab2, F(ab)′3 fragments, single-chain variable fragment (scFv), bis-scFv, (scFv)2, diabody, minibody, nanobody, triabody, tetrabody, disulfide stabilized Fv protein (“dsFv”), single-domain antibody (sdAb), Ig NAR, camelid antibody or binding fragment thereof, bispecific antibody or biding fragment thereof, or a chemically modified derivative thereof. In some instances, A is a humanized antibody or binding fragment thereof. In some instances, A is a murine antibody or binding fragment thereof. In some instances, A is a chimeric antibody or binding fragment thereof. In some instances, A is a monoclonal antibody or binding fragment thereof. In some instances, A is a monovalent Fab′. In some instances, A is a divalent Fab2. In some instances, A is a single-chain variable fragment (scFv).

In some aspects, the binding moiety A is a bispecific antibody or binding fragment thereof. In some instances, the bispecific antibody is a trifunctional antibody or a bispecific mini-antibody. In some cases, the bispecific antibody is a trifunctional antibody. In some instances, the trifunctional antibody is a full length monoclonal antibody comprising binding sites for two different antigens.

In some cases, the bispecific antibody is a bispecific mini-antibody. In some instances, the bispecific mini-antibody comprises divalent Fab2, F(ab)′3 fragments, bis-scFv, (scFv)2, diabody, minibody, triabody, tetrabody or a bi-specific T-cell engager (BiTE). In some aspects, the bi-specific T-cell engager is a fusion protein that contains two single-chain variable fragments (scFvs) in which the two scFvs target epitopes of two different antigens.

In some aspects, the binding moiety A is a bispecific mini-antibody. In some instances, A is a bispecific Fab2. In some instances, A is a bispecific F(ab)′3 fragment. In some cases, A is a bispecific bis-scFv. In some cases, A is a bispecific (scFv)2. In some aspects, A is a bispecific diabody. In some aspects, A is a bispecific minibody. In some aspects, A is a bispecific triabody. In other aspects, A is a bispecific tetrabody. In other aspects, A is a bi-specific T-cell engager (BiTE).

In some aspects, the binding moiety A is a trispecific antibody. In some instances, the trispecific antibody comprises F(ab)′3 fragments or a triabody. In some instances, A is a trispecific F(ab)′3 fragment. In some cases, A is a triabody. In some aspects, A is a trispecific antibody as described in Dimas, et al., “Development of a trispecific antibody designed to simultaneously and efficiently target three different antigens on tumor cells,” Mol. Pharmaceutics, 12(9): 3490-3501 (2015).

In some aspects, the binding moiety A is an antibody or antigen binding fragment thereof that recognizes a cell surface protein. In some instances, the binding moiety A is an antibody or binding fragment thereof that recognizes a cell surface protein on a muscle cell. Exemplary cell surface proteins recognized by an antibody or binding fragment thereof include, but are not limited to, Sca-1, CD34, Myo-D, myogenin, MRF4, NCAM, CD43, and CD95 (Fas).

In some instances, the cell surface protein comprises clusters of differentiation (CD) cell surface markers. Exemplary CD cell surface markers include, but are not limited to, CD1, CD2, CD3, CD4, CD5, CD6, CD7, CD8, CD9, CD10, CD11a, CD11b, CD11c, CD11d, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L (L-selectin), CD62P, CD63, CD64, CD65, CD66a, CD66b, CD66c, CD66d, CD66e, CD79 (e.g., CD79a, CD79b), CD90, CD95 (Fas), CD103, CD104, CD125 (IL5RA), CD134 (OX40), CD137 (4-1BB), CD152 (CTLA-4), CD221, CD274, CD279 (PD-1), CD319 (SLAMF7), CD326 (EpCAM), and the like.

In some instances, the binding moiety A is an antibody or antigen binding fragment thereof that recognizes a CD cell surface marker. In some instances, the binding moiety A is an antibody or binding fragment thereof that recognizes CD1, CD2, CD3, CD4, CD5, CD6, CD7, CD8, CD9, CD10, CD11a, CD11b, CD11c, CD11d, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L (L-selectin), CD62P, CD63, CD64, CD65, CD66a, CD66b, CD66c, CD66d, CD66e, CD79 (e.g., CD79a, CD79b), CD90, CD95 (Fas), CD103, CD104, CD125 (IL5RA), CD134 (OX40), CD137 (4-1BB), CD152 (CTLA-4), CD221, CD274, CD279 (PD-1), CD319 (SLAMF7), CD326 (EpCAM), or a combination thereof.

In some aspects, the binding moiety A is conjugated to a polynucleic acid molecule (B) non-specifically. In some instances, the binding moiety A is conjugated to a polynucleic acid molecule (B) via a lysine residue or a cysteine residue, in a non-site specific manner. In some instances, the binding moiety A is conjugated to a polynucleic acid molecule (B) via a lysine residue in a non-site specific manner. In some cases, the binding moiety A is conjugated to a polynucleic acid molecule (B) via a cysteine residue in a non-site specific manner.

In some aspects, the binding moiety A is conjugated to a polynucleic acid molecule (B) in a site-specific manner. In some instances, the binding moiety A is conjugated to a polynucleic acid molecule (B) through a lysine residue, a cysteine residue, at the 5′-terminus, at the 3′-terminus, an unnatural amino acid, or an enzyme-modified or enzyme-catalyzed residue, via a site-specific manner. In some instances, the binding moiety A is conjugated to a polynucleic acid molecule (B) through a lysine residue via a site-specific manner. In some instances, the binding moiety A is conjugated to a polynucleic acid molecule (B) through a cysteine residue via a site-specific manner. In some instances, the binding moiety A is conjugated to a polynucleic acid molecule (B) at the 5′-terminus via a site-specific manner. In some instances, the binding moiety A is conjugated to a polynucleic acid molecule (B) at the 3′-terminus via a site-specific manner.

In some instances, the binding moiety A is conjugated to a polynucleic acid molecule (B) through an unnatural amino acid via a site-specific manner. In some instances, the binding moiety A is conjugated to a polynucleic acid molecule (B) through an enzyme-modified or enzyme-catalyzed residue via a site-specific manner.

In some aspects, one or more polynucleic acid molecule (B) is conjugated to a binding moiety A. In some instances, about 1, 2, 3, 4, 5, 6, 7, 8, 9, 10, 11, 12, 13, 14, 15, 16, or more polynucleic acid molecules are conjugated to one binding moiety A. In some instances, about 1 polynucleic acid molecule is conjugated to one binding moiety A. In some instances, about 2 polynucleic acid molecules are conjugated to one binding moiety A. In some instances, about 3 polynucleic acid molecules are conjugated to one binding moiety A. In some instances, about 4 polynucleic acid molecules are conjugated to one binding moiety A. In some instances, about 5 polynucleic acid molecules are conjugated to one binding moiety A. In some instances, about 6 polynucleic acid molecules are conjugated to one binding moiety A. In some instances, about 7 polynucleic acid molecules are conjugated to one binding moiety A. In some instances, about 8 polynucleic acid molecules are conjugated to one binding moiety A. In some instances, about 9 polynucleic acid molecules are conjugated to one binding moiety A. In some instances, about 10 polynucleic acid molecules are conjugated to one binding moiety A. In some instances, about 11 polynucleic acid molecules are conjugated to one binding moiety A. In some instances, about 12 polynucleic acid molecules are conjugated to one binding moiety A. In some instances, about 13 polynucleic acid molecules are conjugated to one binding moiety A. In some instances, about 14 polynucleic acid molecules are conjugated to one binding moiety A. In some instances, about 15 polynucleic acid molecules are conjugated to one binding moiety A. In some instances, about 16 polynucleic acid molecules are conjugated to one binding moiety A. In some cases, the one or more polynucleic acid molecules are the same. In other cases, the one or more polynucleic acid molecules are different.

In some aspects, the number of polynucleic acid molecule (B) conjugated to a binding moiety A forms a ratio. In some instances, the ratio is referred to as a DAR (drug-to-antibody) ratio, in which the drug as referred to herein is the polynucleic acid molecule (B). In some instances, the DAR ratio of the polynucleic acid molecule (B) to binding moiety A is about 1, 2, 3, 4, 5, 6, 7, 8, 9, 10, 11, 12, 13, 14, 15, 16, or greater. In some instances, the DAR ratio of the polynucleic acid molecule (B) to binding moiety A is about 1 or greater. In some instances, the DAR ratio of the polynucleic acid molecule (B) to binding moiety A is about 2 or greater. In some instances, the DAR ratio of the polynucleic acid molecule (B) to binding moiety A is about 3 or greater. In some instances, the DAR ratio of the polynucleic acid molecule (B) to binding moiety A is about 4 or greater. In some instances, the DAR ratio of the polynucleic acid molecule (B) to binding moiety A is about 5 or greater. In some instances, the DAR ratio of the polynucleic acid molecule (B) to binding moiety A is about 6 or greater. In some instances, the DAR ratio of the polynucleic acid molecule (B) to binding moiety A is about 7 or greater. In some instances, the DAR ratio of the polynucleic acid molecule (B) to binding moiety A is about 8 or greater. In some instances, the DAR ratio of the polynucleic acid molecule (B) to binding moiety A is about 9 or greater. In some instances, the DAR ratio of the polynucleic acid molecule (B) to binding moiety A is about 10 or greater. In some instances, the DAR ratio of the polynucleic acid molecule (B) to binding moiety A is about 11 or greater. In some instances, the DAR ratio of the polynucleic acid molecule (B) to binding moiety A is about 12 or greater.

In some instances, the DAR ratio of the polynucleic acid molecule (B) to binding moiety A is about 1, 2, 3, 4, 5, 6, 7, 8, 9, 10, 11, 12, 13, 14, 15, or 16. In some instances, the DAR ratio of the polynucleic acid molecule (B) to binding moiety A is about 1. In some instances, the DAR ratio of the polynucleic acid molecule (B) to binding moiety A is about 2. In some instances, the DAR ratio of the polynucleic acid molecule (B) to binding moiety A is about 3. In some instances, the DAR ratio of the polynucleic acid molecule (B) to binding moiety A is about 4. In some instances, the DAR ratio of the polynucleic acid molecule (B) to binding moiety A is about 5. In some instances, the DAR ratio of the polynucleic acid molecule (B) to binding moiety A is about 6. In some instances, the DAR ratio of the polynucleic acid molecule (B) to binding moiety A is about 7. In some instances, the DAR ratio of the polynucleic acid molecule (B) to binding moiety A is about 8. In some instances, the DAR ratio of the polynucleic acid molecule (B) to binding moiety A is about 9. In some instances, the DAR ratio of the polynucleic acid molecule (B) to binding moiety A is about 10. In some instances, the DAR ratio of the polynucleic acid molecule (B) to binding moiety A is about 11. In some instances, the DAR ratio of the polynucleic acid molecule (B) to binding moiety A is about 12. In some instances, the DAR ratio of the polynucleic acid molecule (B) to binding moiety A is about 13. In some instances, the DAR ratio of the polynucleic acid molecule (B) to binding moiety A is about 14. In some instances, the DAR ratio of the polynucleic acid molecule (B) to binding moiety A is about 15. In some instances, the DAR ratio of the polynucleic acid molecule (B) to binding moiety A is about 16.

In some instances, the DAR ratio of the polynucleic acid molecule (B) to binding moiety A is 1, 2, 3, 4, 5, 6, 7, 8, 9, 10, 11, 12, 13, 14, 15, or 16. In some instances, the DAR ratio of the polynucleic acid molecule (B) to binding moiety A is 1. In some instances, the DAR ratio of the polynucleic acid molecule (B) to binding moiety A is 2. In some instances, the DAR ratio of the polynucleic acid molecule (B) to binding moiety A is 4. In some instances, the DAR ratio of the polynucleic acid molecule (B) to binding moiety A is 6. In some instances, the DAR ratio of the polynucleic acid molecule (B) to binding moiety A is 8. In some instances, the DAR ratio of the polynucleic acid molecule (B) to binding moiety A is 12.

In some instances, a conjugate comprising polynucleic acid molecule (B) and binding moiety A has improved activity as compared to a conjugate comprising polynucleic acid molecule (B) without a binding moiety A. In some instances, improved activity results in enhanced biologically relevant functions, e.g., improved stability, affinity, binding, functional activity, and efficacy in treatment or prevention of a disease state. In some instances, the disease state is a result of one or more mutated exons of a gene. In some instances, the conjugate comprising polynucleic acid molecule (B) and binding moiety A results in increased exon skipping of the one or more mutated exons as compared to the conjugate comprising polynucleic acid molecule (B) without a binding moiety A. In some instances, exon skipping is increased by at least or about 5%, 10%, 20%, 25%, 30%, 40%, 50%, 60%, 70%, 80%, 90%, 95%, or more than 95% in the conjugate comprising polynucleic acid molecule (B) and binding moiety A as compared to the conjugate comprising polynucleic acid molecule (B) without a binding moiety A.

In some aspects, an antibody or its binding fragment is further modified using conventional techniques known in the art, for example, by using amino acid deletion, insertion, substitution, addition, and/or by recombination and/or any other modification (e.g. posttranslational and chemical modifications, such as glycosylation and phosphorylation) known in the art either alone or in combination. In some instances, the modification further comprises a modification for modulating interaction with Fc receptors. In some instances, the one or more modifications include those described in, for example, International Publication No.

WO97/34631, which discloses amino acid residues involved in the interaction between the Fc domain and the FcRn receptor. Methods for introducing such modifications in the nucleic acid sequence underlying the amino acid sequence of an antibody or its binding fragment is well known to the person skilled in the art.

In some instances, an antibody binding fragment further encompasses its derivatives and includes polypeptide sequences containing at least one CDR.

In some instances, the term “single-chain” as used herein means that the first and second domains of a bi-specific single chain construct are covalently linked, preferably in the form of a co-linear amino acid sequence encodable by a single nucleic acid molecule.

In some instances, a bispecific single chain antibody construct relates to a construct comprising two antibody derived binding domains. In such aspects, bi-specific single chain antibody construct is tandem bi-scFv or diabody. In some instances, a scFv contains a VH and VL domain connected by a linker peptide. In some instances, linkers are of a length and sequence sufficient to ensure that each of the first and second domains can, independently from one another, retain their differential binding specificities.

In some aspects, binding to or interacting with as used herein defines a binding/interaction of at least two antigen-interaction-sites with each other. In some instances, antigen-interaction-site defines a motif of a polypeptide that shows the capacity of specific interaction with a specific antigen or a specific group of antigens. In some cases, the binding/interaction is also understood to define a specific recognition. In such cases, specific recognition refers to that the antibody or its binding fragment is capable of specifically interacting with and/or binding to at least two amino acids of each of a target molecule. For example, specific recognition relates to the specificity of the antibody molecule, or to its ability to discriminate between the specific regions of a target molecule. In additional instances, the specific interaction of the antigen-interaction-site with its specific antigen results in an initiation of a signal, e.g. due to the induction of a change of the conformation of the antigen, an oligomerization of the antigen, etc. In further aspects, the binding is exemplified by the specificity of a “key-lock-principle”. Thus in some instances, specific motifs in the amino acid sequence of the antigen-interaction-site and the antigen bind to each other as a result of their primary, secondary or tertiary structure as well as the result of secondary modifications of said structure. In such cases, the specific interaction of the antigen-interaction-site with its specific antigen results as well in a simple binding of the site to the antigen.

In some instances, specific interaction further refers to a reduced cross-reactivity of the antibody or its binding fragment or a reduced off-target effect. For example, the antibody or its binding fragment that bind to the polypeptide/protein of interest but do not or do not essentially bind to any of the other polypeptides are considered as specific for the polypeptide/protein of interest. Examples for the specific interaction of an antigen-interaction-site with a specific antigen comprise the specificity of a ligand for its receptor, for example, the interaction of an antigenic determinant (epitope) with the antigenic binding site of an antibody.

### Conjugation Chemistry

In some aspects, a polynucleic acid molecule B is conjugated to a binding moiety. In some instances, the binding moiety comprises amino acids, peptides, polypeptides, proteins, antibodies, antigens, toxins, hormones, lipids, nucleotides, nucleosides, sugars, carbohydrates, polymers such as polyethylene glycol and polypropylene glycol, as well as analogs or derivatives of all of these classes of substances. Additional examples of binding moiety also include steroids, such as cholesterol, phospholipids, di- and triacylglycerols, fatty acids, hydrocarbons (e.g., saturated, unsaturated, or contains substitutions), enzyme substrates, biotin, digoxigenin, and polysaccharides. In some instances, the binding moiety is an antibody or binding fragment thereof. In some instances, the polynucleic acid molecule is further conjugated to a polymer, and optionally an endosomolytic moiety.

In some aspects, the polynucleic acid molecule is conjugated to the binding moiety by a chemical ligation process. In some instances, the polynucleic acid molecule is conjugated to the binding moiety by a native ligation. In some instances, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology.,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some instances, the conjugation is as described in U.S. Pat. No. 8,936,910. In some aspects, the polynucleic acid molecule is conjugated to the binding moiety either site-specifically or non-specifically via native ligation chemistry.

In some instances, the polynucleic acid molecule is conjugated to the binding moiety by a site-directed method utilizing a “traceless” coupling technology (Philochem). In some instances, the “traceless” coupling technology utilizes an N-terminal 1,2-aminothiol group on the binding moiety which is then conjugate with a polynucleic acid molecule containing an aldehyde group. (see Casi et al., “Site-specific traceless coupling of potent cytotoxic drugs to recombinant antibodies for pharmacodelivery,” JACS 134(13): 5887-5892 (2012))

In some instances, the polynucleic acid molecule is conjugated to the binding moiety by a site-directed method utilizing an unnatural amino acid incorporated into the binding moiety. In some instances, the unnatural amino acid comprises p-acetylphenylalanine (pAcPhe). In some instances, the keto group of pAcPhe is selectively coupled to an alkoxy-amine derivatived conjugating moiety to form an oxime bond. (see Axup et al., “Synthesis of site-specific antibody-drug conjugates using unnatural amino acids,” PNAS 109(40): 16101-16106 (2012)).

In some instances, the polynucleic acid molecule is conjugated to the binding moiety by a site-directed method utilizing an enzyme-catalyzed process. In some instances, the site-directed method utilizes SMARTag™ technology (Redwood). In some instances, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized polynucleic acid molecule via hydrazino-Picte2′-Spengler (HIPS) ligation. (see Wu et al., “Site-specific chemical modification of recombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013))

In some instances, the enzyme-catalyzed process comprises microbial transglutaminase (mTG). In some cases, the polynucleic acid molecule is conjugated to the binding moiety utilizing a microbial transglutaminze catalyzed process. In some instances, mTG catalyzes the formation of a covalent bond between the amide side chain of a glutamine within the recognition sequence and a primary amine of a functionalized polynucleic acid molecule. In some instances, mTG is produced from Streptomyces mobarensis. (see Strop et al., “Location matters: site of conjugation modulates stability and pharmacokinetics of antibody drug conjugates,” Chemistry and Biology 20(2) 161-167 (2013))

In some instances, the polynucleic acid molecule is conjugated to the binding moiety by a method as described in PCT Publication No. WO2014/140317, which utilizes a sequence-specific transpeptidase.

In some instances, the polynucleic acid molecule is conjugated to the binding moiety by a method as described in U.S. Patent Publication Nos. 2015/0105539 and 2015/0105540.

### Production of Antibodies or Binding Fragments Thereof

In some aspects, polypeptides described herein (e.g., antibodies and its binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its antigen binding fragment is optionally generated by immunizing an animal, such as a rabbit, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some aspects, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region, e.g., humanized antibodies.

In some aspects, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some aspects, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific aspects, the expression of the antibody is regulated by a constitutive, an inducible or a tissue, specific promoter.

In some aspects, a variety of host-expression vector systems is utilized to express an antibody or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk-, hgprt- or aprt-cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification or analysis of an antibody or antibody conjugates is used, for example, by chromatography (e.g., ion exchange, affinity, particularly by affinity for the specific antigen after Protein A, and sizing column chromatography), centrifugation, differential solubility, or by any other standard technique for the purification of proteins. Exemplary chromatography methods included, but are not limited to, strong anion exchange chromatography, hydrophobic interaction chromatography, size exclusion chromatography, and fast protein liquid chromatography.

### Polymer Conjugating Moiety

In some aspects, a polymer moiety C is further conjugated to a polynucleic acid molecule described herein, a binding moiety described herein, or in combinations thereof. In some instances, a polymer moiety C is conjugated a polynucleic acid molecule. In some cases, a polymer moiety C is conjugated to a binding moiety. In other cases, a polymer moiety C is conjugated to a polynucleic acid molecule-binding moiety molecule. In additional cases, a polymer moiety C is conjugated, as illustrated supra.

In some instances, the polymer moiety C is a natural or synthetic polymer, consisting of long chains of branched or unbranched monomers, and/or cross-linked network of monomers in two or three dimensions. In some instances, the polymer moiety C includes a polysaccharide, lignin, rubber, or polyalkylen oxide (e.g., polyethylene glycol). In some instances, the at least one polymer moiety C includes, but is not limited to, alpha-, omega-dihydroxylpolyethyleneglycol, biodegradable lactone-based polymer, e.g. polyacrylic acid, polylactide acid (PLA), poly(glycolic acid) (PGA), polypropylene, polystyrene, polyolefin, polyamide, polycyanoacrylate, polyimide, polyethylenterephthalat (PET, PETG), polyethylene terephthalate (PETE), polytetramethylene glycol (PTG), or polyurethane as well as mixtures thereof. As used herein, a mixture refers to the use of different polymers within the same compound as well as in reference to block copolymers. In some cases, block copolymers are polymers wherein at least one section of a polymer is build up from monomers of another polymer. In some instances, the polymer moiety C comprises polyalkylene oxide. In some instances, the polymer moiety C comprises PEG. In some instances, the polymer moiety C comprises polyethylene imide (PEI) or hydroxy ethyl starch (HES).

In some instances, C is a PEG moiety. In some instances, the PEG moiety is conjugated at the 5′ terminus of the polynucleic acid molecule while the binding moiety is conjugated at the 3′ terminus of the polynucleic acid molecule. In some instances, the PEG moiety is conjugated at the 3′ terminus of the polynucleic acid molecule while the binding moiety is conjugated at the 5′ terminus of the polynucleic acid molecule. In some instances, the PEG moiety is conjugated to an internal site of the polynucleic acid molecule. In some instances, the PEG moiety, the binding moiety, or a combination thereof, are conjugated to an internal site of the polynucleic acid molecule. In some instances, the conjugation is a direct conjugation. In some instances, the conjugation is via native ligation.

In some aspects, the polyalkylene oxide (e.g., PEG) is a polydispers or monodispers compound. In some instances, polydispers material comprises disperse distribution of different molecular weight of the material, characterized by mean weight (weight average) size and dispersity. In some instances, the monodisperse PEG comprises one size of molecules. In some aspects, C is poly- or monodispersed polyalkylene oxide (e.g., PEG) and the indicated molecular weight represents an average of the molecular weight of the polyalkylene oxide, e.g., PEG, molecules.

In some aspects, the molecular weight of the polyalkylene oxide (e.g., PEG) is about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da.

In some aspects, C is polyalkylene oxide (e.g., PEG) and has a molecular weight of about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da. In some aspects, C is PEG and has a molecular weight of about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da. In some instances, the molecular weight of C is about 200 Da. In some instances, the molecular weight of C is about 300 Da. In some instances, the molecular weight of C is about 400 Da. In some instances, the molecular weight of C is about 500 Da. In some instances, the molecular weight of C is about 600 Da. In some instances, the molecular weight of C is about 700 Da. In some instances, the molecular weight of C is about 800 Da. In some instances, the molecular weight of C is about 900 Da. In some instances, the molecular weight of C is about 1000 Da. In some instances, the molecular weight of C is about 1100 Da. In some instances, the molecular weight of C is about 1200 Da. In some instances, the molecular weight of C is about 1300 Da. In some instances, the molecular weight of C is about 1400 Da. In some instances, the molecular weight of C is about 1450 Da. In some instances, the molecular weight of C is about 1500 Da. In some instances, the molecular weight of C is about 1600 Da. In some instances, the molecular weight of C is about 1700 Da. In some instances, the molecular weight of C is about 1800 Da. In some instances, the molecular weight of C is about 1900 Da. In some instances, the molecular weight of C is about 2000 Da. In some instances, the molecular weight of C is about 2100 Da. In some instances, the molecular weight of C is about 2200 Da. In some instances, the molecular weight of C is about 2300 Da. In some instances, the molecular weight of C is about 2400 Da. In some instances, the molecular weight of C is about 2500 Da. In some instances, the molecular weight of C is about 2600 Da. In some instances, the molecular weight of C is about 2700 Da. In some instances, the molecular weight of C is about 2800 Da. In some instances, the molecular weight of C is about 2900 Da. In some instances, the molecular weight of C is about 3000 Da. In some instances, the molecular weight of C is about 3250 Da. In some instances, the molecular weight of C is about 3350 Da. In some instances, the molecular weight of C is about 3500 Da. In some instances, the molecular weight of C is about 3750 Da. In some instances, the molecular weight of C is about 4000 Da. In some instances, the molecular weight of C is about 4250 Da. In some instances, the molecular weight of C is about 4500 Da. In some instances, the molecular weight of C is about 4600 Da. In some instances, the molecular weight of C is about 4750 Da. In some instances, the molecular weight of C is about 5000 Da. In some instances, the molecular weight of C is about 5500 Da. In some instances, the molecular weight of C is about 6000 Da. In some instances, the molecular weight of C is about 6500 Da. In some instances, the molecular weight of C is about 7000 Da. In some instances, the molecular weight of C is about 7500 Da. In some instances, the molecular weight of C is about 8000 Da. In some instances, the molecular weight of C is about 10,000 Da. In some instances, the molecular weight of C is about 12,000 Da. In some instances, the molecular weight of C is about 20,000 Da. In some instances, the molecular weight of C is about 35,000 Da. In some instances, the molecular weight of C is about 40,000 Da. In some instances, the molecular weight of C is about 50,000 Da. In some instances, the molecular weight of C is about 60,000 Da. In some instances, the molecular weight of C is about 100,000 Da.

In some aspects, the polyalkylene oxide (e.g., PEG) comprises discrete ethylene oxide units (e.g., four to about 48 ethylene oxide units). In some instances, the polyalkylene oxide comprising the discrete ethylene oxide units is a linear chain. In other cases, the polyalkylene oxide comprising the discrete ethylene oxide units is a branched chain.

In some instances, the polymer moiety C is a polyalkylene oxide (e.g., PEG) comprising discrete ethylene oxide units. In some cases, the polymer moiety C comprises between about 4 and about 48 ethylene oxide units. In some cases, the polymer moiety C comprises about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, or about 48 ethylene oxide units.

In some instances, the polymer moiety C is a discrete PEG comprising, e.g., between about 4 and about 48 ethylene oxide units. In some cases, the polymer moiety C is a discrete PEG comprising, e.g., about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, or about 48 ethylene oxide units. In some cases, the polymer moiety C is a discrete PEG comprising, e.g., about 4 ethylene oxide units. In some cases, the polymer moiety C is a discrete PEG comprising, e.g., about 5 ethylene oxide units. In some cases, the polymer moiety C is a discrete PEG comprising, e.g., about 6 ethylene oxide units. In some cases, the polymer moiety C is a discrete PEG comprising, e.g., about 7 ethylene oxide units. In some cases, the polymer moiety C is a discrete PEG comprising, e.g., about 8 ethylene oxide units. In some cases, the polymer moiety C is a discrete PEG comprising, e.g., about 9 ethylene oxide units. In some cases, the polymer moiety C is a discrete PEG comprising, e.g., about 10 ethylene oxide units. In some cases, the polymer moiety C is a discrete PEG comprising, e.g., about 11 ethylene oxide units. In some cases, the polymer moiety C is a discrete PEG comprising, e.g., about 12 ethylene oxide units. In some cases, the polymer moiety C is a discrete PEG comprising, e.g., about 13 ethylene oxide units. In some cases, the polymer moiety C is a discrete PEG comprising, e.g., about 14 ethylene oxide units. In some cases, the polymer moiety C is a discrete PEG comprising, e.g., about 15 ethylene oxide units. In some cases, the polymer moiety C is a discrete PEG comprising, e.g., about 16 ethylene oxide units. In some cases, the polymer moiety C is a discrete PEG comprising, e.g., about 17 ethylene oxide units. In some cases, the polymer moiety C is a discrete PEG comprising, e.g., about 18 ethylene oxide units. In some cases, the polymer moiety C is a discrete PEG comprising, e.g., about 19 ethylene oxide units. In some cases, the polymer moiety C is a discrete PEG comprising, e.g., about 20 ethylene oxide units. In some cases, the polymer moiety C is a discrete PEG comprising, e.g., about 21 ethylene oxide units. In some cases, the polymer moiety C is a discrete PEG comprising, e.g., about 22 ethylene oxide units. In some cases, the polymer moiety C is a discrete PEG comprising, e.g., about 23 ethylene oxide units. In some cases, the polymer moiety C is a discrete PEG comprising, e.g., about 24 ethylene oxide units. In some cases, the polymer moiety C is a discrete PEG comprising, e.g., about 25 ethylene oxide units. In some cases, the polymer moiety C is a discrete PEG comprising, e.g., about 26 ethylene oxide units. In some cases, the polymer moiety C is a discrete PEG comprising, e.g., about 27 ethylene oxide units. In some cases, the polymer moiety C is a discrete PEG comprising, e.g., about 28 ethylene oxide units. In some cases, the polymer moiety C is a discrete PEG comprising, e.g., about 29 ethylene oxide units. In some cases, the polymer moiety C is a discrete PEG comprising, e.g., about 30 ethylene oxide units. In some cases, the polymer moiety C is a discrete PEG comprising, e.g., about 31 ethylene oxide units. In some cases, the polymer moiety C is a discrete PEG comprising, e.g., about 32 ethylene oxide units. In some cases, the polymer moiety C is a discrete PEG comprising, e.g., about 33 ethylene oxide units. In some cases, the polymer moiety C is a discrete PEG comprising, e.g., about 34 ethylene oxide units. In some cases, the polymer moiety C is a discrete PEG comprising, e.g., about 35 ethylene oxide units. In some cases, the polymer moiety C is a discrete PEG comprising, e.g., about 36 ethylene oxide units. In some cases, the polymer moiety C is a discrete PEG comprising, e.g., about 37 ethylene oxide units. In some cases, the polymer moiety C is a discrete PEG comprising, e.g., about 38 ethylene oxide units. In some cases, the polymer moiety C is a discrete PEG comprising, e.g., about 39 ethylene oxide units. In some cases, the polymer moiety C is a discrete PEG comprising, e.g., about 40 ethylene oxide units. In some cases, the polymer moiety C is a discrete PEG comprising, e.g., about 41 ethylene oxide units. In some cases, the polymer moiety C is a discrete PEG comprising, e.g., about 42 ethylene oxide units. In some cases, the polymer moiety C is a discrete PEG comprising, e.g., about 43 ethylene oxide units. In some cases, the polymer moiety C is a discrete PEG comprising, e.g., about 44 ethylene oxide units. In some cases, the polymer moiety C is a discrete PEG comprising, e.g., about 45 ethylene oxide units. In some cases, the polymer moiety C is a discrete PEG comprising, e.g., about 46 ethylene oxide units. In some cases, the polymer moiety C is a discrete PEG comprising, e.g., about 47 ethylene oxide units. In some cases, the polymer moiety C is a discrete PEG comprising, e.g., about 48 ethylene oxide units.

In some cases, the polymer moiety C is dPEG® (Quanta Biodesign Ltd).

In some aspects, the polymer moiety C comprises a cationic mucic acid-based polymer (cMAP). In some instances, cMAP comprises one or more subunit of at least one repeating subunit, and the subunit structure is represented as Formula (V):

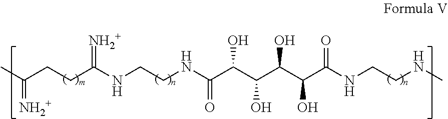

wherein m is independently at each occurrence 1, 2, 3, 4, 5, 6, 7, 8, 9, or 10, preferably 4-6 or 5; and n is independently at each occurrence 1, 2, 3, 4, or 5. In some aspects, m and n are, for example, about 10.

In some instances, cMAP is further conjugated to a PEG moiety, generating a cMAP-PEG copolymer, an mPEG-cMAP-PEGm triblock polymer, or a cMAP-PEG-cMAP triblock polymer.

In some instances, the PEG moiety is in a range of from about 500 Da to about 50,000 Da. In some instances, the PEG moiety is in a range of from about 500 Da to about 1000 Da, greater than 1000 Da to about 5000 Da, greater than 5000 Da to about 10,000 Da, greater than 10,000 to about 25,000 Da, greater than 25,000 Da to about 50,000 Da, or any combination of two or more of these ranges.

In some instances, the polymer moiety C is cMAP-PEG copolymer, an mPEG-cMAP-PEGm triblock polymer, or a cMAP-PEG-cMAP triblock polymer. In some cases, the polymer moiety C is cMAP-PEG copolymer. In other cases, the polymer moiety C is an mPEG-cMAP-PEGm triblock polymer. In additional cases, the polymer moiety C is a cMAP-PEG-cMAP triblock polymer.

In some aspects, the polymer moiety C is conjugated to the polynucleic acid molecule, the binding moiety, and optionally to the endosomolytic moiety as illustrated supra.

### Endosomolytic Moiety

In some aspects, a molecule of Formula (I): A-X-B-Y-C, further comprises an additional conjugating moiety. In some instances, the additional conjugating moiety is an endosomolytic moiety. In some cases, the endosomolytic moiety is a cellular compartmental release component, such as a compound capable of releasing from any of the cellular compartments known in the art, such as the endosome, lysosome, endoplasmic reticulum (ER), golgi apparatus, microtubule, peroxisome, or other vesicular bodies with the cell. In some cases, the endosomolytic moiety comprises an endosomolytic polypeptide, an endosomolytic polymer, an endosomolytic lipid, or an endosomolytic small molecule. In some cases, the endosomolytic moiety comprises an endosomolytic polypeptide. In other cases, the endosomolytic moiety comprises an endosomolytic polymer.

### Endosomolytic Polypeptides

In some aspects, a molecule of Formula (I): A-X-B-Y-C, is further conjugated with an endosomolytic polypeptide. In some cases, the endosomolytic polypeptide is a pH-dependent membrane active peptide. In some cases, the endosomolytic polypeptide is an amphipathic polypeptide. In additional cases, the endosomolytic polypeptide is a peptidomimetic. In some instances, the endosomolytic polypeptide comprises INF, melittin, meucin, or their respective derivatives thereof. In some instances, the endosomolytic polypeptide comprises INF or its derivatives thereof. In other cases, the endosomolytic polypeptide comprises melittin or its derivatives thereof. In additional cases, the endosomolytic polypeptide comprises meucin or its derivatives thereof.

In some instances, INF7 is a 24 residue polypeptide those sequence comprises CGIFGEIEELIEEGLENLIDWGNA (SEQ ID NO: 1), or GLFEAIEGFIENGWEGMIDGWYGC (SEQ ID NO: 2). In some instances, INF7 or its derivatives comprise a sequence of: GLFEAIEGFIENGWEGMIWDYGSGSCG (SEQ ID NO: 3), GLFEAIEGFIENGWEGMIDG WYG-(PEG)6-NH2 (SEQ ID NO: 4), or GLFEAIEGFIENGWEGMIWDYG-SGSC-K(GalNAc)2 (SEQ ID NO: 5).

In some cases, melittin is a 26 residue polypeptide those sequence comprises CLIGAILKVLATGLPTLISWIKNKRKQ (SEQ ID NO: 6), or GIGAVLKVLTTGLPALISWIKRKRQQ (SEQ ID NO: 7). In some instances, melittin comprises a polypeptide sequence as described in U.S. Pat. No. 8,501,930.

In some instances, meucin is an antimicrobial peptide (AMP) derived from the venom gland of the scorpion Mesobuthus eupeus. In some instances, meucin comprises of meucin-13 those sequence comprises IFGAIAGLLKNIF-NH2 (SEQ ID NO: 8) and meucin-18 those sequence comprises FFGHLFKLATKIIPSLFQ (SEQ ID NO: 9).

In some instances, the endosomolytic polypeptide comprises a polypeptide in which its sequence is at least 50%, 60%, 70%, 80%, 90%, 95%, or 99% sequence identity to INF7 or its derivatives thereof, melittin or its derivatives thereof, or meucin or its derivatives thereof. In some instances, the endosomolytic moiety comprises INF7 or its derivatives thereof, melittin or its derivatives thereof, or meucin or its derivatives thereof.

In some instances, the endosomolytic moiety is INF7 or its derivatives thereof. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NOs: 1-5. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 1. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 2-5. In some cases, the endosomolytic moiety comprises SEQ ID NO: 1. In some cases, the endosomolytic moiety comprises SEQ ID NO: 2-5.

In some cases, the endosomolytic moiety consists of SEQ ID NO: 1. In some cases, the endosomolytic moiety consists of SEQ ID NO: 2-5.

In some instances, the endosomolytic moiety is melittin or its derivatives thereof. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NOs: 6 or 7. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 6. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 7. In some cases, the endosomolytic moiety comprises SEQ ID NO: 6. In some cases, the endosomolytic moiety comprises SEQ ID NO: 7. In some cases, the endosomolytic moiety consists of SEQ ID NO: 6. In some cases, the endosomolytic moiety consists of SEQ ID NO: 7.

In some instances, the endosomolytic moiety is meucin or its derivatives thereof. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NOs: 8 or 9. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 8. In some cases, the endosomolytic moiety comprises a polypeptide having at least 50%, 55%, 60%, 65%, 70%, 75%, 80%, 85%, 90%, 95%, 96%, 97%, 98%, 99%, or 100% sequence identity to SEQ ID NO: 9. In some cases, the endosomolytic moiety comprises SEQ ID NO: 8. In some cases, the endosomolytic moiety comprises SEQ ID NO: 9. In some cases, the endosomolytic moiety consists of SEQ ID NO: 8. In some cases, the endosomolytic moiety consists of SEQ ID NO: 9.

In some instances, the endosomolytic moiety comprises a sequence as illustrated in Table 1.

TABLE 1
Exemplary endosomolytic moiety

| | | | SEQ | |
|---|---|---|---|---|
| | | | ID | |
| Name | Origin | Amino Acid Sequence | NO: | Type |
| Pep-1 | NLS from Simian Virus | KETWWETWWTEWSQPKKKRKV | 10 | Primary |
| | 40 large antigen and | | | amphipathic |
| | Reverse transcriptase of | | | |
| | HIV | | | |
| pVEC | VE-cadherin | LLIILRRRRIRKQAHAHSK | 11 | Primary |
| | | | | amphipathic |
| VT5 | Synthetic peptide | DPKGDPKGVTVTVTVTVTGKGDP | 12 | β-sheet |
| | | KPD | | amphipathic |
| C105Y | 1-antitrypsin | CSIPPEVKFNKPFVYLI | 13 | — |
| Transportan | Galanin and mastoparan | GWTLNSAGYLLGKINLKALAALA | 14 | Primary |
| | | KKIL | | amphipathic |
| TP10 | Galanin and mastoparan | AGYLLGKINLKALAALAKKIL | 15 | Primary |
| | | | | amphipathic |
| MPG | A hydrofobic domain | GALFLGFLGAAGSTMGA | 16 | β-sheet |
| | from the fusion | | | amphipathic |
| | sequence of HIV gp41 | | | |
| | and NLS of SV40 T | | | |
| | antigen | | | |
| gH625 | Glycoprotein gH of | HGLASTLTRWAHYNALIRAF | 17 | Secondary |
| | HSV type I | | | amphipathic |
| | | | | α-helical |
| CADY | PPTG1 peptide | GLWRALWRLLRSLWRLLWRA | 18 | Secondary |
| | | | | amphipathic |
| | | | | α-helical |
| GALA | Synthetic peptide | WEAALAEALAEALAEHLAEALAE | 19 | Secondary |
| | | ALEALAA | | amphipathic |
| | | | | α-helical |
| INF | Influenza HA2 fusion | GLFEAIEGFIENGWEGMIDGWYGC | 20 | Secondary |
| | peptide | | | amphipathic |
| | | | | α-helical/ |
| | | | | pH- |
| | | | | dependent |
| | | | | membrane |
| | | | | active |
| | | | | peptide |
| HA2E5- | Influenza HA2 subunit | GLFGAIAGFIENGWEGMIDGWYG | 21 | Secondary |
| TAT | of influenza virus X31 | | | amphipathic |
| | strain fusion peptide | | | α-helical/ |
| | | | | pH- |
| | | | | dependent |
| | | | | membrane |
| | | | | active |
| | | | | peptide |
| HA2- | Influenza HA2 subunit | GLFGAIAGFIENGWEGMIDGRQIKI | 22 | pH- |
| penetratin | of influenza virus X31 | WFQNRRMKW | | dependent |
| | strain fusion peptide | KK-amide | | membrane |
| | | | | active |
| | | | | peptide |
| HA-K4 | Influenza HA2 subunit | GLFGAIAGFIENGWEGMIDG- | 23 | pH- |
| | of influenza virus X31 | SSKKKK | | dependent |
| | strain fusion peptide | | | membrane |
| | | | | active |
| | | | | peptide |
| HA2E4 | Influenza HA2 subunit | GLFEAIAGFIENGWEGMIDGGGYC | 24 | pH- |
| | of influenza virus X31 | | | dependent |
| | strain fusion peptide | | | membrane |
| | | | | active |
| | | | | peptide |
| H5WYG | HA2 analogue | GLFHAIAHFIHGGWH | 25 | pH- |
| | | GLIHGWYG | | dependent |
| | | | | membrane |
| | | | | active |
| | | | | peptide |
| GALA- | INF3 fusion peptide | GLFEAIEGFIENGWEGLAEALAEAL | 26 | pH- |
| INF3- | | EALAA- | | dependent |
| (PEG)6-NH | | (PEG)6-NH2 | | membrane |
| | | | | active |
| | | | | peptide |
| CM18- | Cecropin-A-Melittin₂₋₁₂ | KWKLFKKIGAVLKVLTTG- | 27 | pH- |
| TAT11 | (CM₁₈) fusion peptide | YGRKKRRQRRR | | dependent |
| | | | | membrane |
| | | | | active |
| | | | | peptide |

In some cases, the endosomolytic moiety comprises a Bak BH3 polypeptide which induces apoptosis through antagonization of suppressor targets such as Bcl-2 and/or Bcl-xL. In some instances, the endosomolytic moiety comprises a Bak BH3 polypeptide described in Albarran, et al., “Efficient intracellular delivery of a pro-apoptotic peptide with a pH-responsive carrier,” Reactive & Functional Polymers 71: 261-265 (2011).

In some instances, the endosomolytic moiety comprises a polypeptide (e.g., a membrane penetrating polypeptide) as described in PCT Publication Nos. WO2013/166155 or WO2015/069587.

### Membrane Penetrating Peptide

The terms “membrane penetrating peptide” and “MPP” are used interchangeably and refer to cationic cell penetrating peptides, also called transport peptides, carrier peptides, or peptide transduction domains. The peptides, provided herein, have the capability of inducing membrane penetration within 100% of cells of a given cell culture population and allow macromolecular translocation within multiple tissues in vivo upon systemic administration. In various aspects, a MPP embodiment of the disclosure may include an arginine-rich peptide as described further below.

Provided herein are oligonucleotides chemically linked to a membrane penetrating peptide. The membrane penetrating peptide enhances activity, cellular distribution, or cellular uptake of the oligonucleotide. In particular, the cell-penetrating peptide is a linear, or non-cyclic, peptide. In some aspects, the MPP can be an arginine-rich peptide. The oligonucleotides can additionally be chemically-linked to one or more heteroalkyl moieties (e.g., polyethylene glycol) that further enhance the activity, cellular distribution, or cellular uptake of the oligonucleotide. In one exemplary embodiment, the polypeptide, e.g., the arginine-rich polypeptide, is covalently coupled at its N-terminal or C-terminal residue to either end, or both ends, of the oligonucleotide.

In some instances, the membrane penetrating peptide comprises a sequence as illustrated in Table 2.

TABLE 2
Exemplary membrane penetrating peptide

| | | | | SEQ |
|---|---|---|---|---|
| | | Theoretical | | ID |
| CPP Name | CPP Class | Net Charge | Amino Acid Sequence | NO: |
| Arginine-12 | Cationic | 12 | RRRRRRRRRRRR | 1000 |
| MPG | Amphipathic | 5 | GLAFLGFLGAAGSTMGAWSQPKKKRKV | 1001 |
| | (cationic I) | | | |
| Bac7 | Proline rich | 9 | RRIRPRPPRLPRPRPRPLPFPRPG | 1002 |
| TAT | Cationic | 8 | RKKRRQRRR | 1003 |
| Arginine-10 | Cationic | 10 | RRRRRRRRRR | 1004 |
| DPV6 | Cationic | 9 | GRPRESGKKRKRKRLKP | 1005 |
| S413-PVrev | Amphipathic | 9 | ALWKTLLKKVLKAPKKKRKV | 1006 |
| HRSV | Cationic | 6 | RRIPNRRPRR | 1007 |
| HTLV-II | Cationic | 8 | TRRQRTRRARRNR | 1008 |
| Rex | | | | |
| L-2 | Amphipathic | 9 | HARIKPTFRRLKWKYKGKFW | 1009 |
| Melittin | Amphipathic | 5 | GIGAVLKVLTTGLPALISWIKRKRQQ | 1010 |
| DPV15 | Cationic | 6 | LRRERQSRLRRERQSR | 1011 |
| Arginine-9 | Cationic | 9 | RRRRRRRRR | 1012 |
| Penetratin | Cationic | 7 | RQIKIWFQNRRMKWKK | 1013 |
| Yeast GCN4 | Cationic | 9 | KRARNTEAARRSRARKLQRMKQ | 1014 |
| PDX-1 | Cationic | 8 | RHIKIWFQNRRMKWKK | 1015 |
| Arginine-8 | Cationic | 8 | RRRRRRRR | 1016 |
| BMV Gag | Cationic | 8 | KMTRAQRRAAARRNRWTAR | 1017 |
| SynB1 | Amphipathic | 6 | RGGRLSYSRRRFSTSTGR | 1018 |
| Knotted-1 | Cationic | 6 | KQINNWFINQRKRHWK | 1019 |
| IVV-14 | Hydrophobic | 4 | KLWMRWYSPTTRRYG | 1020 |
| W/R | Amphipathic | 6 | RRWWRRWRR | 1021 |
| | (cationic II) | | | |
| Engrailed-2 | Cationic | 6 | SQIKIWFQNKRAKIKK | 1022 |
| DPV15b | Cationic | 7 | GAYDLRRRERQSRLRRRERQSR | 1023 |
| Yeast PrP6 | Cationic | 6 | TRRNKRNRIQEQLNRK | 1024 |
| DPV7 | Cationic | 8 | GKRKKKGKLGKKRDP | 1025 |
| HoxA-13 | Cationic | 5 | RQVTIWFQNRRVKEKK | 1026 |
| AIP6 | Amphipathic | 3 | RLRWR | 1027 |
| | (proline rich) | | | |
| (PPR)5 | Cationic | 5 | PPRPPRPPRPPRPPR | 1028 |
| CAYH | — | 4 | CAYHRLRRC | 1029 |
| DPV10 | Cationic | 6 | SRRARRSPRHLGSG | 1030 |
| (PPR)4 | Amphipathic | 4 | PPRPPRPPRPPR | 1031 |
| | (proline rich) | | | |
| P22 N | Cationic | 7 | NAKTRRHERRRKLAIER | 1032 |
| DPV1047 | Cationic | 7 | VKRGLKLRHVRPRVTRMDV | 1033 |
| SVM4 | Amphipathic | 7 | LYKKGPAKKG RPPLRGWFH | 1034 |
| | (cationic) | | | |
| .phi.21N | Cationic | 5 | TAKTRYKARRAELIAERR | 1035 |
| (12-29) | | | | |
| SVM3 | Amphipathic | 6 | KGTYKKKL MRIPLKGT | 1036 |
| | (cationic) | | | |
| (PPR)3 | Amphipathic | 3 | PPRPPRPPR | 1037 |
| | (proline rich) | | | |
| SVM2 | — | 5 | RASKRDGSWVKKLHRILE | 1038 |
| Buforin 2 | Amphipathic | 7 | TRSSRAGLQWPVGRVHRLLRK | 1039 |
| SVM1 | — | 6 | FKIYDKKVRTRVVKH | 1040 |
| SAP | Amphipathic | 3 | VRLPPPVRLPPPVRLPPP | 1041 |
| | (proline rich) | | | |
| 435b | Hydrophobic | 1 | GPFHFYQFLFPPV | 1042 |
| Pept1 | Hydrophobic | 2 | PLILLRLLRGQF | 1043 |
| YTA2 | — | 5 | YTAIAWVKAFIRKLRK | 1044 |
| Pep-1 | Amphipathic | 2 | KETWWETWWTEWSQPKKRKV | 1045 |
| | (cationic I) | | | |
| EB-1 | Amphipathic | 9 | LIRLWSHLIHIWFQNRRLKWKKK | 1046 |
| | (cationic II) | | | |
| Pyrrho- | Proline rich | 3 | VDKGSYLPRPTPPRPIYNRN | 1047 |
| coricin | | | | |
| lamda.N | Cationic | 4 | MDAQTRRRERRAEKQAQWKAAN | 1048 |
| (1-22) | | | | |
| 439a | Hydrophobic | 3 | GSPWGLQHHPPRT | 1049 |
| MAP | Amphipathic | 5 | KLALKALKALKAALKLA | 1050 |
| | (cationic II) | | | |
| Bip | Hydrophobic | 1 | IPALK | 1051 |
| Bip | Hydrophobic | 1 | VPALR | 1052 |
| pVEC | Amphipathic | 8 | LLIILRRRIRKQAHAHSK | 1053 |
| | (I) | | | |
| YTA4 | — | 5 | IAWVKAFIRKL RKGPLG | 1054 |
| K-FGF + | Amphipathic | 4 | AAVLLPVLLAAPVQRKRQKLP | 1055 |
| NLS | | | | |
| HN-1 | Hydrophobic | 2 | TSPLNIHNGQKL | 1056 |
| Bip | Hydrophobic | 1 | VPTLK | 1057 |
| Bip | Hydrophobic | 1 | VSALK | 1058 |
| VT5 | Amphipathic | 0 | DPKGDPKGVTVTVTVTVTGKGDPKPD | 1059 |
| | (beta sheet) | | | |
| Trans- | Amphipathic | 4 | AGYLLGKINLKALAALAKKIL | 1060 |
| portan 10 | (cationic II) | | | |
| SAP(E) | Amphipathic | −3 | VELPPPVELPPPVELPPP | 1061 |
| CADY | Amphipathic | 5 | GLWRALWRLLRSLWRLLWRA | 1062 |
| | (cationic II) | | | |
| PreS2- TLM | Amphipathic | 0 | PLSSIFSRIGDP | 1063 |

The efficient intracellular delivery of many pharmaceutically active compounds, such as proteins and nucleic acids, is an outstanding challenge in the field of drug delivery. Many large macromolecules are unable to cross the plasma membrane, and often end up trapped in endosomes and degraded in lysosomes. Over the past few decades, multiple approaches have been developed to promote the cytosolic delivery of large macromolecules, including supercharging the molecules with a high density of charge, complexing the molecules with delivery vehicles such as liposomes or nanoparticles, and conjugating to membrane penetrating peptides.

Since the discovery that a twenty amino acid fragment of the trans-activating transcriptional activator (TAT) from HIV-1 enabled a protein to cross the plasma membrane, hundreds of membrane penetrating peptides (MPPs) have been reported to improve entry into the cytoplasm. These peptides have been derived from many natural sources, such as viral proteins, DNA-binding proteins, signal peptides, and antimicrobial peptides. Additionally, MPPs have been rationally designed and identified from DNA-encoded peptide libraries. MPP sequences exhibit a wide diversity of physicochemical properties and range from highly cationic to amphipathic to hydrophobic. While these experiments provide evidence of cell penetration, they fail to address the question of whether the MPP is suitable for the delivery of a particular macromolecular cargo. In spite of that, MPPs have been utilized to improve the cellular delivery of peptides, enzymes, antibodies, oligonucleotides, nanoparticles, and chemotherapeutics.

### Antibody-Peptide-PMO Conjugate (PPMO-AOC)

One application of MPPs is for the delivery of phosphorodiamidate morpholino oligonucleotides (PMO). PMOs are a charge-neutral antisense therapeutic in which the ribose sugar is replaced with a methylenemorpholine ring and the phosphodiester backbone is replaced with a phosphorodiamidate backbone. PMOs bind to pre-mRNA and can alter gene splicing through a process known as “exon-skipping.” Recently, the PMO Eteplirsen became the first and only FDA-approved therapy to treat the underlying genetic cause of Duchenne muscular dystrophy (DMD) by skipping exon 51 of the dystrophin gene. Although PMO therapies such as Eteplirsen show significant promise, the dosages required are often multiple grams per week due to limited intracellular delivery. Creating conjugates between MPPs and PMOs has been one effective approach in improving delivery. O'Donovan et al. have looked at a modest library of sixteen different MPP-PMO conjugates and Moulton et al. have identified arginine-rich peptides that have improved the delivery of PMO cargoes for DMD. However, there has yet to be a systematic investigation of the features of MPPs that promote PMO delivery.

Provided herein are antibody-peptide-oligonucleotide conjugates comprising an antibody conjugated to an oligonucleotide covalently bound to a membrane penetrating peptide (MPP) or a MPP covalently linked to an oligonucleotide. Also provided herein are methods of treating a disease in a subject in need thereof, comprising administering to the subject an antibody-peptide-oligonucleotide conjugate described herein.

Provided herein are antibody-peptide-oligonucleotide conjugate comprising an oligonucleotide covalently bound to a membrane penetrating peptide. Also provided herein are methods of treating a disease in a subject in need thereof, comprising administering to the subject an antibody-peptide-oligonucleotide conjugate described herein. The oligonucleotides, and thereby the antibody-peptide-oligonucleotide conjugates, described herein display stronger affinity for DNA and RNA without compromising sequence selectivity, relative to native or unmodified oligonucleotides. In some aspects, the oligonucleotides of the disclosure minimize or prevent cleavage by RNase H. In some aspects, the antisense oligonucleotides of the disclosure do not activate RNase H.

The peptides described herein impart to their corresponding antibody-peptide-oligonucleotide conjugates lower toxicity, enhance the activity of the oligonucleotide, improve pharmacokinetics and tissue distribution, improve cellular delivery, and impart both reliable and controllable in vivo distribution.

Membrane penetrating peptides can facilitate the intracellular delivery of large therapeutically relevant molecules, including proteins and oligonucleotides. Although hundreds of MPP sequences are described in the literature derived both from nature and from rational design, the performance of any given sequence relies on it being well-matched to the cargo of interest. The present experiments focus specifically on antibody-MPPs for the delivery of phosphorodiamidate morpholino oligonucleotides (PMOs), a compelling type of antisense therapeutic that has recently been FDA approved for the treatment of Duchenne Muscular Dystrophy. Wide discrepancies in the performance of known MPPs for delivery of PMO cargo as opposed to a fluorophore cargo have been described herein. Therefore, the use of computational methods to predict which peptide sequences would perform best specifically for PMO delivery would be an efficient method for determining active antibody MPP-PMO conjugates.

### Linkers

In some aspects, a linker described herein is a cleavable linker or a non-cleavable linker. In some instances, the linker is a cleavable linker. In other instances, the linker is a non-cleavable linker.

In some cases, the linker is a non-polymeric linker. A non-polymeric linker refers to a linker that does not contain a repeating unit of monomers generated by a polymerization process. Exemplary non-polymeric linkers include, but are not limited to, C₁-C₆ alkyl group (e.g., a C5, C4, C3, C2, or C1 alkyl group), homobifunctional cross linkers, heterobifunctional cross linkers, peptide linkers, traceless linkers, self-immolative linkers, maleimide-based linkers, or combinations thereof. In some cases, the non-polymeric linker comprises a C₁-C₆ alkyl group (e.g., a C5, C4, C3, C2, or C1 alkyl group), a homobifunctional cross linker, a heterobifunctional cross linker, a peptide linker, a traceless linker, a self-immolative linker, a maleimide-based linker, or a combination thereof. In additional cases, the non-polymeric linker does not comprise more than two of the same type of linkers, e.g., more than two homobifunctional cross linkers, or more than two peptide linkers. In further cases, the non-polymeric linker optionally comprises one or more reactive functional groups.

In some instances, the non-polymeric linker does not encompass a polymer that is described above. In some instances, the non-polymeric linker does not encompass a polymer encompassed by the polymer moiety C. In some cases, the non-polymeric linker does not encompass a polyalkylene oxide (e.g., PEG). In some cases, the non-polymeric linker does not encompass a PEG.

In some instances, the linker comprises a homobifunctional linker. Exemplary homobifunctional linkers include, but are not limited to, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[D-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide).

In some aspects, the linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio)toluene (sMPT), sulfosuccinimidyl-6-[α-methyl-α-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(γ-maleimidobutyryloxy)succinimide ester (GMBs), N-(γ-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M2C2H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(ρ-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(ρ-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), p-nitrophenyl diazopyruvate (pNPDP), p-nitrophenyl-2-diazo-3,3,3-trifluoropropionate (PNP-DTP), sulfhydryl-reactive and photoreactive cross-linkers such asl-(ρ-Azidosalicylamido)-4-(iodoacetamido)butane (AsIB), N-[4-(ρ-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as ρ-azidobenzoyl hydrazide (ABH), carboxylate-reactive and photoreactive cross-linkers such as 4-(ρ-azidosalicylamido)butylamine (AsBA), and arginine-reactive and photoreactive cross-linkers such as ρ-azidophenyl glyoxal (APG).

In some instances, the linker comprises a reactive functional group. In some cases, the reactive functional group comprises a nucleophilic group that is reactive to an electrophilic group present on a binding moiety. Exemplary electrophilic groups include carbonyl groups-such as aldehyde, ketone, carboxylic acid, ester, amide, enone, acyl halide or acid anhydride. In some aspects, the reactive functional group is aldehyde. Exemplary nucleophilic groups include hydrazide, oxime, amino, hydrazine, thiosemicarbazone, hydrazine carboxylate, and arylhydrazide.

In some aspects, the linker comprises a maleimide group. In some instances, the maleimide group is also referred to as a maleimide spacer. In some instances, the maleimide group further encompasses a caproic acid, forming maleimidocaproyl (mc). In some cases, the linker comprises maleimidocaproyl (mc). In some cases, the linker is maleimidocaproyl (mc). In other instances, the maleimide group comprises a maleimidomethyl group, such as succinimidyl-4-(N-maleimidomethyl)cyclohexane-1-carboxylate (sMCC) or sulfosuccinimidyl-4-(N-maleimidomethyl)cyclohexane-1-carboxylate (sulfo-sMCC) described above.

In some aspects, the maleimide group is a self-stabilizing maleimide. In some instances, the self-stabilizing maleimide utilizes diaminopropionic acid (DPR) to incorporate a basic amino group adjacent to the maleimide to provide intramolecular catalysis of tiosuccinimide ring hydrolysis, thereby eliminating maleimide from undergoing an elimination reaction through a retro-Michael reaction. In some instances, the self-stabilizing maleimide is a maleimide group described in Lyon, et al., “Self-hydrolyzing maleimides improve the stability and pharmacological properties of antibody-drug conjugates,” Nat. Biotechnol. 32(10):1059-1062 (2014). In some instances, the linker comprises a self-stabilizing maleimide. In some instances, the linker is a self-stabilizing maleimide.

In some aspects, the linker comprises a peptide moiety. In some instances, the peptide moiety comprises at least 2, 3, 4, 5, 6, 7, 8, or more amino acid residues. In some instances, the peptide moiety is a cleavable peptide moiety (e.g., either enzymatically or chemically). In some instances, the peptide moiety is a non-cleavable peptide moiety. In some instances, the peptide moiety comprises Val-Cit (valine-citrulline), Gly-Gly-Phe-Gly (SEQ ID NO: 973), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 974), or Gly-Phe-Leu-Gly (SEQ ID NO: 975). In some instances, the linker comprises a peptide moiety such as: Val-Cit (valine-citrulline), Gly-Gly-Phe-Gly (SEQ ID NO: 973), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 974), or Gly-Phe-Leu-Gly (SEQ ID NO: 975). In some cases, the linker comprises Val-Cit. In some cases, the linker is Val-Cit.

In some aspects, the linker comprises a benzoic acid group, or its derivatives thereof. In some instances, the benzoic acid group or its derivatives thereof comprise paraaminobenzoic acid (PABA). In some instances, the benzoic acid group or its derivatives thereof comprise gamma-aminobutyric acid (GABA).

In some aspects, the linker comprises one or more of a maleimide group, a peptide moiety, and/or a benzoic acid group, in any combination. In some aspects, the linker comprises a combination of a maleimide group, a peptide moiety, and/or a benzoic acid group. In some instances, the maleimide group is maleimidocaproyl (mc). In some instances, the peptide group is val-cit. In some instances, the benzoic acid group is PABA. In some instances, the linker comprises a mc-val-cit group. In some cases, the linker comprises a val-ci2′-PABA group. In additional cases, the linker comprises a mc-val-ci2′-PABA group.

In some aspects, the linker is a self-immolative linker or a self-elimination linker. In some cases, the linker is a self-immolative linker. In other cases, the linker is a self-elimination linker (e.g., a cyclization self-elimination linker). In some instances, the linker comprises a linker described in U.S. Pat. No. 9,089,614 or PCT Publication No. WO2015038426.

In some aspects, the linker is a dendritic type linker. In some instances, the dendritic type linker comprises a branching, multifunctional linker moiety. In some instances, the dendritic type linker is used to increase the molar ratio of polynucleotide B to the binding moiety A. In some instances, the dendritic type linker comprises PAMAM dendrimers.

In some aspects, the linker is a traceless linker or a linker in which after cleavage does not leave behind a linker moiety (e.g., an atom or a linker group) to a binding moiety A, a polynucleotide B, a polymer C, or an endosomolytic moiety D. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some cases, the linker is a traceless aryl-triazene linker as described in Hejesen, et al., “A traceless aryl-triazene linker for DNA-directed chemistry,” Org Biomol Chem 11(15): 2493-2497 (2013). In some instances, the linker is a traceless linker described in Blaney, et al., “Traceless solid-phase organic synthesis,” Chem. Rev. 102: 2607-2024 (2002). In some instances, a linker is a traceless linker as described in U.S. Pat. No. 6,821,783.

In some instances, the linker is a linker described in U.S. Pat. Nos. 6,884,869; 7,498,298; 8,288,352; 8,609,105; or 8,697,688; U.S. Patent Publication Nos. 2014/0127239; 2013/028919; 2014/286970; 2013/0309256; 2015/037360; or 2014/0294851; or PCT Publication Nos. WO2015057699; WO2014080251; WO2014197854; WO2014145090; or WO2014177042.

In some aspects, X, Y, and L are independently a bond or a linker. In some instances, X, Y, and L are independently a bond. In some cases, X, Y, and L are independently a linker.

In some instances, X is a bond or a linker, e.g., a non-polymeric linker. In some instances, X is a bond. In some instances, X is a non-polymeric linker. In some instances, the non-polymeric linker is a C₁-C₆ alkyl group. In some cases, X is a C₁-C₆ alkyl group, such as for example, a C5, C4, C3, C2, or C1 alkyl group. In some cases, the C₁-C₆ alkyl group is an unsubstituted C₁-C₆ alkyl group. As used in the context of a non-polymeric linker, and in particular in the context of X, alkyl means a saturated straight or branched hydrocarbon radical containing up to six carbon atoms. In some instances, X includes a homobifunctional linker or a heterobifunctional linker described supra. In some cases, X includes a heterobifunctional linker. In some cases, X includes sMCC. In other instances, X includes a heterobifunctional linker optionally conjugated to a C1-C6 alkyl group. In other instances, X includes sMCC optionally conjugated to a C1-C6 alkyl group. In additional instances, X does not encompass a polymer encompassed by the polymer moiety C, e.g., X does not encompass a polyalkylene oxide (e.g., a PEG molecule).

In some instances, Y is a bond or a linker, e.g., a non-polymeric linker. In some instances, Y is a bond. In other cases, Y is a non-polymeric linker. In some aspects, Y is a C₁-C₆ alkyl group. In some instances, Y is a homobifunctional linker or a heterobifunctional linker described supra. In some instances, Y is a homobifunctional linker described supra. In some instances, Y is a heterobifunctional linker described supra. In some instances, Y comprises a maleimide group, such as maleimidocaproyl (mc) or a self-stabilizing maleimide group described above. In some instances, Y comprises a peptide moiety, such as Val-Cit. In some instances, Y comprises a benzoic acid group, such as PABA. In additional instances, Y comprises a combination of a maleimide group, a peptide moiety, and/or a benzoic acid group. In additional instances, Y comprises a me group. In additional instances, Y comprises a mc-val-cit group. In additional instances, Y comprises a val-cit-PABA group. In additional instances, Y comprises a mc-val-cit-PABA group. In some cases, Y does not encompass a polymer encompassed by the polymer moiety C, e.g., Y does not encompass a polyalkylene oxide (e.g., a PEG molecule).

In some instances, L is a bond or a linker, optionally a non-polymeric linker. In some cases, L is a bond. In other cases, L is a linker, optionally a non-polymeric linker. In some aspects, L is a C1-C6 alkyl group. In some instances, L is a homobifunctional linker or a heterobifunctional linker described supra. In some instances, L is a homobifunctional linker described supra. In some instances, L is a heterobifunctional linker described supra. In some instances, L comprises a maleimide group, such as maleimidocaproyl (mc) or a self-stabilizing maleimide group described above. In some instances, L comprises a peptide moiety, such as Val-Cit. In some instances, L comprises a benzoic acid group, such as PABA. In additional instances, L comprises a combination of a maleimide group, a peptide moiety, and/or a benzoic acid group.

In additional instances, L comprises a me group. In additional instances, L comprises a me-val-cit group. In additional instances, L comprises a val-ci2′-PABA group. In additional instances, L comprises a me-val-ci2′-PABA group. In some cases, L, when optionally as a non-polymeric linker, does not encompass a polymer encompassed by the polymer moiety C, e.g., Y does not encompass a polyalkylene oxide (e.g., a PEG molecule).

### Pharmaceutical Formulation

In some aspects, the pharmaceutical formulations described herein are administered to a subject by multiple administration routes, including but not limited to, parenteral (e.g., intravenous, subcutaneous, intramuscular), oral, intranasal, buccal, rectal, or transdermal administration routes. In some instances, the pharmaceutical composition describe herein is formulated for parenteral (e.g., intravenous, subcutaneous, intramuscular, intra-arterial, intraperitoneal, intrathecal, intracerebral, intracerebroventricular, or intracranial) administration. In other instances, the pharmaceutical composition describe herein is formulated for oral administration. In still other instances, the pharmaceutical composition describe herein is formulated for intranasal administration.

In some aspects, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some instances, the pharmaceutical formulation includes multiparticulate formulations. In some instances, the pharmaceutical formulation includes nanoparticle formulations. In some instances, nanoparticles comprise cMAP, cyclodextrin, or lipids. In some cases, nanoparticles comprise solid lipid nanoparticles, polymeric nanoparticles, self-emulsifying nanoparticles, liposomes, microemulsions, or micellar solutions. Additional exemplary nanoparticles include, but are not limited to, paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes and quantum dots. In some instances, a nanoparticle is a metal nanoparticle, e.g., a nanoparticle of scandium, titanium, vanadium, chromium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum, gold, gadolinium, aluminum, gallium, indium, tin, thallium, lead, bismuth, magnesium, calcium, strontium, barium, lithium, sodium, potassium, boron, silicon, phosphorus, germanium, arsenic, antimony, and combinations, alloys or oxides thereof.

In some instances, a nanoparticle includes a core or a core and a shell, as in a core-shell nanoparticle.

In some instances, a nanoparticle is further coated with molecules for attachment of functional elements (e.g., with one or more of a polynucleic acid molecule or binding moiety described herein). In some instances, a coating comprises chondroitin sulfate, dextran sulfate, carboxymethyl dextran, alginic acid, pectin, carragheenan, fucoidan, agaropectin, porphyran, karaya gum, gellan gum, xanthan gum, hyaluronic acids, glucosamine, galactosamine, chitin (or chitosan), polyglutamic acid, polyaspartic acid, lysozyme, cytochrome C, ribonuclease, trypsinogen, chymotrypsinogen, α-chymotrypsin, polylysine, polyarginine, histone, protamine, ovalbumin or dextrin or cyclodextrin. In some instances, a nanoparticle comprises a graphene-coated nanoparticle.

In some cases, a nanoparticle has at least one dimension of less than about 500 nm, 400 nm, 300 nm, 200 nm, or 100 nm.

In some instances, the nanoparticle formulation comprises paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes or quantum dots. In some instances, a polynucleic acid molecule or a binding moiety described herein is conjugated either directly or indirectly to the nanoparticle. In some instances, at least 1, 5, 10, 15, 20, 30, 40, 50, 60, 70, 80, 90, 100 or more polynucleic acid molecules or binding moieties described herein are conjugated either directly or indirectly to a nanoparticle.

In some aspects, the pharmaceutical formulation comprise a delivery vector, e.g., a recombinant vector, the delivery of the polynucleic acid molecule into cells. In some instances, the recombinant vector is DNA plasmid. In other instances, the recombinant vector is a viral vector. Exemplary viral vectors include vectors derived from adeno-associated virus, retrovirus, adenovirus, or alphavirus. In some instances, the recombinant vectors capable of expressing the polynucleic acid molecules provide stable expression in target cells. In additional instances, viral vectors are used that provide for transient expression of polynucleic acid molecules.

In some aspects, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999).

In some instances, the pharmaceutical formulations further include pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some instances, the pharmaceutical formulations further include diluent which are used to stabilize compounds because they provide a more stable environment. Salts dissolved in buffered solutions (which also provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol has a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Therapeutic Regimens

In some aspects, the pharmaceutical compositions described herein are administered for therapeutic applications. In some aspects, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In some aspects, one or more pharmaceutical compositions are administered simultaneously, sequentially, or at an interval period of time. In some aspects, one or more pharmaceutical compositions are administered simultaneously. In some cases, one or more pharmaceutical compositions are administered sequentially. In additional cases, one or more pharmaceutical compositions are administered at an interval period of time (e.g., the first administration of a first pharmaceutical composition is on day one followed by an interval of at least 1, 2, 3, 4, 5, or more days prior to the administration of at least a second pharmaceutical composition).

In some aspects, two or more different pharmaceutical compositions are coadministered. In some instances, the two or more different pharmaceutical compositions are coadministered simultaneously. In some cases, the two or more different pharmaceutical compositions are coadministered sequentially without a gap of time between administrations. In other cases, the two or more different pharmaceutical compositions are coadministered sequentially with a gap of about 0.5 hour, 1 hour, 2 hour, 3 hour, 12 hours, 1 day, 2 days, or more between administrations.

In the case wherein the patient's status does improve, upon the doctor's discretion the administration of the composition is given continuously; alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained.

In some aspects, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages is altered depending on a number of variables, not limited to the activity of the compound used, the disease or condition to be treated, the mode of administration, the requirements of the individual subject, the severity of the disease or condition being treated, and the judgment of the practitioner.

In some aspects, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred.

The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Kits/Article of Manufacture

Disclosed herein, in certain aspects, are kits and articles of manufacture for use with one or more of the compositions and methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include target nucleic acid molecule described herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain aspects, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some aspects, the mammal is a human. In some aspects, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein the terms “DMD,” “DMD gene,” and equivalents thereof refer to the DMD gene that encodes for the protein dystrophin. In addition, the terms “DMD” and “DMD gene” are used interchangeable, and both terms refer to the dystrophin gene.

### EXAMPLES

These examples are provided for illustrative purposes only and not to limit the scope of the claims provided herein.

### Example 1. Antisense Oligonucleotide Sequences and Synthesis

Phosphorodiamidate morpholino oligomers (PMO), phosphorothioate antisense oligonucleotides (PS ASO), and antisense oligonucleotides (ASOs) were synthesized.

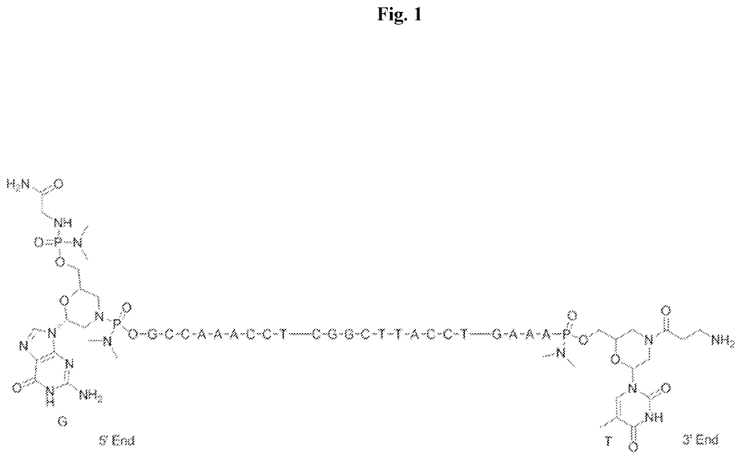

The PMO sequence was 5′GGCCAAACCTCGGCTTACCTGAAAT 3′ Primary amine (SEQ ID NO: 28) and can be seen in FIG. 1 with end nucleotides expanded. The PMO contains a C3-NH2 conjugation handle at the 3′ end of the molecule for conjugation. PMOs were fully assembled on solid phase using standard solid phase synthesis protocols and purified over HPLC.

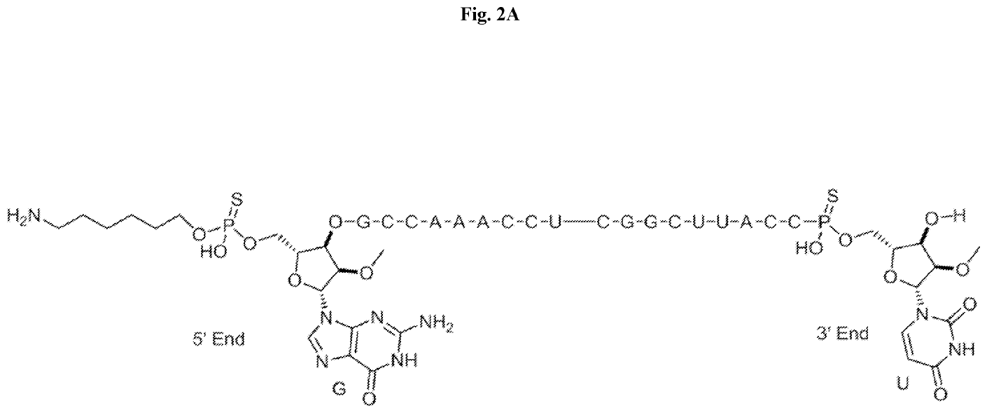

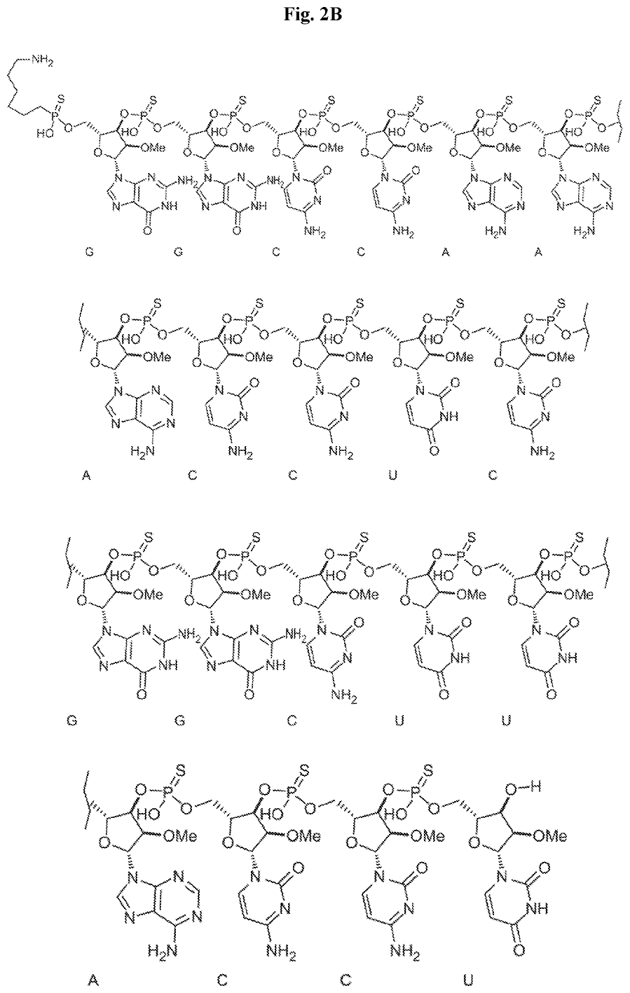

The PS ASO sequence was Amine-C6-GGCCAAACCUCGGCUUACCU (SEQ ID NO: 29) and can be seen in FIGS. 2A-2B with end nucleotides expanded. The structure of the PS ASO comprised a phosphate backbone that was 100% phosphorothioate linkages and all the ribose sugars contained a 2′ 2′OMe modification. The PS ASO also contained a C6-NH2 conjugation handle at the 5′ end of the molecule for conjugation. The PS ASOs were fully assembled on the solid phase using standard solid phase phosphoramidite chemistry and purified over HPLC.

ASOs were fully assembled on the solid phase using standard solid phase phosphoramidite chemistry and purified over HPLC. ASOs contained a C6-NH2 conjugation handle at the 5′ end of the molecule for conjugation.

### Example 2. Detection of DMD Exon Skipping

### Methods for Determining DMD Exon 23 Skipping in Differentiated C1C12 Cells

Mouse myoblast C2C12 cells were plated at 50,000-100,000/well in 24-well plates in 0.5 mL 10% FBS RPMI 1640 media and incubated at 37° C. with 5% CO2 overnight. On the second day, cells were switched to differentiation media (2% horse serum RPMI 1640 and 1 μM insulin) and incubated for 3-5 days. Following incubation, samples were added and incubated for 24 hours. After the sample treatment, 1 mL of fresh media (with no compounds) was changed every day for 2 more days. At 72 hours after the start of treatments, cells were harvested. RNAs were isolated using InviTrap RNA Cell HTS 96 Kit (B-Bridge International #7061300400) and reverse transcribed using High Capacity cDNA Reverse transcription Kit (ThermoFisher #4368813). PCR reactions were performed using DreamTaq™ PCR Mastermix (ThermoFisher #K1072). The primary PCR used primers in exon 20 (Ex20F 5′-CAGAATTCTGCCAATTGCTGAG) (SEQ ID NO: 30) and exon 26 (Ex26R 5′-TTCTTCAGCTTGTGTCATCC) (SEQ ID NO: 31) to amplify both skipped and unskipped molecules using the protocol in Table 3.

TABLE 3
PCR Protocol

| Hot Start | 95° C. for 2 minutes |
|---|---|
| Denaturation | 95° C. for 0.5 minute |
| Annealing of primers | 50° C. for 0.5 minute |
| Primer extension | 72° C. for 1 minute |
| Final extension | 72° C. for 5 minutes |
| Number of Cycles | 10 |

For the nested PCR, primary PCR reactions were diluted with water 100×, and 5 μl was used for nested PCR reaction (50 μl total reaction volume). Nested PCR used primers in exon 20 (Ex20F2: 5′-ACCCAGTCTACCACCCTATC) (SEQ ID NO: 32) and exon 25 (Ex25R: 5′-CTCTTTATCTTCTGCCCACCTT) (SEQ ID NO: 33) to amplify both skipped and unskipped molecules using the protocol in Table 4.

TABLE 4
Nested PCR Protocol

| Hot Start | 95° C. for 2 minutes |
|---|---|
| Denaturation | 95° C. for 0.5 minute |
| Annealing of primers | 50° C. for 0.5 minute |
| Primer extension | 72° C. for 1 minute |
| Final extension | 72° C. for 5 minutes |
| Number of Cycles | 35 |

PCR reactions were analyzed using 4% TAE agarose gels. The wild-type (WT) DMD product had an expected size of 788 base pairs and the skipped DMD A23 of 575 base pairs.

### Animals

All animal studies were conducted following protocols in accordance with the Institutional Animal Care and Use Committee (IACUC) at Explora BioLabs, which adhere to the regulations outlined in the USDA Animal Welfare Act as well as the “Guide for the Care and Use of Laboratory Animals” (National Research Council publication, 8th Ed., revised in 2011). All mice were obtained from either Charles River Laboratories or Harlan Laboratories.

### In Vivo Mouse Model

WT CD-1 mice (4-6 weeks old) were dosed via intravenous (iv) injection with the indicated antisense conjugates (ASCs) and doses. The “naked” PMO or ASO were dosed via intramuscular injection at the indicated doses. After 4, 7, or 14 days, heart and gastrocnemius muscle tissues were harvested and snap-frozen in liquid nitrogen. RNAs were isolated with Trizol and RNeasy Plus 96 Kit (Qiagen, #74192) and reversed transcribed using High Capacity cDNA Reverse transcription Kit (ThermoFisher #4368813). Nested PCR reactions were performed as described. PCR reactions were analyzed in 4% (or 1%) TAE agarose gels which were quantitated by densitometry.

To confirm exon 23 skipping in treated mice, DNA fragments were isolated from the 4% agarose gels and sequenced.

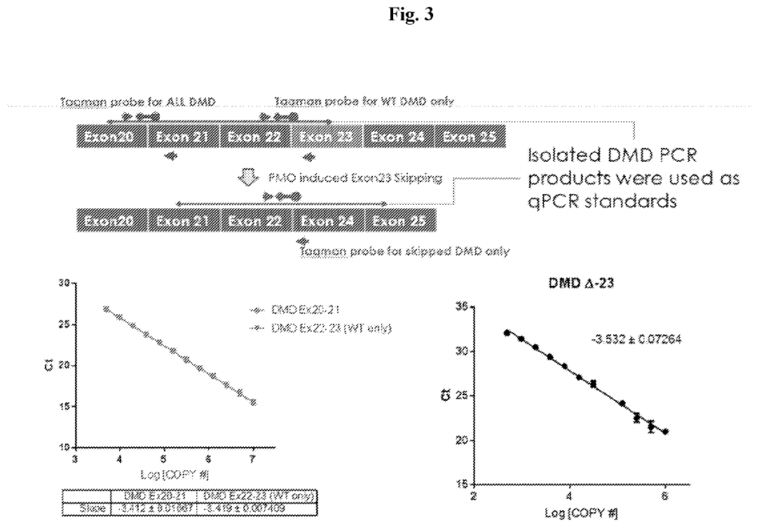

To quantitatively determine the skipped DMD mRNA copy number, qPCR primer/probe sets were designed to quantify skipped and WT DMD mRNA (FIG. 3). qPCR quantification standards were designed and produced via PCR using designed PCR primers as seen in Table 4. For the qPCR standard for WT and DMD, following PCR a 733 base pair fragment was isolated from the agarose gel. For qPCR standard for skipped DMA, the nested primers were used.

The amplification efficiency of the qPCR primer/probes (Table 5) were determined to be within 10% of expected efficiency. qPCR reactions were performed in QuantStudio 7 and Taqman™ PCR Universal Mastermix II (ThermoFisher #4440041) according to manufacturer's instructions.

TABLE 5
qPCR primers and probes for detecting
exon skipping

| | SEQ ID | Primer/ | |
|---|---|---|---|
| | NO | Probe | Sequence |
| DMD A-23, for | 34 | Forward | 5′ GCGCTAT |
| Ex23 skipping | | Primer | CAGGAGACAA |
| | | | TGAG |
| | 35 | Reverse | 5′ GTTTTTA |
| | | Primer | TGTGATTCTG |
| | | | TAATTTCCC |
| | 36 | Probe | 5′ CTCTCTG |
| | | | TACCTTATCT |
| | | | TAGTGTT |
| DMD Ex22-23, | 37 | Forward | 5′ TGGAGGA |
| for WT DMD | | Primer | GAGACTCGGG |
| only | | | AAA |
| | 38 | Reverse | 5′ TTGAAGC |
| | | Primer | CATTTTGTTG |
| | | | CTCTTT |
| | 39 | Probe | 5′ ACAGGCT |
| | | | CTGCAAAGT |
| DMD Ex20-21, | 40 | Forward | 5′ AACAGAT |
| for All DMD | | Primer | GACAACTACT |
| | | | GCCGAAA |
| | 41 | Reverse | 5′ TTGGCTC |
| | | Primer | TGATAGGGTG |
| | | | GTAGAC |
| | 42 | Probe | 5′ CTTGTTG |
| | | | AAAACCC |
| qPCR | 43 | Forward | 5′ TGAGGGT |
| standard | | Primer | GTTAATGCTG |
| for WT | | | AAAGTA |
| and all | 44 | Reverse | 5′ CACCAAC |
| DMD | | Primer | TGGGAGGAAA |
| | | | GTT |

### Example 3: Conjugate Synthesis

### Analytical and Purification Methods

Analytical and purification methods were performed according to Tables 6-12.

TABLE 6
Size exclusion chromatography (SEC) methods

| Size Exclusion | | | |
|---|---|---|---|
| Chromatography | | Mobile | |
| (SEC) Method | Column | Phase | Flow Rate |
| method 1 | TOSOH Biosciences, | 150 mM | 1.0 mL/minute |
| | TSKgelG3000SW XL, | phosphate | for 20 minutes |
| | 7.8 × 300 mm, 5 μM | buffer | |
| method 2 | TOSOH Biosciences, | PBS pH 7.4 | 1.0 mL/minute |
| | TSKgelG3000SW, | | for 180 minutes |
| | 21.5 × 600 mm, 5 μM | | |

TABLE 7
Hydrophobic interaction chromatography (HIC) method 1

| | | Gradient | | |
|---|---|---|---|---|
| | | Column | | |
| Column | Solvent | Volume | % A | % B |
| GE, HiScreen | Solvent A: 50 mM phosphate | 1.00 | 95 | 5 |
| Butyl HP, | buffer, 0.8M Ammonium | 30 | 0 | 100 |
| 4.7 mL | Sulfate, pH 7.0 | 5 | 0 | 100 |
| | Solvent B: 80% 50 mM | | | |
| | phosphate buffer, 20% IPA, | | | |
| | pH 7.0 | | | |
| | Flow Rate: 1.0 mL/minute | | | |

TABLE 8
Hydrophobic interaction chromatography (HIC) method 2

| | | Gradient | | |
|---|---|---|---|---|
| Column | Solvent | Time | % A | % B |
| Thermo Scientific, | Solvent A: 100 mM phosphate | 0.00 | 100 | 0 |
| MAbPac HIC-20, | buffer, 1.8M Ammonium | 2.00 | 100 | 0 |
| 4.6 mm ID × 10 cm, | Sulfate, pH 7.0 | 22.00 | 0 | 100 |
| 5 um | Solvent B: 80% 100 mM | 25.00 | 0 | 100 |
| | phosphate buffer, 20% IPA, | 26.00 | 100 | 0 |
| | pH 7.0 | 30.00 | 100 | 0 |
| | Flow Rate: 0.7 mL/minute | | | |

TABLE 9
Hydrophobic interaction chromatography (HIC) method 3

| | | Gradient | | |
|---|---|---|---|---|
| | | Column | | |
| Column | Solvent | Volume | % A | % B |
| GE, HiScreen | Solvent A: 50 mM phosphate | 1 | 100 | 0 |
| Butyl HP, | buffer, 0.8M Ammonium | 25 | 0 | 80 |
| 4.7 mL | Sulfate, pH 7.0 | 1 | 0 | 100 |
| | Solvent B: 80% 50 mM | 2 | 0 | 100 |
| | phosphate buffer, 20% IPA, | | | |
| | pH 7.0 | | | |
| | Flow Rate: 1.0 mL/minute | | | |

TABLE 10
Hydrophobic interaction chromatography (HIC) method 4

| | | Gradient | | |
|---|---|---|---|---|
| Column | Solvent | Time | % A | % B |
| Thermo Scientific, | Solvent A: 100 mM phosphate | 0.00 | 100 | 0 |
| MAbPac HIC-20, | buffer, 1.8M Ammonium | 5.00 | 100 | 0 |
| 4.6 mm ID × 10 cm, | Sulfate, pH 7.0 | 20.00 | 0 | 100 |
| 5 um | Solvent B: 80% 100 mM | 25.00 | 0 | 100 |
| | phosphate buffer, 20% IPA, | 26.00 | 100 | 0 |
| | pH 7.0 | 30.00 | 100 | 0 |
| | Flow Rate: 0.5 mL/minute | | | |

TABLE 11
Strong anion exchange chromatography (SAX) method 1

| | | Gradient | | |
|---|---|---|---|---|
| | | Column | | |
| Column | Solvent | Volume | % A | % B |
| Tosoh Bioscience, | Solvent A: 20 mM TRIS | 0.5 | 100 | 0 |
| TSK Gel SuperQ- | buffer, pH 8.0; | 0.5 | 80 | 20 |
| 5PW, 21.5 mm ID × | Solvent B: 20 mM TRIS, | 17 | 20 | 80 |
| 15 cm, 13 um | 1.5M NaCl, pH 8.0 | 0.5 | 0 | 100 |
| | Flow Rate: 6.0 mL/minute | 0.5 | 0 | 100 |

TABLE 12
Strong anion exchange chromatography (SAX) method 2

| | | Gradient | | |
|---|---|---|---|---|
| Column | Solvent | Time | % A | % B |
| Thermo Scientific, | Solvent A: 80% 10 mM TRIS | 0.0 | 90 | 10 |
| ProPac ™ SAX-10, | pH 8, 20% ethanol | 3.00 | 90 | 10 |
| Bio LC ™, 4 × 250 | Solvent B: 80% 10 mM TRIS | 17.00 | 0 | 100 |
| mm | pH 8, 20% ethanol, 1.5M | 21.00 | 0 | 100 |
| | NaCl | 22.00 | 90 | 10 |
| | Flow Rate: 0.75 mL/minute | 25.00 | 90 | 10 |

Anti-mouse transferrin receptor antibody or anti-CD71 mAb that was used was a rat IgG2a subclass monoclonal antibody that binds mouse CD71 or mouse transferrin receptor 1 (mTfR1). The antibody was produced by BioXcell and it is commercially available (Catalog #BE0175).

### Anti-CD71 Antibody Morpholino Antisense Oligonucleotide Conjugate (Anti-CD71 mAb-PMO)

Anti-CD71 mAb-PMO Conjugation

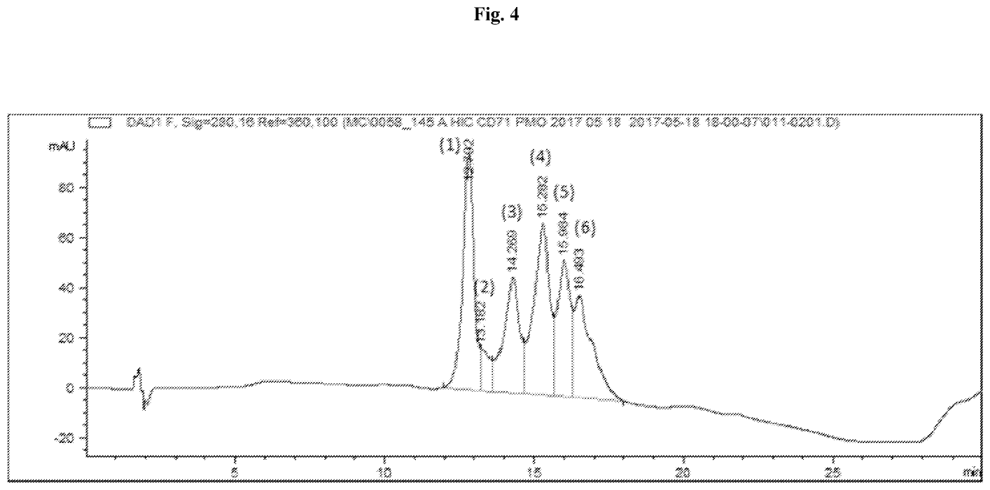

Anti-CD71 antibody (10 mg/mL) in borate buffer (25 mM sodium tetraborate, 25 mM NaCl, 1 mM Diethylene triamine pentaacetic acid, pH 8.0) was reduced by adding 4 equivalents of tris(2-carboxyethyl)phosphine (TCEP) in water and incubating at 37° C. for 4 hours. 4(N-Maleimidomethyl) cyclohexanecarboxylic acid N-hydroxysuccinimide ester (SMCC) was coupled to the primary amine on the 3′ end of the phosphorodiamidate morpholino oligomer (PMO) by incubating the PMO (50 mg/mL) in DMSO with 10 equivalents of SMCC (10 mg/mL) in DMSO for one hour. Unconjugated SMCC was removed by ultrafiltration using Amicon Ultra-15 centrifugal filter units with a MWCO of 3 kDa. The PMO-SMCC was washed three times with acetate buffer (10 mM sodium acetate, pH 6.0) and used immediately. The reduced antibody was mixed with 2.25 equivalents of PMO-SMCC and incubated overnight at 4° C. The pH of the reaction mixture was then reduced to 7.5, and 8 equivalents of N-Ethylmaleimide was added to the mixture at room temperature for 30 minutes to quench unreacted cysteines. Analysis of the reaction mixture by hydrophobic interaction chromatography (HIC) method 2 showed antibody-PMO conjugates along with unreacted antibody and PMO (FIG. 4). FIG. 4 shows a chromatogram of anti-CD71 mAb-PMO reaction mixture produced with HIC method 2 showing free antibody peak (1), free PMO (2), DAR 1 (3), DAR 2 (4), DAR 3 (5), DAR ≥3 (6). “DAR” refers to a drug-to-antibody ratio. The number in parentheses refers to the peak in the chromatogram.

### Purification

The reaction mixture was purified with an AKTA Explorer FPLC using HIC method 1. Fractions containing conjugates with a drug to antibody ratio of one (DAR 1) and two (DAR 2) were combined and concentrated with Amicon Ultra-15 centrifugal filter units with a MWCO of 50 kDa separately from conjugates with a DAR greater than 2. Concentrated conjugates were buffer exchanged with PBS (pH 7.4) using Amicon Ultra-15 centrifugal filter units prior to analysis.

### Analysis of the Purified Conjugate

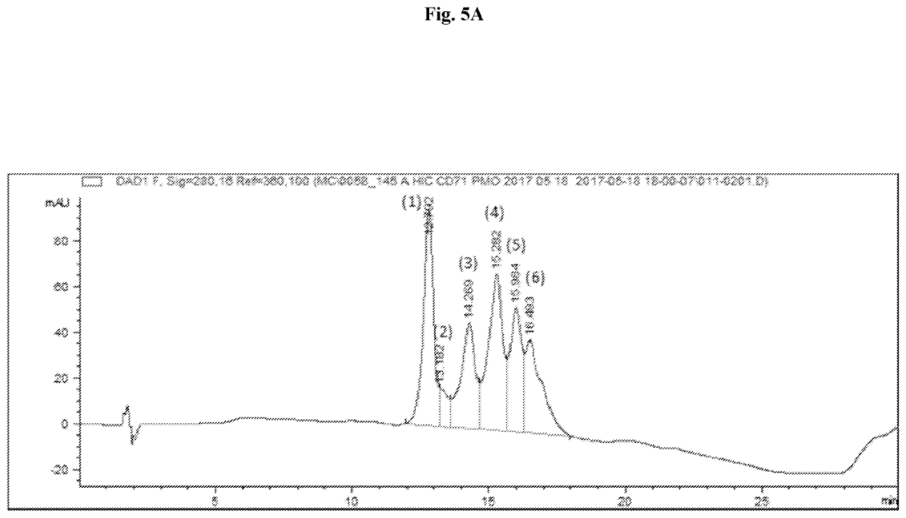

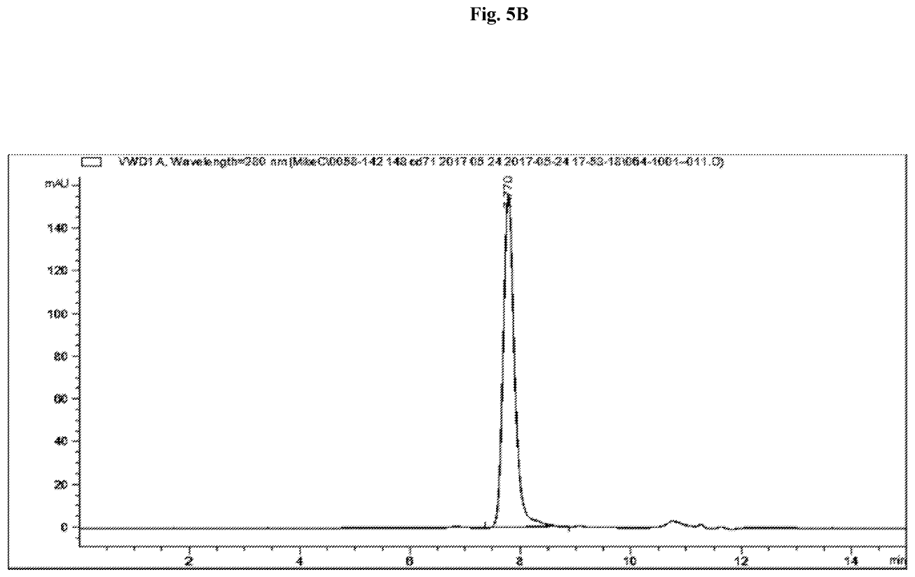

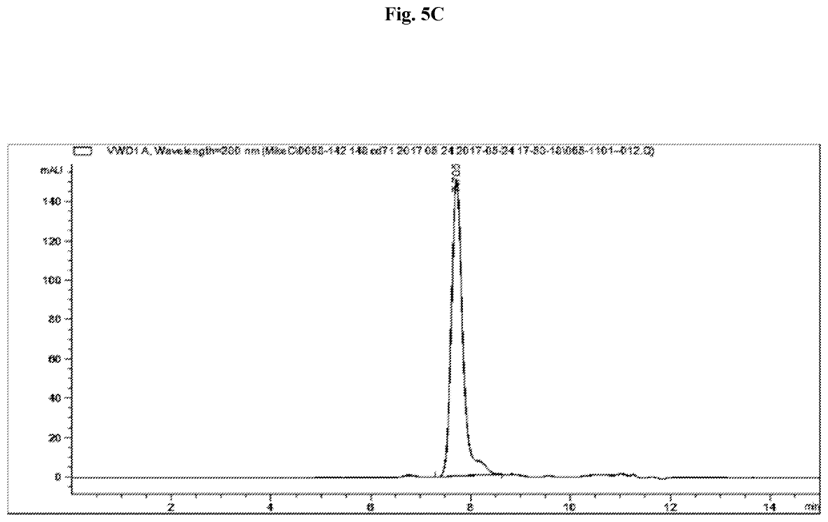

The isolated conjugates were characterized by size exclusion chromatography (SEC) and HIC. SEC method 1 was used to confirm the absence of high molecular weight aggregates and unconjugated PMOs (FIGS. 5A-5C). FIG. 5A shows a chromatogram of anti-CD71 mAb produced using SEC method 1. FIG. 5B shows a chromatogram of anti-CD71 mAb-PMO DAR 1,2 produced using SEC method 1. FIG. 5C shows a chromatogram of anti-CD71 mAb-PMO DAR greater than 2 produced using SEC method 1. “DAR” refers to a drug-to-antibody ratio.

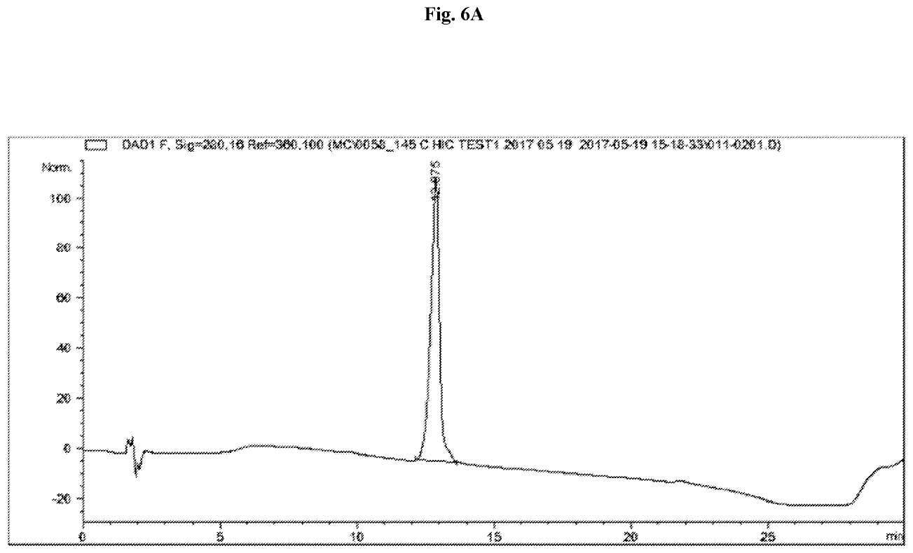

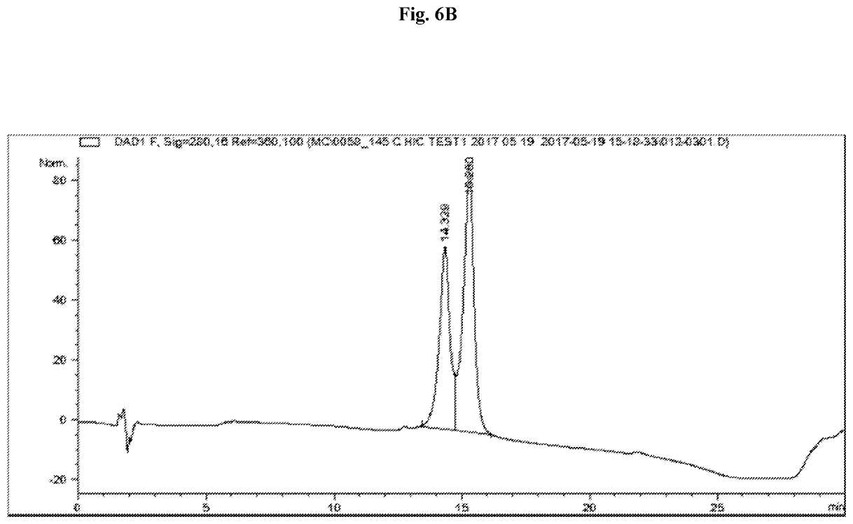

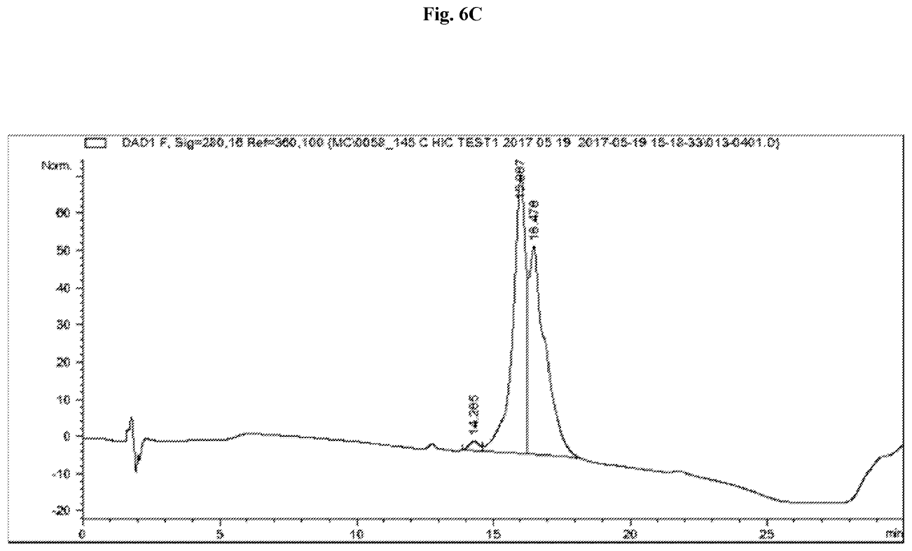

The purity of the conjugate was assessed by analytical HPLC using HIC method 2 (FIGS. 6A-6C). FIG. 6A shows a chromatogram of anti-CD71 mAb produced using HIC method 2. FIG. 6B shows a chromatogram of purified anti-CD71 mAb-PMO DAR 1,2 conjugate produced using HIC method 2. FIG. 6C shows a chromatogram of purified anti-CD71 mAb-PMO DAR >2 conjugate produced using HIC method 2. The 260/280 nm UV absorbance ratio of each sample was compared to a standard curve of known ratios of PMO and antibody to confirm DAR. The DAR 1,2 sample had an average DAR of ˜1.6 while the DAR greater than 2 sample had an average DAR of ˜3.7. “DAR” refers to a drug-to-antibody ratio.

### Anti-CD71 Fab Morpholino Antisense Oligonucleotide Conjugate (Anti-CD71 Fab-PMO)

Antibody Digestion with Pepsin

Anti-CD71 antibody (5 mg/mL) in 20 mM acetate buffer (pH 4.0) was incubated with immobilized pepsin for 3 hours at 37° C. The resin was removed and the reaction mixture was washed with PBS (pH 7.4) using Amicon Ultra-15 centrifugal filter units with a MWCO of 30 kDa. The retentate was collected and purified using size exclusion chromatography (SEC) method 2 to isolate the F(ab′)2 fragment.

### Anti-CD71 (Fab)-PMO Conjugation

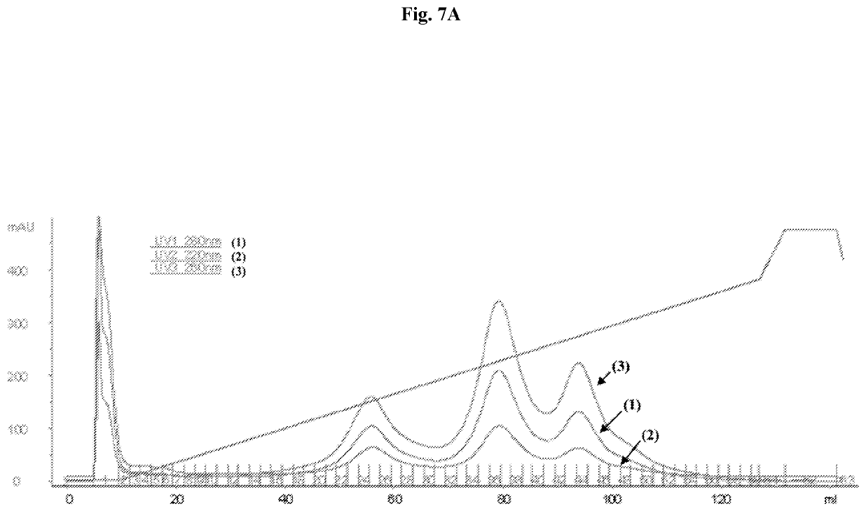

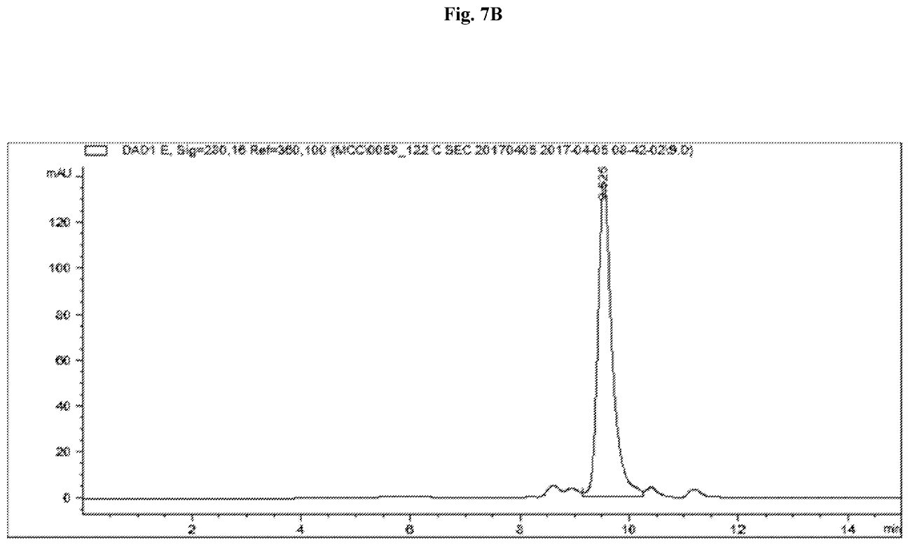

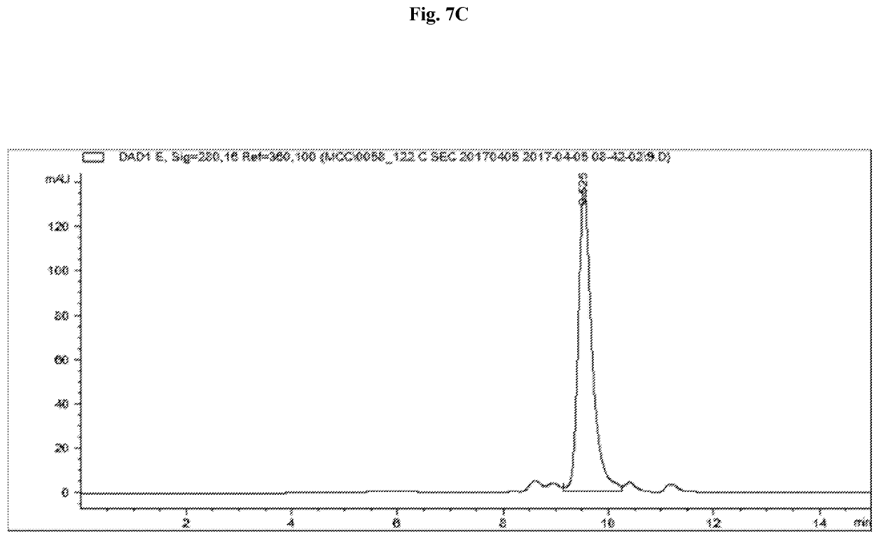

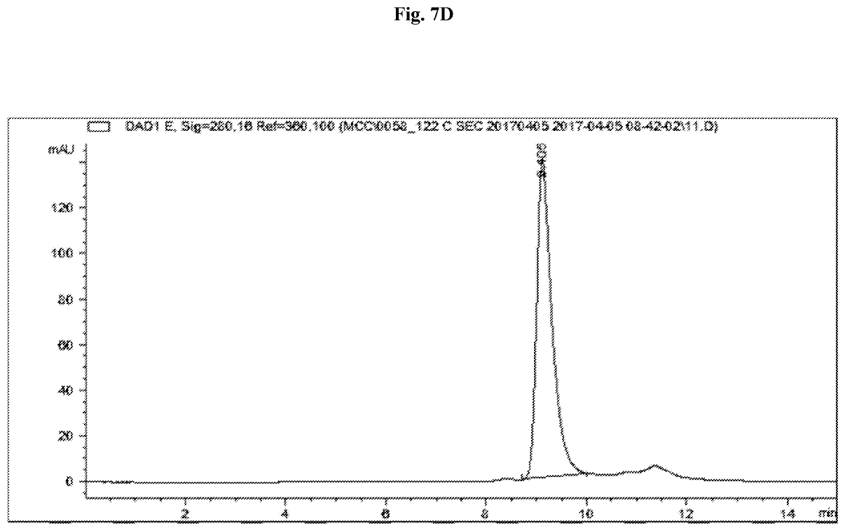

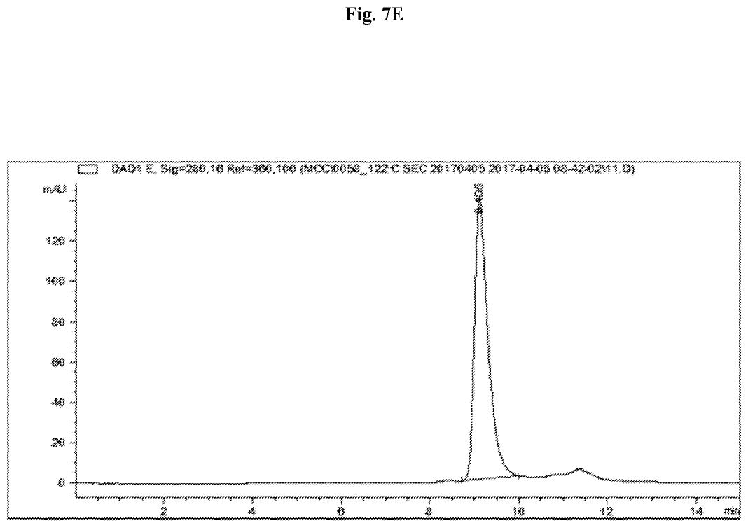

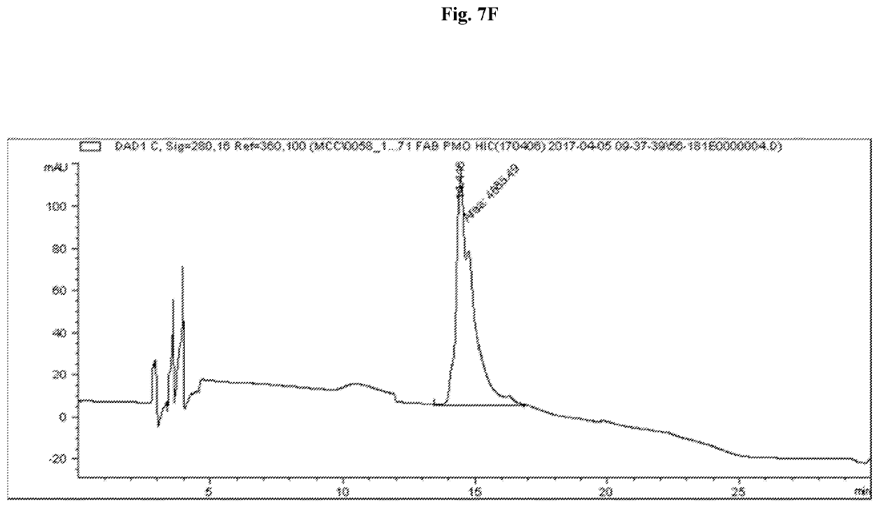

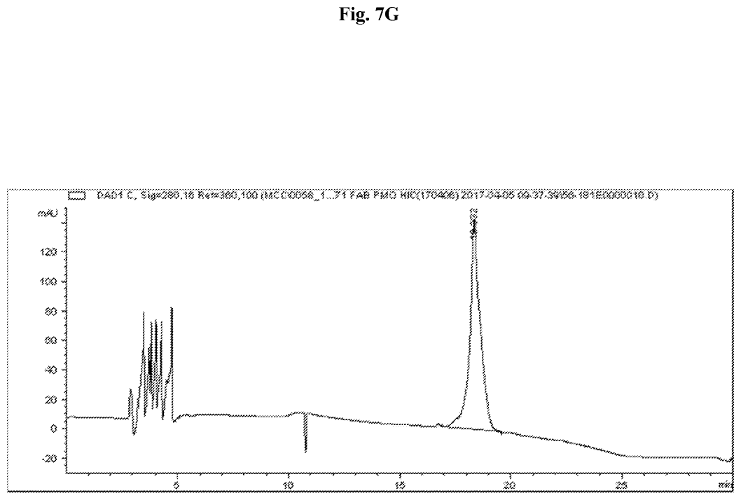

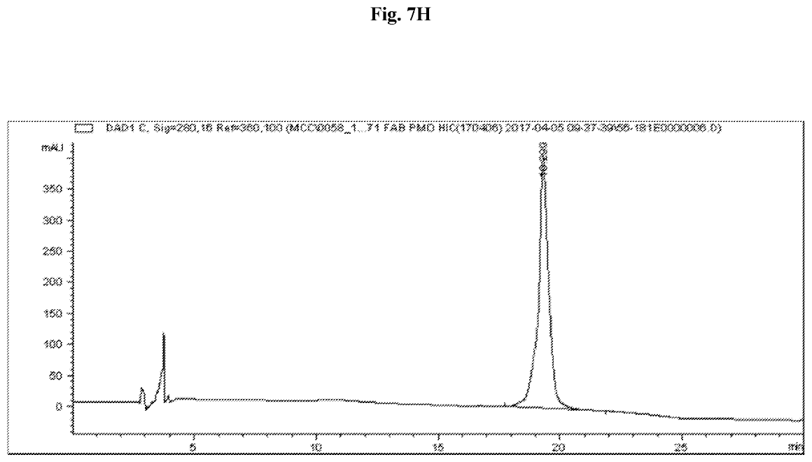

The F(ab′)2 fragment (15 mg/mL) in borate buffer (pH 8.0) was reduced by adding 10 equivalents of TCEP in water and incubating at 37° C. for 2 hours. SMCC was added to the primary amine on the 3′ end of the PMO by incubating the PMO (50 mg/mL) in DMSO with 10 equivalents of SMCC (10 mg/mL) in DMSO for 1 hour. Unconjugated SMCC was removed by ultrafiltration using Amicon Ultra-15 centrifugal filter units with a MWCO of 3 kDa. The PMO-SMCC was washed three times with acetate buffer (pH 6.0) and used immediately. The reduced F(ab′) fragment (Fab) was buffer exchanged into borate buffer (pH 8.0) using Amicon Ultra-15 Centrifugal Filter Units with a MWCO of 10 kDa, and 1.75 equivalents of PMO-SMCC was added and incubated overnight at 4° C. The pH of the reaction mixture was then reduced to 7.5, and 6 equivalents of N-Ethylmaleimide was added to the mixture at room temperature for 30 minutes to quench unreacted cysteines. Analysis of the reaction mixture by hydrophobic interaction chromatography (HIC) method 3 showed anti-CD71 (Fab)-PMO conjugates along with unreacted Fab (FIG. 7A). FIG. 7A shows a chromatogram of FPLC purification of anti-CD71 Fab-PMO using HIC method 3.

### Purification

The reaction mixture was purified with an AKTA Explorer FPLC using HIC method 3. Fractions containing conjugates with a DAR of one, two and three were combined and concentrated separately. Concentrated conjugates were buffer exchanged with PBS (pH 7.4) using Amicon Ultra-15 centrifugal filter units with a MWCO of 10 kDa prior to analysis.

### Analysis of the Purified Conjugate

The isolated conjugates were characterized by SEC, and HIC. SEC method 1 was used to confirm the absence of high molecular weight aggregates and unconjugated PMO. See FIGS. 7B-7E. FIG. 7B shows a chromatogram of anti-CD71 Fab produced using SEC method 1. FIG. 7C shows a chromatogram of anti-CD71 Fab-PMO DAR 1 conjugate produced using SEC method 1. FIG. 7D shows a chromatogram of anti-CD71 Fab-PMO DAR 2 conjugate produced using SEC method 1. FIG. 7E shows a chromatogram of anti-CD71 Fab-PMO DAR 3 conjugate produced using SEC method 1. The purity of the conjugate was assessed by analytical HPLC using HIC method 4. See FIGS. 7F-7I. FIG. 7F shows a chromatogram of anti-CD71 Fab produced using HIC method 4. FIG. 7G shows a chromatogram of anti-CD71 Fab-PMO DAR 1 conjugate produced using HIC method 4. FIG. 7H shows a chromatogram of anti-CD71 Fab-PMO DAR 2 conjugate produced using HIC method 4. FIG. 7I shows a chromatogram of anti-CD71 Fab-PMO DAR 3 conjugate produced using HIC method 4. “DAR” refers to drug-to-antibody ratio. The 260/280 nm UV absorbance ratio of each sample was compared to a standard curve of known ratios of PMO and Fab to confirm DAR.

Anti-CD71 Antibody Phosphorothioate Antisense Oligonucleotide Conjugate (Anti-CD71 mAb-PS ASO)
Anti-CD71 mAb-PSASO

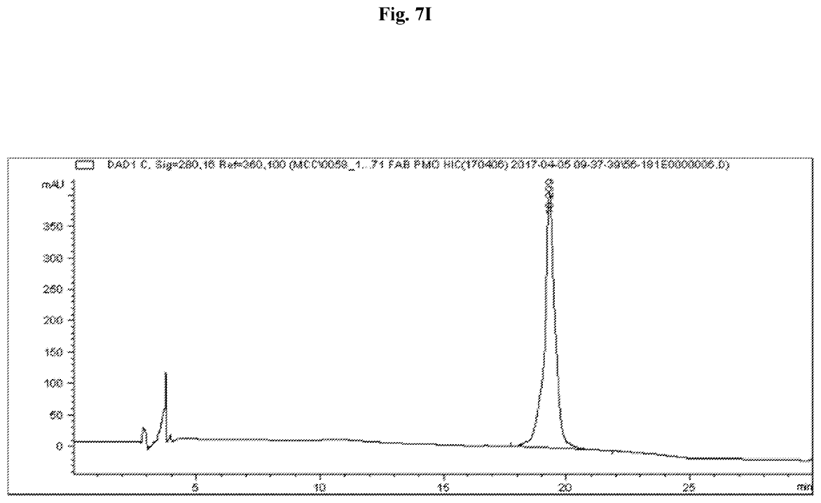

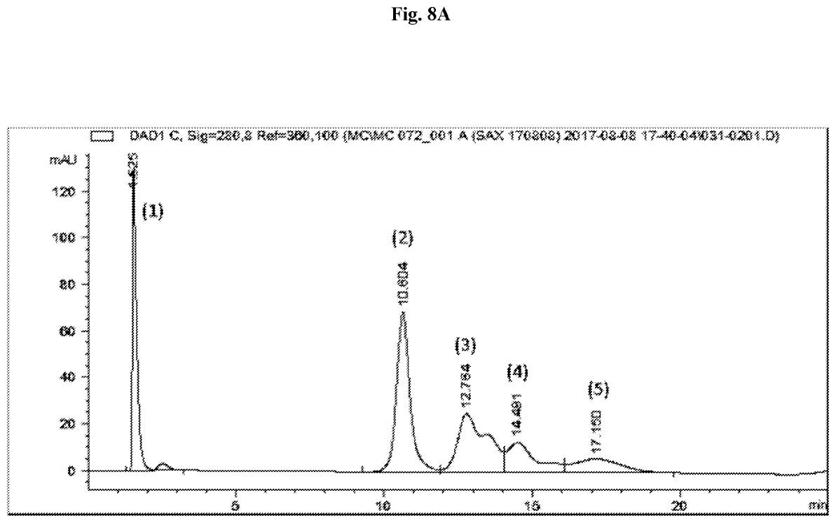

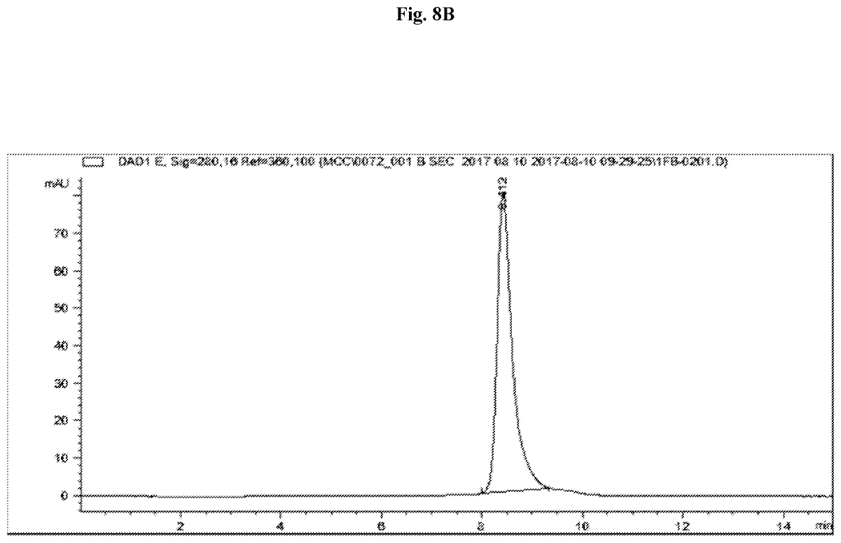

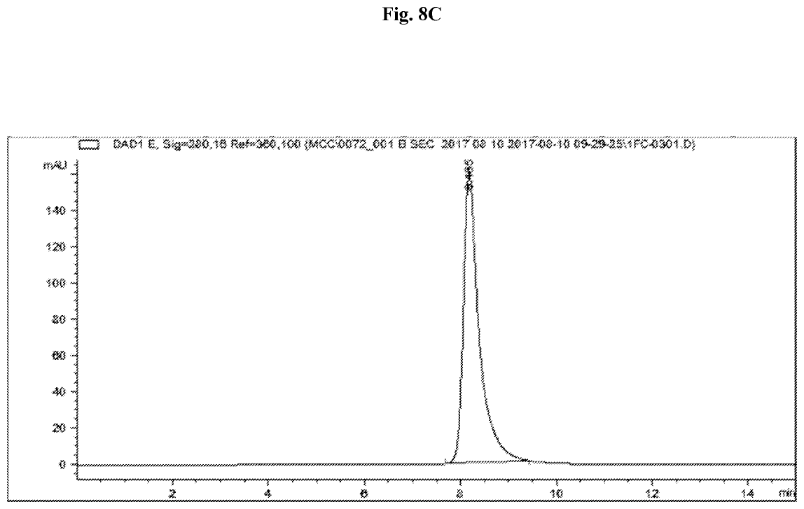

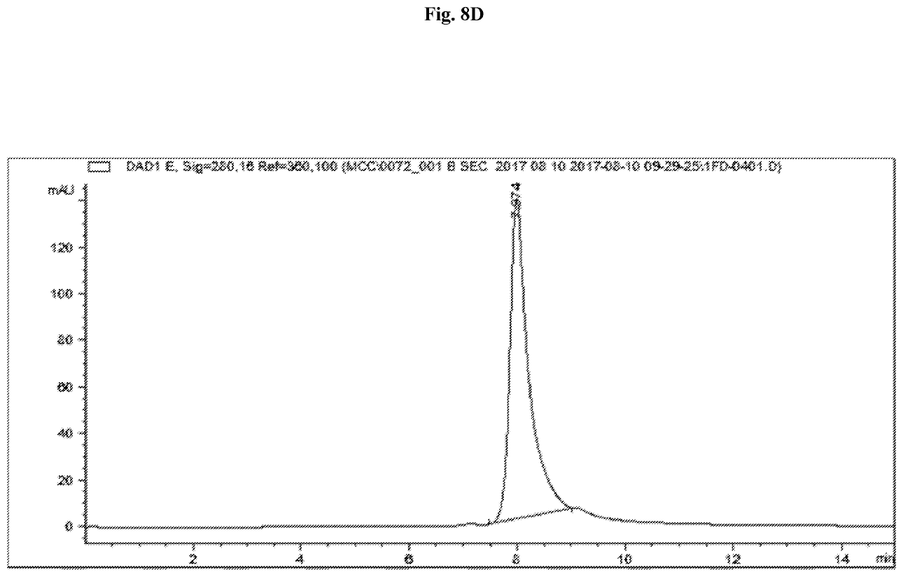

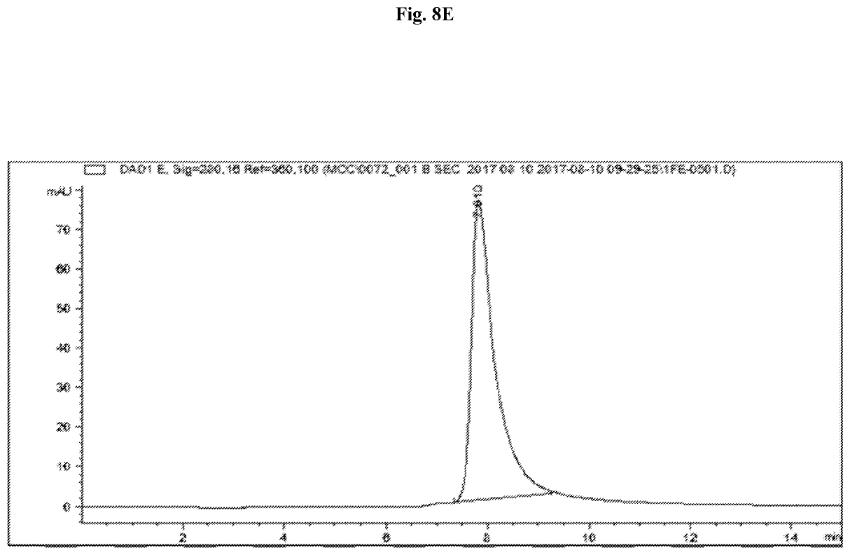

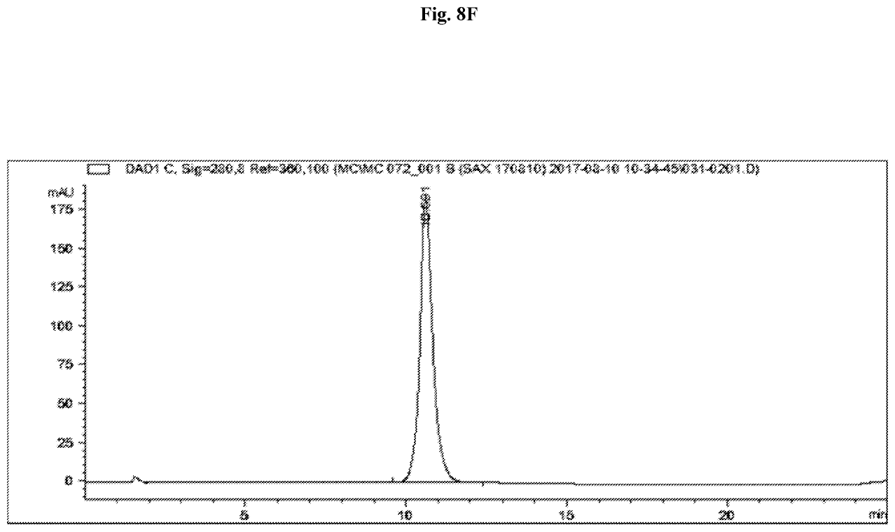

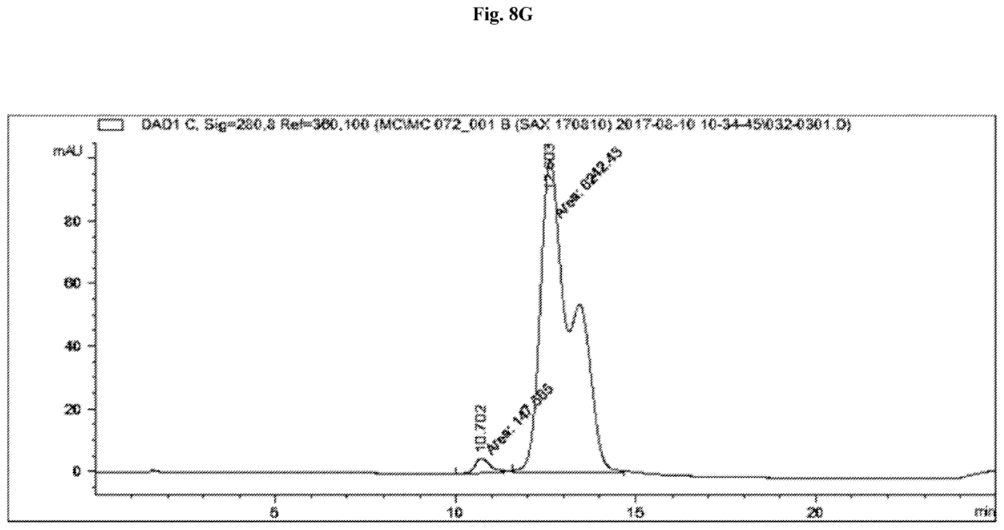

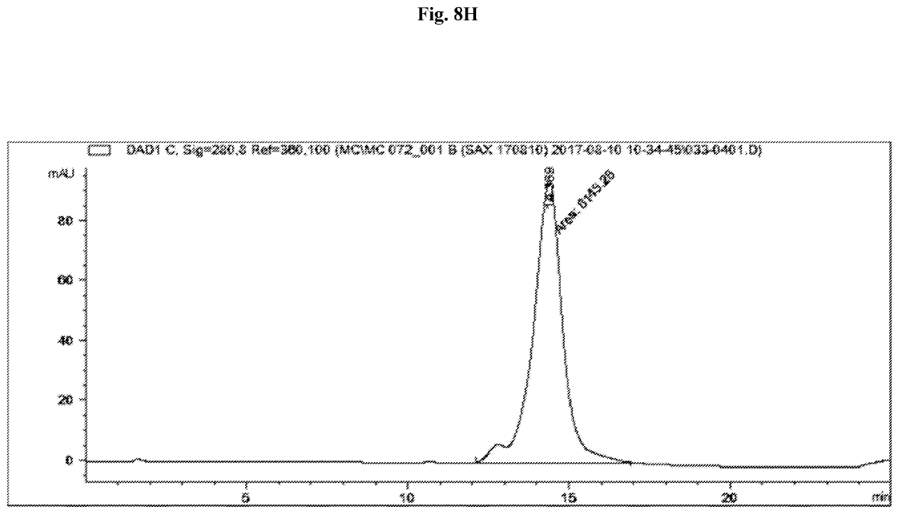

Anti-CD71 antibody (10 mg/mL) in borate buffer (pH 8.0) was reduced by adding 4 equivalents of TCEP in water and incubating at 37° C. for 4 hours. 4(N-Maleimidomethyl)cyclohexanecarboxylic acid N-hydroxysuccinimide ester (SMCC) was added to the primary amine on the 5′ end of the PS-ASO by incubating the PS ASO (50 mg/mL) in 1:1 mixture of 250 mM PB (pH 7.5) and DMSO with 10 equivalents of SMCC (10 mg/mL) in DMSO for 1 hour. Unconjugated SMCC was removed by ultrafiltration using Amicon Ultra-15 centrifugal filter units with a MWCO of 3 kDa. The PS ASO-SMCC was washed three times with acetate buffer (pH 6.0) and used immediately. The reduced antibody was mixed with 1.7 equivalents of PS ASO-SMCC and incubated overnight at 4° C. The pH of the reaction mixture was then reduced to 7.4, and 8 equivalents of N-Ethylmaleimide was added to the mixture at room temperature for 30 minutes to quench unreacted cysteines. Analysis of the reaction mixture by strong anion exchange chromatography (SAX) method 2 showed antibody-PS ASO conjugates along with unreacted antibody and ASO (FIG. 8A). FIG. 8A shows a chromatogram of anti-CD71 mAb-PS ASO reaction mixture produced with SAX method 2 showing free antibody peak (1), free PS ASO (5), DAR 1 (2), DAR 2 (3), DAR >2 (4). “DAR” refers to a drug-to-antibody ratio. The number in parentheses refers to the peak.

### Purification

The reaction mixture was purified with an AKTA Explorer FPLC using SAX method 1. Fractions containing conjugates with a drug-to-antibody ratio (DAR) of one, two and three were combined and concentrated separately and buffer exchanged with PBS (pH 7.4) using Amicon Ultra-15 centrifugal filter units with a MWCO of 50 kDa prior to analysis.

### Analysis of the Purified Conjugate

The isolated conjugates were characterized by size exclusion chromatography (SEC) and SAX. Size exclusion chromatography method 1 was used to confirm the absence of high molecular weight aggregates and unconjugated ASO. See FIGS. 8B-8E. FIG. 8B shows a chromatogram of anti-CD71 mAb produced using SEC method 1. FIG. 8C shows a chromatogram of anti-CD71 mAb-PS ASO DAR 1 conjugate produced using SEC method 1. FIG. 8D shows a chromatogram of anti-CD71 mAb-PS ASO DAR 2 conjugate produced using SEC method 1. FIG. 8E shows a chromatogram of anti-CD71 mAb-PS ASO DAR 3 conjugate produced using SEC method 1. The purity of the conjugate was assessed by analytical HPLC using SAX method 2. See FIGS. 8F-8H. FIG. 8F shows a chromatogram of anti-CD71 mAb-PS ASO DAR 1 conjugate produced using SAX method 2. FIG. 8G shows a chromatogram of anti-CD71 mAb-PS ASO DAR 2 conjugate produced using SAX method 2. FIG. 8H shows a chromatogram of anti-CD71 mAb-PS ASO DAR 3 conjugate produced using SAX method 2. The 260/280 nm UV absorbance ratio of each sample was compared to a standard curve of known ratios of ASO and antibody to confirm drug-to-antibody ratio (DAR).

### Example 4: In Vitro Activity of Anti-CD71 mAb-PMO Conjugate

The anti-CD71 mAb-PMO conjugate was made and characterized as described in Example 3. The conjugate was assessed for its ability to mediate exon skipping in vitro in differentiated C2C12 cells using nested PCR using methods similar to Example 2. Briefly, the potency of “naked” morpholino ASO (“PMO”) was compared to an anti-CD71 mAb-PMO conjugate at multiple concentrations with the relevant vehicle controls. Controls included vehicle (“Veh”), scramble morpholino at 50 uM (“Scr50”), and no antibody (“Neg-Ab”). The concentrations of PMO used included 50 uM, 1 uM, and 0.02 uM. The concentrations of anti-CD71 mAB-PMO DAR 1,2 used included 200 nM, 20 nM, and 2 nM. “DAR” refers to drug-to-antibody ratio.

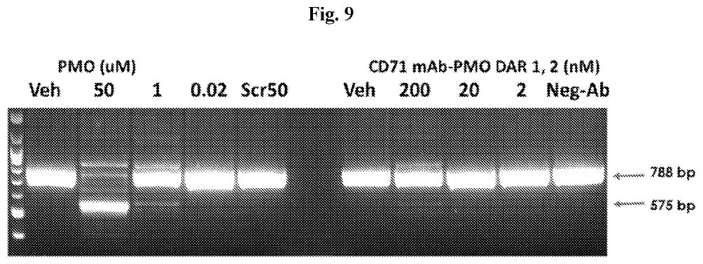

Following cDNA synthesis, two rounds of PCR amplification (primary and nested PCR) were used to detect exon-skipping. PCR reactions were analyzed in a 4% TAE agarose gel (FIG. 9).

Referring to FIG. 9, anti-CD71 mAb-PMO conjugate produced measurable exon 23 skipping in differentiated C2C12 cells and lower concentrations than the “naked” PMO control. The wild-type product had an expected size of 788 base pairs and the skipped DMD A23 of 575 base pairs.

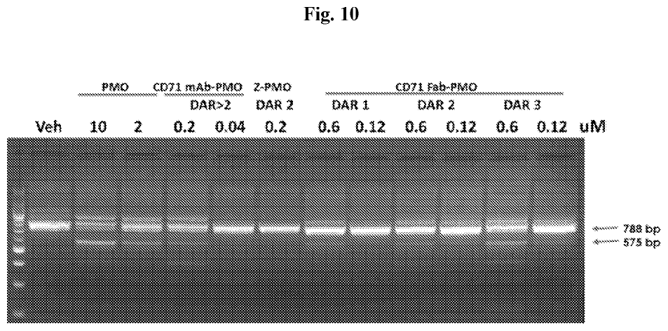

A second experiment included an anti-CD71 Fab-PMO conjugate and a PMO targeted with an anti-EGFR (“Z-PMO”) as a negative control (FIG. 10). The concentrations of PMO used included 10 uM and 2 uM. The concentrations of anti-CD71 mAb-PMO used included 0.2 uM and 0.04 uM. Anti-CD71 mAb-PMO had a DAR of 2. Z-PMO was used at a concentration of 0.2 uM and had a DAR of 2. Concentrations of anti-CD71 Fab-PMO included 0.6 uM and 0.12 uM. DAR of 1, 2, and 3 for anti-CD71 mAb-PMO at 0.6 uM and 0.12 uM were assayed.

Referring to FIG. 10, Receptor mediated uptake utilizing the transferrin receptor, the anti-CD71 mAb-PMO, and anti-CD71 Fab-PMO conjugates resulted in measurable exon 23 skipping in C2C12 cells and lower concentrations than the “naked” PMO control. There was no measurable exon 23 skipping from the Z-PMO at the concentration tested, which produced skipping from the anti-CD71 conjugates.

### Example 5. In Vitro Activity of Anti-CD71-ASO mAb PS Conjugate

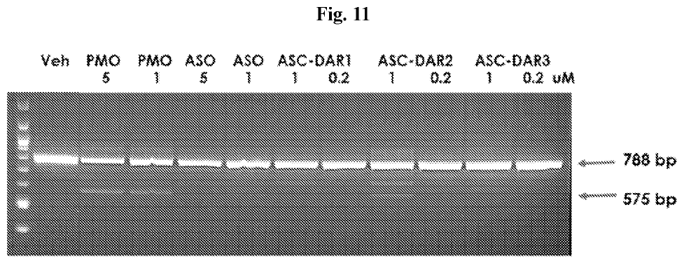

The anti-CD71 mAb-PS ASO conjugate was made and characterized as described in Example 3. The conjugate was assessed for its ability to mediate exon skipping in vitro in differentiated C2C12 cells using nested PCR using similar methods as described in Example 2. Briefly, the potency of “naked” phosphorothioate ASO (PS ASO) was compared to an anti-CD71 mAb-PS ASO conjugate at multiple concentrations, with the relevant vehicle control. Two rounds of PCR amplification (primary and nested PCR) were performed following cDNA synthesis to detect exon-skipping. PCR reactions were analyzed in a 4% TAE agarose gel (FIG. 11). FIG. 11 shows an agarose gel of PMO, ASO, conjugated anti-CD71 mAb-ASO of DAR1 (“ASC-DAR1”), conjugated anti-CD71 mAb-ASO of DAR2 (“ASC-DAR2”), and conjugated anti-CD71 mAb-ASO of DAR3 (“ASC-DAR3”). “PMO” and “ASO” refers to free PMO and ASO, unconjugated to antibody. “Veh” refers to vehicle only. The concentrations tested included 0.2, 1, and 5 micromolar (μM).

Referring to FIG. 11, the anti-CD71 mAb-PS ASO conjugate produced measurable exon 23 skipping in differentiated C2C12 cells and lower concentrations than the “naked” PS ASO control. The wild-type product had an expected size of 788 base pairs and the skipped DMD A23 of 575 base pairs.

### Example 6: In Vivo Activity of Anti-CD71 mAb-PMO Conjugate

The anti-CD71 mAb-PMO conjugate was made and characterized as described in Example 3. The conjugate anti-CD71 mAb-PMO DAR1,2 anti-CD71 and mAb-PMO DAR>2 were assessed for its ability to mediate exon skipping in vivo in wild-type CD-1 mice using similar methods as described in Example 2. “DAR” refers to drug-to-antibody ratio.

Mice were dosed via intravenous (iv) injection with the mAb, vehicle control, and antisense conjugates (ASCs) at the doses as provided in Table 12. “DAR” refers to drug-to-antibody ratio. The “naked” PMO was dosed via intramuscular injection into the gastrocnemius muscle at the doses provided in Table 13. After 4, 7, or 14 days, heart and gastrocnemius muscle tissues were harvested and snap-frozen in liquid nitrogen. RNAs were isolated, reversed transcribed and a nested PCR reactions were performed. PCR reactions were analyzed in 4% TAE agarose gels which were then quantitated by densitometry.

TABLE 13
In vivo study design

| | | | | | PMO:mAb | Harvest |
|---|---|---|---|---|---|---|
| | | | mAb dose | PMO Dose | Ratio | Time |
| Group | Test Article | N | (mg/kg) | (mg/kg) | (mol/mol) | (h) |
| 1 | anti-CD71 mAb-PMO, DAR1, 2 | 3 | 50 | 4.8 | 1.6 | 96 |
| 2 | anti-CD71 mAb-PMO, DAR1, 2 | 3 | 50 | 4.8 | 1.6 | 168 |
| 3 | anti-CD71 mAb-PMO, DAR1, 2 | 3 | 50 | 4.8 | 1.6 | 336 |
| 4 | anti-CD71 mAb-PMO, DAR > 2 | 3 | 50 | 10.5 | 3.7 | 96 |
| 5 | anti-CD71 mAb-PMO, DAR > 2 | 3 | 50 | 10.5 | 3.7 | 168 |
| 6 | anti-CD71 mAb-PMO, DAR > 2 | 3 | 50 | 10.5 | 3.7 | 336 |
| 7 | anti-CD71 mAb | 3 | 50 | | | 96 |
| 8 | anti-CD71 mAb | 3 | 50 | | | 168 |
| 9 | anti-CD71 mAb | 3 | 50 | | | 336 |
| 10 | PMO | 3 | 40 ug/inj. | | | 96 |
| 11 | PMO | 3 | 40 ug/inj. | | | 168 |
| 12 | PMO | 3 | 40 ug/inj. | | | 336 |
| 13 | Vehicle | 3 | | | | 96 |
| 14 | Vehicle | 3 | | | | 168 |
| 15 | Vehicle | 3 | | | | 336 |

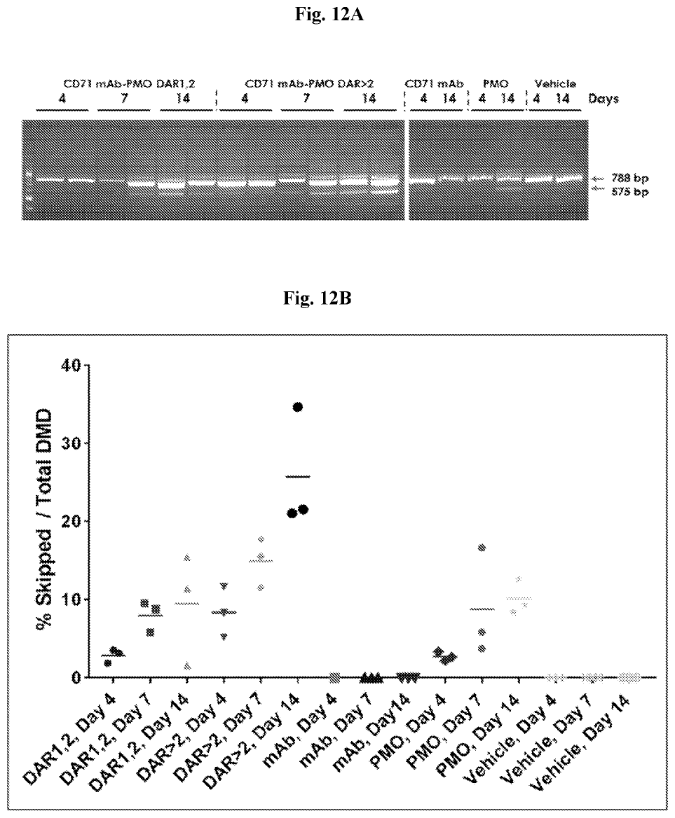

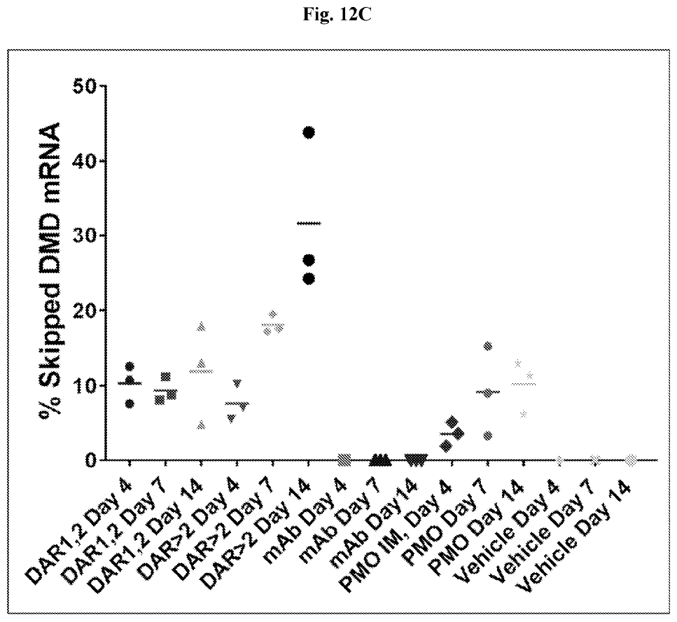

FIG. 12A shows a gel electrophoresis of gastrocnemius muscle samples from mice administered anti-CD71 mAb-PMO DAR 1,2, anti-CD71 mAb-PMO DAR>2, anti-CD71 mAb, PMO, and vehicle for 4, 7, or 14 days. The wild-type product had an expected size of 788 base pairs and the skipped DMD A23 of 575 base pairs. Anti-CD71 mAb-PMO DAR 1,2 and anti-CD71 mAb-PMO DAR>2 produced measurable exon 23 skipping in gastrocnemius muscle and lower concentrations than the “naked” PMO control. The intensity of the bands on the gel (FIG. 12A) was quantitated by densitometry as seen in FIG. 12B. FIG. 12C shows the quantification of in vivo exon skipping in wild-type mice gastrocnemius muscle using Tagman qPCR.

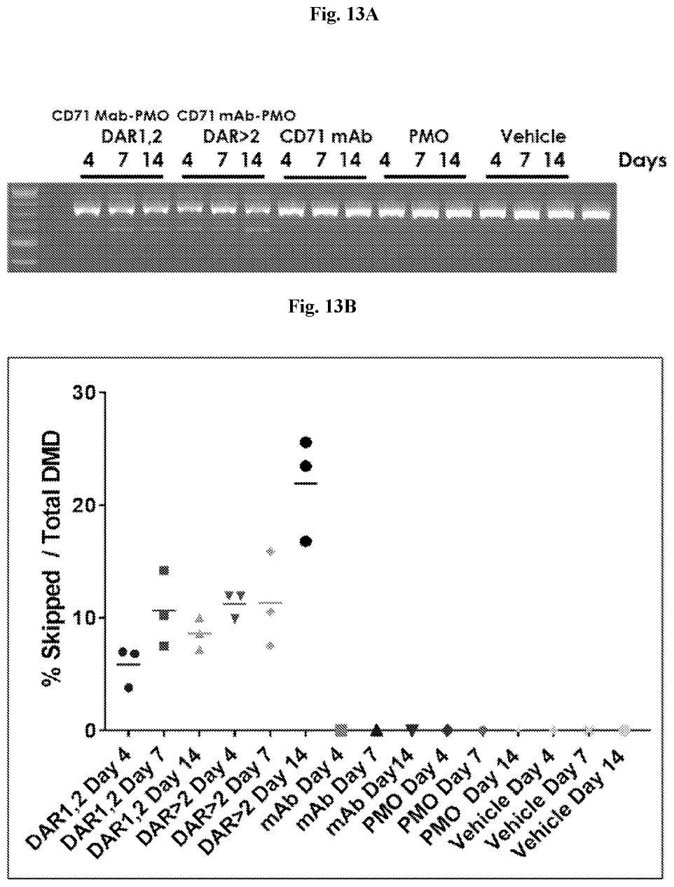

FIG. 13A shows a gel electrophoresis of heart samples from mice administered anti-CD71 mAb-PMO DAR 1,2, anti-CD71 mAb-PMO DAR>2, anti-CD71 mAb, PMO, and vehicle for 4, 7, or 14 days. The wild-type product had an expected size of 788 base pairs and the skipped DMD A23 of 575 base pairs. The intensity of the bands on the gel (FIG. 13A) was quantitated by densitometry as seen in FIG. 13B. Similar results as with the gastrocnemius muscle samples were obtained. Anti-CD71 mAb-PMO DAR 1,2 and anti-CD71 mAb-PMO DAR>2 produced measurable exon 23 skipping in gastrocnemius muscle and lower concentrations than the “naked” PMO control.

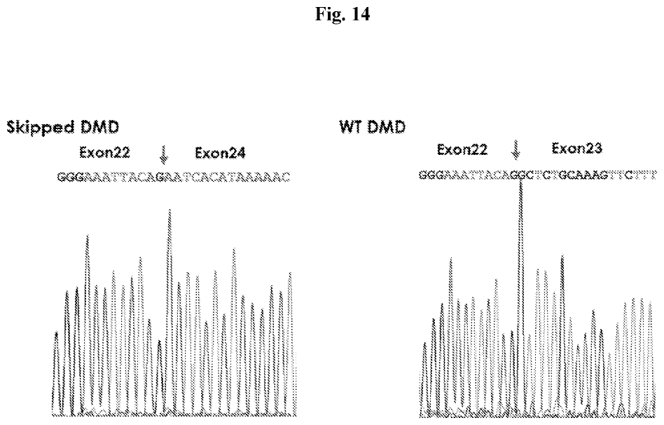

DNA fragments were then isolated from the 4% agarose gels and sequenced. The sequencing data confirmed the correct sequence in the skipped and wild-type products as seen in FIG. 14.

### Example 7. Antisense Oligonucleotide Sequences and Synthesis

The sequences in Table 14 were made targeting different exons in different genes.

TABLE 14
Sequences

| SEQ | | |
|---|---|---|
| ID | | PMO sequence |
| NO. | Target | (5′ to 3′) |
| 45 | Exon 23 in mouse | GGCCAAACCTCG |
| | dystrophin | GCTTACCTGAAA |
| | | T |
| 46 | Exon 2 in mouse | AGCCCATCTTCT |
| | myostatin | CCTGGTCCTGGG |
| | (MSTN) | AAGG |
| 47 | Exon 11 in mouse | ATCCTCTTTGGT |
| | phenylalanine | AACCTCACCTCA |
| | hydroxylase | C |
| | (PAH) | |
| 48 | KRAS-011 | TCGTCCACAAAA |
| | (human cancer) | TGATTCTGAATT |
| | | A |
| 49 | Scramble | CGGTGTGTGTAT |
| | | CATTCTCTAGTG |
| | | T |

### Example 8. In Vivo Activity of CD71 mAb-PMO Conjugate in Multiple Tissues

The CD71 mAb-PMO conjugates were made and characterized as described in Example 3. The conjugate (DAR3+) was assessed for its ability to mediate exon skipping in vivo in wild type CD-1 mice, see example 2 for full experimental details. In brief, mice were dosed via intravenous (iv) injection with vehicle control and indicated ASCs at the doses indicated, see FIG. 7A. After 7, 14 or 28 days, diaphragm, heart and gastrocnemius muscle tissues were harvested and snap-frozen in liquid nitrogen. RNAs were isolated, reversed transcribed, real-time qPCR and nested PCR reactions were performed as described in Example 2 using the appropriate primer/probe sets. PCR reactions were analyzed in 1% TAE agarose gels.

In vivo study design to assess the ability of the CD71 mAb-PMO conjugate to mediate exon 23 skipping in wild type mice is seen in Table 15.

TABLE 15
In vivo study design

| | | | mAb-PMO | PMO | PMO:mAb | Harvest |
|---|---|---|---|---|---|---|
| | | | mAb dose | Dose | Ratio | Time |
| Group | Test Article | N | (mg/kg) | (mg/kg) | (mol/mol) | (WEEKS) |
| 1 | Vehicle | 3 | | | | 1 |
| 2 | Vehicle | 3 | | | | 4 |
| 3 | CD71-scr, DAR3+ | 3 | 50 | 10 | 3.0 | 2 |
| 4 | CD71-DMD PMO, | 3 | 50 | 10 | 3.0 | 1 |
| | DAR3+ | | | | | |
| 5 | CD71-DMD PMO, | 3 | 50 | 10 | 3.0 | 2 |
| | DAR3+ | | | | | |
| 6 | CD71-DMD PMO, | 3 | 50 | 10 | 3.0 | 4 |
| | DAR3+ | | | | | |

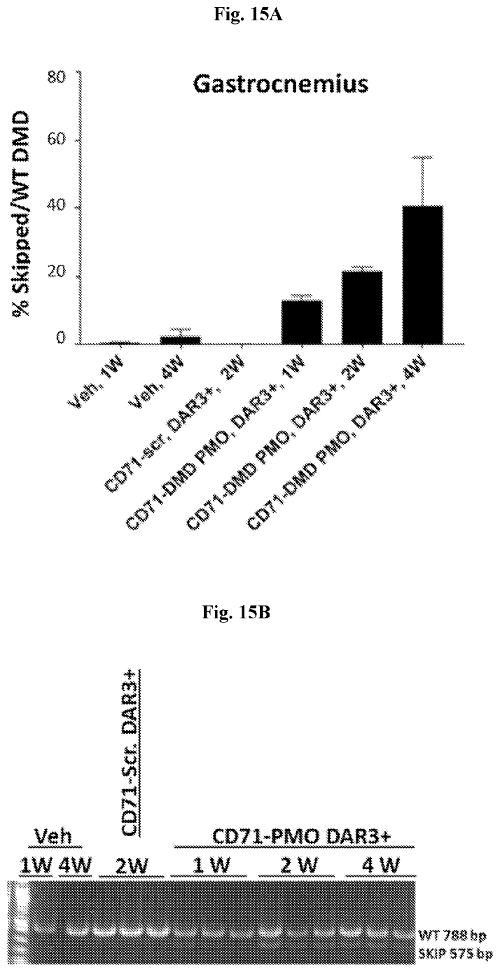

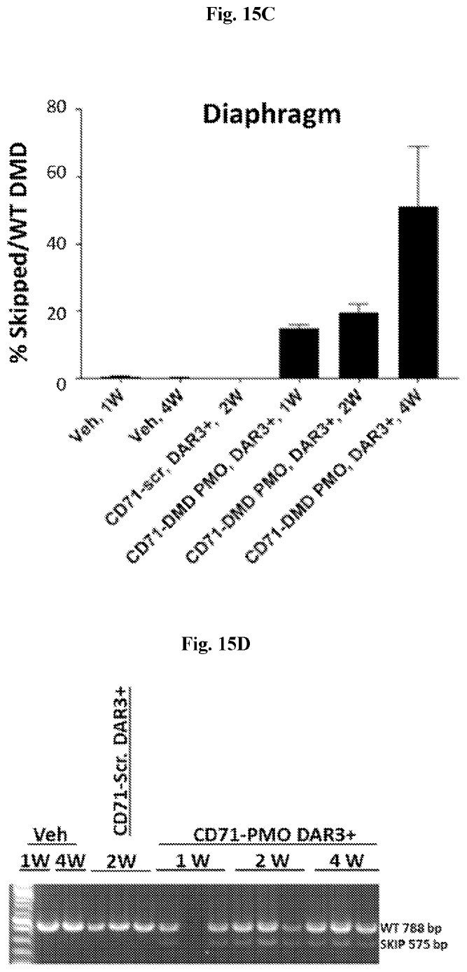

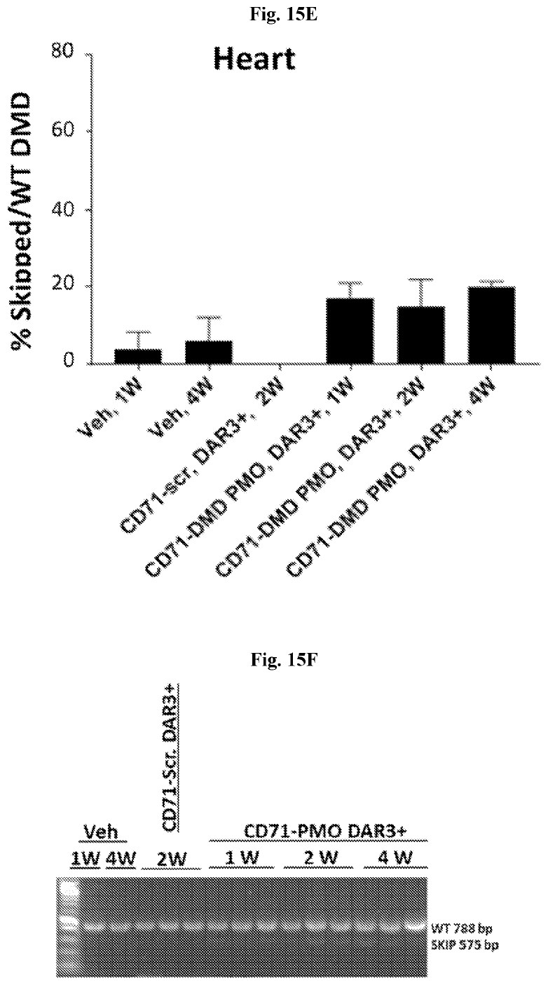

Referring to FIG. 15A, FIG. 15C, and FIG. 15E, in vivo exon skipping was measured in wild type mice in the gastrocnemius (FIG. 15A), diaphragm (FIG. 15C) and heart muscle (FIG. 15E) using Tagman qPCR. Referring to FIG. 15B, FIG. 15D, and FIG. 15F, the CD71 mAb-PMO conjugates produced measurable exon 23 skipping in gastrocnemius (FIG. 15B), diaphragm (FIG. 15D), and heart muscle (FIG. 15F) using nested PCR. The wild type product had an expected size of 788 bp, and the skipped DMD A23 had a size of 575 bp. The intensity of the bands on the gel was quantitated by densitometry, and the data are presented as the % of skipped product compared to wild-type dystrophin.

### Example 9. In Vivo Activity of CD71 mAb-PMO Conjugates Against Mouse MSTN

The CD71 mAb-PMO conjugate targeting exon 2 of mouse myostatin (5′ AGCCCATCTTCTCCTGGTCCTGGGAAGG) (SEQ ID NO: 46) was made and characterized as described in Example 3. The conjugates (DAR1/2 and DAR3+) were assessed for its ability to mediate exon skipping in vivo in wild type CD-1 mice using similar methods as described in Example 2. In brief, mice were dosed via intravenous (iv) injection with the mAb, vehicle control and indicated ASCs at the doses indicated as seen in Table 16.

TABLE 16
In vivo study design

| | | | mAb-PMO | | PMO:mAb | Harvest |
|---|---|---|---|---|---|---|
| | | | mAb dose | PMO Dose | Ratio | Time |
| Group | Test Article | N | (mg/kg) | (mg/kg) | (mol/mol) | (WEEKS) |
| 1 | CD71 mAb-PMO, | 3 | 50 | 5 | 1.5 | 1 |
| | DAR1/2 | | | | | |
| 2 | CD71 mAb-PMO, | 3 | 50 | 5 | 1.5 | 2 |
| | DAR1/2 | | | | | |
| 3 | CD71 mAb-PMO, | 3 | 50 | 5 | 1.5 | 4 |
| | DAR1/2 | | | | | |
| 4 | CD71 mAb-PMO, | 3 | 50 | 10 | 3.0 | 1 |
| | DAR3+ | | | | | |
| 5 | CD71 mAb-PMO, | 3 | 50 | 10 | 3.0 | 2 |
| | DAR3+ | | | | | |
| 6 | CD71 mAb-PMO, | 3 | 50 | 10 | 3.0 | 4 |
| | DAR3+ | | | | | |
| 7 | CD71-scr, DAR1/2 | 3 | 50 | 5 | 1.5 | 2 |
| 8 | CD71-scr, DAR3+ | 3 | 50 | 10 | 3.0 | 2 |
| 9 | Vehicle | 3 | | | | 1 |
| 10 | Vehicle | 3 | | | | 2 |
| 11 | Vehicle | 3 | | | | 4 |

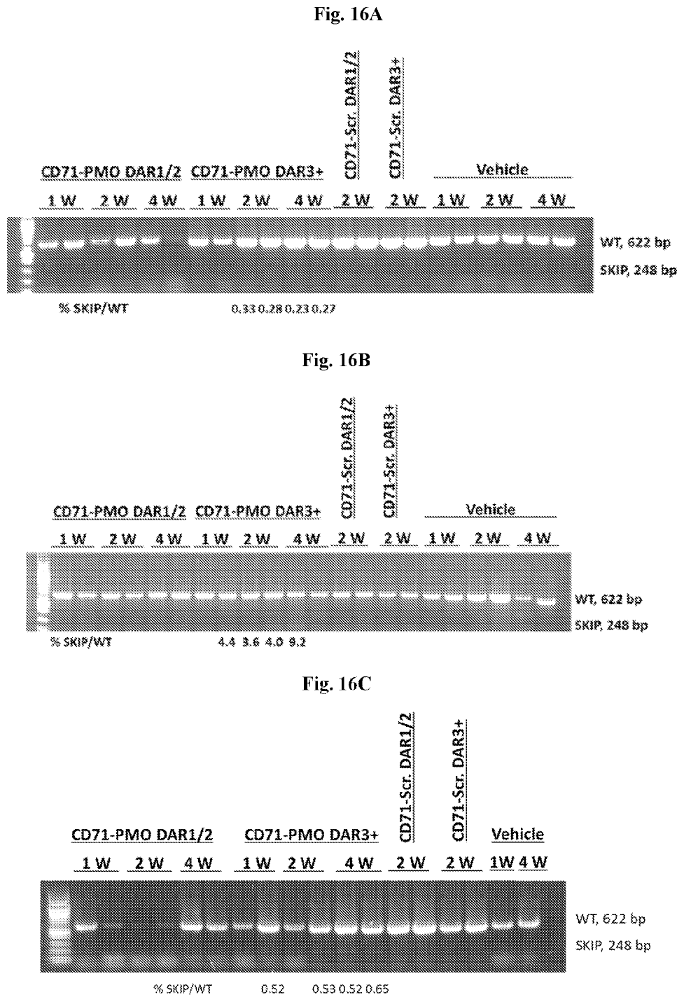

After 7, 14 or 28 days, diaphragm, heart and gastrocnemius muscle tissues were harvested and snap-frozen in liquid nitrogen. RNAs were isolated and reversed transcribed. PCR reactions were performed with forward primer (mMSTN-F1: 5′ CCTGGAAACAGCTCCTAACATC) (SEQ ID NO: 50) and reverse primer (mMSTN-R1: 5′CAGTCAAGCCCAAAGTCTCTC) (SEQ ID NO: 51) (hot start: 95° C. for 2 minutes, Denaturation at 95° C. for 45 seconds, Annealing of primers at 56° C. for 30 seconds, primer extension at 72° C. for 40 seconds for 35 cycles). PCR reactions were analyzed in a 1% TAE agarose gel as seen in FIGS. 16A-16C. The CD71 mAb-PMO conjugates produced measurable exon2 skipping in mouse diaphragm (FIG. 16A), heart (FIG. 16B) and gastrocnemius (FIG. 16C) muscle tissues. The wild type product had an expected size of 622 bp and the skipped MSTN A2 of 248 bp.

### Example 10. In Vitro Activity of ASGPR mAb-PMO Conjugates Against the PAH Gene

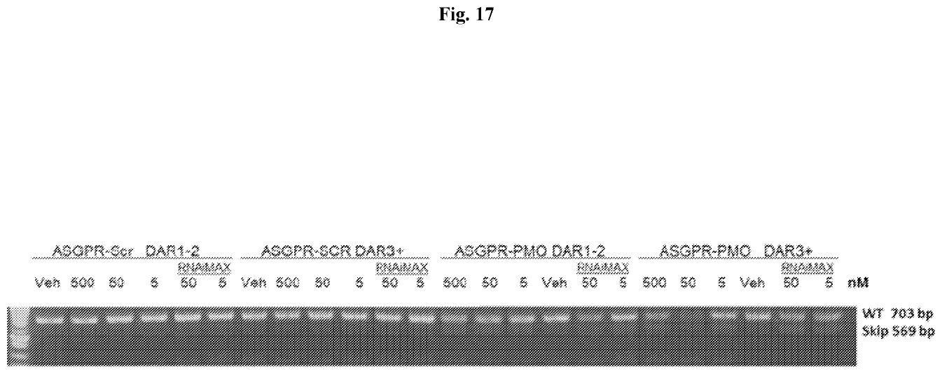

The ASGPR mAb-PMO (5′ATCCTCTTTGGTAACCTCACCTCAC) (SEQ ID NO: 47) conjugate targeting exon 11 of mouse PAH was made and characterized as described in Example 3. The conjugate was assessed for its ability to mediate exon 11 skipping in the mouse PAH gene in vitro in primary mouse hepatocytes using PCR (forward primer 5′-CTAGTGCCCTTGTTTTCAGA-3′ (SEQ ID NO: 52) and reverse primer 5′-AGGATCTACCACTGATGGG) (SEQ ID NO: 53). In brief, the potency of ASGPR mAb-PAH PMO conjugate was compared to ASGPR mAb-scramble PMO at multiple concentrations, with the relevant vehicle control. RNAiMAX was also used to transfect the conjugates as positive controls. PCR reactions were analyzed in a 1% TAE agarose gel as seen in FIG. 17. As seen from the gel in FIG. 17, the ASGPR mAb-PMO conjugate produced measurable exon 11 skipping comparable to the RNAiMAX transfected controls. The wild type product had an expected size of 703 bp and the skipped PAH Δ11 of 569 bp.

### Example 11. In Vivo Activity of ASGPR mAb-PMO Conjugates

The ASGPR mAb-PMO (5′ATCCTCTTTGGTAACCTCACCTCAC) (SEQ ID NO: 47) conjugate targeting exon 11 of mouse PAH was made and characterized as described in Example 3. The conjugate (DAR1/2 and DAR3+) was assessed for its ability to mediate exon skipping in vivo in wild type CD-1 mice using methods as described in Example 2. In brief, mice were dosed via intravenous (iv) injection with the mAb, vehicle control and indicated ASCs at the doses indicated as seen in Table 17.

TABLE 17
In vivo study design

| | | | mAb-ASO | PMO | PMO:mAb | Harvest |
|---|---|---|---|---|---|---|
| | | | mAb dose | Dose | Ratio | Time |
| Group | Test Article | N | (mg/kg) | (mg/kg) | (mol/mol) | (Weeks) |
| 1 | ASGPR mAb-PMO, | 3 | 50 | 5 | 1.5 | 1 |
| | DAR1/2 | | | | | |
| 2 | ASGPR mAb-PMO, | 3 | 50 | 5 | 1.5 | 2 |
| | DAR1/2 | | | | | |
| 3 | ASGPR mAb-PMO, | 3 | 50 | 5 | 1.5 | 4 |
| | DAR1/2 | | | | | |
| 4 | ASGPR mAb-PMO, | 2 | 50 | 10 | 3.0 | 1 |
| | DAR3+ | | | | | |
| 5 | ASGPR mAb-PMO, | 2 | 50 | 10 | 3.0 | 2 |
| | DAR3+ | | | | | |
| 6 | ASGPR mAb-PMO, | 2 | 50 | 10 | 3.0 | 4 |
| | DAR3+ | | | | | |
| 7 | ASGPR-Scr, DAR1/2 | 3 | 50 | | 1.5 | 2 |
| 8 | ASGPR-Scr, DAR3+ | 3 | 50 | | 3.0 | 2 |
| 9 | Vehicle | 3 | | | | 1 |
| 10 | Vehicle | 3 | | | | 2 |
| 11 | Vehicle | 3 | | | | 4 |

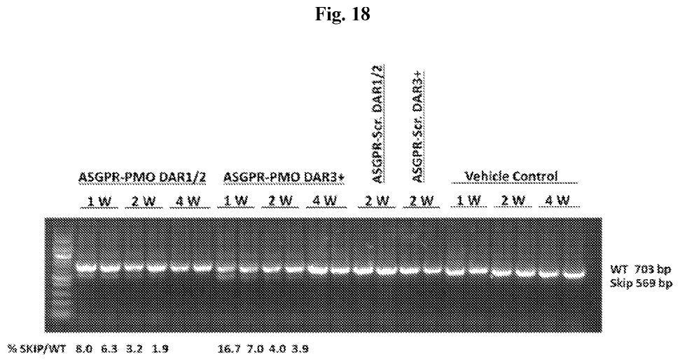

RNAs were isolated from harvested liver tissues and reverse transcribed. PCR reactions PG-3T using forward primer 5′-CTAGTGCCCTTGTTTTCAGA-3′ (SEQ ID NO: 52) and reverse primer 5′-AGGATCTACCACTGATGGG (SEQ ID NO: 53) were analyzed in a 1% TAE agarose gel as seen in FIG. 18. As can be seen from the gel in FIG. 18, the ASGPR mAb-PMO conjugates produced measurable exon 11 skipping in mouse livers up to two weeks. The wild type product had an expected size of 703 bp and the skipped PAH Δ11 of 569 bp.

### Example 12. Sequences

Table 18 illustrates exemplary target sequences to induce insertion, deletion, duplications, or alteration in the DMD gene using compositions and methods as described herein. Table 19 illustrates exemplary nucleotide sequences to induce an insertion, deletion, duplication, or alteration in the DMD gene using compositions and methods as described herein. Table 20 and Table 21 illustrate exemplary target sequences in several genes for inducing an insertion, deletion, duplications, or alteration in the gene. Table 22 illustrates exemplary sequences, including sequences in the DMD gene to induce an insertion, deletion, duplication, or alteration in the gene using compositions and methods as described herein.

TABLE 18
Exemplary target sequences to induce
insertion, deletion, duplications, or
alteration in the DMD gene

| | | SEQ |
|---|---|---|
| Target | | ID |
| Exon | Antisense Sequence | NO. |
| 19 | 5′ GCCUGAGCUGAUCUG | 54 |
| | CUGGCAUCUUGCAGUU 3′ | |
| 19 | 5′GCAGAAUUCGAUCCAC | 55 |
| or | CGGCUGUUCAAGCCUGAG | |
| 20 | CUGAUCUGCUCGCAUCUU | |
| | GCAGU3′ | |
| 20 | 5′ CAGCAGUAGUUGUCA | 56 |
| | UCUGCUC 3′ | |
| 21 | 5′ CACAAAGUCUGCAUC | 57 |
| | CAGGAACAUGGGUC 3′ | |
| 22 | 5′ CUGCAAUUCCCCGAG | 58 |
| | UCUCUGC 3′ | |
| 51 | 5′ CUCAUACCUUCUGCU | 59 |
| | UGAUGAUC 3′ | |
| 52 | 5′ UCCAACUGGGGACGC | 60 |
| | CUCUGUUCCAAAUCC 3′ | |

TABLE 19
Exemplary nucleotide sequences to induce an insertion, deletion,
duplication, or alteration in the DMD gene

| | | | SEQ |
|---|---|---|---|
| | | | ID |
| Gene | Target Location | Nucleotide Sequence (5′-3′) | NO. |
| DMD | H8A(−06+18) | GAUAGGUGGUAUCAACAUCUGUAA | 61 |
| DMD | H8A(−03+18) | GAUAGGUGGUAUCAACAUCUG | 62 |
| DMD | H8A(−07+18) | GAUAGGUGGUAUCAACAUCUGUAAG | 63 |
| DMD | H8A(−06+14) | GGUGGUAUCAACAUCUGUAA | 64 |
| DMD | H8A(−10+10) | GUAUCAACAUCUGUAAGCAC | 65 |
| DMD | H7A(+45+67) | UGCAUGUUCCAGUCGUUGUGUGG | 66 |
| DMD | H7A(+02+26) | CACUAUUCCAGUCAAAUAGGUCUGG | 67 |
| DMD | H7D(+15−10) | AUUUACCAACCUUCAGGAUCGAGUA | 68 |
| DMD | H7A(−18+03) | GGCCUAAAACACAUACACAUA | 69 |
| DMD | C6A(−10+10) | CAUUUUUGACCUACAUGUGG | 70 |
| DMD | C6A(−14+06) | UUUGACCUACAUGUGGAAAG | 71 |
| DMD | C6A(−14+12) | UACAUUUUUGACCUACAUGUGGAAAG | 72 |
| DMD | C6A(−13+09) | AUUUUUGACCUACAUGGGAAAG | 73 |
| DMD | CH6A(+69+91) | UACGAGUUGAUUGUCGGACCCAG | 74 |
| DMD | C6D(+12−13) | GUGGUCUCCUUACCUAUGACUGUGG | 75 |
| DMD | C6D(+06−11) | GGUCUCCUUACCUAUGA | 76 |
| DMD | H6D(+04−21) | UGUCUCAGUAAUCUUCUUACCUAU | 77 |
| DMD | H6D(+18−04) | UCUUACCUAUGACUAUGGAUGAGA | 78 |
| DMD | H4A(+13+32) | GCAUGAACUCUUGUGGAUCC | 79 |
| DMD | H4D(+04−16) | CCAGGGUACUACUUACAUUA | 80 |
| DMD | H4D(−24−44) | AUCGUGUGUCACAGCAUCCAG | 81 |
| DMD | H4A(+11+40) | UGUUCAGGGCAUGAACUCUUGUGGAUCCUU | 82 |
| DMD | H3A(+30+60) | UAGGAGGCGCCUCCCAUCCUGUAGGUCACUG | 83 |
| DMD | H3A(+35+65) | AGGUCUAGGAGGCGCCUCCCAUCCUGUAGGU | 84 |
| DMD | H3A(+30+54) | GCGCCUCCCAUCCUGUAGGUCACUG | 85 |
| DMD | H3D(+46−21) | CUUCGAGGAGGUCUAGGAGGCGCCUC | 86 |
| DMD | H3A(+30+50) | CUCCCAUCCUGUAGGUCACUG | 87 |
| DMD | H3D(+19−03) | UACCAGUUUUUGCCCUGUCAGG | 88 |
| DMD | H3A(−06+20) | UCAAUAUGCUGCUUCCCAAACUGAAA | 89 |
| DMD | H3A(+37+61) | CUAGGAGGCGCCUCCCAUCCUGUAG | 90 |
| DMD | H5A(+20+50) | UUAUGAUUUCCAUCUACGAUGUCAGUACUUC | 91 |
| DMD | H5D(+25−05) | CUUACCUGCCAGUGGAGGAUUAUAUUCCAAA | 92 |
| DMD | H5D(+10−15) | CAUCAGGAUUCUUACCUGCCAGUGG | 93 |
| DMD | H5A(+10+34) | CGAUGUCAGUACUUCCAAUAUUCAC | 94 |
| DMD | H5D(−04−21) | ACCAUUCAUCAGGAUUCU | 95 |
| DMD | H5D(+16−02) | ACCUGCCAGUGGAGGAUU | 96 |
| DMD | H5A(−07+20) | CCAAUAUUCACUAAAUCAACCUGUUAA | 97 |
| DMD | H5D(+18−12) | CAGGAUUGUUACCUGCCAGUGGAGGAUUAU | 98 |
| DMD | H5A(+05+35) | ACGAUGUCAGUACUUCCAAUAUUCACUAAAU | 99 |
| DMD | H5A(+15+45) | AUUUCCAUCUACGAUGUCAGUACUUCCAAUA | 100 |
| DMD | H10A(−05+16) | CAGGAGCUUCCAAAUGCUGCA | 101 |
| DMD | H10A(−05+24) | CUUGUCUUCAGGAGCUUCCAAAUGCUGCA | 102 |
| DMD | H10A(+98+119) | UCCUCAGCAGAAAGAAGCCACG | 103 |
| DMD | H10A(+130+149) | UUAGAAAUCUCUCCUUGUGC | 104 |
| DMD | H10A(−33−14) | UAAAUUGGGUGUUACACAAU | 105 |
| DMD | H11D(+26+49) | CCCUGAGGCAUUCCCAUCUUGAAU | 106 |
| DMD | H11D(+11−09) | AGGACUUACUUGCUUUGUUU | 107 |
| DMD | H11A(+118+140) | CUUGAAUUUAGGAGAUUCAUCUG | 108 |
| DMD | H11A(+75+97) | CAUCUUCUGAUAAUUUUCCUGUU | 109 |
| DMD | H12A(+52+75) | UCUUCUGUUUUUGUUAGCCAGUCA | 110 |
| DMD | H12A(−10+10) | UCUAUGUAAACUGAAAAUUU | 111 |
| DMD | H12A(+11+30) | UUCUGGAGAUCCAUUAAAAC | 112 |
| DMD | H13A(+77+100) | CAGCAGUUGCGUGAUCUCCACUAG | 113 |
| DMD | H13A(+55+75) | UUCAUCAACUACCACCACCAU | 114 |
| DMD | H13D(+06−19) | CUAAGCAAAAUAAUCUGACCUUAAG | 115 |
| DMD | H14A(+37+64) | CUUGUAAAAGAACCCAGCGGUCUUCUGU | 116 |
| DMD | H14A(+14+35) | CAUCUACAGAUGUUUGCCCAUC | 117 |
| DMD | H14A(+51+73) | GAAGGAUGUCUUGUAAAAGAACC | 118 |
| DMD | H14D(−02+18) | ACCUGUUCUUCAGUAAGACG | 119 |
| DMD | H14D(+14−10) | CAUGACACACCUGUUCUUCAGUAA | 120 |
| DMD | H14A(+61+80) | CAUUUGAGAAGGAUGUCUUG | 121 |
| DMD | H14A(−12+12) | AUCUCCCAAUACCUGGAGAAGAGA | 122 |
| DMD | H15A(−12+19) | GCCAUGCACUAAAAAGGCACUGCAAGACAUU | 123 |
| DMD | H15A(+48+71) | UCUUUAAAGCCAGUUGUGUGAAUC | 124 |
| DMD | H15A(+08+28) | UUUCUGAAAGCCAUGCACUAA | 125 |
| DMD | H15D(+17−08) | GUACAUACGGCCAGUUUUUGAAGAC | 126 |
| DMD | H16A(−12+19) | CUAGAUCCGCUUUUAAAACCUGUUAAAACAA | 127 |
| DMD | H16A(−06+25) | UCUUUUCUAGAUCCGCUUUUAAAACCUGUUA | 128 |
| DMD | H16A(−06+19) | CUAGAUCCGCUUUUAAAACCUGUUA | 129 |
| DMD | H16A(+87+109) | CCGUCUUCUGGGUCACUGACUUA | 130 |
| DMD | H16A(−07+19) | CUAGAUCCGCUUUUAAAACCUGUUAA | 131 |
| DMD | H16A(−07+13) | CCGCUUUUAAAACCUGUUAA | 132 |
| DMD | H16A(+12+37) | UGGAUUGCUUUUUCUUUUCUAGAUCC | 133 |
| DMD | H16A(+92+116) | CAUGCUUCCGUCUUCUGGGUCACUG | 134 |
| DMD | H16A(+45+67) | GAUCUUGUUUGAGUGAAUACAGU | 135 |
| DMD | H16A(+105+126) | GUUAUCCAGCCAUGCUUCCGUC | 136 |
| DMD | H16D(+05−20) | UGAUAAUUGGUAUCACUAACCUGUG | 137 |
| DMD | H16D(+12−11) | GUAUCACUAACCUGUGCUGUAC | 138 |
| DMD | H19A(+35+53) | CUGCUGGCAUCUUGCAGUU | 139 |
| DMD | H19A(+35+65) | GCCUGAGCUGAUCUGCUGGCAUCUUGCAGUU | 140 |
| DMD | H20A(+44+71) | CUGGCAGAAUUCGAUCCACCGGCUGUUC | 141 |
| DMD | H20A(+147+168) | CAGCAGUAGUUGUCAUCUGCUC | 142 |
| DMD | H20A(+185+203) | UGAUGGGGUGGUGGGUUGG | 143 |
| DMD | H20A(−08+17) | AUCUGCAUUAACACCCUCUAGAAAG | 144 |
| DMD | H20A(+30+53) | CCGGCUGUUCAGUUGUUCUGAGGC | 145 |
| DMD | H20A(−11+17) | AUCUGCAUUAACACCCUCUAGAAAGAAA | 146 |
| DMD | H20D(+08−20) | GAAGGAGAAGAGAUUCUUACCUUACAAA | 147 |
| DMD | H20A(+44+63) | AUUCGAUCCACCGGCUGUUC | 148 |
| DMD | H20A(+149+168 | CAGCAGUAGUUGUCAUCUGC | 149 |
| DMD | H21A(−06+16) | GCCGGUUGACUUCAUCCUGUGC | 150 |
| DMD | H21A(+85+106) | CUGCAUCCAGGAACAUGGGUCC | 151 |
| DMD | H21A(+85+108) | GUCUGCAUCCAGGAACAUGGGUC | 152 |
| DMD | H21A(+08+31) | GUUGAAGAUCUGAUAGCCGGUUGA | 153 |
| DMD | H21D(+18−07) | UACUUACUGUCUGUAGCUCUUUCU | 154 |
| DMD | H22A(+22+45) | CACUCAUGGUCUCCUGAUAGCGCA | 155 |
| DMD | H22A(+125+106) | CUGCAAUUCCCCGAGUCUCUGC | 156 |
| DMD | H22A(+47+69) | ACUGCUGGACCCAUGUCCUGAUG | 157 |
| DMD | H22A(+80+101) | CUAAGUUGAGGUAUGGAGAGU | 158 |
| DMD | H22D(+13−11) | UAUUCACAGACCUGCAAUUCCCC | 159 |
| DMD | H23A(+34+59) | ACAGUGGUGCUGAGAUAGUAUAGGCC | 160 |
| DMD | H23A(+18+39) | UAGGCCACUUUGUUGCUCUUGC | 161 |
| DMD | H23A(+72+90) | UUCAGAGGGCGCUUUCUUC | 162 |
| DMD | H24A(+48+70) | GGGCAGGCCAUUCCUCCUUCAGA | 163 |
| DMD | H24A(−02+22) | UCUUCAGGGUUUGUAUGUGAUUCU | 164 |
| DMD | H25A(+9+36) | CUGGGCUGAAUUGUCUGAAUAUCACUG | 165 |
| DMD | H25A(+131+156) | CUGUUGGCACAUGUGAUCCCACUGAG | 166 |
| DMD | H25D(+16−08) | GUCUAUACCUGUUGGCACAUGUGA | 167 |
| DMD | H26A(+132+156) | UGCUUUCUGUAAUUCAUCUGGAGUU | 168 |
| DMD | H26A(−07+19) | CCUCCUUUCUGGCAUAGACCUUCCAC | 169 |
| DMD | H26A(+68+92) | UGUGUCAUCCAUUCGUGCAUCUCUG | 170 |
| DMD | H27A(+82+106) | UUAAGGCCUCUUGUGCUACAGGUGG | 171 |
| DMD | H27A(−4+19) | GGGGCUCUUCUUUAGCUCUCUGA | 172 |
| DMD | H27D(+19−03) | GACUUCCAAAGUCUUGCAUUUC | 173 |
| DMD | H28A(−05+19) | GCCAACAUGCCCAAACUUCCUAAG | 174 |
| DMD | H28A(+99+124) | CAGAGAUUUCCUCAGCUCCGCCAGGA | 175 |
| DMD | H28D(+16−05) | CUUACAUCUAGCACCUCAGAG | 176 |
| DMD | H29A(+57+81) | UCCGCCAUCUGUUAGGGUCUGUGCC | 177 |
| DMD | H29A(+18+42) | AUUUGGGUUAUCCUCUGAAUGUCGC | 178 |
| DMD | H29D(+17−05) | CAUACCUCUUCAUGUAGUUCCC | 179 |
| DMD | H30A(+122+147) | CAUUUGAGCUGCGUCCACCUUGUCUG | 180 |
| DMD | H30A(+25+50) | UCCUGGGCAGACUGGAUGCUCUGUUC | 181 |
| DMD | H30D(+19−04) | UUGCCUGGGCUUCCUGAGGCAUU | 182 |
| DMD | H31D(+06−18) | UUCUGAAAUAACAUAUACCUGUGC | 183 |
| DMD | H31D(+03−22) | UAGUUUCUGAAAUAACAUAUACCUG | 184 |
| DMD | H31A(+05+25) | GACUUGUCAAAUCAGAUUGGA | 185 |
| DMD | H31D(+04−20) | GUUUCUGAAAUAACAUAUACCUGU | 186 |
| DMD | H32D(+04−16) | CACCAGAAAUACAUACCACA | 187 |
| DMD | H32A(+151+170) | CAAUGAUUUAGCUGUGACUG | 188 |
| DMD | H32A(+10+32) | CGAAACUUCAUGGAGACAUCUUG | 189 |
| DMD | H32A(+49+73) | CUUGUAGACGCUGCUCAAAAUUGGC | 190 |
| DMD | H33D(+09−11) | CAUGCACACACCUUUGCUCC | 191 |
| DMD | H33A(+53+76) | UCUGUACAAUCUGACGUCCAGUCU | 192 |
| DMD | H33A(+30+56) | GUCUUUAUCACCAUUUCCACUUCAGAC | 193 |
| DMD | H33A(+64+88) | CCGUCUGCUUUUUCUGUACAAUCUG | 194 |
| DMD | H34A(+83+104) | UCCAUAUCUGUAGCUGCCAGCC | 195 |
| DMD | H34A(+143+165) | CCAGGCAACUUCAGAAUCCAAAU | 196 |
| DMD | H34A(−20+10) | UUUCUGUUACCUGAAAAGAAUUAUAAUGAA | 197 |
| DMD | H34A(+46+70) | CAUUCAUUUCCUUUCGCAUCUUACG | 198 |
| DMD | H34A(+95+120) | UGAUCUCUUUGUCAAUUCCAUAUCUG | 199 |
| DMD | H34D(+10−20) | UUCAGUGAUAUAGGUUUUACCUUUCCCCAG | 200 |
| DMD | H34A(+72+96) | CUG UAG CUG CCA GCC AUU CUG UCA AG | 201 |
| DMD | H35A(+141+161) | UCU UCU GCU CGG GAG GUG ACA | 202 |
| DMD | H35A(+116+135) | CCA GUU ACU AUU CAG AAG AC | 203 |
| DMD | H35A(+24+43) | UCU UCA GGU GCA CCU UCU GU | 204 |
| DMD | H36A(+26+50) | UGUGAUGUGGUCCACAUUCUGGUCA | 205 |
| DMD | H36A(−02+18) | CCAUGUGUUUCUGGUAUUCC | 206 |
| DMD | H37A(+26+50) | CGUGUAGAGUCCACCUUUGGGCGUA | 207 |
| DMD | H37A(+82+105) | UACUAAUUUCCUGCAGUGGUCACC | 208 |
| DMD | H37A(+134+157) | UUCUGUGUGAAAUGGCUGCAAAUC | 209 |
| DMD | H38A(−01+19) | CCUUCAAAGGAAUGGAGGCC | 210 |
| DMD | H38A(+59+83) | UGCUGAAUUUCAGCCUCCAGUGGUU | 211 |
| DMD | H38A(+88+112) | UGAAGUCUUCCUCUUUCAGAUUCAC | 212 |
| DMD | H39A(+62+85) | CUGGCUUUCUCUCAUCUGUGAUUC | 213 |
| DMD | H39A(+39+58) | GUUGUAAGUUGUCUCCUCUU | 214 |
| DMD | H39A(+102+121) | UUGUCUGUAACAGCUGCUGU | 215 |
| DMD | H39D(+10−10) | GCUCUAAUACCUUGAGAGCA | 216 |
| DMD | H40A(−05+17) | CUUUGAGACCUCAAAUCCUGUU | 217 |
| DMD | H40A(+129+153) | CUUUAUUUUCCUUUCAUCUCUGGGC | 218 |
| DMD | H42A(−04+23) | AUCGUUUCUUCACGGACAGUGUGCUGG | 219 |
| DMD | H42A(+86+109) | GGGCUUGUGAGACAUGAGUGAUUU | 220 |
| DMD | H42D(+19−02) | ACCUUCAGAGGACUCCUCUUGC | 221 |
| DMD | H43D(+10−15) | UAUGUGUUACCUACCCUUGUCGGUC | 222 |
| DMD | H43A(+101+120) | GGAGAGAGCUUCCUGUAGCU | 223 |
| DMD | H43A(+78+100) | UCACCCUUUCCACAGGCGUUGCA | 224 |
| DMD | H44A(+85+104) | UUUGUGUCUUUCUGAGAAAC | 225 |
| DMD | H44D(+10−10) | AAAGACUUACCUUAAGAUAC | 226 |
| DMD | H44A(−06+14) | AUCUGUCAAAUCGCCUGCAG | 227 |
| DMD | H46D(+16−04) | UUACCUUGACUUGCUCAAGC | 228 |
| DMD | H46A(+90+109) | UCCAGGUUCAAGUGGGAUAC | 229 |
| DMD | H47A(+76+100) | GCUCUUCUGGGCUUAUGGGAGCACU | 230 |
| DMD | H47D(+25−02) | ACCUUUAUCCACUGGAGAUUUGUCUGC | 231 |
| DMD | H47A(−9+12) | UUCCACCAGUAACUGAAACAG | 232 |
| DMD | H50A(+02+30) | CCACUCAGAGCUCAGAUCUUCUAACUUCC | 233 |
| DMD | H50A(+07+33) | CUUCCACUCAGAGCUCAGAUCUUCUAA | 234 |
| DMD | H50D(+07−18) | GGGAUCCAGUAUACUUACAGGCUCC | 235 |
| DMD | H51A(−01+25) | ACCAGAGUAACAGUCUGAGUAGGAGC | 236 |
| DMD | H51D(+16−07) | CUCAUACCUUCUGCUUGAUGAUC | 237 |
| DMD | H51A(+111+134) | UUCUGUCCAAGCCCGGUUGAAAUC | 238 |
| DMD | H51A(+61+90) | ACAUCAAGGAAGAUGGCAUUUCUAGUUUGG | 239 |
| DMD | H51A(+66+90) | ACAUCAAGGAAGAUGGCAUUUCUAG | 240 |
| DMD | H51A(+66+95) | CUCCAACAUCAAGGAAGAUGGCAUUUCUAG | 241 |
| DMD | H51D(+08−17) | AUCAUUUUUUCUCAUACCUUCUGCU | 242 |
| DMD | H51A/D(+08−17) | AUCAUUUUUUCUCAUACCUUCUGCUAG | 243 |
| | &(−15+) | GAGCUAAAA | |
| DMD | H51A(+175+195) | CACCCACCAUCACCCUCUGUG | 245 |
| DMD | H51A(+199+220) | AUCAUCUCGUUGAUAUCCUCAA | 246 |
| DMD | H52A(−07+14) | UCCUGCAUUGUUGCCUGUAAG | 247 |
| DMD | H52A(+12+41) | UCCAACUGGGGACGCCUCUGUUCCAAAUCC | 248 |
| DMD | H52A(+17+37) | ACUGGGGACGCCUCUGUUCCA | 249 |
| DMD | H52A(+93+112) | CCGUAAUGAUUGUUCUAGCC | 250 |
| DMD | H52D(+05−15) | UGUUAAAAAACUUACUUCGA | 251 |
| DMD | H53A(+45+69) | CAUUCAACUGUUGCCUCCGGUUCUG | 252 |
| DMD | H53A(+39+62) | CUGUUGCCUCCGGUUCUGAAGGUG | 253 |
| DMD | H53A(+39+69) | CAUUCAACUGUUGCCUCCGGUUCUGAAGGUG | 254 |
| DMD | H53D(+14−07) | UACUAACCUUGGUUUCUGUGA | 255 |
| DMD | H53A(+23+47) | CUGAAGGUGUUCUUGUACUUCAUCC | 256 |
| DMD | H53A(+150+176) | UGUAUAGGGACCCUCCUUCCAUGACUC | 257 |
| DMD | H53D(+20−05) | CUAACCUUGGUUUCUGUGAUUUUCU | 258 |
| DMD | H53D(+09−18) | GGUAUCUUUGAUACUAACCUUGGUUUC | 259 |
| DMD | H53A(−12+10) | AUUCUUUCAACUAGAAUAAAAG | 260 |
| DMD | H53A(−07+18) | GAUUCUGAAUUCUUUCAACUAGAAU | 261 |
| DMD | H53A(+07+26) | AUCCCACUGAUUCUGAAUUC | 262 |
| DMD | H53A(+124+145) | UUGGCUCUGGCCUGUCCUAAGA | 263 |
| DMD | H46A(+86+115) | CUCUUUUCCAGGUUCAAGUGGGAUACUAGC | 264 |
| DMD | H46A(+107+137) | CAAGCUUUUCUUUUAGUUGCUGCUCUUUUCC | 265 |
| DMD | H46A(−10+20) | UAUUCUUUUGUUCUUCUAGCCUGGAGAAAG | 266 |
| DMD | H46A(+50+77) | CUGCUUCCUCCAACCAUAAAACAAAUUC | 267 |
| DMD | H45A(−06+20) | CCAAUGCCAUCCUGGAGUUCCUGUAA | 268 |
| DMD | H45A(+91+110) | UCCUGUAGAAUACUGGCAUC | 269 |
| DMD | H45A(+125+151) | UGCAGACCUCCUGCCACCGCAGAUUCA | 270 |
| DMD | H45D(+16−04) | CUACCUCUUUUUUCUGUCUG | 271 |
| DMD | H45A(+71+90) | UGUUUUUGAGGAUUGCUGAA | 272 |

* The first letter designates the species (e.g. H: human, M: murine, C: canine).
“#” designates target DMD exon number.
“A/D” indicates acceptor or donor splice site at the beginning and end of the exon, respectively.
(x y) represents the annealing coordinates where “-” or “+” indicate intronic or exonic sequences respectively.

TABLE 20
illustrate exemplary target sequences in
several genes for inducing an insertion,
deletion, duplications, or alteration
in the gene

| | | SEQ |
|---|---|---|
| | | ID |
| Gene | Nucleotide Sequence (5′-3′) | NO. |
| Bcl-x | TGGTTCTTACCCAGCCGCCG | 273 |
| β-globin 623 | GTTATTCTTTAGAATGGTGC | 274 |
| β-globin 654 | TGCTATTACCTTAACCCAGA | 275 |
| c-myc | CTGTGCTTACCGGGTTTTCCACCTCCC | 276 |
| c-myc | ATCGTCGTGACTGTCTGTTGGAGGG | 277 |
| c-myc | GCTCACGTTGAGGGGCATCG | 278 |
| c-myc | ACGTTGAGGGGCATCGTCGC | 279 |
| c-myc | GGGGCAUCGUCGUGACUGU/ | 280 |
| | CUGUUGGAGGG | |
| c-myc | CGUCGUGACUGUCUGUUGGAGG | 281 |
| c-myc | CGTCGTGACTGTCTGTTGGAGG | 282 |
| c-myc | GGCAUCGUCGCGGGAGGCUGCUGGAGCG | 283 |
| c-myc | CCGCGACAUAGGACGGAGAGCAGAGCCC | 284 |
| c-myc | ACTGTGAGGGCGATCGCTGC | 285 |
| c-myc | ACGATGAGTGGCATAGTCGC | 286 |
| c-myc | GGCATCGTCGCGGGAGGCTG | 287 |
| c-myc | GGGCATCGTCGCGGGAGGCT | 288 |
| c-myc | GGGGCATCGTCGCGGGAGGC | 289 |
| c-myc | AGGGGCATCGTCGCGGGAGG | 290 |
| c-myc | GAGGGGCATCGTCGCGGGAG | 291 |
| c-myc | TGAGGGGCATCGTCGCGGGA | 292 |
| c-myc | TTGAGGGGCATCGTCGCGGG | 293 |
| c-myc | GTTGAGGGGCATCGTCGCGG | 294 |
| c-myc | CGTTGAGGGGCATCGTCGCG | 295 |
| c-myc | ACGTTGAGGGGCATCGTCGC | 296 |
| c-myc | AACGTTGAGGGGCATCGTCG | 297 |
| c-myc | TAACGTTGAGGGGCATCGTC | 298 |
| c-myc | CTAACGTTGAGGGGCATCGT | 299 |
| c-myc | GCTAACGTTGAGGGGCATCG | 300 |
| c-myc | AGCTAACGTTGAGGGGCATC | 301 |
| c-myc | AAGCTAACGTTGAGGGGCAT | 302 |
| c-myc | GAAGCTAACGTTGAGGGGCA | 303 |
| BCL-2 (rat) | CTCCGCAATGCTGAAAGGTG | 304 |
| PCNA-1 (rat) | GGCGUGCCUCAAACAUGGUGGCGG | 305 |

TABLE 21
illustrate exemplary target sequences in
several genes for inducing an insertion,
deletion, duplications, or alteration
in the gene

| | | | SEQ |
|---|---|---|---|
| | Target | | ID |
| Gene | Location | Nucleotide Sequence (5′-3′) | NO. |
| Rat c-myc | 2553-79 | CTGTGCTTACCGGGTTTTCCACCTCCC | 306 |
| Rat c-myc | 4140-64 | ATCGTCGTGACTGTCTGTTGGAGGG | 307 |
| Rat c-myc | 4161-80 | GCTCACGTTGAGGGGCATCG | 308 |
| Rat CYP3A2 | 1155-74 | GGTCACTCACCGGTAGAGAA | 309 |
| Rat CYP3A2 | 1526-45 | GGGTTCCAAGTCTATAAAGG | 310 |
| Human | 31-44 | TGTGTCTTTTCCAG | 311 |
| androgen | | | |
| receptor exon 2 | | | |
| Human | 45-67 | TTTGGAGACTGCCAGGGACCATG | 312 |
| androgen | | | |
| receptor exon 2 | | | |
| Human | 48-67 | CATGGTCCCTGGCAGTCTCC | 313 |
| androgen | | | |
| receptor exon 2 | | | |
| Human | 45-80 | TCAATGGGCAAAACATGGTCCCT | 314 |
| androgen | | GGCAGTCTCCAAA | |
| receptor exon 2 | | | |
| Human | 28-43 | TTTGTGTTCTCCCAG | 315 |
| androgen | | | |
| receptor exon 3 | | | |
| Human | 44-66 | GGAAACAGAAGTACCTGTGCGCC | 316 |
| androgen | | | |
| receptor exon 3 | | | |
| Human | 49-66 | GGCGCACAGGTACTTCTG | 317 |
| androgen | | | |
| receptor exon 3 | | | |
| Human | 44-79 | AATCATTTCTGCTGGCGCAC | 318 |
| androgen | | AGGTACTTCTGTTTCC | |
| receptor exon 3 | | | |
| Human HCG-β | 1321-38 | CCCCTGCAGCACGCGGGT | 319 |
| subunit | | | |
| Human HCG-β | 1321-57 | GAGGCAGGGCCGGCAGGACC | 320 |
| subunit | | CCCTGCAGCACGCGGGT | |
| Human c-myc | 4506-25 | GGCATCGTCGCGGGAGGCTG | 321 |
| Human c-myc | 4507-26 | GGGCATCGTCGCGGGAGGCT | 322 |
| Human c-myc | 4508-27 | GGGGCATCGTCGCGGGAGGC | 323 |
| Human c-myc | 4509-28 | AGGGGCATCGTCGCGGGAGG | 324 |
| Human c-myc | 4510-29 | GAGGGGCATCGTCGCGGGAG | 325 |
| Human c-myc | 4511-30 | TGAGGGGCATCGTCGCGGGA | 326 |
| Human c-myc | 4512-31 | TTGAGGGGCATCGTCGCGGG | 327 |
| Human c-myc | 4513-32 | GTTGAGGGGCATCGTCGCGG | 328 |
| Human c-myc | 4514-33 | CGTTGAGGGGCATCGTCGCG | 329 |
| Human c-myc | 4515-34 | ACGTTGAGGGGCATCGTCGC | 330 |
| Human c-myc | 4516-35 | AACGTTGAGGGGCATCGTCG | 331 |
| Human c-myc | 4517-36 | TAACGTTGAGGGGCATCGTC | 332 |
| Human c-myc | 4518-37 | CTAACGTTGAGGGGCATCGT | 333 |
| Human c-myc | 4519-38 | GCTAACGTTGAGGGGCATCG | 334 |
| Human c-myc | 4520-39 | AGCTAACGTTGAGGGGCATC | 335 |
| Human c-myc | 4521-40 | AAGCTAACGTTGAGGGGCAT | 336 |
| Human c-myc | 4522-41 | GAAGCTAACGTTGAGGGGCA | 337 |
| Human c-myc | 6656-75 | TCCTCATCTTCTTGTTCCTC | 338 |
| Human c-myc | 6656-91 | AACAACATCGATTTCTTCCT | 339 |
| | | CATCTTCTTGTTCCTC | |
| Human p53 | 11691-708 | CCCGGAAGGCAGTCTGGC | 340 |
| Human p53 | 11689-724 | TCCTCCATGGCAGTGACCCG | 341 |
| | | GAAGGCAGTCTGGCTG | |
| Human | 376-94 | CTACTGGCCGCTGAAGGGC | 342 |
| abl (ds | | | |
| of bcr-abl | | | |
| fusion | | | |
| point) | | | |
| Human | 374-409 | GCTCAAAGTCAGATGCTAC | 343 |
| abl (ds | | TGGCCGCTGAAGGGCTT | |
| of bcr-abl | | | |
| fusion | | | |
| point) | | | |
| HW-1 rev | 5517-43 | TCGTCGGTCTCTCCGCTTCT | 344 |
| | | TCTTGCC | |
| HW-1 rev | 7885-7904 | CTCTGGTGGTGGGTAAGGGT | 345 |
| HW-1 rev | 7885-7921 | CGGGTCTGTCGGGTTCCCTC | 346 |
| | | TGGTGGTGGGTAAGGGT | |
| Rat c-myc | 4140-69 | GGGGCAUCGUCGUGACUGUC | 347 |
| | | UGUUGGAGGG | |
| Rat c-myc | 4141-62 | CGUCGUGACUGUCUGUUGGAGG | 348 |
| Rat c-myc | 4141-62 | CGTCGTGACTGTCTGTTGGAGG | 349 |
| Human c-myc | 4498-4505 | GGCAUCGUCGCGGGAGGCUG/ | 350 |
| | | CUGGAGCG | |
| Rat c-myc | 4364-91 | CCGCGACAUAGGACGGAGAG | 351 |
| | | CAGAGCCC | |

TABLE 22
Exemplary sequences, including sequences in the DMD gene to induce an
insertion, deletion, duplication, or alteration in the gene

| | | SEQ ID |
|---|---|---|
| Target | Nucleotide Sequence (5′-3′) | NO. |
| Hu.DMD.Exon44.25.001 | CTGCAGGTAAAAGCATATGGATCAA | 352 |
| Hu.DMD.Exon44.25.002 | ATCGCCTGCAGGTAAAAGCATATGG | 353 |
| Hu.DMD.Exon44.25.003 | GTCAAATCGCCTGCAGGTAAAAGCA | 354 |
| Hu.DMD.Exon44.25.004 | GATCTGTCAAATCGCCTGCAGGTAA | 355 |
| Hu.DMD.Exon44.25.005 | CAACAGATCTGTCAAATCGCCTGCA | 356 |
| Hu.DMD.Exon44.25.006 | TTTCTCAACAGATCTGTCAAATCGC | 357 |
| Hu.DMD.Exon44.25.007 | CCATTTCTCAACAGATCTGTCAAAT | 358 |
| Hu.DMD.Exon44.25.008 | ATAATGAAAACGCCGCCATTTCTCA | 359 |
| Hu.DMD.Exon44.25.009 | AAATATCTTTATATCATAATGAAAA | 360 |
| Hu.DMD.Exon44.25.010 | TGTTAGCCACTGATTAAATATCTTT | 361 |
| Hu.DMD.Exon44.25.011 | AAACTGTTCAGCTTCTGTTAGCCAC | 362 |
| Hu.DMD.Exon44.25.012 | TTGTGTCTTTCTGAGAAACTGTTCA | 363 |
| Hu.DMD.Exon44.25.013 | CCAATTCTCAGGAATTTGTGTCTTT | 364 |
| Hu.DMD.Exon44.25.014 | GTATTTAGCATGTTCCCAATTCTCA | 365 |
| Hu.DMD.Exon44.25.015 | CTTAAGATACCATTTGTATTTAGCA | 366 |
| Hu.DMD.Exon44.25.016 | CTTACCTTAAGATACCATTTGTATT | 367 |
| Hu.DMD.Exon44.25.017 | AAAGACTTACCTTAAGATACCATTT | 368 |
| Hu.DMD.Exon44.25.018 | AAATCAAAGACTTACCTTAAGATAC | 369 |
| Hu.DMD.Exon44.25.019 | AAAACAAATCAAAGACTTACCTTAA | 370 |
| Hu.DMD.Exon44.25.020 | TCGAAAAAACAAATCAAAGACTTAC | 371 |
| Hu.DMD.Exon45.25.001 | CTGTAAGATACCAAAAAGGCAAAAC | 372 |
| Hu.DMD.Exon45.25.002 | CCTGTAAGATACCAAAAAGGCAAAA | 373 |
| Hu.DMD.Exon45.25.002.2 | AGTTCCTGTAAGATACCAAAAAGGC | 374 |
| Hu.DMD.Exon45.25.003 | GAGTTCCTGTAAGATACCAAAAAGG | 375 |
| Hu.DMD.Exon45.25.003.2 | CCTGGAGTTCCTGTAAGATACCAAA | 376 |
| Hu.DMD.Exon45.25.004 | TCCTGGAGTTCCTGTAAGATACCAA | 377 |
| Hu.DMD.Exon45.25.004.2 | GCCATCCTGGAGTTCCTGTAAGATA | 378 |
| Hu.DMD.Exon45.25.005 | TGCCATCCTGGAGTTCCTGTAAGAT | 379 |
| Hu.DMD.Exon45.25.005.2 | CCAATGCCATCCTGGAGTTCCTGTA | 380 |
| Hu.DMD.Exon45.25.006 | CCCAATGCCATCCTGGAGTTCCTGT | 381 |
| Hu.DMD.Exon45.25.006.2 | GCTGCCCAATGCCATCCTGGAGTTC | 382 |
| Hu.DMD.Exon45.25.007 | CGCTGCCCAATGCCATCCTGGAGTT | 383 |
| Hu.DMD.Exon45.25.008 | AACAGTTTGCCGCTGCCCAATGCCA | 384 |
| Hu.DMD.Exon45.25.008.2 | CTGACAACAGTTTGCCGCTGCCCAA | 385 |
| Hu.DMD.Exon45.25.009 | GTTGCATTCAATGTTCTGACAACAG | 386 |
| Hu.DMD.Exon45.25.010 | GCTGAATTATTTCTTCCCCAGTTGC | 387 |
| Hu.DMD.Exon45.25.010.2 | ATTATTTCTTCCCCAGTTGCATTCA | 388 |
| Hu.DMD.Exon45.25.011 | GGCATCTGTTTTTGAGGATTGCTGA | 389 |
| Hu.DMD.Exon45.25.011.2 | TTTGAGGATTGCTGAATTATTTCTT | 390 |
| Hu.DMD.Exon45.25.012 | AATTTTTCCTGTAGAATACTGGCAT | 391 |
| Hu.DMD.Exon45.25.012.2 | ATACTGGCATCTGTTTTTGAGGATT | 392 |
| Hu.DMD.Exon45.25.013 | ACCGCAGATTCAGGCTTCCCAATTT | 393 |
| Hu.DMD.Exon45.25.013.2 | AATTTTTCCTGTAGAATACTGGCAT | 394 |
| Hu.DMD.Exon45.25.014 | CTGTTTGCAGACCTCCTGCCACCGC | 395 |
| Hu.DMD.Exon45.25.014.2 | AGATTCAGGCTTCCCAATTTTTCCT | 396 |
| Hu.DMD.Exon45.25.015 | CTCTTTTTTCTGTCTGACAGCTGTT | 397 |
| Hu.DMD.Exon45.25.015.2 | ACCTCCTGCCACCGCAGATTCAGGC | 398 |
| Hu.DMD.Exon45.25.016 | CCTACCTCTTTTTTCTGTCTGACAG | 399 |
| Hu.DMD.Exon45.25.016.2 | GACAGCTGTTTGCAGACCTCCTGCC | 400 |
| Hu.DMD.Exon45.25.017 | GTCGCCCTACCTCTTTTTTCTGTCT | 401 |
| Hu.DMD.Exon45.25.018 | GATCTGTCGCCCTACCTCTTTTTTC | 402 |
| Hu.DMD.Exon45.25.019 | TATTAGATCTGTCGCCCTACCTCTT | 403 |
| Hu.DMD.Exon45.25.020 | ATTCCTATTAGATCTGTCGCCCTAC | 404 |
| Hu.DMD.Exon45.20.001 | AGATACCAAAAAGGCAAAAC | 405 |
| Hu.DMD.Exon45.20.002 | AAGATACCAAAAAGGCAAAA | 406 |
| Hu.DMD.Exon45.20.003 | CCTGTAAGATACCAAAAAGG | 407 |
| Hu.DMD.Exon45.20.004 | GAGTTCCTGTAAGATACCAA | 408 |
| Hu.DMD.Exon45.20.005 | TCCTGGAGTTCCTGTAAGAT | 409 |
| Hu.DMD.Exon45.20.006 | TGCCATCCTGGAGTTCCTGT | 410 |
| Hu.DMD.Exon45.20.007 | CCCAATGCCATCCTGGAGTT | 411 |
| Hu.DMD.Exon45.20.008 | CGCTGCCCAATGCCATCCTG | 412 |
| Hu.DMD.Exon45.20.009 | CTGACAACAGTTTGCCGCTG | 413 |
| Hu.DMD.Exon45.20.010 | GTTGCATTCAATGTTCTGAC | 414 |
| Hu.DMD.Exon45.20.011 | ATTATTTCTTCCCCAGTTGC | 415 |
| Hu.DMD.Exon45.20.012 | TTTGAGGATTGCTGAATTAT | 416 |
| Hu.DMD.Exon45.20.013 | ATACTGGCATCTGTTTTTGA | 417 |
| Hu.DMD.Exon45.20.014 | AATTTTTCCTGTAGAATACT | 418 |
| Hu.DMD.Exon45.20.015 | AGATTCAGGCTTCCCAATTT | 419 |
| Hu.DMD.Exon45.20.016 | ACCTCCTGCCACCGCAGATT | 420 |
| Hu.DMD.Exon45.20.017 | GACAGCTGTTTGCAGACCTC | 421 |
| Hu.DMD.Exon45.20.018 | CTCTTTTTTCTGTCTGACAG | 422 |
| Hu.DMD.Exon45.20.019 | CCTACCTCTTTTTTCTGTCT | 423 |
| Hu.DMD.Exon45.20.020 | GTCGCCCTACCTCTTTTTTC | 424 |
| Hu.DMD.Exon45.20.021 | GATCTGTCGCCCTACCTCTT | 425 |
| Hu.DMD.Exon45.20.022 | TATTAGATCTGTCGCCCTAC | 426 |
| Hu.DMD.Exon45.20.023 | ATTCCTATTAGATCTGTCGC | 427 |
| Hu.DMD.Exon46.25.001 | GGGGGATTTGAGAAAATAAAATTAC | 428 |
| Hu.DMD.Exon46.25.002 | ATTTGAGAAAATAAAATTACCTTGA | 429 |
| Hu.DMD.Exon46.25.002.2 | CTAGCCTGGAGAAAGAAGAATAAAA | 430 |
| Hu.DMD.Exon46.25.003 | AGAAAATAAAATTACCTTGACTTGC | 431 |
| Hu.DMD.Exon46.25.003.2 | TTCTTCTAGCCTGGAGAAAGAAGAA | 432 |
| Hu.DMD.Exon46.25.004 | ATAAAATTACCTTGACTTGCTCAAG | 433 |
| Hu.DMD.Exon46.25.004.2 | TTTTGTTCTTCTAGCCTGGAGAAAG | 434 |
| Hu.DMD.Exon46.25.005 | ATTACCTTGACTTGCTCAAGCTTTT | 435 |
| Hu.DMD.Exon46.25.005.2 | TATTCTTTTGTTCTTCTAGCCTGGA | 436 |
| Hu.DMD.Exon46.25.006 | CTTGACTTGCTCAAGCTTTTCTTTT | 437 |
| Hu.DMD.Exon46.25.006.2 | CAAGATATTCTTTTGTTCTTCTAGC | 438 |
| Hu.DMD.Exon46.25.007 | CTTTTAGTTGCTGCTCTTTTCCAGG | 439 |
| Hu.DMD.Exon46.25.008 | CCAGGTTCAAGTGGGATACTAGCAA | 440 |
| Hu.DMD.Exon46.25.008.2 | ATCTCTTTGAAATTCTGACAAGATA | 441 |
| Hu.DMD.Exon46.25.009 | AGCAATGTTATCTGCTTCCTCCAAC | 442 |
| Hu.DMD.Exon46.25.009.2 | AACAAATTCATTTAAATCTCTTTGA | 443 |
| Hu.DMD.Exon46.25.010 | CCAACCATAAAACAAATTCATTTAA | 444 |
| Hu.DMD.Exon46.25.010.2 | TTCCTCCAACCATAAAACAAATTCA | 445 |
| Hu.DMD.Exon46.25.011 | TTTAAATCTCTTTGAAATTCTGACA | 446 |
| Hu.DMD.Exon46.25.012 | TGACAAGATATTCTTTTGTTCTTCT | 447 |
| Hu.DMD.Exon46.25.012.2 | TTCAAGTGGGATACTAGCAATGTTA | 448 |
| Hu.DMD.Exon46.25.013 | AGATATTCTTTTGTTCTTCTAGCCT | 449 |
| Hu.DMD.Exon46.25.013.2 | CTGCTCTTTTCCAGGTTCAAGTGGG | 450 |
| Hu.DMD.Exon46.25.014 | TTCTTTTGTTCTTCTAGCCTGGAGA | 451 |
| Hu.DMD.Exon46.25.014.2 | CTTTTCTTTTAGTTGCTGCTCTTTT | 452 |
| Hu.DMD.Exon46.25.015 | TTGTTCTTCTAGCCTGGAGAAAGAA | 453 |
| Hu.DMD.Exon46.25.016 | CTTCTAGCCTGGAGAAAGAAGAATA | 454 |
| Hu.DMD.Exon46.25.017 | AGCCTGGAGAAAGAAGAATAAAATT | 455 |
| Hu.DMD.Exon46.25.018 | CTGGAGAAAGAAGAATAAAATTGTT | 456 |
| Hu.DMD.Exon46.20.001 | GAAAGAAGAATAAAATTGTT | 457 |
| Hu.DMD.Exon46.20.002 | GGAGAAAGAAGAATAAAATT | 458 |
| Hu.DMD.Exon46.20.003 | AGCCTGGAGAAAGAAGAATA | 459 |
| Hu.DMD.Exon46.20.004 | CTTCTAGCCTGGAGAAAGAA | 460 |
| Hu.DMD.Exon46.20.005 | TTGTTCTTCTAGCCTGGAGA | 461 |
| Hu.DMD.Exon46.20.006 | TTCTTTTGTTCTTCTAGCCT | 462 |
| Hu.DMD.Exon46.20.007 | TGACAAGATATTCTTTTGTT | 463 |
| Hu.DMD.Exon46.20.008 | ATCTCTTTGAAATTCTGACA | 464 |
| Hu.DMD.Exon46.20.009 | AACAAATTCATTTAAATCTC | 465 |
| Hu.DMD.Exon46.20.010 | TTCCTCCAACCATAAAACAA | 466 |
| Hu.DMD.Exon46.20.011 | AGCAATGTTATCTGCTTCCT | 467 |
| Hu.DMD.Exon46.20.012 | TTCAAGTGGGATACTAGCAA | 468 |
| Hu.DMD.Exon46.20.013 | CTGCTCTTTTCCAGGTTCAA | 469 |
| Hu.DMD.Exon46.20.014 | CTTTTCTTTTAGTTGCTGCT | 470 |
| Hu.DMD.Exon46.20.015 | CTTGACTTGCTCAAGCTTTT | 471 |
| Hu.DMD.Exon46.20.016 | ATTACCTTGACTTGCTCAAG | 472 |
| Hu.DMD.Exon46.20.017 | ATAAAATTACCTTGACTTGC | 473 |
| Hu.DMD.Exon46.20.018 | AGAAAATAAAATTACCTTGA | 474 |
| Hu.DMD.Exon46.20.019 | ATTTGAGAAAATAAAATTAC | 475 |
| Hu.DMD.Exon46.20.020 | GGGGGATTTGAGAAAATAAA | 476 |
| Hu.DMD.Exon47.25.001 | CTGAAACAGACAAATGCAACAACGT | 477 |
| Hu.DMD.Exon47.25.002 | AGTAACTGAAACAGACAAATGCAAC | 478 |
| Hu.DMD.Exon47.25.003 | CCACCAGTAACTGAAACAGACAAAT | 479 |
| Hu.DMD.Exon47.25.004 | CTCTTCCACCAGTAACTGAAACAGA | 480 |
| Hu.DMD.Exon47.25.005 | GGCAACTCTTCCACCAGTAACTGAA | 481 |
| Hu.DMD.Exon47.25.006 | GCAGGGGCAACTCTTCCACCAGTAA | 482 |
| Hu.DMD.Exon47.25.007 | CTGGCGCAGGGGCAACTCTTCCACC | 483 |
| Hu.DMD.Exon47.25.008 | TTTAATTGTTTGAGAATTCCCTGGC | 484 |
| Hu.DMD.Exon47.25.008.2 | TTGTTTGAGAATTCCCTGGCGCAGG | 485 |
| Hu.DMD.Exon47.25.009 | GCACGGGTCCTCCAGTTTCATTTAA | 486 |
| Hu.DMD.Exon47.25.009.2 | TCCAGTTTCATTTAATTGTTTGAGA | 487 |
| Hu.DMD.Exon47.25.010 | GCTTATGGGAGCACTTACAAGCACG | 488 |
| Hu.DMD.Exon47.25.010.2 | TACAAGCACGGGTCCTCCAGTTTCA | 489 |
| Hu.DMD.Exon47.25.011 | AGTTTATCTTGCTCTTCTGGGCTTA | 490 |
| Hu.DMD.Exon47.25.012 | TCTGCTTGAGCTTATTTTCAAGTTT | 491 |
| Hu.DMD.Exon47.25.012.2 | ATCTTGCTCTTCTGGGCTTATGGGA | 492 |
| Hu.DMD.Exon47.25.013 | CTTTATCCACTGGAGATTTGTCTGC | 493 |
| Hu.DMD.Exon47.25.013.2 | CTTATTTTCAAGTTTATCTTGCTCT | 494 |
| Hu.DMD.Exon47.25.014 | CTAACCTTTATCCACTGGAGATTTG | 495 |
| Hu.DMD.Exon47.25.014.2 | ATTTGTCTGCTTGAGCTTATTTTCA | 496 |
| Hu.DMD.Exon47.25.015 | AATGTCTAACCTTTATCCACTGGAG | 497 |
| Hu.DMD.Exon47.25.016 | TGGTTAATGTCTAACCTTTATCCAC | 498 |
| Hu.DMD.Exon47.25.017 | AGAGATGGTTAATGTCTAACCTTTA | 499 |
| Hu.DMD.Exon47.25.018 | ACGGAAGAGATGGTTAATGTCTAAC | 500 |
| Hu.DMD.Exon47.20.001 | ACAGACAAATGCAACAACGT | 501 |
| Hu.DMD.Exon47.20.002 | CTGAAACAGACAAATGCAAC | 502 |
| Hu.DMD.Exon47.20.003 | AGTAACTGAAACAGACAAAT | 503 |
| Hu.DMD.Exon47.20.004 | CCACCAGTAACTGAAACAGA | 504 |
| Hu.DMD.Exon47.20.005 | CTCTTCCACCAGTAACTGAA | 505 |
| Hu.DMD.Exon47.20.006 | GGCAACTCTTCCACCAGTAA | 506 |
| Hu.DMD.Exon47.20.007 | CTGGCGCAGGGGCAACTCTT | 507 |
| Hu.DMD.Exon47.20.008 | TTGTTTGAGAATTCCCTGGC | 508 |
| Hu.DMD.Exon47.20.009 | TCCAGTTTCATTTAATTGTT | 509 |
| Hu.DMD.Exon47.20.010 | TACAAGCACGGGTCCTCCAG | 510 |
| Hu.DMD.Exon47.20.011 | GCTTATGGGAGCACTTACAA | 511 |
| Hu.DMD.Exon47.20.012 | ATCTTGCTCTTCTGGGCTTA | 512 |
| Hu.DMD.Exon47.20.013 | CTTATTTTCAAGTTTATCTT | 513 |
| Hu.DMD.Exon47.20.014 | ATTTGTCTGCTTGAGCTTAT | 514 |
| Hu.DMD.Exon47.20.015 | CTTTATCCACTGGAGATTTG | 515 |
| Hu.DMD.Exon47.20.016 | CTAACCTTTATCCACTGGAG | 516 |
| Hu.DMD.Exon47.20.017 | AATGTCTAACCTTTATCCAC | 517 |
| Hu.DMD.Exon47.20.018 | TGGTTAATGTCTAACCTTTA | 518 |
| Hu.DMD.Exon47.20.019 | AGAGATGGTTAATGTCTAAC | 519 |
| Hu.DMD.Exon47.20.020 | ACGGAAGAGATGGTTAATGT | 520 |
| Hu.DMD.Exon48.25.001 | CTGAAAGGAAAATACATTTTAAAAA | 521 |
| Hu.DMD.Exon48.25.002 | CCTGAAAGGAAAATACATTTTAAAA | 522 |
| Hu.DMD.Exon48.25.002.2 | GAAACCTGAAAGGAAAATACATTTT | 523 |
| Hu.DMD.Exon48.25.003 | GGAAACCTGAAAGGAAAATACATTT | 524 |
| Hu.DMD.Exon48.25.003.2 | CTCTGGAAACCTGAAAGGAAAATAC | 525 |
| Hu.DMD.Exon48.25.004 | GCTCTGGAAACCTGAAAGGAAAATA | 526 |
| Hu.DMD.Exon48.25.004.2 | TAAAGCTCTGGAAACCTGAAAGGAA | 527 |
| Hu.DMD.Exon48.25.005 | GTAAAGCTCTGGAAACCTGAAAGGA | 528 |
| Hu.DMD.Exon48.25.005.2 | TCAGGTAAAGCTCTGGAAACCTGAA | 529 |
| Hu.DMD.Exon48.25.006 | CTCAGGTAAAGCTCTGGAAACCTGA | 530 |
| Hu.DMD.Exon48.25.006.2 | GTTTCTCAGGTAAAGCTCTGGAAAC | 531 |
| Hu.DMD.Exon48.25.007 | TGTTTCTCAGGTAAAGCTCTGGAAA | 532 |
| Hu.DMD.Exon48.25.007.2 | AATTTCTCCTTGTTTCTCAGGTAAA | 533 |
| Hu.DMD.Exon48.25.008 | TTTGAGCTTCAATTTCTCCTTGTTT | 534 |
| Hu.DMD.Exon48.25.008 | TTTTATTTGAGCTTCAATTTCTCCT | 535 |
| Hu.DMD.Exon48.25.009 | AAGCTGCCCAAGGTCTTTTATTTGA | 536 |
| Hu.DMD.Exon48.25.010 | AGGTCTTCAAGCTTTTTTTCAAGCT | 537 |
| Hu.DMD.Exon48.25.010.2 | TTCAAGCTTTTTTTCAAGCTGCCCA | 538 |
| Hu.DMD.Exon48.25.011 | GATGATTTAACTGCTCTTCAAGGTC | 539 |
| Hu.DMD.Exon48.25.011.2 | CTGCTCTTCAAGGTCTTCAAGCTTT | 540 |
| Hu.DMD.Exon48.25.012 | AGGAGATAACCACAGCAGCAGATGA | 541 |
| Hu.DMD.Exon48.25.012.2 | CAGCAGATGATTTAACTGCTCTTCA | 542 |
| Hu.DMD.Exon48.25.013 | ATTTCCAACTGATTCCTAATAGGAG | 543 |
| Hu.DMD.Exon48.25.014 | CTTGGTTTGGTTGGTTATAAATTTC | 544 |
| Hu.DMD.Exon48.25.014.2 | CAACTGATTCCTAATAGGAGATAAC | 545 |
| Hu.DMD.Exon48.25.015 | CTTAACGTCAAATGGTCCTTCTTGG | 546 |
| Hu.DMD.Exon48.25.015.2 | TTGGTTATAAATTTCCAACTGATTC | 547 |
| Hu.DMD.Exon48.25.016 | CCTACCTTAACGTCAAATGGTCCTT | 548 |
| Hu.DMD.Exon48.25.016.2 | TCCTTCTTGGTTTGGTTGGTTATAA | 549 |
| Hu.DMD.Exon48.25.017 | AGTTCCCTACCTTAACGTCAAATGG | 550 |
| Hu.DMD.Exon48.25.018 | CAAAAAGTTCCCTACCTTAACGTCA | 551 |
| Hu.DMD.Exon48.25.019 | TAAAGCAAAAAGTTCCCTACCTTAA | 552 |
| Hu.DMD.Exon48.25.020 | ATATTTAAAGCAAAAAGTTCCCTAC | 553 |
| Hu.DMD.Exon48.20.001 | AGGAAAATACATTTTAAAAA | 554 |
| Hu.DMD.Exon48.20.002 | AAGGAAAATACATTTTAAAA | 555 |
| Hu.DMD.Exon48.20.003 | CCTGAAAGGAAAATACATTT | 556 |
| Hu.DMD.Exon48.20.004 | GGAAACCTGAAAGGAAAATA | 557 |
| Hu.DMD.Exon48.20.005 | GCTCTGGAAACCTGAAAGGA | 558 |
| Hu.DMD.Exon48.20.006 | GTAAAGCTCTGGAAACCTGA | 559 |
| Hu.DMD.Exon48.20.007 | CTCAGGTAAAGCTCTGGAAA | 560 |
| Hu.DMD.Exon48.20.008 | AATTTCTCCTTGTTTCTCAG | 561 |
| Hu.DMD.Exon48.20.009 | TTTTATTTGAGCTTCAATTT | 562 |
| Hu.DMD.Exon48.20.010 | AAGCTGCCCAAGGTCTTTTA | 563 |
| Hu.DMD.Exon48.20.011 | TTCAAGCTTTTTTTCAAGCT | 564 |
| Hu.DMD.Exon48.20.012 | CTGCTCTTCAAGGTCTTCAA | 565 |
| Hu.DMD.Exon48.20.013 | CAGCAGATGATTTAACTGCT | 566 |
| Hu.DMD.Exon48.20.014 | AGGAGATAACCACAGCAGCA | 567 |
| Hu.DMD.Exon48.20.015 | CAACTGATTCCTAATAGGAG | 568 |
| Hu.DMD.Exon48.20.016 | TTGGTTATAAATTTCCAACT | 569 |
| Hu.DMD.Exon48.20.017 | TCCTTCTTGGTTTGGTTGGT | 570 |
| Hu.DMD.Exon48.20.018 | CTTAACGTCAAATGGTCCTT | 571 |
| Hu.DMD.Exon48.20.019 | CCTACCTTAACGTCAAATGG | 572 |
| Hu.DMD.Exon48.20.020 | AGTTCCCTACCTTAACGTCA | 573 |
| Hu.DMD.Exon48.20.021 | CAAAAAGTTCCCTACCTTAA | 574 |
| Hu.DMD.Exon48.20.022 | TAAAGCAAAAAGTTCCCTAC | 575 |
| Hu.DMD.Exon48.20.023 | ATATTTAAAGCAAAAAGTTC | 576 |
| Hu.DMD.Exon49.25.001 | CTGGGGAAAAGAACCCATATAGTGC | 577 |
| Hu.DMD.Exon49.25.002 | TCCTGGGGAAAAGAACCCATATAGT | 578 |
| Hu.DMD.Exon49.25.002.2 | GTTTCCTGGGGAAAAGAACCCATAT | 579 |
| Hu.DMD.Exon49.25.003 | CAGTTTCCTGGGGAAAAGAACCCAT | 580 |
| Hu.DMD.Exon49.25.003.2 | TTTCAGTTTCCTGGGGAAAAGAACC | 581 |
| Hu.DMD.Exon49.25.004 | TATTTCAGTTTCCTGGGGAAAAGAA | 582 |
| Hu.DMD.Exon49.25.004.2 | TGCTATTTCAGTTTCCTGGGGAAAA | 583 |
| Hu.DMD.Exon49.25.005 | ACTGCTATTTCAGTTTCCTGGGGAA | 584 |
| Hu.DMD.Exon49.25.005.2 | TGAACTGCTATTTCAGTTTCCTGGG | 585 |
| Hu.DMD.Exon49.25.006 | CTTGAACTGCTATTTCAGTTTCCTG | 586 |
| Hu.DMD.Exon49.25.006.2 | TAGCTTGAACTGCTATTTCAGTTTC | 587 |
| Hu.DMD.Exon49.25.007 | TTTAGCTTGAACTGCTATTTCAGTT | 588 |
| Hu.DMD.Exon49.25.008 | TTCCACATCCGGTTGTTTAGCTTGA | 589 |
| Hu.DMD.Exon49.25.009 | TGCCCTTTAGACAAAATCTCTTCCA | 590 |
| Hu.DMD.Exon49.25.009.2 | TTTAGACAAAATCTCTTCCACATCC | 591 |
| Hu.DMD.Exon49.25.010 | GTTTTTCCTTGTACAAATGCTGCCC | 592 |
| Hu.DMD.Exon49.25.010.2 | GTACAAATGCTGCCCTTTAGACAAA | 593 |
| Hu.DMD.Exon49.25.011 | CTTCACTGGCTGAGTGGCTGGTTTT | 594 |
| Hu.DMD.Exon49.25.011.2 | GGCTGGTTTTTCCTTGTACAAATGC | 595 |
| Hu.DMD.Exon49.25.012 | ATTACCTTCACTGGCTGAGTGGCTG | 596 |
| Hu.DMD.Exon49.25.013 | GCTTCATTACCTTCACTGGCTGAGT | 597 |
| Hu.DMD.Exon49.25.014 | AGGTTGCTTCATTACCTTCACTGGC | 598 |
| Hu.DMD.Exon49.25.015 | GCTAGAGGTTGCTTCATTACCTTCA | 599 |
| Hu.DMD.Exon49.25.016 | ATATTGCTAGAGGTTGCTTCATTAC | 600 |
| Hu.DMD.Exon49.20.001 | GAAAAGAACCCATATAGTGC | 601 |
| Hu.DMD.Exon49.20.002 | GGGAAAAGAACCCATATAGT | 602 |
| Hu.DMD.Exon49.20.003 | TCCTGGGGAAAAGAACCCAT | 603 |
| Hu.DMD.Exon49.20.004 | CAGTTTCCTGGGGAAAAGAA | 604 |
| Hu.DMD.Exon49.20.005 | TATTTCAGTTTCCTGGGGAA | 605 |
| Hu.DMD.Exon49.20.006 | ACTGCTATTTCAGTTTCCTG | 606 |
| Hu.DMD.Exon49.20.007 | CTTGAACTGCTATTTCAGTT | 607 |
| Hu.DMD.Exon49.20.008 | TTTAGCTTGAACTGCTATTT | 608 |
| Hu.DMD.Exon49.20.009 | TTCCACATCCGGTTGTTTAG | 609 |
| Hu.DMD.Exon49.20.010 | TTTAGACAAAATCTCTTCCA | 610 |
| Hu.DMD.Exon49.20.011 | GTACAAATGCTGCCCTTTAG | 611 |
| Hu.DMD.Exon49.20.012 | GGCTGGTTTTTCCTTGTACA | 612 |
| Hu.DMD.Exon49.20.013 | CTTCACTGGCTGAGTGGCTG | 613 |
| Hu.DMD.Exon49.20.014 | ATTACCTTCACTGGCTGAGT | 614 |
| Hu.DMD.Exon49.20.015 | GCTTCATTACCTTCACTGGC | 615 |
| Hu.DMD.Exon49.20.016 | AGGTTGCTTCATTACCTTCA | 616 |
| Hu.DMD.Exon49.20.017 | GCTAGAGGTTGCTTCATTAC | 617 |
| Hu.DMD.Exon49.20.018 | ATATTGCTAGAGGTTGCTTC | 618 |
| Hu.DMD.Exon50.25.001 | CTTTAACAGAAAAGCATACACATTA | 619 |
| Hu.DMD.Exon50.25.002 | TCCTCTTTAACAGAAAAGCATACAC | 620 |
| Hu.DMD.Exon50.25.002.2 | TTCCTCTTTAACAGAAAAGCATACA | 621 |
| Hu.DMD.Exon50.25.003 | TAACTTCCTCTTTAACAGAAAAGCA | 622 |
| Hu.DMD.Exon50.25.003.2 | CTAACTTCCTCTTTAACAGAAAAGC | 623 |
| Hu.DMD.Exon50.25.004 | TCTTCTAACTTCCTCTTTAACAGAA | 624 |
| Hu.DMD.Exon50.25.004.2 | ATCTTCTAACTTCCTCTTTAACAGA | 625 |
| Hu.DMD.Exon50.25.005 | TCAGATCTTCTAACTTCCTCTTTAA | 626 |
| Hu.DMD.Exon50.25.005.2 | CTCAGATCTTCTAACTTCCTCTTTA | 627 |
| Hu.DMD.Exon50.25.006 | AGAGCTCAGATCTTCTAACTTCCTC | 628 |
| Hu.DMD.Exon50.25.006.2 | CAGAGCTCAGATCTTCTAACTTCCT | 629 |
| NG-08-0731 | | |
| Hu.DMD.Exon50.25.007 | CACTCAGAGCTCAGATCTTCTACT | 630 |
| Hu.DMD.Exon50.25.007.2 | CCTTCCACTCAGAGCTCAGATCTTC | 631 |
| Hu.DMD.Exon50.25.008 | GTAAACGGTTTACCGCCTTCCACTC | 632 |
| Hu.DMD.Exon50.25.009 | CTTTGCCCTCAGCTCTTGAAGTAAA | 633 |
| Hu.DMD.Exon50.25.009.2 | CCCTCAGCTCTTGAAGTAAACGGTT | 634 |
| Hu.DMD.Exon50.25.010 | CCAGGAGCTAGGTCAGGCTGCTTTG | 635 |
| Hu.DMD.Exon50.25.010.2 | GGTCAGGCTGCTTTGCCCTCAGCTC | 636 |
| Hu.DMD.Exon50.25.011 | AGGCTCCAATAGTGGTCAGTCCAGG | 637 |
| Hu.DMD.Exon50.25.011.2 | TCAGTCCAGGAGCTAGGTCAGGCTG | 638 |
| Hu.DMD.Exon50.25.012 | CTTACAGGCTCCAATAGTGGTCAGT | 639 |
| AVI-5038 | | |
| Hu.DMD.Exon50.25.013 | GTATACTTACAGGCTCCAATAGTGG | 640 |
| Hu.DMD.Exon50.25.014 | ATCCAGTATACTTACAGGCTCCAAT | 641 |
| Hu.DMD.Exon50.25.015 | ATGGGATCCAGTATACTTACAGGCT | 642 |
| NG-08-0741 | | |
| Hu.DMD.Exon50.25.016 | AGAGAATGGGATCCAGTATACTTAC | 643 |
| NG-08-0742 | | |
| Hu.DMD.Exon50.20.001 | ACAGAAAAGCATACACATTA | 644 |
| Hu.DMD.Exon50.20.002 | TTTAACAGAAAAGCATACAC | 645 |
| Hu.DMD.Exon50.20.003 | TCCTCTTTAACAGAAAAGCA | 646 |
| Hu.DMD.Exon50.20.004 | TAACTTCCTCTTTAACAGAA | 647 |
| Hu.DMD.Exon50.20.005 | TCTTCTAACTTCCTCTTTAA | 648 |
| Hu.DMD.Exon50.20.006 | TCAGATCTTCTAACTTCCTC | 649 |
| Hu.DMD.Exon50.20.007 | CCTTCCACTCAGAGCTCAGA | 650 |
| Hu.DMD.Exon50.20.008 | GTAAACGGTTTACCGCCTTC | 651 |
| Hu.DMD.Exon50.20.009 | CCCTCAGCTCTTGAAGTAAA | 652 |
| Hu.DMD.Exon50.20.010 | GGTCAGGCTGCTTTGCCCTC | 653 |
| Hu.DMD.Exon50.20.011 | TCAGTCCAGGAGCTAGGTCA | 654 |
| Hu.DMD.Exon50.20.012 | AGGCTCCAATAGTGGTCAGT | 655 |
| Hu.DMD.Exon50.20.013 | CTTACAGGCTCCAATAGTGG | 656 |
| Hu.DMD.Exon50.20.014 | GTATACTTACAGGCTCCAAT | 657 |
| Hu.DMD.Exon50.20.015 | ATCCAGTATACTTACAGGCT | 658 |
| Hu.DMD.Exon50.20.016 | ATGGGATCCAGTATACTTAC | 659 |
| Hu.DMD.Exon50.20.017 | AGAGAATGGGATCCAGTATA | 660 |
| Hu.DMD.Exon51.25.001- | CTAAAATATTTTGGGTTTTTGCAAAA | 661 |
| 44 | | |
| Hu.DMD.Exon51.25.002- | GCTAAAATATTTTGGGTTTTTGCAAA | 662 |
| 45 | | |
| Hu.DMD.Exon51.25.002.2- | TAGGAGCTAAAATATTTTGGGTTTTT | 663 |
| 46 | | |
| Hu.DMD.Exon51.25.003 | AGTAGGAGCTAAAATATTTTGGGTT | 664 |
| Hu.DMD.Exon51.25.003.2 | TGAGTAGGAGCTAAAATATTTTGGG | 665 |
| Hu.DMD.Exon51.25.004 | CTGAGTAGGAGCTAAAATATTTTGGG | 666 |
| Hu.DMD.Exon51.25.004.2 | CAGTCTGAGTAGGAGCTAAAATATT | 667 |
| Hu.DMD.Exon51.25.005 | ACAGTCTGAGTAGGAGCTAAAATATT | 668 |
| Hu.DMD.Exon51.25.005.2 | GAGTAACAGTCTGAGTAGGAGCTAAA | 669 |
| Hu.DMD.Exon51.25.006 | CAGAGTAACAGTCTGAGTAGGAGCT | 670 |
| Hu.DMD.Exon51.25.006.2 | CACCAGAGTAACAGTCTGAGTAGGAG | 671 |
| Hu.DMD.Exon51.25.007 | GTCACCAGAGTAACAGTCTGAGTAG | 672 |
| Hu.DMD.Exon51.25.007.2 | AACCACAGGTTGTGTCACCAGAGTAA | 673 |
| Hu.DMD.Exon51.25.008 | GTTGTGTCACCAGAGTAACAGTCTG | 674 |
| Hu.DMD.Exon51.25.009 | TGGCAGTTTCCTTAGTAACCACAGGT | 675 |
| Hu.DMD.Exon51.25.010 | ATTTCTAGTTTGGAGATGGCAGTTTC | 676 |
| Hu.DMD.Exon51.25.010.2 | GGAAGATGGCATTTCTAGTTTGGAG | 677 |
| Hu.DMD.Exon51.25.011 | CATCAAGGAAGATGGCATTTCTAGTT | 678 |
| Hu.DMD.Exon51.25.011.2 | GAGCAGGTACCTCCAACATCAAGGAA | 679 |
| Hu.DMD.Exon51.25.012 | ATCTGCCAGAGCAGGTACCTCCAAC | 680 |
| Hu.DMD.Exon51.25.013 | AAGTTCTGTCCAAGCCCGGTTGAAAT | 681 |
| Hu.DMD.Exon51.25.013.2 | CGGTTGAAATCTGCCAGAGCAGGTAC | 682 |
| Hu.DMD.Exon51.25.014 | GAGAAAGCCAGTCGGTAAGTTCTGTC | 683 |
| Hu.DMD.Exon51.25.014.2 | GTCGGTAAGTTCTGTCCAAGCCCGG | 684 |
| Hu.DMD.Exon51.25.015 | ATAACTTGATCAAGCAGAGAAAGCCA | 685 |
| Hu.DMD.Exon51.25.015.2 | AAGCAGAGAAAGCCAGTCGGTAAGT | 686 |
| Hu.DMD.Exon51.25.016 | CACCCTCTGTGATTTTATAACTTGAT | 687 |
| Hu.DMD.Exon51.25.017 | CAAGGTCACCCACCATCACCCTCTGT | 688 |
| Hu.DMD.Exon51.25.017.2 | CATCACCCTCTGTGATTTTATAACT | 689 |
| Hu.DMD.Exon51.25.018 | CTTCTGCTTGATGATCATCTCGTTGA | 690 |
| Hu.DMD.Exon51.25.019 | CCTTCTGCTTGATGATCATCTCGTTG | 691 |
| Hu.DMD.Exon51.25.019.2 | ATCTCGTTGATATCCTCAAGGTCACC | 692 |
| Hu.DMD.Exon51.25.020 | TCATACCTTCTGCTTGATGATCATCT | 693 |
| Hu.DMD.Exon51.25.020.2 | TCATTTTTTCTCATACCTTCTGCTTG | 694 |
| Hu.DMD.Exon51.25.021 | TTTTCTCATACCTTCTGCTTGATGAT | 695 |
| Hu.DMD.Exon51.25.022 | TTTTATCATTTTTTCTCATACCTTCT | 696 |
| Hu.DMD.Exon51.25.023 | CCAACTTTTATCATTTTTTCTCATAC | 697 |
| Hu.DMD.Exon51.20.001 | ATATTTTGGGTTTTTGCAAA | 698 |
| Hu.DMD.Exon51.20.002 | AAAATATTTTGGGTTTTTGC | 699 |
| Hu.DMD.Exon51.20.003 | GAGCTAAAATATTTTGGGTT | 700 |
| Hu.DMD.Exon51.20.004 | AGTAGGAGCTAAAATATTTT | 701 |
| Hu.DMD.Exon51.20.005 | GTCTGAGTAGGAGCTAAAAT | 702 |
| Hu.DMD.Exon51.20.006 | TAACAGTCTGAGTAGGAGCT | 703 |
| Hu.DMD.Exon51.20.007 | CAGAGTAACAGTCTGAGTAG | 704 |
| Hu.DMD.Exon51.20.008 | CACAGGTTGTGTCACCAGAG | 705 |
| Hu.DMD.Exon51.20.009 | AGTTTCCTTAGTAACCACAG | 706 |
| Hu.DMD.Exon51.20.010 | TAGTTTGGAGATGGCAGTTT | 707 |
| Hu.DMD.Exon51.20.011 | GGAAGATGGCATTTCTAGTT | 708 |
| Hu.DMD.Exon51.20.012 | TACCTCCAACATCAAGGAAG | 709 |
| Hu.DMD.Exon51.20.013 | ATCTGCCAGAGCAGGTACCT | 710 |
| Hu.DMD.Exon51.20.014 | CCAAGCCCGGTTGAAATCTG | 711 |
| Hu.DMD.Exon51.20.015 | GTCGGTAAGTTCTGTCCAAG | 712 |
| Hu.DMD.Exon51.20.016 | AAGCAGAGAAAGCCAGTCGG | 713 |
| Hu.DMD.Exon51.20.017 | TTTTATAACTTGATCAAGCA | 714 |
| Hu.DMD.Exon51.20.018 | CATCACCCTCTGTGATTTTA | 715 |
| Hu.DMD.Exon51.20.019 | CTCAAGGTCACCCACCATCA | 716 |
| Hu.DMD.Exon51.20.020 | CATCTCGTTGATATCCTCAA | 717 |
| Hu.DMD.Exon51.20.021 | CTTCTGCTTGATGATCATCT | 718 |
| Hu.DMD.Exon51.20.022 | CATACCTTCTGCTTGATGAT | 719 |
| Hu.DMD.Exon51.20.023 | TTTCTCATACCTTCTGCTTG | 720 |
| Hu.DMD.Exon51.20.024 | CATTTTTTCTCATACCTTCT | 721 |
| Hu.DMD.Exon51.20.025 | TTTATCATTTTTTCTCATAC | 722 |
| Hu.DMD.Exon51.20.026 | CAACTTTTATCATTTTTTCT | 723 |
| Hu.DMD.Exon52.25.001 | CTGTAAGAACAAATATCCCTTAGTA | 724 |
| Hu.DMD.Exon52.25.002 | TGCCTGTAAGAACAAATATCCCTTA | 725 |
| Hu.DMD.Exon52.25.002.2 | GTTGCCTGTAAGAACAAATATCCCT | 726 |
| Hu.DMD.Exon52.25.003 | ATTGTTGCCTGTAAGAACAAATATC | 727 |
| Hu.DMD.Exon52.25.003.2 | GCATTGTTGCCTGTAAGAACAAATA | 728 |
| Hu.DMD.Exon52.25.004 | CCTGCATTGTTGCCTGTAAGAACAA | 729 |
| Hu.DMD.Exon52.25.004.2 | ATCCTGCATTGTTGCCTGTAAGAAC | 730 |
| Hu.DMD.Exon52.25.005 | CAAATCCTGCATTGTTGCCTGTAAG | 731 |
| Hu.DMD.Exon52.25.005.2 | TCCAAATCCTGCATTGTTGCCTGTA | 732 |
| Hu.DMD.Exon52.25.006 | TGTTCCAAATCCTGCATTGTTGCCT | 733 |
| Hu.DMD.Exon52.25.006.2 | TCTGTTCCAAATCCTGCATTGTTGC | 734 |
| Hu.DMD.Exon52.25.007 | AACTGGGGACGCCTCTGTTCCAAAT | 735 |
| Hu.DMD.Exon52.25.007.2 | GCCTCTGTTCCAAATCCTGCATTGT | 736 |
| Hu.DMD.Exon52.25.008 | CAGCGGTAATGAGTTCTTCCAACTG | 737 |
| Hu.DMD.Exon52.25.008.2 | CTTCCAACTGGGGACGCCTCTGTTC | 738 |
| Hu.DMD.Exon52.25.009 | CTTGTTTTTCAAATTTTGGGCAGCG | 739 |
| Hu.DMD.Exon52.25.010 | CTAGCCTCTTGATTGCTGGTCTTGT | 740 |
| Hu.DMD.Exon52.25.010.2 | TTTTCAAATTTTGGGCAGCGGTAAT | 741 |
| Hu.DMD.Exon52.25.011 | TTCGATCCGTAATGATTGTTCTAGC | 742 |
| Hu.DMD.Exon52.25.011.2 | GATTGCTGGTCTTGTTTTTCAAATT | 743 |
| Hu.DMD.Exon52.25.012 | CTTACTTCGATCCGTAATGATTGTT | 744 |
| Hu.DMD.Exon52.25.012.2 | TTGTTCTAGCCTCTTGATTGCTGGT | 745 |
| Hu.DMD.Exon52.25.013 | AAAAACTTACTTCGATCCGTAATGA | 746 |
| Hu.DMD.Exon52.25.014 | TGTTAAAAAACTTACTTCGATCCGT | 747 |
| Hu.DMD.Exon52.25.015 | ATGCTTGTTAAAAAACTTACTTCGA | 748 |
| Hu.DMD.Exon52.25.016 | GTCCCATGCTTGTTAAAAAACTTAC | 749 |
| Hu.DMD.Exon52.20.001 | AGAACAAATATCCCTTAGTA | 750 |
| Hu.DMD.Exon52.20.002 | GTAAGAACAAATATCCCTTA | 751 |
| Hu.DMD.Exon52.20.003 | TGCCTGTAAGAACAAATATC | 752 |
| Hu.DMD.Exon52.20.004 | ATTGTTGCCTGTAAGAACAA | 753 |
| Hu.DMD.Exon52.20.005 | CCTGCATTGTTGCCTGTAAG | 754 |
| Hu.DMD.Exon52.20.006 | CAAATCCTGCATTGTTGCCT | 755 |
| Hu.DMD.Exon52.20.007 | GCCTCTGTTCCAAATCCTGC | 756 |
| Hu.DMD.Exon52.20.008 | CTTCCAACTGGGGACGCCTC | 757 |
| Hu.DMD.Exon52.20.009 | CAGCGGTAATGAGTTCTTCC | 758 |
| Hu.DMD.Exon52.20.010 | TTTTCAAATTTTGGGCAGCG | 759 |
| Hu.DMD.Exon52.20.011 | GATTGCTGGTCTTGTTTTTC | 760 |
| Hu.DMD.Exon52.20.012 | TTGTTCTAGCCTCTTGATTG | 761 |
| Hu.DMD.Exon52.20.013 | TTCGATCCGTAATGATTGTT | 762 |
| Hu.DMD.Exon52.20.014 | CTTACTTCGATCCGTAATGA | 763 |
| Hu.DMD.Exon52.20.015 | AAAAACTTACTTCGATCCGT | 764 |
| Hu.DMD.Exon52.20.016 | TGTTAAAAAACTTACTTCGA | 765 |
| Hu.DMD.Exon52.20.017 | ATGCTTGTTAAAAAACTTAC | 766 |
| Hu.DMD.Exon52.20.018 | GTCCCATGCTTGTTAAAAAA | 767 |
| Hu.DMD.Exon53.25.001 | CTAGAATAAAAGGAAAAATAAATAT | 768 |
| Hu.DMD.Exon53.25.002 | AACTAGAATAAAAGGAAAAATAAAT | 769 |
| Hu.DMD.Exon53.25.002.2 | TTCAACTAGAATAAAAGGAAAAATA | 770 |
| Hu.DMD.Exon53.25.003 | CTTTCAACTAGAATAAAAGGAAAAA | 771 |
| Hu.DMD.Exon53.25.003.2 | ATTCTTTCAACTAGAATAAAAGGAA | 772 |
| Hu.DMD.Exon53.25.004 | GAATTCTTTCAACTAGAATAAAAGG | 773 |
| Hu.DMD.Exon53.25.004.2 | TCTGAATTCTTTCAACTAGAATAAA | 774 |
| Hu.DMD.Exon53.25.005 | ATTCTGAATTCTTTCAACTAGAATA | 775 |
| Hu.DMD.Exon53.25.005.2 | CTGATTCTGAATTCTTTCAACTAGA | 776 |
| Hu.DMD.Exon53.25.006 | CACTGATTCTGAATTCTTTCAACTA | 777 |
| Hu.DMD.Exon53.25.006.2 | TCCCACTGATTCTGAATTCTTTCAA | 778 |
| Hu.DMD.Exon53.25.007 | CATCCCACTGATTCTGAATTCTTTC | 779 |
| Hu.DMD.Exon53.25.008 | TACTTCATCCCACTGATTCTGAATT | 780 |
| Hu.DMD.Exon53.25.008.2 | CTGAAGGTGTTCTTGTACTTCATCC | 781 |
| Hu.DMD.Exon53.25.009 | CGGTTCTGAAGGTGTTCTTGTACT | 782 |
| Hu.DMD.Exon53.25.009.2 | CTGTTGCCTCCGGTTCTGAAGGTGT | 783 |
| Hu.DMD.Exon53.25.010 | TTTCATTCAACTGTTGCCTCCGGTT | 784 |
| Hu.DMD.Exon53.25.010.2 | TAACATTTCATTCAACTGTTGCCTC | 785 |
| Hu.DMD.Exon53.25.011 | TTGTGTTGAATCCTTTAACATTTCA | 786 |
| Hu.DMD.Exon53.25.012 | TCTTCCTTAGCTTCCAGCCATTGTG | 787 |
| Hu.DMD.Exon53.25.012.2 | CTTAGCTTCCAGCCATTGTGTTGAA | 788 |
| Hu.DMD.Exon53.25.013 | GTCCTAAGACCTGCTCAGCTTCTTC | 789 |
| Hu.DMD.Exon53.25.013.2 | CTGCTCAGCTTCTTCCTTAGCTTCC | 790 |
| Hu.DMD.Exon53.25.014 | CTCAAGCTTGGCTCTGGCCTGTCCT | 791 |
| Hu.DMD.Exon53.25.014.2 | GGCCTGTCCTAAGACCTGCTCAGCT | 792 |
| Hu.DMD.Exon53.25.015 | TAGGGACCCTCCTTCCATGACTCAA | 793 |
| Hu.DMD.Exon53.25.016 | TTTGGATTGCATCTACTGTATAGGG | 794 |
| Hu.DMD.Exon53.25.016.2 | ACCCTCCTTCCATGACTCAAGCTTG | 795 |
| Hu.DMD.Exon53.25.017 | CTTGGTTTCTGTGATTTTCTTTTGG | 796 |
| Hu.DMD.Exon53.25.017.2 | ATCTACTGTATAGGGACCCTCCTTC | 797 |
| Hu.DMD.Exon53.25.018 | CTAACCTTGGTTTCTGTGATTTTCT | 798 |
| Hu.DMD.Exon53.25.018.2 | TTTCTTTTGGATTGCATCTACTGTA | 799 |
| Hu.DMD.Exon53.25.019 | TGATACTAACCTTGGTTTCTGTGAT | 800 |
| Hu.DMD.Exon53.25.020 | ATCTTTGATACTAACCTTGGTTTCT | 801 |
| Hu.DMD.Exon53.25.021 | AAGGTATCTTTGATACTAACCTTGG | 802 |
| Hu.DMD.Exon53.25.022 | TTAAAAAGGTATCTTTGATACTAAC | 803 |
| Hu.DMD.Exon53.20.001 | ATAAAAGGAAAAATAAATAT | 804 |
| Hu.DMD.Exon53.20.002 | GAATAAAAGGAAAAATAAAT | 805 |
| Hu.DMD.Exon53.20.003 | AACTAGAATAAAAGGAAAAA | 806 |
| Hu.DMD.Exon53.20.004 | CTTTCAACTAGAATAAAAGG | 807 |
| Hu.DMD.Exon53.20.005 | GAATTCTTTCAACTAGAATA | 808 |
| Hu.DMD.Exon53.20.006 | ATTCTGAATTCTTTCAACTA | 809 |
| Hu.DMD.Exon53.20.007 | TACTTCATCCCACTGATTCT | 810 |
| Hu.DMD.Exon53.20.008 | CTGAAGGTGTTCTTGTACT | 811 |
| Hu.DMD.Exon53.20.009 | CTGTTGCCTCCGGTTCTGAA | 812 |
| Hu.DMD.Exon53.20.010 | TAACATTTCATTCAACTGTT | 813 |
| Hu.DMD.Exon53.20.011 | TTGTGTTGAATCCTTTAACA | 814 |
| Hu.DMD.Exon53.20.012 | CTTAGCTTCCAGCCATTGTG | 815 |
| Hu.DMD.Exon53.20.013 | CTGCTCAGCTTCTTCCTTAG | 816 |
| Hu.DMD.Exon53.20.014 | GGCCTGTCCTAAGACCTGCT | 817 |
| Hu.DMD.Exon53.20.015 | CTCAAGCTTGGCTCTGGCCT | 818 |
| Hu.DMD.Exon53.20.016 | ACCCTCCTTCCATGACTCAA | 819 |
| Hu.DMD.Exon53.20.017 | ATCTACTGTATAGGGACCCT | 820 |
| Hu.DMD.Exon53.20.018 | TTTCTTTTGGATTGCATCTA | 821 |
| Hu.DMD.Exon53.20.019 | CTTGGTTTCTGTGATTTTCT | 822 |
| Hu.DMD.Exon53.20.020 | CTAACCTTGGTTTCTGTGAT | 823 |
| Hu.DMD.Exon53.20.021 | TGATACTAACCTTGGTTTCT | 824 |
| Hu.DMD.Exon53.20.022 | ATCTTTGATACTAACCTTGG | 825 |
| Hu.DMD.Exon53.20.023 | AAGGTATCTTTGATACTAAC | 826 |
| Hu.DMD.Exon53.20.024 | TTAAAAAGGTATCTTTGATA | 827 |
| Hu.DMD.Exon54.25.001 | CTATAGATTTTTATGAGAAAGAGA | 828 |
| Hu.DMD.Exon54.25.002 | AACTGCTATAGATTTTTATGAGAAA | 829 |
| Hu.DMD.Exon54.25.003 | TGGCCAACTGCTATAGATTTTTATG | 830 |
| Hu.DMD.Exon54.25.004 | GTCTTTGGCCAACTGCTATAGATTT | 831 |
| Hu.DMD.Exon54.25.005 | CGGAGGTCTTTGGCCAACTGCTATA | 832 |
| Hu.DMD.Exon54.25.006 | ACTGGCGGAGGTCTTTGGCCAACTG | 833 |
| Hu.DMD.Exon54.25.007 | TTTGTCTGCCACTGGCGGAGGTCTT | 834 |
| Hu.DMD.Exon54.25.008 | AGTCATTTGCCACATCTACATTTGT | 835 |
| Hu.DMD.Exon54.25.008.2 | TTTGCCACATCTACATTTGTCTGCC | 836 |
| Hu.DMD.Exon54.25.009 | CCGGAGAAGTTTCAGGGCCAAGTCA | 837 |
| Hu.DMD.Exon54.25.010 | GTATCATCTGCAGAATAATCCCGGA | 838 |
| Hu.DMD.Exon54.25.010.2 | TAATCCCGGAGAAGTTTCAGGGCCA | 839 |
| Hu.DMD.Exon54.25.011 | TTATCATGTGGACTTTTCTGGTATC | 840 |
| Hu.DMD.Exon54.25.012 | AGAGGCATTGATATTCTCTGTTATC | 841 |
| Hu.DMD.Exon54.25.012.2 | ATGTGGACTTTTCTGGTATCATCTG | 842 |
| Hu.DMD.Exon54.25.013 | CTTTTATGAATGCTTCTCCAAGAGG | 843 |
| Hu.DMD.Exon54.25.013.2 | ATATTCTCTGTTATCATGTGGACTT | 844 |
| Hu.DMD.Exon54.25.014 | CATACCTTTTATGAATGCTTCTCCA | 845 |
| Hu.DMD.Exon54.25.014.2 | CTCCAAGAGGCATTGATATTCTCTG | 846 |
| Hu.DMD.Exon54.25.015 | TAATTCATACCTTTTATGAATGCTT | 847 |
| Hu.DMD.Exon54.25.015.2 | CTTTTATGAATGCTTCTCCAAGAGG | 848 |
| Hu.DMD.Exon54.25.016 | TAATGTAATTCATACCTTTTATGAA | 849 |
| Hu.DMD.Exon54.25.017 | AGAAATAATGTAATTCATACCTTTT | 850 |
| Hu.DMD.Exon54.25.018 | GTTTTAGAAATAATGTAATTCATAC | 851 |
| Hu.DMD.Exon54.20.001 | GATTTTTATGAGAAAGAGA | 852 |
| Hu.DMD.Exon54.20.002 | CTATAGATTTTTATGAGAAA | 853 |
| Hu.DMD.Exon54.20.003 | AACTGCTATAGATTTTTATG | 854 |
| Hu.DMD.Exon54.20.004 | TGGCCAACTGCTATAGATTT | 855 |
| Hu.DMD.Exon54.20.005 | GTCTTTGGCCAACTGCTATA | 856 |
| Hu.DMD.Exon54.20.006 | CGGAGGTCTTTGGCCAACTG | 857 |
| Hu.DMD.Exon54.20.007 | TTTGTCTGCCACTGGCGGAG | 858 |
| Hu.DMD.Exon54.20.008 | TTTGCCACATCTACATTTGT | 859 |
| Hu.DMD.Exon54.20.009 | TTCAGGGCCAAGTCATTTGC | 860 |
| Hu.DMD.Exon54.20.010 | TAATCCCGGAGAAGTTTCAG | 861 |
| Hu.DMD.Exon54.20.011 | GTATCATCTGCAGAATAATC | 862 |
| Hu.DMD.Exon54.20.012 | ATGTGGACTTTTCTGGTATC | 863 |
| Hu.DMD.Exon54.20.013 | ATATTCTCTGTTATCATGTG | 864 |
| Hu.DMD.Exon54.20.014 | CTCCAAGAGGCATTGATATT | 865 |
| Hu.DMD.Exon54.20.015 | CTTTTATGAATGCTTCTCCA | 866 |
| Hu.DMD.Exon54.20.016 | CATACCTTTTATGAATGCTT | 867 |
| Hu.DMD.Exon54.20.017 | TAATTCATACCTTTTATGAA | 868 |
| Hu.DMD.Exon54.20.018 | TAATGTAATTCATACCTTTT | 869 |
| Hu.DMD.Exon54.20.019 | AGAAATAATGTAATTCATAC | 870 |
| Hu.DMD.Exon54.20.020 | GTTTTAGAAATAATGTAATT | 871 |
| Hu.DMD.Exon55.25.001 | CTGCAAAGGACCAAATGTTCAGATG | 872 |
| Hu.DMD.Exon55.25.002 | TCACCCTGCAAAGGACCAAATGTTC | 873 |
| Hu.DMD.Exon55.25.003 | CTCACTCACCCTGCAAAGGACCAAA | 874 |
| Hu.DMD.Exon55.25.004 | TCTCGCTCACTCACCCTGCAAAGGA | 875 |
| Hu.DMD.Exon55.25.005 | CAGCCTCTCGCTCACTCACCCTGCA | 876 |
| Hu.DMD.Exon55.25.006 | CAAAGCAGCCTCTCGCTCACTCACC | 877 |
| Hu.DMD.Exon55.25.007 | TCTTCCAAAGCAGCCTCTCGCTCAC | 878 |
| Hu.DMD.Exon55.25.007.2 | TCTATGAGTTTCTTCCAAAGCAGCC | 879 |
| Hu.DMD.Exon55.25.008 | GTTGCAGTAATCTATGAGTTTCTTC | 880 |
| Hu.DMD.Exon55.25.008.2 | GAACTGTTGCAGTAATCTATGAGTT | 881 |
| Hu.DMD.Exon55.25.009 | TTCCAGGTCCAGGGGGAACTGTTGC | 882 |
| Hu.DMD.Exon55.25.010 | GTAAGCCAGGCAAGAAACTTTTCCA | 883 |
| Hu.DMD.Exon55.25.010.2 | CCAGGCAAGAAACTTTTCCAGGTCC | 884 |
| Hu.DMD.Exon55.25.011 | TGGCAGTTGTTTCAGCTTCTGTAAG | 885 |
| Hu.DMD.Exon55.25.011.2 | TTCAGCTTCTGTAAGCCAGGCAAGA | 886 |
| Hu.DMD.Exon55.25.012 | GGTAGCATCCTGTAGGACATTGGCA | 887 |
| Hu.DMD.Exon55.25.012.2 | GACATTGGCAGTTGTTTCAGCTTCT | 888 |
| Hu.DMD.Exon55.25.013 | TCTAGGAGCCTTTCCTTACGGGTAG | 889 |
| Hu.DMD.Exon55.25.014 | CTTTTACTCCCTTGGAGTCTTCTAG | 890 |
| Hu.DMD.Exon55.25.014.2 | GAGCCTTTCCTTACGGGTAGCATCC | 891 |
| Hu.DMD.Exon55.25.015 | TTGCCATTGTTTCATCAGCTCTTTT | 892 |
| Hu.DMD.Exon55.25.015.2 | CTTGGAGTCTTCTAGGAGCCTTTCC | 893 |
| Hu.DMD.Exon55.25.016 | CTTACTTGCCATTGTTTCATCAGCT | 894 |
| Hu.DMD.Exon55.25.016.2 | CAGCTCTTTTACTCCCTTGGAGTCT | 895 |
| Hu.DMD.Exon55.25.017 | CCTGACTTACTTGCCATTGTTTCAT | 896 |
| Hu.DMD.Exon55.25.018 | AAATGCCTGACTTACTTGCCATTGT | 897 |
| Hu.DMD.Exon55.25.019 | AGCGGAAATGCCTGACTTACTTGCC | 898 |
| Hu.DMD.Exon55.25.020 | GCTAAAGCGGAAATGCCTGACTTAC | 899 |
| Hu.DMD.Exon55.20.001 | AAGGACCAAATGTTCAGATG | 900 |
| Hu.DMD.Exon55.20.002 | CTGCAAAGGACCAAATGTTC | 901 |
| Hu.DMD.Exon55.20.003 | TCACCCTGCAAAGGACCAAA | 902 |
| Hu.DMD.Exon55.20.004 | CTCACTCACCCTGCAAAGGA | 903 |
| Hu.DMD.Exon55.20.005 | TCTCGCTCACTCACCCTGCA | 904 |
| Hu.DMD.Exon55.20.006 | CAGCCTCTCGCTCACTCACC | 905 |
| Hu.DMD.Exon55.20.007 | CAAAGCAGCCTCTCGCTCAC | 906 |
| Hu.DMD.Exon55.20.008 | TCTATGAGTTTCTTCCAAAG | 907 |
| Hu.DMD.Exon55.20.009 | GAACTGTTGCAGTAATCTAT | 908 |
| Hu.DMD.Exon55.20.010 | TTCCAGGTCCAGGGGGAACT | 909 |
| Hu.DMD.Exon55.20.011 | CCAGGCAAGAAACTTTTCCA | 910 |
| Hu.DMD.Exon55.20.012 | TTCAGCTTCTGTAAGCCAGG | 911 |
| Hu.DMD.Exon55.20.013 | GACATTGGCAGTTGTTTCAG | 912 |
| Hu.DMD.Exon55.20.014 | GGTAGCATCCTGTAGGACAT | 913 |
| Hu.DMD.Exon55.20.015 | GAGCCTTTCCTTACGGGTAG | 914 |
| Hu.DMD.Exon55.20.016 | CTTGGAGTCTTCTAGGAGCC | 915 |
| Hu.DMD.Exon55.20.017 | CAGCTCTTTTACTCCCTTGG | 916 |
| Hu.DMD.Exon55.20.018 | TTGCCATTGTTTCATCAGCT | 917 |
| Hu.DMD.Exon55.20.019 | CTTACTTGCCATTGTTTCAT | 918 |
| Hu.DMD.Exon55.20.020 | CCTGACTTACTTGCCATTGT | 919 |
| Hu.DMD.Exon55.20.021 | AAATGCCTGACTTACTTGCC | 920 |
| Hu.DMD.Exon55.20.022 | AGCGGAAATGCCTGACTTAC | 921 |
| Hu.DMD.Exon55.20.023 | GCTAAAGCGGAAATGCCTGA | 922 |
| H50A(+02+30)-AVI-5656 | CCACTCAGAGCTCAGATCTTCTAACTTCC | 923 |
| H50D(+07−18)-AVI-5915 | GGGATCCAGTATACTTACAGGCTCC | 924 |
| H50A(+07+33) | CTTCCACTCAGAGCTCAGATCTTCTAA | 925 |
| H51A(+61+90)-AVI-4657 | ACATCAAGGAAGATGGCATTTCTAGTTTGG | 926 |
| H51A(+66+95)-AVI-4658 | CTCCAACATCAAGGAAGATGGCATTTCTAG | 927 |
| H51A(+111+134) | TTCTGTCCAAGCCCGGTTGAAATC | 928 |
| H51A(+175+195) | CACCCACCATCACCCTCYGTG | 929 |
| H51A(+199+220) | ATCATCTCGTTGATATCCTCAA | 930 |
| H51A(+66+90) | ACATCAAGGAAGATGGCATTTCTAG | 931 |
| H51A(−01+25) | ACCAGAGTAACAGTCTGAGTAGGAGC | 932 |
| h51AON1 | TCAAGGAAGATGGCATTTCT | 933 |
| h51AON2 | CCTCTGTGATTTTATAACTTGAT | 934 |
| H51D(+08−17) | ATCATTTTTTCTCATACCTTCTGCT | 935 |
| H51D(+16−07) | CTCATACCTTCTGCTTGATGATC | 936 |
| hAON#23 | TGGCATTTCTAGTTTGG | 937 |
| hAON#24 | CCAGAGCAGGTACCTCCAACATC | 938 |
| H44A(+61+84) | TGTTCAGCTTCTGTTAGCCACTGA | 939 |
| H44A(+85+104) | TTTGTGTCTTTCTGAGAAAC | 940 |
| h44AON1 | CGCCGCCATTTCTCAACAG | 941 |
| H44A(−06+14) | ATCTGTCAAATCGCCTGCAG | 942 |
| H45A(+71+90) | TGTTTTTGAGGATTGCTGAA | 943 |
| h45AON1 | GCTGAATTATTTCTTCCCC | 944 |
| h45AON5 | GCCCAATGCCATCCTGG | 945 |
| H45A(−06+20) | CCAATGCCATCCTGGAGTTCCTGTAA | 946 |
| H53A(+39+69) | CATTCAACTGTTGCCTCCGGTTCTGAAGGTG | 947 |
| H53A(+23+47) | CTGAAGGTGTTCTTGTACTTCATCC | 948 |
| h53AON1 | CTGTTGCCTCCGGTTCTG | 949 |
| H53A(−12+10) | ATTCTTTCAACTAGAATAAAAG | 950 |
| huEx45.30.66 | GCCATCCTGGAGTTCCTGTAAGATACCAAA | 951 |
| huEx45.30.71 | CCAATGCCATCCTGGAGTTCCTGTAAGATA | 952 |
| huEx45.30.79 | GCCGCTGCCCAATGCCATCCTGGAGTTCCT | 953 |
| huEx45.30.83 | GTTTGCCGCTGCCCAATGCCATCCTGGAGT | 954 |
| huEx45.30.88 | CAACAGTTTGCCGCTGCCCAATGCCATCCT | 955 |
| huEx45.30.92 | CTGACAACAGTTTGCCGCTGCCCAATGCCA | 956 |
| huEx45.30.96 | TGTTCTGACAACAGTTTGCCGCTGCCCAAT | 957 |
| huEx45.30.99 | CAATGTTCTGACAACAGTTTGCCGCTGCCC | 958 |
| huEx45.30.103 | CATTCAATGTTCTGACAACAGTTTGCCGCT | 959 |
| huEx45.30.120 | TATTTCTTCCCCAGTTGCATTCAATGTTCT | 960 |
| huEx45.30.127 | GCTGAATTATTTCTTCCCCAGTTGCATTCA | 961 |
| huEx45.30.132 | GGATTGCTGAATTATTTCTTCCCCAGTTGC | 962 |
| huEx45.30.137 | TTTGAGGATTGCTGAATTATTTCTTCCCCA | 963 |
| huEx53.30.84 | GTACTTCATCCCACTGATTCTGAATTCTTT | 964 |
| huEx53.30.88 | TCTTGTACTTCATCCCACTGATTCTGAATT | 965 |
| huEx53.30.91 | TGTTCTTGTACTTCATCCCACTGATTCTGA | 966 |
| huEx53.30.103 | CGGTTCTGAAGGTGTTCTTGTACTTCATCC | 967 |
| huEx53.30.106 | CTCCGGTTCTGAAGGTGTTCTTGTACTTCA | 968 |
| huEx53.30.109 | TGCCTCCGGTTCTGAAGGTGTTCTTGTACT | 969 |
| huEx53.30.112 | TGTTGCCTCCGGTTCTGAAGGTGTTCTTGT | 970 |
| huEx53.30.115 | AACTGTTGCCTCCGGTTCTGAAGGTGTTCT | 971 |
| huEx53.30.118 | TTCAACTGTTGCCTCCGGTTCTGAAGGTGT | 972 |

### Step 1: Antibody Conjugation with Maleimide-PEG-NHS Followed by siRNA-DMD Conjugates

Anti-dystrophin antibody is exchanged with 1× Phosphate buffer (pH 7.4) and made up to 5 mg/ml concentration. To this solution, 2 equivalents of SMCC linker or maleimide-PEGxkDa-NHS (x=1, 5, 10, 20) is added and rotated for 4 hours at room temperature. Unreacted maleimide-PEG is removed by spin filtration using 50 kDa MWCO Amicon spin filters and PBS pH 7.4. The antibody-PEG-Mal conjugate is collected and transferred into a reaction vessel.

Various siRNA conjugates are synthesized using sequences listed in Tables 13-17. siRNA-DMD conjugates (2 equivalents) is added at RT to the antibody-PEG-maleimide in PBS and rotated overnight. The reaction mixture is analyzed by analytical SAX column chromatography and conjugate along with unreacted antibody and siRNA is seen.

### Step 2: Purification

The crude reaction mixture is purified by AKTA explorer FPLC using anion exchange chromatography. Fractions containing the antibody-PEG-DMD conjugate are pooled, concentrated and buffer exchanged with PBS, pH 7.4. Antibody siRNA conjugates with SMCC linker, PEG1 kDa, PEG5 kDa and PEG10 kDa are separated based on the siRNA loading.

### Step-3: Analysis of the Purified Conjugate

The isolated conjugate is characterized by either mass spec or SDS-PAGE. The purity of the conjugate is assessed by analytical HPLC using anion exchange chromatography.

### Example 13. Synthesis, Purification, and Characterization of Membrane Penetrating Peptide-Modified Antisense Phosphorodiamidate Morpholino Oligomers (PPMOs)

### Step 1. Synthesis of Ac-(RXR)4XB-mEx23PMO-N3

Lyophilized 5′-NH2 and 3′-azide functionalized PMO for skipping mouse exon 23 (mEx23 PMO, 18 mg, sequence: GGCCAAACCTCGGCTTACCTGAAAT (SEQ ID NO: 28)) was dissolved in anhydrous dimethyl sulfoxide (DMSO, 240 uL). In a separate vial, Ac-(RXR)4XB peptide (“(RXR)4XB” disclosed as SEQ ID NO: 1065) (4 molar equivalents, 15.8 mg) and 1-[Bis(dimethylamino)methylene]-1H-1,2,3-triazolo[4,5-b]pyridinium 3-oxide hexafluorophosphate (HATU, 4 molar equivalents, 3.1 mg) were dissolved in a mixture of anhydrous DMSO (240 uL) and N,N-Diisopropylethylamine (DIPEA, 10 molar equivalents, 17.7 uL) and let sit for 5 minutes. The PMO solution and the peptide solution were combined and the reaction was left for 2 hours at room temperature. The reaction mixture was diluted to 15 mL with nanopure water. The reaction was concentrated in a 15 mL 3,000 MWCO Amicon spin filter (4,000×g for 30 min), diluted to 15 mL with nanopure water, and repeated for a total of 3 washes. The final retentate was not diluted with water. The reaction mixture was purified by SCX purification following SCX method 2. The pooled fractions containing product were concentrated in a 15 mL 3,000 MWCO Amicon spin filter (4,000×g for 30 min), diluted to 15 mL with nanopure water, and repeated for a total of 5 washes. The final retentate was not diluted with water. The product was then filtered through a sterile 0.22 um syringe filter and lyophilized in a pre-weighed vial. The product was recovered as a white powder (9.9 mg, 45% yield). The product was characterized by RP-HPLC following RP-HPLC method 1.

### Step 2. Purification of Ac-(RXR)4XB-mEx23PMO-N3

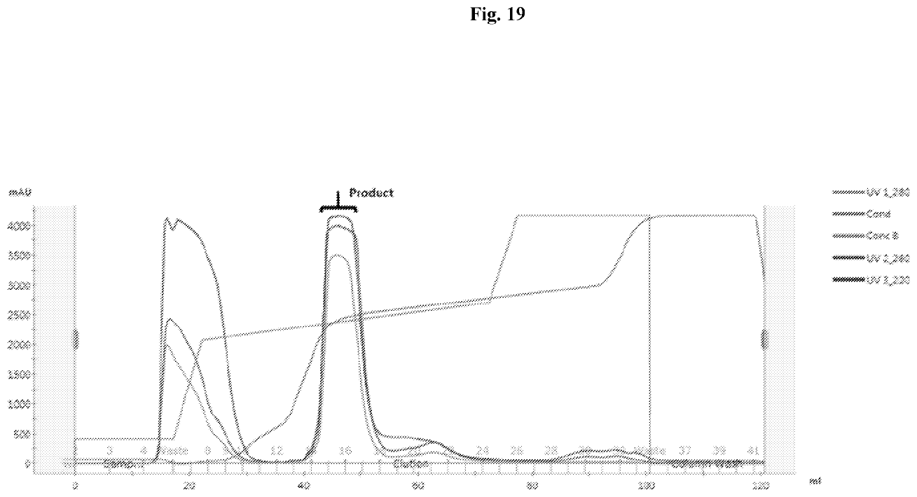

The reaction mixture was purified with an AKTA Explorer FPLC using SCX method 1. Fractions containing the desired product were combined and concentrated with Amicon Ultra-15 centrifugal filter units with a MWCO of 3500 kDa and buffer exchanged with PBS (pH 7.4) using Amicon Ultra-15 centrifugal filter units prior to analysis. FIG. 19 shows the SCX chromatogram of the PPMO product using SCX method 1.

### Step 3: Analysis of Purified PPMO

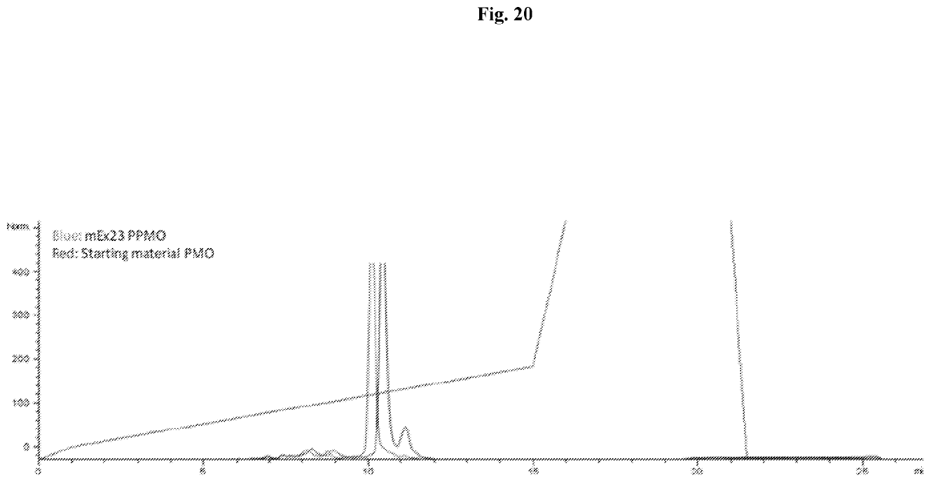

Analysis of the PPMO was performed with RP-HPLC using RP-HPLC method 1. FIG. 20 shows the RP-PHLC chromatogram of PMO starting material (1) and PPMO product (2) using HPLC method 1.

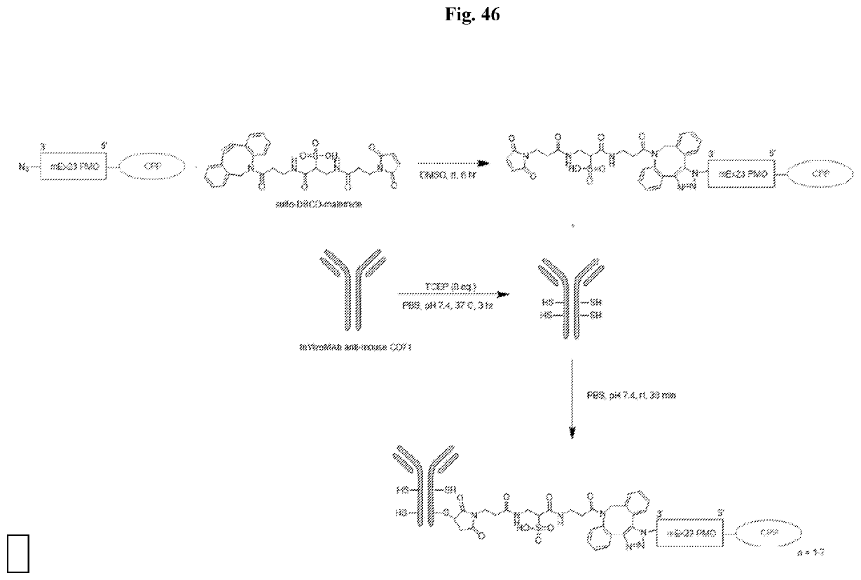

FIG. 46 illustrates an example of the synthetic strategy to produce PPMO-antibody oligonucleotide conjugates using a sulfo-DBCO-maleimide linker.

### Example 14. Synthesis, Purification, and Characterization of Membrane Penetrating Peptide-Modified Antisense Phosphorodiamidate Morpholino Oligomers Conjugated to a CD71 Antibody (PPMO-AOC) to Produce a Low Average Drug to Antibody Ratio (DAR) Using a Sulfo-DBCO-Maleimide Linker

### Anti-Transferrin Receptor Antibody

Anti-mouse transferrin receptor antibody or anti-CD71 mAb that was used was a rat IgG2a subclass monoclonal antibody that binds mouse CD71 or mouse transferrin receptor 1 (mTfR1). The antibody was produced by BioXcell and it is commercially available (Catalog #BE0175).

### Step 1: CD71 mAb-PPMO Conjugation

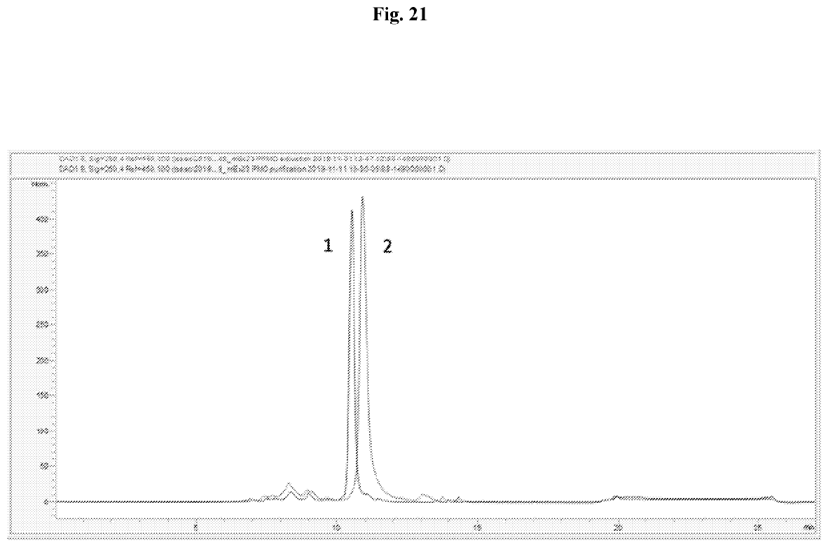

PPMO containing a 3′-azide (mEx23 PPMO, sequence: GGCCAAACCTCGGCTTACCTGAAAT (SEQ ID NO:28)) as a lyophilized solid was reacted with sulfo DBCO-Maleimide (4 equivalents) in anhydrous DMSO (250 um) for 6 hours. Excess sulfo DBCO-maleimide was removed by ultrafiltration using Amicon Ultra-15 centrifugal filter units with a MWCO of 3 kDa. The PPMO-DBCO-maleimide was washed 3 times with acetate buffer (10 mM sodium acetate, pH 6.0) and used immediately. CD71 Antibody (10 mg/ml) in PBS buffer (137 mM NaCl, 2.7 mM KCl, 10 mM Na2HPO4, and 1.8 mM KH2PO4), pH 7.4) was reduced by adding 8 equivalents of tris(2-carboxyethyl)phosphine (TCEP) in water and incubated at 37° C. for 2 hours. The reduced antibody was mixed with 1 equivalent of PPMO-DBCO-maleimide and incubated for 30 minutes at 25° C. N-Ethylmaleimide (8 equivalents) of was added to the mixture at room temperature for 30 minutes to quench unreacted cysteines. FIG. 21 shows RP-HPLC of PPMO-DBCO-maleimide reaction showing the PPMO starting material (1) and the PPMO-sulfoDBCO-maleimide (2). Data was acquired using reversed-phase HPLC method 1.

### Step 2: CD71 mAb-PPMO Purification

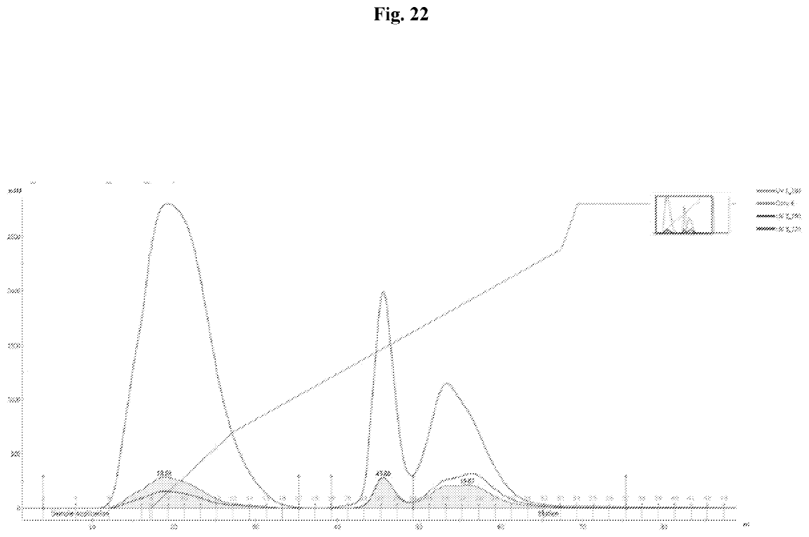

The reaction mixture was purified with an AKTA Explorer FPLC using SCX method 3. Fractions containing PPMO conjugated to the antibody were combined and concentrated with Amicon Ultra-15 centrifugal filter units with a MWCO of 50 kDa. The conjugate was concentrated and buffer exchanged with PBS (pH 7.4) using Amicon Ultra-15 centrifugal filter units prior to analysis. FIG. 22 shows SCX chromatogram of the CD71 mAb-PPMO purification using SCX method 3.

### Step 3: Analysis of Purified CD71 mAb-PPMO

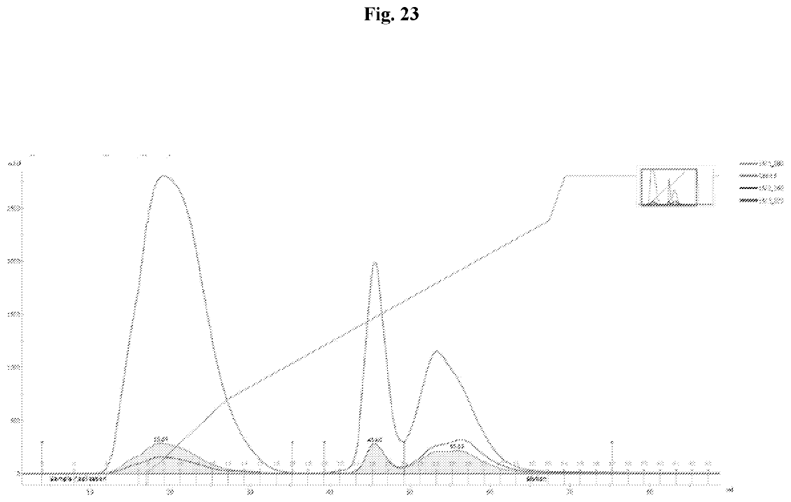

Analysis of the CD71 mAb-PPMO was performed with UV-Vis and SCX. The 260/280 nm UV absorbance ratio of each sample was compared to a standard sample of unreacted Ab. SCX method 2 was used to assess that the average DAR was 1.7 with 1.5% unreacted antibody remaining in the mixture. FIG. 23 shows an analysis of purified DAR 1.7 CD71 mAb-PPMO using SCX method 3.

### Example 15. Synthesis, Purification, and Characterization of Membrane Penetrating Peptide-Modified Antisense Phosphorodiamidate Morpholino Oligomers Conjugated to a CD71 Antibody (PPMO-AOC) to Produce a High Average Drug to Antibody Ratio (DAR) Using a Sulfo-DBCO-Maleimide Linker

### Step 1: CD71 mAb-PPMO Conjugation

PPMO containing a 3′ azide as a lyophilized solid was reacted with sulfo DBCO-Maleimide (4 equivalents) in anhydrous DMSO (250 um) for 6 hours. Excess sulfo DBCO-maleimide was removed by ultrafiltration using Amicon Ultra-15 centrifugal filter units with a MWCO of 3 kDa. The PPMO-DBCO-maleimide was washed three times with acetate buffer (10 mM sodium acetate, pH 6.0) and used immediately. CD71 Antibody (10 mg/ml) in PBS buffer (137 mM NaCl, 2.7 mM KCl, 10 mM Na2HPO4, and 1.8 mM KH2PO4), pH 7.4) was reduced by adding 8 equivalents of tris(2-carboxyethyl)phosphine (TCEP) in water and incubating at 37° C. for 2 hours. The reduced antibody was mixed with 4 equivalents of PPMO-DBCO-maleimide and incubated for 30 minutes at 25° C. N-Ethylmaleimide (8 equivalents) of was added to the mixture at room temperature for 30 minutes to quench unreacted cysteines.

### Step 2: CD71 mAb-PPMO Purification

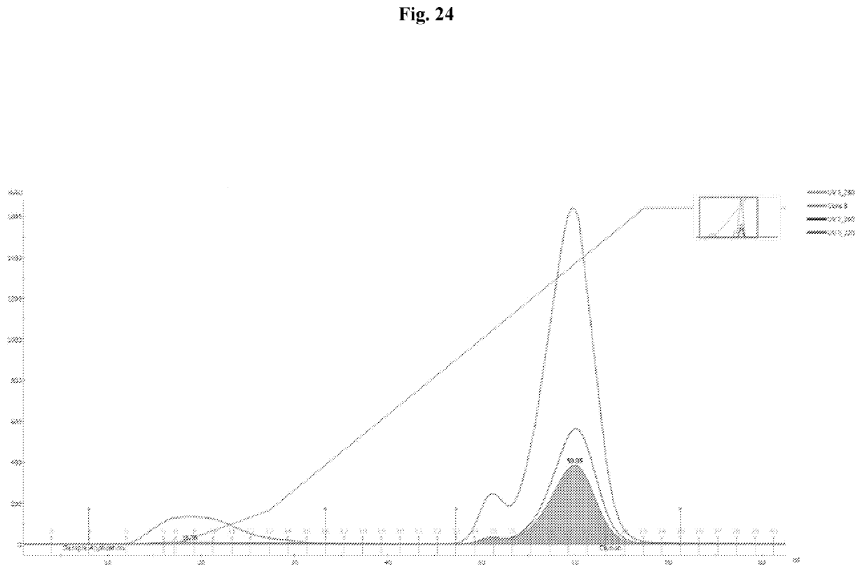

The reaction mixture was purified with an AKTA Explorer FPLC using SCX method 3. Fractions containing PPMO conjugated to the antibody were combined and concentrated with Amicon Ultra-15 centrifugal filter units with a MWCO of 50 kDa. The conjugate was concentrated and buffer exchanged with PBS (pH 7.4) using Amicon Ultra-15 centrifugal filter units prior to analysis. FIG. 24 shows a SCX chromatogram of the CD71 mAb-PPMO purification using SCX method 3.

### Step 3: Analysis of Purified CD71 mAb-PPMO

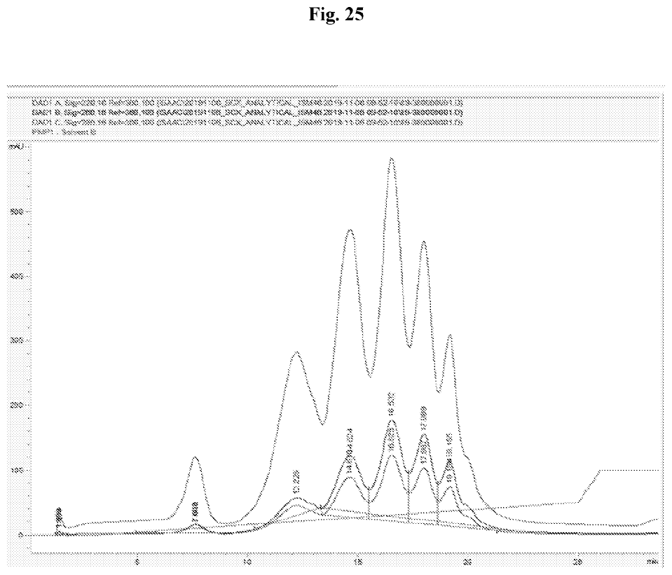

Analysis of the CD71 mAb-PPMO was performed with UV-Vis and SCX. The 260/280 nm UV absorbance ratio of each sample was compared to a standard sample of unreacted Ab. SCX method 2 was used to assess that the average DAR was 3.5 with 0.3% unreacted antibody remaining in the mixture. FIG. 25 show an analysis of purified DAR 3.5 CD71 mAb-PPMO using SCX method 2.

### Example 16. Production and Characterization of the CD71 Antibody Peptide Morpholino Antisense Oligonucleotide Conjugate (CD71 mAb-PPMO) Using a DBCO-PEG4-TFP Ester Linker

### Step 1: PPMO Synthesis

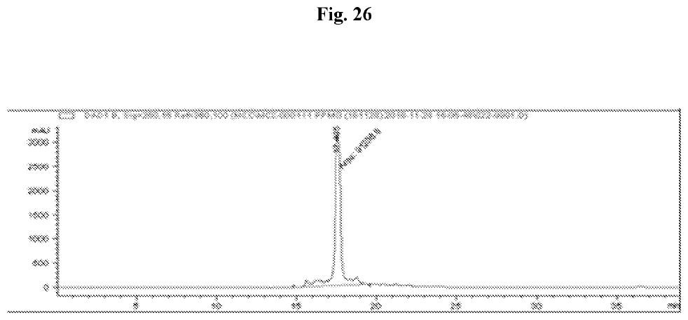

PMO with a 5′ primary amine and 3′ azide was dissolved in DMSO at 75 mg/ml was mixed with 2 equivalents of peptide (Ac—RXRRXRRXRRXRXB—COOH (“RXRRXRRXRRXRXB” disclosed as SEQ ID NO: 1066)) dissolved at 75 mg/ml in MDSO and 2 EQ HATU dissolved at 100 mg/ml in DMSO and 5 EQ DIPEA. Unreacted PMO was removed by SCX using SCX method 2. Purified PPMO was analyzed by SCX method 4. FIG. 26 shows the Chromatogram of PPMO produced using SCX method 4.

### Step 2: CD71 mAb-PPMO Conjugation

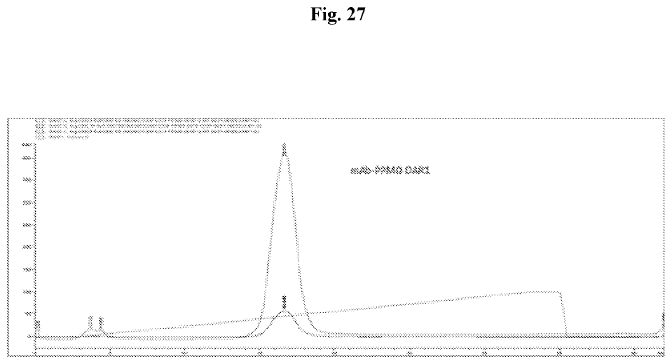

Antibody (7.26 mg/ml) in phosphate buffer pH 7.2 was mixed with 5 equivalents of DBCO-PEG4-TFP ester and incubated at room temperature for 1 hour. Excess linker was removed by ultrafiltration using Amicon Ultra-15 centrifugal filter units with a MWCO of 50 kDa. The antibody was washed three times with PBS before adding 3.5 equivalents of PPMO dropwise and incubated at 4° C. overnight. The reaction mixture was purified with an AKTA Explorer FPLC using SCX method 5. Fractions containing conjugates with a drug to antibody of one (DAR 1) and DAR≥2 were collected separately and concentrated with Amicon Ultra-15 centrifugal filter units with a MWCO of 50 kDa. Concentrated conjugates were buffer exchanged with PBS (pH 7.4) using Amicon Ultra-15 centrifugal filter units. FIG. 27 shows the chromatogram of mAb-PPMO DAR1 produced using SCX method 4.

### Example 17. Production and Characterization of the CD71 Antibody Morpholino Antisense Oligonucleotide Conjugates (CD71 mAb-PMO)

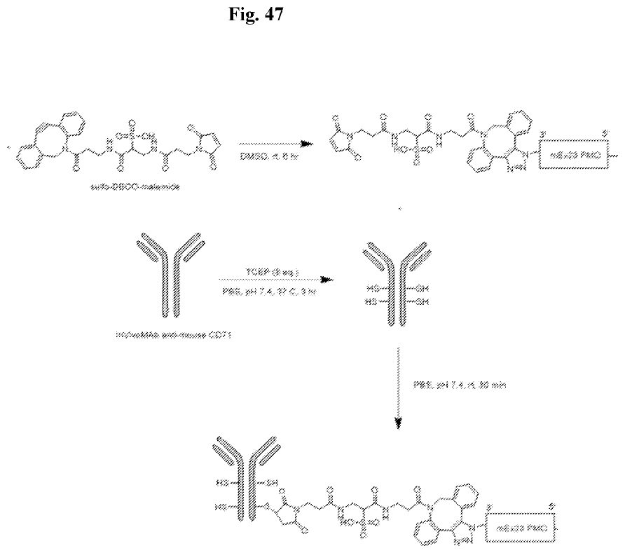

FIG. 47 illustrates an example of the synthetic strategy to produce PMO-antibody oligonucleotide conjugates (PMO-AOCs) using a sulfo-DBCO-maleimide linker.

### Example 18. Synthesis, Purification, and Characterization of Antisense Phosphorodiamidate Morpholino Oligomers Conjugated to a CD71 Antibody (PMO-AOC) to Produce a Low Average Drug to Antibody Ratio (DAR) Using a Sulfo-DBCO-Maleimide Linker

### Step 1: CD71 mAb-PMO Conjugation

PMO containing a 3′ azide as a lyophilized solid was reacted with sulfo DBCO-Maleimide (4 equivalents) in anhydrous DMSO (250 um) for 6 hours. Excess sulfo DBCO-maleimide was removed by ultrafiltration using Amicon Ultra-15 centrifugal filter units with a MWCO of 3 kDa. The PMO-DBCO-maleimide was washed three times with acetate buffer (10 mM sodium acetate, pH 6.0) and used immediately. CD71 Antibody (10 mg/ml) in PBS buffer (137 mM NaCl, 2.7 mM KCl, 10 mM Na2HPO4, and 1.8 mM KH2PO4), pH 7.4) was reduced by adding 8 equivalents of tris(2-carboxyethyl)phosphine (TCEP) in water and incubating at 37° C. for 2 hours. The reduced antibody was mixed with 1 equivalents of PMO-DBCO-maleimide and incubated for 30 minutes at 25° C. N-Ethylmaleimide (8 equivalents) of was added to the mixture at room temperature for 30 minutes to quench unreacted cysteines.

### Step 2: CD71 mAb-PMO Purification

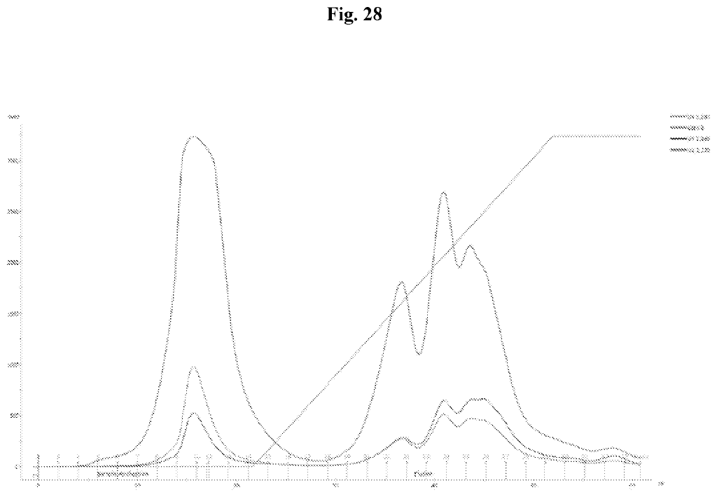

The reaction mixture was purified with an Agilent 1200 using HIC method 2. Fractions containing PMO conjugated to the antibody were combined and concentrated with Amicon Ultra-15 centrifugal filter units with a MWCO of 50 kDa The conjugate was concentrated and buffer exchanged with PBS (pH 7.4) using Amicon Ultra-15 centrifugal filter units prior to analysis. FIG. 28 shows the HIC chromatogram of the CD71 mAb-PMO purification using HIC method 2.

### Step 3: Analysis of Purified CD71 mAb-PMO

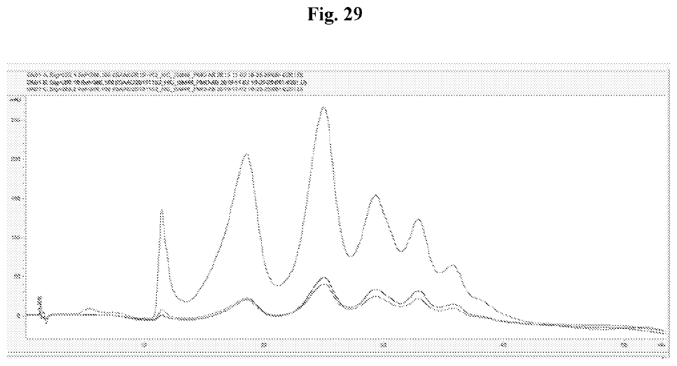

Analysis of the CD71 mAb-PMO was performed with UV-Vis, SCX and SEC. The 260/280 nm UV absorbance ratio of each sample was compared to a standard sample of unreacted Ab. HIC method 1 was used to assess that the average DAR was 2.5 with 5.4% unreacted antibody remaining in the mixture. SEC method 1 was to confirm the absence of high molecular weight aggregates and unconjugated PMO. FIG. 29 shows the analysis of purified low DAR CD71 mAb-PMO using HIC method 1.

### Example 19. Synthesis, Purification, and Characterization of Antisense Phosphorodiamidate Morpholino Oligomers Conjugated to a CD71 Antibody (PPMO-AOC) to Produce a High Average Drug to Antibody Ratio (DAR) Using a Sulfo-DBCO-Maleimide Linker

### Step 1: CD71 mAb-PMO Conjugation

PMO containing a 3′ azide as a lyophilized solid was reacted with sulfo DBCO-Maleimide (4 equivalents) in anhydrous DMSO (250 um) for 6 hours. Excess sulfo DBCO-maleimide was removed by ultrafiltration using Amicon Ultra-15 centrifugal filter units with a MWCO of 3 kDa. The PMO-DBCO-maleimide was washed three times with acetate buffer (10 mM sodium acetate, pH 6.0) and used immediately. CD71 Antibody (10 mg/ml) in PBS buffer (137 mM NaCl, 2.7 mM KCl, 10 mM Na2HPO4, and 1.8 mM KH2PO4), pH 7.4) was reduced by adding 8 equivalents of tris(2-carboxyethyl)phosphine (TCEP) in water and incubating at 37° C. for 2 hours. The reduced antibody was mixed with 4 equivalents of PMO-DBCO-maleimide and incubated for 30 minutes at 25° C. N-Ethylmaleimide (8 equivalents) of was added to the mixture at room temperature for 30 minutes to quench unreacted cysteines.

### Step 2: CD71 mAb-PMO Purification

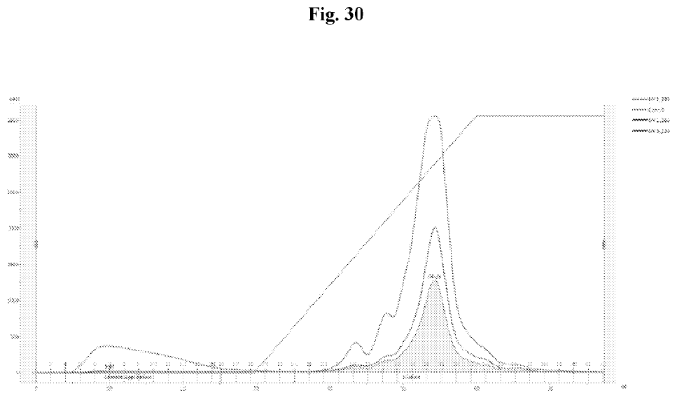

The reaction mixture was purified with an Agilent 1200 using HIC method 2. Fractions containing PMO conjugated to the antibody were combined and concentrated with Amicon Ultra-15 centrifugal filter units with a MWCO of 50 kDa. The conjugate was concentrated and buffer exchanged with PBS (pH 7.4) using Amicon Ultra-15 centrifugal filter units prior to analysis. FIG. 30 shows the SCX chromatogram of the CD71 mAb-PMO purification using SCX method 2.

### Step 3: Analysis of Purified CD71 mAb-PMO

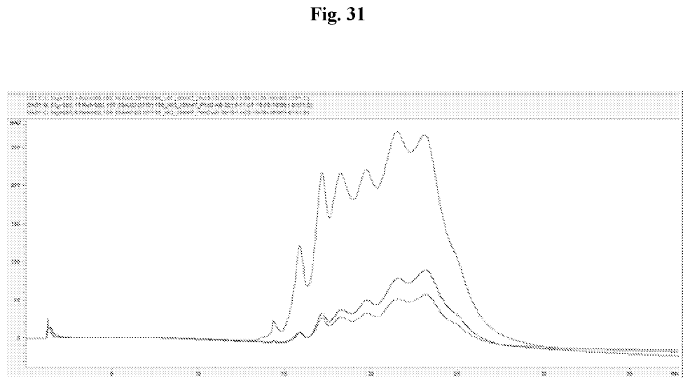

Analysis of the CD71 mAb-PMO was performed with UV-Vis, SCX and SEC. The 260/280 nm UV absorbance ratio of each sample was compared to a standard sample of unreacted Ab. HIC method 1 was used to assess that the average DAR was 4.3 with 0.8% unreacted antibody remaining in the mixture. SEC method 1 was to confirm the absence of high molecular weight aggregates and unconjugated PMO. FIG. 31 shows the analysis of purified low DAR CD71 mAb-PMO using HIC method 1.

### Example 20. Production and Characterization of the CD71 Antibody Morpholino Antisense Oligonucleotide Conjugate (CD71 mAb-PMO) Using a SMCC Linker

### Step 1: CD71 mAb-PMO Conjugation

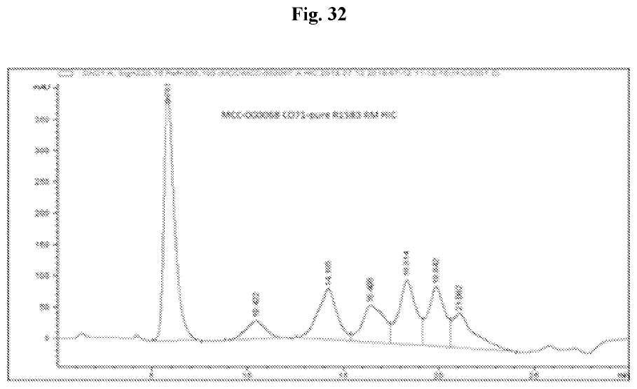

Antibody (6.81 mg/ml) in phosphate buffer pH 7.2 was reduced by adding 6 equivalents of tris(2-carboxyethyl)phosphine (TCEP) in water and incubating at 37° C. for 4 hours. 4(N-Maleimidomethyl)cyclohexanecarboxylic acid N-hydroxysuccinimide ester (SMCC) was coupled to the primary amine on the 3′ end of the PMO by incubating the PMO (15 mg/ml) in 100 mM phosphate buffer pH7.4 and DMSO (1:1) with 9 equivalents of SMCC (30 mg/ml) in DMSO for one hour. Unconjugated SMCC was removed by adding 20 volumes of acetone followed by centrifugation. The pellet was dissolved in 10 mM acetate buffer pH 6.0 then an additional 20 volumes of acetone was added followed by centrifugation. The MCC-PMO pellet was dried at room temperature then dissolved 10 mg/ml in 10 mM acetate buffer pH 6.0. The reduced antibody was mixed with 2.8 equivalents of MCC-PMO and incubated overnight at 4° C. After 16 h, 8 equivalents of N-Ethylmaleimide were added to the mixture at room temperature for 30 minutes to quench unreacted cysteines. Analysis of the reaction mixture by hydrophobic interaction chromatography (HIC) method 3 showed antibody-PMO conjugates along with unreacted antibody and PMO, see FIG. 32. FIG. 32 shows the chromatogram of CD71 mAb-PMO reaction mixture produced with HIC method 3 showing free antibody peak (1), free PMO (2), DAR 1 (3), DAR 2 (4), DAR 3 (5), DAR >3 (6).

### Step 2: Purification

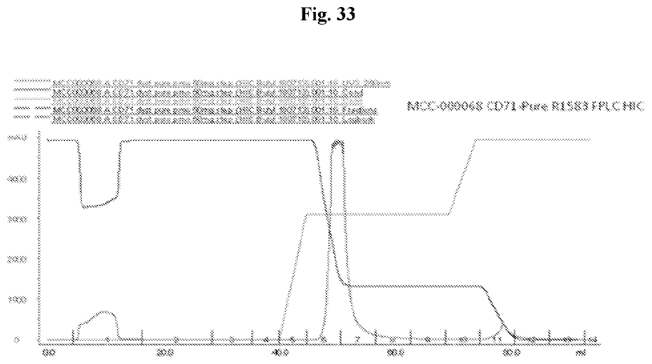

The reaction mixture was purified with an AKTA Explorer FPLC using HIC method 4. Fractions containing conjugates with a drug to antibody of two and greater (DAR≥2) were combined and concentrated with Amicon Ultra-15 centrifugal filter units with a MWCO of 50 kDa. Concentrated conjugates were buffer exchanged with PBS (pH 7.4) using Amicon Ultra-15 centrifugal filter units prior to analysis. FIG. 33 shows the HIC chromatogram of the CD71 mAb-PMO purification using HIC method 4.

### Step 3: Analysis of the Purified Conjugate

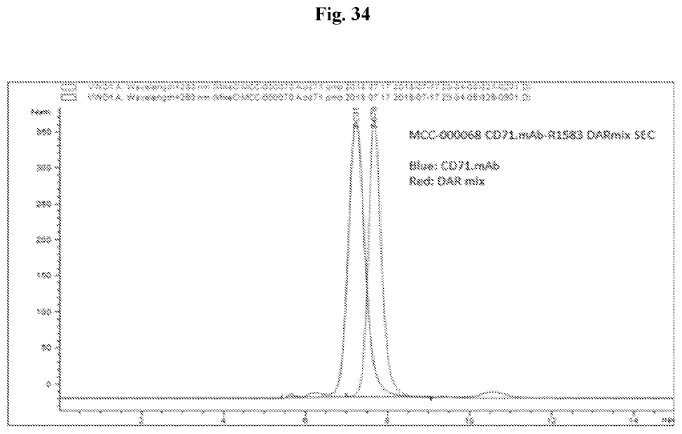

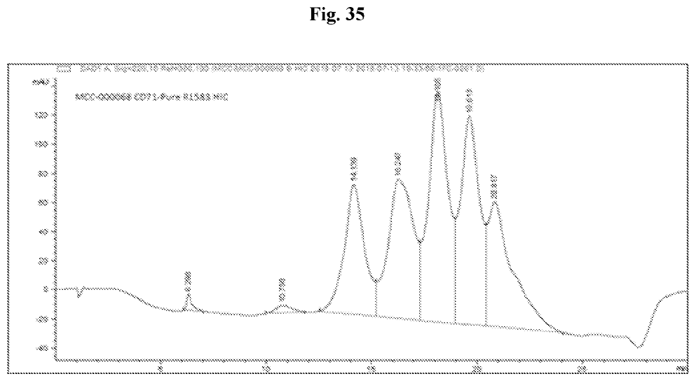

The isolated conjugates were characterized by size exclusion chromatography (SEC), and HIC. SEC method 2 was used to confirm the absence of high molecular weight aggregates and unconjugated PMO, see chromatograms in FIG. 34. The purity of the conjugate was assessed by analytical HPLC using HIC method 4 as can be seen in chromatograms in FIG. 35. FIG. 34 shows the chromatogram of CD71 mAb and CD71-mAb-PMO DAR≥2 produced using SEC method 2. FIG. 35 shows the chromatogram of purified CD71 mAb-PMO DAR≥2 conjugate produced using HIC method 4.

### Example 21. Analytical and Purification Methods

### Reversed-Phase HPLC (RP-HPLC) Method 1

1. Column: Phenomenex Aeris 3.6 μm WIDEPORE XB-C18 250×4.6 mm
2. Column Temperature: 80° C.
3. Solvent A: 0.1% TFA in water
4. Solvent B: 0.1% TFA in acetonitrile
5. Detectors: UV absorbance, 220, 260 and 280 nm
6. Flow Rate: 1 ml/min
7. Gradient:

| | Time | % A | % B |
|---|---|---|---|
| a. | 0.0 | 90 | 10 |
| b. | 1.0 | 85 | 15 |
| c. | 15.0 | 55 | 45 |
| d. | 16.0 | 0 | 100 |
| e. | 21.0 | 0 | 100 |
| f. | 21.5 | 90 | 10 |
| g. | 27.0 | 90 | 10 |

### Strong Cation Exchange Chromatography (SCX) Method 1

1. Column: GE HiPrep SP HP 16/10 20 mL CV
2. Column Temperature: 25° C.
3. Mobile phase A: 20 mM PB pH 7, 25% CAN
4. Mobile phase B: 20 mM PB pH 7, 1.5 M Guanidinium HCl, 25% acetonitrile
5. Flow rate: 3 ml/min
6. Gradient:

| | % B | Column Volume (CV) |
|---|---|---|
| a. | 10 | Load |
| b. | 50 | 0.25 |
| c. | 65 | 2.5 |
| d. | 100 | 0.25 |
| e. | 100 | 1 |

### Strong Cation Exchange Chromatography (SCX) Method 2

1. Column: GE 5 ml SP HP column
2. Column Temperature: 25° C.
3. Mobile phase A: 20 mm NaH2PO4 pH7, 25% ACN
4. Mobile phase B: 20 mm NaH2PO4, 1.5M guanidine HCl, pH7, 25% ACN
5. Detectors: UV absorbance, 220, 260 and 280 nm
6. Flow rate: 0.6 ml/min
7. Gradient:

| | % B | CV |
|---|---|---|
| a. | 50 | 0.25 |
| b. | 65 | 2.5 |
| c. | 100 | 0.25 |
| d. | 100 | 1 |
| e. | 100 | 1 |

### Strong Cation Exchange Chromatography (SCX) Method 3

1. Column: GE HiPrep SP HP 16/10 20 mL CV
2. Column Temperature: 25° C.
3. Mobile phase A: 1×PBS, pH 7.5, 10% ethanol
4. Mobile phase B: 1×PBS, pH 7.5, 10% ethanol, 1.5 M sodium chloride
5. Flow rate: 3 ml/min
6. Gradient:

| | % B | CV |
|---|---|---|
| a. | 10 | 0.5 |
| b. | 100 | 2 |
| c. | 100 | 2 |

### Strong Cation Exchange (SCX) Method 4

1. Column: Thermo Scientific, MabPacSCX-10 column 4×250 mm
2. Solvent A: 20 mM sodium phosphate pH 7, 25% Acetonitrile; Solvent B: 20 mM Sodium phosphate, 1.5M Guanidine HCl pH 7, 25% acetonitrile
3. Flow Rate: 0.65 ml/min
4. Gradient:

| | Time | % A | % B |
|---|---|---|---|
| a. | 0.00 | 100 | 0 |
| b. | 2.00 | 100 | 0 |
| c. | 32.00 | 0 | 100 |
| d. | 32.50 | 0 | 100 |
| e. | 33.00 | 100 | 0 |
| f. | 39.00 | 100 | 0 |

### Strong Cation Exchange (SCX) Method 5

1. Column: GE SP HP Column 4.7 ml
2. Solvent A: 20 mM sodium phosphate, 150 mM sodium chloride pH 7.4, 10% ethanol; Solvent B: 20 mM sodium phosphate, 1.5M sodium chloride pH 7.4, 10% ethanol
3. Flow Rate: 0.6 ml/min
4. Gradient:

| | % A | % B | CV |
|---|---|---|---|
| a. | 100 | 0 | 2 |
| b. | 0 | 50 | 8 |
| c. | 0 | 100 | 0.1 |

### Hydrophobic Interaction Chromatography (HIC) Method 4

1. Column: GE, HiScreen Butyl HP, 4.7 ml
2. Solvent A: 50 mM phosphate buffer, 0.8M Ammonium Sulfate, pH 7.0; Solvent B: 80% 50 mM phosphate buffer, 20% IPA, pH 7.0; Flow Rate: 1.0 ml/min
3. Gradient:

| | % A | % B | CV |
|---|---|---|---|
| a. | 100 | 0 | 4 |
| b. | 40 | 60 | 1 |
| c. | 40 | 60 | 4 |
| d. | 0 | 100 | 1 |
| e. | 0 | 100 | 3 |

### Size Exclusion Chromatography Method 1

1. Column: Phenomenex Yarra, 3 um SEC-3000, 300×7.8 mm
2. Mobile phase: 0.05 M KH2PO4, 0.2 M KCl ddH2O, pH 6.8, 10% isopropanol
3. Flow Rate: 1.0 ml/min for 15 mins
4. Detectors: UV absorbance at 220, 260 and 280 nm.

### Size Exclusion Chromatography (SEC) Method 2

1. Column: TOSOH Biosciences, TSKgelG3000SW XL, 7.8×300 mm, 5 uM
2. Mobile phase: 20 mM phosphate buffer, pH 7
3. Flow Rate: 1.0 ml/min for 20 mins

### Size Exclusion Chromatography (SEC) Method 2

1. Column: TOSOH Biosciences, TSKgelG3000SW, 21.5×600 mm, 5 mM
2. Mobile phase: PBS pH 7.4
3. Flow Rate: 1.0 ml/min for 180 mins

### Hydrophobic Interaction Chromatography (HIC) Method 1

1. Column: Thermo Scientific, MAbPac HIC-Butyl 5 μm, 4.5×100 mm
2. Solvent A: 100 mM phosphate buffer, 1.2 M sodium sulfate pH 7.0;
3. Solvent B: 80% 100 mM phosphate buffer, pH 7.0, 20% isopropanol
4. Flow rate=1 ml/min
5. Sample: dilute sample to 2.5 mg/ml antibody in PBS, inject 10 μl
6. Detectors: FLD excitation 280 nm, emission 345. UV absorbance, 220, 260 and 280 nm.
7. Gradient:

| | Time | % A | % B |
|---|---|---|---|
| a. | 0.0 | 100 | 0 |
| b. | 1.00 | 100 | 0 |
| c. | 1.50 | 100 | 0 |
| d. | 26.50 | 0 | 100 |
| e. | 28.50 | 0 | 100 |
| f. | 29.00 | 100 | 0 |
| g. | 33.00 | 100 | 0 |

### Hydrophobic Interaction Chromatography (HIC) Method 2

1. Column: GE HiScreen Butyl HP 4.7 mL CV
2. Column Temperature: 25° C.
3. Solvent A: 100 mM phosphate buffer, 0.7 M sodium sulfate pH 7.0;
4. Solvent B: 80% 100 mM phosphate buffer, pH 7.0, 20% isopropanol
5. Flow rate: 0.6 ml/min
6. Gradient:

| | % B | CV |
|---|---|---|
| a. | 0 | 1 |
| b. | 100 | 6.5 |
| c. | 100 | 5 |

### Hydrophobic Interaction Chromatography (HIC) Method 3

1. Column: Thermo Scientific, MAbPac HIC-20, 4.6 mm ID×10 cm, 5 μm
2. Solvent A: 100 mM phosphate buffer, 1.8 M Ammonium Sulfate, pH 7.0; Solvent B: 80% 100 mM phosphate buffer, 20% IPA, pH 7.0; Flow Rate: 0.7 ml/min
3. Gradient:

| | Time | % A | % B |
|---|---|---|---|
| a. | 0.00 | 100 | 0 |
| b. | 2.00 | 100 | 0 |
| c. | 22.00 | 0 | 100 |
| d. | 25.00 | 0 | 100 |
| e. | 26.00 | 100 | 0 |
| f. | 30.00 | 100 | 0 |

### Hydrophobic Interaction Chromatography (HIC) Method 4

1. Column: GE, HiScreen Butyl HP, 4.7 ml
2. Solvent A: 50 mM phosphate buffer, 0.8M Ammonium Sulfate, pH 7.0; Solvent B: 80% 50 mM phosphate buffer, 20% IPA, pH 7.0; Flow Rate: 1.0 ml/min
3. Gradient:

| | % A | % B | CV |
|---|---|---|---|
| a. | 100 | 0 | 4 |
| b. | 40 | 60 | 1 |
| c. | 40 | 60 | 4 |
| d. | 0 | 100 | 1 |
| e. | 0 | 100 | 3 |

Examples 22-26 disclose protocols for synthesizing and validating antibody-peptide-oligonucleotide conjugates

### Example 22. Fmoc-(RXR)4XB-mEx23PMO Conjugation

Lyophilized 3′NH2 functionalized mEx23 PMO (sequence: GGCCAAACCTCGGCTTACCTGAAAT (SEQ ID NO:28)) was dissolved in anhydrous DMSO. In a separate vial, the Fmoc-(RXR)4XB peptide (“(RXR)4XB” disclosed as SEQ ID NO: 1065) (4 molar equivalents) and HATU (4 molar equivalents) were dissolved in a mixture of anhydrous DMSO and DIPEA (10 molar equivalents) and let sit for 5 minutes. The PMO solution and the peptide solution were combined and the reaction was left for 1 h at room temperature. The reaction mixture was diluted with nanopure water. The reaction was concentrated in a 15 mL 3,000 MWCO Amicon spin filter (4,000×g for 30 min), diluted to 15 mL with nanopure water, and repeated for a total of 3 washes. The final was not diluted with water. The reaction mixture was purified by SCX purification. The pooled fractions containing product were concentrated in a 15 mL 3,000 MWCO Amicon spin filter (4,000×g for 30 min), diluted to 15 mL with nanopure water, and repeated for a total of 5 washes. The final was not diluted with water. The product was then filtered through a sterile 0.22 um syringe filter and lyophilized in a pre-weighed vial. The product (Fmoc-(RXR)4XB-mEx23PMO) was characterized by RP-HPLC.

### Example 23. Fmoc Deprotection of Fmoc-(RXR)4XB-mEx23PMO

Lyophilized Fmoc-(RXR)4XB-mEx23PMO was dissolved in anhydrous DMSO saturated with piperazine. The solution was let sit at room temperature and monitored by RP-HPLC. When the reaction was completed, the reaction mixture was diluted into nanopure water, concentrated in a 15 mL 3,000 MWCO Amicon spin filter (4,000 xg for 30 min), diluted to 15 mL with nanopure water, and repeated for a total of 5 washes. The final was not diluted with water. The product was then filtered through a sterile 0.22 um syringe filter and lyophilized in a pre-weighed vial. The product (H2N-(RXR)4XB-mEx23PMO) was characterized by RP-HPLC.

### Example 24. H2N-(RXR)4XB-mEx23PMO Conjugation with 6-Maleimidocaproic Acid

Lyophilized H2N-(RXR)4XB-mEx23PMO was dissolved in 50% pH 7.2 phosphate buffer and 50% DMSO. 6-maleimidocaproic acid was dissolved in anhydrous DMSO and added to the H2N-(RXR)4XB-mEx23 PMO solution. The reaction was let sit at room temperature for 30 min. The reaction mixture diluted into pH 6 acetate buffer, concentrated in a 15 mL 3,000 MWCO Amicon spin filter (4,000 xg for 30 min), diluted to 15 mL with pH 6 acetate buffer, and repeated for a total of 5 washes. The final was not diluted with water. The product was then filtered through a sterile 0.22 um syringe filter and stored at −20 C. The product (MC-(RXR)4XB-mEx23PMO) was characterized by RP-HPLC.

### Example 25. MC-(RXR)4XB-mEx23PMO Conjugation with Reduced Anti-mTfR1 Antibody

Anti-mTfR1 antibody solution was combined with ethylenediaminetetraacetic acid solution and tris(2-carboxyethyl)phosphine hydrochloride solution and incubated for 2 hours at 37 C. The reduced antibody was combined with MC-(RXR)4XB-mEx23PMO and let sit at room temperature for 30 min. N-ethylmaleimide solution was added to the reaction mixture to cap any unreacted cystines. The product was purified by cation exchange to remove excess small molecule components. The purified product was buffer exchanged into PBS and stored under refrigeration. The product (anti-mTfR1-MC-(RXR)4XB-mEx23PMO) was characterized by SCX, HIC, SEC, and UV Vis.

### Example 26. In Vitro Detection of DMD Exon Skipping

### Methods for Determining DMD Exon 23 Skipping in Differentiated C2C12 Cells

Mouse myoblast C2C12 cells (ATCC CRL-1772) were plated at 50,000/well in 24-well collagen plates in 1 mL DMEM+10% FBS media and incubated at 37° C. with 5% C02. On the fourth day, cells were switched to differentiation media (DMEM+2% horse serum and 1×ITS, ThermoFisher #41400045) and incubated for 2 days. Following incubation, compounds (Table 23) were added into C2C12 myotubes and incubated for 48 hours. PMO and PPMO transfections were done in the presence and absence of 2 μL of Endo-Porter (Gene Tools, SKU OT-EP-DMSO-1). All the compounds were titrated in a series of 2-fold dilution with differentiation media (Table 24). At 48 hours after treatments, cells were harvested.

TABLE 23
List of compounds tested

| | | | | | Final |
|---|---|---|---|---|---|
| | | PMO | | | PMO |
| Compound | Batch# | uM | PMO | Medium | conc |
| mEx23 PMO | R-1583 | 1000 | 24.2 | 195.8 | 10 uM |
| mEx23-R6 PPMO | MCC-111 | 1000 | 24.2 | 195.8 | 10 uM |
| mEx23-RXR PPMO | MCC-112 | 1000 | 24.2 | 195.8 | 10 uM |
| CD71-mEx23 PMO | MCC-068 | 360 | 67.2 | 152.8 | 10 uM |
| cd71-PPMO R6 DAR1 | MCC-123 A | 30.6 | 197.7 | 22.3 | 2.5 uM |
| cd71-PPMO RXR | MCC-124 A | 97 | 124.7 | 95.3 | 5 uM |
| DAR1 | | | | | |
| cd71-PPMO R6 DAR3 | MCC-123 B | 175 | 138.3 | 81.7 | 10 uM |
| cd71-PPMO RXR | MCC-124 B | 199 | 121.6 | 98.4 | 10 uM |
| DAR3 | | | | | |

TABLE 24
Plate map of in vitro experiment

| +Endo-Porter | | | −Endo-Porter | | | | | | | |
|---|---|---|---|---|---|---|---|---|---|---|
| R-1583 | MCC-111 | MCC-112 | R-1583 | MCC-111 | MCC-112 | MCC-068 | MCC-123A | MCC-124A | MCC-123B | MCC-124B |
| 10 | 10 | 10 | 10 | 10 | 10 | 10 | 2.5 | 5 | 10 | 10 |
| 5 | 5 | 5 | 5 | 5 | 5 | 5 | 1.25 | 2.5 | 5 | 5 |
| 2.5 | 2.5 | 2.5 | 2.5 | 2.5 | 2.5 | 2.5 | 0.63 | 1.25 | 2.5 | 2.5 |
| 1.25 | 1.25 | 1.25 | 1.25 | 1.25 | 1.25 | 1.25 | 0.31 | 0.63 | 1.25 | 1.25 |
| 0.63 | 0.63 | 0.63 | 0.63 | 0.63 | 0.63 | 0.63 | 0.16 | 0.31 | 0.63 | 0.63 |
| 0.31 | 0.31 | 0.31 | 0.31 | 0.31 | 0.31 | 0.31 | 0.08 | 0.16 | 0.31 | 0.31 |
| 0.16 | 0.16 | 0.16 | 0.16 | 0.16 | 0.16 | 0.16 | 0.04 | 0.08 | 0.16 | 0.16 |
| 0 | 0 | 0 | 0 | 0 | 0 | 0 | 0 | 0 | 0 | 0 |

| | |
|---|---|
| PMO | AOC |

RNAs were isolated using Zymo Direct-zol-96 RNA Kit (Zymo Research; R2056) and reverse transcribed using High Capacity cDNA Reverse transcription Kit (ThermoFisher #4368813). Real-time PCR reactions were performed using Taqman Fast Advanced Master Mix (Thermo Fisher #4444558). Taqman real-time PCR primer/probe sets used were listed in Table 25.

TABLE 25
PCR primer sequences (SEQ ID NOS 34-36 and
1068-1070, respectively, in order of
appearance)
Skipped (d23)

| | | |
|---|---|---|
| Forward Primer | mDMD-E22F | GCGCTATCAGGAGACAATGAG |
| Reverse Primer | mDMD-E22- | GTTTTTATGTGATTCTGTAA |
| | 24R | TTTCCC |
| Probe | | CTCTCTGTACCTTATCTTAG |
| | | TGTT |

Non-Skipped (Ex22-23)

| | | |
|---|---|---|
| Forward Primer | mDMD-E21F | TCTGGATGCAGACTTTGTGG |
| Reverse Primer | mDMD-E21- | GTTTTTATGTGATTTTGTCT |
| | 24R | GTAGCTC |
| Probe | | TAACCACATCTTTGATGGTG |
| | | TGA |

### Results

FIG. 36 shows Exon skipping (% of total dystrophin RNA) of exon 23 in mouse dystrophin vs treatment PMO concentration for C12C12 cells treated with PMO, PPMO, PMO-AOC or PPMO-AOC. Top left: graph with overlay of all compounds tested. Bottom left: PMO and PPMO samples (=/− endoporter). Bottom right: PMO-AOC and PPMO-AOC samples.

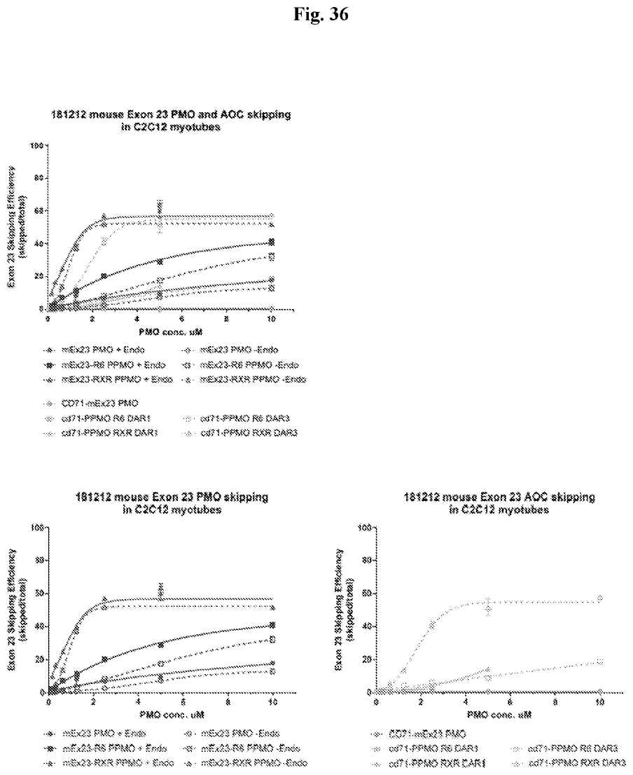

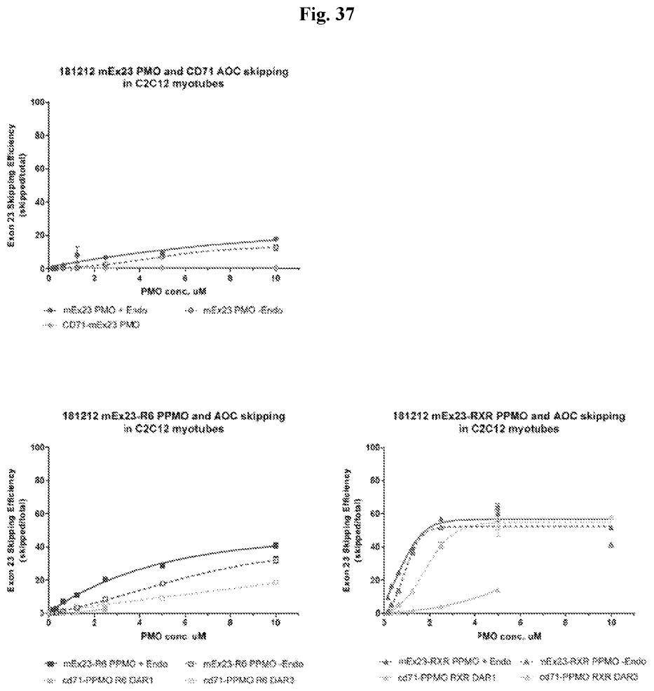

FIG. 37 shows Exon skipping (% of total dystrophin RNA) of exon 23 in mouse dystrophin vs treatment PMO concentration for C12C12 cells treated with PMO, PPMO, PMO-AOC or PPMO-AOC. Top left: graph with overlay of PMO (+/−endoporter) and PMO-AOC. Bottom left: PPMO (R6 peptide (SEQ ID NO: 1072))+/−endoporter and PPMO-AOCs (R6 peptide (SEQ ID NO: 1072)). Bottom right: PMO (RXR4XB peptide (SEQ ID NO: 1065))+/−endoporter and PPMO-AOCs (RXR4XB peptide (SEQ ID NO: 1065))

### Example 27. In Vivo Activity of PMO, PPMO, PMO-AOCs, and PPMO-AOCs

### In Vivo Study and Sample Collection

Animals were dosed intravenously with test articles. All groups contained 5 animals, except for group 12 which only contained 3 animals due to insufficient dosing material. For dosing details, see Table 26 and Table 27.

TABLE 26
Summary of the design of the in vivo study to compare the efficacy of PMO, PPMO, PMO-AOC, and PPMO-AOC compounds

| Study Design: | | | | | | | | | | | |
|---|---|---|---|---|---|---|---|---|---|---|---|
| Animal and Croup Info | | | | | Compound Info | | | | | | |
| | | | | Animal | | | | PMO | Total PMO | PMO:mAb | |
| | | | | Weight | PMO | AOC | Note | Dose | Dose | Ratio | Ab Dose |
| Group | Test Article | Payload | N | (kg) | R# | R# | book | (mg/ml) | (mg/kg) | (mol/mol) | (mg/kg) |
| 1 | PBS (mdx) | N/A | 5 | 0.030 | N/A | N/A | N/A | — | — | — | — |
| 2 | PMO (mdx) | exon 23 PMO | 5 | 0.030 | | | | 2 | 10 | — | — |
| 3 | PPMO (mdx) | exon 23 PPMO | 5 | 0.030 | | | | 2 | 10 | — | — |
| 4 | CD71-PMO DAR | exon 23 PMO | 5 | 0.030 | | | | 0.3 | 1.5 | 1.7 | 16.5 |
| 5 | 1.7 (mdx) | exon 23 PMO | 5 | 0.030 | | | | 0.9 | 4.5 | 1.7 | 50.0 |
| 6 | CD71-PMO DAR | exon 23 PMO | 5 | 0.030 | | | | 0.66 | 3.3 | 3.5 | 16.5 |
| 7 | 3.5 (mdx) | exon 23 PMO | 5 | 0.030 | | | | 2 | 10 | 3.5 | 50.0 |
| 8 | CD71-PPMO DAR | exon 23 PPMO | 5 | 0.030 | | | | 0.3 | 1.5 | 1.7 | 16.5 |
| 9 | 1.7 (mdx) | exon 23 PPMO | 5 | 0.030 | | | | 0.9 | 4.5 | 1.7 | 50.0 |
| 10 | CD71-PPMO DAR | exon 23 PPMO | 5 | 0.030 | | | | 0.66 | 3.3 | 3.5 | 16.5 |
| 11 | 3.5 (mdx) | exon 23 PPMO | 5 | 0.030 | | | | 2 | 10 | 3.5 | 50.0 |
| 12 | Isotype mAb-PPMO | exon 23 PPMO | 3 | 0.030 | | | | 2 | 10 | 3.5 | 50.0 |
| | DAR 3.5 (mdx) | | | | | | | | | | |
| 14 | PBS (C57BL/6) | | 5 | 0.030 | N/A | N/A | N/A | | | — | — |
| | | | 58 | mdx | | | | | | | |

TABLE 27
Summary of the design of the in vivo study to compare the efficacy of PMO, PPMO, PMO-AOC, and PPMO-AOC compounds

| | | | | | | | | | Overage | | | | | | | |
|---|---|---|---|---|---|---|---|---|---|---|---|---|---|---|---|---|
| | | | | | | | | | (ml); | 0.1 | | | | | | |
| | | | | | Dosing Info | | | | | | | | | | | |
| Study Design: | | | | | | | | | Inj | Inj | | | | | | |
| Animal and Group info | | | | | | | | | Vol | Vol | Sample | | | | | |
| | | | | Animal | | Dose | | | per | per | Collection | | | Materials | | |
| | | | | Weight | | Volume | # of | Dose | Animal | Group | Endpoints | | | | | CD71 |
| Group | Test Article | Payload | N | (kg) | ROA | (kg) | Doses | Schedule | (mL) | (mL) | [symbol] | [symbol] | [symbol] | PMO | PPMO | mAb |

| | | | | | | | | | | | | | |
|---|---|---|---|---|---|---|---|---|---|---|---|---|---|
| 1 | PBS (mdx) | NA | 5 | 0.030 | IV | 5 | 1 | t = 0 | 0.15 | 0.85 | — | — | 14 |
| 2 | PMO (mdx) | exon 23 | 5 | 0.030 | IV | 5 | 1 | t = 0 | 0.15 | 0.85 | — | — | 14 |
| | | PMO | | | | | | | | | | | |
| 3 | PPMO (mdx) | exon 23 | 5 | 0.030 | IV | 5 | 1 | t = 0 | 0.15 | 0.85 | — | — | 14 |
| | | PPMO | | | | | | | | | | | |
| 4 | CD71 PMO | exon 23 | 5 | 0.030 | IV | 5 | 1 | t = 0 | 0.15 | 0.85 | — | — | 14 |
| | DAR 1.7 | PMO | | | | | | | | | | | |
| 5 | (mdx) | exon 23 | 5 | 0.030 | IV | 5 | 1 | t = 0 | 0.15 | 0.85 | — | — | 14 |
| | | PMO | | | | | | | | | | | |
| 6 | CD71 PMO | exon 23 | 5 | 0.030 | IV | 5 | 1 | t = 0 | 0.15 | 0.85 | — | — | 14 |
| | DAR 3.5 | PMO | | | | | | | | | | | |
| 7 | (mdx) | exon 23 | 5 | 0.030 | IV | 5 | 1 | t = 0 | 0.15 | 0.85 | — | — | 14 |
| | | PMO | | | | | | | | | | | |
| 8 | CD71 PPMO | exon 23 | 0 | 0.030 | IV | 5 | 1 | t = 0 | 0.15 | 0.85 | — | — | 14 |
| | DAR 1.7 | PPMO | | | | | | | | | | | |
| 9 | (mdx) | exon 23 | 5 | 0.030 | IV | 5 | 1 | t = 0 | 0.15 | 0.85 | — | — | 14 |
| | | PPMO | | | | | | | | | | | |
| 10 | CD71 PPMO | exon 23 | 5 | 0.030 | IV | 5 | 1 | t = 0 | 0.15 | 0.85 | — | — | 14 |
| | DAR 3.5 | PPMO | | | | | | | | | | | |
| 11 | (mdx) | exon 23 | 5 | 0.030 | IV | 5 | 1 | t = 0 | 0.15 | 0.85 | — | — | 14 |
| | | PPMO | | | | | | | | | | | |
| 12 | Isotype mAb- | exon 23 | 3 | 0.030 | IV | 5 | 1 | t = 0 | 0.15 | 0.85 | — | — | 14 |
| | PPMO DAR | PPMO | | | | | | | | | | | |
| | 3.5 (mdx) | | | | | | | | | | | | |
| 14 | PBS | | 5 | 0.030 | IV | 5 | 1 | t = 0 | 0.15 | 0.85 | — | — | 14 |
| | (C57BL/6) | | 58 | mdx | | | | | | | | | |
| | | | 5 | WT | | | | | | | | | |

[symbol]

indicates data missing or illegible when filed

Animals were harvested 14 days post dose, except for animals in group 11, which were mistakenly taken down 120 hours post dose. One animal from group 5 was taken down early due to hunched posture, low body weight, and low skin turgor and discarded from analysis. One animal in group 8 was found dead before day 14 and discarded from analysis.

Two 20-30 mg tissue samples of gastroc, tibialis anterior (TA), liver, diaphragm, and heart were excised from each animal in all groups and stored in collection tubes with a 5 mm steel bead on 96 well plates at −80° C. for subsequent exon skipping analysis and PMO/PPMO tissue concentration analysis. 50 mg tissue samples of gastroc and heart were excised from 3 animals in groups 1, 2, 3, 6, 10, 12, and 13 and stored in cryo bags at −80° C. for future Western blot analysis.

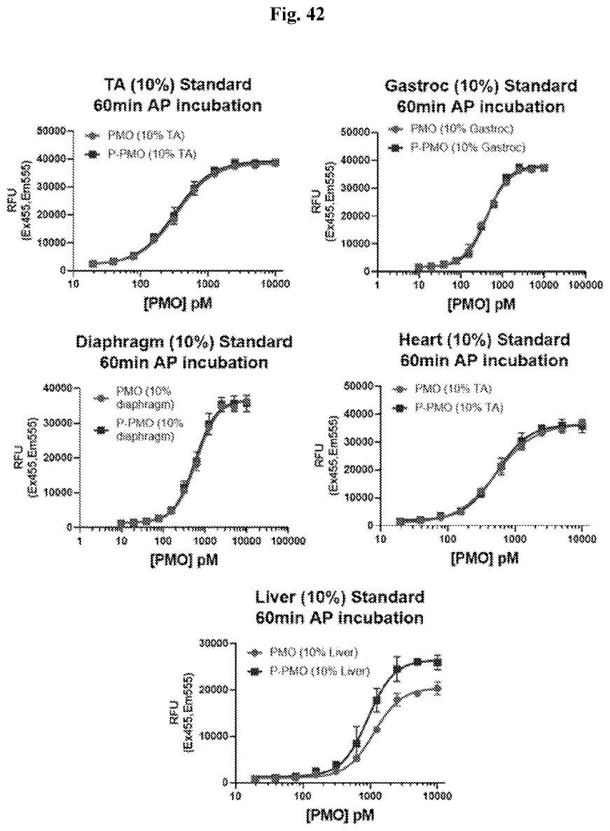

Additional tissue samples of gastroc were excised from 2 animals in groups 1,2,3,6,10,12, and 13, embedded in OCT, and frozen at −80° C. for future dystrophin IHC analysis at REVEAL. For exact tissue weights of each sample, see FIG. 42.

### Exon Skipping Sample Homogenization

Tissue samples for exon skipping analysis were removed from −80° C. and allowed to thaw at room temperature for approximately 10 minutes. Foil covering was carefully removed from collection tubes. Any samples stuck near the tops of the collection tubes were poked down with individual pipette tips.
500 μL of Trizol was added to each 20-30 mg tissue sample. Tubes were capped tightly.
Plates were homogenized on the Tissue Lyser II for 5 minutes at 30 Hz and then cooled on dry ice for approximately 3 minutes. Plates were homogenized in the opposite orientation for another 5 minutes at 30 Hz and then cooled on dry ice for approximately 3 minutes. Samples were inspected for thorough homogenization. If samples were not thoroughly homogenized, a third round of homogenization was performed for 3 minutes at 30 Hz, and then samples were cooled on dry ice for 3 minutes.
After complete homogenization, plates were centrifuged for 5 minutes at 6,000 RCF to pull any large tissue pieces to the bottom of the tube.
50 μL of tissue homogenate from each sample was transferred to a well on a 96 well plate. Tubes were capped and stored at −80° C. for future use if necessary.

### Exon Skipping Sample RNA Isolation

RNA from exon skipping samples was isolated using the Zymo Research Direct-zol-96 RNA Kit (Zymo Research; R2056). An equal amount (50 uL) of 100% ethanol was added to the tissue homogenate. Ethanol and tissue homogenate were mixed thoroughly by pipetting up and down approximately 20 times.
All 100 μL of the mixture was transferred to a well on the Zymo-Spin I-96 plate mounted on top of a collection plate. The spin column plate was covered with a foil seal and centrifuged for 5 minutes at 6,000 RCF. Flow through was discarded and the spin column plate was placed on top of a new collection plate.
400 uL RNA Wash Buffer was added to each well of the spin plate. The spin column plate was covered with a foil seal and centrifuged for 5 minutes at 6,000 RCF. Flow through was discarded.
5 μL of DNase1 and 35 μL of Digestion Buffer were added to the center of each well. The plate was incubated at room temperature for 15 minutes.
400 ul of Direct-zol RNA Pre Wash was added to each well. The spin column plate was covered with a foil seal and centrifuged for 5 minutes at 6,000 RCF. Flow through was discarded.
Previous step was repeated.
800 μL of RNA Wash Buffer was added to each well. Spin column plate was covered with a foil seal and centrifuged for 5 minutes at 6,000 RCF. Flow through was discarded.
Plates were centrifuged again for 5 minutes at 6,000 RCF to remove any remaining RNA Wash Buffer from previous steps.
A new 96 well PCR plate was sandwiched between the collection plate and spin column plate. 26 μL of DNase/RNase free water was added to the center of each well of the spin column plate. Plates were covered with a foil seal and centrifuged for 5 minutes at 6,000 RCF. The 96 well plate containing the eluted RNA was covered with a foil seal and stored at −80° C.

### Quantification and Normalization of RNA

RNA plates were removed from −80° C. and allowed to thaw on ice.
Plates were briefly centrifuged, mixed on shakers (two rounds of approximately 1500 RPM for 30 seconds), and briefly centrifuged again.
RNA concentration was quantified using the Tecan Infinite F200 Microplate Reader and the Tecan NanoQuant Plate. Tecan Infinite F200 Microplate Reader was blanked with nuclease free water. 2 μL of RNA from each sample were used for quantification.
All samples were normalized to 20 ng RNA/uL in nuclease free water, unless the RNA concentration of a sample happened to be <20 ng RNA/uL.

### Reverse Transcription

Normalized RNA (20 ng/uL) was converted to cDNA through reverse transcription (RT).
A master mix was prepared using the High-Capacity cDNA Reverse Transcription Kit (Thermo Fisher; #4368813) and Nuclease free water following the Table 28 below.

TABLE 28
Reverse transcription reagent

| Reagent | 1 reaction (uL) |
|---|---|
| 10X RT Buffer | 2 |
| 25X dNTP | 0.8 |
| 10X Random Primer | 2 |
| RT Enzyme | 1 |
| Nuclease Free Water | 4.2 |
| Total | 10 |

10 μL of master mix was added to each well of a 96 well PCR plate.
10 μL of RNA (20 ng/uL) was added to each well, for a total volume of 20 uL in each well and a total input of 200 ng RNA in each well.
PCR plates were sealed with optical adhesive film, briefly centrifuged, mixed on a shaker (two rounds at approximately 1500 RPM for 30 seconds), and briefly centrifuged again.
PCR plates were placed in a thermocycler and programmed to incubate at 25° C. for 10 minutes, 37° C. for 120 minutes, 85° C. for 5 minutes, and then held at 4° C.
After completion of RT, plates were briefly centrifuged. cDNA was diluted 1:1 with water (20 uL of cDNA diluted with 20 μL of water). Plates were covered with a foil seal and stored at −20° C. until further use.
   qPCR for Exon Skipping Analysis
Exon skipping was analyzed via qPCR using Tagman assays to detect skipped and non-skipped transcripts. PPIB was also included in the analysis for normalization. (At this time, we were not yet routinely doing exon skipping analysis with a Tagman assay to detect Total DMD.) Samples were ran in duplicate.
A qPCR master mix was prepared following the Table 29 below.

TABLE 29
qPCR mix

| Reagent | 1 reaction (uL) |
|---|---|
| Target Probe | 0.17 |
| Skipped: “d23”, FAM, 60X -or- | |
| Non-Skipped: “Ex22-23”, FAM, 60X | |
| PPIB Probe, VIC, 60X | 0.17 |
| Water | 0.66 |
| PCR Fast Master Mix | 5 |
| Total | 6 |

6 μL of master mix was added to each well of a 384 well qPCR plate.
4 μL of cDNA (diluted 1:1 with water) was added to each well, for a total qPCR reaction volume of 10 uL.
Plates were sealed with clear optical adhesive film. Plates were briefly centrifuged, mixed on a plate shaker (two rounds of approximately 2,000 RPM for 30 seconds), and briefly centrifuged again.
Plates were placed into the Applied Biosystems QuantStudio 6/7 Flex Real-Time PCR System, experimental properties were defined, and the following settings were applied:
   Polymerase activation at 95 C for 20 seconds
   PCR 40 cycles
      Denature at 95° C. for 1 second
      Anneal/extend at 60° C. for 20 seconds.
For this study, qPCR thresholds for all targets were set to 0.2.
Data was exported into excel. Exon skipping was calculated as

$$\% skip = \frac{{100}^{*} [ {100}^{*} 2 \hat{} ( Δ ( {Ct}_{non skipped} - {CT}_{PPIB} ) - Δ ( {CT}_{skipped} - {CT}_{PPIB} ) ]}{100 + [ {100}^{*} 2 \hat{} ( Δ ( {Ct}_{non skipped} - {CT}_{PPIB} ) - Δ ( {CT}_{skipped} - {CT}_{PPIB} ) ]}$$

### Exon Skipping Analysis Results

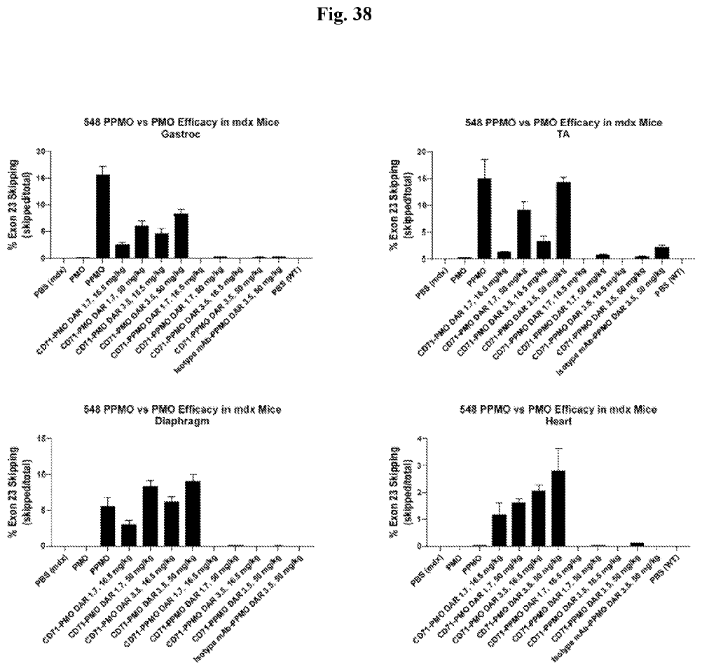

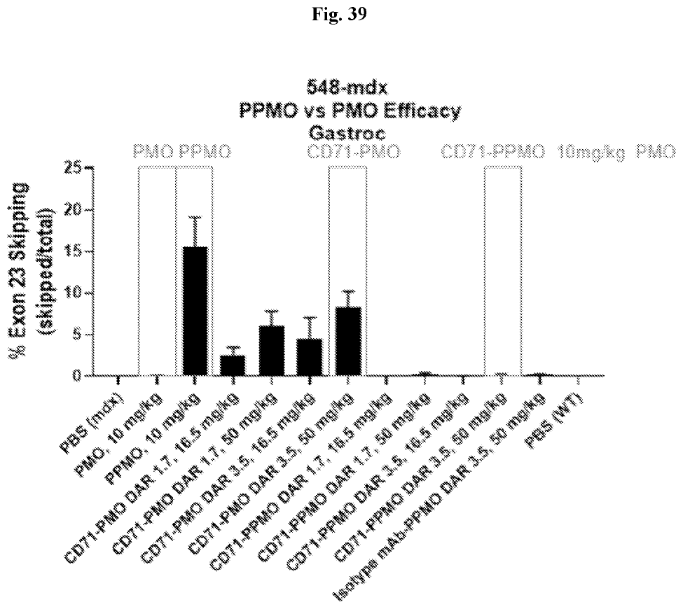

Overall, exon skipping levels were higher in gastroc, TA, and diaphragm than in the heart (FIG. 38). Free PMOs did not show any exon 23 skipping activity in any of the four tissues analyzed. Free PPMOs were active in the gastroc, TA, and diaphragm, but not in the heart. PMO conjugates showed greater activity than free PMOs (FIGS. 38 and 39). PPMO conjugates lost all activity relative to the PMO conjugates and the free PPMOs. FIG. 38 shows exon Skipping in gastroc, TA, diaphragm, and heart at 14 days post dose. Note: CD-71 PPMO DAR 3.5, 50 mg/kg AB dose group was mistakenly taken down at 120 hours post dose. FIG. 39 shows exon Skipping in gastroc 14 Days post dose. Note: CD-71 PPMO DAR 3.5, 50 mg/kg AB dose group was mistakenly taken down at 120 hours post dose.

PMO/PPMO Tissue Concentration Sample Homogenization
Frozen tissue samples of 25-30 mg in 96-well homogenization tubes with 5 mm steel beads were allowed to thaw at RT for 15 minutes. Aluminum plate sealer was carefully removed, ensuring no tissue fragments were adhered.
400 μL of RIPA buffer was added to each well, then tubes were sealed with caps.
Samples were homogenized on TissueLyser II for 5 minutes at 30/s. The plate was rotated 180 degrees then homogenized again for 5 minutes.
The plate was centrifuged at 6000 rpm for 5 minutes. Tubes were inspected to determine if tissue homogenization was complete. An additional 5 minutes of homogenization was performed when needed.
After successful homogenization and centrifugation, caps were removed then 11 μL of Proteinase K (Promega MC5008) diluted to 8 mg/mL in RIPA buffer was added to each tube. Resulting working concentration was ˜200 μg/mL (varies slightly with respect to tissue concentration; for very large samples, PK concentration can be increased).
Tubes were re-capped. Plates were inverted several times to mix PK into homogenate.
Caps were secured by sandwiching a 96 well PCR plate between the tube caps and the lid of the plate and reinforcing with tape or a heavy weight. Samples were then incubated at 60° C. in oven. After 30 min, the plate was checked to ensure caps were still in place as high heat commonly leads to increased pressure and cap popping. Popped caps were re-secured. The plate was checked once more after 30 minutes, followed by overnight incubation.
The next day, plates were brought to room temperature and then tubes were inspected for sample digestion.
One final 3 minutes of homogenization at 30/s was performed to mix digested samples thoroughly followed by 5 minutes of centrifugation at 6000 rpm.
Caps were removed and sample supernatant was removed by pipetting midway into the tube, taking care to avoid touching the bottom. Samples were diluted into dilution buffer as needed.
Once necessary samples were removed, tubes were re-capped and the plate was stored at −20° C. For subsequent experimentation, the plate was removed from −20° C., heated at 60° C. for >1 hr, cooled to RT, then mixed and centrifuged as previously described.

### PMO/PPMO Tissue Concentration ELISA

### Buffers

Wash buffer: standard 1×TBST (unfiltered) on plate washer system stored at room temperature.
Dilute 10×TBST (Cell Signaling Technology #9997) to 1×TBST in MiliQ deionized water.
Dilution buffer: standard 1×TBST (filtered) with the addition of control muscle lysate or serum as required.
Hybridization buffer: 50 mM Tris-HCl, 1.15 M sodium chloride, pH 7.6, 0.3 mg/mL BSA, 0.1% v/v Triton X-100.
   Make 50 mM Tris-HCl, 1.15 M sodium chloride, pH 7.6. Filter and store at room temperature.
   Add 0.3 mg/mL BSA and 0.1% v/v Triton X-100 on day of experiment.
MNase buffer: 50 mM Tris-HCl (pH 8.5), 200 mM NaCl, 5 mM CaCl₂), 0.1 mg/mL BSA.
   Make 50 mM Tris-HCl (pH 8.5), 200 mM NaCl, 5 mM CaCl₂). Filter and store at room temperature. 10× stocks can be kept at −20 C and diluted to 1× with miliQ deionized water.
   Add 0.1 mg/mL BSA to BSA-free stock on day of experiment.
Detection Antibody Buffer: SuperBlock Blocking Buffer in TBS (Thermo 37535)+0.25% v/v Tween-20; prepared fresh on day of experiment and kept at room temperature.
BSA 10× stock: 10 mg/mL BSA protein (Sigma A2934) dissolved in water was aliquoted and kept at −20 C. Free of preservatives. Kept at 4° C. once thawed.

### Plate Coating

Neutravidin coating buffer (Sigma C3041): the contents of one capsule were emptied into 100 mL MilliQ deionized water; 0.05 M carbonate-bicarbonate, pH 9.6.
Coating buffer should be used within 7 days.
NeutrAvidin Protein (Thermo 31000): 10 mg vial was dissolved in 10 mL DNase-/RNase-free (1 mg/mL) deionized water and stored at 4° C. protected from light.
Grenier Bio-One 96 well, F-bottom (Chimney Well), Black, FLUOTRAC, High Binding, Sterile plates (#655077)
Neutravidin protein at 1 mg/mL was diluted 1:1000 in coating buffer to yield 1 ug/mL working concentration (e.g. 11 uL NA in 11 mL buffer per plate).
100 uL was added to each well of the plate, which was then sealed and incubated at 37° C. for 2 hours.
   Up to 4 hr is acceptable; assay sensitivity is lost if plates are stored at 4° C.

### Standard Curves

For 10× standard curves, 1 uM (1,000 nM) stock of Gene Tools mouse Exon 23 PMO/PPMO was heated at 70 C for 7 minutes, mixed well by pipetting, and diluted in dilution buffer starting at 100 nM (10×) followed by a 1:2 serial dilution for 12 points total.
   10× standard curve=100, 50, 2.5 . . . nM
   After adding 1:10 to appropriate matrix, 1× standard curve=10, 5, 2.5 . . . nM
Digested control plasma or homogenate for each tissue type was used to prepare the standard curve in the same percentage of plasma or homogenate as diluted samples (FIG. 42).
   E.g., for 1:100 samples, 1.11% homogenate in dilution buffer was prepared, then 10× standards were added 1:10, resulting in 1× standard and 1% homogenate in dilution buffer. 1:20 á 5.55% homogenate, 1:10 á 11.11% homogenate, etc.

### Hybridization

Complimentary DNA probe (custom from Eurofins) specific to mouse Exon 23 dystrophin (target) PMO/PPMO was diluted from 1 uM stock to 0.5 nM in hybridization buffer.
Equal volumes of PMO/PPMO standards or diluted experimental samples and 0.5 nM DNA probe solution were mixed in a PCR or deep well plate, sealed, then incubated at 37 C for 30 minutes.
   E.g., 75 uL sample dilutions+75 uL 0.5 nM probe in 96-well PCR plate.
   100 μL of hybridized product was subsequently used in the assay.

### Probe-Plate Binding

After 2 hours at 37° C., Neutravidin solution was emptied from the plate followed by blotting on paper towels.
Wells were washed 3× with 200 μL of 1×TBST on plate washer (pre-set PMO ELISA protocol), followed by blotting on paper towels.
200 μL of SuperBlock Blocking Buffer in TBS (Thermo 37535) was added to wells for 5 minutes, followed by emptying and blotting on paper towels.
100 μL of hybridized PMO/PPMO-DNA probe standards or samples were added to wells.
Plates were sealed with plastic film then incubated at 37° C. for 30 minutes.

### MNase DNA Cleavage

Micrococcal Nuclease (Thermo ENO181). Stock vials stored at −20° C.
Each well requires 150 μL of MNase; through experimentation, it was found that 0.9 U/well was an effective quantity; (0.9 U/well)/(150 uL/well)=0.006 U/uL=6 U/mL working concentration.
For each plate: (16 mL)*(6 U/mL)/(300 U/uL)=0.32 uL of 300 U/uL stock.
i.e. 0.32 uL of 300 U/uL MNase in 16 mL MNase buffer per plate.
After 30 minutes at 37° C., hybridized sample solutions were emptied from the plate followed by blotting on paper towels.
Wells were washed 3× with 200 μL of 1×TBST on plate washer (pre-set PMO ELISA protocol) followed by blotting on paper towels.
150 μL of MNase solution was added to wells.
Plates were sealed with plastic film and then incubated at 37° C. for 1 hour.
Attophos AP Fluorescent Substrate System (Promega S1000) was warmed at 37° C. to be used later in the assay.

### Detection Antibody Binding

Monoclonal Anti-Digoxin-Alkaline Phosphatase antibody (Sigma A1054) was diluted 1:10,000 in detection antibody buffer (SuperBlock Blocking Buffer in TBS (Thermo 37535)+0.25% Tween-20).
For each plate: 1.1 uL Ab in 11 mL buffer.
After 1 hour at 37° C., MNase solution was emptied from the plate followed by blotting on paper towels.
Wells were washed 3× with 200 μL of 1×TBST on plate washer (pre-set PMO ELISA protocol), followed by blotting on paper towels.
100 μL of detection antibody solution was added to wells.
Plates were sealed with plastic film then incubated at 37° C. for 30 minutes.

### Alkaline Phosphatase Assay

AttoPhos AP Fluorescent Substrate System (Promega S1000).
60 mL of kit buffer was added to vial containing 36 mg of substrate and allowed at least 1 hour to dissolve; stored at 4° C. protected from light.
The reagent was warmed to 37 C in an incubator in a previous step.
After 30 minutes at 37° C., detection antibody solutions were emptied from the plate followed by blotting on paper towels.
Wells were washed twice with 3×250 μL of 1×TBST on plate washer (Run PMO ELISA Final Wash protocol two times).
100 μL of AttoPhos substrate solution was added to wells.
   Addition was staggered by 3 minutes between plates to allow for handling and read time.
Plates were returned to the oven at 37° C. (unsealed) and a stopwatch was started.
Plates were read on a Tecan Infinite F200 Microplate Reader
   Fluorescent Endpoint read with Ex=444 and Em=555 and AutoCutoff=550.
   COR96fb clear bottom; 30 flashes/read; Read from bottom; AttoPhos; gain 30; Z position 16100
   Read 60 minutes after addition of AttoPhos.

### Data Analysis for Standard Curves

Collected data were copied and pasted into Excel.
Results for standard curves were copied into GraphPad Prism. X values were transformed using X=log(X). Nonlinear regression analysis with 4PL fit of the semi-log plot was performed. In nearly all instances, R2 >0.99 for this fit. Low and high standard values were inspected to assess plateauing on either end of the curve.

### Data Analysis for Tissue Samples

RFU values for samples were copied into Prism below standard values and interpolation was performed. Values outside of the low and high plateau points were excluded.
Interpolated values were transformed using X=10\hat{ }X to yield ELISA [PMO] pM.
ELISA [PMO] pM was multiplied by the appropriate dilution factor to yield Sample [PMO] pM.
Sample [PMO] pM was multiplied by (400+11+tissue weight)/(tissue weight) to yield Tissue [PMO] pM.
   400 for RIPA volume, 11 for Proteinase K volume, and tissue weight assuming 1 mg/uL density.
Tissue [PMO] pM was divided by 1000 to yield Tissue [PMO] nM.

### PMO/PPMO Tissue Concentration Results

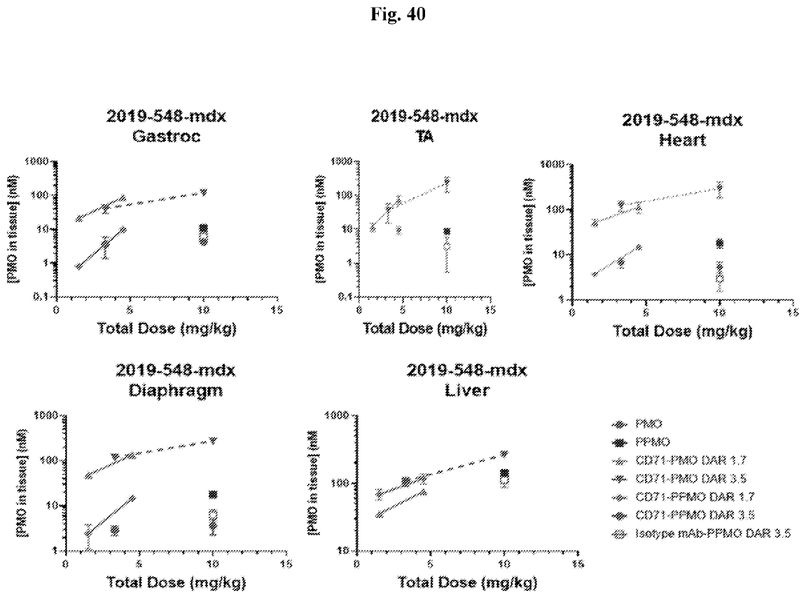

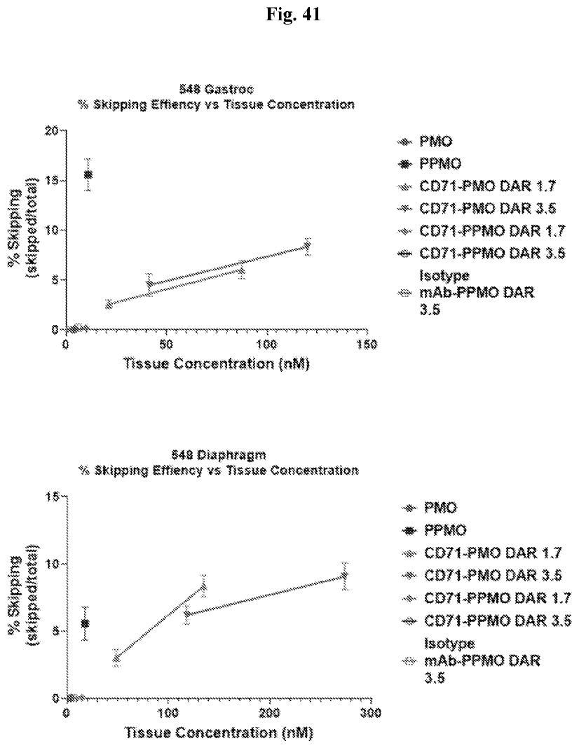

Tissue concentrations of PMOs and PPMOs in samples were interpolated from PMO/PPMO standard curves ran in parallel (FIG. 42). PMOs were not detected in the TA or Liver 14 days post dose (FIG. 40). A further kinetics study should be conducted to understand the kinetics of PMOs at earlier time points. Free PPMOs showed greater uptake into tissue than Free PMOs. PMO conjugates are found in higher concentrations than free PPMOs. However, PMO conjugates yielded less exon skipping activity than free PPMOs despite being present at higher concentrations (FIG. 38 and FIG. 40). PPMO conjugates were detectable in all tissues but PPMO conjugates did now show any exon skipping activity (FIG. 40 and FIG. 41). FIG. 40 shows PMO/PPMO Tissue Concentrations in gastroc, TA, heart, diaphragm, and liver 14 days post dose. Note: CD-71 PPMO DAR 3.5, 50 mg/kg AB dose group was mistakenly taken down at 120 hours post dose and not included in the tissue concentration analysis.

### Exon Skipping Efficiency

Although PMO conjugates are present in gastroc, TA, and diaphragm at higher concentrations than free PPMOs, they tend to show less exon skipping activity (FIG. 41). Free PPMOs seem to be more efficient at exon skipping than PMO conjugates in gastroc, TA, and diaphragm. As previously mentioned, only PMO conjugates showed exon skipping activity in the heart (FIG. 38 and FIG. 41). FIG. 41 shows the exon skipping efficiency. Group average exon 23 skipping (%) is plotted on the y-axis, while group average tissue concentration (nM) is plotted on the x-axis. Note: CD-71 PPMO DAR 3.5, 50 mg/kg AB dose group was mistakenly taken down at 120 hours post dose and not included in the tissue concentration analysis.

### Supporting Data for the In Vivo Studies

Table 30 shows tissue samples and weights. Sample #1 was used for exon skipping analysis. Sample #2 was used for PMO/PPMO tissue concentration analysis. Samples for Western blot analysis appeared to be less than the specified 50 mg. Tissue samples taken for dystrophin IHC analysis with REVEAL were not weighed.

TABLE 30
Tissue and sample weights

| | | | Gastroc | Gastroc | Gastroc - | TA | TA | Liver | Liver | Diaphragm | Diaphragm | Heart | Heart | Heart - |
|---|---|---|---|---|---|---|---|---|---|---|---|---|---|---|
| | Test | | 1 | 2 | WB | 1 | 2 | 1 | 2 | 1 | 2 | 1 | 2 | WB |
| Group | Article | N | (mg) | (mg) | (mg) | (mg) | (mg) | (mg) | mg) | (mg) | (mg) | (mg) | (mg) | (mg) |
| 1 | PBS (MDX) | 1-1 | 25.9 | 25.6 | 27 | 25 | 27.6 | 29 | 21.2 | 23.5 | 24.3 | 27.8 | 24.3 | 25.6 |
| | | 1-2 | 28.2 | 24.5 | 26.5 | 26 | 28.2 | 24.4 | 30.2 | 23.1 | 22.6 | 23 | 27.7 | 27.5 |
| | | 1-3 | 28.9 | 27.7 | 24.4 | 26.1 | 27.7 | 29.3 | 20.3 | 24.4 | 30.9 | 26 | 23.4 | 25.6 |
| | | 1-4 | 26.7 | 28.1 | 27.5 | 28.8 | 26.3 | 26.6 | 23.3 | 27.4 | 27.8 | 29.8 | 28.1 | 26.9 |
| | | 1-5 | 26.5 | 23 | 23.3 | 25 | 28.2 | 25.6 | 24.6 | 20.7 | 24.8 | 28 | 24.6 | 23.5 |
| 2 | PMO (mdx) | 2-1 | 24.3 | 23.2 | 24.8 | 23.5 | 26.6 | 28.6 | 24.4 | 22.9 | 27.6 | 28.4 | 25.4 | 27.6 |
| | | 2-2 | 22.9 | 28.1 | 26.1 | 27.6 | 25.3 | 23.1 | 22.9 | 22.2 | 20.3 | 28.6 | 27.4 | 26 |
| | | 2-3 | 23.2 | 27.9 | 26 | 27.5 | 27.6 | 26 | 27.0 | 21.1 | 22.5 | 30.3 | 20.9 | 27.1 |
| | | 2-4 | 26 | 22.4 | 22 | 28.6 | 24.9 | 23.8 | 21.0 | 28.9 | 23.4 | 23.2 | 25 | 25.1 |
| | | 2-5 | 23.9 | 28.5 | 27.7 | 29.7 | 29.9 | 27 | 21.2 | 23.8 | 24.5 | 26.6 | 22.1 | 26.5 |
| 3 | PPMO (mdx) | 3-1 | 21.5 | 30.7 | 28.4 | 26.5 | 22.3 | 29.3 | 25.5 | 27.0 | 21.4 | 30.5 | 20.8 | 23.4 |
| | | 3-2 | 23.6 | 26.3 | 23.1 | 26.2 | 28.6 | 26.5 | 23.9 | 20.1 | 25.8 | 21.8 | 24.6 | 28.5 |
| | | 3-3 | 21.7 | 25.2 | 24.2 | 23.8 | 24.8 | 22.8 | 20.5 | 27.8 | 27.1 | 24.3 | 26.6 | 24.6 |
| | | 3-4 | 22.5 | 24.3 | 30.1 | 24.3 | 30.5 | 27.1 | 26.9 | 27.5 | 26.3 | 25.4 | 25.2 | 29.2 |
| | | 3-5 | 23.7 | 22.2 | 28.9 | 22.8 | 23.5 | 23.7 | 23.4 | 23.2 | 28.5 | 29.8 | 26.5 | 24.9 |
| 4 | CD71-PMO | 4-1 | 27.3 | 26.3 | x | 25.6 | 27.8 | 23.0 | 29.4 | 27.0 | 24.3 | 25.4 | 22.2 | x |
| | DAR 1.7 (mdx) | 4-2 | 25.0 | 23.4 | x | 26.8 | 29.7 | 28.2 | 25.2 | 29.0 | 26.8 | 28.4 | 27.8 | x |
| | | 4-3 | 23.9 | 28.0 | x | 27.7 | 30.7 | 22.8 | 25.5 | 25.4 | 29.9 | 29.5 | 28.7 | x |
| | | 4-4 | 26.9 | 29.7 | x | 23.8 | 28.0 | 23.8 | 24.6 | 26.7 | 22.4 | 30.6 | 24.3 | x |
| | | 4-5 | 27.2 | 29.2 | x | 27.1 | 23.0 | 21.6 | 27.6 | 27.8 | 25.6 | 29.1 | 23.6 | x |
| 5 | CD71-PMO | 5-1 | 25.7 | 27.9 | x | 30.8 | 28.3 | 27.0 | 26.6 | 25.5 | 26.5 | 30.1 | 23.7 | x |
| | DAR 1.7 (mdx) | 5-2 | 31.0 | 26.2 | x | 25.5 | 27.6 | 25.5 | 26.9 | 30.4 | 25.9 | 25.4 | 25.8 | x |
| | | 5-3 | 30.0 | 29.7 | x | 28.1 | 28.9 | 27.5 | 28.7 | 28.8 | 25.9 | 25.2 | 29.4 | x |
| | | 5-4 | 23.7 | 27.7 | x | 23.2 | 28.6 | 26.1 | 28.3 | 23.6 | 20.6 | 30.2 | 25.4 | x |
| | | 5-5 | | | x | | | | | | | | | x |
| 6 | CD71-PMO | 6-1 | 25.8 | 20.8 | 24.9 | 28.2 | 28.3 | 30.1 | 23.1 | 25.9 | 27.1 | 25.2 | 25.4 | 24.2 |
| | DAR 3.5 (mdx) | 6-2 | 23.6 | 22.3 | 30.0 | 26.3 | 27.4 | 27.1 | 26.3 | 27.0 | 29.5 | 27.1 | 23.6 | 25.8 |
| | | 6-3 | 29.8 | 23.1 | 28.6 | 27.2 | 26.9 | 24.2 | 23.6 | 29.1 | 29.1 | 21.4 | 24.4 | 28.9 |
| | | 6-4 | 26.1 | 22.1 | 28.3 | 27.6 | 27.3 | 28.4 | 25.0 | 27.9 | 27.9 | 30.6 | 28.3 | 28.2 |
| | | 6-5 | 22.2 | 29.3 | 27.5 | 28.2 | 28 | 21.1 | 24.8 | 30.3 | 27.8 | 27.8 | 23.8 | 29.8 |
| 7 | CD71-PMO | 7-1 | 22.2 | 26.3 | x | 20.7 | 26.5 | 24.9 | 24.9 | 23.3 | 24.5 | 27.4 | 25 | x |
| | DAR 3.5 (mdx) | 7-2 | 27.2 | 24.2 | x | 27.9 | 27.9 | 28.6 | 24.2 | 26.6 | 23.6 | 27.9 | 27.2 | x |
| | | 7-3 | 28.2 | 27.1 | x | 24.9 | 28.6 | 25 | 24.3 | 22.9 | 21.3 | 24.7 | 28.4 | x |
| | | 7-4 | 27.4 | 26.1 | x | 23.2 | 25.3 | 25.4 | 25.7 | 23.4 | 24.7 | 29.9 | 22 | x |
| | | 7-5 | 26.0 | 30.8 | x | 23.3 | 27 | 25.1 | 27.1 | 26.6 | 25.1 | 26 | 25.1 | x |
| 8 | CD71-PPMO | 8-1 | 23.4 | 24.7 | x | 23.6 | 26.9 | 23.1 | 23.6 | 25.9 | 25.1 | 25.7 | 27.9 | x |
| | DAR 1.7 (mdx) | 8-2 | 21.9 | 25.4 | x | 25.8 | 22.8 | 26.1 | 24.9 | 26.1 | 21.4 | 26.6 | 24.4 | x |
| | | 8-3 | 25.5 | 24.8 | x | 25.7 | 26.7 | 21.5 | 22.3 | 24 | 28.9 | 22.4 | 24.8 | x |
| | | 8-4 | 25.3 | 27.1 | x | 24.1 | 29.0 | 20.6 | 29.8 | 26.8 | 25.9 | 24.6 | 26 | x |
| | | 8-5 | | | x | | | | | | | | | x |
| 9 | CD71-PPMO | 9-1 | 21.1 | 25.0 | x | 29.2 | 29.2 | 26.7 | 22.8 | 23.7 | 22.0 | 22.9 | 23.5 | x |
| | DAR 1.7 (mdx) | 9-2 | 23.7 | 29.0 | x | 29.5 | 26.9 | 23.4 | 28.9 | 26.2 | 29.1 | 24.3 | 25.2 | x |
| | | 9-3 | 27.3 | 25.2 | x | 25.6 | 24.4 | 27.2 | 28.2 | 29.0 | 26.3 | 24.5 | 26.5 | x |
| | | 9-4 | 25.9 | 26.0 | x | 23.3 | 24.6 | 24.7 | 24.2 | 26.2 | 29.3 | 28.9 | 26.5 | x |
| | | 9-5 | 28.2 | 23.5 | x | 28.8 | 27.5 | 23.4 | 30.1 | 23.8 | 22.4 | 28.6 | 26.7 | x |
| 10 | CD71-PPMO | 10- | 26.4 | 25.7 | 24.3 | 23.0 | 26.5 | 27.5 | 23.2 | 28.4 | 27.2 | 25.3 | 27.2 | 29.2 |
| | DAR 3.5 (mdx) | 1 | | | | | | | | | | | | |
| | | 10- | 20.2 | 29.0 | 23.1 | 22.2 | 26.7 | 25.6 | 23.9 | 26.8 | 22.3 | 28.5 | 20.3 | 26.0 |
| | | 2 | | | | | | | | | | | | |
| | | 10- | 27.5 | 28.8 | 27.8 | 24.1 | 26.5 | 26.3 | 29.7 | 26.6 | 24.5 | 28.5 | 23.6 | 24.1 |
| | | 3 | | | | | | | | | | | | |
| | | 10- | 24.4 | 21.4 | 22.0 | 21.4 | 27.3 | 29.9 | 22.9 | 29.4 | 27.3 | 26.1 | 22.1 | 25.0 |
| | | 4 | | | | | | | | | | | | |
| | | 10- | 27.4 | 24.4 | 24.1 | 26.5 | 27.5 | 25.6 | 27.9 | 27.6 | 25.8 | 21.2 | 29.8 | 26.9 |
| | | 5 | | | | | | | | | | | | |
| 11 | CD71-PPMO | 11- | 25.1 | 26.1 | x | 22.2 | 25.9 | 27.9 | 21.8 | 24.4 | 21.9 | 26.2 | 24.7 | x |
| | DAR 3.5 (mdx) | 1 | | | | | | | | | | | | |
| | | 11- | 26.1 | 30.3 | x | 26.8 | 26.6 | 27.3 | 23.3 | 23.9 | 29.6 | 23.1 | 28.5 | x |
| | | 2 | | | | | | | | | | | | |
| | | 11- | 24.0 | 29.4 | x | 24.4 | 26.9 | 22.6 | 21.2 | 26.2 | 21.9 | 28.1 | 23 | x |
| | | 3 | | | | | | | | | | | | |
| | | 11- | 29.8 | 29.6 | x | 23.8 | 23.9 | 21.3 | 26.2 | 23.4 | 30.5 | 28.4 | 27.2 | x |
| | | 4 | | | | | | | | | | | | |
| | | 11- | 28.0 | 25.2 | x | 23.2 | 22.4 | 25.8 | 30.3 | 26.5 | 25.4 | 20.2 | 23.3 | x |
| | | 5 | | | | | | | | | | | | |
| 12 | Isotype mAb- | 12- | 20.0 | 20.6 | 28.0 | 22.9 | 26.0 | 25 | 22.1 | 27.4 | 27.3 | 24.3 | 22.2 | 25.4 |
| | PPMO DAR 3.5 | 1 | | | | | | | | | | | | |
| | (mdx) | 12- | 28.9 | 28.4 | 23.8 | 23.8 | 26.5 | 26.5 | 29.7 | 28.4 | 27.5 | 22.2 | 25.6 | 29.5 |
| | | 2 | | | | | | | | | | | | |
| | | 12- | 24.5 | 23.5 | 30.8 | 27.4 | 27.7 | 30.5 | 28.3 | 25.8 | 25.2 | 29 | 20.8 | 30.1 |
| | | 3 | | | | | | | | | | | | |
| 13 | PBS (C57/B16) | 13- | 25.3 | 29.8 | 25.5 | 22.3 | 24.3 | 20.5 | 20.7 | 22.2 | 21.1 | 26.5 | 23.8 | 26.0 |
| | | 1 | | | | | | | | | | | | |
| | | 13- | 28.2 | 29.9 | 24.6 | 27.8 | 20.1 | 20.7 | 27.2 | 20.2 | 28 | 30.2 | 20.3 | 23.9 |
| | | 2 | | | | | | | | | | | | |
| | | 13- | 26.2 | 26.6 | 23.4 | 24.1 | 20.4 | 24.1 | 20.1 | 23 | 24.6 | 22.4 | 21.9 | 26.9 |
| | | 3 | | | | | | | | | | | | |
| | | 13- | 24.5 | 28.2 | 24.1 | 27.1 | 20.9 | 24.1 | 26.4 | 25.8 | 30.2 | 25.7 | 27.4 | 28.6 |
| | | 4 | | | | | | | | | | | | |
| | | 13- | 26.0 | 27.6 | 25.7 | 22.0 | 22.3 | 22.9 | 24.6 | 21.5 | 20.9 | 30.5 | 26.7 | 24.4 |
| | | 5 | | | | | | | | | | | | |

FIG. 42 and Tables 31-35 show mouse exon 23 PMO/PPMO standard curves in various tissue homogenates, reflecting the same percentage of tissue homogenate in diluted samples. These standard curves are used to interpolate the concentration of PMO or PPMO in a given sample.

TABLE 31
TA (10%) standard 60 minutes AP incubation statistics

| Best-fit values | PMO (10% TA) | P-PMO (10% TA) |
|---|---|---|
| Top | 38570 | 39371 |
| Bottom | 2084 | 2015 |
| LogIC50 | 2.529 | 2.507 |
| Hill Slope | 1.606 | 1.587 |
| IC50 | 337.8 | 321.6 |
| Span | 36486 | 37356 |
| R squared | 0.9985 | 0.9943 |

TABLE 32
Gastroc (10%) standard 60 minutes AP incubation statistics

| Best-fit values | PMO (10% Gastroc) | P-PMO (10% Gastroc) |
|---|---|---|
| Top | 37648 | 38094 |
| Bottom | 1554 | 1818 |
| LogIC50 | 2.612 | 2.636 |
| Hill Slope | 1.562 | 1.715 |
| IC50 | 409.2 | 432.3 |
| Span | 36094 | 36276 |
| R squared | 0.9973 | 0.9968 |

TABLE 33
Diaphragm (10%) standard 60 minutes AP incubation statistics

| Best-fit values | PMO (10% Diaphragm) | P-PMO (10% Diaphragm) |
|---|---|---|
| Top | 36585 | 36700 |
| Bottom | 1394 | 1411 |
| LogIC50 | 2.79 | 2.758 |
| Hill Slope | 1.692 | 1.707 |
| IC50 | 617.2 | 573.3 |
| Span | 35191 | 35290 |
| R squared | 0.9968 | 0.9902 |

TABLE 34
Heart (10%) standard 60 minutes AP incubation statistics

| Best-fit values | PMO (10% TA) | P-PMO (10% TA) |
|---|---|---|
| Top | 36375 | 36371 |
| Bottom | 875.2 | 1840 |
| LogIC50 | 2.717 | 2.724 |
| Hill Slope | 1.5 | 1.792 |
| IC50 | 521.7 | 529.2 |
| Span | 35500 | 34530 |
| R squared | 0.9959 | 0.9912 |

TABLE 35
Liver (10%) standard 60 minutes AP incubation statistics

| Best-fit values | PMO (10% Liver) | P-PMO (10% Liver) |
|---|---|---|
| Top | 20467 | 26492 |
| Bottom | 1039 | 1341 |
| LogIC50 | 3.056 | 2.968 |
| Hill Slope | 2.116 | 2.226 |
| IC50 | 1137 | 928.1 |
| Span | 19428 | 25150 |
| R squared | 0.9953 | 0.9849 |

### Example 28. In Vivo Exon 23 Skipping Activity and Exon 23 PMO Concentrations in Muscles of Mdx Mice Administered a Dose of PPMO-AOCs Having the Orientation 1 or Orientation 2

Table 36 lists the names, type of linkers, and orientation for each of the tested PPMO-AOCs that is administered to mdx mice for the in vivo exon 23 skipping activity and exon 23 PMO concentrations studies.

TABLE 36
Lists the names, type of linkers, and orientation
for each of the tested PPMO-AOCs

| | | Orienta- |
|---|---|---|
| PPMO-AOC | Linker Type | tion |
| anti-mCD71 Ab-PMO23-(RXR)4XB | Non-cleavable | 1 |
| (DAR 1.7) | | |
| anti-mCD71 Ab-PMO23-(RXR)4XB | Non-cleavable | 1 |
| (DAR 3.5) | | |
| Isotype Ab-PMO23-(RXR)4XB (DAR 3.5) | Non-cleavable | 1 |
| anti-mCD71 Ab-MC-RXR)4XB-PMO23 | Non-cleavable | 2 |
| anti-mCD71 Ab-MC-VC-(RXR)4XB- | Cleavable | 2 |
| PMO23 | | |
| anti-mCD71 Ab-MC-Pip6a-PMO23 | Non-cleavable | 2 |
| anti-mCD71 Ab-MC-VC-Pip6a-PMO23 | Cleavable | 2 |

### Synthesis, Purification, and Characterization of Cell Penetrating Peptide-Modified Antisense Phosphorodiamidate Morpholino Oligomers (PPMOs)

### Synthesis of MC-RXR4XB-PM023

### Step 1: Synthesis and Purification of Fmoc-(RXR)₄XB-PM023 PPMO

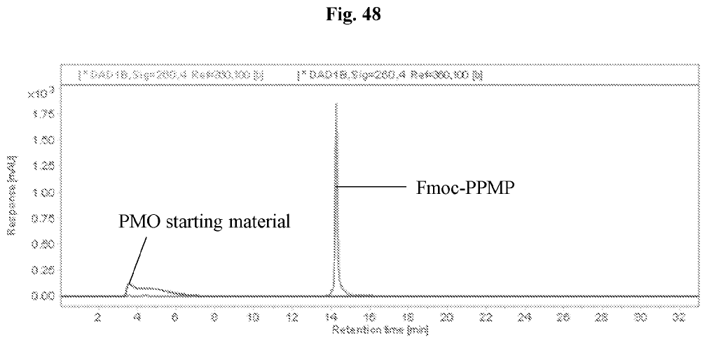

Lyophilized 3′-NH2 functionalized PMO for skipping mouse exon 23 (PM023 PMO, 15.3 mg, sequence: GGCCAAACCTCGGCTTACCTGAAAT (SEQ ID NO:28)) was dissolved in anhydrous dimethyl sulfoxide (DMSO, 306 μL). In a separate vial, Fmoc-(RXR)4XB peptide (“(RXR)4XB” disclosed as SEQ ID NO: 1065) (4 molar equivalents, 15.3 mg) and 1-[Bis(dimethylamino)methylene]-1H-1,2,3-triazolo[4,5-b]pyridinium 3-oxide hexafluorophosphate (HATU, 6 molar equivalents, 2.7 mg) were dissolved in a mixture of anhydrous DMSO (232 μL) and N,N-Diisopropylethylamine (DIPEA, 10 molar equivalents, 3.14 μL) and let sit for 5 minutes. The PMO solution and the peptide solution were combined, and the reaction was left for 1 hour at room temperature. The reaction mixture was diluted to 15 mL with nanopure water. The reaction was concentrated in a 15 mL 3,000 MWCO Amicon spin filter (4,000 xg for 30 minutes), diluted to 15 mL with nanopure water, and repeated for a total of 3 washes. The final retentate was not diluted with water. The reaction was analyzed using SCX Method 6 (FIG. 48). The reaction mixture was purified by SCX purification following SCX method 1. The pooled fractions containing product were concentrated in 15 mL 3,000 MWCO Amicon spin filters (4,000×g for 30 min), diluted to 15 mL with nanopure water, and repeated for a total of 5 washes. The final retentate was not diluted with water. The product was then filtered through a sterile 0.22 um syringe filter to grant the product as a clear and colorless solution which was quantified by UV VIS (12.5 mg, 1180 nmol, 65% yield).

### Step 2: Deprotection of Fmoc-(RXR)₄XB-PM023 PPMO

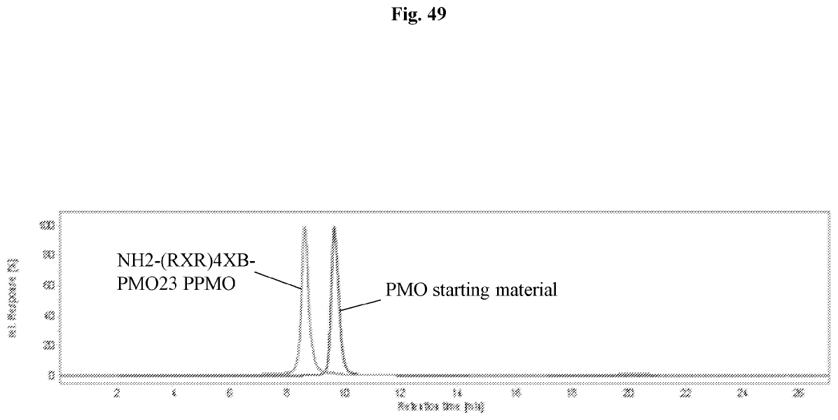

To a solution of fmoc-(RXR)4XB-PM023 PPMO dissolved in water (53 μM, 13.0 mL) was added diethyl amine (1.5 ml) and the reaction was allowed to proceed at room temperature for 90 minutes. The reaction mixture was concentrated in 15 mL 3,000 MWCO Amicon spin filters (4,000×g for 30 min), diluted to 15 mL with nanopure water, and repeated for a total of 5 washes. The final retentate containing the product was not diluted with water. The product was analyzed using reversed-phase HPLC (RP-HPLC) method 1 (FIG. 49). The sample was frozen and lyophilized (4.8 mg, 71% yield).

### Step 3: Synthesis and Purification of MC-(RXR)₄XB-PM023 PPMO

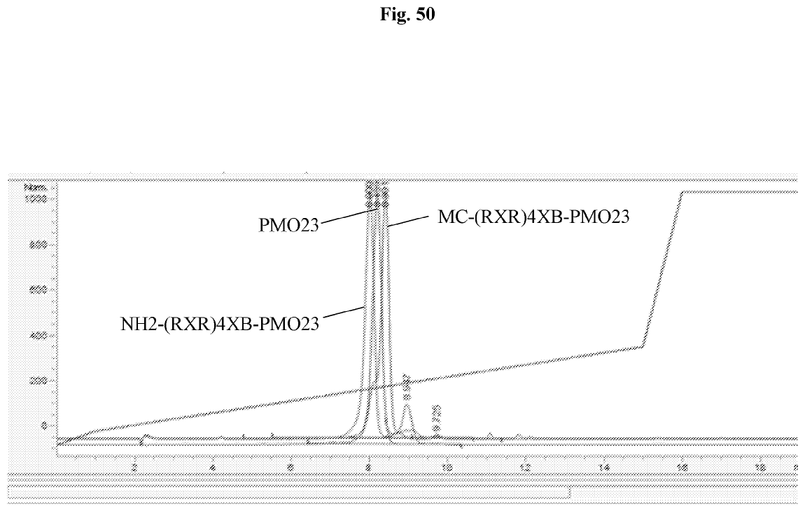

Lyophilized NH2-(RXR)4XB-PM023 PPMO for skipping mouse exon 23 (PM023 PMO, 2.7 mg, sequence: GGCCAAACCTCGGCTTACCTGAAAT (SEQ ID NO:28)) was dissolved in anhydrous dimethyl sulfoxide (DMSO, 251.6 μL). In a separate vial, maleimidocaprioic acid (MC, 4 molar equivalents, 0.22 mg) and 1-[Bis(dimethylamino)methylene]-1H-1,2,3-triazolo[4,5-b]pyridinium 3-oxide hexafluorophosphate (HATU, 4 molar equivalents, 0.40 mg) were dissolved in a mixture of anhydrous DMSO (24.6 μL) and N,N-Diisopropylethylamine (DIPEA, 5 molar equivalents, 23 μL of a solution of DIPEA diluted 100-fold into DMSO) and let sit for 5 minutes. The PPMO solution and the MC/HATU activation solution were combined, and the reaction was left for 1 h at room temperature. The reaction mixture was diluted to 5 mL with nanopure water. The diluted reaction mixture was concentrated in 15 mL 3,000 MWCO Amicon spin filters (14,000×g for 10 min), diluted to 5 mL with nanopure water, and repeated for a total of 3 washes. The final retentate was not diluted with water and was recovered as a clear and colorless solution (yield 52%). The reaction was analyzed using reversed-phase HPLC (RP-HPLC) method 1 (FIG. 50).

### Synthesis of MC-VC-(RXR)₄XB-PMO23

### Step 1: Synthesis and Purification of Fmoc-VC-(RXR)₄XB-PM023 PPMO

Lyophilized 3′-NH2 functionalized PMO for skipping mouse exon 23 (PM023 PMO, 4.3 mg, sequence: GGCCAAACCTCGGCTTACCTGAAAT (SEQ ID NO:28)) was dissolved in anhydrous dimethyl sulfoxide (DMSO, 267 μL). In a separate vial, Fmoc-VC-(RXR)4XB peptide (“VC-(RXR)4XB” disclosed as SEQ ID NO: 1071) (4 molar equivalents, 22.5 mg) and 1-[Bis(dimethylamino)methylene]-1H-1,2,3-triazolo[4,5-b]pyridinium 3-oxide hexafluorophosphate (HATU, 4 molar equivalents, 3.6 mg) were dissolved in a mixture of anhydrous DMSO (316 μL) and N,N-Diisopropylethylamine (DIPEA, 10 molar equivalents, 4.11 uL) and let sit for 5 minutes. The PMO solution and the peptide solution were combined, and the reaction was left for 1 h at room temperature. The reaction mixture was diluted to 15 mL with nanopure water. The reaction was concentrated in a 15 mL 3,000 MWCO Amicon spin filter (4,000 xg for 30 min), diluted to 15 mL with nanopure water, and repeated for a total of 3 washes. The final retentate was not diluted with water. The reaction was analyzed using SCX Method 6. The reaction mixture was purified by SCX purification following SCX method 1. The pooled fractions containing product were concentrated in 15 mL 3,000 MWCO Amicon spin filters (4,000×g for 30 min), diluted to 15 mL with nanopure water, and repeated for a total of 5 washes. The final retentate was not diluted with water. The product was then filtered through a sterile 0.22 um syringe filter to grant the product as a clear and colorless solution which was quantified by UV VIS (3.3 mg, 304 nmol, 60% yield). Note: VC=Valine-Citrulline

### Step 2: Deprotection of Fmoc-VC-(RXR)₄XB-PM023 PPMO

To a solution of Fmoc-VC-(RXR)4XB-PM023 PPMO dissolved in water (53 μM, 17.5 mL) was added diethyl amine (2.0 ml) and the reaction was allowed to proceed at room temperature for 90 minutes. The reaction mixture was concentrated in 15 mL 3,000 MWCO Amicon spin filters (4,000×g for 30 min), diluted to 15 mL with nanopure water, and repeated for a total of 5 washes. The final retentate containing the product was not diluted with water. The product was analyzed using reversed-phase HPLC (RP-HPLC) method 1. The sample was frozen and lyophilized (6.3 mg, 64% yield). Note: VC=Valine-Citrulline

### Step 3: Synthesis and Purification of MC-VC-(RXR)₄XB-PM023 PPMO

Lyophilized NH2-(RXR)4XB-PM023 PPMO for skipping mouse exon 23 (PM023 PMO, 5.1 mg, sequence: GGCCAAACCTCGGCTTACCTGAAAT (SEQ ID NO:28)) was dissolved in anhydrous dimethyl sulfoxide (DMSO, 1050 μL). In a separate vial, maleimidocaprioic acid (MC, 4 molar equivalents, 0.41 mg) and 1-[Bis(dimethylamino)methylene]-1H-1,2,3-triazolo[4,5-b]pyridinium 3-oxide hexafluorophosphate (HATU, 4 molar equivalents, 0.73 mg) were dissolved in a mixture of anhydrous DMSO (45.6 μL) and N,N-Diisopropylethylamine (DIPEA, 5 molar equivalents, 43 μL of a solution of DIPEA diluted 100-fold into DMSO) and let sit for 5 minutes. The PPMO solution and the MC/HATU activation solution were combined, and the reaction was left for 1 h at room temperature. The reaction mixture was diluted to 5 mL with nanopure water. The diluted reaction mixture was concentrated in 15 mL 3,000 MWCO Amicon spin filters (14,000×g for 10 min), diluted to 5 mL with nanopure water, and repeated for a total of 3 washes. The final retentate was not diluted with water and was recovered as a clear and colorless liquid (67% yield). The reaction was analyzed using reversed-phase HPLC (RP-HPLC) method 1. Note: VC=Valine-Citrulline

### Synthesis of MC-Pip6a-PM023

### Step 1: Synthesis and Purification of Fmoc-Pip6a-PM023 PPMO

Lyophilized 3′-NH2 functionalized PMO for skipping mouse exon 23 (PM023 PMO, 40 mg, sequence: GGCCAAACCTCGGCTTACCTGAAAT (SEQ ID NO:28)) was dissolved in anhydrous dimethyl sulfoxide (DMSO, 718 μL). In a separate vial, Fmoc-VC-(RXR)4XB peptide (“VC-(RXR)4XB” disclosed as SEQ ID NO: 1071) (3.5 molar equivalents, 52 mg) and 1-[Bis(dimethylamino)methylene]-1H-1,2,3-triazolo[4,5-b]pyridinium 3-oxide hexafluorophosphate (HATU, 3.5 molar equivalents, 6.3 mg) were dissolved in a mixture of anhydrous DMSO (689 μL) and N,N-Diisopropylethylamine (DIPEA, 10 molar equivalents, 8.21 uL) and let sit for 5 minutes. The PMO solution and the peptide solution were combined, and the reaction was left for 1 h at room temperature. The reaction mixture was diluted to 15 mL with nanopure water. The reaction was concentrated in a 15 mL 3,000 MWCO Amicon spin filter (4,000 xg for 30 min), diluted to 15 mL with nanopure water, and repeated for a total of 3 washes. The final retentate was not diluted with water. The reaction was analyzed using SCX Method 6. The reaction mixture was purified by SCX purification following SCX method 1. The pooled fractions containing product were concentrated in 15 mL 3,000 MWCO Amicon spin filters (4,000×g for 30 min), diluted to 15 mL with nanopure water, and repeated for a total of 5 washes. The final retentate was not diluted with water. The product was then filtered through a sterile 0.22 um syringe filter to grant the product as a clear and colorless solution which was quantified by UV VIS (11.4 mg, 983 nmol, 21% yield).

### Step 2: Deprotection of Fmoc-Pip6a-PM023 PPMO

To a solution of Fmoc-Pip6a-PMO23 PPMO (11.4 mg) was dissolved in 20% diethylamine/80% DMSO (1 mL) and the reaction was allowed to proceed at room temperature for 60 minutes. The reaction mixture was concentrated in 15 mL 3,000 MWCO Amicon spin filters (4,000×g for 30 min), diluted to 15 mL with nanopure water, and repeated for a total of 5 washes. The final retentate containing the product was not diluted with water. The product was analyzed using reversed-phase HPLC (RP-HPLC) method 1. The sample was frozen and lyophilized (1.75 mg, 16% yield).

### Step 3: Synthesis and Purification of MC-Pip6a-PM023 PPMO

Lyophilized NH2-(RXR)4XB-PM023 PPMO for skipping mouse exon 23 (PM023 PMO, 2.7 mg, sequence: GGCCAAACCTCGGCTTACCTGAAAT (SEQ ID NO:28)) was dissolved in anhydrous dimethyl sulfoxide (DMSO, 251.6 μL). In a separate vial, maleimidocaprioic acid (MC, 4 molar equivalents, 0.22 mg) and 1-[Bis(dimethylamino)methylene]-1H-1,2,3-triazolo[4,5-b]pyridinium 3-oxide hexafluorophosphate (HATU, 4 molar equivalents, 0.40 mg) were dissolved in a mixture of anhydrous DMSO (24.6 uL) and N,N-Diisopropylethylamine (DIPEA, 5 molar equivalents, 23 μL of a solution of DIPEA diluted 100-fold into DMSO) and let sit for 5 minutes. The PPMO solution and the MC/HATU activation solution were combined, and the reaction was left for 1 hour at room temperature. The reaction mixture was diluted to 5 mL with nanopure water. The diluted reaction mixture was concentrated in 15 mL 3,000 MWCO Amicon spin filters (14,000×g for 10 min), diluted to 5 mL with nanopure water, and repeated for a total of 3 washes. The final retentate was not diluted with water. The reaction was analyzed using reversed-phase HPLC (RP-HPLC) method 1.

### Synthesis of MC-VC-Pip6a-PM023

### Step 1: Synthesis and Purification of Fmoc-VC-Pip6a-PM023 PPMO

Lyophilized 3′-NH2 functionalized PMO for skipping mouse exon 23 (PM023 PMO, 40 mg, sequence: GGCCAAACCTCGGCTTACCTGAAAT (SEQ ID NO:28)) was dissolved in anhydrous dimethyl sulfoxide (DMSO, 718 μL). In a separate vial, Fmoc-VC-Pip6a peptide (3.5 molar equivalents, 56 mg) and 1-[Bis(dimethylamino)methylene]-1H-1,2,3-triazolo[4,5-b]pyridinium 3-oxide hexafluorophosphate (HATU, 3.5 molar equivalents, 6.3 mg) were dissolved in a mixture of anhydrous DMSO (63 μL) and N,N-Diisopropylethylamine (DIPEA, 10 molar equivalents, 8.21 uL) and let sit for 5 minutes. The PMO solution and the peptide solution were combined, and the reaction was left for 1 h at room temperature. The reaction mixture was diluted to 15 mL with nanopure water. The reaction was concentrated in a 15 mL 3,000 MWCO Amicon spin filter (4,000 xg for 30 min), diluted to 15 mL with nanopure water, and repeated for a total of 3 washes. The final retentate was not diluted with water. The reaction was analyzed using SCX Method 6. The reaction mixture was purified by SCX purification following SCX method 1. The pooled fractions containing product were concentrated in 15 mL 3,000 MWCO Amicon spin filters (4,000×g for 30 min), diluted to 15 mL with nanopure water, and repeated for a total of 5 washes. The final retentate was not diluted with water. The product was then filtered through a sterile 0.22 μm syringe filter to grant the product as a clear and colorless solution which was quantified by UV VIS (14.6 mg, 1231 nmol, 26% yield). Note: VC=Valine-Citrulline

### Step 2: Deprotection of Fmoc-VC-Pip6a-PM023 PPMO

To a solution of Fmoc-VC-Pip6a-PM023 PPMO (14.6 mg) was dissolved in 20% diethylamine/80% DMSO (1 mL) and the reaction was allowed to proceed at room temperature for 60 minutes. The reaction mixture was concentrated in 15 mL 3,000 MWCO Amicon spin filters (4,000×g for 30 min), diluted to 15 mL with nanopure water, and repeated for a total of 5 washes. The final retentate containing the product was not diluted with water. The product was analyzed using reversed-phase HPLC (RP-HPLC) method 1. The sample was frozen and lyophilized (2.68 mg, 19% yield). Note: VC=Valine-Citrulline

### Step 3: Synthesis and Purification of MC-VC-Pip6a-PM023 PPMO

Lyophilized NH2-VC-Pip6a-PM023 PPMO for skipping mouse exon 23 (PM023 PMO, 2.7 mg, sequence: GGCCAAACCTCGGCTTACCTGAAAT (SEQ ID NO:28)) was dissolved in anhydrous dimethyl sulfoxide (DMSO, 251.6 uL). In a separate vial, maleimidocaprioic acid (MC, 4 molar equivalents, 0.19 mg) and 1-[Bis(dimethylamino)methylene]-1H-1,2,3-triazolo[4,5-b]pyridinium 3-oxide hexafluorophosphate (HATU, 4 molar equivalents, 0.35 mg) were dissolved in a mixture of anhydrous DMSO (22 μL) and N,N-Diisopropylethylamine (DIPEA, 5 molar equivalents, 20 μL of a solution of DIPEA diluted 100-fold into DMSO) and let sit for 5 minutes. The PPMO solution and the MC/HATU activation solution were combined, and the reaction was left for 1 hour at room temperature. The reaction mixture was diluted to 5 mL with nanopure water. The diluted reaction mixture was concentrated in 15 mL 3,000 MWCO Amicon spin filters (14,000×g for 10 min), diluted to 5 mL with nanopure water, and repeated for a total of 3 washes. The final retentate was not diluted with water and was recovered as a clear and colorless liquid (yield 76%). The reaction was analyzed using reversed-phase HPLC (RP-HPLC) method 1. Note: VC=Valine-Citrulline

### Synthesis, Purification, and Characterization of Cell Penetrating Peptide-Modified Antisense Phosphorodiamidate Morpholino Oligomers Conjugated to a CD71 Antibody (PPMO-AOC) in the Antibody-Peptide-PMO Configuration

### Anti-Transferrin Receptor Antibody

Anti-mouse transferrin receptor antibody or rat anti-mouse CD71 IgG2a (anti-mCD71 Ab) that was used was a rat IgG2a subclass monoclonal antibody that binds mouse CD71 or mouse transferrin receptor 1 (mTfR1). The antibody was produced by BioXcell and it is commercially available (Catalog #BE0175).

### Anti-mCD71 Ab-MC-(RXR)₄XB-PM023

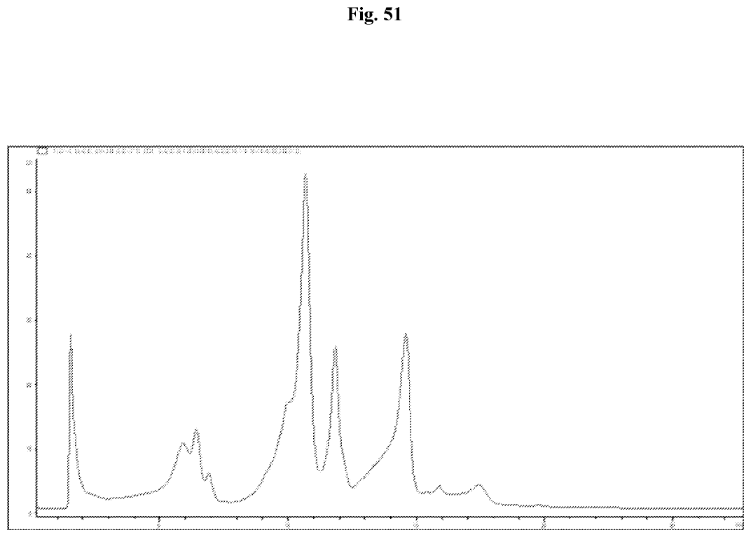

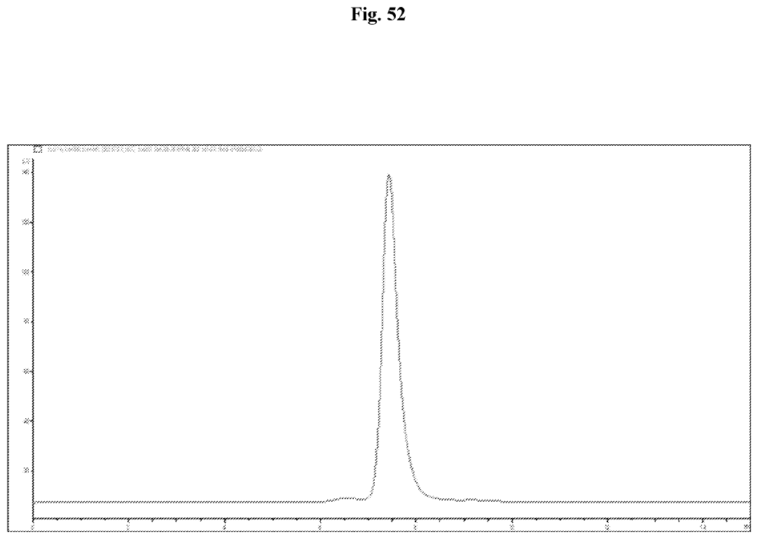

To the mCD71 Antibody (81 mg, 9.205 ml) in PBS buffer (137 mM NaCl, 2.7 mM KCl, 10 mM Na2HPO4, and 1.8 mM KH2PO4), pH 7.4) was added ethylenediaminetetraacetic acid (37 μl of a 0.5 M solution), and tris(2-carboxyethyl)phosphine (TCEP, 8 eq, 247.7 μL of a 5 mg/ml solution in water) and incubated at 37° C. for 2 hours. To an aliquot of the reduced antibody (9.2 mg. 1.08 mL) at room temperature was added MC-(RXR)4XB-PM023 (2 eq, 186.7 nmol) slowly by adding 0.5 eq PMO every 5 minutes. The reaction was allowed to proceed for 30 minutes before being cooled to 4° C. overnight. N-Ethylmaleimide (10 equivalents) of was added to the mixture at room temperature for 30 minutes to quench unreacted cysteines. The reaction mixture was diluted with PBS and was concentrated and buffer exchanged with PBS (pH 7.4) using Amicon Ultra-15 centrifugal filter with a MWCO of 50 kDa units. The product was then filtered through a sterile 0.22 μm syringe filter to grant the product as a clear and colorless solution and analyzed by strong cation exchange (SCX) chromatography method 7 (FIG. 51) and Size exclusion chromatography method 1 (FIG. 52). The product was quantified by BCA to measure antibody concentration (0.49 ml, 12.8 mg/mL, 78% yield).

### Anti-mCD71 Ab-MC-VC-(RXR)₄XB-PM023

To the mCD71 Antibody (81 mg, 9.205 ml) in PBS buffer (137 mM NaCl, 2.7 mM KCl, 10 mM Na2HPO4, and 1.8 mM KH2PO4), pH 7.4) was added ethylenediaminetetraacetic acid (37 μl of a 0.5 M solution), and tris(2-carboxyethyl)phosphine (TCEP, 8 eq, 247.7 uL of a 5 mg/ml solution in water) and incubated at 37° C. for 2 hours. To an aliquot of the reduced antibody (24 mg, 2.81 mL) at room temperature was added MC-VC-(RXR)4XB-PM023 (2 eq, 542 nmol) slowly by adding 0.5 eq PMO every 5 minutes. The reaction was allowed to proceed for 30 minutes before being cooled to 4° C. overnight. N-Ethylmaleimide (10 equivalents) of was added to the mixture at room temperature for 30 minutes to quench unreacted cysteines. The reaction mixture was diluted with PBS and was concentrated and buffer exchanged with PBS (pH 7.4) using Amicon Ultra-15 centrifugal filter with a MWCO of 50 kDa units. The product was then filtered through a sterile 0.22 μm syringe filter to grant the product as a clear and colorless solution and analyzed by strong cation exchange (SCX) chromatography method 7 and size exclusion chromatography method 1. The product was quantified by BCA to measure antibody concentration (1.46 ml, 21.6 mg/mL, 72% yield). Note: VC=Valine-Citrulline

### Anti-mCD71 Ab-MC-Pip6a-PM023

To the mCD71 Antibody (81 mg, 9.205 ml) in PBS buffer (137 mM NaCl, 2.7 mM KCl, 10 mM Na2HPO4, and 1.8 mM KH2PO4), pH 7.4) was added ethylenediaminetetraacetic acid (37 μl of a 0.5 M solution), and tris(2-carboxyethyl)phosphine (TCEP, 8 eq, 247.7 μL of a 5 mg/ml solution in water) and incubated at 37° C. for 2 hours. To an aliquot of the reduced antibody (6 mg, 0.70 mL) at room temperature was added MC-Pip6a-PM023 (1.5 eq, 113 nmol) slowly by adding 0.5 eq PMO every 5 minutes. The reaction was allowed to proceed for 30 minutes before being cooled to 4 C overnight. N-Ethylmaleimide (10 equivalents) of was added to the mixture at room temperature for 30 minutes to quench unreacted cysteines. The reaction mixture was diluted with PBS and was concentrated and buffer exchanged with PBS (pH 7.4) using Amicon Ultra-15 centrifugal filter with a MWCO of 50 kDa units. The product was then filtered through a sterile 0.22 μm syringe filter to grant the product as a clear and colorless solution and analyzed by strong cation exchange (SCX) chromatography method 7 and size exclusion chromatography method 1. The product was quantified by BCA to measure antibody concentration (0.40 ml, 11.8 mg/mL, 79% yield).

### Anti-mCD71 Ab-MC-VC-Pip6a-PM023

To the mCD71 Antibody (81 mg, 9.205 ml) in PBS buffer (137 mM NaCl, 2.7 mM KCl, 10 mM Na2HPO4, and 1.8 mM KH2PO4), pH 7.4) was added ethylenediaminetetraacetic acid (37 μl of a 0.5 M solution), and tris(2-carboxyethyl)phosphine (TCEP, 8 eq, 247.7 μL of a 5 mg/ml solution in water) and incubated at 37° C. for 2 hours. To an aliquot of the reduced antibody (9.5 mg, 1.11 mL) at room temperature was added MC-VC-Pip6a-PM023 (1.5 eq, 145 nmol) slowly by adding 0.5 eq PMO every 5 minutes. The reaction was allowed to proceed for 30 minutes before being cooled to 4° C. overnight. N-Ethylmaleimide (10 equivalents) of was added to the mixture at room temperature for 30 minutes to quench unreacted cysteines. The reaction mixture was diluted with PBS and was concentrated and buffer exchanged with PBS (pH 7.4) using Amicon Ultra-15 centrifugal filter with a MWCO of 50 kDa units. The product was then filtered through a sterile 0.22 μm syringe filter to grant the product as a clear and colorless solution and analyzed by strong cation exchange (SCX) chromatography method 7 and size exclusion chromatography method 1. The product was quantified by BCA to measure antibody concentration (0.52 ml, 14.3 mg/mL, 78% yield). Note: VC=Valine-Citrulline

### Analytical and Purification Methods

### Reversed-Phase HPLC (RP-HPLC) Method 1

1. Column: Phenomenex Aeris 3.6 μm WIDEPORE XB-C18 250×4.6 mm
2. Column Temperature: 80° C.
3. Solvent A: 0.1% TFA in water
4. Solvent B: 0.1% TFA in acetonitrile
5. Detectors: UV absorbance, 220, 260 and 280 nm
6. Flow Rate: 1 ml/min
7. Gradient:

| | Time | % A | % B |
|---|---|---|---|
| a. | 0.0 | 90 | 10 |
| b. | 1.0 | 85 | 15 |
| c. | 15.0 | 55 | 45 |
| d. | 16.0 | 0 | 100 |
| e. | 21.0 | 0 | 100 |
| f. | 21.5 | 90 | 10 |
| i. | 27.0 | 90 | 10 |

### Strong Cation Exchange Chromatography (SCX) Method 1

1. Column: GE HiPrep SP HP 16/10 20 mL CV
2. Column Temperature: 25° C.
3. Mobile phase A: 20 mM PB pH 7, 25% CAN
4. Mobile phase B: 20 mM PB pH 7, 1.5 M Guanidinium HCl, 25% acetonitrile
5. Flow rate: 3 ml/min
6. Gradient:

| | % B | Column Volume (CV) |
|---|---|---|
| a. | 10 | Load |
| b. | 50 | 0.25 |
| c. | 65 | 2.5 |
| d. | 100 | 0.25 |
| e. | 100 | 1 |

### Strong Cation Exchange Chromatography (SCX) Method 2

1. Column: GE 5 ml SP HP column
2. Column Temperature: 25° C.
3. Mobile phase A: 20 mm NaH2PO4 pH7, 25% ACN
4. Mobile phase B: 20 mm NaH2PO4, 1.5M guanidine HCl, pH7, 25% ACN
5. Detectors: UV absorbance, 220, 260 and 280 nm
6. Flow rate: 0.6 ml/min
7. Gradient:

| | % B | CV |
|---|---|---|
| a. | 50 | 0.25 |
| b. | 65 | 2.5 |
| c. | 100 | 0.25 |
| d. | 100 | 1 |
| e. | 100 | 1 |

### Strong Cation Exchange Chromatography (SCX) Method 3

1. Column: GE HiPrep SP HP 16/10 20 mL CV
2. Column Temperature: 25° C.
3. Mobile phase A: 1×PBS, pH 7.5, 10% ethanol
4. Mobile phase B: 1×PBS, pH 7.5, 10% ethanol, 1.5 M sodium chloride
5. Flow rate: 3 ml/min
6. Gradient:

| a. | % B | CV |
|---|---|---|
| b. | 10 | 0.5 |
| c. | 100 | 2 |
| d. | 100 | 2 |

### Strong Cation Exchange (SCX) Method 4

1. Column: Thermo Scientific, MabPacSCX-10 column 4×250 mm
2. Solvent A: 20 mM sodium phosphate pH 7, 25% Acetonitrile; Solvent B: 20 mM Sodium phosphate, 1.5M Guanidine HCl pH 7, 25% acetonitrile
3. Flow Rate: 0.65 ml/min
4. Gradient:

| | Time | % A | % B |
|---|---|---|---|
| a. | 0.00 | 100 | 0 |
| b. | 2.00 | 100 | 0 |
| c. | 32.00 | 0 | 100 |
| d. | 32.50 | 0 | 100 |
| e. | 33.00 | 100 | 0 |
| f. | 39.00 | 100 | 0 |

### Strong Cation Exchange (SCX) Method 5

1. Column: GE SP HP Column 4.7 ml
2. Solvent A: 20 mM sodium phosphate, 150 mM sodium chloride pH 7.4, 10% ethanol; Solvent B: 20 mM sodium phosphate, 1.5M sodium chloride pH 7.4, 10% ethanol
3. Flow Rate: 0.6 ml/min
4. Gradient:

| | % A | % B | CV |
|---|---|---|---|
| a. | 100 | 0 | 2 |
| b. | 0 | 50 | 8 |
| c. | 0 | 100 | 0.1 |

### Strong Cation Exchange (SCX) Method 6

1. Column: Thermo Scientific, ProPac SCX-10 HPLC Columns, 4×250 mm
2. Solvent A: 20 mM sodium phosphate pH 7, 25% Acetonitrile; Solvent B: 20 mM Sodium phosphate, 1.5M Guanidine HCl pH 7, 25% acetonitrile
3. Flow Rate: 0.8 ml/min
4. Gradient:

| | Time | % A | % B |
|---|---|---|---|
| a. | 0.00 | 100 | 0 |
| b. | 3.00 | 100 | 0 |
| c. | 13.00 | 0 | 100 |
| d. | 23.00 | 0 | 100 |
| e. | 23.01 | 100 | 0 |
| f. | 33.00 | 100 | 0 |

### Strong Cation Exchange (SCX) Method 7

1. Column: Thermo Scientific, ProPac SCX-10 HPLC Columns, 4×250 mm
2. Solvent A: 10 mM sodium phosphate pH 7.5, 10% Ethanol; Solvent B: 10 mM Sodium phosphate, 1.5M NaCl pH 7.5, 10% ethanol
3. Flow Rate: 0.8 ml/min
4. Gradient:

| | Time | % A | % B |
|---|---|---|---|
| a. | 0.00 | 100 | 0 |
| b. | 3.00 | 100 | 0 |
| c. | 33.00 | 20 | 80 |
| d. | 33.10 | 0 | 100 |
| e. | 38.00 | 0 | 100 |
| f. | 38.10 | 100 | 0 |
| g. | 48.00 | 100 | 0 |

### Hydrophobic Interaction Chromatography (HIC) Method 4

1. Column: GE, HiScreen Butyl HP, 4.7 ml
2. Solvent A: 50 mM phosphate buffer, 0.8M Ammonium Sulfate, pH 7.0; Solvent B: 80% 50 mM phosphate buffer, 20% IPA, pH 7.0; Flow Rate: 1.0 ml/min
3. Gradient:

| | % A | % B | CV |
|---|---|---|---|
| a. | 100 | 0 | 4 |
| b. | 40 | 60 | 1 |
| c. | 40 | 60 | 4 |
| d. | 0 | 100 | 1 |
| e. | 0 | 100 | 3 |

### Size Exclusion Chromatography Method 1

1. Column: Phenomenex Yarra, 3 um SEC-3000, 300×7.8 mm
2. Mobile phase: 0.05 M KH2PO4, 0.2 M KCl ddH2O, pH 6.8, 10% isopropanol
3. Flow Rate: 1.0 ml/min for 15 mins
4. Detectors: UV absorbance at 220, 260 and 280 nm.

### Size Exclusion Chromatography (SEC) Method 2

1. Column: TOSOH Biosciences, TSKgelG3000SW XL, 7.8×300 mm, 5 μM
2. Mobile phase: 20 mM phosphate buffer, pH 7
3. Flow Rate: 1.0 ml/min for 20 mins

Size exclusion chromatography (SEC) method 2
1. Column: TOSOH Biosciences, TSKgelG3000SW, 21.5×600 mm, 5 mM
2. Mobile phase: PBS pH 7.4
3. Flow Rate: 1.0 ml/min for 180 mins

### Hydrophobic Interaction Chromatography (HIC) Method 1

1. Column: Thermo Scientific, MAbPac HIC-Butyl 5 μm, 4.5×100 mm
2. Solvent A: 100 mM phosphate buffer, 1.2 M sodium sulfate pH 7.0;
3. Solvent B: 80% 100 mM phosphate buffer, pH 7.0, 20% isopropanol
4. Flow rate=1 ml/min
5. Sample: dilute sample to 2.5 mg/ml antibody in PBS, inject 10 μl
6. Detectors: FLD excitation 280 nm, emission 345. UV absorbance, 220, 260 and 280 nm.
7. Gradient:

| | Time | % A | % B |
|---|---|---|---|
| a. | 0.0 | 100 | 0 |
| b. | 1.00 | 100 | 0 |
| c. | 1.50 | 100 | 0 |
| d. | 26.50 | 0 | 100 |
| e. | 28.50 | 0 | 100 |
| f. | 29.00 | 100 | 0 |
| g. | 33.00 | 100 | 0 |

### Hydrophobic Interaction Chromatography (HIC) Method 2

1. Column: GE HiScreen Butyl HP 4.7 mL CV
2. Column Temperature: 25° C.
3. Solvent A: 100 mM phosphate buffer, 0.7 M sodium sulfate pH 7.0;
4. Solvent B: 80% 100 mM phosphate buffer, pH 7.0, 20% isopropanol
5. Flow rate: 0.6 ml/min
6. Gradient:

| | % B | CV |
|---|---|---|
| a. | 0 | 1 |
| b. | 100 | 6.5 |
| c. | 100 | 5 |

### Hydrophobic Interaction Chromatography (HIC) Method 3

1. Column: Thermo Scientific, MAbPac HIC-20, 4.6 mm ID×10 cm, 5 μm
2. Solvent A: 100 mM phosphate buffer, 1.8 M Ammonium Sulfate, pH 7.0; Solvent B: 80% 100 mM phosphate buffer, 20% IPA, pH 7.0; Flow Rate: 0.7 ml/min
3. Gradient:

| | Time | % A | % B |
|---|---|---|---|
| a. | 0.00 | 100 | 0 |
| b. | 2.00 | 100 | 0 |
| c. | 22.00 | 0 | 100 |
| d. | 25.00 | 0 | 100 |
| e. | 26.00 | 100 | 0 |
| f. | 30.00 | 100 | 0 |

### Hydrophobic Interaction Chromatography (HIC) Method 4

1. Column: GE, HiScreen Butyl HP, 4.7 ml
2. Solvent A: 50 mM phosphate buffer, 0.8M Ammonium Sulfate, pH 7.0; Solvent B: 80% 50 mM phosphate buffer, 20% IPA, pH 7.0; Flow Rate: 1.0 ml/min
3. Gradient:

| | % A | % B | CV |
|---|---|---|---|
| a. | 100 | 0 | 4 |
| b. | 40 | 60 | 1 |
| c. | 40 | 60 | 4 |
| d. | 0 | 100 | 1 |
| e. | 0 | 100 | 3 |

### In Vivo Study Design and Sample Collection

Animals were dosed intravenously with PPMO-AOCs. Groups 1-2 contained four animals and groups 3-6 contained three animals. For dosing details, see Table 37 and Table 38.

TABLE 37
Summary of the design of the in vivo study to compare
the efficacy of PPMO-AOC compounds with Orientation 1

| | | | | | | | | Sample |
|---|---|---|---|---|---|---|---|---|
| | | | | | Compound | | | Collection |
| Animal | | | | | Info | | Dosing | Endpoints |
| and Group | | | | Animal | PMO | | Info | Harvest |
| Info | | | | Weight | Dose | | # | Time (d post |
| Group | Test Article | Strain | N | (kg) | (mg/kg) | DAR | Doses | dose) |
| 1 | PBS | C57- | 5 | 0.03 | — | — | 1 | 14 |
| | | BL10 | | | | | | |
| 2 | PBS | mdx | 5 | 0.03 | — | — | 1 | 14 |
| 3 | anti-mCD71 Ab- | mdx | 5 | 0.03 | 1.5 | 1.7 | 1 | 14 |
| | PMO23-(RXR)4XB | | | | | | | |
| | (DAR 1.7) | | | | | | | |
| 4 | anti-mCD71 Ab- | mdx | 5 | 0.03 | 5 | 1.7 | 1 | 14 |
| | PMO23-(RXR)4XB | | | | | | | |
| | (DAR 1.7) | | | | | | | |
| 5 | anti-mCD71 Ab- | mdx | 5 | 0.03 | 3.3 | 3.5 | 1 | 14 |
| | PMO23-(RXR)4XB | | | | | | | |
| | (DAR 3.5) | | | | | | | |
| 6 | anti-mCD71 Ab- | mdx | 5 | 0.03 | 10 | 3.5 | 1 | 14 |
| | PMO23-(RXR)4XB | | | | | | | |
| | (DAR 3.5) | | | | | | | |
| 7 | Isotype Ab-PMO23- | mdx | 5 | 0.03 | 10 | 3.5 | 1 | 14 |
| | (RXR)4XB (DAR | | | | | | | |
| | 3.5) | | | | | | | |

TABLE 38
Summary of the design of the in vivo study to compare
the efficacy of PPMO-AOC compounds with Orientation 2.

| | | | | | | | | Sample |
|---|---|---|---|---|---|---|---|---|
| | | | | | | | | Collection |
| Animal and Group Info | | | | | | | | Endpoints |
| | | | | Animal | Compound Info | | Dosing | Harvest |
| | | | | Weight | PMO Dose | | Info | Time (d |
| Group | Test Article | Strain | N | (kg) | (mg/kg) | DAR | # Doses | post dose) |
| 1 | PBS | C57- | 4 | — | — | 1 | 14 | |
| | | BL10 | | | | | | |
| 2 | PBS | mdx | 4 | — | — | 1 | 14 | |
| 3 | Anti-mCD71 Ab- | mdx | 3 | 5 | 2.1 | 1 | 14 | |
| | MC (RXR)4XB- | | | | | | | |
| | PMO23 | | | | | | | |
| 4 | Anti-mCD71 Ab- | mdx | 3 | 5 | 1.49 | 1 | 14 | |
| | MC Pip6a-PMO23 | | | | | | | |
| 5 | Anti-mCD71 Ab- | mdx | 3 | 5 | 1.85 | 1 | 14 | |
| | MC Pip6a -PMO23 | | | | | | | |
| 6 | Anti-mCD71 Ab- | mdx | 3 | 5 | 2.06 | 1 | 14 | |
| | MC Pip6a -PMO23 | | | | | | | |

Animals were harvested 14 days post dose. Two 20-30 mg tissue samples of Gastroc, Quad, Kidney, Liver, Diaphragm, and Heart were excised from each animal in all groups and stored in collection tubes with a 5 mm steel bead on 96 well plates at −80 C for subsequent exon skipping analysis and PMO/PPMO tissue concentration analysis. 50 mg tissue samples of Gastroc, Diaphragm, and Heart were excised from each animal in all groups and stored in cryo bags at −80° C. for future Western blot analysis.

### Exon Skipping Sample Homogenization

Tissue samples for exon skipping analysis were removed from −80° C. and allowed to thaw at room temperature for approximately 10 minutes. Foil covering was carefully removed from collection tubes. Any samples stuck near the tops of the collection tubes were poked down with individual pipette tips.
500 μL of Trizol was added to each 20-30 mg tissue sample. Tubes were capped tightly.
Plates were homogenized on the Tissue Lyser II for 5 minutes at 30 Hz and then cooled on dry ice for approximately 3 minutes. Plates were homogenized in the opposite orientation for another 5 minutes at 30 Hz and then cooled on dry ice for approximately 3 minutes. Samples were inspected for thorough homogenization. If samples were not thoroughly homogenized, a third round of homogenization was performed for 3 minutes at 30 Hz, and then samples were cooled on dry ice for 3 minutes.
After complete homogenization, plates were centrifuged for 5 minutes at 6,000 RCF to pull any large tissue pieces to the bottom of the tube.
50 μL of tissue homogenate from each sample was transferred to a well on a 96 well plate. Tubes were capped and stored at −80° C. for future use if necessary.

### Exon Skipping Sample RNA Isolation

RNA from exon skipping samples was isolated using the Zymo Research Direct-zol-96 RNA Kit (Zymo Research; R2056). An equal amount (50 μL) of 100% ethanol was added to the tissue homogenate. Ethanol and tissue homogenate were mixed thoroughly by pipetting up and down approximately 20 times.
All 100 μL of the mixture was transferred to a well on the Zymo-Spin I-96 plate mounted on top of a collection plate. The spin column plate was covered with a foil seal and centrifuged for 5 minutes at 6,000 RCF. Flow through was discarded and the spin column plate was placed on top of a new collection plate.
400 μL RNA Wash Buffer was added to each well of the spin plate. The spin column plate was covered with a foil seal and centrifuged for 5 minutes at 6,000 RCF. Flow through was discarded.
5 μL of DNase1 and 35 μL of Digestion Buffer were added to the center of each well.

The plate was incubated at room temperature for 15 minutes.
400 μl of Direct-zol RNA Pre Wash was added to each well. The spin column plate was covered with a foil seal and centrifuged for 5 minutes at 6,000 RCF. Flow through was discarded.
Previous step was repeated.
800 μL of RNA Wash Buffer was added to each well. Spin column plate was covered with a foil seal and centrifuged for 5 minutes at 6,000 RCF. Flow through was discarded.
Plates were centrifuged again for 5 minutes at 6,000 RCF to remove any remaining RNA Wash Buffer from previous steps.
A new 96 well PCR plate was sandwiched between the collection plate and spin column plate. 26 μL of DNase/RNase free water was added to the center of each well of the spin column plate. Plates were covered with a foil seal and centrifuged for 5 minutes at 6,000 RCF. The 96 well plate containing the eluted RNA was covered with a foil seal and stored at −80° C.

### Quantification and Normalization of RNA

RNA plates were removed from −80° C. and allowed to thaw on ice.
Plates were briefly centrifuged, mixed on shakers (two rounds of approximately 1500 RPM for 30 seconds), and briefly centrifuged again.
RNA concentration was quantified using the Tecan Infinite F200 Microplate Reader and the Tecan NanoQuant Plate. Tecan Infinite F200 Microplate Reader was blanked with nuclease free water. 2 μL of RNA from each sample were used for quantification.
All samples were normalized to 20 ng RNA/μL in nuclease free water, unless the RNA concentration of a sample happened to be <20 ng RNA/μL.

### Reverse Transcription

Normalized RNA (20 ng/μL) was converted to cDNA through reverse transcription (RT).
A master mix was prepared using the High-Capacity cDNA Reverse Transcription Kit (Thermo Fisher; #4368813) and Nuclease free water following Table 39 below.

TABLE 39
Reverse transcription reagent

| Reagent | 1 reaction (μL) |
|---|---|
| 10X RT Buffer | 2 |
| 25X dNTP | 0.8 |
| 10X Random Primer | 2 |
| RT Enzyme | 1 |
| Nuclease Free Water | 4.2 |
| Total | 10 |

10 μL of master mix was added to each well of a 96 well PCR plate.
10 μL of RNA (20 ng/μL) was added to each well, for a total volume of 20 μL in each well and a total input of 200 ng RNA in each well.
PCR plates were sealed with optical adhesive film, briefly centrifuged, mixed on a shaker (two rounds at approximately 1500 RPM for 30 seconds), and briefly centrifuged again.
PCR plates were placed in a thermocycler and programmed to incubate at 25° C. for 10 minutes, 37° C. for 120 minutes, 85° C. for 5 minutes, and then held at 4° C.
After completion of RT, plates were briefly centrifuged. cDNA was diluted 1:1 with water (20 μL of cDNA diluted with 20 μL of water). Plates were covered with a foil seal and stored at −20° C. until further use.
   qPCR for Exon Skipping Analysis
Exon skipping was analyzed via qPCR using Tagman assays to detect skipped and non-skipped transcripts. PPIB was also included in the analysis for normalization. Samples were ran in duplicate.
A qPCR master mix was prepared following Table 40 below.

TABLE 40
qPCR master mix

| Reagent | 1 reaction (uL) |
|---|---|
| Target Probe | 0.17 |
| Skipped: “d23”, FAM, 60X -or- | |
| Non-Skipped: “Ex22-23”, FAM, 60X | |
| PPIB Probe, VIC, 60X | 0.17 |
| Water | 0.66 |
| PCR Fast Master Mix | 5 |
| Total | 6 |

6 μL of master mix was added to each well of a 384 well qPCR plate.
4 μL of cDNA (diluted 1:1 with water) was added to each well, for a total qPCR reaction volume of 10 μL.
Plates were sealed with clear optical adhesive film. Plates were briefly centrifuged, mixed on a plate shaker (two rounds of approximately 2,000 RPM for 30 seconds), and briefly centrifuged again.
Plates were placed into the Applied Biosystems QuantStudio 6/7 Flex Real-Time PCR System, experimental properties were defined, and the following settings were applied:
   Polymerase activation at 95° C. for 20 seconds
   PCR 40 cycles
   Denature at 95° C. for 1 second
   Anneal/extend at 60° C. for 20 seconds.
For this study, qPCR thresholds for all targets were set to 0.2.
Data was exported into excel. Exon skipping was calculated as

$$\% skip = \frac{{100}^{*} [ {100}^{*} 2 \hat{} ( Δ ( {Ct}_{non skipped} - {CT}_{PPIB} ) - Δ ( {CT}_{skipped} - {CT}_{PPIB} ) ]}{100 + [ {100}^{*} 2 \hat{} ( Δ ( {Ct}_{non skipped} - {CT}_{PPIB} ) - Δ ( {CT}_{skipped} - {CT}_{PPIB} ) ]}$$

### PMO Tissue Concentration Sample Homogenization

Frozen tissue samples of 25-30 mg in 96-well homogenization tubes with 5 mm steel beads were allowed to thaw at RT for 15 minutes. Aluminum plate sealer was carefully removed, ensuring no tissue fragments were adhered.
400 μL of RIPA buffer was added to each well, then tubes were sealed with caps.
Samples were homogenized on TissueLyser II for 5 minutes at 30/s. The plate was rotated 180 degrees then homogenized again for 5 minutes.
The plate was centrifuged at 6000 rpm for 5 minutes. Tubes were inspected to determine if tissue homogenization was complete. An additional 5 minutes of homogenization was performed when needed.
After successful homogenization and centrifugation, caps were removed then 11 μL of Proteinase K (Promega MC5008) diluted to 8 mg/mL in RIPA buffer was added to each tube. Resulting working concentration was ˜200 μg/mL (varies slightly with respect to tissue concentration; for very large samples, PK concentration can be increased).
Tubes were re-capped. Plates were inverted several times to mix PK into homogenate.
Caps were secured by sandwiching a 96 well PCR plate between the tube caps and the lid of the plate and reinforcing with tape or a heavy weight. Samples were then incubated at 60° C. in oven. After 30 min, the plate was checked to ensure caps were still in place as high heat commonly leads to increased pressure and cap popping. Popped caps were re-secured. The plate was checked once more after 30 minutes, followed by overnight incubation.
The next day, plates were brought to room temperature and then tubes were inspected for sample digestion.
One final 3 minutes of homogenization at 30/s was performed to mix digested samples thoroughly followed by 5 minutes of centrifugation at 6000 rpm.
Caps were removed and sample supernatant was removed by pipetting midway into the tube, taking care to avoid touching the bottom. Samples were diluted into dilution buffer as needed.
Once necessary samples were removed, tubes were re-capped and the plate was stored at −20° C. For subsequent experimentation, the plate was removed from −20° C., heated at 60° C. for >1 hour, cooled to RT, then mixed and centrifuged as previously described.

### PMO Tissue Concentration ELISA

### Buffers

Wash buffer: standard 1×TBST (unfiltered) on plate washer system stored at room temperature.
   Dilute 10×TBST (Cell Signaling Technology #9997) to 1×TBST in MiliQ deionized water.
Dilution buffer: standard 1×TBST (filtered) with the addition of control muscle lysate or serum as required.
Hybridization buffer: 50 mM Tris-HCl, 1.15 M sodium chloride, pH 7.6, 0.3 mg/mL BSA, 0.10% v/v Triton X-100.
   Make 50 mM Tris-HCl, 1.15 M sodium chloride, pH 7.6. Filter and store at room temperature.
   Add 0.3 mg/mL BSA and 0.10% v/v Triton X-100 on day of experiment.
MNase buffer: 50 mM Tris-HCl (pH 8.5), 200 mM NaCl, 5 mM CaCl₂), 0.1 mg/mL BSA.
   Make 50 mM Tris-HCl (pH 8.5), 200 mM NaCl, 5 mM CaCl₂). Filter and store at room temperature. 10× stocks can be kept at −20° C. and diluted to 1× with miliQ deionized water.
   Add 0.1 mg/mL BSA to BSA-free stock on day of experiment.
Detection Antibody Buffer: SuperBlock Blocking Buffer in TBS (Thermo 37535)+0.25% v/v Tween-20; prepared fresh on day of experiment and kept at room temperature.
BSA 10× stock: 10 mg/mL BSA protein (Sigma A2934) dissolved in water was aliquoted and kept at −20° C. Free of preservatives. Kept at 4° C. once thawed.

### Plate Coating

Neutravidin coating buffer (Sigma C3041): the contents of one capsule were emptied into 100 mL MilliQ deionized water; 0.05 M carbonate-bicarbonate, pH 9.6.
Coating buffer should be used within 7 days.
NeutrAvidin Protein (Thermo 31000): 10 mg vial was dissolved in 10 mL DNase-/RNase-free (1 mg/mL) deionized water and stored at 4° C. protected from light.
Grenier Bio-One 96 well, F-bottom (Chimney Well), Black, FLUOTRAC, High Binding, Sterile plates (#655077)
Neutravidin protein at 1 mg/mL was diluted 1:1000 in coating buffer to yield 1 ug/mL working concentration (e.g. 11 uL NA in 11 mL buffer per plate).
100 uL was added to each well of the plate, which was then sealed and incubated at 37° C. for 2 hours.

### Standard Curves

For 10× standard curves, 1 μM (1,000 nM) stock of Gene Tools mouse Exon 23 PMO/PPMO was heated at 70° C. for 7 minutes, mixed well by pipetting, and diluted in dilution buffer starting at 100 nM (10×) followed by a 1:2 serial dilution for 12 points total.
   10× standard curve=100, 50, 2 5 . . . nM
   After adding 1:10 to appropriate matrix, 1× standard curve=10, 5, 2.5 . . . nM
Digested control plasma or homogenate for each tissue type was used to prepare the standard curve in the same percentage of plasma or homogenate as diluted samples.

### Hybridization

Complimentary DNA probe (custom from Eurofins) specific to mouse Exon 23 dystrophin (target) PMO/PPMO was diluted from 1 μM stock to 0.5 nM in hybridization buffer.
Equal volumes of PMO/PPMO standards or diluted experimental samples and 0.5 nM DNA probe solution were mixed in a PCR or deep well plate, sealed, then incubated at 37° C. for 30 minutes.
   E.g. 75 μL sample dilutions+75 uL 0.5 nM probe in 96-well PCR plate.
   100 μL of hybridized product was subsequently used in the assay.

### Probe-Plate Binding

After 2 hours at 37° C., Neutravidin solution was emptied from the plate followed by blotting on paper towels.
Wells were washed 3× with 200 μL of 1×TBST on plate washer (pre-set PMO ELISA protocol), followed by blotting on paper towels.
200 μL of SuperBlock Blocking Buffer in TBS (Thermo 37535) was added to wells for 5 minutes, followed by emptying and blotting on paper towels.
100 μL of hybridized PMO/PPMO-DNA probe standards or samples were added to wells.
Plates were sealed with plastic film then incubated at 37° C. for 30 minutes.

MNase DNA Cleavage
Micrococcal Nuclease (Thermo EN0181). Stock vials stored at −20° C.
Each well requires 150 μL of MNase; through experimentation, it was found that 0.9 U/well was an effective quantity; (0.9 U/well)/(150 μL/well)=0.006 U/pL=6 U/mL working concentration.
For each plate: (16 mL)*(6 U/mL)/(300 U/uL)=0.32 uL of 300 U/μL stock.
   i.e. 0.32 μL of 300 U/uL MNase in 16 mL MNase buffer per plate.
After 30 minutes at 37° C., hybridized sample solutions were emptied from the plate followed by blotting on paper towels.
Wells were washed 3× with 200 μL of 1×TBST on plate washer (pre-set PMO ELISA protocol) followed by blotting on paper towels.
150 μL of MNase solution was added to wells.
Plates were sealed with plastic film and then incubated at 37° C. for 1 hour.
Attophos AP Fluorescent Substrate System (Promega S1000) was warmed at 37° C. to be used later in the assay.

### Detection Antibody Binding

Monoclonal Anti-Digoxin-Alkaline Phosphatase antibody (Sigma A1054) was diluted 1:10,000 in detection antibody buffer (SuperBlock Blocking Buffer in TBS (Thermo 37535)+0.25% Tween-20).
For each plate: 1.1 μL Ab in 11 mL buffer.
After 1 hour at 37° C., MNase solution was emptied from the plate followed by blotting on paper towels.
Wells were washed 3× with 200 μL of 1×TBST on plate washer (pre-set PMO ELISA protocol), followed by blotting on paper towels.
100 μL of detection antibody solution was added to wells.
Plates were sealed with plastic film then incubated at 37° C. for 30 minutes.

### Alkaline Phosphatase Assay

AttoPhos AP Fluorescent Substrate System (Promega S1000).
60 mL of kit buffer was added to vial containing 36 mg of substrate and allowed at least 1 hour to dissolve; stored at 4° C. protected from light.
The reagent was warmed to 37 C in an incubator in a previous step.
After 30 minutes at 37° C., detection antibody solutions were emptied from the plate followed by blotting on paper towels.
Wells were washed twice with 3×250 μL of 1×TBST on plate washer (Run PMO ELISA Final Wash protocol two times).
100 μL of AttoPhos substrate solution was added to wells.
   Addition was staggered by 3 minutes between plates to allow for handling and read time.
Plates were returned to the oven at 37° C. (unsealed) and a stopwatch was started.
Plates were read on a Tecan Infinite F200 Microplate Reader
   Fluorescent Endpoint read with Ex=444 and Em=555 and AutoCutoff=550.
   COR96fb clear bottom; 30 flashes/read; Read from bottom; AttoPhos; gain 30; Z position 16100
   Read 60 minutes after addition of AttoPhos.

### Data Analysis for Standard Curves

Collected data were copied and pasted into Excel.

Results for standard curves were copied into GraphPad Prism. X values were transformed using X=log(X). Nonlinear regression analysis with 4 PL fit of the semi-log plot was performed.

In nearly all instances, R²>0.99 for this fit. Low and high standard values were inspected to assess plateauing on either end of the curve.

Data Analysis for Tissue samples
RFU values for samples were copied into Prism below standard values and interpolation was performed. Values outside of the low and high plateau points were excluded.
Interpolated values were transformed using X=10\hat{ }X to yield ELISA [PMO] pM.
ELISA [PMO] pM was multiplied by the appropriate dilution factor to yield Sample [PMO] pM.
Sample [PMO] pM was multiplied by (400+11+tissue weight)/(tissue weight) to yield Tissue [PMO] pM.
   400 for RIPA volume, 11 for Proteinase K volume, and tissue weight assuming 1 mg/μL density.
Tissue [PMO] pM was divided by 1000 to yield Tissue [PMO] nM.

### Exon 23 Skipping Results and Analysis

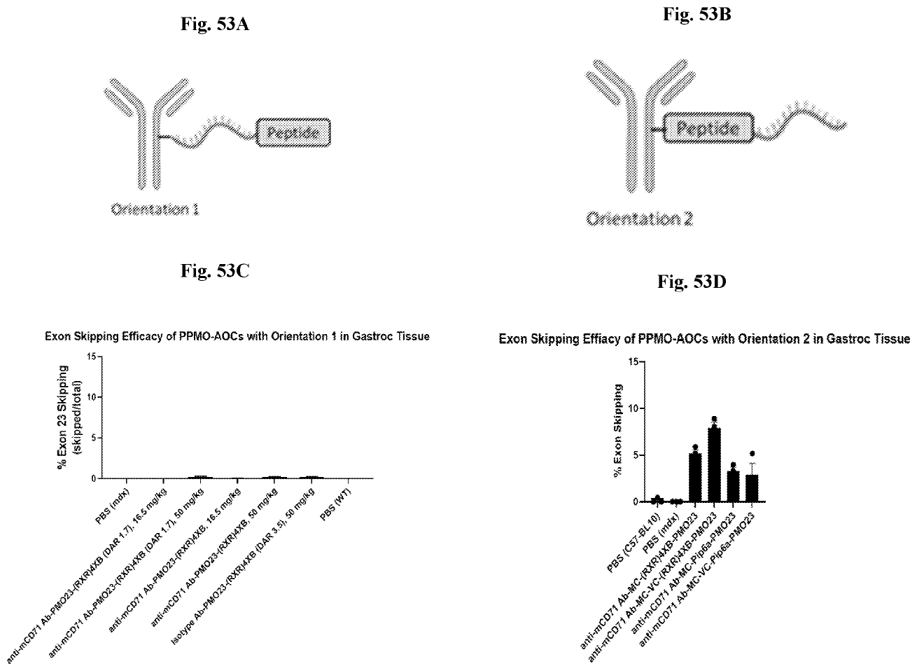

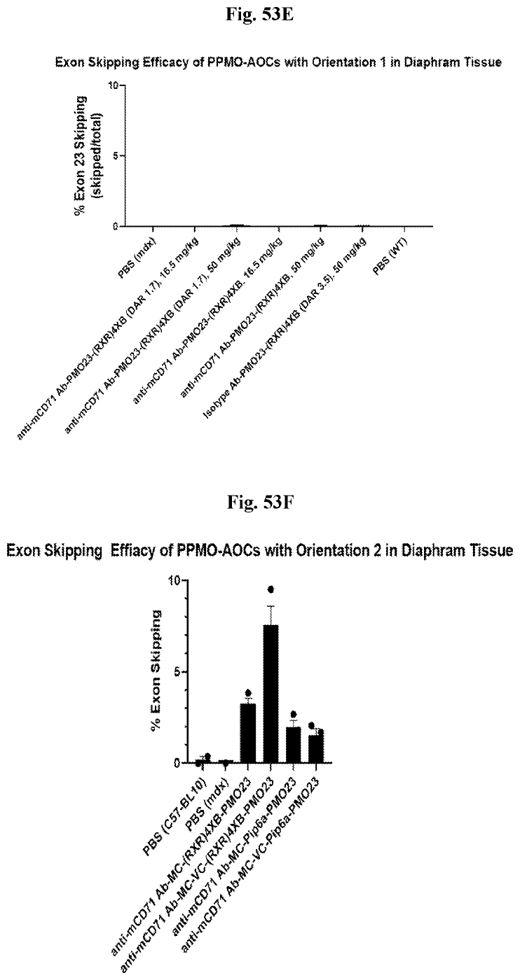

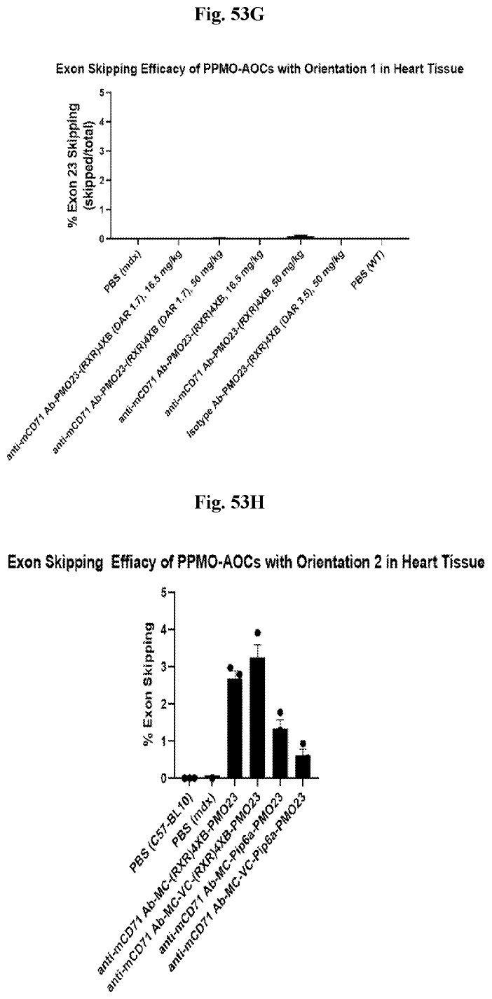

At Day 14 post-dose, the mdx mice administered with the 4 PPMO-AOCs with orientation 1 had little or no quantifiable levels of exon 23 skipping in any of the muscle tissue, including the gastrocnemius muscle (FIG. 53 C), diaphragm muscle (FIG. 53 E), or heart muscle (FIG. 53 G). However, all the mdx mice administered with the PPMO-AOCs with orientation 2 were able to induce exon 23 skipping in all muscle tissues (FIGS. 53 D, E, and F). The PPMO-AOC with orientation 2, anti-mCD71 Ab-MC-VC-(RXR)4XB-PMO23, had the best exon 23 skipping activity in all muscle tissues among the PPMO-AOCs with orientation 2. This PPMO-AOC, anti-mCD71 Ab-MC-VC-(RXR)4XB-PMO23, had more than 2-fold increase in exon 23 skipping activity in the diaphragm over the 3 other PPMO-AOCs. Overall, the PPMO-AOCs with orientation 2 are able to induce exon 23 skipping in all muscle tissues while the PPMO-AOCs with orientation 1 had no exon 23 skipping activity.

These results indicate that activities of PPMO-AOCs depend on the orientation of the PMO and peptide. The linear configuration of the PPMO-AOC with the peptide between the antibody and PMO of the PPMO-AOC (orientation 2) provides the optimal activity in muscle tissues.

### Exon 23 PMO Tissue Concentrations Results and Analysis

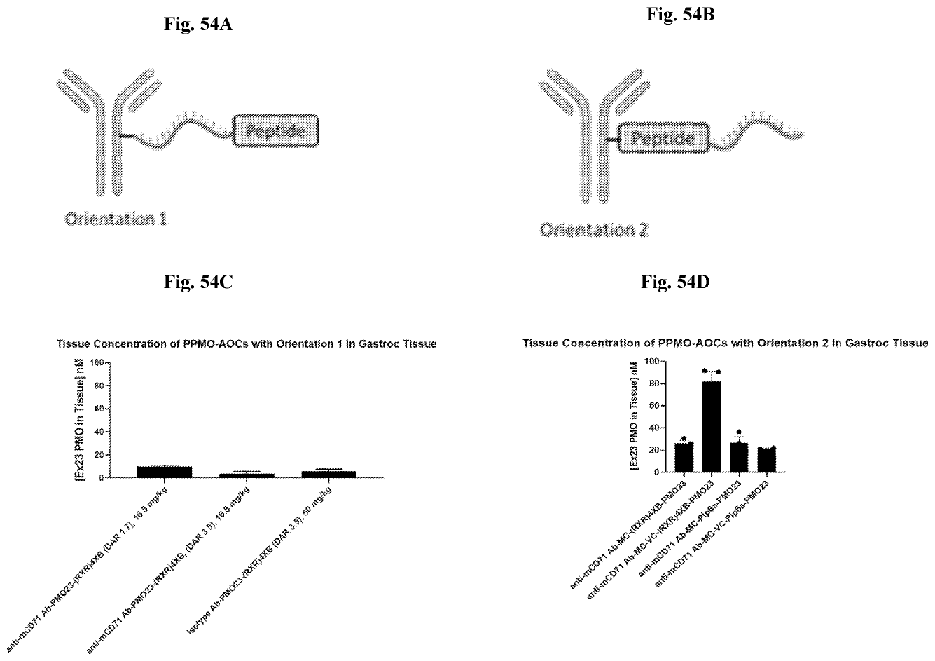

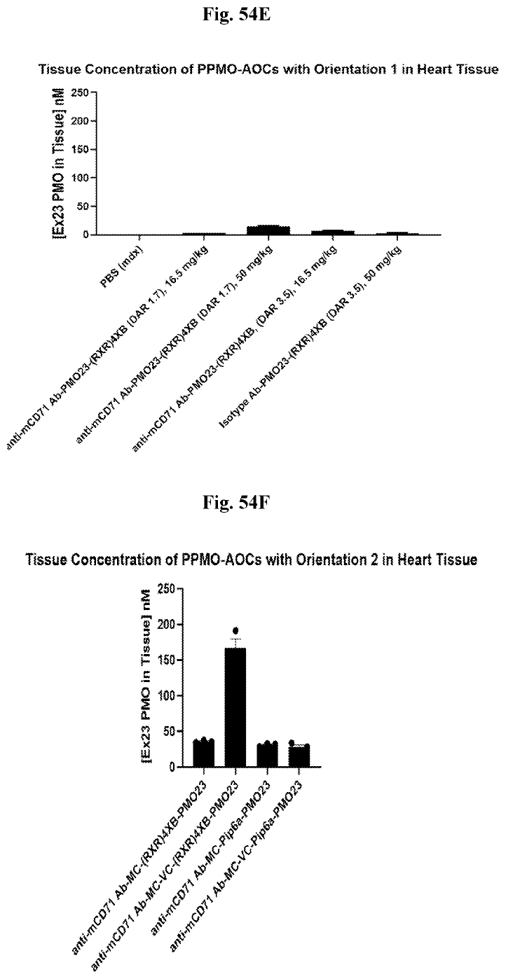

At Day 14 post-dose, the mdx mice administered with the PPMO-AOCs with orientation 1 had low concentrations of exon 23 PMO in the gastrocnemius muscle (FIG. 54 C), diaphragm muscle (FIG. 54 E). However, all the mdx mice administered with the PPMO-AOCs with orientation 2 had had much higher exon 23 PMO concentrations in muscles (FIGS. 54 D and F) than the ones for the PPMO-AOCs with orientation 1. The PPMO-AOCs with orientation 2 was able to deliver significantly more exon 23 PMO in muscle cells than the PPMO-AOCs with orientation 1. The PPMO-AOC with orientation 2, anti-mCD71 Ab-MC-VC-(RXR)₄XB-PMO23, delivered the most exon 23 PMO in muscle tissues compared to the PPMO-AOCs with orientation 2. This PPMO-AOC, anti-mCD71 Ab-MC-VC-(RXR)4XB-PM023, had more than 4-fold increase in exon 23 PMO concentrations in gastrocnemius and heart tissue over the 3 other PPMO-AOCs. Overall, the PPMO-AOCs with orientation 2 were able to deliver more exon 23 PMO to muscle tissues than the PPMO-AOCs with orientation 1.

These results indicate that tissue accumulation of exon 23 PMO depend on the orientation of the PPMO-AOC. The linear configuration of the PPMO-AOC with the peptide between the antibody and PMO of the PPMO-AOC (orientation 2) provides the most accumulation of exon 23 PMO in muscle tissues.

The examples and aspects described herein are for illustrative purposes only and various modifications or changes suggested to persons skilled in the art are to be included within the spirit and purview of this application and scope of the appended claims.

1.-63. (canceled)

64. An antibody-peptide-oligonucleotide conjugate (APOC) comprising:
A-(X₁-B-X₂-D)n Formula(V)
or
A-(X₁-D-X₂-B)n Formula (VI)
wherein,
A is an antibody or antigen binding fragment thereof,
B is a polynucleotide;
D is an endosomolytic peptide or a membrane penetrating peptide;
X₁ is a bond or a first non-polymeric linker;
X₂ is an optional bond or an optional second linker; and
n is an integer ≥1;
wherein the polynucleotide comprises at least one 2′ modified nucleotide, at least one modified internucleotide linkage, or at least one inverted abasic moiety.

65. The conjugate of claim 64, wherein the antibody or antigen binding fragment thereof comprises a humanized antibody or antigen binding fragment thereof, chimeric antibody or antigen binding fragment thereof, monoclonal antibody or antigen binding fragment thereof, monovalent Fab′, divalent Fab2, single-chain variable fragment (scFv), diabody, minibody, nanobody, single-domain antibody (sdAb), or camelid antibody or antigen binding fragment thereof.

66. The conjugate of claim 65, wherein the antibody or antigen binding fragment thereof binds to the transferrin receptor.

67. The conjugate of claim 64, wherein D is an endosomolytic peptide, and wherein the endosomolytic peptide is selected from INF7 and melittin.

68. The conjugate of claim 64, wherein D is a membrane penetrating peptide, and wherein the membrane penetrating peptide is selected from the group consisting of RRRRRRRRRRRR (SEQ ID NO:1000), GLAFLGFLGAAGSTMGAWSQPKKKRKV (SEQ ID NO:1001), RRIRPRPPRLPRPRPRPLPFPRPG (SEQ ID NO:1002), RKKRRQRRR (SEQ ID NO:1003), RRRRRRRRRR (SEQ ID NO:1004), GRPRESGKKRKRKRLKP (SEQ ID NO:1005), ALWKTLLKKVLKAPKKKRKV (SEQ ID NO: 1006), RRIPNRRPRR (SEQ ID NO:1007), TRRQRTRRARRNR (SEQ ID NO:1008), HARIKPTFRRLKWKYKGKFW (SEQ ID NO:1009), GIGAVLKVLTTGLPALISWIKRKRQQ (SEQ ID NO:1010), LRRERQSRLRRERQSR (SEQ ID NO:1011), RRRRRRRRR (SEQ ID NO:1012), RQIKIWFQNRRMKWKK (SEQ ID NO:1013), KRARNTEAARRSRARKLQRMKQ (SEQ ID NO:1014), RHIKIWFQNRRMKWKK (SEQ ID NO:1015), RRRRRRRR (SEQ ID NO:1016), KMTRAQRRAAARRNRWTAR (SEQ ID NO:1017), RGGRLSYSRRRFSTSTGR (SEQ ID NO:1018), KQINNWFINQRKRHWK (SEQ ID NO:1019), KLWMRWYSPTTRRYG (SEQ ID NO:1020), RRWWRRWRR (SEQ ID NO:1021), SQIKIWFQNKRAKIKK (SEQ ID NO:1022), GAYDLRRRERQSRLRRRERQSR (SEQ ID NO:1023), TRRNKRNRIQEQLNRK (SEQ ID NO:1024), GKRKKKGKLGKKRDP (SEQ ID NO:1025), RQVTIWFQNRRVKEKK (SEQ ID NO:1026), RLRWR (SEQ ID NO:1027), PPRPPRPPRPPRPPR (SEQ ID NO:1028), CAYHRLRRC (SEQ ID NO: 1029), SRRARRSPRHLGSG (SEQ ID NO:1030), PPRPPRPPRPPR (SEQ ID NO:1031), NAKTRRHERRRKLAIER (SEQ ID NO:1032), VKRGLKLRHVRPRVTRMDV (SEQ ID NO:1033), LYKKGPAKKGRPPLRGWFH (SEQ ID NO:1034), TAKTRYKARRAELIAERR (SEQ ID NO:1035), KGTYKKKLMRIPLKGT (SEQ ID NO:1036), PPRPPRPPR (SEQ ID NO:1037), RASKRDGSWVKKLHRILE (SEQ ID NO:1038), TRSSRAGLQWPVGRVHRLLRK (SEQ ID NO:1039), FKIYDKKVRTRVVKH (SEQ ID NO:1040), VRLPPPVRLPPPVRLPPP (SEQ ID NO: 1041), GPFHFYQFLFPPV (SEQ ID NO:1042), PLILLRLLRGQF (SEQ ID NO:1043), YTAIAWVKAFIRKLRK (SEQ ID NO:1044), KETWWETWWTEWSQPKKRKV (SEQ ID NO:1045), LIRLWSHLIHIWFQNRRLKWKKK (SEQ ID NO:1046), VDKGSYLPRPTPPRPIYNRN (SEQ ID NO:1047), MDAQTRRRERRAEKQAQWKAAN (SEQ ID NO:1048), GSPWGLQHHPPRT (SEQ ID NO:1049), KLALKALKALKAALKLA (SEQ ID NO:1050), IPALK (SEQ ID NO:1051), VPALR (SEQ ID NO:1052), LLIILRRRIRKQAHAHSK (SEQ ID NO:1053), IAWVKAFIRKLRKGPLG (SEQ ID NO:1054), AAVLLPVLLAAPVQRKRQKLP (SEQ ID NO:1055), TSPLNIHNGQKL (SEQ ID NO:1056), VPTLK (SEQ ID NO: 1057), VSALK (SEQ ID NO:1058), (RXR)4XB (SEQ ID NO:1065), and RXRRBRRXRYQFLIRXRBRXRB (SEQ ID NO: 1067).

69. The conjugate of claim 68, wherein the membrane penetrating peptide is RRRRRRRR (SEQ ID NO:1016), (RXR)4XB (SEQ ID NO:1065), or RXRRBRRXRYQFLIRXRBRXRB (SEQ ID NO: 1067).

70. The conjugate of claim 64, wherein D-X2 is conjugated to the 5′ end of B or the 3′ end of B.

71. The conjugate of claim 64, wherein the at least one 2′ modified nucleotide comprises 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA) modified nucleotide, locked nucleic acid (LNA) or ethylene nucleic acid (ENA), wherein the at least one modified internucleotide linkage comprises a phosphorothioate linkage or a phosphorodithioate linkage, and wherein the at least one inverted abasic moiety is at least one terminus. comprises.

72. The conjugate of claim 64, wherein the polynucleotide comprises a single-stranded nucleotide, and wherein the single-stranded nucleotide comprises an antisense oligonucleotide (ASO) or phosphorodiamidate morpholino oligonucleotide (PMO).

73. The conjugate of claim 64, wherein the polynucleotide comprises a sequence having at least 90%, 95%, 99%, or 100% sequence identity to a sequence selected from SEQ ID NOs:225-227, 252-263, 268-272, 352-427, 768-827, and 939-972.

74. The conjugate of claim 64, wherein X₁ is a non-polymeric linker group, a bond, a cleavable linker (e.g., a maleimide group with a-valine-citrulline linker), or a non-cleavable linker (e.g., a maleimide group).

75. The conjugate of claim 64, wherein X₂ is a C₁-C₆ alkyl group, or a homobifunctional linker or a heterobifunctional linker that is optionally conjugated to a C₁-C₆ alkyl group.

76. The conjugate of claim 64, further comprising C, wherein C is a polymer,

77. The conjugate of claim 76, wherein C is conjugated to the molecule of Formula (VI) according to Formula (VII): 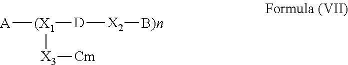
wherein,
A is an antibody or antigen binding fragment thereof,
B is a polynucleotide;
D is an endosomolytic peptide or a membrane penetrating peptide
C is a polymer;
X₁ is a bond or first non-polymeric linker;
X₂ is an optional bond or optional second linker;
X₃ is a bond or third linker;
n is an integer ≥1;
m is an integer ≥1; and
wherein the polynucleotide comprises at least one 2′ modified nucleotide, at least one modified internucleotide linkage, or at least one inverted abasic moiety;
wherein A and C are not attached to B at the same terminus; and
wherein D is conjugated anywhere on A or C or to a terminus of B.

78. The conjugate of claim 77, wherein X₃ is a C₁-C₆ alkyl group, a homobifunctional linker, or a heterobifunctional linker.

79. A method of treating a muscular dystrophy in a subject in need thereof, comprising:
administering to the subject the antibody-peptide-oligonucleotide conjugate of claim 64;
wherein the antibody-peptide-oligonucleotide conjugate induces splicing out of an exon to generate a mRNA transcript, and wherein the mRNA transcript encodes a truncated protein, thereby treating the muscular dystrophy in the subject.

80. The method of claim 79, wherein the muscular dystrophy is Duchenne muscular dystrophy.

81. A method of inducing exon skipping in a subject in need thereof, comprising:
administering to the subject the antibody-peptide-oligonucleotide conjugate of claim 64;
wherein the antibody-peptide-oligonucleotide conjugate induces exon skipping in the pre-mRNA transcript to generate a mRNA transcript, and wherein the mRNA transcript encodes a truncated protein.


SEQUENCE LISTING (1072 sequences)

SEQ ID NO: 1 - protein, 24 aa, Artificial Sequence
  Description of Artificial Sequence: Synthetic peptide
CGIFGEIEELIEEGLENLIDWGNA

SEQ ID NO: 2 - protein, 24 aa, Artificial Sequence
  Description of Artificial Sequence: Synthetic peptide
GLFEAIEGFIENGWEGMIDGWYGC

SEQ ID NO: 3 - protein, 27 aa, Artificial Sequence
  Description of Artificial Sequence: Synthetic peptide
GLFEAIEGFIENGWEGMIWDYGSGSCG

SEQ ID NO: 4 - protein, 23 aa, Artificial Sequence
  Description of Artificial Sequence: Synthetic peptide
GLFEAIEGFIENGWEGMIDGWYG

SEQ ID NO: 5 - protein, 27 aa, Artificial Sequence
  Description of Artificial Sequence: Synthetic peptide
GLFEAIEGFIENGWEGMIWDYGSGSCK

SEQ ID NO: 6 - protein, 27 aa, Artificial Sequence
  Description of Artificial Sequence: Synthetic peptide
CLIGAILKVLATGLPTLISWIKNKRKQ

SEQ ID NO: 7 - protein, 26 aa, Artificial Sequence
  Description of Artificial Sequence: Synthetic peptide
GIGAVLKVLTTGLPALISWIKRKRQQ

SEQ ID NO: 8 - protein, 13 aa, Artificial Sequence
  Description of Artificial Sequence: Synthetic peptide
IFGAIAGLLKNIF

SEQ ID NO: 9 - protein, 18 aa, Artificial Sequence
  Description of Artificial Sequence: Synthetic peptide
FFGHLFKLATKIIPSLFQ

SEQ ID NO: 10 - protein, 21 aa, Artificial Sequence
  Description of Artificial Sequence: Synthetic peptide
KETWWETWWTEWSQPKKKRKV

SEQ ID NO: 11 - protein, 19 aa, Artificial Sequence
  Description of Artificial Sequence: Synthetic peptide
LLIILRRRRIRKQAHAHSK

SEQ ID NO: 12 - protein, 26 aa, Artificial Sequence
  Description of Artificial Sequence: Synthetic peptide
DPKGDPKGVTVTVTVTVTGKGDPKPD

SEQ ID NO: 13 - protein, 17 aa, Artificial Sequence
  Description of Artificial Sequence: Synthetic peptide
CSIPPEVKFNKPFVYLI

SEQ ID NO: 14 - protein, 27 aa, Artificial Sequence
  Description of Artificial Sequence: Synthetic peptide
GWTLNSAGYLLGKINLKALAALAKKIL

SEQ ID NO: 15 - protein, 21 aa, Artificial Sequence
  Description of Artificial Sequence: Synthetic peptide
AGYLLGKINLKALAALAKKIL

SEQ ID NO: 16 - protein, 17 aa, Artificial Sequence
  Description of Artificial Sequence: Synthetic peptide
GALFLGFLGAAGSTMGA

SEQ ID NO: 17 - protein, 20 aa, Artificial Sequence
  Description of Artificial Sequence: Synthetic peptide
HGLASTLTRWAHYNALIRAF

SEQ ID NO: 18 - protein, 20 aa, Artificial Sequence
  Description of Artificial Sequence: Synthetic peptide
GLWRALWRLLRSLWRLLWRA

SEQ ID NO: 19 - protein, 30 aa, Artificial Sequence
  Description of Artificial Sequence: Synthetic polypeptide
WEAALAEALAEALAEHLAEALAEALEALAA

SEQ ID NO: 20 - protein, 24 aa, Artificial Sequence
  Description of Artificial Sequence: Synthetic peptide
GLFEAIEGFIENGWEGMIDGWYGC

SEQ ID NO: 21 - protein, 23 aa, Artificial Sequence
  Description of Artificial Sequence: Synthetic peptide
GLFGAIAGFIENGWEGMIDGWYG

SEQ ID NO: 22 - protein, 36 aa, Artificial Sequence
  Description of Artificial Sequence: Synthetic polypeptide
GLFGAIAGFIENGWEGMIDGRQIKIWFQNRRMKWKK

SEQ ID NO: 23 - protein, 26 aa, Artificial Sequence
  Description of Artificial Sequence: Synthetic peptide
GLFGAIAGFIENGWEGMIDGSSKKKK

SEQ ID NO: 24 - protein, 24 aa, Artificial Sequence
  Description of Artificial Sequence: Synthetic peptide
GLFEAIAGFIENGWEGMIDGGGYC

SEQ ID NO: 25 - protein, 23 aa, Artificial Sequence
  Description of Artificial Sequence: Synthetic peptide
GLFHAIAHFIHGGWHGLIHGWYG

SEQ ID NO: 26 - protein, 30 aa, Artificial Sequence
  Description of Artificial Sequence: Synthetic polypeptide
GLFEAIEGFIENGWEGLAEALAEALEALAA

SEQ ID NO: 27 - protein, 29 aa, Artificial Sequence
  Description of Artificial Sequence: Synthetic peptide
KWKLFKKIGAVLKVLTTGYGRKKRRQRRR

SEQ ID NO: 28 - DNA, 25 bp, Artificial Sequence
  Description of Artificial Sequence: Synthetic oligonucleotide
ggccaaacctcggcttacctgaaat

SEQ ID NO: 29 - RNA, 20 nt, Artificial Sequence
  Description of Artificial Sequence: Synthetic oligonucleotide
ggccaaaccucggcuuaccu

SEQ ID NO: 30 - DNA, 22 bp, Artificial Sequence
  Description of Artificial Sequence: Synthetic primer
cagaattctgccaattgctgag

SEQ ID NO: 31 - DNA, 20 bp, Artificial Sequence
  Description of Artificial Sequence: Synthetic primer
ttcttcagcttgtgtcatcc

SEQ ID NO: 32 - DNA, 20 bp, Artificial Sequence
  Description of Artificial Sequence: Synthetic primer
acccagtctaccaccctatc

SEQ ID NO: 33 - DNA, 22 bp, Artificial Sequence
  Description of Artificial Sequence: Synthetic primer
ctctttatcttctgcccacctt

SEQ ID NO: 34 - DNA, 21 bp, Artificial Sequence
  Description of Artificial Sequence: Synthetic primer
gcgctatcaggagacaatgag

SEQ ID NO: 35 - DNA, 26 bp, Artificial Sequence
  Description of Artificial Sequence: Synthetic primer
gtttttatgtgattctgtaatttccc

SEQ ID NO: 36 - DNA, 24 bp, Artificial Sequence
  Description of Artificial Sequence: Synthetic probe
ctctctgtaccttatcttagtgtt

SEQ ID NO: 37 - DNA, 20 bp, Artificial Sequence
  Description of Artificial Sequence: Synthetic primer
tggaggagagactcgggaaa

SEQ ID NO: 38 - DNA, 23 bp, Artificial Sequence
  Description of Artificial Sequence: Synthetic primer
ttgaagccattttgttgctcttt

SEQ ID NO: 39 - DNA, 16 bp, Artificial Sequence
  Description of Artificial Sequence: Synthetic probe
acaggctctgcaaagt

SEQ ID NO: 40 - DNA, 24 bp, Artificial Sequence
  Description of Artificial Sequence: Synthetic primer
aacagatgacaactactgccgaaa

SEQ ID NO: 41 - DNA, 23 bp, Artificial Sequence
  Description of Artificial Sequence: Synthetic primer
ttggctctgatagggtggtagac

SEQ ID NO: 42 - DNA, 14 bp, Artificial Sequence
  Description of Artificial Sequence: Synthetic probe
cttgttgaaaaccc

SEQ ID NO: 43 - DNA, 23 bp, Artificial Sequence
  Description of Artificial Sequence: Synthetic primer
tgagggtgttaatgctgaaagta

SEQ ID NO: 44 - DNA, 20 bp, Artificial Sequence
  Description of Artificial Sequence: Synthetic primer
caccaactgggaggaaagtt

SEQ ID NO: 45 - DNA, 25 bp, Artificial Sequence
  Description of Artificial Sequence: Synthetic oligonucleotide
ggccaaacctcggcttacctgaaat

SEQ ID NO: 46 - DNA, 28 bp, Artificial Sequence
  Description of Artificial Sequence: Synthetic oligonucleotide
agcccatcttctcctggtcctgggaagg

SEQ ID NO: 47 - DNA, 25 bp, Artificial Sequence
  Description of Artificial Sequence: Synthetic oligonucleotide
atcctctttggtaacctcacctcac

SEQ ID NO: 48 - DNA, 25 bp, Artificial Sequence
  Description of Artificial Sequence: Synthetic oligonucleotide
tcgtccacaaaatgattctgaatta

SEQ ID NO: 49 - DNA, 25 bp, Artificial Sequence
  Description of Artificial Sequence: Synthetic oligonucleotide
cggtgtgtgtatcattctctagtgt

SEQ ID NO: 50 - DNA, 22 bp, Artificial Sequence
  Description of Artificial Sequence: Synthetic primer
cctggaaacagctcctaacatc

SEQ ID NO: 51 - DNA, 21 bp, Artificial Sequence
  Description of Artificial Sequence: Synthetic primer
cagtcaagcccaaagtctctc

SEQ ID NO: 52 - DNA, 20 bp, Artificial Sequence
  Description of Artificial Sequence: Synthetic primer
ctagtgcccttgttttcaga

SEQ ID NO: 53 - DNA, 19 bp, Artificial Sequence
  Description of Artificial Sequence: Synthetic primer
aggatctaccactgatggg

SEQ ID NO: 54 - RNA, 31 nt, Artificial Sequence
  Description of Artificial Sequence: Synthetic oligonucleotide
gccugagcugaucugcuggcaucuugcaguu

SEQ ID NO: 55 - RNA, 57 nt, Artificial Sequence
  Description of Artificial Sequence: Synthetic oligonucleotide
gcagaauucgauccaccggcuguucaagccugagcugaucugcucgcaucuugcagu

SEQ ID NO: 56 - RNA, 22 nt, Artificial Sequence
  Description of Artificial Sequence: Synthetic oligonucleotide
cagcaguaguugucaucugcuc

SEQ ID NO: 57 - RNA, 29 nt, Artificial Sequence
  Description of Artificial Sequence: Synthetic oligonucleotide
cacaaagucugcauccaggaacauggguc

SEQ ID NO: 58 - RNA, 22 nt, Artificial Sequence
  Description of Artificial Sequence: Synthetic oligonucleotide
cugcaauuccccgagucucugc

SEQ ID NO: 59 - RNA, 23 nt, Artificial Sequence
  Description of Artificial Sequence: Synthetic oligonucleotide
cucauaccuucugcuugaugauc

SEQ ID NO: 60 - RNA, 30 nt, Artificial Sequence
  Description of Artificial Sequence: Synthetic oligonucleotide
uccaacuggggacgccucuguuccaaaucc

SEQ ID NO: 61 - RNA, 24 nt, Homo sapiens
gauaggugguaucaacaucuguaa

SEQ ID NO: 62 - RNA, 21 nt, Homo sapiens
gauaggugguaucaacaucug

SEQ ID NO: 63 - RNA, 25 nt, Homo sapiens
gauaggugguaucaacaucuguaag

SEQ ID NO: 64 - RNA, 20 nt, Homo sapiens
ggugguaucaacaucuguaa

SEQ ID NO: 65 - RNA, 20 nt, Homo sapiens
guaucaacaucuguaagcac

SEQ ID NO: 66 - RNA, 23 nt, Homo sapiens
ugcauguuccagucguugugugg

SEQ ID NO: 67 - RNA, 25 nt, Homo sapiens
cacuauuccagucaaauaggucugg

SEQ ID NO: 68 - RNA, 25 nt, Homo sapiens
auuuaccaaccuucaggaucgagua

SEQ ID NO: 69 - RNA, 21 nt, Homo sapiens
ggccuaaaacacauacacaua

SEQ ID NO: 70 - RNA, 20 nt, Canis sp.
cauuuuugaccuacaugugg

SEQ ID NO: 71 - RNA, 20 nt, Canis sp.
uuugaccuacauguggaaag

SEQ ID NO: 72 - RNA, 26 nt, Canis sp.
uacauuuuugaccuacauguggaaag

SEQ ID NO: 73 - RNA, 22 nt, Canis sp.
auuuuugaccuacaugggaaag

SEQ ID NO: 74 - RNA, 23 nt, Canis sp.
uacgaguugauugucggacccag

SEQ ID NO: 75 - RNA, 25 nt, Canis sp.
guggucuccuuaccuaugacugugg

SEQ ID NO: 76 - RNA, 17 nt, Canis sp.
ggucuccuuaccuauga

SEQ ID NO: 77 - RNA, 24 nt, Homo sapiens
ugucucaguaaucuucuuaccuau

SEQ ID NO: 78 - RNA, 24 nt, Homo sapiens
ucuuaccuaugacuauggaugaga

SEQ ID NO: 79 - RNA, 20 nt, Homo sapiens
gcaugaacucuuguggaucc

SEQ ID NO: 80 - RNA, 20 nt, Homo sapiens
ccaggguacuacuuacauua

SEQ ID NO: 81 - RNA, 21 nt, Homo sapiens
aucgugugucacagcauccag

SEQ ID NO: 82 - RNA, 30 nt, Homo sapiens
uguucagggcaugaacucuuguggauccuu

SEQ ID NO: 83 - RNA, 31 nt, Homo sapiens
uaggaggcgccucccauccuguaggucacug

SEQ ID NO: 84 - RNA, 31 nt, Homo sapiens
aggucuaggaggcgccucccauccuguaggu

SEQ ID NO: 85 - RNA, 25 nt, Homo sapiens
gcgccucccauccuguaggucacug

SEQ ID NO: 86 - RNA, 26 nt, Homo sapiens
cuucgaggaggucuaggaggcgccuc

SEQ ID NO: 87 - RNA, 21 nt, Homo sapiens
cucccauccuguaggucacug

SEQ ID NO: 88 - RNA, 22 nt, Homo sapiens
uaccaguuuuugcccugucagg

SEQ ID NO: 89 - RNA, 26 nt, Homo sapiens
ucaauaugcugcuucccaaacugaaa

SEQ ID NO: 90 - RNA, 25 nt, Homo sapiens
cuaggaggcgccucccauccuguag

SEQ ID NO: 91 - RNA, 31 nt, Homo sapiens
uuaugauuuccaucuacgaugucaguacuuc

SEQ ID NO: 92 - RNA, 31 nt, Homo sapiens
cuuaccugccaguggaggauuauauuccaaa

SEQ ID NO: 93 - RNA, 25 nt, Homo sapiens
caucaggauucuuaccugccagugg

SEQ ID NO: 94 - RNA, 25 nt, Homo sapiens
cgaugucaguacuuccaauauucac

SEQ ID NO: 95 - RNA, 18 nt, Homo sapiens
accauucaucaggauucu

SEQ ID NO: 96 - RNA, 18 nt, Homo sapiens
accugccaguggaggauu

SEQ ID NO: 97 - RNA, 27 nt, Homo sapiens
ccaauauucacuaaaucaaccuguuaa

SEQ ID NO: 98 - RNA, 30 nt, Homo sapiens
caggauuguuaccugccaguggaggauuau

SEQ ID NO: 99 - RNA, 31 nt, Homo sapiens
acgaugucaguacuuccaauauucacuaaau

SEQ ID NO: 100 - RNA, 31 nt, Homo sapiens
auuuccaucuacgaugucaguacuuccaaua

SEQ ID NO: 101 - RNA, 21 nt, Homo sapiens
caggagcuuccaaaugcugca

SEQ ID NO: 102 - RNA, 29 nt, Homo sapiens
cuugucuucaggagcuuccaaaugcugca

SEQ ID NO: 103 - RNA, 22 nt, Homo sapiens
uccucagcagaaagaagccacg

SEQ ID NO: 104 - RNA, 20 nt, Homo sapiens
uuagaaaucucuccuugugc

SEQ ID NO: 105 - RNA, 20 nt, Homo sapiens
uaaauuggguguuacacaau

SEQ ID NO: 106 - RNA, 24 nt, Homo sapiens
cccugaggcauucccaucuugaau

SEQ ID NO: 107 - RNA, 20 nt, Homo sapiens
aggacuuacuugcuuuguuu

SEQ ID NO: 108 - RNA, 23 nt, Homo sapiens
cuugaauuuaggagauucaucug

SEQ ID NO: 109 - RNA, 23 nt, Homo sapiens
caucuucugauaauuuuccuguu

SEQ ID NO: 110 - RNA, 24 nt, Homo sapiens
ucuucuguuuuuguuagccaguca

SEQ ID NO: 111 - RNA, 20 nt, Homo sapiens
ucuauguaaacugaaaauuu

SEQ ID NO: 112 - RNA, 20 nt, Homo sapiens
uucuggagauccauuaaaac

SEQ ID NO: 113 - RNA, 24 nt, Homo sapiens
cagcaguugcgugaucuccacuag

SEQ ID NO: 114 - RNA, 21 nt, Homo sapiens
uucaucaacuaccaccaccau

SEQ ID NO: 115 - RNA, 25 nt, Homo sapiens
cuaagcaaaauaaucugaccuuaag

SEQ ID NO: 116 - RNA, 28 nt, Homo sapiens
cuuguaaaagaacccagcggucuucugu

SEQ ID NO: 117 - RNA, 22 nt, Homo sapiens
caucuacagauguuugcccauc

SEQ ID NO: 118 - RNA, 23 nt, Homo sapiens
gaaggaugucuuguaaaagaacc

SEQ ID NO: 119 - RNA, 20 nt, Homo sapiens
accuguucuucaguaagacg

SEQ ID NO: 120 - RNA, 24 nt, Homo sapiens
caugacacaccuguucuucaguaa

SEQ ID NO: 121 - RNA, 20 nt, Homo sapiens
cauuugagaaggaugucuug

SEQ ID NO: 122 - RNA, 24 nt, Homo sapiens
aucucccaauaccuggagaagaga

SEQ ID NO: 123 - RNA, 31 nt, Homo sapiens
gccaugcacuaaaaaggcacugcaagacauu

SEQ ID NO: 124 - RNA, 24 nt, Homo sapiens
ucuuuaaagccaguugugugaauc

SEQ ID NO: 125 - RNA, 21 nt, Homo sapiens
uuucugaaagccaugcacuaa

SEQ ID NO: 126 - RNA, 25 nt, Homo sapiens
guacauacggccaguuuuugaagac

SEQ ID NO: 127 - RNA, 31 nt, Homo sapiens
cuagauccgcuuuuaaaaccuguuaaaacaa

SEQ ID NO: 128 - RNA, 31 nt, Homo sapiens
ucuuuucuagauccgcuuuuaaaaccuguua

SEQ ID NO: 129 - RNA, 25 nt, Homo sapiens
cuagauccgcuuuuaaaaccuguua

SEQ ID NO: 130 - RNA, 23 nt, Homo sapiens
ccgucuucugggucacugacuua

SEQ ID NO: 131 - RNA, 26 nt, Homo sapiens
cuagauccgcuuuuaaaaccuguuaa

SEQ ID NO: 132 - RNA, 20 nt, Homo sapiens
ccgcuuuuaaaaccuguuaa

SEQ ID NO: 133 - RNA, 26 nt, Homo sapiens
uggauugcuuuuucuuuucuagaucc

SEQ ID NO: 134 - RNA, 25 nt, Homo sapiens
caugcuuccgucuucugggucacug

SEQ ID NO: 135 - RNA, 23 nt, Homo sapiens
gaucuuguuugagugaauacagu

SEQ ID NO: 136 - RNA, 22 nt, Homo sapiens
guuauccagccaugcuuccguc

SEQ ID NO: 137 - RNA, 25 nt, Homo sapiens
ugauaauugguaucacuaaccugug

SEQ ID NO: 138 - RNA, 22 nt, Homo sapiens
guaucacuaaccugugcuguac

SEQ ID NO: 139 - RNA, 19 nt, Homo sapiens
cugcuggcaucuugcaguu

SEQ ID NO: 140 - RNA, 31 nt, Homo sapiens; identical to SEQ ID NO: 54

SEQ ID NO: 141 - RNA, 28 nt, Homo sapiens
cuggcagaauucgauccaccggcuguuc

SEQ ID NO: 142 - RNA, 22 nt, Homo sapiens
cagcaguaguugucaucugcuc

SEQ ID NO: 143 - RNA, 19 nt, Homo sapiens
ugauggggugguggguugg

SEQ ID NO: 144 - RNA, 25 nt, Homo sapiens
aucugcauuaacacccucuagaaag

SEQ ID NO: 145 - RNA, 24 nt, Homo sapiens
ccggcuguucaguuguucugaggc

SEQ ID NO: 146 - RNA, 28 nt, Homo sapiens
aucugcauuaacacccucuagaaagaaa

SEQ ID NO: 147 - RNA, 28 nt, Homo sapiens
gaaggagaagagauucuuaccuuacaaa

SEQ ID NO: 148 - RNA, 20 nt, Homo sapiens
auucgauccaccggcuguuc

SEQ ID NO: 149 - RNA, 20 nt, Homo sapiens
cagcaguaguugucaucugc

SEQ ID NO: 150 - RNA, 22 nt, Homo sapiens
gccgguugacuucauccugugc

SEQ ID NO: 151 - RNA, 22 nt, Homo sapiens
cugcauccaggaacaugggucc

SEQ ID NO: 152 - RNA, 23 nt, Homo sapiens
gucugcauccaggaacauggguc

SEQ ID NO: 153 - RNA, 24 nt, Homo sapiens
guugaagaucugauagccgguuga

SEQ ID NO: 154 - RNA, 24 nt, Homo sapiens
uacuuacugucuguagcucuuucu

SEQ ID NO: 155 - RNA, 24 nt, Homo sapiens
cacucauggucuccugauagcgca

SEQ ID NO: 156 - RNA, 22 nt, Homo sapiens
cugcaauuccccgagucucugc

SEQ ID NO: 157 - RNA, 23 nt, Homo sapiens
acugcuggacccauguccugaug

SEQ ID NO: 158 - RNA, 21 nt, Homo sapiens
cuaaguugagguauggagagu

SEQ ID NO: 159 - RNA, 23 nt, Homo sapiens
uauucacagaccugcaauucccc

SEQ ID NO: 160 - RNA, 26 nt, Homo sapiens
acaguggugcugagauaguauaggcc

SEQ ID NO: 161 - RNA, 22 nt, Homo sapiens
uaggccacuuuguugcucuugc

SEQ ID NO: 162 - RNA, 19 nt, Homo sapiens
uucagagggcgcuuucuuc

SEQ ID NO: 163 - RNA, 23 nt, Homo sapiens
gggcaggccauuccuccuucaga

SEQ ID NO: 164 - RNA, 24 nt, Homo sapiens
ucuucaggguuuguaugugauucu

SEQ ID NO: 165 - RNA, 27 nt, Homo sapiens
cugggcugaauugucugaauaucacug

SEQ ID NO: 166 - RNA, 26 nt, Homo sapiens
cuguuggcacaugugaucccacugag

SEQ ID NO: 167 - RNA, 24 nt, Homo sapiens
gucuauaccuguuggcacauguga

SEQ ID NO: 168 - RNA, 25 nt, Homo sapiens
ugcuuucuguaauucaucuggaguu

SEQ ID NO: 169 - RNA, 26 nt, Homo sapiens
ccuccuuucuggcauagaccuuccac

SEQ ID NO: 170 - RNA, 25 nt, Homo sapiens
ugugucauccauucgugcaucucug

SEQ ID NO: 171 - RNA, 25 nt, Homo sapiens
uuaaggccucuugugcuacaggugg

SEQ ID NO: 172 - RNA, 23 nt, Homo sapiens
ggggcucuucuuuagcucucuga

SEQ ID NO: 173 - RNA, 22 nt, Homo sapiens
gacuuccaaagucuugcauuuc

SEQ ID NO: 174 - RNA, 24 nt, Homo sapiens
gccaacaugcccaaacuuccuaag

SEQ ID NO: 175 - RNA, 26 nt, Homo sapiens
cagagauuuccucagcuccgccagga

SEQ ID NO: 176 - RNA, 21 nt, Homo sapiens
cuuacaucuagcaccucagag

SEQ ID NO: 177 - RNA, 25 nt, Homo sapiens
uccgccaucuguuagggucugugcc

SEQ ID NO: 178 - RNA, 25 nt, Homo sapiens
auuuggguuauccucugaaugucgc

SEQ ID NO: 179 - RNA, 22 nt, Homo sapiens
cauaccucuucauguaguuccc

SEQ ID NO: 180 - RNA, 26 nt, Homo sapiens
cauuugagcugcguccaccuugucug

SEQ ID NO: 181 - RNA, 26 nt, Homo sapiens
uccugggcagacuggaugcucuguuc

SEQ ID NO: 182 - RNA, 23 nt, Homo sapiens
uugccugggcuuccugaggcauu

SEQ ID NO: 183 - RNA, 24 nt, Homo sapiens
uucugaaauaacauauaccugugc

SEQ ID NO: 184 - RNA, 25 nt, Homo sapiens
uaguuucugaaauaacauauaccug

SEQ ID NO: 185 - RNA, 21 nt, Homo sapiens
gacuugucaaaucagauugga

SEQ ID NO: 186 - RNA, 24 nt, Homo sapiens
guuucugaaauaacauauaccugu

SEQ ID NO: 187 - RNA, 20 nt, Homo sapiens
caccagaaauacauaccaca

SEQ ID NO: 188 - RNA, 20 nt, Homo sapiens
caaugauuuagcugugacug

SEQ ID NO: 189 - RNA, 23 nt, Homo sapiens
cgaaacuucauggagacaucuug

SEQ ID NO: 190 - RNA, 25 nt, Homo sapiens
cuuguagacgcugcucaaaauuggc

SEQ ID NO: 191 - RNA, 20 nt, Homo sapiens
caugcacacaccuuugcucc

SEQ ID NO: 192 - RNA, 24 nt, Homo sapiens
ucuguacaaucugacguccagucu

SEQ ID NO: 193 - RNA, 27 nt, Homo sapiens
gucuuuaucaccauuuccacuucagac

SEQ ID NO: 194 - RNA, 25 nt, Homo sapiens
ccgucugcuuuuucuguacaaucug

SEQ ID NO: 195 - RNA, 22 nt, Homo sapiens
uccauaucuguagcugccagcc

SEQ ID NO: 196 - RNA, 23 nt, Homo sapiens
ccaggcaacuucagaauccaaau

SEQ ID NO: 197 - RNA, 30 nt, Homo sapiens
uuucuguuaccugaaaagaauuauaaugaa

SEQ ID NO: 198 - RNA, 25 nt, Homo sapiens
cauucauuuccuuucgcaucuuacg

SEQ ID NO: 199 - RNA, 26 nt, Homo sapiens
ugaucucuuugucaauuccauaucug

SEQ ID NO: 200 - RNA, 30 nt, Homo sapiens
uucagugauauagguuuuaccuuuccccag

SEQ ID NO: 201 - RNA, 26 nt, Homo sapiens
cuguagcugccagccauucugucaag

SEQ ID NO: 202 - RNA, 21 nt, Homo sapiens
ucuucugcucgggaggugaca

SEQ ID NO: 203 - RNA, 20 nt, Homo sapiens
ccaguuacuauucagaagac

SEQ ID NO: 204 - RNA, 20 nt, Homo sapiens
ucuucaggugcaccuucugu

SEQ ID NO: 205 - RNA, 25 nt, Homo sapiens
ugugaugugguccacauucugguca

SEQ ID NO: 206 - RNA, 20 nt, Homo sapiens
ccauguguuucugguauucc

SEQ ID NO: 207 - RNA, 25 nt, Homo sapiens
cguguagaguccaccuuugggcgua

SEQ ID NO: 208 - RNA, 24 nt, Homo sapiens
uacuaauuuccugcaguggucacc

SEQ ID NO: 209 - RNA, 24 nt, Homo sapiens
uucugugugaaauggcugcaaauc

SEQ ID NO: 210 - RNA, 20 nt, Homo sapiens
ccuucaaaggaauggaggcc

SEQ ID NO: 211 - RNA, 25 nt, Homo sapiens
ugcugaauuucagccuccagugguu

SEQ ID NO: 212 - RNA, 25 nt, Homo sapiens
ugaagucuuccucuuucagauucac

SEQ ID NO: 213 - RNA, 24 nt, Homo sapiens
cuggcuuucucucaucugugauuc

SEQ ID NO: 214 - RNA, 20 nt, Homo sapiens
guuguaaguugucuccucuu

SEQ ID NO: 215 - RNA, 20 nt, Homo sapiens
uugucuguaacagcugcugu

SEQ ID NO: 216 - RNA, 20 nt, Homo sapiens
gcucuaauaccuugagagca

SEQ ID NO: 217 - RNA, 22 nt, Homo sapiens
cuuugagaccucaaauccuguu

SEQ ID NO: 218 - RNA, 25 nt, Homo sapiens
cuuuauuuuccuuucaucucugggc

SEQ ID NO: 219 - RNA, 27 nt, Homo sapiens
aucguuucuucacggacagugugcugg

SEQ ID NO: 220 - RNA, 24 nt, Homo sapiens
gggcuugugagacaugagugauuu

SEQ ID NO: 221 - RNA, 22 nt, Homo sapiens
accuucagaggacuccucuugc

SEQ ID NO: 222 - RNA, 25 nt, Homo sapiens
uauguguuaccuacccuugucgguc

SEQ ID NO: 223 - RNA, 20 nt, Homo sapiens
ggagagagcuuccuguagcu

SEQ ID NO: 224 - RNA, 23 nt, Homo sapiens
ucacccuuuccacaggcguugca

SEQ ID NO: 225 - RNA, 20 nt, Homo sapiens
uuugugucuuucugagaaac

SEQ ID NO: 226 - RNA, 20 nt, Homo sapiens
aaagacuuaccuuaagauac

SEQ ID NO: 227 - RNA, 20 nt, Homo sapiens
aucugucaaaucgccugcag

SEQ ID NO: 228 - RNA, 20 nt, Homo sapiens
uuaccuugacuugcucaagc

SEQ ID NO: 229 - RNA, 20 nt, Homo sapiens
uccagguucaagugggauac

SEQ ID NO: 230 - RNA, 25 nt, Homo sapiens
gcucuucugggcuuaugggagcacu

SEQ ID NO: 231 - RNA, 27 nt, Homo sapiens
accuuuauccacuggagauuugucugc

SEQ ID NO: 232 - RNA, 21 nt, Homo sapiens
uuccaccaguaacugaaacag

SEQ ID NO: 233 - RNA, 29 nt, Homo sapiens
ccacucagagcucagaucuucuaacuucc

SEQ ID NO: 234 - RNA, 27 nt, Homo sapiens
cuuccacucagagcucagaucuucuaa

SEQ ID NO: 235 - RNA, 25 nt, Homo sapiens
gggauccaguauacuuacaggcucc

SEQ ID NO: 236 - RNA, 26 nt, Homo sapiens
accagaguaacagucugaguaggagc

SEQ ID NO: 237 - RNA, 23 nt, Homo sapiens
cucauaccuucugcuugaugauc

SEQ ID NO: 238 - RNA, 24 nt, Homo sapiens
uucuguccaagcccgguugaaauc

SEQ ID NO: 239 - RNA, 30 nt, Homo sapiens
acaucaaggaagauggcauuucuaguuugg

SEQ ID NO: 240 - RNA, 25 nt, Homo sapiens
acaucaaggaagauggcauuucuag

SEQ ID NO: 241 - RNA, 30 nt, Homo sapiens
cuccaacaucaaggaagauggcauuucuag

SEQ ID NO: 242 - RNA, 25 nt, Homo sapiens
aucauuuuuucucauaccuucugcu

SEQ ID NO: 243 - RNA, 36 nt, Homo sapiens
aucauuuuuucucauaccuucugcuaggagcuaaaa

SEQ ID NO: 244 - skipped

SEQ ID NO: 245 - RNA, 21 nt, Homo sapiens
cacccaccaucacccucugug

SEQ ID NO: 246 - RNA, 22 nt, Homo sapiens
aucaucucguugauauccucaa

SEQ ID NO: 247 - RNA, 21 nt, Homo sapiens
uccugcauuguugccuguaag

SEQ ID NO: 248 - RNA, 30 nt, Homo sapiens; identical to SEQ ID NO: 60

SEQ ID NO: 249 - RNA, 21 nt, Homo sapiens
acuggggacgccucuguucca

SEQ ID NO: 250 - RNA, 20 nt, Homo sapiens
ccguaaugauuguucuagcc

SEQ ID NO: 251 - RNA, 20 nt, Homo sapiens
uguuaaaaaacuuacuucga

SEQ ID NO: 252 - RNA, 25 nt, Homo sapiens
cauucaacuguugccuccgguucug

SEQ ID NO: 253 - RNA, 24 nt, Homo sapiens
cuguugccuccgguucugaaggug

SEQ ID NO: 254 - RNA, 31 nt, Homo sapiens
cauucaacuguugccuccgguucugaaggug

SEQ ID NO: 255 - RNA, 21 nt, Homo sapiens
uacuaaccuugguuucuguga

SEQ ID NO: 256 - RNA, 25 nt, Homo sapiens
cugaagguguucuuguacuucaucc

SEQ ID NO: 257 - RNA, 27 nt, Homo sapiens
uguauagggacccuccuuccaugacuc

SEQ ID NO: 258 - RNA, 25 nt, Homo sapiens
cuaaccuugguuucugugauuuucu

SEQ ID NO: 259 - RNA, 27 nt, Homo sapiens
gguaucuuugauacuaaccuugguuuc

SEQ ID NO: 260 - RNA, 22 nt, Homo sapiens
auucuuucaacuagaauaaaag

SEQ ID NO: 261 - RNA, 25 nt, Homo sapiens
gauucugaauucuuucaacuagaau

SEQ ID NO: 262 - RNA, 20 nt, Homo sapiens
aucccacugauucugaauuc

SEQ ID NO: 263 - RNA, 22 nt, Homo sapiens
uuggcucuggccuguccuaaga

SEQ ID NO: 264 - RNA, 30 nt, Homo sapiens
cucuuuuccagguucaagugggauacuagc

SEQ ID NO: 265 - RNA, 31 nt, Homo sapiens
caagcuuuucuuuuaguugcugcucuuuucc

SEQ ID NO: 266 - RNA, 30 nt, Homo sapiens
uauucuuuuguucuucuagccuggagaaag

SEQ ID NO: 267 - RNA, 28 nt, Homo sapiens
cugcuuccuccaaccauaaaacaaauuc

SEQ ID NO: 268 - RNA, 26 nt, Homo sapiens
ccaaugccauccuggaguuccuguaa

SEQ ID NO: 269 - RNA, 20 nt, Homo sapiens
uccuguagaauacuggcauc

SEQ ID NO: 270 - RNA, 27 nt, Homo sapiens
ugcagaccuccugccaccgcagauuca

SEQ ID NO: 271 - RNA, 20 nt, Homo sapiens
cuaccucuuuuuucugucug

SEQ ID NO: 272 - RNA, 20 nt, Homo sapiens
uguuuuugaggauugcugaa

SEQ ID NO: 273 - DNA, 20 bp, Artificial Sequence
  Description of Artificial Sequence: Synthetic oligonucleotide
tggttcttacccagccgccg

SEQ ID NO: 274 - DNA, 20 bp, Artificial Sequence
  Description of Artificial Sequence: Synthetic oligonucleotide
gttattctttagaatggtgc

SEQ ID NO: 275 - DNA, 20 bp, Artificial Sequence
  Description of Artificial Sequence: Synthetic oligonucleotide
tgctattaccttaacccaga

SEQ ID NO: 276 - DNA, 27 bp, Artificial Sequence
  Description of Artificial Sequence: Synthetic oligonucleotide
ctgtgcttaccgggttttccacctccc

SEQ ID NO: 277 - DNA, 25 bp, Artificial Sequence
  Description of Artificial Sequence: Synthetic oligonucleotide
atcgtcgtgactgtctgttggaggg

SEQ ID NO: 278 - DNA, 20 bp, Artificial Sequence
  Description of Artificial Sequence: Synthetic oligonucleotide
gctcacgttgaggggcatcg

SEQ ID NO: 279 - DNA, 20 bp, Artificial Sequence
  Description of Artificial Sequence: Synthetic oligonucleotide
acgttgaggggcatcgtcgc

SEQ ID NO: 280 - RNA, 30 nt, Artificial Sequence
  Description of Artificial Sequence: Synthetic oligonucleotide
ggggcaucgucgugacugucuguuggaggg

SEQ ID NO: 281 - RNA, 22 nt, Artificial Sequence
  Description of Artificial Sequence: Synthetic oligonucleotide
cgucgugacugucuguuggagg

SEQ ID NO: 282 - DNA, 22 bp, Artificial Sequence
  Description of Artificial Sequence: Synthetic oligonucleotide
cgtcgtgactgtctgttggagg

SEQ ID NO: 283 - RNA, 28 nt, Artificial Sequence
  Description of Artificial Sequence: Synthetic oligonucleotide
ggcaucgucgcgggaggcugcuggagcg

SEQ ID NO: 284 - RNA, 28 nt, Artificial Sequence
  Description of Artificial Sequence: Synthetic oligonucleotide
ccgcgacauaggacggagagcagagccc

SEQ ID NO: 285 - DNA, 20 bp, Artificial Sequence
  Description of Artificial Sequence: Synthetic oligonucleotide
actgtgagggcgatcgctgc

SEQ ID NO: 286 - DNA, 20 bp, Artificial Sequence
  Description of Artificial Sequence: Synthetic oligonucleotide
acgatgagtggcatagtcgc

SEQ ID NO: 287 - DNA, 20 bp, Artificial Sequence
  Description of Artificial Sequence: Synthetic oligonucleotide
ggcatcgtcgcgggaggctg

SEQ ID NO: 288 - DNA, 20 bp, Artificial Sequence
  Description of Artificial Sequence: Synthetic oligonucleotide
gggcatcgtcgcgggaggct

SEQ ID NO: 289 - DNA, 20 bp, Artificial Sequence
  Description of Artificial Sequence: Synthetic oligonucleotide
ggggcatcgtcgcgggaggc

SEQ ID NO: 290 - DNA, 20 bp, Artificial Sequence
  Description of Artificial Sequence: Synthetic oligonucleotide
aggggcatcgtcgcgggagg

SEQ ID NO: 291 - DNA, 20 bp, Artificial Sequence
  Description of Artificial Sequence: Synthetic oligonucleotide
gaggggcatcgtcgcgggag

SEQ ID NO: 292 - DNA, 20 bp, Artificial Sequence
  Description of Artificial Sequence: Synthetic oligonucleotide
tgaggggcatcgtcgcggga

SEQ ID NO: 293 - DNA, 20 bp, Artificial Sequence
  Description of Artificial Sequence: Synthetic oligonucleotide
ttgaggggcatcgtcgcggg

SEQ ID NO: 294 - DNA, 20 bp, Artificial Sequence
  Description of Artificial Sequence: Synthetic oligonucleotide
gttgaggggcatcgtcgcgg

SEQ ID NO: 295 - DNA, 20 bp, Artificial Sequence
  Description of Artificial Sequence: Synthetic oligonucleotide
cgttgaggggcatcgtcgcg

SEQ ID NO: 296 - DNA, 20 bp, Artificial Sequence
  Description of Artificial Sequence: Synthetic oligonucleotide
acgttgaggggcatcgtcgc

SEQ ID NO: 297 - DNA, 20 bp, Artificial Sequence
  Description of Artificial Sequence: Synthetic oligonucleotide
aacgttgaggggcatcgtcg

SEQ ID NO: 298 - DNA, 20 bp, Artificial Sequence
  Description of Artificial Sequence: Synthetic oligonucleotide
taacgttgaggggcatcgtc

SEQ ID NO: 299 - DNA, 20 bp, Artificial Sequence
  Description of Artificial Sequence: Synthetic oligonucleotide
ctaacgttgaggggcatcgt

SEQ ID NO: 300 - DNA, 20 bp, Artificial Sequence
  Description of Artificial Sequence: Synthetic oligonucleotide
gctaacgttgaggggcatcg

SEQ ID NO: 301 - DNA, 20 bp, Artificial Sequence
  Description of Artificial Sequence: Synthetic oligonucleotide
agctaacgttgaggggcatc

SEQ ID NO: 302 - DNA, 20 bp, Artificial Sequence
  Description of Artificial Sequence: Synthetic oligonucleotide
aagctaacgttgaggggcat

SEQ ID NO: 303 - DNA, 20 bp, Artificial Sequence
  Description of Artificial Sequence: Synthetic oligonucleotide
gaagctaacgttgaggggca

SEQ ID NO: 304 - DNA, 20 bp, Rattus sp.
ctccgcaatgctgaaaggtg

SEQ ID NO: 305 - RNA, 24 nt, Rattus sp.
ggcgugccucaaacaugguggcgg

SEQ ID NO: 306 - DNA, 27 bp, Rattus sp.
ctgtgcttaccgggttttccacctccc

SEQ ID NO: 307 - DNA, 25 bp, Rattus sp.
atcgtcgtgactgtctgttggaggg

SEQ ID NO: 308 - DNA, 20 bp, Rattus sp.
gctcacgttgaggggcatcg

SEQ ID NO: 309 - DNA, 20 bp, Rattus sp.
ggtcactcaccggtagagaa

SEQ ID NO: 310 - DNA, 20 bp, Rattus sp.
gggttccaagtctataaagg

SEQ ID NO: 311 - DNA, 14 bp, Homo sapiens
tgtgtcttttccag

SEQ ID NO: 312 - DNA, 23 bp, Homo sapiens
tttggagactgccagggaccatg

SEQ ID NO: 313 - DNA, 20 bp, Homo sapiens
catggtccctggcagtctcc

SEQ ID NO: 314 - DNA, 36 bp, Homo sapiens
tcaatgggcaaaacatggtccctggcagtctccaaa

SEQ ID NO: 315 - DNA, 15 bp, Homo sapiens
tttgtgttctcccag

SEQ ID NO: 316 - DNA, 23 bp, Homo sapiens
ggaaacagaagtacctgtgcgcc

SEQ ID NO: 317 - DNA, 18 bp, Homo sapiens
ggcgcacaggtacttctg

SEQ ID NO: 318 - DNA, 36 bp, Homo sapiens
aatcatttctgctggcgcacaggtacttctgtttcc

SEQ ID NO: 319 - DNA, 18 bp, Homo sapiens
cccctgcagcacgcgggt

SEQ ID NO: 320 - DNA, 37 bp, Homo sapiens
gaggcagggccggcaggaccccctgcagcacgcgggt

SEQ ID NO: 321 - DNA, 20 bp, Homo sapiens
ggcatcgtcgcgggaggctg

SEQ ID NO: 322 - DNA, 20 bp, Homo sapiens
gggcatcgtcgcgggaggct

SEQ ID NO: 323 - DNA, 20 bp, Homo sapiens
ggggcatcgtcgcgggaggc

SEQ ID NO: 324 - DNA, 20 bp, Homo sapiens
aggggcatcgtcgcgggagg

SEQ ID NO: 325 - DNA, 20 bp, Homo sapiens
gaggggcatcgtcgcgggag

SEQ ID NO: 326 - DNA, 20 bp, Homo sapiens
tgaggggcatcgtcgcggga

SEQ ID NO: 327 - DNA, 20 bp, Homo sapiens
ttgaggggcatcgtcgcggg

SEQ ID NO: 328 - DNA, 20 bp, Homo sapiens
gttgaggggcatcgtcgcgg

SEQ ID NO: 329 - DNA, 20 bp, Homo sapiens
cgttgaggggcatcgtcgcg

SEQ ID NO: 330 - DNA, 20 bp, Homo sapiens
acgttgaggggcatcgtcgc

SEQ ID NO: 331 - DNA, 20 bp, Homo sapiens
aacgttgaggggcatcgtcg

SEQ ID NO: 332 - DNA, 20 bp, Homo sapiens
taacgttgaggggcatcgtc

SEQ ID NO: 333 - DNA, 20 bp, Homo sapiens
ctaacgttgaggggcatcgt

SEQ ID NO: 334 - DNA, 20 bp, Homo sapiens
gctaacgttgaggggcatcg

SEQ ID NO: 335 - DNA, 20 bp, Homo sapiens
agctaacgttgaggggcatc

SEQ ID NO: 336 - DNA, 20 bp, Homo sapiens
aagctaacgttgaggggcat

SEQ ID NO: 337 - DNA, 20 bp, Homo sapiens
gaagctaacgttgaggggca

SEQ ID NO: 338 - DNA, 20 bp, Homo sapiens
tcctcatcttcttgttcctc

SEQ ID NO: 339 - DNA, 36 bp, Homo sapiens
aacaacatcgatttcttcctcatcttcttgttcctc

SEQ ID NO: 340 - DNA, 18 bp, Homo sapiens
cccggaaggcagtctggc

SEQ ID NO: 341 - DNA, 36 bp, Homo sapiens
tcctccatggcagtgacccggaaggcagtctggctg

SEQ ID NO: 342 - DNA, 19 bp, Homo sapiens
ctactggccgctgaagggc

SEQ ID NO: 343 - DNA, 36 bp, Homo sapiens
gctcaaagtcagatgctactggccgctgaagggctt

SEQ ID NO: 344 - DNA, 27 bp, Human immunodeficiency virus 1
tcgtcggtctctccgcttcttcttgcc

SEQ ID NO: 345 - DNA, 20 bp, Human immunodeficiency virus 1
ctctggtggtgggtaagggt

SEQ ID NO: 346 - DNA, 37 bp, Human immunodeficiency virus 1
cgggtctgtcgggttccctctggtggtgggtaagggt

SEQ ID NO: 347 - RNA, 30 nt, Rattus sp.; identical to SEQ ID NO: 280

SEQ ID NO: 348 - RNA, 22 nt, Rattus sp.
cgucgugacugucuguuggagg

SEQ ID NO: 349 - DNA, 22 bp, Rattus sp.
cgtcgtgactgtctgttggagg

SEQ ID NO: 350 - RNA, 28 nt, Homo sapiens
ggcaucgucgcgggaggcugcuggagcg

SEQ ID NO: 351 - RNA, 28 nt, Rattus sp.
ccgcgacauaggacggagagcagagccc

SEQ ID NO: 352 - DNA, 25 bp, Artificial Sequence
  Description of Artificial Sequence: Synthetic oligonucleotide
ctgcaggtaaaagcatatggatcaa

SEQ ID NO: 353 - DNA, 25 bp, Artificial Sequence
  Description of Artificial Sequence: Synthetic oligonucleotide
atcgcctgcaggtaaaagcatatgg

SEQ ID NO: 354 - DNA, 25 bp, Artificial Sequence
  Description of Artificial Sequence: Synthetic oligonucleotide
gtcaaatcgcctgcaggtaaaagca

SEQ ID NO: 355 - DNA, 25 bp, Artificial Sequence
  Description of Artificial Sequence: Synthetic oligonucleotide
gatctgtcaaatcgcctgcaggtaa

SEQ ID NO: 356 - DNA, 25 bp, Artificial Sequence
  Description of Artificial Sequence: Synthetic oligonucleotide
caacagatctgtcaaatcgcctgca

SEQ ID NO: 357 - DNA, 25 bp, Artificial Sequence
  Description of Artificial Sequence: Synthetic oligonucleotide
tttctcaacagatctgtcaaatcgc

SEQ ID NO: 358 - DNA, 25 bp, Artificial Sequence
  Description of Artificial Sequence: Synthetic oligonucleotide
ccatttctcaacagatctgtcaaat

SEQ ID NO: 359 - DNA, 25 bp, Artificial Sequence
  Description of Artificial Sequence: Synthetic oligonucleotide
ataatgaaaacgccgccatttctca

SEQ ID NO: 360 - DNA, 25 bp, Artificial Sequence
  Description of Artificial Sequence: Synthetic oligonucleotide
aaatatctttatatcataatgaaaa

SEQ ID NO: 361 - DNA, 25 bp, Artificial Sequence
  Description of Artificial Sequence: Synthetic oligonucleotide
tgttagccactgattaaatatcttt

SEQ ID NO: 362 - DNA, 25 bp, Artificial Sequence
  Description of Artificial Sequence: Synthetic oligonucleotide
aaactgttcagcttctgttagccac

SEQ ID NO: 363 - DNA, 25 bp, Artificial Sequence
  Description of Artificial Sequence: Synthetic oligonucleotide
ttgtgtctttctgagaaactgttca

SEQ ID NO: 364 - DNA, 25 bp, Artificial Sequence
  Description of Artificial Sequence: Synthetic oligonucleotide
ccaattctcaggaatttgtgtcttt

SEQ ID NO: 365 - DNA, 25 bp, Artificial Sequence
  Description of Artificial Sequence: Synthetic oligonucleotide
gtatttagcatgttcccaattctca

SEQ ID NO: 366 - DNA, 25 bp, Artificial Sequence
  Description of Artificial Sequence: Synthetic oligonucleotide
cttaagataccatttgtatttagca

SEQ ID NO: 367 - DNA, 25 bp, Artificial Sequence
  Description of Artificial Sequence: Synthetic oligonucleotide
cttaccttaagataccatttgtatt

SEQ ID NO: 368 - DNA, 25 bp, Artificial Sequence
  Description of Artificial Sequence: Synthetic oligonucleotide
aaagacttaccttaagataccattt

SEQ ID NO: 369 - DNA, 25 bp, Artificial Sequence
  Description of Artificial Sequence: Synthetic oligonucleotide
aaatcaaagacttaccttaagatac

SEQ ID NO: 370 - DNA, 25 bp, Artificial Sequence
  Description of Artificial Sequence: Synthetic oligonucleotide
aaaacaaatcaaagacttaccttaa

SEQ ID NO: 371 - DNA, 25 bp, Artificial Sequence
  Description of Artificial Sequence: Synthetic oligonucleotide
tcgaaaaaacaaatcaaagacttac

SEQ ID NO: 372 - DNA, 25 bp, Artificial Sequence
  Description of Artificial Sequence: Synthetic oligonucleotide
ctgtaagataccaaaaaggcaaaac

SEQ ID NO: 373 - DNA, 25 bp, Artificial Sequence
  Description of Artificial Sequence: Synthetic oligonucleotide
cctgtaagataccaaaaaggcaaaa

SEQ ID NO: 374 - DNA, 25 bp, Artificial Sequence
  Description of Artificial Sequence: Synthetic oligonucleotide
agttcctgtaagataccaaaaaggc

SEQ ID NO: 375 - DNA, 25 bp, Artificial Sequence
  Description of Artificial Sequence: Synthetic oligonucleotide
gagttcctgtaagataccaaaaagg

SEQ ID NO: 376 - DNA, 25 bp, Artificial Sequence
  Description of Artificial Sequence: Synthetic oligonucleotide
cctggagttcctgtaagataccaaa

SEQ ID NO: 377 - DNA, 25 bp, Artificial Sequence
  Description of Artificial Sequence: Synthetic oligonucleotide
tcctggagttcctgtaagataccaa

SEQ ID NO: 378 - DNA, 25 bp, Artificial Sequence
  Description of Artificial Sequence: Synthetic oligonucleotide
gccatcctggagttcctgtaagata

SEQ ID NO: 379 - DNA, 25 bp, Artificial Sequence
  Description of Artificial Sequence: Synthetic oligonucleotide
tgccatcctggagttcctgtaagat

SEQ ID NO: 380 - DNA, 25 bp, Artificial Sequence
  Description of Artificial Sequence: Synthetic oligonucleotide
ccaatgccatcctggagttcctgta

SEQ ID NO: 381 - DNA, 25 bp, Artificial Sequence
  Description of Artificial Sequence: Synthetic oligonucleotide
cccaatgccatcctggagttcctgt

SEQ ID NO: 382 - DNA, 25 bp, Artificial Sequence
  Description of Artificial Sequence: Synthetic oligonucleotide
gctgcccaatgccatcctggagttc

SEQ ID NO: 383 - DNA, 25 bp, Artificial Sequence
  Description of Artificial Sequence: Synthetic oligonucleotide
cgctgcccaatgccatcctggagtt

SEQ ID NO: 384 - DNA, 25 bp, Artificial Sequence
  Description of Artificial Sequence: Synthetic oligonucleotide
aacagtttgccgctgcccaatgcca

SEQ ID NO: 385 - DNA, 25 bp, Artificial Sequence
  Description of Artificial Sequence: Synthetic oligonucleotide
ctgacaacagtttgccgctgcccaa

SEQ ID NO: 386 - DNA, 25 bp, Artificial Sequence
  Description of Artificial Sequence: Synthetic oligonucleotide
gttgcattcaatgttctgacaacag

SEQ ID NO: 387 - DNA, 25 bp, Artificial Sequence
  Description of Artificial Sequence: Synthetic oligonucleotide
gctgaattatttcttccccagttgc

SEQ ID NO: 388 - DNA, 25 bp, Artificial Sequence
  Description of Artificial Sequence: Synthetic oligonucleotide
attatttcttccccagttgcattca

SEQ ID NO: 389 - DNA, 25 bp, Artificial Sequence
  Description of Artificial Sequence: Synthetic oligonucleotide
ggcatctgtttttgaggattgctga

SEQ ID NO: 390 - DNA, 25 bp, Artificial Sequence
  Description of Artificial Sequence: Synthetic oligonucleotide
tttgaggattgctgaattatttctt

SEQ ID NO: 391 - DNA, 25 bp, Artificial Sequence
  Description of Artificial Sequence: Synthetic oligonucleotide
aatttttcctgtagaatactggcat

SEQ ID NO: 392 - DNA, 25 bp, Artificial Sequence
  Description of Artificial Sequence: Synthetic oligonucleotide
atactggcatctgtttttgaggatt

SEQ ID NO: 393 - DNA, 25 bp, Artificial Sequence
  Description of Artificial Sequence: Synthetic oligonucleotide
accgcagattcaggcttcccaattt

SEQ ID NO: 394 - DNA, 25 bp, Artificial Sequence
  Description of Artificial Sequence: Synthetic oligonucleotide
aatttttcctgtagaatactggcat

SEQ ID NO: 395 - DNA, 25 bp, Artificial Sequence
  Description of Artificial Sequence: Synthetic oligonucleotide
ctgtttgcagacctcctgccaccgc

SEQ ID NO: 396 - DNA, 25 bp, Artificial Sequence
  Description of Artificial Sequence: Synthetic oligonucleotide
agattcaggcttcccaatttttcct

SEQ ID NO: 397 - DNA, 25 bp, Artificial Sequence
  Description of Artificial Sequence: Synthetic oligonucleotide
ctcttttttctgtctgacagctgtt

SEQ ID NO: 398 - DNA, 25 bp, Artificial Sequence
  Description of Artificial Sequence: Synthetic oligonucleotide
acctcctgccaccgcagattcaggc

SEQ ID NO: 399 - DNA, 25 bp, Artificial Sequence
  Description of Artificial Sequence: Synthetic oligonucleotide
cctacctcttttttctgtctgacag

SEQ ID NO: 400 - DNA, 25 bp, Artificial Sequence
  Description of Artificial Sequence: Synthetic oligonucleotide
gacagctgtttgcagacctcctgcc

SEQ ID NO: 401 - DNA, 25 bp, Artificial Sequence
  Description of Artificial Sequence: Synthetic oligonucleotide
gtcgccctacctcttttttctgtct

SEQ ID NO: 402 - DNA, 25 bp, Artificial Sequence
  Description of Artificial Sequence: Synthetic oligonucleotide
gatctgtcgccctacctcttttttc

SEQ ID NO: 403 - DNA, 25 bp, Artificial Sequence
  Description of Artificial Sequence: Synthetic oligonucleotide
tattagatctgtcgccctacctctt

SEQ ID NO: 404 - DNA, 25 bp, Artificial Sequence
  Description of Artificial Sequence: Synthetic oligonucleotide
attcctattagatctgtcgccctac

SEQ ID NO: 405 - DNA, 20 bp, Artificial Sequence
  Description of Artificial Sequence: Synthetic oligonucleotide
agataccaaaaaggcaaaac

SEQ ID NO: 406 - DNA, 20 bp, Artificial Sequence
  Description of Artificial Sequence: Synthetic oligonucleotide
aagataccaaaaaggcaaaa

SEQ ID NO: 407 - DNA, 20 bp, Artificial Sequence
  Description of Artificial Sequence: Synthetic oligonucleotide
cctgtaagataccaaaaagg

SEQ ID NO: 408 - DNA, 20 bp, Artificial Sequence
  Description of Artificial Sequence: Synthetic oligonucleotide
gagttcctgtaagataccaa

SEQ ID NO: 409 - DNA, 20 bp, Artificial Sequence
  Description of Artificial Sequence: Synthetic oligonucleotide
tcctggagttcctgtaagat

SEQ ID NO: 410 - DNA, 20 bp, Artificial Sequence
  Description of Artificial Sequence: Synthetic oligonucleotide
tgccatcctggagttcctgt

SEQ ID NO: 411 - DNA, 20 bp, Artificial Sequence
  Description of Artificial Sequence: Synthetic oligonucleotide
cccaatgccatcctggagtt

SEQ ID NO: 412 - DNA, 20 bp, Artificial Sequence
  Description of Artificial Sequence: Synthetic oligonucleotide
cgctgcccaatgccatcctg

SEQ ID NO: 413 - DNA, 20 bp, Artificial Sequence
  Description of Artificial Sequence: Synthetic oligonucleotide
ctgacaacagtttgccgctg

SEQ ID NO: 414 - DNA, 20 bp, Artificial Sequence
  Description of Artificial Sequence: Synthetic oligonucleotide
gttgcattcaatgttctgac

SEQ ID NO: 415 - DNA, 20 bp, Artificial Sequence
  Description of Artificial Sequence: Synthetic oligonucleotide
attatttcttccccagttgc

SEQ ID NO: 416 - DNA, 20 bp, Artificial Sequence
  Description of Artificial Sequence: Synthetic oligonucleotide
tttgaggattgctgaattat

SEQ ID NO: 417 - DNA, 20 bp, Artificial Sequence
  Description of Artificial Sequence: Synthetic oligonucleotide
atactggcatctgtttttga

SEQ ID NO: 418 - DNA, 20 bp, Artificial Sequence
  Description of Artificial Sequence: Synthetic oligonucleotide
aatttttcctgtagaatact

SEQ ID NO: 419 - DNA, 20 bp, Artificial Sequence
  Description of Artificial Sequence: Synthetic oligonucleotide
agattcaggcttcccaattt

SEQ ID NO: 420 - DNA, 20 bp, Artificial Sequence
  Description of Artificial Sequence: Synthetic oligonucleotide
acctcctgccaccgcagatt

SEQ ID NO: 421 - DNA, 20 bp, Artificial Sequence
  Description of Artificial Sequence: Synthetic oligonucleotide
gacagctgtttgcagacctc

SEQ ID NO: 422 - DNA, 20 bp, Artificial Sequence
  Description of Artificial Sequence: Synthetic oligonucleotide
ctcttttttctgtctgacag

SEQ ID NO: 423 - DNA, 20 bp, Artificial Sequence
  Description of Artificial Sequence: Synthetic oligonucleotide
cctacctcttttttctgtct

SEQ ID NO: 424 - DNA, 20 bp, Artificial Sequence
  Description of Artificial Sequence: Synthetic oligonucleotide
gtcgccctacctcttttttc

SEQ ID NO: 425 - DNA, 20 bp, Artificial Sequence
  Description of Artificial Sequence: Synthetic oligonucleotide
gatctgtcgccctacctctt

SEQ ID NO: 426 - DNA, 20 bp, Artificial Sequence
  Description of Artificial Sequence: Synthetic oligonucleotide
tattagatctgtcgccctac

SEQ ID NO: 427 - DNA, 20 bp, Artificial Sequence
  Description of Artificial Sequence: Synthetic oligonucleotide
attcctattagatctgtcgc

SEQ ID NO: 428 - DNA, 25 bp, Artificial Sequence
  Description of Artificial Sequence: Synthetic oligonucleotide
gggggatttgagaaaataaaattac

SEQ ID NO: 429 - DNA, 25 bp, Artificial Sequence
  Description of Artificial Sequence: Synthetic oligonucleotide
atttgagaaaataaaattaccttga

SEQ ID NO: 430 - DNA, 25 bp, Artificial Sequence
  Description of Artificial Sequence: Synthetic oligonucleotide
ctagcctggagaaagaagaataaaa

SEQ ID NO: 431 - DNA, 25 bp, Artificial Sequence
  Description of Artificial Sequence: Synthetic oligonucleotide
agaaaataaaattaccttgacttgc

SEQ ID NO: 432 - DNA, 25 bp, Artificial Sequence
  Description of Artificial Sequence: Synthetic oligonucleotide
ttcttctagcctggagaaagaagaa

SEQ ID NO: 433 - DNA, 25 bp, Artificial Sequence
  Description of Artificial Sequence: Synthetic oligonucleotide
ataaaattaccttgacttgctcaag

SEQ ID NO: 434 - DNA, 25 bp, Artificial Sequence
  Description of Artificial Sequence: Synthetic oligonucleotide
ttttgttcttctagcctggagaaag

SEQ ID NO: 435 - DNA, 25 bp, Artificial Sequence
  Description of Artificial Sequence: Synthetic oligonucleotide
attaccttgacttgctcaagctttt

SEQ ID NO: 436 - DNA, 25 bp, Artificial Sequence
  Description of Artificial Sequence: Synthetic oligonucleotide
tattcttttgttcttctagcctgga

SEQ ID NO: 437 - DNA, 25 bp, Artificial Sequence
  Description of Artificial Sequence: Synthetic oligonucleotide
cttgacttgctcaagcttttctttt

SEQ ID NO: 438 - DNA, 25 bp, Artificial Sequence
  Description of Artificial Sequence: Synthetic oligonucleotide
caagatattcttttgttcttctagc

SEQ ID NO: 439 - DNA, 25 bp, Artificial Sequence
  Description of Artificial Sequence: Synthetic oligonucleotide
cttttagttgctgctcttttccagg

SEQ ID NO: 440 - DNA, 25 bp, Artificial Sequence
  Description of Artificial Sequence: Synthetic oligonucleotide
ccaggttcaagtgggatactagcaa

SEQ ID NO: 441 - DNA, 25 bp, Artificial Sequence
  Description of Artificial Sequence: Synthetic oligonucleotide
atctctttgaaattctgacaagata

SEQ ID NO: 442 - DNA, 25 bp, Artificial Sequence
  Description of Artificial Sequence: Synthetic oligonucleotide
agcaatgttatctgcttcctccaac

SEQ ID NO: 443 - DNA, 25 bp, Artificial Sequence
  Description of Artificial Sequence: Synthetic oligonucleotide
aacaaattcatttaaatctctttga

SEQ ID NO: 444 - DNA, 25 bp, Artificial Sequence
  Description of Artificial Sequence: Synthetic oligonucleotide
ccaaccataaaacaaattcatttaa

SEQ ID NO: 445 - DNA, 25 bp, Artificial Sequence
  Description of Artificial Sequence: Synthetic oligonucleotide
ttcctccaaccataaaacaaattca

SEQ ID NO: 446 - DNA, 25 bp, Artificial Sequence
  Description of Artificial Sequence: Synthetic oligonucleotide
tttaaatctctttgaaattctgaca

SEQ ID NO: 447 - DNA, 25 bp, Artificial Sequence
  Description of Artificial Sequence: Synthetic oligonucleotide
tgacaagatattcttttgttcttct

SEQ ID NO: 448 - DNA, 25 bp, Artificial Sequence
  Description of Artificial Sequence: Synthetic oligonucleotide
ttcaagtgggatactagcaatgtta

SEQ ID NO: 449 - DNA, 25 bp, Artificial Sequence
  Description of Artificial Sequence: Synthetic oligonucleotide
agatattcttttgttcttctagcct

SEQ ID NO: 450 - DNA, 25 bp, Artificial Sequence
  Description of Artificial Sequence: Synthetic oligonucleotide
ctgctcttttccaggttcaagtggg

SEQ ID NO: 451 - DNA, 25 bp, Artificial Sequence
  Description of Artificial Sequence: Synthetic oligonucleotide
ttcttttgttcttctagcctggaga

SEQ ID NO: 452 - DNA, 25 bp, Artificial Sequence
  Description of Artificial Sequence: Synthetic oligonucleotide
cttttcttttagttgctgctctttt

SEQ ID NO: 453 - DNA, 25 bp, Artificial Sequence
  Description of Artificial Sequence: Synthetic oligonucleotide
ttgttcttctagcctggagaaagaa

SEQ ID NO: 454 - DNA, 25 bp, Artificial Sequence
  Description of Artificial Sequence: Synthetic oligonucleotide
cttctagcctggagaaagaagaata

SEQ ID NO: 455 - DNA, 25 bp, Artificial Sequence
  Description of Artificial Sequence: Synthetic oligonucleotide
agcctggagaaagaagaataaaatt

SEQ ID NO: 456 - DNA, 25 bp, Artificial Sequence
  Description of Artificial Sequence: Synthetic oligonucleotide
ctggagaaagaagaataaaattgtt

SEQ ID NO: 457 - DNA, 20 bp, Artificial Sequence
  Description of Artificial Sequence: Synthetic oligonucleotide
gaaagaagaataaaattgtt

SEQ ID NO: 458 - DNA, 20 bp, Artificial Sequence
  Description of Artificial Sequence: Synthetic oligonucleotide
ggagaaagaagaataaaatt

SEQ ID NO: 459 - DNA, 20 bp, Artificial Sequence
  Description of Artificial Sequence: Synthetic oligonucleotide
agcctggagaaagaagaata

SEQ ID NO: 460 - DNA, 20 bp, Artificial Sequence
  Description of Artificial Sequence: Synthetic oligonucleotide
cttctagcctggagaaagaa

SEQ ID NO: 461 - DNA, 20 bp, Artificial Sequence
  Description of Artificial Sequence: Synthetic oligonucleotide
ttgttcttctagcctggaga

SEQ ID NO: 462 - DNA, 20 bp, Artificial Sequence
  Description of Artificial Sequence: Synthetic oligonucleotide
ttcttttgttcttctagcct

SEQ ID NO: 463 - DNA, 20 bp, Artificial Sequence
  Description of Artificial Sequence: Synthetic oligonucleotide
tgacaagatattcttttgtt

SEQ ID NO: 464 - DNA, 20 bp, Artificial Sequence
  Description of Artificial Sequence: Synthetic oligonucleotide
atctctttgaaattctgaca

SEQ ID NO: 465 - DNA, 20 bp, Artificial Sequence
  Description of Artificial Sequence: Synthetic oligonucleotide
aacaaattcatttaaatctc

SEQ ID NO: 466 - DNA, 20 bp, Artificial Sequence
  Description of Artificial Sequence: Synthetic oligonucleotide
ttcctccaaccataaaacaa

SEQ ID NO: 467 - DNA, 20 bp, Artificial Sequence
  Description of Artificial Sequence: Synthetic oligonucleotide
agcaatgttatctgcttcct

SEQ ID NO: 468 - DNA, 20 bp, Artificial Sequence
  Description of Artificial Sequence: Synthetic oligonucleotide
ttcaagtgggatactagcaa

SEQ ID NO: 469 - DNA, 20 bp, Artificial Sequence
  Description of Artificial Sequence: Synthetic oligonucleotide
ctgctcttttccaggttcaa

SEQ ID NO: 470 - DNA, 20 bp, Artificial Sequence
  Description of Artificial Sequence: Synthetic oligonucleotide
cttttcttttagttgctgct

SEQ ID NO: 471 - DNA, 20 bp, Artificial Sequence
  Description of Artificial Sequence: Synthetic oligonucleotide
cttgacttgctcaagctttt

SEQ ID NO: 472 - DNA, 20 bp, Artificial Sequence
  Description of Artificial Sequence: Synthetic oligonucleotide
attaccttgacttgctcaag

SEQ ID NO: 473 - DNA, 20 bp, Artificial Sequence
  Description of Artificial Sequence: Synthetic oligonucleotide
ataaaattaccttgacttgc

SEQ ID NO: 474 - DNA, 20 bp, Artificial Sequence
  Description of Artificial Sequence: Synthetic oligonucleotide
agaaaataaaattaccttga

SEQ ID NO: 475 - DNA, 20 bp, Artificial Sequence
  Description of Artificial Sequence: Synthetic oligonucleotide
atttgagaaaataaaattac

SEQ ID NO: 476 - DNA, 20 bp, Artificial Sequence
  Description of Artificial Sequence: Synthetic oligonucleotide
gggggatttgagaaaataaa

SEQ ID NO: 477 - DNA, 25 bp, Artificial Sequence
  Description of Artificial Sequence: Synthetic oligonucleotide
ctgaaacagacaaatgcaacaacgt

SEQ ID NO: 478 - DNA, 25 bp, Artificial Sequence
  Description of Artificial Sequence: Synthetic oligonucleotide
agtaactgaaacagacaaatgcaac

SEQ ID NO: 479 - DNA, 25 bp, Artificial Sequence
  Description of Artificial Sequence: Synthetic oligonucleotide
ccaccagtaactgaaacagacaaat

SEQ ID NO: 480 - DNA, 25 bp, Artificial Sequence
  Description of Artificial Sequence: Synthetic oligonucleotide
ctcttccaccagtaactgaaacaga

SEQ ID NO: 481 - DNA, 25 bp, Artificial Sequence
  Description of Artificial Sequence: Synthetic oligonucleotide
ggcaactcttccaccagtaactgaa

SEQ ID NO: 482 - DNA, 25 bp, Artificial Sequence
  Description of Artificial Sequence: Synthetic oligonucleotide
gcaggggcaactcttccaccagtaa

SEQ ID NO: 483 - DNA, 25 bp, Artificial Sequence
  Description of Artificial Sequence: Synthetic oligonucleotide
ctggcgcaggggcaactcttccacc

SEQ ID NO: 484 - DNA, 25 bp, Artificial Sequence
  Description of Artificial Sequence: Synthetic oligonucleotide
tttaattgtttgagaattccctggc

SEQ ID NO: 485 - DNA, 25 bp, Artificial Sequence
  Description of Artificial Sequence: Synthetic oligonucleotide
ttgtttgagaattccctggcgcagg

SEQ ID NO: 486 - DNA, 25 bp, Artificial Sequence
  Description of Artificial Sequence: Synthetic oligonucleotide
gcacgggtcctccagtttcatttaa

SEQ ID NO: 487 - DNA, 25 bp, Artificial Sequence
  Description of Artificial Sequence: Synthetic oligonucleotide
tccagtttcatttaattgtttgaga

SEQ ID NO: 488 - DNA, 25 bp, Artificial Sequence
  Description of Artificial Sequence: Synthetic oligonucleotide
gcttatgggagcacttacaagcacg

SEQ ID NO: 489 - DNA, 25 bp, Artificial Sequence
  Description of Artificial Sequence: Synthetic oligonucleotide
tacaagcacgggtcctccagtttca

SEQ ID NO: 490 - DNA, 25 bp, Artificial Sequence
  Description of Artificial Sequence: Synthetic oligonucleotide
agtttatcttgctcttctgggctta

SEQ ID NO: 491 - DNA, 25 bp, Artificial Sequence
  Description of Artificial Sequence: Synthetic oligonucleotide
tctgcttgagcttattttcaagttt

SEQ ID NO: 492 - DNA, 25 bp, Artificial Sequence
  Description of Artificial Sequence: Synthetic oligonucleotide
atcttgctcttctgggcttatggga

SEQ ID NO: 493 - DNA, 25 bp, Artificial Sequence
  Description of Artificial Sequence: Synthetic oligonucleotide
ctttatccactggagatttgtctgc

SEQ ID NO: 494 - DNA, 25 bp, Artificial Sequence
  Description of Artificial Sequence: Synthetic oligonucleotide
cttattttcaagtttatcttgctct

SEQ ID NO: 495 - DNA, 25 bp, Artificial Sequence
  Description of Artificial Sequence: Synthetic oligonucleotide
ctaacctttatccactggagatttg

SEQ ID NO: 496 - DNA, 25 bp, Artificial Sequence
  Description of Artificial Sequence: Synthetic oligonucleotide
atttgtctgcttgagcttattttca

SEQ ID NO: 497 - DNA, 25 bp, Artificial Sequence
  Description of Artificial Sequence: Synthetic oligonucleotide
aatgtctaacctttatccactggag

SEQ ID NO: 498 - DNA, 25 bp, Artificial Sequence
  Description of Artificial Sequence: Synthetic oligonucleotide
tggttaatgtctaacctttatccac

SEQ ID NO: 499 - DNA, 25 bp, Artificial Sequence
  Description of Artificial Sequence: Synthetic oligonucleotide
agagatggttaatgtctaaccttta

SEQ ID NO: 500 - DNA, 25 bp, Artificial Sequence
  Description of Artificial Sequence: Synthetic oligonucleotide
acggaagagatggttaatgtctaac

SEQ ID NO: 501 - DNA, 20 bp, Artificial Sequence
  Description of Artificial Sequence: Synthetic oligonucleotide
acagacaaatgcaacaacgt

SEQ ID NO: 502 - DNA, 20 bp, Artificial Sequence
  Description of Artificial Sequence: Synthetic oligonucleotide
ctgaaacagacaaatgcaac

SEQ ID NO: 503 - DNA, 20 bp, Artificial Sequence
  Description of Artificial Sequence: Synthetic oligonucleotide
agtaactgaaacagacaaat

SEQ ID NO: 504 - DNA, 20 bp, Artificial Sequence
  Description of Artificial Sequence: Synthetic oligonucleotide
ccaccagtaactgaaacaga

SEQ ID NO: 505 - DNA, 20 bp, Artificial Sequence
  Description of Artificial Sequence: Synthetic oligonucleotide
ctcttccaccagtaactgaa

SEQ ID NO: 506 - DNA, 20 bp, Artificial Sequence
  Description of Artificial Sequence: Synthetic oligonucleotide
ggcaactcttccaccagtaa

SEQ ID NO: 507 - DNA, 20 bp, Artificial Sequence
  Description of Artificial Sequence: Synthetic oligonucleotide
ctggcgcaggggcaactctt

SEQ ID NO: 508 - DNA, 20 bp, Artificial Sequence
  Description of Artificial Sequence: Synthetic oligonucleotide
ttgtttgagaattccctggc

SEQ ID NO: 509 - DNA, 20 bp, Artificial Sequence
  Description of Artificial Sequence: Synthetic oligonucleotide
tccagtttcatttaattgtt

SEQ ID NO: 510 - DNA, 20 bp, Artificial Sequence
  Description of Artificial Sequence: Synthetic oligonucleotide
tacaagcacgggtcctccag

SEQ ID NO: 511 - DNA, 20 bp, Artificial Sequence
  Description of Artificial Sequence: Synthetic oligonucleotide
gcttatgggagcacttacaa

SEQ ID NO: 512 - DNA, 20 bp, Artificial Sequence
  Description of Artificial Sequence: Synthetic oligonucleotide
atcttgctcttctgggctta

SEQ ID NO: 513 - DNA, 20 bp, Artificial Sequence
  Description of Artificial Sequence: Synthetic oligonucleotide
cttattttcaagtttatctt

SEQ ID NO: 514 - DNA, 20 bp, Artificial Sequence
  Description of Artificial Sequence: Synthetic oligonucleotide
atttgtctgcttgagcttat

SEQ ID NO: 515 - DNA, 20 bp, Artificial Sequence
  Description of Artificial Sequence: Synthetic oligonucleotide
ctttatccactggagatttg

SEQ ID NO: 516 - DNA, 20 bp, Artificial Sequence
  Description of Artificial Sequence: Synthetic oligonucleotide
ctaacctttatccactggag

SEQ ID NO: 517 - DNA, 20 bp, Artificial Sequence
  Description of Artificial Sequence: Synthetic oligonucleotide
aatgtctaacctttatccac

SEQ ID NO: 518 - DNA, 20 bp, Artificial Sequence
  Description of Artificial Sequence: Synthetic oligonucleotide
tggttaatgtctaaccttta

SEQ ID NO: 519 - DNA, 20 bp, Artificial Sequence
  Description of Artificial Sequence: Synthetic oligonucleotide
agagatggttaatgtctaac

SEQ ID NO: 520 - DNA, 20 bp, Artificial Sequence
  Description of Artificial Sequence: Synthetic oligonucleotide
acggaagagatggttaatgt

SEQ ID NO: 521 - DNA, 25 bp, Artificial Sequence
  Description of Artificial Sequence: Synthetic oligonucleotide
ctgaaaggaaaatacattttaaaaa

SEQ ID NO: 522 - DNA, 25 bp, Artificial Sequence
  Description of Artificial Sequence: Synthetic oligonucleotide
cctgaaaggaaaatacattttaaaa

SEQ ID NO: 523 - DNA, 25 bp, Artificial Sequence
  Description of Artificial Sequence: Synthetic oligonucleotide
gaaacctgaaaggaaaatacatttt

SEQ ID NO: 524 - DNA, 25 bp, Artificial Sequence
  Description of Artificial Sequence: Synthetic oligonucleotide
ggaaacctgaaaggaaaatacattt

SEQ ID NO: 525 - DNA, 25 bp, Artificial Sequence
  Description of Artificial Sequence: Synthetic oligonucleotide
ctctggaaacctgaaaggaaaatac

SEQ ID NO: 526 - DNA, 25 bp, Artificial Sequence
  Description of Artificial Sequence: Synthetic oligonucleotide
gctctggaaacctgaaaggaaaata

SEQ ID NO: 527 - DNA, 25 bp, Artificial Sequence
  Description of Artificial Sequence: Synthetic oligonucleotide
taaagctctggaaacctgaaaggaa

SEQ ID NO: 528 - DNA, 25 bp, Artificial Sequence
  Description of Artificial Sequence: Synthetic oligonucleotide
gtaaagctctggaaacctgaaagga

SEQ ID NO: 529 - DNA, 25 bp, Artificial Sequence
  Description of Artificial Sequence: Synthetic oligonucleotide
tcaggtaaagctctggaaacctgaa

SEQ ID NO: 530 - DNA, 25 bp, Artificial Sequence
  Description of Artificial Sequence: Synthetic oligonucleotide
ctcaggtaaagctctggaaacctga

SEQ ID NO: 531 - DNA, 25 bp, Artificial Sequence
  Description of Artificial Sequence: Synthetic oligonucleotide
gtttctcaggtaaagctctggaaac

SEQ ID NO: 532 - DNA, 25 bp, Artificial Sequence
  Description of Artificial Sequence: Synthetic oligonucleotide
tgtttctcaggtaaagctctggaaa

SEQ ID NO: 533 - DNA, 25 bp, Artificial Sequence
  Description of Artificial Sequence: Synthetic oligonucleotide
aatttctccttgtttctcaggtaaa

SEQ ID NO: 534 - DNA, 25 bp, Artificial Sequence
  Description of Artificial Sequence: Synthetic oligonucleotide
tttgagcttcaatttctccttgttt

SEQ ID NO: 535 - DNA, 25 bp, Artificial Sequence
  Description of Artificial Sequence: Synthetic oligonucleotide
ttttatttgagcttcaatttctcct

SEQ ID NO: 536 - DNA, 25 bp, Artificial Sequence
  Description of Artificial Sequence: Synthetic oligonucleotide
aagctgcccaaggtcttttatttga

SEQ ID NO: 537 - DNA, 25 bp, Artificial Sequence
  Description of Artificial Sequence: Synthetic oligonucleotide
aggtcttcaagctttttttcaagct

SEQ ID NO: 538 - DNA, 25 bp, Artificial Sequence
  Description of Artificial Sequence: Synthetic oligonucleotide
ttcaagctttttttcaagctgccca

SEQ ID NO: 539 - DNA, 25 bp, Artificial Sequence
  Description of Artificial Sequence: Synthetic oligonucleotide
gatgatttaactgctcttcaaggtc

SEQ ID NO: 540 - DNA, 25 bp, Artificial Sequence
  Description of Artificial Sequence: Synthetic oligonucleotide
ctgctcttcaaggtcttcaagcttt

SEQ ID NO: 541 - DNA, 25 bp, Artificial Sequence
  Description of Artificial Sequence: Synthetic oligonucleotide
aggagataaccacagcagcagatga

SEQ ID NO: 542 - DNA, 25 bp, Artificial Sequence
  Description of Artificial Sequence: Synthetic oligonucleotide
cagcagatgatttaactgctcttca

SEQ ID NO: 543 - DNA, 25 bp, Artificial Sequence
  Description of Artificial Sequence: Synthetic oligonucleotide
atttccaactgattcctaataggag

SEQ ID NO: 544 - DNA, 25 bp, Artificial Sequence
  Description of Artificial Sequence: Synthetic oligonucleotide
cttggtttggttggttataaatttc

SEQ ID NO: 545 - DNA, 25 bp, Artificial Sequence
  Description of Artificial Sequence: Synthetic oligonucleotide
caactgattcctaataggagataac

SEQ ID NO: 546 - DNA, 25 bp, Artificial Sequence
  Description of Artificial Sequence: Synthetic oligonucleotide
cttaacgtcaaatggtccttcttgg

SEQ ID NO: 547 - DNA, 25 bp, Artificial Sequence
  Description of Artificial Sequence: Synthetic oligonucleotide
ttggttataaatttccaactgattc

SEQ ID NO: 548 - DNA, 25 bp, Artificial Sequence
  Description of Artificial Sequence: Synthetic oligonucleotide
cctaccttaacgtcaaatggtcctt

SEQ ID NO: 549 - DNA, 25 bp, Artificial Sequence
  Description of Artificial Sequence: Synthetic oligonucleotide
tccttcttggtttggttggttataa

SEQ ID NO: 550 - DNA, 25 bp, Artificial Sequence
  Description of Artificial Sequence: Synthetic oligonucleotide
agttccctaccttaacgtcaaatgg

SEQ ID NO: 551 - DNA, 25 bp, Artificial Sequence
  Description of Artificial Sequence: Synthetic oligonucleotide
caaaaagttccctaccttaacgtca

SEQ ID NO: 552 - DNA, 25 bp, Artificial Sequence
  Description of Artificial Sequence: Synthetic oligonucleotide
taaagcaaaaagttccctaccttaa

SEQ ID NO: 553 - DNA, 25 bp, Artificial Sequence
  Description of Artificial Sequence: Synthetic oligonucleotide
atatttaaagcaaaaagttccctac

SEQ ID NO: 554 - DNA, 20 bp, Artificial Sequence
  Description of Artificial Sequence: Synthetic oligonucleotide
aggaaaatacattttaaaaa

SEQ ID NO: 555 - DNA, 20 bp, Artificial Sequence
  Description of Artificial Sequence: Synthetic oligonucleotide
aaggaaaatacattttaaaa

SEQ ID NO: 556 - DNA, 20 bp, Artificial Sequence
  Description of Artificial Sequence: Synthetic oligonucleotide
cctgaaaggaaaatacattt

SEQ ID NO: 557 - DNA, 20 bp, Artificial Sequence
  Description of Artificial Sequence: Synthetic oligonucleotide
ggaaacctgaaaggaaaata

SEQ ID NO: 558 - DNA, 20 bp, Artificial Sequence
  Description of Artificial Sequence: Synthetic oligonucleotide
gctctggaaacctgaaagga

SEQ ID NO: 559 - DNA, 20 bp, Artificial Sequence
  Description of Artificial Sequence: Synthetic oligonucleotide
gtaaagctctggaaacctga

SEQ ID NO: 560 - DNA, 20 bp, Artificial Sequence
  Description of Artificial Sequence: Synthetic oligonucleotide
ctcaggtaaagctctggaaa

SEQ ID NO: 561 - DNA, 20 bp, Artificial Sequence
  Description of Artificial Sequence: Synthetic oligonucleotide
aatttctccttgtttctcag

SEQ ID NO: 562 - DNA, 20 bp, Artificial Sequence
  Description of Artificial Sequence: Synthetic oligonucleotide
ttttatttgagcttcaattt

SEQ ID NO: 563 - DNA, 20 bp, Artificial Sequence
  Description of Artificial Sequence: Synthetic oligonucleotide
aagctgcccaaggtctttta

SEQ ID NO: 564 - DNA, 20 bp, Artificial Sequence
  Description of Artificial Sequence: Synthetic oligonucleotide
ttcaagctttttttcaagct

SEQ ID NO: 565 - DNA, 20 bp, Artificial Sequence
  Description of Artificial Sequence: Synthetic oligonucleotide
ctgctcttcaaggtcttcaa

SEQ ID NO: 566 - DNA, 20 bp, Artificial Sequence
  Description of Artificial Sequence: Synthetic oligonucleotide
cagcagatgatttaactgct

SEQ ID NO: 567 - DNA, 20 bp, Artificial Sequence
  Description of Artificial Sequence: Synthetic oligonucleotide
aggagataaccacagcagca

SEQ ID NO: 568 - DNA, 20 bp, Artificial Sequence
  Description of Artificial Sequence: Synthetic oligonucleotide
caactgattcctaataggag

SEQ ID NO: 569 - DNA, 20 bp, Artificial Sequence
  Description of Artificial Sequence: Synthetic oligonucleotide
ttggttataaatttccaact

SEQ ID NO: 570 - DNA, 20 bp, Artificial Sequence
  Description of Artificial Sequence: Synthetic oligonucleotide
tccttcttggtttggttggt

SEQ ID NO: 571 - DNA, 20 bp, Artificial Sequence
  Description of Artificial Sequence: Synthetic oligonucleotide
cttaacgtcaaatggtcctt

SEQ ID NO: 572 - DNA, 20 bp, Artificial Sequence
  Description of Artificial Sequence: Synthetic oligonucleotide
cctaccttaacgtcaaatgg

SEQ ID NO: 573 - DNA, 20 bp, Artificial Sequence
  Description of Artificial Sequence: Synthetic oligonucleotide
agttccctaccttaacgtca

SEQ ID NO: 574 - DNA, 20 bp, Artificial Sequence
  Description of Artificial Sequence: Synthetic oligonucleotide
caaaaagttccctaccttaa

SEQ ID NO: 575 - DNA, 20 bp, Artificial Sequence
  Description of Artificial Sequence: Synthetic oligonucleotide
taaagcaaaaagttccctac

SEQ ID NO: 576 - DNA, 20 bp, Artificial Sequence
  Description of Artificial Sequence: Synthetic oligonucleotide
atatttaaagcaaaaagttc

SEQ ID NO: 577 - DNA, 25 bp, Artificial Sequence
  Description of Artificial Sequence: Synthetic oligonucleotide
ctggggaaaagaacccatatagtgc

SEQ ID NO: 578 - DNA, 25 bp, Artificial Sequence
  Description of Artificial Sequence: Synthetic oligonucleotide
tcctggggaaaagaacccatatagt

SEQ ID NO: 579 - DNA, 25 bp, Artificial Sequence
  Description of Artificial Sequence: Synthetic oligonucleotide
gtttcctggggaaaagaacccatat

SEQ ID NO: 580 - DNA, 25 bp, Artificial Sequence
  Description of Artificial Sequence: Synthetic oligonucleotide
cagtttcctggggaaaagaacccat

SEQ ID NO: 581 - DNA, 25 bp, Artificial Sequence
  Description of Artificial Sequence: Synthetic oligonucleotide
tttcagtttcctggggaaaagaacc

SEQ ID NO: 582 - DNA, 25 bp, Artificial Sequence
  Description of Artificial Sequence: Synthetic oligonucleotide
tatttcagtttcctggggaaaagaa

SEQ ID NO: 583 - DNA, 25 bp, Artificial Sequence
  Description of Artificial Sequence: Synthetic oligonucleotide
tgctatttcagtttcctggggaaaa

SEQ ID NO: 584 - DNA, 25 bp, Artificial Sequence
  Description of Artificial Sequence: Synthetic oligonucleotide
actgctatttcagtttcctggggaa

SEQ ID NO: 585 - DNA, 25 bp, Artificial Sequence
  Description of Artificial Sequence: Synthetic oligonucleotide
tgaactgctatttcagtttcctggg

SEQ ID NO: 586 - DNA, 25 bp, Artificial Sequence
  Description of Artificial Sequence: Synthetic oligonucleotide
cttgaactgctatttcagtttcctg

SEQ ID NO: 587 - DNA, 25 bp, Artificial Sequence
  Description of Artificial Sequence: Synthetic oligonucleotide
tagcttgaactgctatttcagtttc

SEQ ID NO: 588 - DNA, 25 bp, Artificial Sequence
  Description of Artificial Sequence: Synthetic oligonucleotide
tttagcttgaactgctatttcagtt

SEQ ID NO: 589 - DNA, 25 bp, Artificial Sequence
  Description of Artificial Sequence: Synthetic oligonucleotide
ttccacatccggttgtttagcttga

SEQ ID NO: 590 - DNA, 25 bp, Artificial Sequence
  Description of Artificial Sequence: Synthetic oligonucleotide
tgccctttagacaaaatctcttcca

SEQ ID NO: 591 - DNA, 25 bp, Artificial Sequence
  Description of Artificial Sequence: Synthetic oligonucleotide
tttagacaaaatctcttccacatcc

SEQ ID NO: 592 - DNA, 25 bp, Artificial Sequence
  Description of Artificial Sequence: Synthetic oligonucleotide
gtttttccttgtacaaatgctgccc

SEQ ID NO: 593 - DNA, 25 bp, Artificial Sequence
  Description of Artificial Sequence: Synthetic oligonucleotide
gtacaaatgctgccctttagacaaa

SEQ ID NO: 594 - DNA, 25 bp, Artificial Sequence
  Description of Artificial Sequence: Synthetic oligonucleotide
cttcactggctgagtggctggtttt

SEQ ID NO: 595 - DNA, 25 bp, Artificial Sequence
  Description of Artificial Sequence: Synthetic oligonucleotide
ggctggtttttccttgtacaaatgc

SEQ ID NO: 596 - DNA, 25 bp, Artificial Sequence
  Description of Artificial Sequence: Synthetic oligonucleotide
attaccttcactggctgagtggctg

SEQ ID NO: 597 - DNA, 25 bp, Artificial Sequence
  Description of Artificial Sequence: Synthetic oligonucleotide
gcttcattaccttcactggctgagt

SEQ ID NO: 598 - DNA, 25 bp, Artificial Sequence
  Description of Artificial Sequence: Synthetic oligonucleotide
aggttgcttcattaccttcactggc

SEQ ID NO: 599 - DNA, 25 bp, Artificial Sequence
  Description of Artificial Sequence: Synthetic oligonucleotide
gctagaggttgcttcattaccttca

SEQ ID NO: 600 - DNA, 25 bp, Artificial Sequence
  Description of Artificial Sequence: Synthetic oligonucleotide
atattgctagaggttgcttcattac

SEQ ID NO: 601 - DNA, 20 bp, Artificial Sequence
  Description of Artificial Sequence: Synthetic oligonucleotide
gaaaagaacccatatagtgc

SEQ ID NO: 602 - DNA, 20 bp, Artificial Sequence
  Description of Artificial Sequence: Synthetic oligonucleotide
gggaaaagaacccatatagt

SEQ ID NO: 603 - DNA, 20 bp, Artificial Sequence
  Description of Artificial Sequence: Synthetic oligonucleotide
tcctggggaaaagaacccat

SEQ ID NO: 604 - DNA, 20 bp, Artificial Sequence
  Description of Artificial Sequence: Synthetic oligonucleotide
cagtttcctggggaaaagaa

SEQ ID NO: 605 - DNA, 20 bp, Artificial Sequence
  Description of Artificial Sequence: Synthetic oligonucleotide
tatttcagtttcctggggaa

SEQ ID NO: 606 - DNA, 20 bp, Artificial Sequence
  Description of Artificial Sequence: Synthetic oligonucleotide
actgctatttcagtttcctg

SEQ ID NO: 607 - DNA, 20 bp, Artificial Sequence
  Description of Artificial Sequence: Synthetic oligonucleotide
cttgaactgctatttcagtt

SEQ ID NO: 608 - DNA, 20 bp, Artificial Sequence
  Description of Artificial Sequence: Synthetic oligonucleotide
tttagcttgaactgctattt

SEQ ID NO: 609 - DNA, 20 bp, Artificial Sequence
  Description of Artificial Sequence: Synthetic oligonucleotide
ttccacatccggttgtttag

SEQ ID NO: 610 - DNA, 20 bp, Artificial Sequence
  Description of Artificial Sequence: Synthetic oligonucleotide
tttagacaaaatctcttcca

SEQ ID NO: 611 - DNA, 20 bp, Artificial Sequence
  Description of Artificial Sequence: Synthetic oligonucleotide
gtacaaatgctgccctttag

SEQ ID NO: 612 - DNA, 20 bp, Artificial Sequence
  Description of Artificial Sequence: Synthetic oligonucleotide
ggctggtttttccttgtaca

SEQ ID NO: 613 - DNA, 20 bp, Artificial Sequence
  Description of Artificial Sequence: Synthetic oligonucleotide
cttcactggctgagtggctg

SEQ ID NO: 614 - DNA, 20 bp, Artificial Sequence
  Description of Artificial Sequence: Synthetic oligonucleotide
attaccttcactggctgagt

SEQ ID NO: 615 - DNA, 20 bp, Artificial Sequence
  Description of Artificial Sequence: Synthetic oligonucleotide
gcttcattaccttcactggc

SEQ ID NO: 616 - DNA, 20 bp, Artificial Sequence
  Description of Artificial Sequence: Synthetic oligonucleotide
aggttgcttcattaccttca

SEQ ID NO: 617 - DNA, 20 bp, Artificial Sequence
  Description of Artificial Sequence: Synthetic oligonucleotide
gctagaggttgcttcattac

SEQ ID NO: 618 - DNA, 20 bp, Artificial Sequence
  Description of Artificial Sequence: Synthetic oligonucleotide
atattgctagaggttgcttc

SEQ ID NO: 619 - DNA, 25 bp, Artificial Sequence
  Description of Artificial Sequence: Synthetic oligonucleotide
ctttaacagaaaagcatacacatta

SEQ ID NO: 620 - DNA, 25 bp, Artificial Sequence
  Description of Artificial Sequence: Synthetic oligonucleotide
tcctctttaacagaaaagcatacac

SEQ ID NO: 621 - DNA, 25 bp, Artificial Sequence
  Description of Artificial Sequence: Synthetic oligonucleotide
ttcctctttaacagaaaagcataca

SEQ ID NO: 622 - DNA, 25 bp, Artificial Sequence
  Description of Artificial Sequence: Synthetic oligonucleotide
taacttcctctttaacagaaaagca

SEQ ID NO: 623 - DNA, 25 bp, Artificial Sequence
  Description of Artificial Sequence: Synthetic oligonucleotide
ctaacttcctctttaacagaaaagc

SEQ ID NO: 624 - DNA, 25 bp, Artificial Sequence
  Description of Artificial Sequence: Synthetic oligonucleotide
tcttctaacttcctctttaacagaa

SEQ ID NO: 625 - DNA, 25 bp, Artificial Sequence
  Description of Artificial Sequence: Synthetic oligonucleotide
atcttctaacttcctctttaacaga

SEQ ID NO: 626 - DNA, 25 bp, Artificial Sequence
  Description of Artificial Sequence: Synthetic oligonucleotide
tcagatcttctaacttcctctttaa

SEQ ID NO: 627 - DNA, 25 bp, Artificial Sequence
  Description of Artificial Sequence: Synthetic oligonucleotide
ctcagatcttctaacttcctcttta

SEQ ID NO: 628 - DNA, 25 bp, Artificial Sequence
  Description of Artificial Sequence: Synthetic oligonucleotide
agagctcagatcttctaacttcctc

SEQ ID NO: 629 - DNA, 25 bp, Artificial Sequence
  Description of Artificial Sequence: Synthetic oligonucleotide
cagagctcagatcttctaacttcct

SEQ ID NO: 630 - DNA, 24 bp, Artificial Sequence
  Description of Artificial Sequence: Synthetic oligonucleotide
cactcagagctcagatcttctact

SEQ ID NO: 631 - DNA, 25 bp, Artificial Sequence
  Description of Artificial Sequence: Synthetic oligonucleotide
ccttccactcagagctcagatcttc

SEQ ID NO: 632 - DNA, 25 bp, Artificial Sequence
  Description of Artificial Sequence: Synthetic oligonucleotide
gtaaacggtttaccgccttccactc

SEQ ID NO: 633 - DNA, 25 bp, Artificial Sequence
  Description of Artificial Sequence: Synthetic oligonucleotide
ctttgccctcagctcttgaagtaaa

SEQ ID NO: 634 - DNA, 25 bp, Artificial Sequence
  Description of Artificial Sequence: Synthetic oligonucleotide
ccctcagctcttgaagtaaacggtt

SEQ ID NO: 635 - DNA, 25 bp, Artificial Sequence
  Description of Artificial Sequence: Synthetic oligonucleotide
ccaggagctaggtcaggctgctttg

SEQ ID NO: 636 - DNA, 25 bp, Artificial Sequence
  Description of Artificial Sequence: Synthetic oligonucleotide
ggtcaggctgctttgccctcagctc

SEQ ID NO: 637 - DNA, 25 bp, Artificial Sequence
  Description of Artificial Sequence: Synthetic oligonucleotide
aggctccaatagtggtcagtccagg

SEQ ID NO: 638 - DNA, 25 bp, Artificial Sequence
  Description of Artificial Sequence: Synthetic oligonucleotide
tcagtccaggagctaggtcaggctg

SEQ ID NO: 639 - DNA, 25 bp, Artificial Sequence
  Description of Artificial Sequence: Synthetic oligonucleotide
cttacaggctccaatagtggtcagt

SEQ ID NO: 640 - DNA, 25 bp, Artificial Sequence
  Description of Artificial Sequence: Synthetic oligonucleotide
gtatacttacaggctccaatagtgg

SEQ ID NO: 641 - DNA, 25 bp, Artificial Sequence
  Description of Artificial Sequence: Synthetic oligonucleotide
atccagtatacttacaggctccaat

SEQ ID NO: 642 - DNA, 25 bp, Artificial Sequence
  Description of Artificial Sequence: Synthetic oligonucleotide
atgggatccagtatacttacaggct

SEQ ID NO: 643 - DNA, 25 bp, Artificial Sequence
  Description of Artificial Sequence: Synthetic oligonucleotide
agagaatgggatccagtatacttac

SEQ ID NO: 644 - DNA, 20 bp, Artificial Sequence
  Description of Artificial Sequence: Synthetic oligonucleotide
acagaaaagcatacacatta

SEQ ID NO: 645 - DNA, 20 bp, Artificial Sequence
  Description of Artificial Sequence: Synthetic oligonucleotide
tttaacagaaaagcatacac

SEQ ID NO: 646 - DNA, 20 bp, Artificial Sequence
  Description of Artificial Sequence: Synthetic oligonucleotide
tcctctttaacagaaaagca

SEQ ID NO: 647 - DNA, 20 bp, Artificial Sequence
  Description of Artificial Sequence: Synthetic oligonucleotide
taacttcctctttaacagaa

SEQ ID NO: 648 - DNA, 20 bp, Artificial Sequence
  Description of Artificial Sequence: Synthetic oligonucleotide
tcttctaacttcctctttaa

SEQ ID NO: 649 - DNA, 20 bp, Artificial Sequence
  Description of Artificial Sequence: Synthetic oligonucleotide
tcagatcttctaacttcctc

SEQ ID NO: 650 - DNA, 20 bp, Artificial Sequence
  Description of Artificial Sequence: Synthetic oligonucleotide
ccttccactcagagctcaga

SEQ ID NO: 651 - DNA, 20 bp, Artificial Sequence
  Description of Artificial Sequence: Synthetic oligonucleotide
gtaaacggtttaccgccttc

SEQ ID NO: 652 - DNA, 20 bp, Artificial Sequence
  Description of Artificial Sequence: Synthetic oligonucleotide
ccctcagctcttgaagtaaa

SEQ ID NO: 653 - DNA, 20 bp, Artificial Sequence
  Description of Artificial Sequence: Synthetic oligonucleotide
ggtcaggctgctttgccctc

SEQ ID NO: 654 - DNA, 20 bp, Artificial Sequence
  Description of Artificial Sequence: Synthetic oligonucleotide
tcagtccaggagctaggtca

SEQ ID NO: 655 - DNA, 20 bp, Artificial Sequence
  Description of Artificial Sequence: Synthetic oligonucleotide
aggctccaatagtggtcagt

SEQ ID NO: 656 - DNA, 20 bp, Artificial Sequence
  Description of Artificial Sequence: Synthetic oligonucleotide
cttacaggctccaatagtgg

SEQ ID NO: 657 - DNA, 20 bp, Artificial Sequence
  Description of Artificial Sequence: Synthetic oligonucleotide
gtatacttacaggctccaat

SEQ ID NO: 658 - DNA, 20 bp, Artificial Sequence
  Description of Artificial Sequence: Synthetic oligonucleotide
atccagtatacttacaggct

SEQ ID NO: 659 - DNA, 20 bp, Artificial Sequence
  Description of Artificial Sequence: Synthetic oligonucleotide
atgggatccagtatacttac

SEQ ID NO: 660 - DNA, 20 bp, Artificial Sequence
  Description of Artificial Sequence: Synthetic oligonucleotide
agagaatgggatccagtata

SEQ ID NO: 661 - DNA, 26 bp, Artificial Sequence
  Description of Artificial Sequence: Synthetic oligonucleotide
ctaaaatattttgggtttttgcaaaa

SEQ ID NO: 662 - DNA, 26 bp, Artificial Sequence
  Description of Artificial Sequence: Synthetic oligonucleotide
gctaaaatattttgggtttttgcaaa

SEQ ID NO: 663 - DNA, 26 bp, Artificial Sequence
  Description of Artificial Sequence: Synthetic oligonucleotide
taggagctaaaatattttgggttttt

SEQ ID NO: 664 - DNA, 25 bp, Artificial Sequence
  Description of Artificial Sequence: Synthetic oligonucleotide
agtaggagctaaaatattttgggtt

SEQ ID NO: 665 - DNA, 25 bp, Artificial Sequence
  Description of Artificial Sequence: Synthetic oligonucleotide
tgagtaggagctaaaatattttggg

SEQ ID NO: 666 - DNA, 26 bp, Artificial Sequence
  Description of Artificial Sequence: Synthetic oligonucleotide
ctgagtaggagctaaaatattttggg

SEQ ID NO: 667 - DNA, 25 bp, Artificial Sequence
  Description of Artificial Sequence: Synthetic oligonucleotide
cagtctgagtaggagctaaaatatt

SEQ ID NO: 668 - DNA, 26 bp, Artificial Sequence
  Description of Artificial Sequence: Synthetic oligonucleotide
acagtctgagtaggagctaaaatatt

SEQ ID NO: 669 - DNA, 26 bp, Artificial Sequence
  Description of Artificial Sequence: Synthetic oligonucleotide
gagtaacagtctgagtaggagctaaa

SEQ ID NO: 670 - DNA, 25 bp, Artificial Sequence
  Description of Artificial Sequence: Synthetic oligonucleotide
cagagtaacagtctgagtaggagct

SEQ ID NO: 671 - DNA, 26 bp, Artificial Sequence
  Description of Artificial Sequence: Synthetic oligonucleotide
caccagagtaacagtctgagtaggag

SEQ ID NO: 672 - DNA, 25 bp, Artificial Sequence
  Description of Artificial Sequence: Synthetic oligonucleotide
gtcaccagagtaacagtctgagtag

SEQ ID NO: 673 - DNA, 26 bp, Artificial Sequence
  Description of Artificial Sequence: Synthetic oligonucleotide
aaccacaggttgtgtcaccagagtaa

SEQ ID NO: 674 - DNA, 25 bp, Artificial Sequence
  Description of Artificial Sequence: Synthetic oligonucleotide
gttgtgtcaccagagtaacagtctg

SEQ ID NO: 675 - DNA, 26 bp, Artificial Sequence
  Description of Artificial Sequence: Synthetic oligonucleotide
tggcagtttccttagtaaccacaggt

SEQ ID NO: 676 - DNA, 26 bp, Artificial Sequence
  Description of Artificial Sequence: Synthetic oligonucleotide
atttctagtttggagatggcagtttc

SEQ ID NO: 677 - DNA, 25 bp, Artificial Sequence
  Description of Artificial Sequence: Synthetic oligonucleotide
ggaagatggcatttctagtttggag

SEQ ID NO: 678 - DNA, 26 bp, Artificial Sequence
  Description of Artificial Sequence: Synthetic oligonucleotide
catcaaggaagatggcatttctagtt

SEQ ID NO: 679 - DNA, 26 bp, Artificial Sequence
  Description of Artificial Sequence: Synthetic oligonucleotide
gagcaggtacctccaacatcaaggaa

SEQ ID NO: 680 - DNA, 25 bp, Artificial Sequence
  Description of Artificial Sequence: Synthetic oligonucleotide
atctgccagagcaggtacctccaac

SEQ ID NO: 681 - DNA, 26 bp, Artificial Sequence
  Description of Artificial Sequence: Synthetic oligonucleotide
aagttctgtccaagcccggttgaaat

SEQ ID NO: 682 - DNA, 26 bp, Artificial Sequence
  Description of Artificial Sequence: Synthetic oligonucleotide
cggttgaaatctgccagagcaggtac

SEQ ID NO: 683 - DNA, 26 bp, Artificial Sequence
  Description of Artificial Sequence: Synthetic oligonucleotide
gagaaagccagtcggtaagttctgtc

SEQ ID NO: 684 - DNA, 25 bp, Artificial Sequence
  Description of Artificial Sequence: Synthetic oligonucleotide
gtcggtaagttctgtccaagcccgg

SEQ ID NO: 685 - DNA, 26 bp, Artificial Sequence
  Description of Artificial Sequence: Synthetic oligonucleotide
ataacttgatcaagcagagaaagcca

SEQ ID NO: 686 - DNA, 25 bp, Artificial Sequence
  Description of Artificial Sequence: Synthetic oligonucleotide
aagcagagaaagccagtcggtaagt

SEQ ID NO: 687 - DNA, 26 bp, Artificial Sequence
  Description of Artificial Sequence: Synthetic oligonucleotide
caccctctgtgattttataacttgat

SEQ ID NO: 688 - DNA, 26 bp, Artificial Sequence
  Description of Artificial Sequence: Synthetic oligonucleotide
caaggtcacccaccatcaccctctgt

SEQ ID NO: 689 - DNA, 25 bp, Artificial Sequence
  Description of Artificial Sequence: Synthetic oligonucleotide
catcaccctctgtgattttataact

SEQ ID NO: 690 - DNA, 26 bp, Artificial Sequence
  Description of Artificial Sequence: Synthetic oligonucleotide
cttctgcttgatgatcatctcgttga

SEQ ID NO: 691 - DNA, 26 bp, Artificial Sequence
  Description of Artificial Sequence: Synthetic oligonucleotide
ccttctgcttgatgatcatctcgttg

SEQ ID NO: 692 - DNA, 26 bp, Artificial Sequence
  Description of Artificial Sequence: Synthetic oligonucleotide
atctcgttgatatcctcaaggtcacc

SEQ ID NO: 693 - DNA, 26 bp, Artificial Sequence
  Description of Artificial Sequence: Synthetic oligonucleotide
tcataccttctgcttgatgatcatct

SEQ ID NO: 694 - DNA, 26 bp, Artificial Sequence
  Description of Artificial Sequence: Synthetic oligonucleotide
tcattttttctcataccttctgcttg

SEQ ID NO: 695 - DNA, 26 bp, Artificial Sequence
  Description of Artificial Sequence: Synthetic oligonucleotide
ttttctcataccttctgcttgatgat

SEQ ID NO: 696 - DNA, 26 bp, Artificial Sequence
  Description of Artificial Sequence: Synthetic oligonucleotide
ttttatcattttttctcataccttct

SEQ ID NO: 697 - DNA, 26 bp, Artificial Sequence
  Description of Artificial Sequence: Synthetic oligonucleotide
ccaacttttatcattttttctcatac

SEQ ID NO: 698 - DNA, 20 bp, Artificial Sequence
  Description of Artificial Sequence: Synthetic oligonucleotide
atattttgggtttttgcaaa

SEQ ID NO: 699 - DNA, 20 bp, Artificial Sequence
  Description of Artificial Sequence: Synthetic oligonucleotide
aaaatattttgggtttttgc

SEQ ID NO: 700 - DNA, 20 bp, Artificial Sequence
  Description of Artificial Sequence: Synthetic oligonucleotide
gagctaaaatattttgggtt

SEQ ID NO: 701 - DNA, 20 bp, Artificial Sequence
  Description of Artificial Sequence: Synthetic oligonucleotide
agtaggagctaaaatatttt

SEQ ID NO: 702 - DNA, 20 bp, Artificial Sequence
  Description of Artificial Sequence: Synthetic oligonucleotide
gtctgagtaggagctaaaat

SEQ ID NO: 703 - DNA, 20 bp, Artificial Sequence
  Description of Artificial Sequence: Synthetic oligonucleotide
taacagtctgagtaggagct

SEQ ID NO: 704 - DNA, 20 bp, Artificial Sequence
  Description of Artificial Sequence: Synthetic oligonucleotide
cagagtaacagtctgagtag

SEQ ID NO: 705 - DNA, 20 bp, Artificial Sequence
  Description of Artificial Sequence: Synthetic oligonucleotide
cacaggttgtgtcaccagag

SEQ ID NO: 706 - DNA, 20 bp, Artificial Sequence
  Description of Artificial Sequence: Synthetic oligonucleotide
agtttccttagtaaccacag

SEQ ID NO: 707 - DNA, 20 bp, Artificial Sequence
  Description of Artificial Sequence: Synthetic oligonucleotide
tagtttggagatggcagttt

SEQ ID NO: 708 - DNA, 20 bp, Artificial Sequence
  Description of Artificial Sequence: Synthetic oligonucleotide
ggaagatggcatttctagtt

SEQ ID NO: 709 - DNA, 20 bp, Artificial Sequence
  Description of Artificial Sequence: Synthetic oligonucleotide
tacctccaacatcaaggaag

SEQ ID NO: 710 - DNA, 20 bp, Artificial Sequence
  Description of Artificial Sequence: Synthetic oligonucleotide
atctgccagagcaggtacct

SEQ ID NO: 711 - DNA, 20 bp, Artificial Sequence
  Description of Artificial Sequence: Synthetic oligonucleotide
ccaagcccggttgaaatctg

SEQ ID NO: 712 - DNA, 20 bp, Artificial Sequence
  Description of Artificial Sequence: Synthetic oligonucleotide
gtcggtaagttctgtccaag

SEQ ID NO: 713 - DNA, 20 bp, Artificial Sequence
  Description of Artificial Sequence: Synthetic oligonucleotide
aagcagagaaagccagtcgg

SEQ ID NO: 714 - DNA, 20 bp, Artificial Sequence
  Description of Artificial Sequence: Synthetic oligonucleotide
ttttataacttgatcaagca

SEQ ID NO: 715 - DNA, 20 bp, Artificial Sequence
  Description of Artificial Sequence: Synthetic oligonucleotide
catcaccctctgtgatttta

SEQ ID NO: 716 - DNA, 20 bp, Artificial Sequence
  Description of Artificial Sequence: Synthetic oligonucleotide
ctcaaggtcacccaccatca

SEQ ID NO: 717 - DNA, 20 bp, Artificial Sequence
  Description of Artificial Sequence: Synthetic oligonucleotide
catctcgttgatatcctcaa

SEQ ID NO: 718 - DNA, 20 bp, Artificial Sequence
  Description of Artificial Sequence: Synthetic oligonucleotide
cttctgcttgatgatcatct

SEQ ID NO: 719 - DNA, 20 bp, Artificial Sequence
  Description of Artificial Sequence: Synthetic oligonucleotide
cataccttctgcttgatgat

SEQ ID NO: 720 - DNA, 20 bp, Artificial Sequence
  Description of Artificial Sequence: Synthetic oligonucleotide
tttctcataccttctgcttg

SEQ ID NO: 721 - DNA, 20 bp, Artificial Sequence
  Description of Artificial Sequence: Synthetic oligonucleotide
cattttttctcataccttct

SEQ ID NO: 722 - DNA, 20 bp, Artificial Sequence
  Description of Artificial Sequence: Synthetic oligonucleotide
tttatcattttttctcatac

SEQ ID NO: 723 - DNA, 20 bp, Artificial Sequence
  Description of Artificial Sequence: Synthetic oligonucleotide
caacttttatcattttttct

SEQ ID NO: 724 - DNA, 25 bp, Artificial Sequence
  Description of Artificial Sequence: Synthetic oligonucleotide
ctgtaagaacaaatatcccttagta

SEQ ID NO: 725 - DNA, 25 bp, Artificial Sequence
  Description of Artificial Sequence: Synthetic oligonucleotide
tgcctgtaagaacaaatatccctta

SEQ ID NO: 726 - DNA, 25 bp, Artificial Sequence
  Description of Artificial Sequence: Synthetic oligonucleotide
gttgcctgtaagaacaaatatccct

SEQ ID NO: 727 - DNA, 25 bp, Artificial Sequence
  Description of Artificial Sequence: Synthetic oligonucleotide
attgttgcctgtaagaacaaatatc

SEQ ID NO: 728 - DNA, 25 bp, Artificial Sequence
  Description of Artificial Sequence: Synthetic oligonucleotide
gcattgttgcctgtaagaacaaata

SEQ ID NO: 729 - DNA, 25 bp, Artificial Sequence
  Description of Artificial Sequence: Synthetic oligonucleotide
cctgcattgttgcctgtaagaacaa

SEQ ID NO: 730 - DNA, 25 bp, Artificial Sequence
  Description of Artificial Sequence: Synthetic oligonucleotide
atcctgcattgttgcctgtaagaac

SEQ ID NO: 731 - DNA, 25 bp, Artificial Sequence
  Description of Artificial Sequence: Synthetic oligonucleotide
caaatcctgcattgttgcctgtaag

SEQ ID NO: 732 - DNA, 25 bp, Artificial Sequence
  Description of Artificial Sequence: Synthetic oligonucleotide
tccaaatcctgcattgttgcctgta

SEQ ID NO: 733 - DNA, 25 bp, Artificial Sequence
  Description of Artificial Sequence: Synthetic oligonucleotide
tgttccaaatcctgcattgttgcct

SEQ ID NO: 734 - DNA, 25 bp, Artificial Sequence
  Description of Artificial Sequence: Synthetic oligonucleotide
tctgttccaaatcctgcattgttgc

SEQ ID NO: 735 - DNA, 25 bp, Artificial Sequence
  Description of Artificial Sequence: Synthetic oligonucleotide
aactggggacgcctctgttccaaat

SEQ ID NO: 736 - DNA, 25 bp, Artificial Sequence
  Description of Artificial Sequence: Synthetic oligonucleotide
gcctctgttccaaatcctgcattgt

SEQ ID NO: 737 - DNA, 25 bp, Artificial Sequence
  Description of Artificial Sequence: Synthetic oligonucleotide
cagcggtaatgagttcttccaactg

SEQ ID NO: 738 - DNA, 25 bp, Artificial Sequence
  Description of Artificial Sequence: Synthetic oligonucleotide
cttccaactggggacgcctctgttc

SEQ ID NO: 739 - DNA, 25 bp, Artificial Sequence
  Description of Artificial Sequence: Synthetic oligonucleotide
cttgtttttcaaattttgggcagcg

SEQ ID NO: 740 - DNA, 25 bp, Artificial Sequence
  Description of Artificial Sequence: Synthetic oligonucleotide
ctagcctcttgattgctggtcttgt

SEQ ID NO: 741 - DNA, 25 bp, Artificial Sequence
  Description of Artificial Sequence: Synthetic oligonucleotide
ttttcaaattttgggcagcggtaat

SEQ ID NO: 742 - DNA, 25 bp, Artificial Sequence
  Description of Artificial Sequence: Synthetic oligonucleotide
ttcgatccgtaatgattgttctagc

SEQ ID NO: 743 - DNA, 25 bp, Artificial Sequence
  Description of Artificial Sequence: Synthetic oligonucleotide
gattgctggtcttgtttttcaaatt

SEQ ID NO: 744 - DNA, 25 bp, Artificial Sequence
  Description of Artificial Sequence: Synthetic oligonucleotide
cttacttcgatccgtaatgattgtt

SEQ ID NO: 745 - DNA, 25 bp, Artificial Sequence
  Description of Artificial Sequence: Synthetic oligonucleotide
ttgttctagcctcttgattgctggt

SEQ ID NO: 746 - DNA, 25 bp, Artificial Sequence
  Description of Artificial Sequence: Synthetic oligonucleotide
aaaaacttacttcgatccgtaatga

SEQ ID NO: 747 - DNA, 25 bp, Artificial Sequence
  Description of Artificial Sequence: Synthetic oligonucleotide
tgttaaaaaacttacttcgatccgt

SEQ ID NO: 748 - DNA, 25 bp, Artificial Sequence
  Description of Artificial Sequence: Synthetic oligonucleotide
atgcttgttaaaaaacttacttcga

SEQ ID NO: 749 - DNA, 25 bp, Artificial Sequence
  Description of Artificial Sequence: Synthetic oligonucleotide
gtcccatgcttgttaaaaaacttac

SEQ ID NO: 750 - DNA, 20 bp, Artificial Sequence
  Description of Artificial Sequence: Synthetic oligonucleotide
agaacaaatatcccttagta

SEQ ID NO: 751 - DNA, 20 bp, Artificial Sequence
  Description of Artificial Sequence: Synthetic oligonucleotide
gtaagaacaaatatccctta

SEQ ID NO: 752 - DNA, 20 bp, Artificial Sequence
  Description of Artificial Sequence: Synthetic oligonucleotide
tgcctgtaagaacaaatatc

SEQ ID NO: 753 - DNA, 20 bp, Artificial Sequence
  Description of Artificial Sequence: Synthetic oligonucleotide
attgttgcctgtaagaacaa

SEQ ID NO: 754 - DNA, 20 bp, Artificial Sequence
  Description of Artificial Sequence: Synthetic oligonucleotide
cctgcattgttgcctgtaag

SEQ ID NO: 755 - DNA, 20 bp, Artificial Sequence
  Description of Artificial Sequence: Synthetic oligonucleotide
caaatcctgcattgttgcct

SEQ ID NO: 756 - DNA, 20 bp, Artificial Sequence
  Description of Artificial Sequence: Synthetic oligonucleotide
gcctctgttccaaatcctgc

SEQ ID NO: 757 - DNA, 20 bp, Artificial Sequence
  Description of Artificial Sequence: Synthetic oligonucleotide
cttccaactggggacgcctc

SEQ ID NO: 758 - DNA, 20 bp, Artificial Sequence
  Description of Artificial Sequence: Synthetic oligonucleotide
cagcggtaatgagttcttcc

SEQ ID NO: 759 - DNA, 20 bp, Artificial Sequence
  Description of Artificial Sequence: Synthetic oligonucleotide
ttttcaaattttgggcagcg

SEQ ID NO: 760 - DNA, 20 bp, Artificial Sequence
  Description of Artificial Sequence: Synthetic oligonucleotide
gattgctggtcttgtttttc

SEQ ID NO: 761 - DNA, 20 bp, Artificial Sequence
  Description of Artificial Sequence: Synthetic oligonucleotide
ttgttctagcctcttgattg

SEQ ID NO: 762 - DNA, 20 bp, Artificial Sequence
  Description of Artificial Sequence: Synthetic oligonucleotide
ttcgatccgtaatgattgtt

SEQ ID NO: 763 - DNA, 20 bp, Artificial Sequence
  Description of Artificial Sequence: Synthetic oligonucleotide
cttacttcgatccgtaatga

SEQ ID NO: 764 - DNA, 20 bp, Artificial Sequence
  Description of Artificial Sequence: Synthetic oligonucleotide
aaaaacttacttcgatccgt

SEQ ID NO: 765 - DNA, 20 bp, Artificial Sequence
  Description of Artificial Sequence: Synthetic oligonucleotide
tgttaaaaaacttacttcga

SEQ ID NO: 766 - DNA, 20 bp, Artificial Sequence
  Description of Artificial Sequence: Synthetic oligonucleotide
atgcttgttaaaaaacttac

SEQ ID NO: 767 - DNA, 20 bp, Artificial Sequence
  Description of Artificial Sequence: Synthetic oligonucleotide
gtcccatgcttgttaaaaaa

SEQ ID NO: 768 - DNA, 25 bp, Artificial Sequence
  Description of Artificial Sequence: Synthetic oligonucleotide
ctagaataaaaggaaaaataaatat

SEQ ID NO: 769 - DNA, 25 bp, Artificial Sequence
  Description of Artificial Sequence: Synthetic oligonucleotide
aactagaataaaaggaaaaataaat

SEQ ID NO: 770 - DNA, 25 bp, Artificial Sequence
  Description of Artificial Sequence: Synthetic oligonucleotide
ttcaactagaataaaaggaaaaata

SEQ ID NO: 771 - DNA, 25 bp, Artificial Sequence
  Description of Artificial Sequence: Synthetic oligonucleotide
ctttcaactagaataaaaggaaaaa

SEQ ID NO: 772 - DNA, 25 bp, Artificial Sequence
  Description of Artificial Sequence: Synthetic oligonucleotide
attctttcaactagaataaaaggaa

SEQ ID NO: 773 - DNA, 25 bp, Artificial Sequence
  Description of Artificial Sequence: Synthetic oligonucleotide
gaattctttcaactagaataaaagg

SEQ ID NO: 774 - DNA, 25 bp, Artificial Sequence
  Description of Artificial Sequence: Synthetic oligonucleotide
tctgaattctttcaactagaataaa

SEQ ID NO: 775 - DNA, 25 bp, Artificial Sequence
  Description of Artificial Sequence: Synthetic oligonucleotide
attctgaattctttcaactagaata

SEQ ID NO: 776 - DNA, 25 bp, Artificial Sequence
  Description of Artificial Sequence: Synthetic oligonucleotide
ctgattctgaattctttcaactaga

SEQ ID NO: 777 - DNA, 25 bp, Artificial Sequence
  Description of Artificial Sequence: Synthetic oligonucleotide
cactgattctgaattctttcaacta

SEQ ID NO: 778 - DNA, 25 bp, Artificial Sequence
  Description of Artificial Sequence: Synthetic oligonucleotide
tcccactgattctgaattctttcaa

SEQ ID NO: 779 - DNA, 25 bp, Artificial Sequence
  Description of Artificial Sequence: Synthetic oligonucleotide
catcccactgattctgaattctttc

SEQ ID NO: 780 - DNA, 25 bp, Artificial Sequence
  Description of Artificial Sequence: Synthetic oligonucleotide
tacttcatcccactgattctgaatt

SEQ ID NO: 781 - DNA, 25 bp, Artificial Sequence
  Description of Artificial Sequence: Synthetic oligonucleotide
ctgaaggtgttcttgtacttcatcc

SEQ ID NO: 782 - DNA, 24 bp, Artificial Sequence
  Description of Artificial Sequence: Synthetic oligonucleotide
cggttctgaaggtgttcttgtact

SEQ ID NO: 783 - DNA, 25 bp, Artificial Sequence
  Description of Artificial Sequence: Synthetic oligonucleotide
ctgttgcctccggttctgaaggtgt

SEQ ID NO: 784 - DNA, 25 bp, Artificial Sequence
  Description of Artificial Sequence: Synthetic oligonucleotide
tttcattcaactgttgcctccggtt

SEQ ID NO: 785 - DNA, 25 bp, Artificial Sequence
  Description of Artificial Sequence: Synthetic oligonucleotide
taacatttcattcaactgttgcctc

SEQ ID NO: 786 - DNA, 25 bp, Artificial Sequence
  Description of Artificial Sequence: Synthetic oligonucleotide
ttgtgttgaatcctttaacatttca

SEQ ID NO: 787 - DNA, 25 bp, Artificial Sequence
  Description of Artificial Sequence: Synthetic oligonucleotide
tcttccttagcttccagccattgtg

SEQ ID NO: 788 - DNA, 25 bp, Artificial Sequence
  Description of Artificial Sequence: Synthetic oligonucleotide
cttagcttccagccattgtgttgaa

SEQ ID NO: 789 - DNA, 25 bp, Artificial Sequence
  Description of Artificial Sequence: Synthetic oligonucleotide
gtcctaagacctgctcagcttcttc

SEQ ID NO: 790 - DNA, 25 bp, Artificial Sequence
  Description of Artificial Sequence: Synthetic oligonucleotide
ctgctcagcttcttccttagcttcc

SEQ ID NO: 791 - DNA, 25 bp, Artificial Sequence
  Description of Artificial Sequence: Synthetic oligonucleotide
ctcaagcttggctctggcctgtcct

SEQ ID NO: 792 - DNA, 25 bp, Artificial Sequence
  Description of Artificial Sequence: Synthetic oligonucleotide
ggcctgtcctaagacctgctcagct

SEQ ID NO: 793 - DNA, 25 bp, Artificial Sequence
  Description of Artificial Sequence: Synthetic oligonucleotide
tagggaccctccttccatgactcaa

SEQ ID NO: 794 - DNA, 25 bp, Artificial Sequence
  Description of Artificial Sequence: Synthetic oligonucleotide
tttggattgcatctactgtataggg

SEQ ID NO: 795 - DNA, 25 bp, Artificial Sequence
  Description of Artificial Sequence: Synthetic oligonucleotide
accctccttccatgactcaagcttg

SEQ ID NO: 796 - DNA, 25 bp, Artificial Sequence
  Description of Artificial Sequence: Synthetic oligonucleotide
cttggtttctgtgattttcttttgg

SEQ ID NO: 797 - DNA, 25 bp, Artificial Sequence
  Description of Artificial Sequence: Synthetic oligonucleotide
atctactgtatagggaccctccttc

SEQ ID NO: 798 - DNA, 25 bp, Artificial Sequence
  Description of Artificial Sequence: Synthetic oligonucleotide
ctaaccttggtttctgtgattttct

SEQ ID NO: 799 - DNA, 25 bp, Artificial Sequence
  Description of Artificial Sequence: Synthetic oligonucleotide
tttcttttggattgcatctactgta

SEQ ID NO: 800 - DNA, 25 bp, Artificial Sequence
  Description of Artificial Sequence: Synthetic oligonucleotide
tgatactaaccttggtttctgtgat

SEQ ID NO: 801 - DNA, 25 bp, Artificial Sequence
  Description of Artificial Sequence: Synthetic oligonucleotide
atctttgatactaaccttggtttct

SEQ ID NO: 802 - DNA, 25 bp, Artificial Sequence
  Description of Artificial Sequence: Synthetic oligonucleotide
aaggtatctttgatactaaccttgg

SEQ ID NO: 803 - DNA, 25 bp, Artificial Sequence
  Description of Artificial Sequence: Synthetic oligonucleotide
ttaaaaaggtatctttgatactaac

SEQ ID NO: 804 - DNA, 20 bp, Artificial Sequence
  Description of Artificial Sequence: Synthetic oligonucleotide
ataaaaggaaaaataaatat

SEQ ID NO: 805 - DNA, 20 bp, Artificial Sequence
  Description of Artificial Sequence: Synthetic oligonucleotide
gaataaaaggaaaaataaat

SEQ ID NO: 806 - DNA, 20 bp, Artificial Sequence
  Description of Artificial Sequence: Synthetic oligonucleotide
aactagaataaaaggaaaaa

SEQ ID NO: 807 - DNA, 20 bp, Artificial Sequence
  Description of Artificial Sequence: Synthetic oligonucleotide
ctttcaactagaataaaagg

SEQ ID NO: 808 - DNA, 20 bp, Artificial Sequence
  Description of Artificial Sequence: Synthetic oligonucleotide
gaattctttcaactagaata

SEQ ID NO: 809 - DNA, 20 bp, Artificial Sequence
  Description of Artificial Sequence: Synthetic oligonucleotide
attctgaattctttcaacta

SEQ ID NO: 810 - DNA, 20 bp, Artificial Sequence
  Description of Artificial Sequence: Synthetic oligonucleotide
tacttcatcccactgattct

SEQ ID NO: 811 - DNA, 19 bp, Artificial Sequence
  Description of Artificial Sequence: Synthetic oligonucleotide
ctgaaggtgttcttgtact

SEQ ID NO: 812 - DNA, 20 bp, Artificial Sequence
  Description of Artificial Sequence: Synthetic oligonucleotide
ctgttgcctccggttctgaa

SEQ ID NO: 813 - DNA, 20 bp, Artificial Sequence
  Description of Artificial Sequence: Synthetic oligonucleotide
taacatttcattcaactgtt

SEQ ID NO: 814 - DNA, 20 bp, Artificial Sequence
  Description of Artificial Sequence: Synthetic oligonucleotide
ttgtgttgaatcctttaaca

SEQ ID NO: 815 - DNA, 20 bp, Artificial Sequence
  Description of Artificial Sequence: Synthetic oligonucleotide
cttagcttccagccattgtg

SEQ ID NO: 816 - DNA, 20 bp, Artificial Sequence
  Description of Artificial Sequence: Synthetic oligonucleotide
ctgctcagcttcttccttag

SEQ ID NO: 817 - DNA, 20 bp, Artificial Sequence
  Description of Artificial Sequence: Synthetic oligonucleotide
ggcctgtcctaagacctgct

SEQ ID NO: 818 - DNA, 20 bp, Artificial Sequence
  Description of Artificial Sequence: Synthetic oligonucleotide
ctcaagcttggctctggcct

SEQ ID NO: 819 - DNA, 20 bp, Artificial Sequence
  Description of Artificial Sequence: Synthetic oligonucleotide
accctccttccatgactcaa

SEQ ID NO: 820 - DNA, 20 bp, Artificial Sequence
  Description of Artificial Sequence: Synthetic oligonucleotide
atctactgtatagggaccct

SEQ ID NO: 821 - DNA, 20 bp, Artificial Sequence
  Description of Artificial Sequence: Synthetic oligonucleotide
tttcttttggattgcatcta

SEQ ID NO: 822 - DNA, 20 bp, Artificial Sequence
  Description of Artificial Sequence: Synthetic oligonucleotide
cttggtttctgtgattttct

SEQ ID NO: 823 - DNA, 20 bp, Artificial Sequence
  Description of Artificial Sequence: Synthetic oligonucleotide
ctaaccttggtttctgtgat

SEQ ID NO: 824 - DNA, 20 bp, Artificial Sequence
  Description of Artificial Sequence: Synthetic oligonucleotide
tgatactaaccttggtttct

SEQ ID NO: 825 - DNA, 20 bp, Artificial Sequence
  Description of Artificial Sequence: Synthetic oligonucleotide
atctttgatactaaccttgg

SEQ ID NO: 826 - DNA, 20 bp, Artificial Sequence
  Description of Artificial Sequence: Synthetic oligonucleotide
aaggtatctttgatactaac

SEQ ID NO: 827 - DNA, 20 bp, Artificial Sequence
  Description of Artificial Sequence: Synthetic oligonucleotide
ttaaaaaggtatctttgata

SEQ ID NO: 828 - DNA, 24 bp, Artificial Sequence
  Description of Artificial Sequence: Synthetic oligonucleotide
ctatagatttttatgagaaagaga

SEQ ID NO: 829 - DNA, 25 bp, Artificial Sequence
  Description of Artificial Sequence: Synthetic oligonucleotide
aactgctatagatttttatgagaaa

SEQ ID NO: 830 - DNA, 25 bp, Artificial Sequence
  Description of Artificial Sequence: Synthetic oligonucleotide
tggccaactgctatagatttttatg

SEQ ID NO: 831 - DNA, 25 bp, Artificial Sequence
  Description of Artificial Sequence: Synthetic oligonucleotide
gtctttggccaactgctatagattt

SEQ ID NO: 832 - DNA, 25 bp, Artificial Sequence
  Description of Artificial Sequence: Synthetic oligonucleotide
cggaggtctttggccaactgctata

SEQ ID NO: 833 - DNA, 25 bp, Artificial Sequence
  Description of Artificial Sequence: Synthetic oligonucleotide
actggcggaggtctttggccaactg

SEQ ID NO: 834 - DNA, 25 bp, Artificial Sequence
  Description of Artificial Sequence: Synthetic oligonucleotide
tttgtctgccactggcggaggtctt

SEQ ID NO: 835 - DNA, 25 bp, Artificial Sequence
  Description of Artificial Sequence: Synthetic oligonucleotide
agtcatttgccacatctacatttgt

SEQ ID NO: 836 - DNA, 25 bp, Artificial Sequence
  Description of Artificial Sequence: Synthetic oligonucleotide
tttgccacatctacatttgtctgcc

SEQ ID NO: 837 - DNA, 25 bp, Artificial Sequence
  Description of Artificial Sequence: Synthetic oligonucleotide
ccggagaagtttcagggccaagtca

SEQ ID NO: 838 - DNA, 25 bp, Artificial Sequence
  Description of Artificial Sequence: Synthetic oligonucleotide
gtatcatctgcagaataatcccgga

SEQ ID NO: 839 - DNA, 25 bp, Artificial Sequence
  Description of Artificial Sequence: Synthetic oligonucleotide
taatcccggagaagtttcagggcca

SEQ ID NO: 840 - DNA, 25 bp, Artificial Sequence
  Description of Artificial Sequence: Synthetic oligonucleotide
ttatcatgtggacttttctggtatc

SEQ ID NO: 841 - DNA, 25 bp, Artificial Sequence
  Description of Artificial Sequence: Synthetic oligonucleotide
agaggcattgatattctctgttatc

SEQ ID NO: 842 - DNA, 25 bp, Artificial Sequence
  Description of Artificial Sequence: Synthetic oligonucleotide
atgtggacttttctggtatcatctg

SEQ ID NO: 843 - DNA, 25 bp, Artificial Sequence
  Description of Artificial Sequence: Synthetic oligonucleotide
cttttatgaatgcttctccaagagg

SEQ ID NO: 844 - DNA, 25 bp, Artificial Sequence
  Description of Artificial Sequence: Synthetic oligonucleotide
atattctctgttatcatgtggactt

SEQ ID NO: 845 - DNA, 25 bp, Artificial Sequence
  Description of Artificial Sequence: Synthetic oligonucleotide
cataccttttatgaatgcttctcca

SEQ ID NO: 846 - DNA, 25 bp, Artificial Sequence
  Description of Artificial Sequence: Synthetic oligonucleotide
ctccaagaggcattgatattctctg

SEQ ID NO: 847 - DNA, 25 bp, Artificial Sequence
  Description of Artificial Sequence: Synthetic oligonucleotide
taattcataccttttatgaatgctt

SEQ ID NO: 848 - DNA, 25 bp, Artificial Sequence
  Description of Artificial Sequence: Synthetic oligonucleotide
cttttatgaatgcttctccaagagg

SEQ ID NO: 849 - DNA, 25 bp, Artificial Sequence
  Description of Artificial Sequence: Synthetic oligonucleotide
taatgtaattcataccttttatgaa

SEQ ID NO: 850 - DNA, 25 bp, Artificial Sequence
  Description of Artificial Sequence: Synthetic oligonucleotide
agaaataatgtaattcatacctttt

SEQ ID NO: 851 - DNA, 25 bp, Artificial Sequence
  Description of Artificial Sequence: Synthetic oligonucleotide
gttttagaaataatgtaattcatac

SEQ ID NO: 852 - DNA, 19 bp, Artificial Sequence
  Description of Artificial Sequence: Synthetic oligonucleotide
gatttttatgagaaagaga

SEQ ID NO: 853 - DNA, 20 bp, Artificial Sequence
  Description of Artificial Sequence: Synthetic oligonucleotide
ctatagatttttatgagaaa

SEQ ID NO: 854 - DNA, 20 bp, Artificial Sequence
  Description of Artificial Sequence: Synthetic oligonucleotide
aactgctatagatttttatg

SEQ ID NO: 855 - DNA, 20 bp, Artificial Sequence
  Description of Artificial Sequence: Synthetic oligonucleotide
tggccaactgctatagattt

SEQ ID NO: 856 - DNA, 20 bp, Artificial Sequence
  Description of Artificial Sequence: Synthetic oligonucleotide
gtctttggccaactgctata

SEQ ID NO: 857 - DNA, 20 bp, Artificial Sequence
  Description of Artificial Sequence: Synthetic oligonucleotide
cggaggtctttggccaactg

SEQ ID NO: 858 - DNA, 20 bp, Artificial Sequence
  Description of Artificial Sequence: Synthetic oligonucleotide
tttgtctgccactggcggag

SEQ ID NO: 859 - DNA, 20 bp, Artificial Sequence
  Description of Artificial Sequence: Synthetic oligonucleotide
tttgccacatctacatttgt

SEQ ID NO: 860 - DNA, 20 bp, Artificial Sequence
  Description of Artificial Sequence: Synthetic oligonucleotide
ttcagggccaagtcatttgc

SEQ ID NO: 861 - DNA, 20 bp, Artificial Sequence
  Description of Artificial Sequence: Synthetic oligonucleotide
taatcccggagaagtttcag

SEQ ID NO: 862 - DNA, 20 bp, Artificial Sequence
  Description of Artificial Sequence: Synthetic oligonucleotide
gtatcatctgcagaataatc

SEQ ID NO: 863 - DNA, 20 bp, Artificial Sequence
  Description of Artificial Sequence: Synthetic oligonucleotide
atgtggacttttctggtatc

SEQ ID NO: 864 - DNA, 20 bp, Artificial Sequence
  Description of Artificial Sequence: Synthetic oligonucleotide
atattctctgttatcatgtg

SEQ ID NO: 865 - DNA, 20 bp, Artificial Sequence
  Description of Artificial Sequence: Synthetic oligonucleotide
ctccaagaggcattgatatt

SEQ ID NO: 866 - DNA, 20 bp, Artificial Sequence
  Description of Artificial Sequence: Synthetic oligonucleotide
cttttatgaatgcttctcca

SEQ ID NO: 867 - DNA, 20 bp, Artificial Sequence
  Description of Artificial Sequence: Synthetic oligonucleotide
cataccttttatgaatgctt

SEQ ID NO: 868 - DNA, 20 bp, Artificial Sequence
  Description of Artificial Sequence: Synthetic oligonucleotide
taattcataccttttatgaa

SEQ ID NO: 869 - DNA, 20 bp, Artificial Sequence
  Description of Artificial Sequence: Synthetic oligonucleotide
taatgtaattcatacctttt

SEQ ID NO: 870 - DNA, 20 bp, Artificial Sequence
  Description of Artificial Sequence: Synthetic oligonucleotide
agaaataatgtaattcatac

SEQ ID NO: 871 - DNA, 20 bp, Artificial Sequence
  Description of Artificial Sequence: Synthetic oligonucleotide
gttttagaaataatgtaatt

SEQ ID NO: 872 - DNA, 25 bp, Artificial Sequence
  Description of Artificial Sequence: Synthetic oligonucleotide
ctgcaaaggaccaaatgttcagatg

SEQ ID NO: 873 - DNA, 25 bp, Artificial Sequence
  Description of Artificial Sequence: Synthetic oligonucleotide
tcaccctgcaaaggaccaaatgttc

SEQ ID NO: 874 - DNA, 25 bp, Artificial Sequence
  Description of Artificial Sequence: Synthetic oligonucleotide
ctcactcaccctgcaaaggaccaaa

SEQ ID NO: 875 - DNA, 25 bp, Artificial Sequence
  Description of Artificial Sequence: Synthetic oligonucleotide
tctcgctcactcaccctgcaaagga

SEQ ID NO: 876 - DNA, 25 bp, Artificial Sequence
  Description of Artificial Sequence: Synthetic oligonucleotide
cagcctctcgctcactcaccctgca

SEQ ID NO: 877 - DNA, 25 bp, Artificial Sequence
  Description of Artificial Sequence: Synthetic oligonucleotide
caaagcagcctctcgctcactcacc

SEQ ID NO: 878 - DNA, 25 bp, Artificial Sequence
  Description of Artificial Sequence: Synthetic oligonucleotide
tcttccaaagcagcctctcgctcac

SEQ ID NO: 879 - DNA, 25 bp, Artificial Sequence
  Description of Artificial Sequence: Synthetic oligonucleotide
tctatgagtttcttccaaagcagcc

SEQ ID NO: 880 - DNA, 25 bp, Artificial Sequence
  Description of Artificial Sequence: Synthetic oligonucleotide
gttgcagtaatctatgagtttcttc

SEQ ID NO: 881 - DNA, 25 bp, Artificial Sequence
  Description of Artificial Sequence: Synthetic oligonucleotide
gaactgttgcagtaatctatgagtt

SEQ ID NO: 882 - DNA, 25 bp, Artificial Sequence
  Description of Artificial Sequence: Synthetic oligonucleotide
ttccaggtccagggggaactgttgc

SEQ ID NO: 883 - DNA, 25 bp, Artificial Sequence
  Description of Artificial Sequence: Synthetic oligonucleotide
gtaagccaggcaagaaacttttcca

SEQ ID NO: 884 - DNA, 25 bp, Artificial Sequence
  Description of Artificial Sequence: Synthetic oligonucleotide
ccaggcaagaaacttttccaggtcc

SEQ ID NO: 885 - DNA, 25 bp, Artificial Sequence
  Description of Artificial Sequence: Synthetic oligonucleotide
tggcagttgtttcagcttctgtaag

SEQ ID NO: 886 - DNA, 25 bp, Artificial Sequence
  Description of Artificial Sequence: Synthetic oligonucleotide
ttcagcttctgtaagccaggcaaga

SEQ ID NO: 887 - DNA, 25 bp, Artificial Sequence
  Description of Artificial Sequence: Synthetic oligonucleotide
ggtagcatcctgtaggacattggca

SEQ ID NO: 888 - DNA, 25 bp, Artificial Sequence
  Description of Artificial Sequence: Synthetic oligonucleotide
gacattggcagttgtttcagcttct

SEQ ID NO: 889 - DNA, 25 bp, Artificial Sequence
  Description of Artificial Sequence: Synthetic oligonucleotide
tctaggagcctttccttacgggtag

SEQ ID NO: 890 - DNA, 25 bp, Artificial Sequence
  Description of Artificial Sequence: Synthetic oligonucleotide
cttttactcccttggagtcttctag

SEQ ID NO: 891 - DNA, 25 bp, Artificial Sequence
  Description of Artificial Sequence: Synthetic oligonucleotide
gagcctttccttacgggtagcatcc

SEQ ID NO: 892 - DNA, 25 bp, Artificial Sequence
  Description of Artificial Sequence: Synthetic oligonucleotide
ttgccattgtttcatcagctctttt

SEQ ID NO: 893 - DNA, 25 bp, Artificial Sequence
  Description of Artificial Sequence: Synthetic oligonucleotide
cttggagtcttctaggagcctttcc

SEQ ID NO: 894 - DNA, 25 bp, Artificial Sequence
  Description of Artificial Sequence: Synthetic oligonucleotide
cttacttgccattgtttcatcagct

SEQ ID NO: 895 - DNA, 25 bp, Artificial Sequence
  Description of Artificial Sequence: Synthetic oligonucleotide
cagctcttttactcccttggagtct

SEQ ID NO: 896 - DNA, 25 bp, Artificial Sequence
  Description of Artificial Sequence: Synthetic oligonucleotide
cctgacttacttgccattgtttcat

SEQ ID NO: 897 - DNA, 25 bp, Artificial Sequence
  Description of Artificial Sequence: Synthetic oligonucleotide
aaatgcctgacttacttgccattgt

SEQ ID NO: 898 - DNA, 25 bp, Artificial Sequence
  Description of Artificial Sequence: Synthetic oligonucleotide
agcggaaatgcctgacttacttgcc

SEQ ID NO: 899 - DNA, 25 bp, Artificial Sequence
  Description of Artificial Sequence: Synthetic oligonucleotide
gctaaagcggaaatgcctgacttac

SEQ ID NO: 900 - DNA, 20 bp, Artificial Sequence
  Description of Artificial Sequence: Synthetic oligonucleotide
aaggaccaaatgttcagatg

SEQ ID NO: 901 - DNA, 20 bp, Artificial Sequence
  Description of Artificial Sequence: Synthetic oligonucleotide
ctgcaaaggaccaaatgttc

SEQ ID NO: 902 - DNA, 20 bp, Artificial Sequence
  Description of Artificial Sequence: Synthetic oligonucleotide
tcaccctgcaaaggaccaaa

SEQ ID NO: 903 - DNA, 20 bp, Artificial Sequence
  Description of Artificial Sequence: Synthetic oligonucleotide
ctcactcaccctgcaaagga

SEQ ID NO: 904 - DNA, 20 bp, Artificial Sequence
  Description of Artificial Sequence: Synthetic oligonucleotide
tctcgctcactcaccctgca

SEQ ID NO: 905 - DNA, 20 bp, Artificial Sequence
  Description of Artificial Sequence: Synthetic oligonucleotide
cagcctctcgctcactcacc

SEQ ID NO: 906 - DNA, 20 bp, Artificial Sequence
  Description of Artificial Sequence: Synthetic oligonucleotide
caaagcagcctctcgctcac

SEQ ID NO: 907 - DNA, 20 bp, Artificial Sequence
  Description of Artificial Sequence: Synthetic oligonucleotide
tctatgagtttcttccaaag

SEQ ID NO: 908 - DNA, 20 bp, Artificial Sequence
  Description of Artificial Sequence: Synthetic oligonucleotide
gaactgttgcagtaatctat

SEQ ID NO: 909 - DNA, 20 bp, Artificial Sequence
  Description of Artificial Sequence: Synthetic oligonucleotide
ttccaggtccagggggaact

SEQ ID NO: 910 - DNA, 20 bp, Artificial Sequence
  Description of Artificial Sequence: Synthetic oligonucleotide
ccaggcaagaaacttttcca

SEQ ID NO: 911 - DNA, 20 bp, Artificial Sequence
  Description of Artificial Sequence: Synthetic oligonucleotide
ttcagcttctgtaagccagg

SEQ ID NO: 912 - DNA, 20 bp, Artificial Sequence
  Description of Artificial Sequence: Synthetic oligonucleotide
gacattggcagttgtttcag

SEQ ID NO: 913 - DNA, 20 bp, Artificial Sequence
  Description of Artificial Sequence: Synthetic oligonucleotide
ggtagcatcctgtaggacat

SEQ ID NO: 914 - DNA, 20 bp, Artificial Sequence
  Description of Artificial Sequence: Synthetic oligonucleotide
gagcctttccttacgggtag

SEQ ID NO: 915 - DNA, 20 bp, Artificial Sequence
  Description of Artificial Sequence: Synthetic oligonucleotide
cttggagtcttctaggagcc

SEQ ID NO: 916 - DNA, 20 bp, Artificial Sequence
  Description of Artificial Sequence: Synthetic oligonucleotide
cagctcttttactcccttgg

SEQ ID NO: 917 - DNA, 20 bp, Artificial Sequence
  Description of Artificial Sequence: Synthetic oligonucleotide
ttgccattgtttcatcagct

SEQ ID NO: 918 - DNA, 20 bp, Artificial Sequence
  Description of Artificial Sequence: Synthetic oligonucleotide
cttacttgccattgtttcat

SEQ ID NO: 919 - DNA, 20 bp, Artificial Sequence
  Description of Artificial Sequence: Synthetic oligonucleotide
cctgacttacttgccattgt

SEQ ID NO: 920 - DNA, 20 bp, Artificial Sequence
  Description of Artificial Sequence: Synthetic oligonucleotide
aaatgcctgacttacttgcc

SEQ ID NO: 921 - DNA, 20 bp, Artificial Sequence
  Description of Artificial Sequence: Synthetic oligonucleotide
agcggaaatgcctgacttac

SEQ ID NO: 922 - DNA, 20 bp, Artificial Sequence
  Description of Artificial Sequence: Synthetic oligonucleotide
gctaaagcggaaatgcctga

SEQ ID NO: 923 - DNA, 29 bp, Artificial Sequence
  Description of Artificial Sequence: Synthetic oligonucleotide
ccactcagagctcagatcttctaacttcc

SEQ ID NO: 924 - DNA, 25 bp, Artificial Sequence
  Description of Artificial Sequence: Synthetic oligonucleotide
gggatccagtatacttacaggctcc

SEQ ID NO: 925 - DNA, 27 bp, Artificial Sequence
  Description of Artificial Sequence: Synthetic oligonucleotide
cttccactcagagctcagatcttctaa

SEQ ID NO: 926 - DNA, 30 bp, Artificial Sequence
  Description of Artificial Sequence: Synthetic oligonucleotide
acatcaaggaagatggcatttctagtttgg

SEQ ID NO: 927 - DNA, 30 bp, Artificial Sequence
  Description of Artificial Sequence: Synthetic oligonucleotide
ctccaacatcaaggaagatggcatttctag

SEQ ID NO: 928 - DNA, 24 bp, Artificial Sequence
  Description of Artificial Sequence: Synthetic oligonucleotide
ttctgtccaagcccggttgaaatc

SEQ ID NO: 929 - DNA, 21 bp, Artificial Sequence
  Description of Artificial Sequence: Synthetic oligonucleotide
cacccaccatcaccctcygtg

SEQ ID NO: 930 - DNA, 22 bp, Artificial Sequence
  Description of Artificial Sequence: Synthetic oligonucleotide
atcatctcgttgatatcctcaa

SEQ ID NO: 931 - DNA, 25 bp, Artificial Sequence
  Description of Artificial Sequence: Synthetic oligonucleotide
acatcaaggaagatggcatttctag

SEQ ID NO: 932 - DNA, 26 bp, Artificial Sequence
  Description of Artificial Sequence: Synthetic oligonucleotide
accagagtaacagtctgagtaggagc

SEQ ID NO: 933 - DNA, 20 bp, Artificial Sequence
  Description of Artificial Sequence: Synthetic oligonucleotide
tcaaggaagatggcatttct

SEQ ID NO: 934 - DNA, 23 bp, Artificial Sequence
  Description of Artificial Sequence: Synthetic oligonucleotide
cctctgtgattttataacttgat

SEQ ID NO: 935 - DNA, 25 bp, Artificial Sequence
  Description of Artificial Sequence: Synthetic oligonucleotide
atcattttttctcataccttctgct

SEQ ID NO: 936 - DNA, 23 bp, Artificial Sequence
  Description of Artificial Sequence: Synthetic oligonucleotide
ctcataccttctgcttgatgatc

SEQ ID NO: 937 - DNA, 17 bp, Artificial Sequence
  Description of Artificial Sequence: Synthetic oligonucleotide
tggcatttctagtttgg

SEQ ID NO: 938 - DNA, 23 bp, Artificial Sequence
  Description of Artificial Sequence: Synthetic oligonucleotide
ccagagcaggtacctccaacatc

SEQ ID NO: 939 - DNA, 24 bp, Artificial Sequence
  Description of Artificial Sequence: Synthetic oligonucleotide
tgttcagcttctgttagccactga

SEQ ID NO: 940 - DNA, 20 bp, Artificial Sequence
  Description of Artificial Sequence: Synthetic oligonucleotide
tttgtgtctttctgagaaac

SEQ ID NO: 941 - DNA, 19 bp, Artificial Sequence
  Description of Artificial Sequence: Synthetic oligonucleotide
cgccgccatttctcaacag

SEQ ID NO: 942 - DNA, 20 bp, Artificial Sequence
  Description of Artificial Sequence: Synthetic oligonucleotide
atctgtcaaatcgcctgcag

SEQ ID NO: 943 - DNA, 20 bp, Artificial Sequence
  Description of Artificial Sequence: Synthetic oligonucleotide
tgtttttgaggattgctgaa

SEQ ID NO: 944 - DNA, 19 bp, Artificial Sequence
  Description of Artificial Sequence: Synthetic oligonucleotide
gctgaattatttcttcccc

SEQ ID NO: 945 - DNA, 17 bp, Artificial Sequence
  Description of Artificial Sequence: Synthetic oligonucleotide
gcccaatgccatcctgg

SEQ ID NO: 946 - DNA, 26 bp, Artificial Sequence
  Description of Artificial Sequence: Synthetic oligonucleotide
ccaatgccatcctggagttcctgtaa

SEQ ID NO: 947 - DNA, 31 bp, Artificial Sequence
  Description of Artificial Sequence: Synthetic oligonucleotide
cattcaactgttgcctccggttctgaaggtg

SEQ ID NO: 948 - DNA, 25 bp, Artificial Sequence
  Description of Artificial Sequence: Synthetic oligonucleotide
ctgaaggtgttcttgtacttcatcc

SEQ ID NO: 949 - DNA, 18 bp, Artificial Sequence
  Description of Artificial Sequence: Synthetic oligonucleotide
ctgttgcctccggttctg

SEQ ID NO: 950 - DNA, 22 bp, Artificial Sequence
  Description of Artificial Sequence: Synthetic oligonucleotide
attctttcaactagaataaaag

SEQ ID NO: 951 - DNA, 30 bp, Artificial Sequence
  Description of Artificial Sequence: Synthetic oligonucleotide
gccatcctggagttcctgtaagataccaaa

SEQ ID NO: 952 - DNA, 30 bp, Artificial Sequence
  Description of Artificial Sequence: Synthetic oligonucleotide
ccaatgccatcctggagttcctgtaagata

SEQ ID NO: 953 - DNA, 30 bp, Artificial Sequence
  Description of Artificial Sequence: Synthetic oligonucleotide
gccgctgcccaatgccatcctggagttcct

SEQ ID NO: 954 - DNA, 30 bp, Artificial Sequence
  Description of Artificial Sequence: Synthetic oligonucleotide
gtttgccgctgcccaatgccatcctggagt

SEQ ID NO: 955 - DNA, 30 bp, Artificial Sequence
  Description of Artificial Sequence: Synthetic oligonucleotide
caacagtttgccgctgcccaatgccatcct

SEQ ID NO: 956 - DNA, 30 bp, Artificial Sequence
  Description of Artificial Sequence: Synthetic oligonucleotide
ctgacaacagtttgccgctgcccaatgcca

SEQ ID NO: 957 - DNA, 30 bp, Artificial Sequence
  Description of Artificial Sequence: Synthetic oligonucleotide
tgttctgacaacagtttgccgctgcccaat

SEQ ID NO: 958 - DNA, 30 bp, Artificial Sequence
  Description of Artificial Sequence: Synthetic oligonucleotide
caatgttctgacaacagtttgccgctgccc

SEQ ID NO: 959 - DNA, 30 bp, Artificial Sequence
  Description of Artificial Sequence: Synthetic oligonucleotide
cattcaatgttctgacaacagtttgccgct

SEQ ID NO: 960 - DNA, 30 bp, Artificial Sequence
  Description of Artificial Sequence: Synthetic oligonucleotide
tatttcttccccagttgcattcaatgttct

SEQ ID NO: 961 - DNA, 30 bp, Artificial Sequence
  Description of Artificial Sequence: Synthetic oligonucleotide
gctgaattatttcttccccagttgcattca

SEQ ID NO: 962 - DNA, 30 bp, Artificial Sequence
  Description of Artificial Sequence: Synthetic oligonucleotide
ggattgctgaattatttcttccccagttgc

SEQ ID NO: 963 - DNA, 30 bp, Artificial Sequence
  Description of Artificial Sequence: Synthetic oligonucleotide
tttgaggattgctgaattatttcttcccca

SEQ ID NO: 964 - DNA, 30 bp, Artificial Sequence
  Description of Artificial Sequence: Synthetic oligonucleotide
gtacttcatcccactgattctgaattcttt

SEQ ID NO: 965 - DNA, 30 bp, Artificial Sequence
  Description of Artificial Sequence: Synthetic oligonucleotide
tcttgtacttcatcccactgattctgaatt

SEQ ID NO: 966 - DNA, 30 bp, Artificial Sequence
  Description of Artificial Sequence: Synthetic oligonucleotide
tgttcttgtacttcatcccactgattctga

SEQ ID NO: 967 - DNA, 30 bp, Artificial Sequence
  Description of Artificial Sequence: Synthetic oligonucleotide
cggttctgaaggtgttcttgtacttcatcc

SEQ ID NO: 968 - DNA, 30 bp, Artificial Sequence
  Description of Artificial Sequence: Synthetic oligonucleotide
ctccggttctgaaggtgttcttgtacttca

SEQ ID NO: 969 - DNA, 30 bp, Artificial Sequence
  Description of Artificial Sequence: Synthetic oligonucleotide
tgcctccggttctgaaggtgttcttgtact

SEQ ID NO: 970 - DNA, 30 bp, Artificial Sequence
  Description of Artificial Sequence: Synthetic oligonucleotide
tgttgcctccggttctgaaggtgttcttgt

SEQ ID NO: 971 - DNA, 30 bp, Artificial Sequence
  Description of Artificial Sequence: Synthetic oligonucleotide
aactgttgcctccggttctgaaggtgttct

SEQ ID NO: 972 - DNA, 30 bp, Artificial Sequence
  Description of Artificial Sequence: Synthetic oligonucleotide
ttcaactgttgcctccggttctgaaggtgt

SEQ ID NO: 973 - protein, 4 aa, Artificial Sequence
  Description of Artificial Sequence: Synthetic peptide
GGFG

SEQ ID NO: 974 - protein, 4 aa, Artificial Sequence
  Description of Artificial Sequence: Synthetic peptide
ALAL

SEQ ID NO: 975 - protein, 4 aa, Artificial Sequence
  Description of Artificial Sequence: Synthetic peptide
GFLG

SEQ ID NO: 976 - DNA, 26 bp, Artificial Sequence
  Description of Artificial Sequence: Synthetic oligonucleotide
gggaaattacagaatcacataaaaac

SEQ ID NO: 977 - DNA, 29 bp, Artificial Sequence
  Description of Artificial Sequence: Synthetic oligonucleotide
gggaaattacaggctctgcaaagttcttt

SEQ ID NO: 978-999 - skipped (22 entries)

SEQ ID NO: 1000 - protein, 12 aa, Artificial Sequence
  Description of Artificial Sequence: Synthetic peptide
RRRRRRRRRRRR

SEQ ID NO: 1001 - protein, 27 aa, Artificial Sequence
  Description of Artificial Sequence: Synthetic peptide
GLAFLGFLGAAGSTMGAWSQPKKKRKV

SEQ ID NO: 1002 - protein, 24 aa, Artificial Sequence
  Description of Artificial Sequence: Synthetic peptide
RRIRPRPPRLPRPRPRPLPFPRPG

SEQ ID NO: 1003 - protein, 9 aa, Artificial Sequence
  Description of Artificial Sequence: Synthetic peptide
RKKRRQRRR

SEQ ID NO: 1004 - protein, 10 aa, Artificial Sequence
  Description of Artificial Sequence: Synthetic peptide
RRRRRRRRRR

SEQ ID NO: 1005 - protein, 17 aa, Artificial Sequence
  Description of Artificial Sequence: Synthetic peptide
GRPRESGKKRKRKRLKP

SEQ ID NO: 1006 - protein, 20 aa, Artificial Sequence
  Description of Artificial Sequence: Synthetic peptide
ALWKTLLKKVLKAPKKKRKV

SEQ ID NO: 1007 - protein, 10 aa, Artificial Sequence
  Description of Artificial Sequence: Synthetic peptide
RRIPNRRPRR

SEQ ID NO: 1008 - protein, 13 aa, Artificial Sequence
  Description of Artificial Sequence: Synthetic peptide
TRRQRTRRARRNR

SEQ ID NO: 1009 - protein, 20 aa, Artificial Sequence
  Description of Artificial Sequence: Synthetic peptide
HARIKPTFRRLKWKYKGKFW

SEQ ID NO: 1010 - protein, 26 aa, Artificial Sequence
  Description of Artificial Sequence: Synthetic peptide
GIGAVLKVLTTGLPALISWIKRKRQQ

SEQ ID NO: 1011 - protein, 16 aa, Artificial Sequence
  Description of Artificial Sequence: Synthetic peptide
LRRERQSRLRRERQSR

SEQ ID NO: 1012 - protein, 9 aa, Artificial Sequence
  Description of Artificial Sequence: Synthetic peptide
RRRRRRRRR

SEQ ID NO: 1013 - protein, 16 aa, Artificial Sequence
  Description of Artificial Sequence: Synthetic peptide
RQIKIWFQNRRMKWKK

SEQ ID NO: 1014 - protein, 22 aa, Artificial Sequence
  Description of Artificial Sequence: Synthetic peptide
KRARNTEAARRSRARKLQRMKQ

SEQ ID NO: 1015 - protein, 16 aa, Artificial Sequence
  Description of Artificial Sequence: Synthetic peptide
RHIKIWFQNRRMKWKK

SEQ ID NO: 1016 - protein, 8 aa, Artificial Sequence
  Description of Artificial Sequence: Synthetic peptide
RRRRRRRR

SEQ ID NO: 1017 - protein, 19 aa, Artificial Sequence
  Description of Artificial Sequence: Synthetic peptide
KMTRAQRRAAARRNRWTAR

SEQ ID NO: 1018 - protein, 18 aa, Artificial Sequence
  Description of Artificial Sequence: Synthetic peptide
RGGRLSYSRRRFSTSTGR

SEQ ID NO: 1019 - protein, 16 aa, Artificial Sequence
  Description of Artificial Sequence: Synthetic peptide
KQINNWFINQRKRHWK

SEQ ID NO: 1020 - protein, 15 aa, Artificial Sequence
  Description of Artificial Sequence: Synthetic peptide
KLWMRWYSPTTRRYG

SEQ ID NO: 1021 - protein, 9 aa, Artificial Sequence
  Description of Artificial Sequence: Synthetic peptide
RRWWRRWRR

SEQ ID NO: 1022 - protein, 16 aa, Artificial Sequence
  Description of Artificial Sequence: Synthetic peptide
SQIKIWFQNKRAKIKK

SEQ ID NO: 1023 - protein, 22 aa, Artificial Sequence
  Description of Artificial Sequence: Synthetic peptide
GAYDLRRRERQSRLRRRERQSR

SEQ ID NO: 1024 - protein, 16 aa, Artificial Sequence
  Description of Artificial Sequence: Synthetic peptide
TRRNKRNRIQEQLNRK

SEQ ID NO: 1025 - protein, 15 aa, Artificial Sequence
  Description of Artificial Sequence: Synthetic peptide
GKRKKKGKLGKKRDP

SEQ ID NO: 1026 - protein, 16 aa, Artificial Sequence
  Description of Artificial Sequence: Synthetic peptide
RQVTIWFQNRRVKEKK

SEQ ID NO: 1027 - protein, 5 aa, Artificial Sequence
  Description of Artificial Sequence: Synthetic peptide
RLRWR

SEQ ID NO: 1028 - protein, 15 aa, Artificial Sequence
  Description of Artificial Sequence: Synthetic peptide
PPRPPRPPRPPRPPR

SEQ ID NO: 1029 - protein, 9 aa, Artificial Sequence
  Description of Artificial Sequence: Synthetic peptide
CAYHRLRRC

SEQ ID NO: 1030 - protein, 14 aa, Artificial Sequence
  Description of Artificial Sequence: Synthetic peptide
SRRARRSPRHLGSG

SEQ ID NO: 1031 - protein, 12 aa, Artificial Sequence
  Description of Artificial Sequence: Synthetic peptide
PPRPPRPPRPPR

SEQ ID NO: 1032 - protein, 17 aa, Artificial Sequence
  Description of Artificial Sequence: Synthetic peptide
NAKTRRHERRRKLAIER

SEQ ID NO: 1033 - protein, 19 aa, Artificial Sequence
  Description of Artificial Sequence: Synthetic peptide
VKRGLKLRHVRPRVTRMDV

SEQ ID NO: 1034 - protein, 19 aa, Artificial Sequence
  Description of Artificial Sequence: Synthetic peptide
LYKKGPAKKGRPPLRGWFH

SEQ ID NO: 1035 - protein, 18 aa, Artificial Sequence
  Description of Artificial Sequence: Synthetic peptide
TAKTRYKARRAELIAERR

SEQ ID NO: 1036 - protein, 16 aa, Artificial Sequence
  Description of Artificial Sequence: Synthetic peptide
KGTYKKKLMRIPLKGT

SEQ ID NO: 1037 - protein, 9 aa, Artificial Sequence
  Description of Artificial Sequence: Synthetic peptide
PPRPPRPPR

SEQ ID NO: 1038 - protein, 18 aa, Artificial Sequence
  Description of Artificial Sequence: Synthetic peptide
RASKRDGSWVKKLHRILE

SEQ ID NO: 1039 - protein, 21 aa, Artificial Sequence
  Description of Artificial Sequence: Synthetic peptide
TRSSRAGLQWPVGRVHRLLRK

SEQ ID NO: 1040 - protein, 15 aa, Artificial Sequence
  Description of Artificial Sequence: Synthetic peptide
FKIYDKKVRTRVVKH

SEQ ID NO: 1041 - protein, 18 aa, Artificial Sequence
  Description of Artificial Sequence: Synthetic peptide
VRLPPPVRLPPPVRLPPP

SEQ ID NO: 1042 - protein, 13 aa, Artificial Sequence
  Description of Artificial Sequence: Synthetic peptide
GPFHFYQFLFPPV

SEQ ID NO: 1043 - protein, 12 aa, Artificial Sequence
  Description of Artificial Sequence: Synthetic peptide
PLILLRLLRGQF

SEQ ID NO: 1044 - protein, 16 aa, Artificial Sequence
  Description of Artificial Sequence: Synthetic peptide
YTAIAWVKAFIRKLRK

SEQ ID NO: 1045 - protein, 20 aa, Artificial Sequence
  Description of Artificial Sequence: Synthetic peptide
KETWWETWWTEWSQPKKRKV

SEQ ID NO: 1046 - protein, 23 aa, Artificial Sequence
  Description of Artificial Sequence: Synthetic peptide
LIRLWSHLIHIWFQNRRLKWKKK

SEQ ID NO: 1047 - protein, 20 aa, Artificial Sequence
  Description of Artificial Sequence: Synthetic peptide
VDKGSYLPRPTPPRPIYNRN

SEQ ID NO: 1048 - protein, 22 aa, Artificial Sequence
  Description of Artificial Sequence: Synthetic peptide
MDAQTRRRERRAEKQAQWKAAN

SEQ ID NO: 1049 - protein, 13 aa, Artificial Sequence
  Description of Artificial Sequence: Synthetic peptide
GSPWGLQHHPPRT

SEQ ID NO: 1050 - protein, 17 aa, Artificial Sequence
  Description of Artificial Sequence: Synthetic peptide
KLALKALKALKAALKLA

SEQ ID NO: 1051 - protein, 5 aa, Artificial Sequence
  Description of Artificial Sequence: Synthetic peptide
IPALK

SEQ ID NO: 1052 - protein, 5 aa, Artificial Sequence
  Description of Artificial Sequence: Synthetic peptide
VPALR

SEQ ID NO: 1053 - protein, 18 aa, Artificial Sequence
  Description of Artificial Sequence: Synthetic peptide
LLIILRRRIRKQAHAHSK

SEQ ID NO: 1054 - protein, 17 aa, Artificial Sequence
  Description of Artificial Sequence: Synthetic peptide
IAWVKAFIRKLRKGPLG

SEQ ID NO: 1055 - protein, 21 aa, Artificial Sequence
  Description of Artificial Sequence: Synthetic peptide
AAVLLPVLLAAPVQRKRQKLP

SEQ ID NO: 1056 - protein, 12 aa, Artificial Sequence
  Description of Artificial Sequence: Synthetic peptide
TSPLNIHNGQKL

SEQ ID NO: 1057 - protein, 5 aa, Artificial Sequence
  Description of Artificial Sequence: Synthetic peptide
VPTLK

SEQ ID NO: 1058 - protein, 5 aa, Artificial Sequence
  Description of Artificial Sequence: Synthetic peptide
VSALK

SEQ ID NO: 1059 - protein, 26 aa, Artificial Sequence
  Description of Artificial Sequence: Synthetic peptide
DPKGDPKGVTVTVTVTVTGKGDPKPD

SEQ ID NO: 1060 - protein, 21 aa, Artificial Sequence
  Description of Artificial Sequence: Synthetic peptide
AGYLLGKINLKALAALAKKIL

SEQ ID NO: 1061 - protein, 18 aa, Artificial Sequence
  Description of Artificial Sequence: Synthetic peptide
VELPPPVELPPPVELPPP

SEQ ID NO: 1062 - protein, 20 aa, Artificial Sequence
  Description of Artificial Sequence: Synthetic peptide
GLWRALWRLLRSLWRLLWRA

SEQ ID NO: 1063 - protein, 12 aa, Artificial Sequence
  Description of Artificial Sequence: Synthetic peptide
PLSSIFSRIGDP

SEQ ID NO: 1064 - skipped

SEQ ID NO: 1065 - protein, 14 aa, Artificial Sequence
  Description of Artificial Sequence: Synthetic peptide
  MOD_RES (2)..(2): 6-aminohexanoic acid
  MOD_RES (5)..(5): 6-aminohexanoic acid
  MOD_RES (8)..(8): 6-aminohexanoic acid
  MOD_RES (11)..(11): 6-aminohexanoic acid
  MOD_RES (13)..(13): 6-aminohexanoic acid
  MOD_RES (14)..(14): Beta-Alanine
RXRRXRRXRRXRXA

SEQ ID NO: 1066 - protein, 14 aa, Artificial Sequence
  Description of Artificial Sequence: Synthetic peptide
  MOD_RES (2)..(2): 6-aminohexanoic acid
  MOD_RES (5)..(5): 6-aminohexanoic acid
  MOD_RES (8)..(8): 6-aminohexanoic acid
  MOD_RES (11)..(11): 6-aminohexanoic acid
  MOD_RES (13)..(13): 6-aminohexanoic acid
  MOD_RES (14)..(14): Beta-Alanine
RXRRXRRXRRXRXA

SEQ ID NO: 1067 - protein, 22 aa, Artificial Sequence
  Description of Artificial Sequence: Synthetic peptide
  MOD_RES (2)..(2): 6-aminohexanoic acid
  MOD_RES (5)..(5): Beta-Alanine
  MOD_RES (8)..(8): 6-aminohexanoic acid
  MOD_RES (16)..(16): 6-aminohexanoic acid
  MOD_RES (18)..(18): Beta-Alanine
  MOD_RES (20)..(20): 6-aminohexanoic acid
  MOD_RES (22)..(22): Beta-Alanine
RXRRARRXRYQFLIRXRARXRA

SEQ ID NO: 1068 - DNA, 20 bp, Artificial Sequence
  Description of Artificial Sequence: Synthetic primer
tctggatgcagactttgtgg

SEQ ID NO: 1069 - DNA, 27 bp, Artificial Sequence
  Description of Artificial Sequence: Synthetic primer
gtttttatgtgattttgtctgtagctc

SEQ ID NO: 1070 - DNA, 23 bp, Artificial Sequence
  Description of Artificial Sequence: Synthetic probe
taaccacatctttgatggtgtga

SEQ ID NO: 1071 - protein, 16 aa, Artificial Sequence
  Description of Artificial Sequence: Synthetic peptide
  MOD_RES (2)..(2): Citrulline
  MOD_RES (4)..(4): 6-aminohexanoic acid
  MOD_RES (7)..(7): 6-aminohexanoic acid
  MOD_RES (10)..(10): 6-aminohexanoic acid
  MOD_RES (13)..(13): 6-aminohexanoic acid
  MOD_RES (15)..(15): 6-aminohexanoic acid
  MOD_RES (16)..(16): Beta-Alanine
VXRXRRXRRXRRXRXA

SEQ ID NO: 1072 - protein, 6 aa, Artificial Sequence
  Description of Artificial Sequence: Synthetic peptide
RRRRRR